(19) United States
(12) Patent Application Publication
(10) Pub. No.: US 2025/0026769 A1
(43) Pub. Date: Jan. 23, 2025

(54) PAPD5 INHIBITORS AND METHODS OF USE THEREOF

(71) Applicant: The Children's Medical Center Corporation, Boston, MA (US)

(72) Inventors: Suneet Agarwal, Lexington, MA (US); John J. Piwinski, Lebanon, NJ (US); Neha Nagpal, Boston, MA (US)

(21) Appl. No.: 18/701,011

(22) PCT Filed: Oct. 28, 2022
(86) PCT No.: PCT/US22/48187

§ 371 Date: Apr. 12, 2024

Related U.S. Application Data
(60) Provisional application No. 63/273,871, filed on Oct. 29, 2021.

(51) Int. Cl. C07F 7/08; A61K 31/4709; A61K 31/5377; A61K 31/541; A61K 31/695; C07D 215/44; C07D 401/04; C07D 405/04; C07D 409/04; C07D 413/04; C07D 417/04

(52) U.S. Cl. CPC C07F 7/0836; A61K 31/4709; A61K 31/5377; A61K 31/541; A61K 31/695; C07D 215/44; C07D 401/04; C07D 405/04; C07D 409/04; C07D 413/04; C07D 417/04

(57) ABSTRACT

The present application provides compounds that are PAPD5 inhibitors and are useful in treating a variety of conditions such as cancer, telomere diseases, viral infections, and aging-related and other degenerative disorders.

### CLAIM OF PRIORITY

This application claims priority to U.S. Provisional Patent Application Ser. No. 63/273,871, filed on Oct. 29, 2021, the entire contents of which are hereby incorporated by reference.

### FEDERALLY SPONSORED RESEARCH AND DEVELOPMENT

This invention was made with government support under Grant nos. DK107716, HL119145, and HL154133, awarded by The National Institutes of Health; and under Grant no. W81XWH-19-1-0572, awarded by the U.S. Department of the Army. The government has certain rights in the invention.

### TECHNICAL FIELD

The present disclosure relates to compounds that inhibit PAP Associated Domain Containing 5 (PAPD5), and to methods of using these compounds to treat conditions such as telomere diseases, viral diseases, and aging-related and other degenerative disorders.

### BACKGROUND

A telomere is a region of repetitive nucleotide sequences at each end of a chromosome, which protects the end of the chromosome from deterioration or from fusion with neighboring chromosomes. The length of a telomere is a key determinant of cellular self-renewal capacity. The telomerase ribonucleoprotein maintains telomere length in tissue stem cells, and its function is critical for human health and longevity.

Short telomeres, due to genetic or acquired insults, cause a loss of cellular self-renewal and result in life-threatening diseases, for which there are few if any effective medical therapies. In these diseases involving short telomeres, e.g., aplastic anemia, pulmonary fibrosis, hepatic cirrhosis, bone marrow failure, etc., there is an unmet clinical need for new therapies.

### SUMMARY

Poly(A) ribonuclease (PARN) mutations can result in the accumulation of 3′ oligo-adenylated forms of nascent Telomerase RNA Component (TERC) RNA transcripts, which are targeted for destruction, thus causing telomerase deficiency and telomere diseases. Disruption of the non-canonical poly(A) polymerase PAP Associated Domain Containing 5 (PAPD5; also known as Topoisomerase-related function protein 4-2 (TRF4-2)) may restore TERC levels, telomerase activity, and telomere elongation in PARN-mutant patient cells. This disclosure relates, at least in part, to PAPD5 inhibitors and methods of using such inhibitors.

In some embodiments, the present disclosure provides a compound of Formula (I):

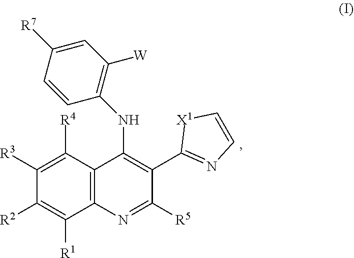

or a pharmaceutically acceptable salt thereof.

In some embodiments, the present disclosure provides a compound of Formula (II):

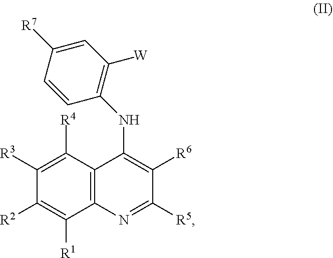

or a pharmaceutically acceptable salt thereof.

In some embodiments, the present disclosure provides a compound of Formula (III):

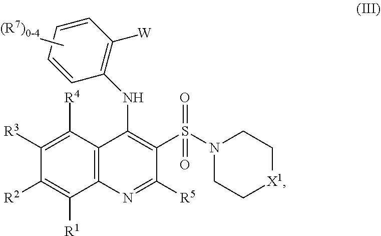

or a pharmaceutically acceptable salt thereof.

In some embodiments, the present disclosure provides a compound of Formula (IV):

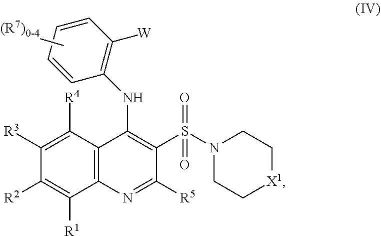

or a pharmaceutically acceptable salt thereof.

In some embodiments, the present disclosure provides a compound of Formula (V):

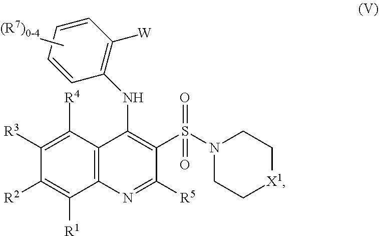

or a pharmaceutically acceptable salt thereof.

In some embodiments, the present disclosure provides a compound of Formula (VI):

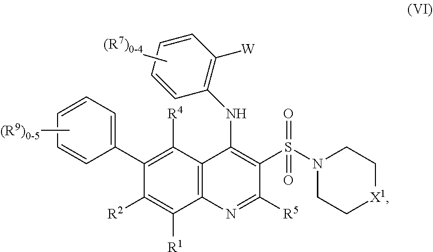

or a pharmaceutically acceptable salt thereof.

In some embodiments, the present disclosure provides a compound of Formula (VII):

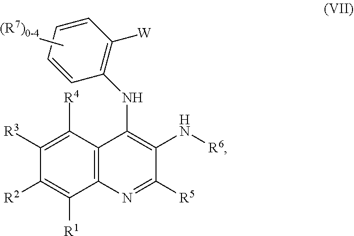

or a pharmaceutically acceptable salt thereof.

In some embodiments, the present disclosure provides a compound of Formula (VIII):

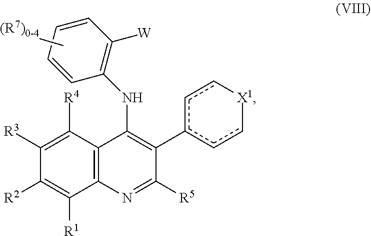

or a pharmaceutically acceptable salt thereof.

In some embodiments, the present disclosure provides a compound of Formula (IX):

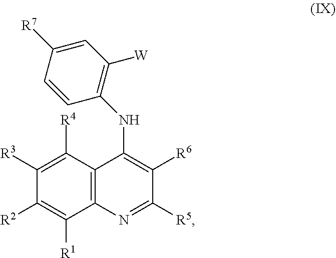

or a pharmaceutically acceptable salt thereof.

In some embodiments, the present disclosure provides a compound of Formula (X):

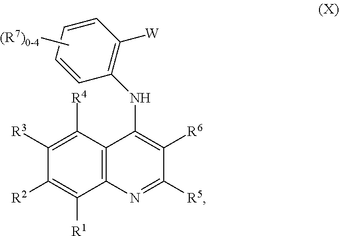

or a pharmaceutically acceptable salt thereof.

In some embodiments, the present disclosure provides a compound of Formula (I):

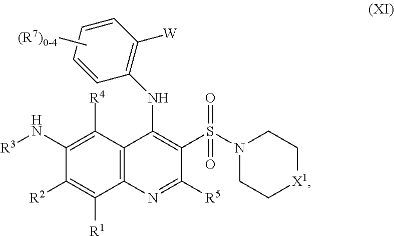

or a pharmaceutically acceptable salt thereof.

In some embodiments, the present disclosure provides a compound of Formula (I):

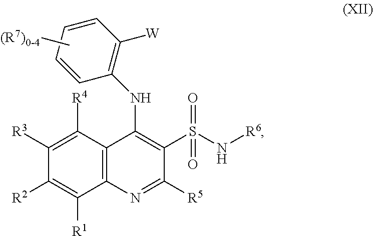

or a pharmaceutically acceptable salt thereof.

In some embodiments, the present disclosure provides a compound of Formula (XIII):

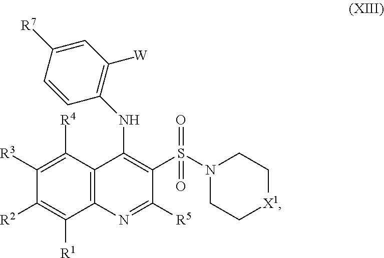

or a pharmaceutically acceptable salt thereof.

In some embodiments, the present disclosure provides a compound of Formula (XIV):

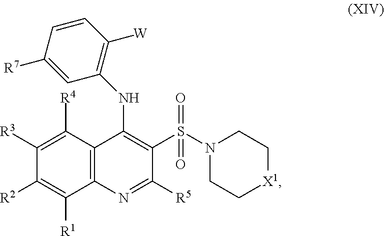

or a pharmaceutically acceptable salt thereof.

In some embodiments, the present disclosure provides a compound of Formula (XV):

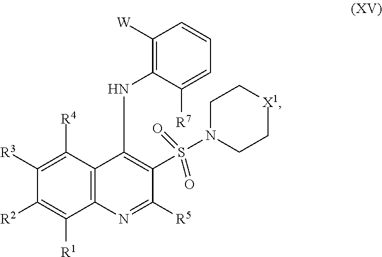

or a pharmaceutically acceptable salt thereof.

In some embodiments, the present disclosure provides a compound of Formula (XVI):

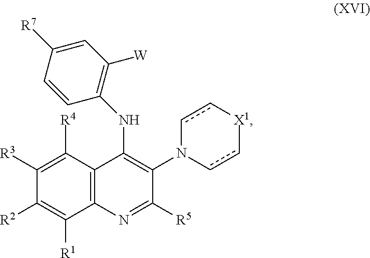

or a pharmaceutically acceptable salt thereof.

In some embodiments, the present disclosure provides a compound of Formula (XVII):

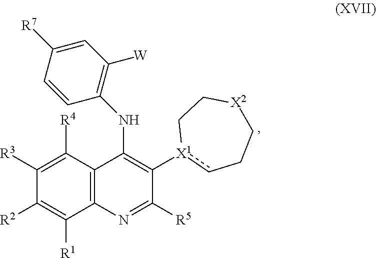

or a pharmaceutically acceptable salt thereof.

In some embodiments, the present disclosure provides a compound of Formula (XVIII):

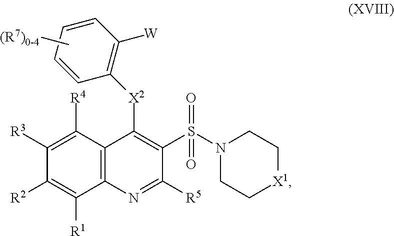

or a pharmaceutically acceptable salt thereof.

In yet another general aspect, the present disclosure provides a pharmaceutical composition comprising a compound of any one of the Formulae described herein, or a pharmaceutically acceptable salt thereof, and a pharmaceutically acceptable carrier.

In yet another general aspect, the present disclosure provides a method selected from:
(a) treating a disorder associated with telomere or telomerase dysfunction in a subject;
(b) treating a disorder associated with aging in a subject;
(c) treating a pre-leukemic or pre-cancerous condition in subject;
(d) treating or preventing HBV infection in a subject;
(e) treating or preventing a neurodevelopmental disorder in a subject;
(f) treating an acquired or genetic disease or condition associated with alterations in RNA in a subject;
(g) decreasing PAPD5 activity in a subject;
(h) inhibiting of HBsAg production or secretion in a subject;
(i) inhibiting HBV DNA production in a subject
(j) decreasing PAPD5 activity in a cell;
(k) inhibiting of HBsAg production or secretion in a cell;
(l) inhibiting HBV DNA production in a cell;
(m) modulating non-coding RNAs in a cell;
(n) modulating ex vivo expansion of a stem cell,
(o) treating or preventing HAV infection in a subject; and
(p) treating or preventing CMV infection in a subject;
the method comprising contacting the cell with an effective amount of, or administering to a subject in need thereof a therapeutically effective amount of, a compound of any one of the Formulae described herein, or a pharmaceutically acceptable salt thereof, or a pharmaceutical composition comprising same.

In yet another general aspect, the present disclosure provides a method of expanding a cell, the method comprising culturing the cell in the presence of an effective amount of a compound of any one of the Formulae described herein, or a pharmaceutically acceptable salt thereof.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 is a schematic diagram showing an exemplary model for TERC 3′ end maturation by PARN.

FIG. 2 is a schematic diagram showing an exemplary model of reciprocal regulation of TERC maturation by PARN and PAPD5.

FIG. 3 shows results of TERC 3′ end processing—Rapid Amplification of cDNA Ends (RACE) for exemplified compounds 295A, 302A, 301A, and 300A.

FIG. 4 shows results of TERC 3′ end processing—Rapid Amplification of cDNA Ends (RACE) for exemplified compounds 266A, 267A, 269A, and 270A.

FIG. 5 shows TERC 3′ end processing—Rapid Amplification of cDNA Ends (RACE) for exemplified compounds 129A and 130A.

FIG. 6 shows results of TERC 3′RLM RACE experiments (patient iPSCs) for exemplified compounds 266A, 295A, and 296A, compared to DMSO and/or compound RG7834.

FIG. 7 shows results of RNA oligo-adenylation assay for compounds 266A and 80A. The exemplified compounds show improved potency compared to Cmpd. 1 and RG7834. Chemical structure of RG7834 is also shown.

FIG. 8 shows TERC 3′ end processing—Rapid Amplification of cDNA Ends (RACE)—and maturation by 266A in the low nM range in DC patient iPSCs.

FIG. 9 shows telomere elongation in patient iPSCs by 266A at 10 nM.

FIG. 10 shows telomere elongation in patient iPSCs by 295A and 296A at 1 nM.

FIG. 11 shows TERC 3′ end processing—Rapid Amplification of cDNA Ends (RACE) for exemplified compounds 109A, 129A, 130A, 185A, 204A-INT, 211A, 233A, 204A, 205A-INT, 209A, and 226A.

FIG. 12 shows TERC 3′ end processing—Rapid Amplification of cDNA Ends (RACE) for exemplified compounds 266A, 267A, 269A, 270A, 295A, 297A, 299A, 296A, 307A, 303A, 302A, 301A, 200A, 298A, 308A, 306A, 305A, 304A, 341A.

FIG. 13 contains results of RNA oligo-adenylation assay (rPAPD5) for exemplified compounds 130A and 131A.

FIG. 14 contains results of RNA oligo-adenylation assay (rPAPD5) for exemplified compounds 129A, 132A, and 133A.

FIG. 15 contains results of RNA oligo-adenylation assay (rPAPD5) for exemplified compounds 184A, 205A-INT, and 209A.

FIG. 16 contains results of RNA oligo-adenylation assay (rPAPD5) for exemplified compounds 212A, 216A, 221 A, 226A, 231 A.

FIG. 17 contains results of RNA oligo-adenylation assay (rPAPD5) for exemplified compounds 185A, 188A, 191A, and 204A-INT.

FIG. 18 contains results of RNA oligo-adenylation assay (rPAPD5) for exemplified compounds 211A and 233A.

FIG. 19 contains results of RNA oligo-adenylation assay (rPAPD5) for exemplified compounds 205A and 204A.

FIG. 20 contains results of RNA oligo-adenylation assay (rPAPD5) for exemplified compounds 266A, 269A, 205A-INT, and 267A.

FIG. 21 contains results of RNA oligo-adenylation assay (rPAPD5) for exemplified compound 270A.

FIG. 22 contains results of RNA oligo-adenylation assay (rPAPD5) for exemplified compounds 299A, 296A, 298A, 304A, and 306A.

FIG. 23 contains results of RNA oligo-adenylation assay (rPAPD5) for exemplified compounds 208A, 300A, 301A, 302A, 303A, 305A, 308A.

FIG. 24 contains results of RNA oligo-adenylation assay (rPAPD5) for exemplified compound 307A.

FIG. 25 contains results of RNA oligo-adenylation assay (rPAPD5) for exemplified compounds 296A, 297A, 341A, 342A, and 344A.

FIG. 26 contains results of RNA oligo-adenylation assay (rPAPD5) for exemplified compound 295A.

FIG. 27 contains results of RNA oligo-adenylation assay (rPAPD5) for exemplified compounds 121A, 123A, and 123A-CBZ.

FIG. 28 contains results of RNA oligo-adenylation assay (rPAPD5) for exemplified compounds 134A, 138A, 142A, and 129A.

FIG. 29 contains results of RNA oligo-adenylation assay (rPAPD5) for exemplified compounds 87A-Cl, 135A, 136A, 137A, and 144A.

FIG. 30 contains results of RNA oligo-adenylation assay (rPAPD5) for exemplified compounds 145A and 146A-Cl.

FIG. 31 contains results of RNA oligo-adenylation assay (rPAPD5) for exemplified compounds 139A and 140A.

FIG. 32 contains results of RNA oligo-adenylation assay (rPAPD5) for exemplified compounds 127A and 135A-BP.

FIG. 33 contains results of RNA oligo-adenylation assay (rPAPD5) for exemplified compounds 220A and 232A.

FIG. 34 contains results of RNA oligo-adenylation assay (rPAPD5) for exemplified compound 275A, 276A, 277A, 278A, and 279A.

FIG. 35 shows TERC 3′ end processing—Rapid Amplification of cDNA Ends (RACE) for exemplified compounds 296A, 297A, 344A, 353A, 354A, 349A, 391A, 392A, 393A, 404A, 361A, 367A, 371A, 339A, 340A, 343A, 394A, and 430A tested at 1 nM in PARN-mutant iPSCs on day 4.

FIG. 36 shows terminal restriction fragment (TRF) telomere length measurement (southern blot) for exemplified compounds 296A, 297A, 344A, 353A, 354A, 349A, 391A, 392A, 393A, 404A, 361A, 367A, 371A, 339A, 340A, and 343A tested at 1 nM in PARN-mutant iPSCs on day 4.

FIG. 37 shows TERC 3′ end processing—Rapid Amplification of cDNA Ends (RACE) for exemplified compounds 296A, 349A, 399A, 411A, 416A, 417A, 418A, 420A, 421A, 422A, 423A, 428A, 396A, 413A, 414A, and 419A tested at 1 nM in PARN-mutant iPSCs on day 4.

FIG. 38 shows terminal restriction fragment (TRF) telomere length measurement (southern blot) for exemplified compounds 296A, 349A, 399A, 411A, 416A, 417A, 418A, 420A, 421A, 422A, 423A, 428A, 396A, 413A, 414A, and 419A tested at 1 nM in PARN-mutant iPSCs on day 4.

FIG. 39A contains a synthetic scheme showing synthesis of compound 296A.

FIG. 39B contains a synthetic scheme showing synthesis of compound 339A

FIG. 39C contains a synthetic scheme showing synthesis of compound 340A.

FIG. 39D contains a synthetic scheme showing synthesis of compound 343A.

FIG. 39E contains a synthetic scheme showing synthesis of compound 349A.

FIG. 39F contains a synthetic scheme showing synthesis of compound 357A.

FIG. 39G contains a synthetic scheme showing synthesis of compound 362A.

FIG. 39H contains a synthetic scheme showing synthesis of compound 371A.

FIG. 39I contains a synthetic scheme showing synthesis of compound 373A.

FIG. 39J contains a synthetic scheme showing synthesis of compound 394A.

FIG. 39K contains a synthetic scheme showing synthesis of compound 396A.

FIG. 39L contains a synthetic scheme showing synthesis of compound 400A.

FIG. 39M contains a synthetic scheme showing synthesis of compound 404A.

FIG. 39N contains a synthetic scheme showing synthesis of compound 404A.

FIG. 39O contains a synthetic scheme showing synthesis of compound 411 A.

FIG. 39P contains a synthetic scheme showing synthesis of compound 413A.

FIG. 39Q contains a synthetic scheme showing synthesis of compound 415A.

FIG. 39R contains a synthetic scheme showing synthesis of compound 416A.

FIG. 39S contains a synthetic scheme showing synthesis of compound 417A.

FIG. 39T contains a synthetic scheme showing synthesis of compound 418A.

FIG. 39U contains a synthetic scheme showing synthesis of compound 419A.

FIG. 39V contains a synthetic scheme showing synthesis of compound 420A.

FIG. 39W contains a synthetic scheme showing synthesis of compound 421A.

FIG. 39X contains a synthetic scheme showing synthesis of compound 422A.

FIG. 39Y contains a synthetic scheme showing synthesis of compound 430A.

FIG. 40A shows TERC 3′ end processing—Rapid Amplification of cDNA Ends (RACE) for exemplified compounds 296A, 339A, 340A, 371A, 392A, 417A, 420A, 421A, 428A, and 396A tested at 1 μM in CRISPR/Cas9-engineered primary human hematopoietic stem and progenitor cells on day 5.

FIG. 40B shows TERC 3′ end processing—Rapid Amplification of cDNA Ends (RACE) for exemplified compounds 296A, 392A, 396A, 339A, 340A, 371A, 393A, and 404A tested at 100 nM in CRISPR/Cas9-engineered primary human hematopoietic stem and progenitor cells on day 5.

FIG. 41A shows maturation of TERC 3′ ends in human CD19+ cells recovered from mice xenotransplanted with HSPCs, for exemplified compound 296A administered at 32 mg/kg/dose twice daily for 11 doses, with simultaneous administration of 296A at 250 μM in drinking water. RACE amplicons were subjected to next-generation sequencing and oligo-adenylation was analyzed using a bioinformatics pipeline, showing that aberrant TERC oligo-adenylation in xenotransplanted PARN-deficient human blood cells was significantly reversed in vivo by exemplified compound 296A oral administration. Analysis of engraftment shows no change in engraftment of CD45+ human blood cells, CD19+ human blood cells, CD34+ human blood cells after exemplified compound 296A treatment.

FIG. 41B shows maturation of TERC 3′ ends in human CD19+ cells recovered from mice xenotransplanted with HSPCs, for exemplified compound 344 administered at 32 mg/kg/dose every other day for 4 days. RACE amplicons were subjected to next-generation sequencing and oligo-adenylation was analyzed using bioinformatics pipelines, showing aberrant TERC oligo-adenylation in xenotransplanted PARN-deficient human blood cells was significantly reversed in vivo by exemplified compound 344A oral administration. Analysis of engraftment shows no change in engraftment of CD45+ human blood cells, CD19+ human blood cells, CD34+ human blood cells after exemplified compound 344A treatment.

FIG. 41C shows maturation of TERC 3′ ends in human CD19+ cells recovered from mice xenotransplanted with HSPCs, for exemplified compound 339A administered at 1 mM in drinking water for 7 days. Analysis of engraftment shows no change in engraftment of CD45+ human blood cells, CD19+ human blood cells, CD34+ human blood cells after exemplified compound 339A treatment.

FIG. 41D shows maturation of TERC 3′ ends in human CD19+ cells recovered from mice xenotransplanted with HSPCs, for exemplified compounds 297A or 392A administered at 32 mg/kg/dose twice daily for 11 doses. Analysis of engraftment shows no change in engraftment of CD45+ human blood cells, CD19+ human blood cells, CD34+ human blood cells after exemplified compound 297A or 392A treatment.

FIG. 42 contains results of RNA oligo-adenylation assay (rPAPD5) for exemplified compounds 339A, 343A, and 345A.

FIG. 43 contains results of RNA oligo-adenylation assay (rPAPD5) for exemplified compounds 346A, 340A, and 349A.

FIG. 44 contains results of RNA oligo-adenylation assay (rPAPD5) for exemplified compounds 391A, 367A, 362A, 361A, 368A, and 354A.

FIG. 45 contains results of RNA oligo-adenylation assay (rPAPD5) for exemplified compounds 372A, 353A, 395A, and 373A.

FIG. 46 contains results of RNA oligo-adenylation assay (rPAPD5) for exemplified compounds 401A, 355A, 376A, and 399A.

FIG. 47 contains results of RNA oligo-adenylation assay (rPAPD5) for exemplified compounds 357A, 359A, 371A, 392A, 402A, and 403A.

FIG. 48 contains results of RNA oligo-adenylation assay (rPAPD5) for exemplified compounds 393A, 404A, 417A, 422A, 425A, 427A, and 429A.

FIG. 49 contains results of RNA oligo-adenylation assay (rPAPD5) for exemplified compounds 420A, 421A, 423A, and 426A.

FIG. 50 contains results of RNA oligo-adenylation assay (rPAPD5) for exemplified compounds 349A, 417A, 418A, 420A, 422A, 423A, and 428A.

FIG. 51 contains results of RNA oligo-adenylation assay (rPAPD5) for exemplified compounds 396A, 413A, 414A and 419A.

FIG. 52 contains results of RNA oligo-adenylation assay (rPAPD5) for exemplified compounds 400A, 415A, 411A, and 416A.

FIG. 53 contains results of RNA oligo-adenylation assay (rPAPD5) for exemplified compounds 394A and 430A.

### DETAILED DESCRIPTION

A telomere is a region of repetitive nucleotide sequences at each end of a chromosome. For vertebrates, the sequence of nucleotides in telomeres is TTAGGG. In humans, this sequence of TTAGGG is repeated approximately hundreds to thousands of times. Telomerase is a ribonucleoprotein that adds the telomere repeat sequence to the 3′ end of telomeres. Cells with impaired telomerase function often have limited capacity for self-renewal, i.e., an abnormal state or condition characterized by an inability of cells (e.g., stem cells) to divide sufficiently. This deficiency in cells can, for example, lead to various diseases and disorders.

Telomerase RNA component (TERC) serves at least two functions: (1) it encodes the template sequence used by telomerase reverse transcriptase (TERT) for the addition of hexanucleotide repeats to telomeres, and (2) it is the scaffold that nucleates multiple proteins that target telomerase to the Cajal body, where telomeres are extended.

The disclosure provides compounds and methods to modulate TERC levels, e.g., by using compounds that target TERC, or compounds that modulate the level or activity of PAP Associated Domain Containing 5 (PAPD5) and/or Poly(A) specific ribonuclease (PARN), both of which are involved in the 3′-end maturation of TERC. Various implementations of these compounds and methods are described herein.

### Therapeutic Compounds

In some embodiments, the present disclosure provides a compound of Formula (T):

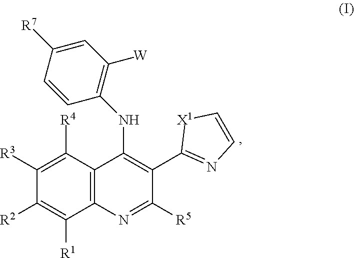

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, C₁₋₃ alkyl, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere.

In some embodiments, the carboxylic acid bioisostere is selected from a moiety of any one of the following formulae:

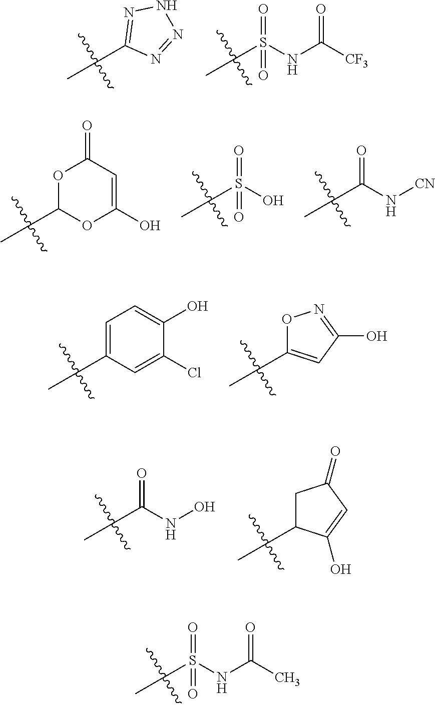

In some embodiments, the compound of Formula (I) has formula:

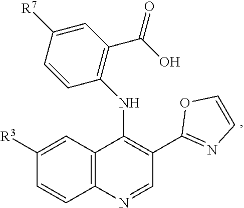

or a pharmaceutically acceptable salt thereof.

In some embodiments, the compound of Formula (I) has formula:

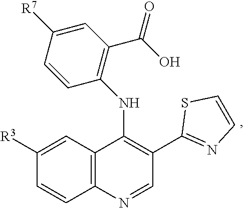

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R³ is F and R⁷ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (I) is selected from any one of the following compounds:

TABLE I

| | |
|---|---|
| BCH-295A | 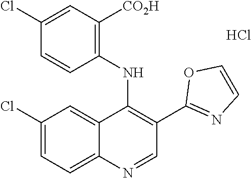 [SMILES: O=C(O)c1cc(Cl)ccc1Nc1c(-c2ncco2)cnc2ccc(Cl)cc12 ; Cl] |
| BCH-300A | 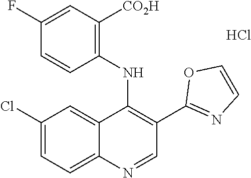 [SMILES: O=C(O)c1cc(F)ccc1Nc1c(-c2ncco2)cnc2ccc(Cl)cc12 ; Cl] |
| BCH-301A | 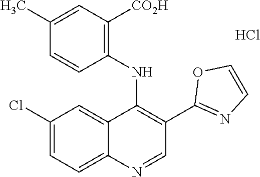 [SMILES: Cc1ccc(Nc2c(-c3ncco3)cnc3ccc(Cl)cc23)c(C(=O)O)c1 ; Cl] |
| BCH-302A | 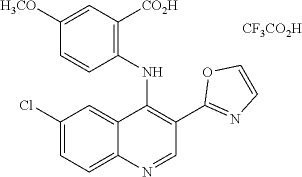 [SMILES: COc1ccc(Nc2c(-c3ncco3)cnc3ccc(Cl)cc23)c(C(=O)O)c1 ; "CF3CO2H"] |
| | 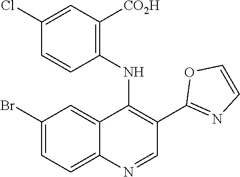 [SMILES: O=C(O)c1cc(Cl)ccc1Nc1c(-c2ncco2)cnc2ccc(Br)cc12] |
| | 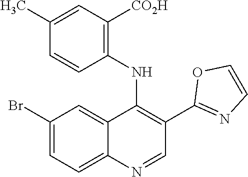 [SMILES: Cc1ccc(Nc2c(-c3ncco3)cnc3ccc(Br)cc23)c(C(=O)O)c1] |
| | 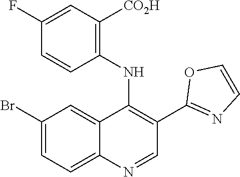 [SMILES: O=C(O)c1cc(F)ccc1Nc1c(-c2ncco2)cnc2ccc(Br)cc12] |
| | 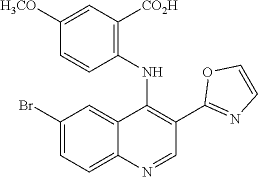 [SMILES: COc1ccc(Nc2c(-c3ncco3)cnc3ccc(Br)cc23)c(C(=O)O)c1] |
| | 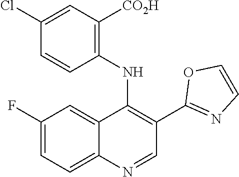 [SMILES: O=C(O)c1cc(Cl)ccc1Nc1c(-c2ncco2)cnc2ccc(F)cc12] |
| | 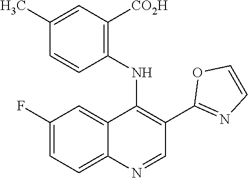 [SMILES: Cc1ccc(Nc2c(-c3ncco3)cnc3ccc(F)cc23)c(C(=O)O)c1] |
| | 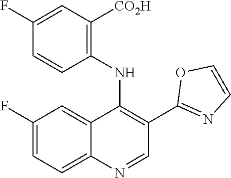 [SMILES: O=C(O)c1cc(F)ccc1Nc1c(-c2ncco2)cnc2ccc(F)cc12] |
| | 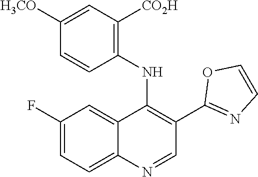 [SMILES: COc1ccc(Nc2c(-c3ncco3)cnc3ccc(F)cc23)c(C(=O)O)c1] |
| | 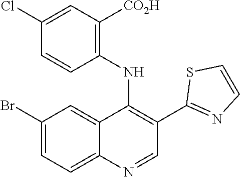 [SMILES: O=C(O)c1cc(Cl)ccc1Nc1c(-c2nccs2)cnc2ccc(Br)cc12] |
| | 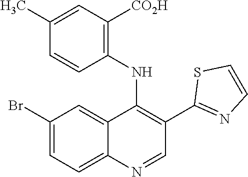 [SMILES: Cc1ccc(Nc2c(-c3nccs3)cnc3ccc(Br)cc23)c(C(=O)O)c1] |
| | 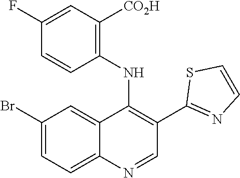 [SMILES: O=C(O)c1cc(F)ccc1Nc1c(-c2nccs2)cnc2ccc(Br)cc12] |
| | 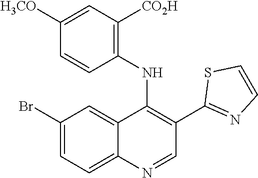 [SMILES: COc1ccc(Nc2c(-c3nccs3)cnc3ccc(Br)cc23)c(C(=O)O)c1] |
| | 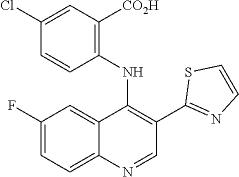 [SMILES: O=C(O)c1cc(Cl)ccc1Nc1c(-c2nccs2)cnc2ccc(F)cc12] |
| | 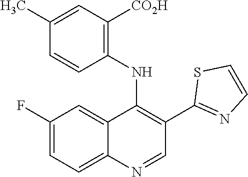 [SMILES: Cc1ccc(Nc2c(-c3nccs3)cnc3ccc(F)cc23)c(C(=O)O)c1] |
| | 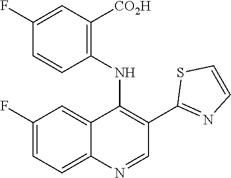 [SMILES: O=C(O)c1cc(F)ccc1Nc1c(-c2nccs2)cnc2ccc(F)cc12] |
| | 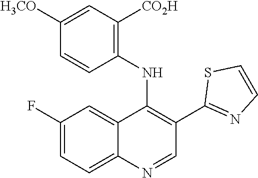 [SMILES: COc1ccc(Nc2c(-c3nccs3)cnc3ccc(F)cc23)c(C(=O)O)c1] |
| | 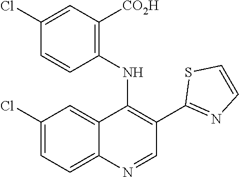 [SMILES: O=C(O)c1cc(Cl)ccc1Nc1c(-c2nccs2)cnc2ccc(Cl)cc12] |
| | 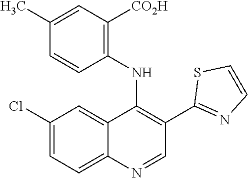 [SMILES: Cc1ccc(Nc2c(-c3nccs3)cnc3ccc(Cl)cc23)c(C(=O)O)c1] |
| | 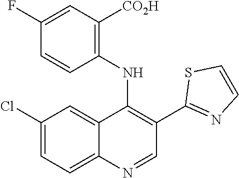 [SMILES: O=C(O)c1cc(F)ccc1Nc1c(-c2nccs2)cnc2ccc(Cl)cc12] |
| | 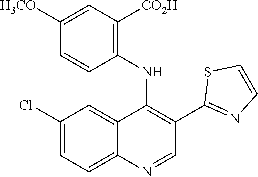 [SMILES: COc1ccc(Nc2c(-c3nccs3)cnc3ccc(Cl)cc23)c(C(=O)O)c1] |

or a pharmaceutically acceptable salt thereof.

In some embodiments, the present disclosure provides a compound of Formula (II):

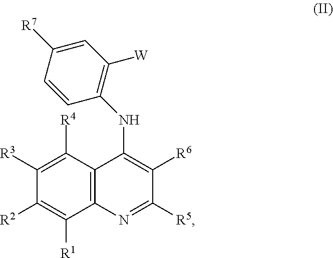

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo;
R⁶ is a 5-membered heteroaryl selected from the group consisting of:

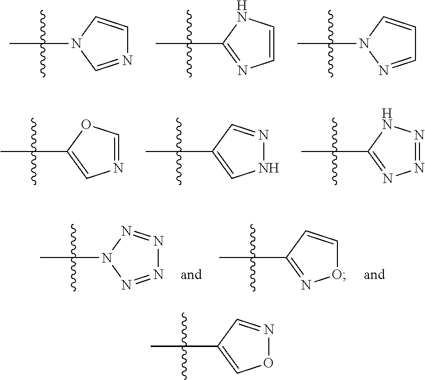

R⁷ is selected from halo, C₁₋₃ alkyl, and C₁₋₃ alkoxy.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments the compound of Formula (II) has formula:

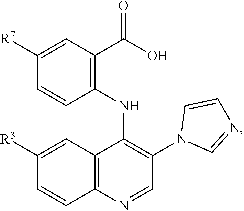

or a pharmaceutically acceptable salt thereof.

In some embodiments the compound of Formula (II) has formula:

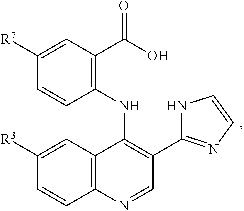

or a pharmaceutically acceptable salt thereof.

In some embodiments the compound of Formula (II) has formula:

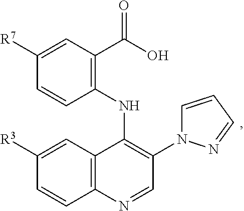

or a pharmaceutically acceptable salt thereof.

In some embodiments the compound of Formula (II) has formula:

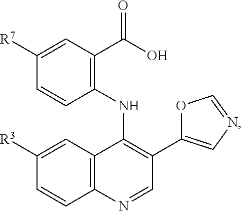

or a pharmaceutically acceptable salt thereof.

In some embodiments the compound of Formula (II) has formula:

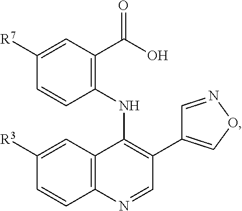

or a pharmaceutically acceptable salt thereof.

In some embodiments the compound of Formula (II) has formula:

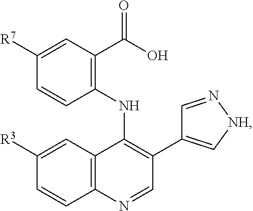

or a pharmaceutically acceptable salt thereof.

In some embodiments the compound of Formula (II) has formula:

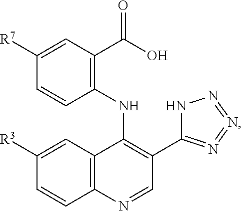

or a pharmaceutically acceptable salt thereof.

In some embodiments the compound of Formula (II) has formula:

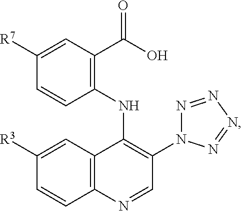

or a pharmaceutically acceptable salt thereof.

In some embodiments the compound of Formula (II) has formula:

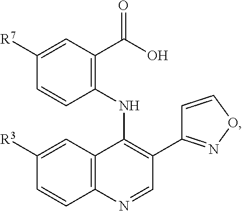

or a pharmaceutically acceptable salt thereof.

In some embodiments, R³ is selected from Cl, Br, and F. In some embodiments, R³ is Cl. In some embodiments, R³ is Br. In some embodiments, R³ is F. In some embodiments, R⁷ is halo.

In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R³ is Cl and R⁷ is Cl.

In some embodiments, the compound of Formula (II) is selected from any one of the following compounds:

TABLE II

| | |
|---|---|
| 348A | 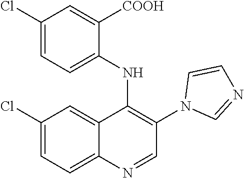 [SMILES: O=C(O)c1cc(Cl)ccc1Nc1c(-n2ccnc2)cnc2ccc(Cl)cc12] |
| BCH-134A | 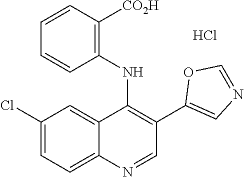 [SMILES: O=C(O)c1ccccc1Nc1c(-c2cnco2)cnc2ccc(Cl)cc12 ; Cl] |
| | 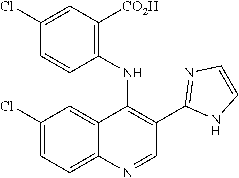 [SMILES: O=C(O)c1cc(Cl)ccc1Nc1c(-c2ncc[nH]2)cnc2ccc(Cl)cc12] |
| | 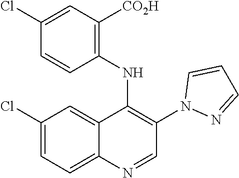 [SMILES: O=C(O)c1cc(Cl)ccc1Nc1c(-n2cccn2)cnc2ccc(Cl)cc12] |
| BCH-353A | 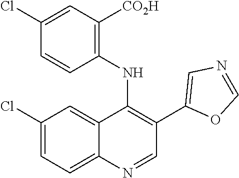 [SMILES: O=C(O)c1cc(Cl)ccc1Nc1c(-c2cnco2)cnc2ccc(Cl)cc12] |
| BCH-354A | 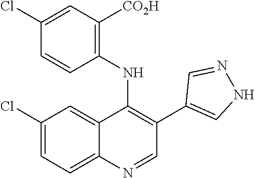 [SMILES: O=C(O)c1cc(Cl)ccc1Nc1c(-c2cn[nH]c2)cnc2ccc(Cl)cc12] |
| BCH-359A | 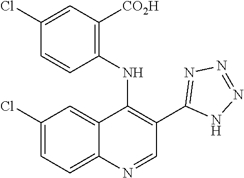 [SMILES: O=C(O)c1cc(Cl)ccc1Nc1c(-c2nnn[nH]2)cnc2ccc(Cl)cc12] |
| | 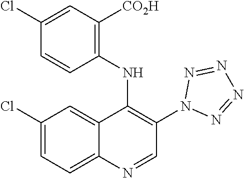 [SMILES: O=C(O)c1cc(Cl)ccc1Nc1c(-n2nnnn2)cnc2ccc(Cl)cc12] |

or a pharmaceutically acceptable salt thereof.

In some embodiments, the present disclosure provides a compound of Formula (III):

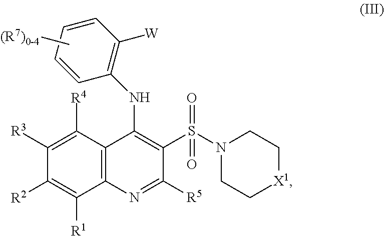

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
R³ is a 5-membered heteroaryl, optionally substituted with 1, 2, or 3 substituents independently selected from C₁₋₆ alkyl, C₁₋₄ haloalkyl, C₁₋₆ alkylcarbonyl, CN, halo, C₁₋₆ alkoxy, C₁₋₆ alkoxy-C₁₋₆ alkyl, NO₂, C₁₋₆ haloalkoxy, cyano-C₁₋₃ alkylene, C₃₋₁₀ cycloalkyl, 4-6 membered heterocycloalkyl, amino, C₁₋₆ alkylamino, di(C₁₋₆ alkyl)amino, carboxy, and C₁₋₆ alkoxycarbonyl;
W is selected from C(O)OR⁸ and a carboxylic acid bioisostere;
R⁸ is selected from H and C₁₋₆ alkyl; and
each R⁷ is selected from halo, C₁₋₃ alkyl, C₁₋₃ haloalkyl, C₁₋₃ haloalkoxy, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (III) has formula:

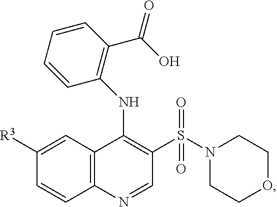

or a pharmaceutically acceptable salt thereof.

In some embodiments, R³ is a 5-membered heteroaryl, optionally substituted with 1, 2, or 3 substituents independently selected from C₁₋₆ alkyl, C₁₋₄ haloalkyl, C₁₋₆ alkylcarbonyl, CN, halo, C₁₋₆ alkoxy, 4-6 membered heterocycloalkyl and C₁₋₆ alkoxy-C₁₋₆ alkyl.

In some embodiments, R³ is a 5-membered heteroaryl, optionally substituted with 1 or 2 substituents independently selected from C₁₋₆ alkyl, C₁₋₄ haloalkyl, C₁₋₆ alkylcarbonyl, CN, halo, C₁₋₆ alkoxy, 4-6 membered heterocycloalkyl (e.g., tetrahydrofuranyl), and C₁₋₆ alkoxy-C₁₋₆ alkyl.

In some embodiments, the heteroaryl of R³ is selected from thiophenyl and pyrazolyl.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (III) is selected from any one of the following compounds:

TABLE III

| | |
|---|---|
| BCH- 136A | 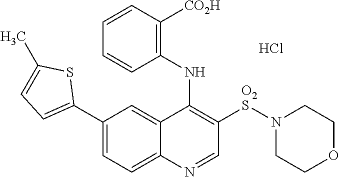 [SMILES: Cc1ccc(-c2ccc3ncc(S(=O)(=O)N4CCOCC4)c(Nc4ccccc4C(=O)O)c3c2)s1 ; Cl] |
| BCH- 137A | 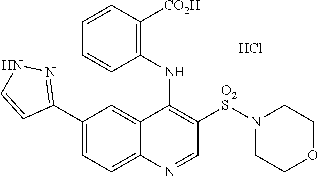 [SMILES: O=C(O)c1ccccc1Nc1c(S(=O)(=O)N2CCOCC2)cnc2ccc(-c3cc[nH]n3)cc12 ; Cl] |
| BCH- 138A | 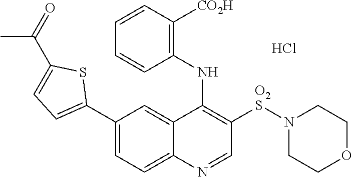 [SMILES: CC(=O)c1ccc(-c2ccc3ncc(S(=O)(=O)N4CCOCC4)c(Nc4ccccc4C(=O)O)c3c2)s1 ; Cl] |
| BCH- 139A | 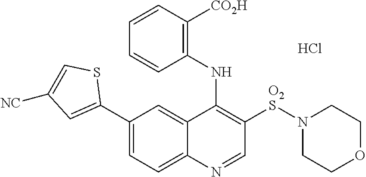 [SMILES: N#Cc1csc(-c2ccc3ncc(S(=O)(=O)N4CCOCC4)c(Nc4ccccc4C(=O)O)c3c2)c1 ; Cl] |
| BCH- 140A | 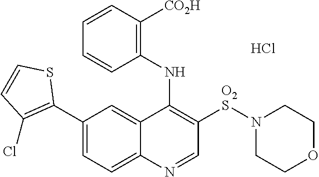 [SMILES: O=C(O)c1ccccc1Nc1c(S(=O)(=O)N2CCOCC2)cnc2ccc(-c3sccc3Cl)cc12 ; Cl] |
| BCH- 141A | 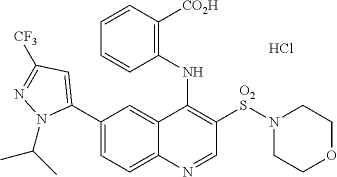 [SMILES: CC(C)n1nc(C(F)(F)F)cc1-c1ccc2ncc(S(=O)(=O)N3CCOCC3)c(Nc3ccccc3C(=O)O)c2c1 ; Cl] |
| BCH- 145A | 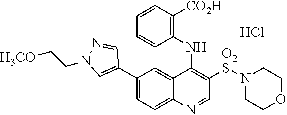 [SMILES: COCCn1cc(-c2ccc3ncc(S(=O)(=O)N4CCOCC4)c(Nc4ccccc4C(=O)O)c3c2)cn1 ; Cl] |
| BCH- 148A-R | 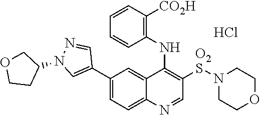 |

or a pharmaceutically acceptable salt thereof.

In some embodiments, the present disclosure provides a compound of Formula (IV):

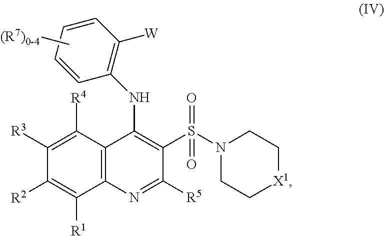

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
R³ is selected from pyridinyl and pyrimidinyl, each of which is optionally substituted with 1, 2, or 3 substituents independently selected from C₁₋₆ alkyl, C₁₋₄ haloalkyl, C₁₋₆ alkylcarbonyl, CN, OH, halo, C₁₋₆ alkoxy, C₁₋₆ alkoxy-C₁₋₆ alkyl, C₆₋₁₀ aryl, C₆₋₁₀ aryloxy, NO₂, C₁₋₆ haloalkoxy, cyano-C₁₋₃ alkylene, C₃₋₁₀ cycloalkyl, 4-6 membered heterocycloalkyl, amino, C₁₋₆ alkylamino, di(C₁₋₆ alkyl)amino, carboxy, C₁₋₆ alkylsulfonyl, C₁₋₆ alkoxycarbonyl, carbamyl, C₁₋₆ alkylcarbamyl, and di(C₁₋₆ alkyl)carbamyl;
W is selected from C(O)OR⁸ and a carboxylic acid bioisostere;
R⁸ is selected from H and C₁₋₆ alkyl; and
each R⁷ is independently selected from halo, C₁₋₃ alkyl, C₁₋₃ haloalkyl, C₁₋₃ haloalkoxy, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I))

In some embodiments, the compound of Formula (IV) has formula:

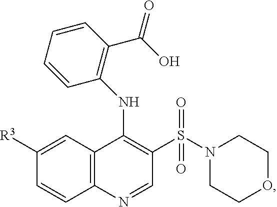

or a pharmaceutically acceptable salt thereof.

In some embodiments, R³ is selected from pyridinyl and pyrimidinyl, each of which is optionally substituted with 1, 2, or 3 substituents independently selected from C₁₋₆ alkyl, C₁₋₄ haloalkyl, CN, C₁₋₆ alkoxy, C₆₋₁₀ aryloxy, C₃₋₁₀ cycloalkyl, 4-6 membered heterocycloalkyl, amino, C₁₋₆ alkylamino, di(C₁₋₆ alkyl)amino, C₁₋₆ alkylsulfonyl, and C₁₋₆ alkylcarbamyl.

In some embodiments, R³ is pyridinyl, optionally substituted with 1 or 2 substituents independently selected from C₁₋₆ alkyl, C₁₋₄ haloalkyl, CN, C₁₋₆ alkoxy, C₆₋₁₀ aryloxy, C₃₋₁₀ cycloalkyl, 4-6 membered heterocycloalkyl, amino, C₁₋₆ alkylamino, di(C₁₋₆ alkyl)amino, C₁₋₆ alkylsulfonyl, and C₁₋₆ alkylcarbamyl.

In some embodiments, R³ is pyrimidinyl, optionally substituted with 1 or 2 substituents independently selected from C₁₋₆ alkyl, C₁₋₄ haloalkyl, CN, C₁₋₆ alkoxy, C₆₋₁₀ aryloxy, C₃₋₁₀ cycloalkyl, 4-6 membered heterocycloalkyl, amino, C₁₋₆ alkylamino, di(C₁₋₆ alkyl)amino, C₁₋₆ alkylsulfonyl, and C₁₋₆ alkylcarbamyl.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of formula (IV) is selected from any one of the following compounds:

TABLE IV

| | |
|---|---|
| BCH- 142A | 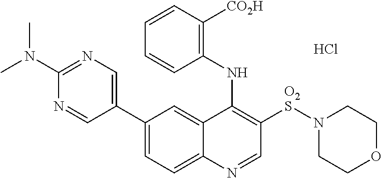 [SMILES: CN(C)c1ncc(-c2ccc3ncc(S(=O)(=O)N4CCOCC4)c(Nc4ccccc4C(=O)O)c3c2)cn1 ; Cl] |
| BCH- 143A | 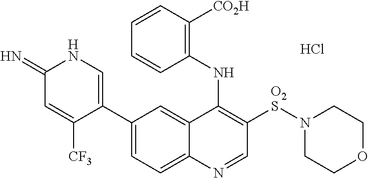 [SMILES: N=c1cc(C(F)(F)F)c(-c2ccc3ncc(S(=O)(=O)N4CCOCC4)c(Nc4ccccc4C(=O)O)c3c2)c[nH]1 ; Cl] |
| BCH- 150A | 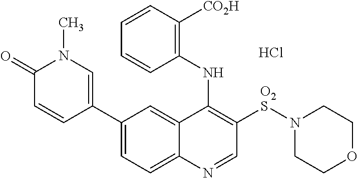 [SMILES: Cn1cc(-c2ccc3ncc(S(=O)(=O)N4CCOCC4)c(Nc4ccccc4C(=O)O)c3c2)ccc1=O ; Cl] |
| BCH- 151A | 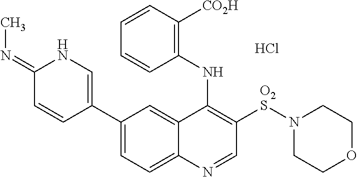 [SMILES: C/N=c1/ccc(-c2ccc3ncc(S(=O)(=O)N4CCOCC4)c(Nc4ccccc4C(=O)O)c3c2)c[nH]1 ; Cl] |
| BCH- 152A | 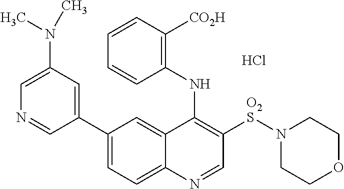 [SMILES: CN(C)c1cncc(-c2ccc3ncc(S(=O)(=O)N4CCOCC4)c(Nc4ccccc4C(=O)O)c3c2)c1 ; Cl] |
| BCH- 153A | 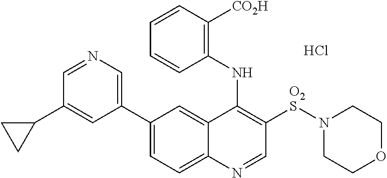 [SMILES: O=C(O)c1ccccc1Nc1c(S(=O)(=O)N2CCOCC2)cnc2ccc(-c3cncc(C4CC4)c3)cc12 ; Cl] |
| BCH- 154A | 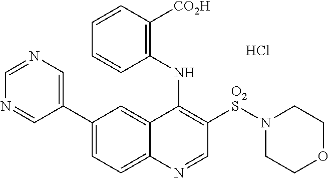 [SMILES: O=C(O)c1ccccc1Nc1c(S(=O)(=O)N2CCOCC2)cnc2ccc(-c3cncnc3)cc12 ; Cl] |
| BCH- 157A | 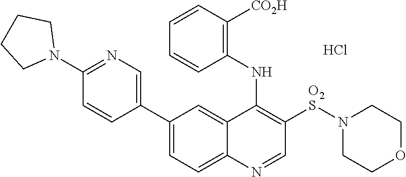 [SMILES: O=C(O)c1ccccc1Nc1c(S(=O)(=O)N2CCOCC2)cnc2ccc(-c3ccc(N4CCCC4)nc3)cc12 ; Cl] |
| BCH- 158A | 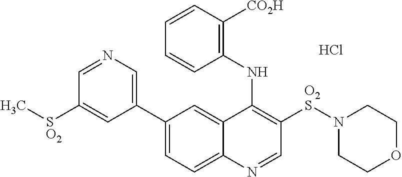 [SMILES: CS(=O)(=O)c1cncc(-c2ccc3ncc(S(=O)(=O)N4CCOCC4)c(Nc4ccccc4C(=O)O)c3c2)c1 ; Cl] |
| BCH- 161A | 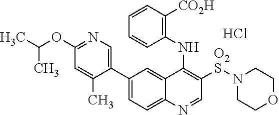 [SMILES: Cc1cc(OC(C)C)ncc1-c1ccc2ncc(S(=O)(=O)N3CCOCC3)c(Nc3ccccc3C(=O)O)c2c1 ; Cl] |
| BCH- 162A | 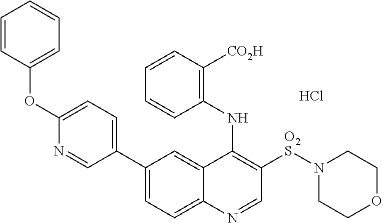 [SMILES: O=C(O)c1ccccc1Nc1c(S(=O)(=O)N2CCOCC2)cnc2ccc(-c3ccc(Oc4ccccc4)nc3)cc12 ; Cl] |
| BCH- 163A | 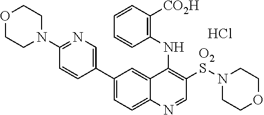 [SMILES: O=C(O)c1ccccc1Nc1c(S(=O)(=O)N2CCOCC2)cnc2ccc(-c3ccc(N4CCOCC4)nc3)cc12 ; Cl] |
| BCH- 164A | 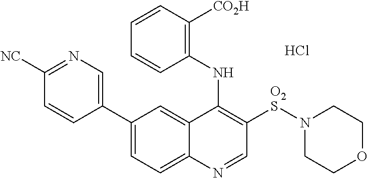 [SMILES: N#Cc1ccc(-c2ccc3ncc(S(=O)(=O)N4CCOCC4)c(Nc4ccccc4C(=O)O)c3c2)cn1 ; Cl] |
| BCH- 165A | 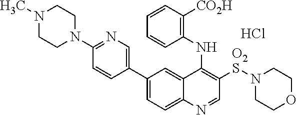 [SMILES: CN1CCN(c2ccc(-c3ccc4ncc(S(=O)(=O)N5CCOCC5)c(Nc5ccccc5C(=O)O)c4c3)cn2)CC1 ; Cl] |
| BCH- 166A | 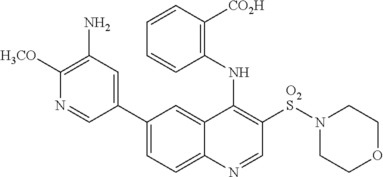 [SMILES: COc1ncc(-c2ccc3ncc(S(=O)(=O)N4CCOCC4)c(Nc4ccccc4C(=O)O)c3c2)cc1N] |
| BCH- 167A | 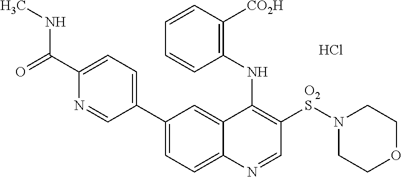 [SMILES: CNC(=O)c1ccc(-c2ccc3ncc(S(=O)(=O)N4CCOCC4)c(Nc4ccccc4C(=O)O)c3c2)cn1 ; Cl] |
| BCH- 168A | 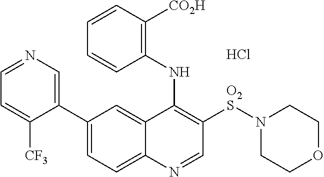 [SMILES: O=C(O)c1ccccc1Nc1c(S(=O)(=O)N2CCOCC2)cnc2ccc(-c3cnccc3C(F)(F)F)cc12 ; Cl] |
| BCH- 169A | 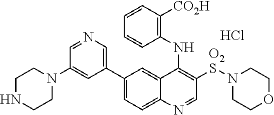 [SMILES: O=C(O)c1ccccc1Nc1c(S(=O)(=O)N2CCOCC2)cnc2ccc(-c3cncc(N4CCNCC4)c3)cc12 ; Cl] |

In some embodiments, the present disclosure provides a compound of Formula (V):

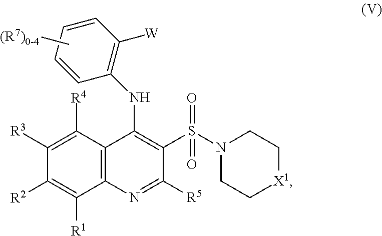

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ a 9 to 10-membered heteroaryl selected from the group consisting of:

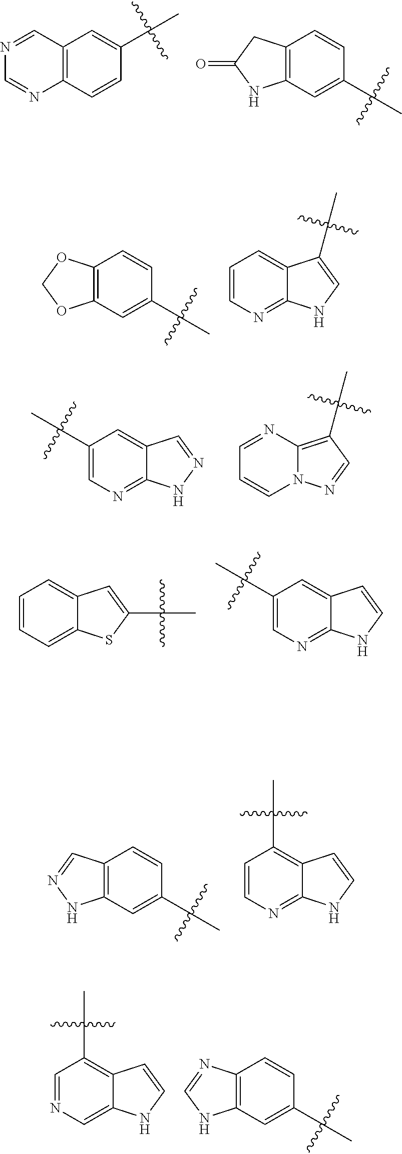

each of which is optionally substituted with 1, 2, or 3 substituents independently selected from C₁₋₆ alkyl, C₁₋₄ haloalkyl, C₁₋₆ alkylcarbonyl, CN, OH, halo, C₁₋₆ alkoxy, C₁₋₆ alkoxy-C₁₋₆ alkyl, C₆₋₁₀ aryl, C₆₋₁₀ aryloxy, NO₂, C₁₋₆ haloalkoxy, cyano-C₁₋₃ alkylene, C₃₋₁₀ cycloalkyl, 4-6 membered heterocycloalkyl, amino, C₁₋₆ alkylamino, di(C₁₋₆ alkyl)amino, carboxy, C₁₋₆ alkylsulfonyl, C₆₋₁₀ arylsulfonyl, C₁₋₆ alkoxycarbonyl, carbamyl, C₁₋₆ alkylcarbamyl, and di(C₁₋₆ alkyl)carbamyl; and
each R⁷ is selected from halo, C₁₋₃ alkyl, C₁₋₃ haloalkyl, C₁₋₃ haloalkoxy, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl. In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (IV) has formula:

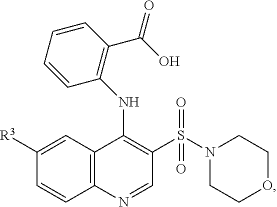

or a pharmaceutically acceptable salt thereof.

In some embodiments, R³ a 9 to 10-membered heteroaryl selected from the group consisting of:

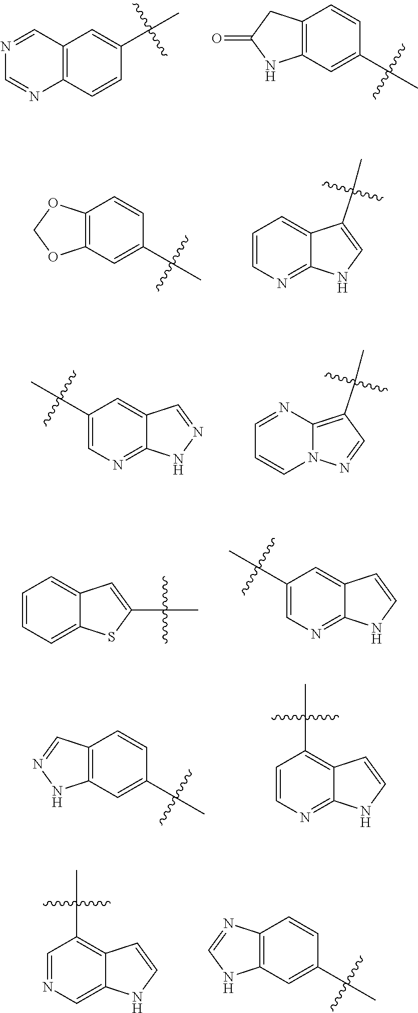

each of which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 10-membered heteroaryl group of formula:

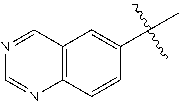 [SMILES: *c1ccc2ncncc2c1]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

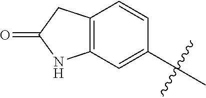 [SMILES: *c1ccc2c(c1)NC(=O)C2]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

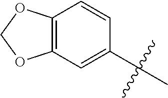 [SMILES: *c1ccc2c(c1)OCO2]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

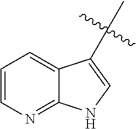 [SMILES: *c1c[nH]c2ncccc12]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

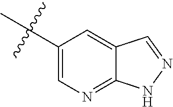 [SMILES: *c1cnc2[nH]ncc2c1]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

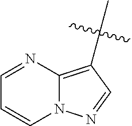 [SMILES: *c1cnn2cccnc12]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

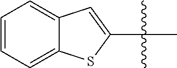 [SMILES: *c1cc2ccccc2s1]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

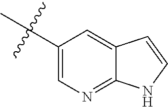 [SMILES: *c1cnc2[nH]ccc2c1]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

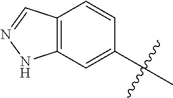 [SMILES: *c1ccc2cn[nH]c2c1]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

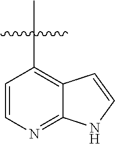 [SMILES: *c1ccnc2[nH]ccc12]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

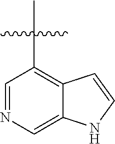 [SMILES: *c1cncc2[nH]ccc12]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, R³ a 9-membered heteroaryl group of formula:

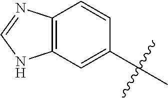 [SMILES: *c1ccc2nc[nH]c2c1]

which is optionally substituted with 1 or 2, substituents independently selected from C₁₋₆ alkyl, C₁₋₄ haloalkyl, halo, C₁₋₆ alkylamino, and C₆₋₁₀ arylsulfonyl.

In some embodiments, each R⁷ is independently selected from halo, C₁₋₃ alkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (V) is selected from any one of the following compounds:

TABLE V

| | |
|---|---|
| BCH- 144A | 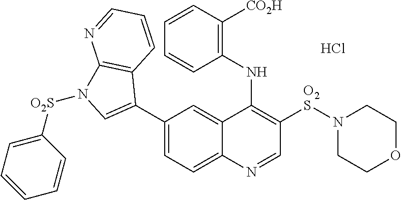 [SMILES: O=C(O)c1ccccc1Nc1c(S(=O)(=O)N2CCOCC2)cnc2ccc(-c3cn(S(=O)(=O)c4ccccc4)c4ncccc34)cc12 ; Cl] |
| BCH- 146A- Cl | 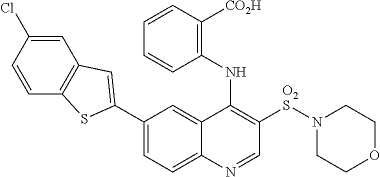 [SMILES: O=C(O)c1ccccc1Nc1c(S(=O)(=O)N2CCOCC2)cnc2ccc(-c3cc4cc(Cl)ccc4s3)cc12] |
| BCH- 147A | 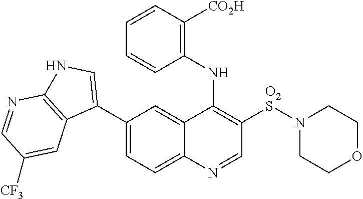 [SMILES: O=C(O)c1ccccc1Nc1c(S(=O)(=O)N2CCOCC2)cnc2ccc(-c3c[nH]c4ncc(C(F)(F)F)cc34)cc12] |
| BCH- 149A | 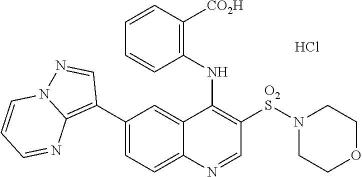 [SMILES: O=C(O)c1ccccc1Nc1c(S(=O)(=O)N2CCOCC2)cnc2ccc(-c3cnn4cccnc34)cc12 ; Cl] |
| BCH- 155A | 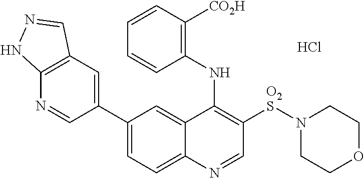 [SMILES: O=C(O)c1ccccc1Nc1c(S(=O)(=O)N2CCOCC2)cnc2ccc(-c3cnc4[nH]ncc4c3)cc12 ; Cl] |
| BCH- 156A | 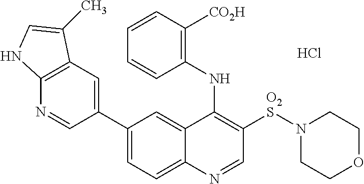 [SMILES: Cc1c[nH]c2ncc(-c3ccc4ncc(S(=O)(=O)N5CCOCC5)c(Nc5ccccc5C(=O)O)c4c3)cc12 ; Cl] |
| BCH- 159A | 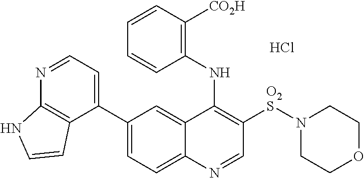 [SMILES: O=C(O)c1ccccc1Nc1c(S(=O)(=O)N2CCOCC2)cnc2ccc(-c3ccnc4[nH]ccc34)cc12 ; Cl] |
| BCH- 160A | 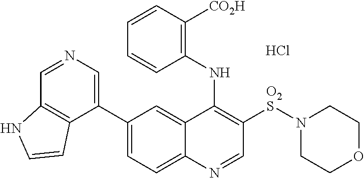 [SMILES: O=C(O)c1ccccc1Nc1c(S(=O)(=O)N2CCOCC2)cnc2ccc(-c3cncc4[nH]ccc34)cc12 ; Cl] |
| BCH- 170A | 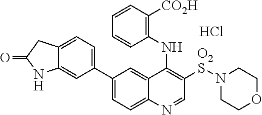 [SMILES: O=C1Cc2ccc(-c3ccc4ncc(S(=O)(=O)N5CCOCC5)c(Nc5ccccc5C(=O)O)c4c3)cc2N1 ; Cl] |
| BCH- 171A | 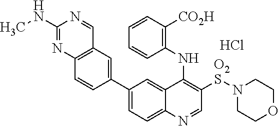 [SMILES: CNc1ncc2cc(-c3ccc4ncc(S(=O)(=O)N5CCOCC5)c(Nc5ccccc5C(=O)O)c4c3)ccc2n1 ; Cl] |
| BCH- 175A | 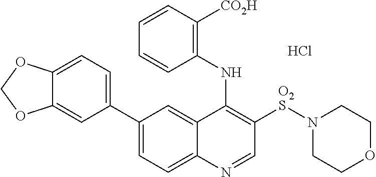 [SMILES: O=C(O)c1ccccc1Nc1c(S(=O)(=O)N2CCOCC2)cnc2ccc(-c3ccc4c(c3)OCO4)cc12 ; Cl] |
| BCH- 177A | 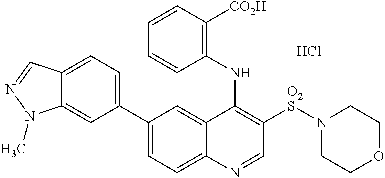 [SMILES: Cn1ncc2ccc(-c3ccc4ncc(S(=O)(=O)N5CCOCC5)c(Nc5ccccc5C(=O)O)c4c3)cc21 ; Cl] |
| BCH- 181A | 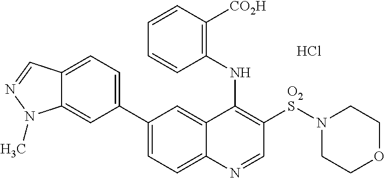 [SMILES: Cn1ncc2ccc(-c3ccc4ncc(S(=O)(=O)N5CCOCC5)c(Nc5ccccc5C(=O)O)c4c3)cc21 ; Cl] |
| BCH- 182A | 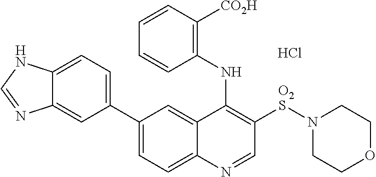 [SMILES: O=C(O)c1ccccc1Nc1c(S(=O)(=O)N2CCOCC2)cnc2ccc(-c3ccc4[nH]cnc4c3)cc12 ; Cl] |

In some embodiments, the present disclosure provides a compound of Formula (VI)

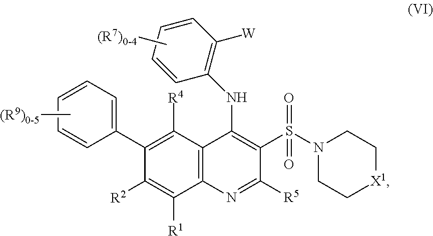

or a pharmaceutically acceptable salt thereof, wherein.
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
each R⁹ is independently selected from C₁₋₆ alkyl, C₁₋₄ haloalkyl, C₁₋₆ alkylcarbonyl, CN, OH, halo, C₁₋₆ alkoxy, C₁₋₆ alkoxy-C₁₋₆ alkyl, C₆₋₁₀ aryl, C₆₋₁₀ aryloxy, NO₂, C₁₋₆ haloalkoxy, cyano-C₁₋₃ alkylene, C₃₋₁₀ cycloalkyl, 4-6 membered heterocycloalkyl, 5-6 membered heteroaryl, amino, C₁₋₆ alkylamino, di(C₁₋₆ alkyl)amino, carboxy, C₁₋₆ alkylsulfonyl, C₆₋₁₀ arylsulfonyl, 5-6 membered heterocycloalkylsulfonyl, C₁₋₆ alkoxycarbonyl, carbamyl, C₁₋₆ alkylcarbamyl, di(C₁₋₆ alkyl)carbamyl, C₁₋₆ alkylsulfonylamino, wherein said 6 membered heterocycloalkyl and 5-6 membered heteroaryl are each optionally substituted with 1 or 2 substituents independently selected from C₁₋₆ alkyl, C₁₋₄ haloalkyl, C₁₋₆ alkoxy, and C₁₋₄ haloalkoxy; and
each R⁷ is selected from halo, C₁₋₃ alkyl, C₁₋₃ haloalkyl, C₁₋₃ haloalkoxy, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (VI) has formula:

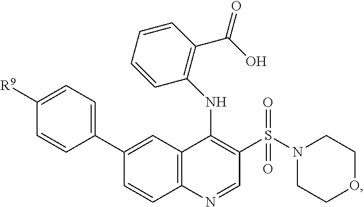

or a pharmaceutically acceptable salt thereof.

In some embodiments, R⁹ is selected from 5-6 membered heteroaryl, di(C₁₋₆ alkyl)amino, carboxy, 5-6 membered heterocycloalkylsulfonyl, di(C₁₋₆ alkyl)carbamyl, and C₁₋₆ alkylsulfonylamino, wherein said 5-6 membered heteroaryl is optionally substituted with C₁₋₆ alkyl.

In some embodiments, R⁷ is selected from halo, C₁₋₃ alkyl, and C₁₋₃ alkoxy. In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (VI) is selected from any one of the following compounds:

TABLE VI

| | |
|---|---|
| BCH- 172A | 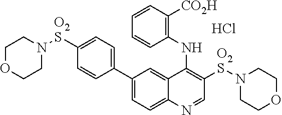 [SMILES: O=C(O)c1ccccc1Nc1c(S(=O)(=O)N2CCOCC2)cnc2ccc(-c3ccc(S(=O)(=O)N4CCOCC4)cc3)cc12 ; Cl] |
| BCH- 173A | 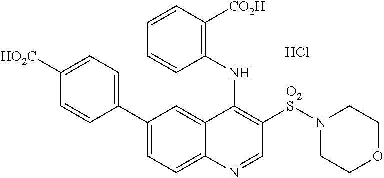 [SMILES: O=C(O)c1ccc(-c2ccc3ncc(S(=O)(=O)N4CCOCC4)c(Nc4ccccc4C(=O)O)c3c2)cc1 ; Cl] |
| BCH- 174A | 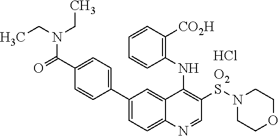 [SMILES: CCN(CC)C(=O)c1ccc(-c2ccc3ncc(S(=O)(=O)N4CCOCC4)c(Nc4ccccc4C(=O)O)c3c2)cc1 ; Cl] |
| BCH- 176A | 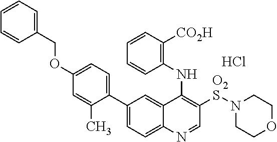 [SMILES: Cc1cc(OCc2ccccc2)ccc1-c1ccc2ncc(S(=O)(=O)N3CCOCC3)c(Nc3ccccc3C(=O)O)c2c1 ; Cl] |
| BCH- 178A | 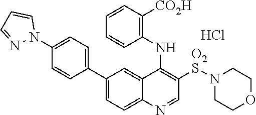 [SMILES: O=C(O)c1ccccc1Nc1c(S(=O)(=O)N2CCOCC2)cnc2ccc(-c3ccc(-n4cccn4)cc3)cc12 ; Cl] |
| BCH- 179A | 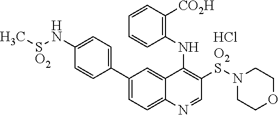 [SMILES: CS(=O)(=O)Nc1ccc(-c2ccc3ncc(S(=O)(=O)N4CCOCC4)c(Nc4ccccc4C(=O)O)c3c2)cc1 ; Cl] |
| BCH- 180A | 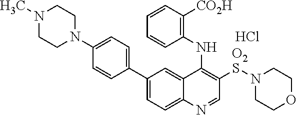 [SMILES: CN1CCN(c2ccc(-c3ccc4ncc(S(=O)(=O)N5CCOCC5)c(Nc5ccccc5C(=O)O)c4c3)cc2)CC1 ; Cl] |
| BCH- 183A | 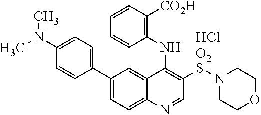 [SMILES: CN(C)c1ccc(-c2ccc3ncc(S(=O)(=O)N4CCOCC4)c(Nc4ccccc4C(=O)O)c3c2)cc1 ; Cl] |

or a pharmaceutically acceptable salt thereof.

In some embodiments, the present disclosure provides a compound of formula (VII):

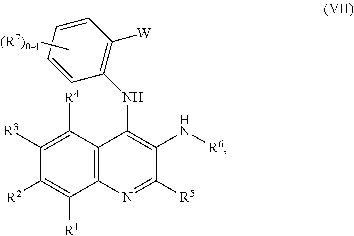

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is selected from 5-6 membered heterocycloalkyl, C₄₋₆ cycloalkyl, and a 5-6 membered heteroaryl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxy carbonyl;
R³ is halo; and
each R⁷ is independently selected from halo, C₁₋₃ alkyl, C₁₋₃ haloalkyl, C₁₋₃ haloalkoxy, and C₁₋₃ alkoxy.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (VII) has formula:

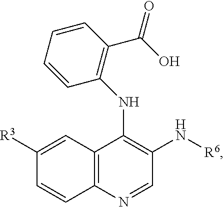

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁶ is selected from tetrahydropyranyl, cyclohexyl, piperidinyl, and 1,1-dioxo tetrahydro-2H-thiopyranyl, pyrimidinyl, oxazolyl, thioxazolyl, and thiazolyl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl.

In some embodiments, R⁶ is selected from tetrahydropyranyl, cyclohexyl, piperidinyl, and 1,1-dioxo tetrahydro-2H-thiopyranyl, pyrimidinyl, oxazolyl, thioxazolyl. 1,3,4-oxadiazolyl, and thiazolyl, each of which is optionally substituted with halo.

In some embodiments, R⁷ is selected from halo, C₁₋₃ alkyl, and C₁₋₃ alkoxy. In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (VII) is selected from any one of the following compounds:

TABLE VII

| | |
|---|---|
| BCH-184A | 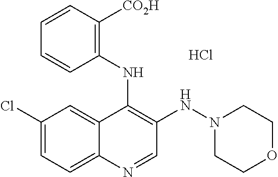 [SMILES: O=C(O)c1ccccc1Nc1c(NN2CCOCC2)cnc2ccc(Cl)cc12 ; Cl] |
| BCH-185A | 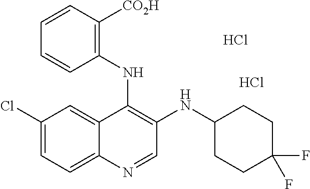 [SMILES: O=C(O)c1ccccc1Nc1c(NC2CCC(F)(F)CC2)cnc2ccc(Cl)cc12 ; Cl ; Cl] |
| BCH-186A | 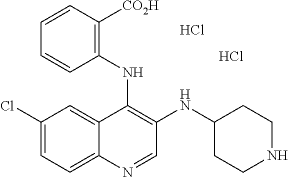 [SMILES: O=C(O)c1ccccc1Nc1c(NC2CCNCC2)cnc2ccc(Cl)cc12 ; Cl ; Cl] |
| BCH-188A | 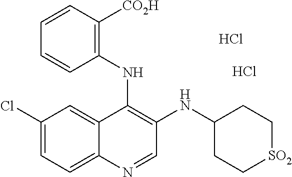 [SMILES: O=C(O)c1ccccc1Nc1c(NC2CCS(=O)(=O)CC2)cnc2ccc(Cl)cc12 ; Cl ; Cl] |
| BCH-191A | 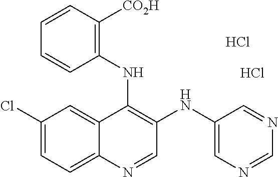 [SMILES: O=C(O)c1ccccc1Nc1c(Nc2cncnc2)cnc2ccc(Cl)cc12 ; Cl ; Cl] |
| 192A | 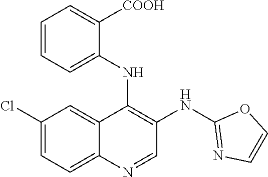 [SMILES: O=C(O)c1ccccc1Nc1c(Nc2ncco2)cnc2ccc(Cl)cc12] |
| 193A | 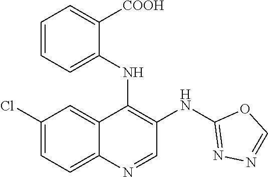 [SMILES: O=C(O)c1ccccc1Nc1c(Nc2nnco2)cnc2ccc(Cl)cc12] |
| 195A | 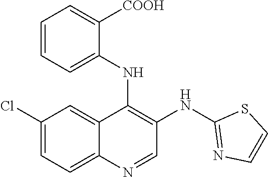 [SMILES: O=C(O)c1ccccc1Nc1c(Nc2nccs2)cnc2ccc(Cl)cc12] |

or a pharmaceutically acceptable salt thereof.

In some embodiments, the present disclosure provides a compound of formula (VIII):

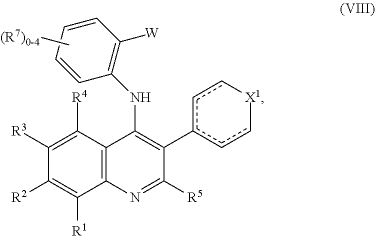

or a pharmaceutically acceptable salt thereof, wherein:
X¹ is selected from O, S, CF₂, C═O, CHCl, CHF, CCl₂, C═N—OH, NH, NCH₃, Si(OH)₂, SO₂, and cyclopropylidene;
each [symbol] is independently a single bond or a double bond, provided that no more than two of [symbol] are double bonds;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

In some embodiments, X¹ is selected from O, S, CF₂, CHCl, CCl₂, NH, NCH₃, Si(OH)₂, SO₂, and cyclopropylidene.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo:

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, X¹ is O. In some embodiments, X¹ is S. In some embodiments, X¹ is CF₂. In some embodiments, X¹ is CHCl. In some embodiments, X¹ is CCl₂. In some embodiments, X¹ is NH. In some embodiments, X¹ is NCH₃. In some embodiments, X¹ is Si(OH)₂. In some embodiments, X¹ is SO₂. In some embodiments, X¹ is cyclopropylidene. In some embodiments, X¹ is C═O. In some embodiments, X¹ is CHF. In some embodiments, X¹ is C═N—OH.

In some embodiments, the compound of Formula (VIII) has formula:

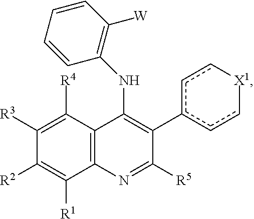

or a pharmaceutically acceptable salt thereof.

In some embodiments, the compound of Formula (VIII) has formula:

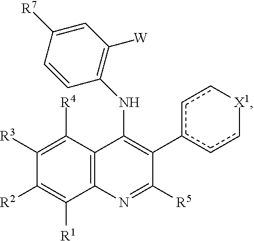

or a pharmaceutically acceptable salt thereof.

In some embodiments, the compound of Formula (VIII) has formula:

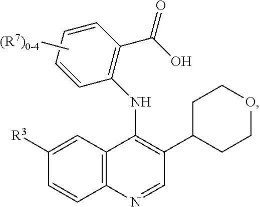

or a pharmaceutically acceptable salt thereof.

In some embodiments, the compound of Formula (VIII) has formula:

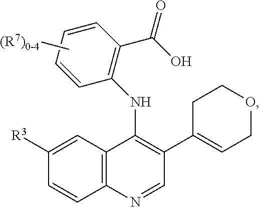

or a pharmaceutically acceptable salt thereof.

In some embodiments, the compound of Formula (VIII) has formula:

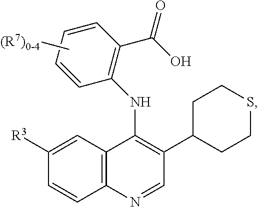

or a pharmaceutically acceptable salt thereof.

In some embodiments, the compound of Formula (VIII) has formula:

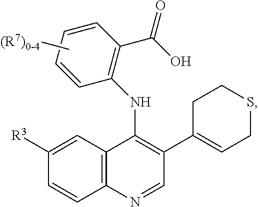

or a pharmaceutically acceptable salt thereof.

In some embodiments, the compound of Formula (VIII) has formula:

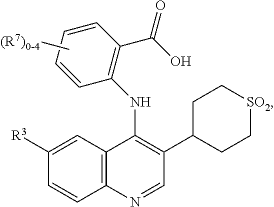

or a pharmaceutically acceptable salt thereof.

In some embodiments, the compound of Formula (VIII) has formula:

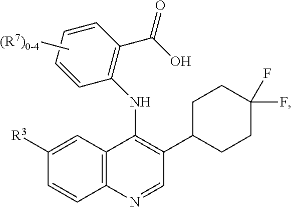

or a pharmaceutically acceptable salt thereof.

In some embodiments, the compound of Formula (VIII) has formula:

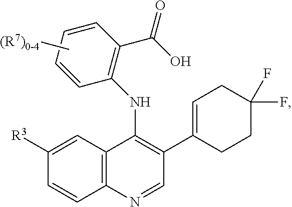

or a pharmaceutically acceptable salt thereof.

In some embodiments, the compound of Formula (VIII) has formula:

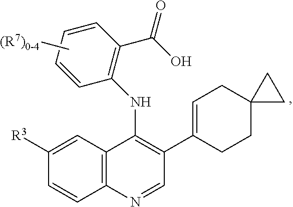

or a pharmaceutically acceptable salt thereof.

In some embodiments, the compound of Formula (VIII) has formula:

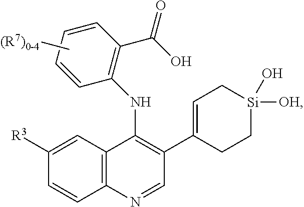

or a pharmaceutically acceptable salt thereof.

In some embodiments, the compound of Formula (VIII) has formula:

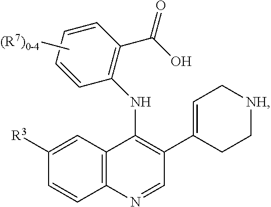

or a pharmaceutically acceptable salt thereof.

In some embodiments, the compound of Formula (VIII) has formula:

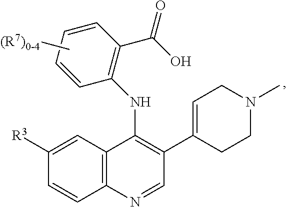

or a pharmaceutically acceptable salt thereof.

In some embodiments, the compound of Formula (VIII) has formula:

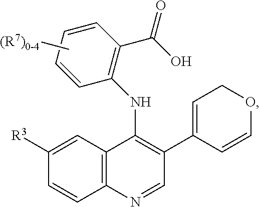

or a pharmaceutically acceptable salt thereof.

In some embodiments, the compound of Formula (VIII) has formula:

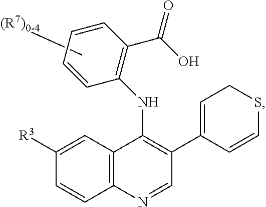

or a pharmaceutically acceptable salt thereof.

In some embodiments, the compound of Formula (VIII) has formula:

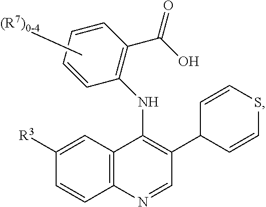

or a pharmaceutically acceptable salt thereof.

In some embodiments, the compound of Formula (VIII) has formula:

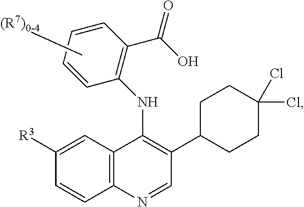

or a pharmaceutically acceptable salt thereof.

In some embodiments, the compound of Formula (VIII) has formula:

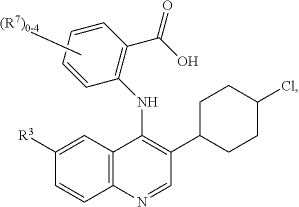

or a pharmaceutically acceptable salt thereof.

In some embodiments, the compound of Formula (VIII) has formula:

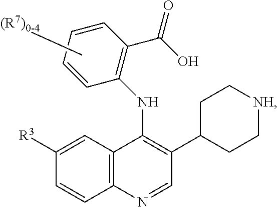

or a pharmaceutically acceptable salt thereof.

In some embodiments, the compound of Formula (VIII) has formula:

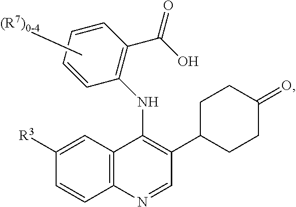

or a pharmaceutically acceptable salt thereof.

In some embodiments, the compound of Formula (VIII) has formula:

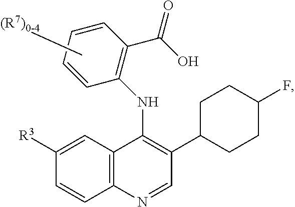

or a pharmaceutically acceptable salt thereof.

In some embodiments, the compound of Formula (VIII) has formula:

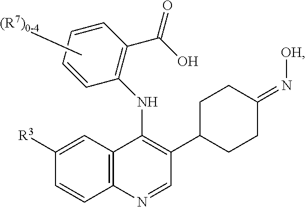

or a pharmaceutically acceptable salt thereof.

In some embodiments, R³ is selected from Cl, Br, and F. In some embodiments, R³ is Cl. In some embodiments, R³ is Br. In some embodiments, R³ is F.

In some embodiments, R⁷ is selected from halo, C₁₋₃ alkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (VIII) is selected from any one of the following compounds:

TABLE VIII

| | |
|---|---|
| BCH-204A-INT | 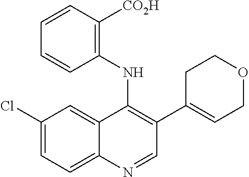 [SMILES: O=C(O)c1ccccc1Nc1c(C2=CCOCC2)cnc2ccc(Cl)cc12] |
| BCH-204A | 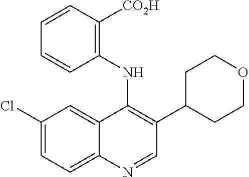 [SMILES: O=C(O)c1ccccc1Nc1c(C2CCOCC2)cnc2ccc(Cl)cc12] |
| BCH-205A | 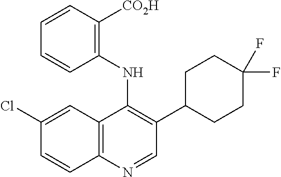 [SMILES: O=C(O)c1ccccc1Nc1c(C2CCC(F)(F)CC2)cnc2ccc(Cl)cc12] |
| BCH-205A-INT | 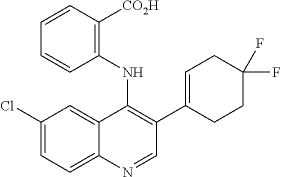 [SMILES: O=C(O)c1ccccc1Nc1c(C2=CCC(F)(F)CC2)cnc2ccc(Cl)cc12] |
| BCH-206A | 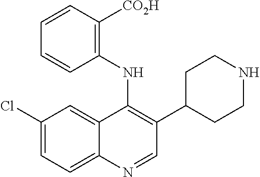 [SMILES: O=C(O)c1ccccc1Nc1c(C2CCNCC2)cnc2ccc(Cl)cc12] |
| BCH-206A-INT | 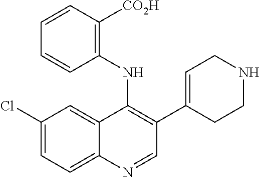 [SMILES: O=C(O)c1ccccc1Nc1c(C2=CCNCC2)cnc2ccc(Cl)cc12] |
| BCH-207A | 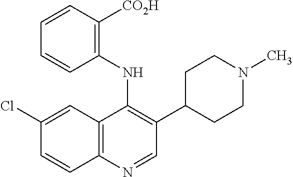 [SMILES: CN1CCC(c2cnc3ccc(Cl)cc3c2Nc2ccccc2C(=O)O)CC1] |
| BCH-207A-INT | 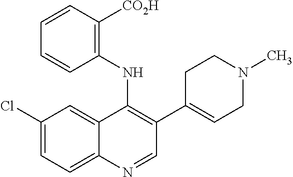 [SMILES: CN1CC=C(c2cnc3ccc(Cl)cc3c2Nc2ccccc2C(=O)O)CC1] |
| BCH-208A | 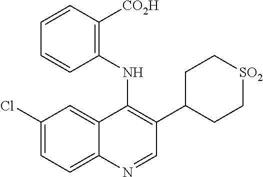 [SMILES: O=C(O)c1ccccc1Nc1c(C2CCS(=O)(=O)CC2)cnc2ccc(Cl)cc12] |
| BCH-208A-INT | 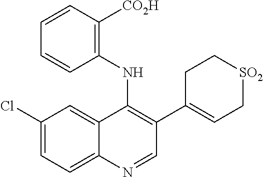 [SMILES: O=C(O)c1ccccc1Nc1c(C2=CCS(=O)(=O)CC2)cnc2ccc(Cl)cc12] |
| BCH-296A | 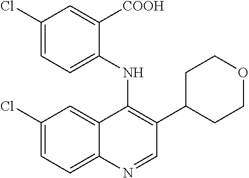 [SMILES: O=C(O)c1cc(Cl)ccc1Nc1c(C2CCOCC2)cnc2ccc(Cl)cc12] |
| BCH-297A | 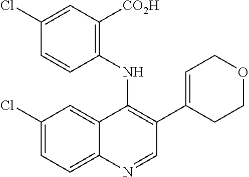 [SMILES: O=C(O)c1cc(Cl)ccc1Nc1c(C2=CCOCC2)cnc2ccc(Cl)cc12] |
| BCH-298A | 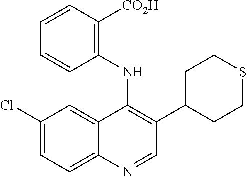 [SMILES: O=C(O)c1ccccc1Nc1c(C2CCSCC2)cnc2ccc(Cl)cc12] |
| BCH-299A | 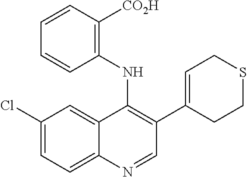 [SMILES: O=C(O)c1ccccc1Nc1c(C2=CCSCC2)cnc2ccc(Cl)cc12] |
| BCH-303A | 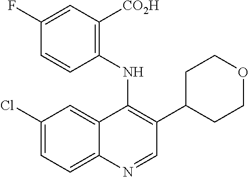 [SMILES: O=C(O)c1cc(F)ccc1Nc1c(C2CCOCC2)cnc2ccc(Cl)cc12] |
| BCH-304A | 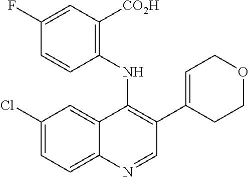 [SMILES: O=C(O)c1cc(F)ccc1Nc1c(C2=CCOCC2)cnc2ccc(Cl)cc12] |
| BCH-305A | 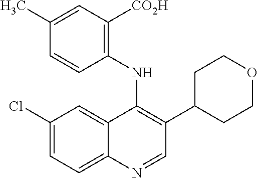 [SMILES: Cc1ccc(Nc2c(C3CCOCC3)cnc3ccc(Cl)cc23)c(C(=O)O)c1] |
| BCH-306A | 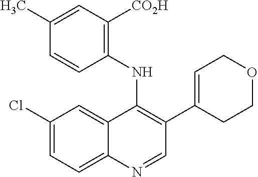 [SMILES: Cc1ccc(Nc2c(C3=CCOCC3)cnc3ccc(Cl)cc23)c(C(=O)O)c1] |
| BCH-307A | 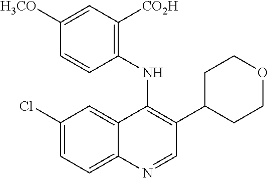 [SMILES: COc1ccc(Nc2c(C3CCOCC3)cnc3ccc(Cl)cc23)c(C(=O)O)c1] |
| BCH-308A | 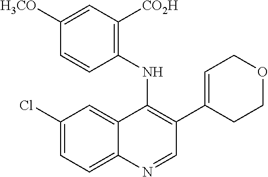 [SMILES: COc1ccc(Nc2c(C3=CCOCC3)cnc3ccc(Cl)cc23)c(C(=O)O)c1] |
| BCH-323A | 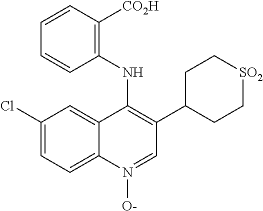 |
| BCH-341A | 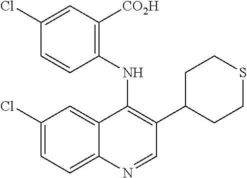 [SMILES: O=C(O)c1cc(Cl)ccc1Nc1c(C2CCSCC2)cnc2ccc(Cl)cc12] |
| BCH-342A | 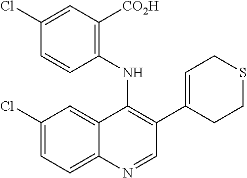 [SMILES: O=C(O)c1cc(Cl)ccc1Nc1c(C2=CCSCC2)cnc2ccc(Cl)cc12] |
| BCH-344A | 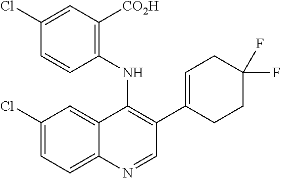 [SMILES: O=C(O)c1cc(Cl)ccc1Nc1c(C2=CCC(F)(F)CC2)cnc2ccc(Cl)cc12] |
| BCH-343A | 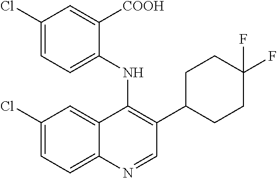 [SMILES: O=C(O)c1cc(Cl)ccc1Nc1c(C2CCC(F)(F)CC2)cnc2ccc(Cl)cc12] |
| | 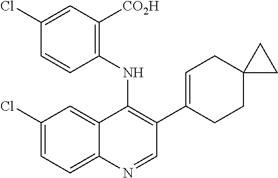 [SMILES: O=C(O)c1cc(Cl)ccc1Nc1c(C2=CCC3(CC2)CC3)cnc2ccc(Cl)cc12] |
| | 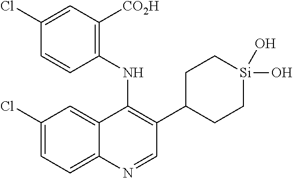 [SMILES: O=C(O)c1cc(Cl)ccc1Nc1c(C2CC[Si](O)(O)CC2)cnc2ccc(Cl)cc12] |
| BCH-367A | 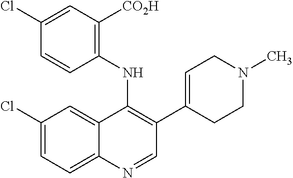 [SMILES: CN1CC=C(c2cnc3ccc(Cl)cc3c2Nc2ccc(Cl)cc2C(=O)O)CC1] |
| BCH-368A | 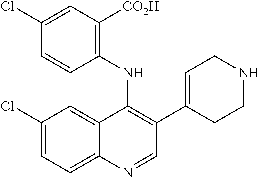 [SMILES: O=C(O)c1cc(Cl)ccc1Nc1c(C2=CCNCC2)cnc2ccc(Cl)cc12] |
| | 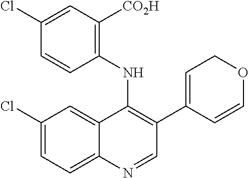 [SMILES: O=C(O)c1cc(Cl)ccc1Nc1c(C2=CCOC=C2)cnc2ccc(Cl)cc12] |
| | 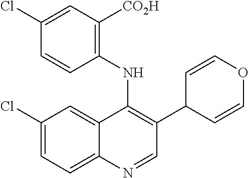 [SMILES: O=C(O)c1cc(Cl)ccc1Nc1c(C2C=COC=C2)cnc2ccc(Cl)cc12] |
| | 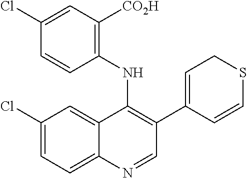 [SMILES: O=C(O)c1cc(Cl)ccc1Nc1c(C2=CCSC=C2)cnc2ccc(Cl)cc12] |
| | 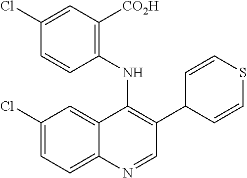 [SMILES: O=C(O)c1cc(Cl)ccc1Nc1c(C2C=CSC=C2)cnc2ccc(Cl)cc12] |
| | 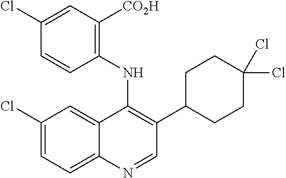 [SMILES: O=C(O)c1cc(Cl)ccc1Nc1c(C2CCC(Cl)(Cl)CC2)cnc2ccc(Cl)cc12] |
| | 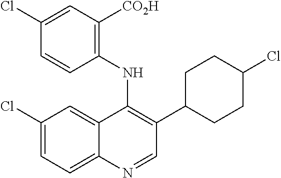 [SMILES: O=C(O)c1cc(Cl)ccc1Nc1c(C2CCC(Cl)CC2)cnc2ccc(Cl)cc12] |
| | 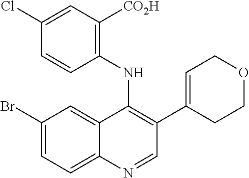 [SMILES: O=C(O)c1cc(Cl)ccc1Nc1c(C2=CCOCC2)cnc2ccc(Br)cc12] |
| | 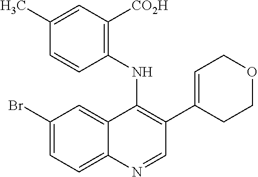 [SMILES: Cc1ccc(Nc2c(C3=CCOCC3)cnc3ccc(Br)cc23)c(C(=O)O)c1] |
| | 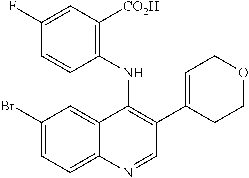 [SMILES: O=C(O)c1cc(F)ccc1Nc1c(C2=CCOCC2)cnc2ccc(Br)cc12] |
| | 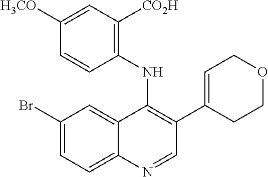 [SMILES: COc1ccc(Nc2c(C3=CCOCC3)cnc3ccc(Br)cc23)c(C(=O)O)c1] |
| | 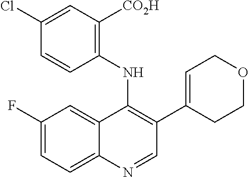 [SMILES: O=C(O)c1cc(Cl)ccc1Nc1c(C2=CCOCC2)cnc2ccc(F)cc12] |
| | 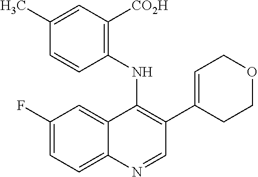 [SMILES: Cc1ccc(Nc2c(C3=CCOCC3)cnc3ccc(F)cc23)c(C(=O)O)c1] |
| | 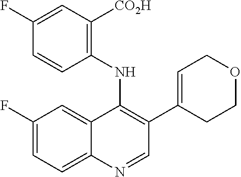 [SMILES: O=C(O)c1cc(F)ccc1Nc1c(C2=CCOCC2)cnc2ccc(F)cc12] |
| | 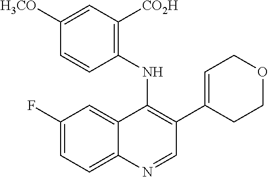 [SMILES: COc1ccc(Nc2c(C3=CCOCC3)cnc3ccc(F)cc23)c(C(=O)O)c1] |
| | 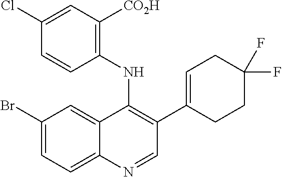 [SMILES: O=C(O)c1cc(Cl)ccc1Nc1c(C2=CCC(F)(F)CC2)cnc2ccc(Br)cc12] |
| | 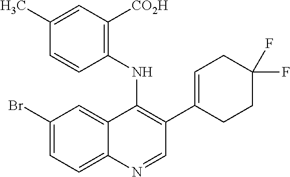 [SMILES: Cc1ccc(Nc2c(C3=CCC(F)(F)CC3)cnc3ccc(Br)cc23)c(C(=O)O)c1] |
| | 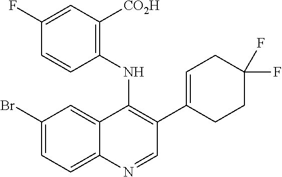 [SMILES: O=C(O)c1cc(F)ccc1Nc1c(C2=CCC(F)(F)CC2)cnc2ccc(Br)cc12] |
| | 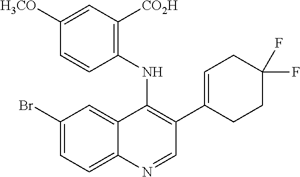 [SMILES: COc1ccc(Nc2c(C3=CCC(F)(F)CC3)cnc3ccc(Br)cc23)c(C(=O)O)c1] |
| | 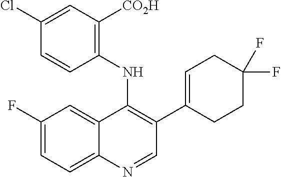 [SMILES: O=C(O)c1cc(Cl)ccc1Nc1c(C2=CCC(F)(F)CC2)cnc2ccc(F)cc12] |
| | 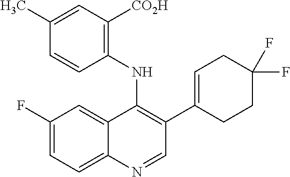 [SMILES: Cc1ccc(Nc2c(C3=CCC(F)(F)CC3)cnc3ccc(F)cc23)c(C(=O)O)c1] |
| | 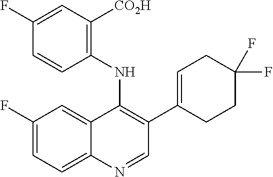 [SMILES: O=C(O)c1cc(F)ccc1Nc1c(C2=CCC(F)(F)CC2)cnc2ccc(F)cc12] |
| | 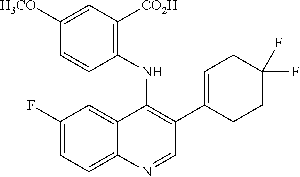 [SMILES: COc1ccc(Nc2c(C3=CCC(F)(F)CC3)cnc3ccc(F)cc23)c(C(=O)O)c1] |
| | 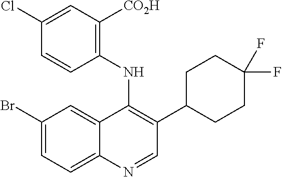 [SMILES: O=C(O)c1cc(Cl)ccc1Nc1c(C2CCC(F)(F)CC2)cnc2ccc(Br)cc12] |
| | 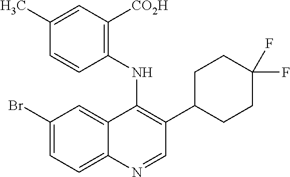 [SMILES: Cc1ccc(Nc2c(C3CCC(F)(F)CC3)cnc3ccc(Br)cc23)c(C(=O)O)c1] |
| | 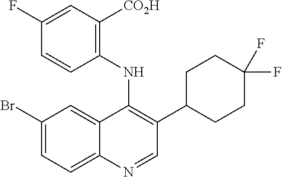 [SMILES: O=C(O)c1cc(F)ccc1Nc1c(C2CCC(F)(F)CC2)cnc2ccc(Br)cc12] |
| | 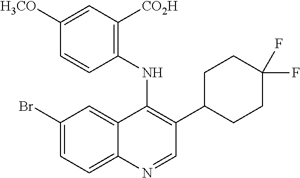 [SMILES: COc1ccc(Nc2c(C3CCC(F)(F)CC3)cnc3ccc(Br)cc23)c(C(=O)O)c1] |
| | 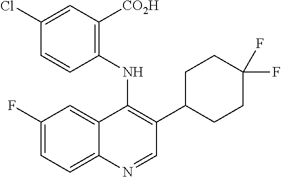 [SMILES: O=C(O)c1cc(Cl)ccc1Nc1c(C2CCC(F)(F)CC2)cnc2ccc(F)cc12] |
| | 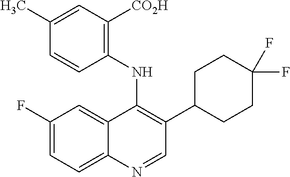 [SMILES: Cc1ccc(Nc2c(C3CCC(F)(F)CC3)cnc3ccc(F)cc23)c(C(=O)O)c1] |
| | 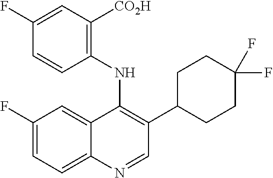 [SMILES: O=C(O)c1cc(F)ccc1Nc1c(C2CCC(F)(F)CC2)cnc2ccc(F)cc12] |
| | 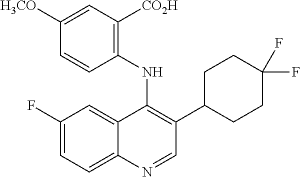 [SMILES: COc1ccc(Nc2c(C3CCC(F)(F)CC3)cnc3ccc(F)cc23)c(C(=O)O)c1] |
| | 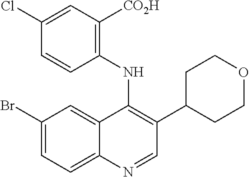 [SMILES: O=C(O)c1cc(Cl)ccc1Nc1c(C2CCOCC2)cnc2ccc(Br)cc12] |
| | 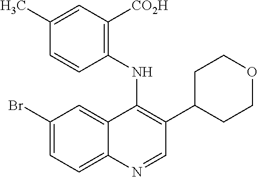 [SMILES: Cc1ccc(Nc2c(C3CCOCC3)cnc3ccc(Br)cc23)c(C(=O)O)c1] |
| | 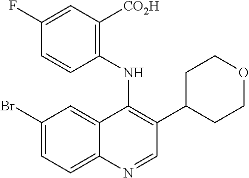 [SMILES: O=C(O)c1cc(F)ccc1Nc1c(C2CCOCC2)cnc2ccc(Br)cc12] |
| | 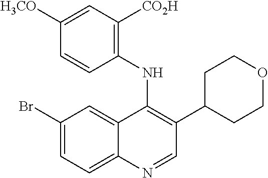 [SMILES: COc1ccc(Nc2c(C3CCOCC3)cnc3ccc(Br)cc23)c(C(=O)O)c1] |
| | 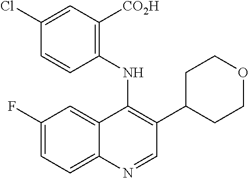 [SMILES: O=C(O)c1cc(Cl)ccc1Nc1c(C2CCOCC2)cnc2ccc(F)cc12] |
| | 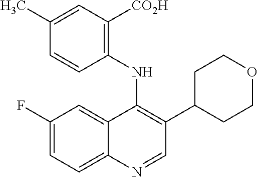 [SMILES: Cc1ccc(Nc2c(C3CCOCC3)cnc3ccc(F)cc23)c(C(=O)O)c1] |
| | 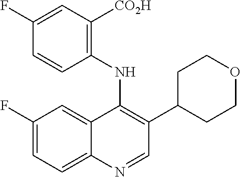 [SMILES: O=C(O)c1cc(F)ccc1Nc1c(C2CCOCC2)cnc2ccc(F)cc12] |
| | 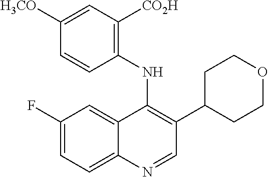 [SMILES: COc1ccc(Nc2c(C3CCOCC3)cnc3ccc(F)cc23)c(C(=O)O)c1] |
| | 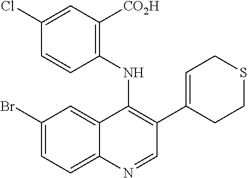 [SMILES: O=C(O)c1cc(Cl)ccc1Nc1c(C2=CCSCC2)cnc2ccc(Br)cc12] |
| | 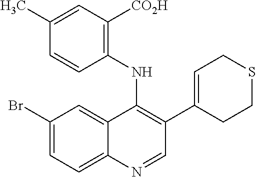 [SMILES: Cc1ccc(Nc2c(C3=CCSCC3)cnc3ccc(Br)cc23)c(C(=O)O)c1] |
| | 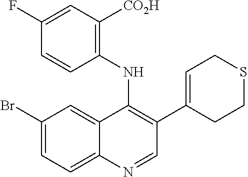 [SMILES: O=C(O)c1cc(F)ccc1Nc1c(C2=CCSCC2)cnc2ccc(Br)cc12] |
| | 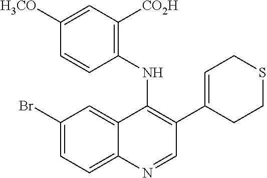 [SMILES: COc1ccc(Nc2c(C3=CCSCC3)cnc3ccc(Br)cc23)c(C(=O)O)c1] |
| | 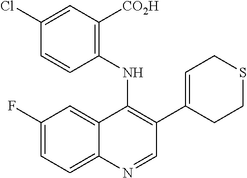 [SMILES: O=C(O)c1cc(Cl)ccc1Nc1c(C2=CCSCC2)cnc2ccc(F)cc12] |
| | 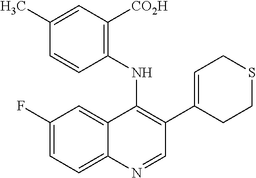 [SMILES: Cc1ccc(Nc2c(C3=CCSCC3)cnc3ccc(F)cc23)c(C(=O)O)c1] |
| | 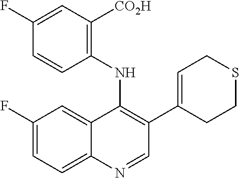 [SMILES: O=C(O)c1cc(F)ccc1Nc1c(C2=CCSCC2)cnc2ccc(F)cc12] |
| | 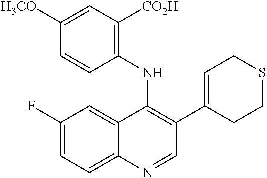 [SMILES: COc1ccc(Nc2c(C3=CCSCC3)cnc3ccc(F)cc23)c(C(=O)O)c1] |
| | 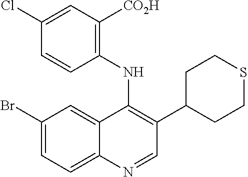 [SMILES: O=C(O)c1cc(Cl)ccc1Nc1c(C2CCSCC2)cnc2ccc(Br)cc12] |
| | 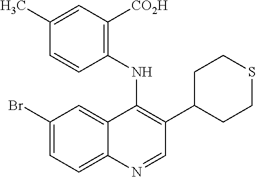 [SMILES: Cc1ccc(Nc2c(C3CCSCC3)cnc3ccc(Br)cc23)c(C(=O)O)c1] |
| | 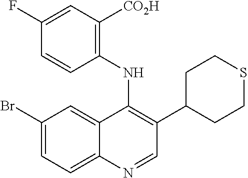 [SMILES: O=C(O)c1cc(F)ccc1Nc1c(C2CCSCC2)cnc2ccc(Br)cc12] |
| | 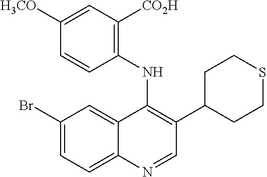 [SMILES: COc1ccc(Nc2c(C3CCSCC3)cnc3ccc(Br)cc23)c(C(=O)O)c1] |
| | 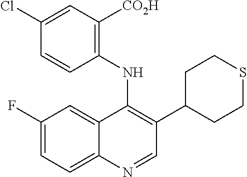 [SMILES: O=C(O)c1cc(Cl)ccc1Nc1c(C2CCSCC2)cnc2ccc(F)cc12] |
| | 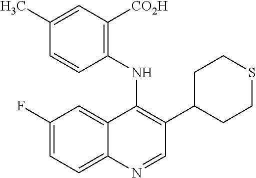 [SMILES: Cc1ccc(Nc2c(C3CCSCC3)cnc3ccc(F)cc23)c(C(=O)O)c1] |
| | 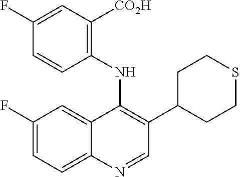 [SMILES: O=C(O)c1cc(F)ccc1Nc1c(C2CCSCC2)cnc2ccc(F)cc12] |
| | 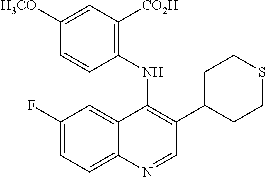 [SMILES: COc1ccc(Nc2c(C3CCSCC3)cnc3ccc(F)cc23)c(C(=O)O)c1] |
| | 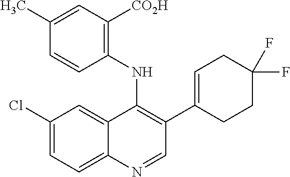 [SMILES: Cc1ccc(Nc2c(C3=CCC(F)(F)CC3)cnc3ccc(Cl)cc23)c(C(=O)O)c1] |
| | 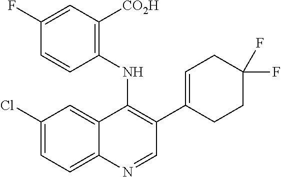 [SMILES: O=C(O)c1cc(F)ccc1Nc1c(C2=CCC(F)(F)CC2)cnc2ccc(Cl)cc12] |
| | 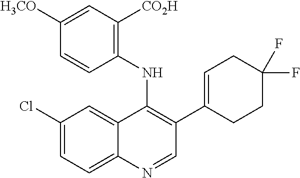 [SMILES: COc1ccc(Nc2c(C3=CCC(F)(F)CC3)cnc3ccc(Cl)cc23)c(C(=O)O)c1] |
| | 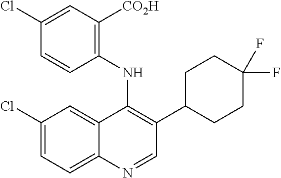 [SMILES: O=C(O)c1cc(Cl)ccc1Nc1c(C2CCC(F)(F)CC2)cnc2ccc(Cl)cc12] |
| | 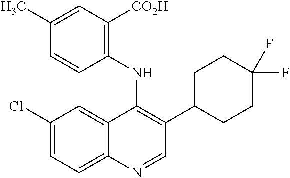 [SMILES: Cc1ccc(Nc2c(C3CCC(F)(F)CC3)cnc3ccc(Cl)cc23)c(C(=O)O)c1] |
| | 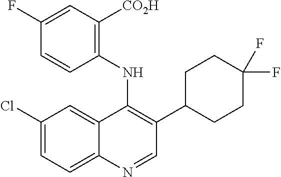 [SMILES: O=C(O)c1cc(F)ccc1Nc1c(C2CCC(F)(F)CC2)cnc2ccc(Cl)cc12] |
| | 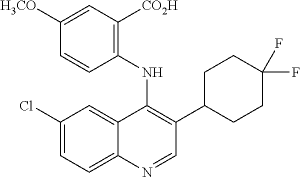 [SMILES: COc1ccc(Nc2c(C3CCC(F)(F)CC3)cnc3ccc(Cl)cc23)c(C(=O)O)c1] |
| | 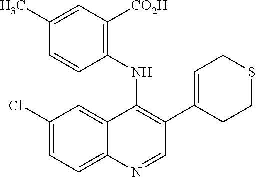 [SMILES: Cc1ccc(Nc2c(C3=CCSCC3)cnc3ccc(Cl)cc23)c(C(=O)O)c1] |
| | 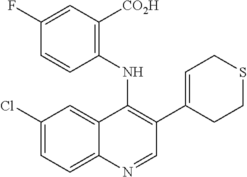 [SMILES: O=C(O)c1cc(F)ccc1Nc1c(C2=CCSCC2)cnc2ccc(Cl)cc12] |
| | 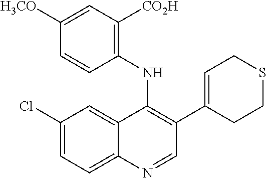 [SMILES: COc1ccc(Nc2c(C3=CCSCC3)cnc3ccc(Cl)cc23)c(C(=O)O)c1] |
| | 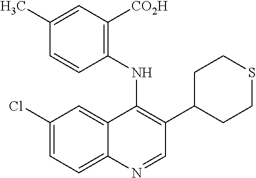 [SMILES: Cc1ccc(Nc2c(C3CCSCC3)cnc3ccc(Cl)cc23)c(C(=O)O)c1] |
| | 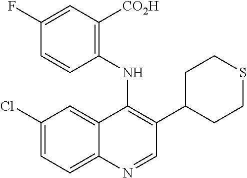 [SMILES: O=C(O)c1cc(F)ccc1Nc1c(C2CCSCC2)cnc2ccc(Cl)cc12] |
| | 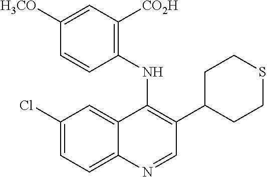 [SMILES: COc1ccc(Nc2c(C3CCSCC3)cnc3ccc(Cl)cc23)c(C(=O)O)c1] |
| BCH-396A | 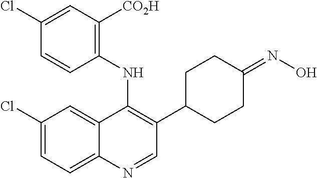 [SMILES: O=C(O)c1cc(Cl)ccc1Nc1c(C2CCC(=NO)CC2)cnc2ccc(Cl)cc12] |
| BCH-400A | 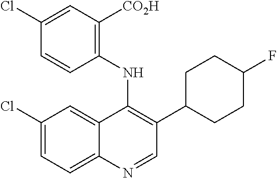 [SMILES: O=C(O)c1cc(Cl)ccc1Nc1c(C2CCC(F)CC2)cnc2ccc(Cl)cc12] |
| BCH-403A | 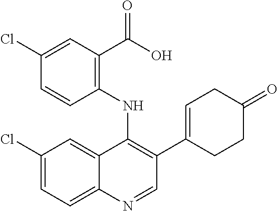 [SMILES: O=C1CC=C(c2cnc3ccc(Cl)cc3c2Nc2ccc(Cl)cc2C(=O)O)CC1] |
| BCH-404A | 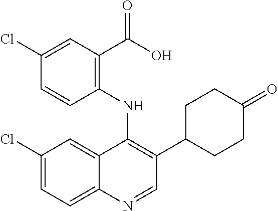 [SMILES: O=C1CCC(c2cnc3ccc(Cl)cc3c2Nc2ccc(Cl)cc2C(=O)O)CC1] |

or a pharmaceutically acceptable salt thereof.

In some embodiments, the present disclosure provides a compound of formula (IX):

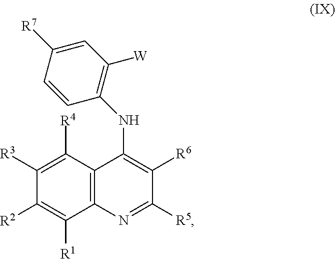

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is a 5-membered heterocycloalkyl;
R³ is halo; and
R⁷ is selected from halo, C₁₋₃ alkyl, and C₁₋₃ alkoxy.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo; In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (IX) has formula:

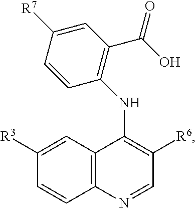

or a pharmaceutically acceptable salt thereof.

In some embodiments, R³ is selected from Cl, Br, and F. In some embodiments, R³ is Cl. In some embodiments, R³ is Br. In some embodiments, R³ is F.

In some embodiments, R⁶ is selected from tetrahydropyranyl and pyrrolidinyl.

In some embodiments, R⁶ is tetrahydropyranyl.

In some embodiments, R⁶ is pyrrolidinyl.

In some embodiments, R⁷ is halo.

In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl. In some embodiments, R⁷ is C₁₋₃ alkoxy. In some embodiments, R⁷ is methoxy. In some embodiments, R³ is F and R⁷ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (IX) is selected from any one of the following compounds:

TABLE IX

| | |
|---|---|
| BCH | 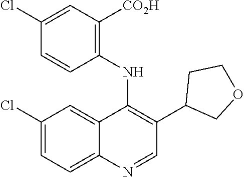 [SMILES: O=C(O)c1cc(Cl)ccc1Nc1c(C2CCOC2)cnc2ccc(Cl)cc12] |
| | 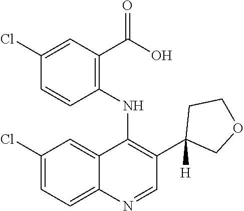 [SMILES: O=C(O)c1cc(Cl)ccc1Nc1c([C@@H]2CCOC2)cnc2ccc(Cl)cc12] |
| | 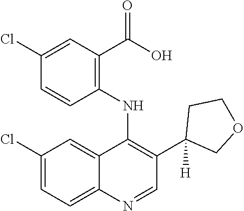 [SMILES: O=C(O)c1cc(Cl)ccc1Nc1c([C@H]2CCOC2)cnc2ccc(Cl)cc12] |
| BCH-355A | 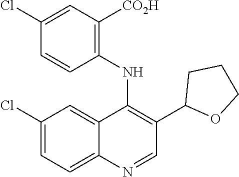 [SMILES: O=C(O)c1cc(Cl)ccc1Nc1c(C2CCCO2)cnc2ccc(Cl)cc12] |
| | 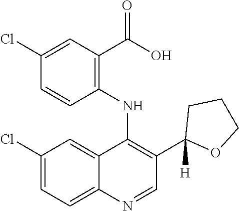 [SMILES: O=C(O)c1cc(Cl)ccc1Nc1c([C@@H]2CCCO2)cnc2ccc(Cl)cc12] |
| | 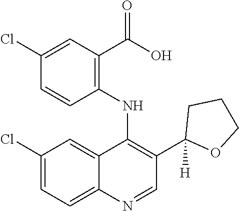 [SMILES: O=C(O)c1cc(Cl)ccc1Nc1c([C@H]2CCCO2)cnc2ccc(Cl)cc12] |
| BCH-401A | 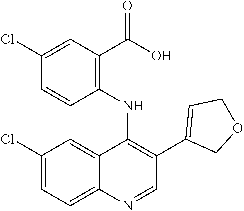 [SMILES: O=C(O)c1cc(Cl)ccc1Nc1c(C2=CCOC2)cnc2ccc(Cl)cc12] |
| BCH-357A | 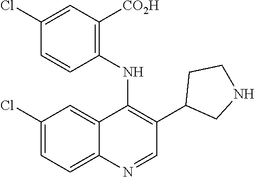 [SMILES: O=C(O)c1cc(Cl)ccc1Nc1c(C2CCNC2)cnc2ccc(Cl)cc12] |
| BCH-402A | 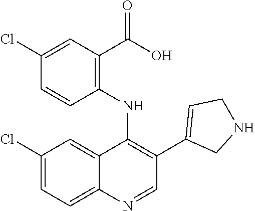 [SMILES: O=C(O)c1cc(Cl)ccc1Nc1c(C2=CCNC2)cnc2ccc(Cl)cc12] |
| | 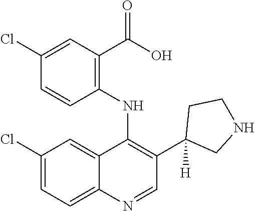 [SMILES: O=C(O)c1cc(Cl)ccc1Nc1c([C@H]2CCNC2)cnc2ccc(Cl)cc12] |
| | 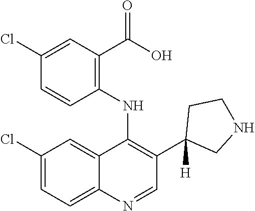 [SMILES: O=C(O)c1cc(Cl)ccc1Nc1c([C@@H]2CCNC2)cnc2ccc(Cl)cc12] |
| | 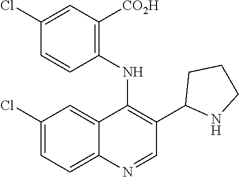 [SMILES: O=C(O)c1cc(Cl)ccc1Nc1c(C2CCCN2)cnc2ccc(Cl)cc12] |
| | 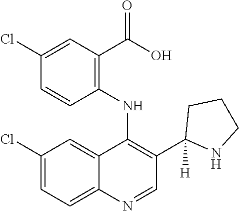 [SMILES: O=C(O)c1cc(Cl)ccc1Nc1c([C@H]2CCCN2)cnc2ccc(Cl)cc12] |
| | 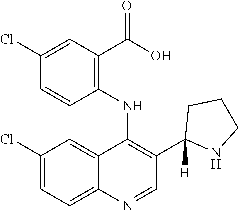 [SMILES: O=C(O)c1cc(Cl)ccc1Nc1c([C@@H]2CCCN2)cnc2ccc(Cl)cc12] |

or a pharmaceutically acceptable salt thereof.

In some embodiments, the present disclosure provides a compound of formula (X):

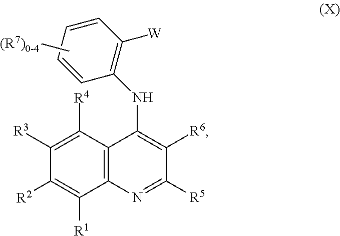

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is a 6-membered heteroaryl;
R³ is halo; and
R⁷ is selected from halo, C₁₋₃ alkyl, and C₁₋₃ alkoxy.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (X) has formula:

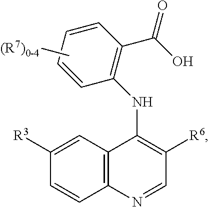

or a pharmaceutically acceptable salt thereof.

In some embodiments, R⁶ is selected from pyridinyl, triazinyl, and pyridazinyl.

In some embodiments, the compound of Formula (X) has formula:

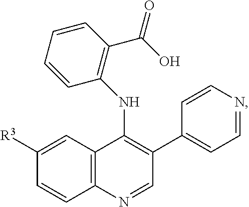

or a pharmaceutically acceptable salt thereof.

In some embodiments, the compound of Formula (X) has formula:

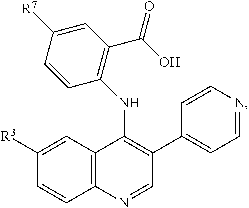

or a pharmaceutically acceptable salt thereof.

In some embodiments, the compound of Formula (X) has formula:

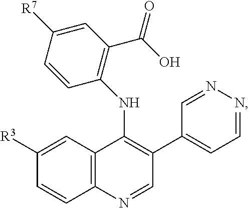

or a pharmaceutically acceptable salt thereof.

In some embodiments, the compound of Formula (X) has formula:

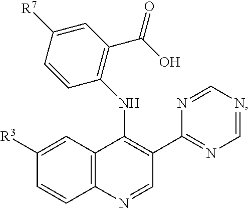

or a pharmaceutically acceptable salt thereof.

In some embodiments, R³ is selected from Cl, Br, and F. In some embodiments, R³ is Cl. In some embodiments, R³ is Br. In some embodiments, R³ is F.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R³ is F and R⁷ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (X) is selected from any one of the following compounds:

TABLE X

| | |
|---|---|
| BCH-209A | 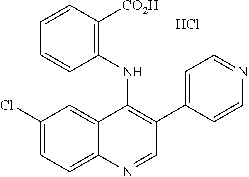 [SMILES: O=C(O)c1ccccc1Nc1c(-c2ccncc2)cnc2ccc(Cl)cc12 ; Cl] |
| | 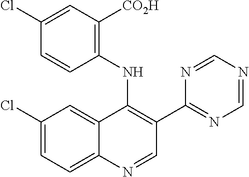 [SMILES: O=C(O)c1cc(Cl)ccc1Nc1c(-c2ncncn2)cnc2ccc(Cl)cc12] |
| | 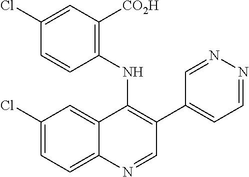 [SMILES: O=C(O)c1cc(Cl)ccc1Nc1c(-c2ccnnc2)cnc2ccc(Cl)cc12] |
| | 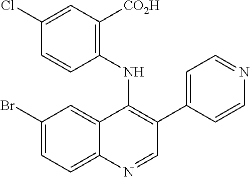 [SMILES: O=C(O)c1cc(Cl)ccc1Nc1c(-c2ccncc2)cnc2ccc(Br)cc12] |
| | 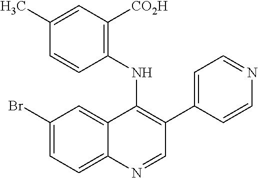 [SMILES: Cc1ccc(Nc2c(-c3ccncc3)cnc3ccc(Br)cc23)c(C(=O)O)c1] |
| | 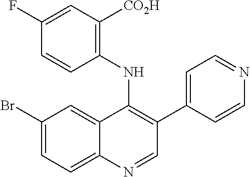 [SMILES: O=C(O)c1cc(F)ccc1Nc1c(-c2ccncc2)cnc2ccc(Br)cc12] |
| | 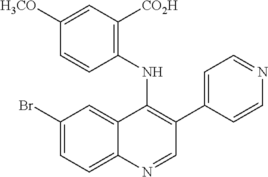 [SMILES: COc1ccc(Nc2c(-c3ccncc3)cnc3ccc(Br)cc23)c(C(=O)O)c1] |
| | 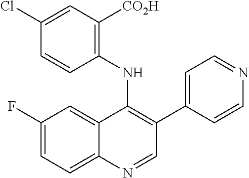 [SMILES: O=C(O)c1cc(Cl)ccc1Nc1c(-c2ccncc2)cnc2ccc(F)cc12] |
| | 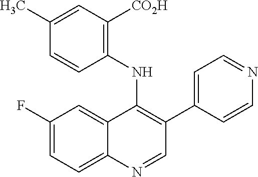 [SMILES: Cc1ccc(Nc2c(-c3ccncc3)cnc3ccc(F)cc23)c(C(=O)O)c1] |
| | 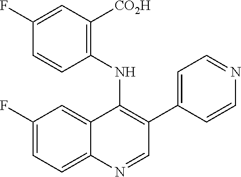 [SMILES: O=C(O)c1cc(F)ccc1Nc1c(-c2ccncc2)cnc2ccc(F)cc12] |
| | 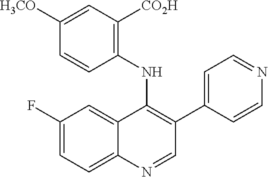 [SMILES: COc1ccc(Nc2c(-c3ccncc3)cnc3ccc(F)cc23)c(C(=O)O)c1] |
| BCH-391A | 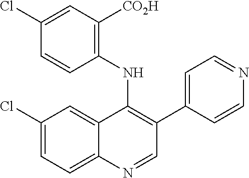 [SMILES: O=C(O)c1cc(Cl)ccc1Nc1c(-c2ccncc2)cnc2ccc(Cl)cc12] |
| | 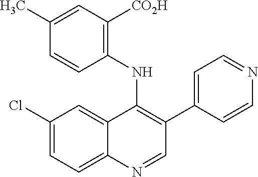 [SMILES: Cc1ccc(Nc2c(-c3ccncc3)cnc3ccc(Cl)cc23)c(C(=O)O)c1] |
| | 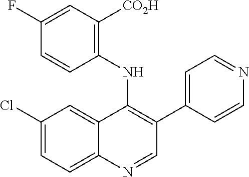 [SMILES: O=C(O)c1cc(F)ccc1Nc1c(-c2ccncc2)cnc2ccc(Cl)cc12] |
| | 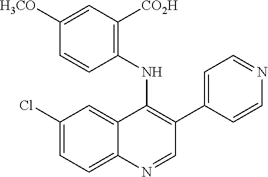 [SMILES: COc1ccc(Nc2c(-c3ccncc3)cnc3ccc(Cl)cc23)c(C(=O)O)c1] |

or a pharmaceutically acceptable salt thereof.

In some embodiments, the present disclosure provides a compound of formula (XT):

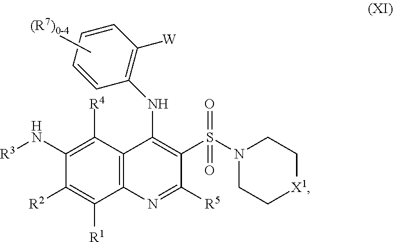

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is selected from 5-6 membered heterocycloalkyl, C₄₋₆ cycloalkyl, and 5-9 membered heteroaryl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl; and
R⁷ is selected from halo, C₁₋₃ alkyl, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo; In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (XI) has formula:

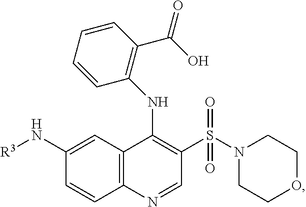

or a pharmaceutically acceptable salt thereof.

In some embodiments, R³ is selected from tetrahydropyranyl, cyclohexyl, piperidinyl, 1,1-dioxo tetrahydro-2H-thiopyranyl, pyridinyl, pyrimidinyl, oxazolyl, thioxazolyl, thiazolyl, and benzimidazolyl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl.

In some embodiments, R³ is selected from tetrahydropyranyl, cyclohexyl, piperidinyl, pyridinyl, oxazolyl, and benzimidazolyl, each of which is optionally substituted with halo.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (XI) is selected from any one of the following compounds:

TABLE XI

| | |
|---|---|
| BCH- 237A | 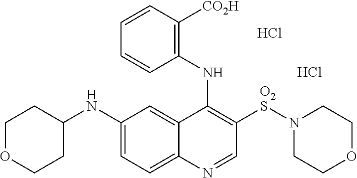 [SMILES: O=C(O)c1ccccc1Nc1c(S(=O)(=O)N2CCOCC2)cnc2ccc(NC3CCOCC3)cc12 ; Cl ; Cl] |
| BCH- 238A | 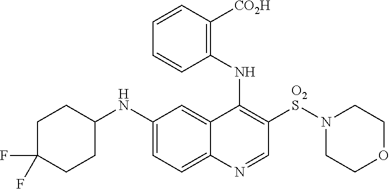 [SMILES: O=C(O)c1ccccc1Nc1c(S(=O)(=O)N2CCOCC2)cnc2ccc(NC3CCC(F)(F)CC3)cc12] |
| BCH- 239A | 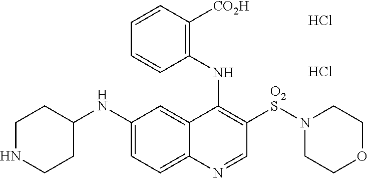 [SMILES: O=C(O)c1ccccc1Nc1c(S(=O)(=O)N2CCOCC2)cnc2ccc(NC3CCNCC3)cc12 ; Cl ; Cl] |
| BCH- 240A | 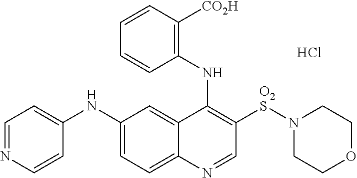 [SMILES: O=C(O)c1ccccc1Nc1c(S(=O)(=O)N2CCOCC2)cnc2ccc(Nc3ccncc3)cc12 ; Cl] |
| BCH- 241A | 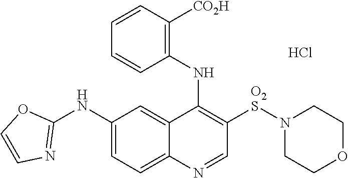 [SMILES: O=C(O)c1ccccc1Nc1c(S(=O)(=O)N2CCOCC2)cnc2ccc(Nc3ncco3)cc12 ; Cl] |
| BCH- 247A | 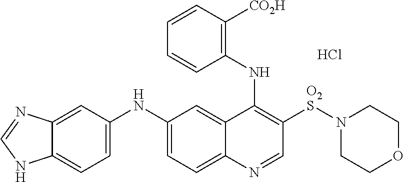 [SMILES: O=C(O)c1ccccc1Nc1c(S(=O)(=O)N2CCOCC2)cnc2ccc(Nc3ccc4[nH]cnc4c3)cc12 ; Cl] |

or a pharmaceutically acceptable salt thereof.

In some embodiments, the present disclosure provides a compound of formula (XII):

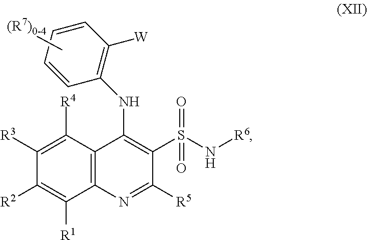

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is selected from 5-6 membered heterocycloalkyl, C₄₋₆ cycloalkyl, C₆₋₁₀ aryl, and a 5-6 membered heteroaryl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl;
R³ is halo; and
each R⁷ is independently selected from halo, C₁₋₃ alkyl, C₁₋₃ haloalkyl, C₁₋₃ haloalkoxy, and C₁₋₃ alkoxy.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (XII) has formula:

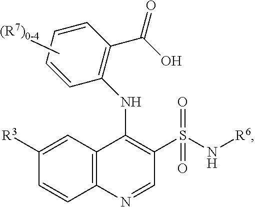

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁶ is selected from tetrahydropyranyl, cyclohexyl, piperidinyl, pyridinyl, phenyl, oxadiazolyl, tetrazolyl, pyrimidinyl, oxazolyl, thioxazolyl, and thiazolyl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl.

In some embodiments, R⁶ is selected from tetrahydropyranyl, cyclohexyl, piperidinyl, pyridinyl, phenyl, oxadiazolyl, and tetrazolyl, each of which is optionally substituted with halo or C₁₋₃ alkyl.

In some embodiments, R⁷ is selected from halo, C₁₋₃ alkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (XII) is selected from any one of the following compounds:

TABLE XII

| | |
|---|---|
| BCH- 216A | 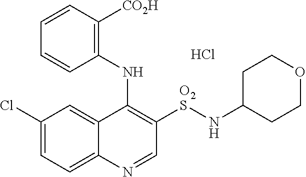 [SMILES: O=C(O)c1ccccc1Nc1c(S(=O)(=O)NC2CCOCC2)cnc2ccc(Cl)cc12 ; Cl] |
| BCH- 217A | 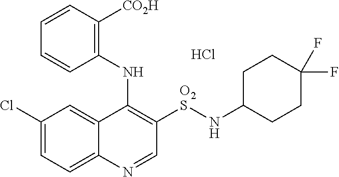 [SMILES: O=C(O)c1ccccc1Nc1c(S(=O)(=O)NC2CCC(F)(F)CC2)cnc2ccc(Cl)cc12 ; Cl] |
| BCH- 219A | 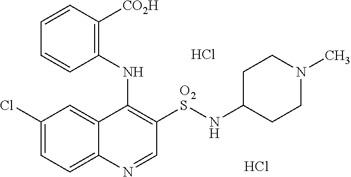 [SMILES: CN1CCC(NS(=O)(=O)c2cnc3ccc(Cl)cc3c2Nc2ccccc2C(=O)O)CC1 ; Cl ; Cl] |
| BCH- 221A | 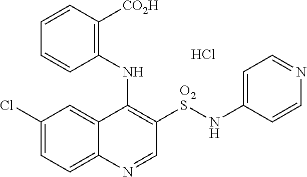 [SMILES: O=C(O)c1ccccc1Nc1c(S(=O)(=O)Nc2ccncc2)cnc2ccc(Cl)cc12 ; Cl] |
| BCH- 226A | 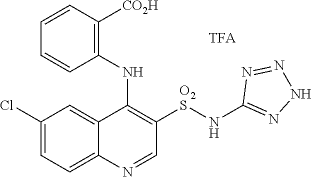 [SMILES: O=C(O)c1ccccc1Nc1c(S(=O)(=O)Nc2nn[nH]n2)cnc2ccc(Cl)cc12 ; "TFA"] |
| BCH- 231A | 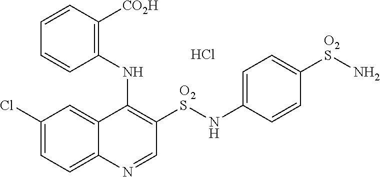 [SMILES: NS(=O)(=O)c1ccc(NS(=O)(=O)c2cnc3ccc(Cl)cc3c2Nc2ccccc2C(=O)O)cc1 ; Cl] |
| 225A | 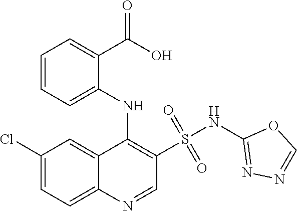 [SMILES: O=C(O)c1ccccc1Nc1c(S(=O)(=O)Nc2nnco2)cnc2ccc(Cl)cc12] |
| | 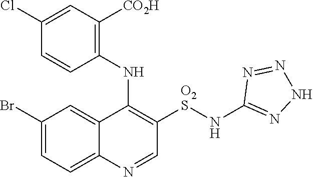 [SMILES: O=C(O)c1cc(Cl)ccc1Nc1c(S(=O)(=O)Nc2nn[nH]n2)cnc2ccc(Br)cc12] |
| | 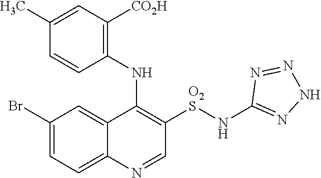 [SMILES: Cc1ccc(Nc2c(S(=O)(=O)Nc3nn[nH]n3)cnc3ccc(Br)cc23)c(C(=O)O)c1] |
| | 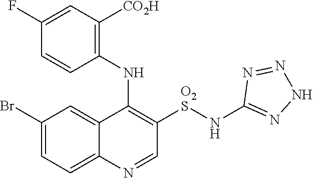 [SMILES: O=C(O)c1cc(F)ccc1Nc1c(S(=O)(=O)Nc2nn[nH]n2)cnc2ccc(Br)cc12] |
| | 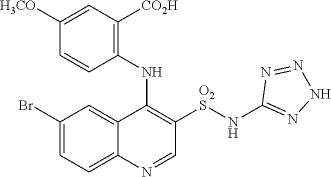 [SMILES: COc1ccc(Nc2c(S(=O)(=O)Nc3nn[nH]n3)cnc3ccc(Br)cc23)c(C(=O)O)c1] |
| | 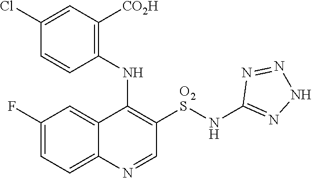 [SMILES: O=C(O)c1cc(Cl)ccc1Nc1c(S(=O)(=O)Nc2nn[nH]n2)cnc2ccc(F)cc12] |
| | 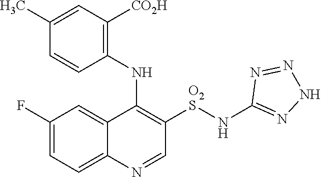 [SMILES: Cc1ccc(Nc2c(S(=O)(=O)Nc3nn[nH]n3)cnc3ccc(F)cc23)c(C(=O)O)c1] |
| | 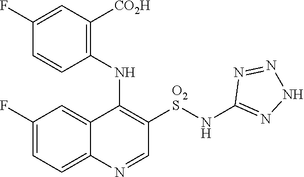 [SMILES: O=C(O)c1cc(F)ccc1Nc1c(S(=O)(=O)Nc2nn[nH]n2)cnc2ccc(F)cc12] |
| | 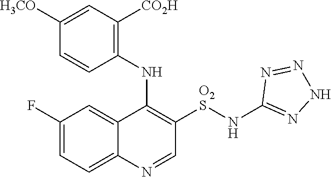 [SMILES: COc1ccc(Nc2c(S(=O)(=O)Nc3nn[nH]n3)cnc3ccc(F)cc23)c(C(=O)O)c1] |
| | 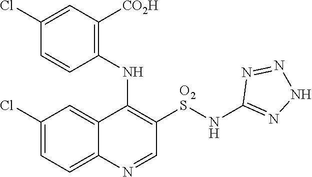 [SMILES: O=C(O)c1cc(Cl)ccc1Nc1c(S(=O)(=O)Nc2nn[nH]n2)cnc2ccc(Cl)cc12] |
| | 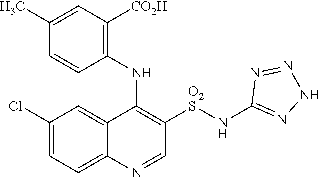 [SMILES: Cc1ccc(Nc2c(S(=O)(=O)Nc3nn[nH]n3)cnc3ccc(Cl)cc23)c(C(=O)O)c1] |
| | 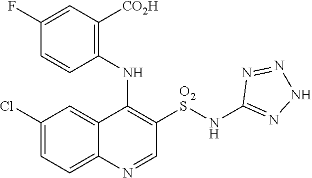 [SMILES: O=C(O)c1cc(F)ccc1Nc1c(S(=O)(=O)Nc2nn[nH]n2)cnc2ccc(Cl)cc12] |
| | 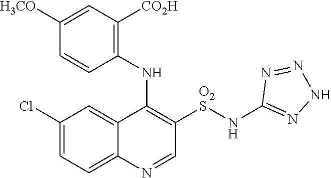 [SMILES: COc1ccc(Nc2c(S(=O)(=O)Nc3nn[nH]n3)cnc3ccc(Cl)cc23)c(C(=O)O)c1] |

or a pharmaceutically acceptable salt thereof.

In some embodiments, the present disclosure provides a compound of formula (XIII):

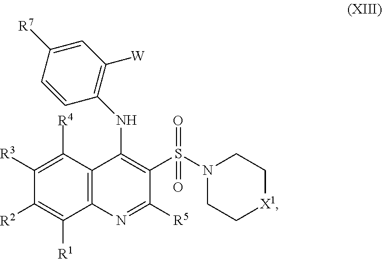

or a pharmaceutically acceptable salt thereof, wherein:
X¹ is selected from O, S, and SO₂;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo;
R⁷ is selected from C═O(OH), halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy; and
R^{7′} and R^{7″} are each independently selected from H, halo, CN, C₁₋₃ alkyl, and C₁₋₃ haloalkyl.

In some embodiments:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

In some embodiments, the compound has formula:

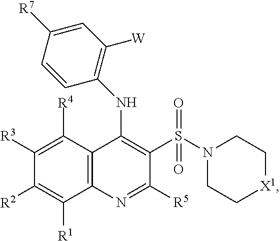

or a pharmaceutically acceptable salt thereof, wherein:
X¹ is selected from O and S; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

In some embodiments, X¹ is selected from O and S.

In some embodiments, X¹ is O.

In some embodiments, X¹ is S.

In some embodiments, X¹ is SO₂.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (XIII) has formula:

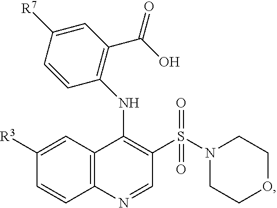

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R⁷ is B(OH)₂. In some embodiments, R⁷ is OH. In some embodiments, R⁷ is CN. In some embodiments, R⁷ is C₁₋₃ haloalkyl. In some embodiments, R⁷ is HO—C₁₋₃ haloalkyl. In some embodiments, R⁷ is aminosulfonyl. In some embodiments, R⁷ is C₁₋₃ haloalkylcarbonyl. In some embodiments, R⁷ is C₁₋₃ alkylcarbonyl. In some embodiments, R⁷ is carbamyl.

In some embodiments, R^{7′} is selected from H, halo, CN, C₁₋₃ alkyl, and C₁₋₃ haloalkyl. In some embodiments, R^{7″} is selected from H, halo, CN, C₁₋₃ alkyl, and C₁₋₃ haloalkyl.

In some embodiments, R^{7′} and R^{7″} are each independently selected from H, halo, and C₁₋₃ alkyl. In some embodiments, R^{7′} is H. In some embodiments, R^{7′} is halo. In some embodiments, R^{7′} is C₁₋₃ alkyl. In some embodiments, R^{7″} is H. In some embodiments, R^{7″} is halo. In some embodiments, R^{7″} is C₁₋₃ alkyl.

In some embodiments, R³ is F and R⁵ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (XIII) is selected from any one of the following compounds:

TABLE XIII

| | |
|---|---|
| BCH-263A | 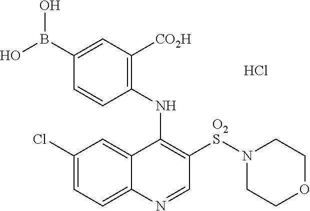 [SMILES: O=C(O)c1cc(B(O)O)ccc1Nc1c(S(=O)(=O)N2CCOCC2)cnc2ccc(Cl)cc12 ; Cl] |
| BCH-265A | 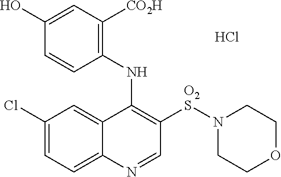 [SMILES: O=C(O)c1cc(O)ccc1Nc1c(S(=O)(=O)N2CCOCC2)cnc2ccc(Cl)cc12 ; Cl] |
| BCH-266A | 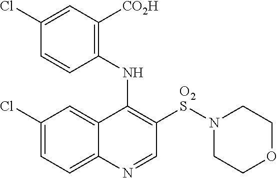 [SMILES: O=C(O)c1cc(Cl)ccc1Nc1c(S(=O)(=O)N2CCOCC2)cnc2ccc(Cl)cc12] |
| BCH-267A | 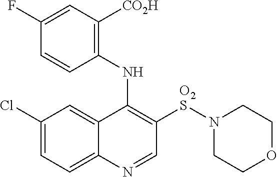 [SMILES: O=C(O)c1cc(F)ccc1Nc1c(S(=O)(=O)N2CCOCC2)cnc2ccc(Cl)cc12] |
| BCH-268A | 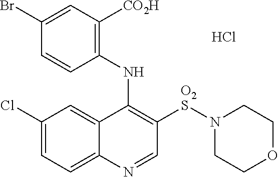 [SMILES: O=C(O)c1cc(Br)ccc1Nc1c(S(=O)(=O)N2CCOCC2)cnc2ccc(Cl)cc12 ; Cl] |
| BCH-269A | 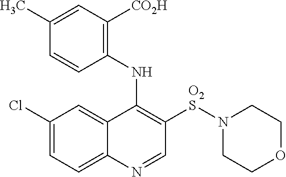 [SMILES: Cc1ccc(Nc2c(S(=O)(=O)N3CCOCC3)cnc3ccc(Cl)cc23)c(C(=O)O)c1] |
| BCH-270A | 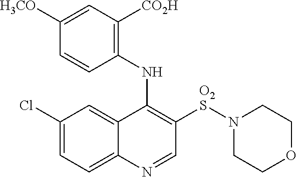 [SMILES: COc1ccc(Nc2c(S(=O)(=O)N3CCOCC3)cnc3ccc(Cl)cc23)c(C(=O)O)c1] |
| BCH-271A | 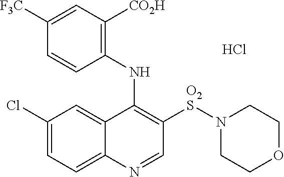 [SMILES: O=C(O)c1cc(C(F)(F)F)ccc1Nc1c(S(=O)(=O)N2CCOCC2)cnc2ccc(Cl)cc12 ; Cl] |
| BCH-290A | 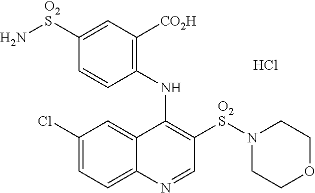 [SMILES: NS(=O)(=O)c1ccc(Nc2c(S(=O)(=O)N3CCOCC3)cnc3ccc(Cl)cc23)c(C(=O)O)c1 ; Cl] |
| BCH-293A | 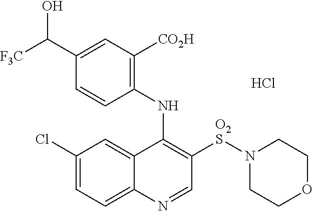 [SMILES: O=C(O)c1cc(C(O)C(F)(F)F)ccc1Nc1c(S(=O)(=O)N2CCOCC2)cnc2ccc(Cl)cc12 ; Cl] |
| BCH-262A | 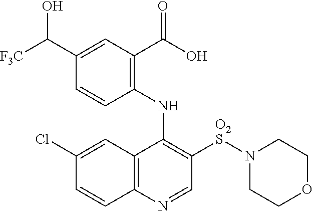 [SMILES: O=C(O)c1cc(C(O)C(F)(F)F)ccc1Nc1c(S(=O)(=O)N2CCOCC2)cnc2ccc(Cl)cc12] |
| BCH-318A | 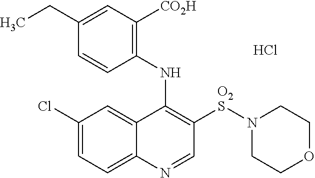 [SMILES: CCc1ccc(Nc2c(S(=O)(=O)N3CCOCC3)cnc3ccc(Cl)cc23)c(C(=O)O)c1 ; Cl] |
| BCH-319A | 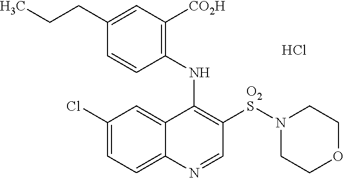 [SMILES: CCCc1ccc(Nc2c(S(=O)(=O)N3CCOCC3)cnc3ccc(Cl)cc23)c(C(=O)O)c1 ; Cl] |
| BCH_320A | 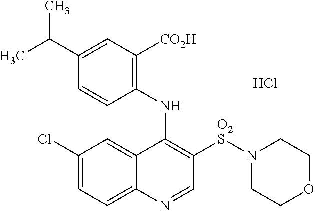 [SMILES: CC(C)c1ccc(Nc2c(S(=O)(=O)N3CCOCC3)cnc3ccc(Cl)cc23)c(C(=O)O)c1 ; Cl] |
| BCH-338A | 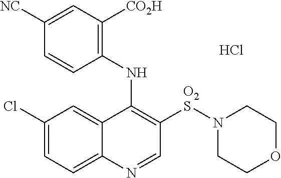 [SMILES: N#Cc1ccc(Nc2c(S(=O)(=O)N3CCOCC3)cnc3ccc(Cl)cc23)c(C(=O)O)c1 ; Cl] |
| 349A | 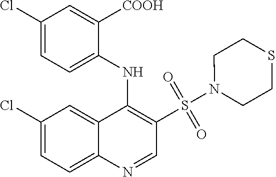 [SMILES: O=C(O)c1cc(Cl)ccc1Nc1c(S(=O)(=O)N2CCSCC2)cnc2ccc(Cl)cc12] |
| | 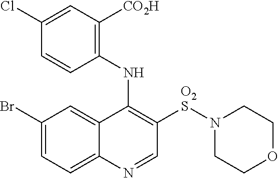 [SMILES: O=C(O)c1cc(Cl)ccc1Nc1c(S(=O)(=O)N2CCOCC2)cnc2ccc(Br)cc12] |
| | 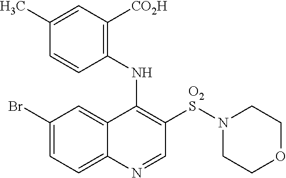 [SMILES: Cc1ccc(Nc2c(S(=O)(=O)N3CCOCC3)cnc3ccc(Br)cc23)c(C(=O)O)c1] |
| | 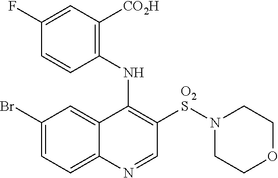 [SMILES: O=C(O)c1cc(F)ccc1Nc1c(S(=O)(=O)N2CCOCC2)cnc2ccc(Br)cc12] |
| | 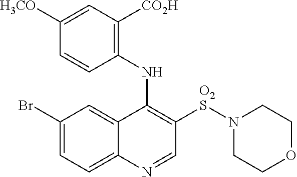 [SMILES: COc1ccc(Nc2c(S(=O)(=O)N3CCOCC3)cnc3ccc(Br)cc23)c(C(=O)O)c1] |
| | 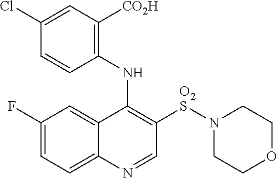 [SMILES: O=C(O)c1cc(Cl)ccc1Nc1c(S(=O)(=O)N2CCOCC2)cnc2ccc(F)cc12] |
| | 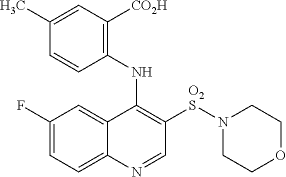 [SMILES: Cc1ccc(Nc2c(S(=O)(=O)N3CCOCC3)cnc3ccc(F)cc23)c(C(=O)O)c1] |
| | 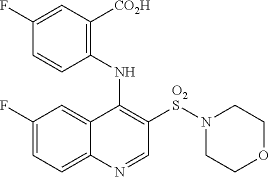 [SMILES: O=C(O)c1cc(F)ccc1Nc1c(S(=O)(=O)N2CCOCC2)cnc2ccc(F)cc12] |
| | 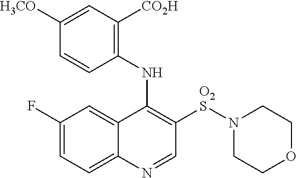 [SMILES: COc1ccc(Nc2c(S(=O)(=O)N3CCOCC3)cnc3ccc(F)cc23)c(C(=O)O)c1] |
| | 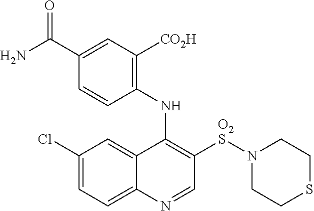 [SMILES: NC(=O)c1ccc(Nc2c(S(=O)(=O)N3CCSCC3)cnc3ccc(Cl)cc23)c(C(=O)O)c1] |
| BCH-373A | 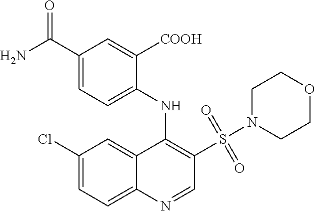 [SMILES: NC(=O)c1ccc(Nc2c(S(=O)(=O)N3CCOCC3)cnc3ccc(Cl)cc23)c(C(=O)O)c1] |
| BCH-411A | 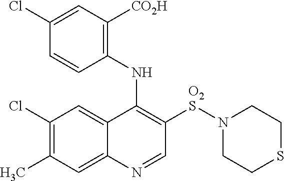 [SMILES: Cc1cc2ncc(S(=O)(=O)N3CCSCC3)c(Nc3ccc(Cl)cc3C(=O)O)c2cc1Cl] |
| BCH-413A | 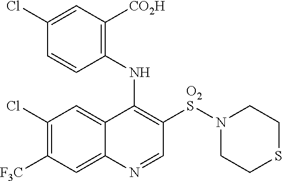 [SMILES: O=C(O)c1cc(Cl)ccc1Nc1c(S(=O)(=O)N2CCSCC2)cnc2cc(C(F)(F)F)c(Cl)cc12] |
| BCH-414A | 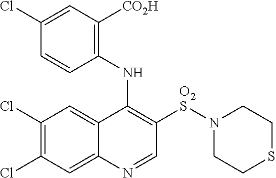 [SMILES: O=C(O)c1cc(Cl)ccc1Nc1c(S(=O)(=O)N2CCSCC2)cnc2cc(Cl)c(Cl)cc12] |
| BCH-415A | 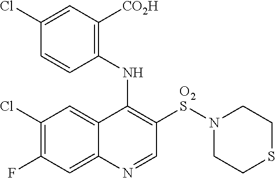 [SMILES: O=C(O)c1cc(Cl)ccc1Nc1c(S(=O)(=O)N2CCSCC2)cnc2cc(F)c(Cl)cc12] |
| BCH-416A | 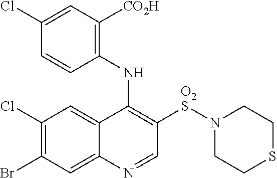 [SMILES: O=C(O)c1cc(Cl)ccc1Nc1c(S(=O)(=O)N2CCSCC2)cnc2cc(Br)c(Cl)cc12] |
| BCH-417A | 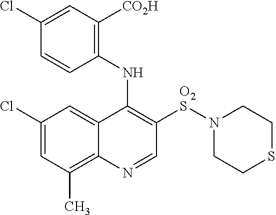 [SMILES: Cc1cc(Cl)cc2c(Nc3ccc(Cl)cc3C(=O)O)c(S(=O)(=O)N3CCSCC3)cnc12] |
| BCH-418A | 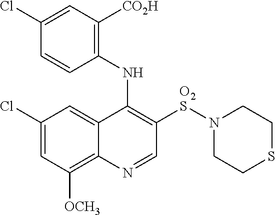 [SMILES: COc1cc(Cl)cc2c(Nc3ccc(Cl)cc3C(=O)O)c(S(=O)(=O)N3CCSCC3)cnc12] |
| BCH-419A | 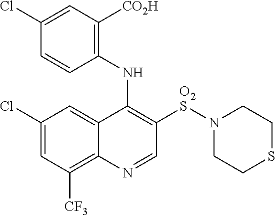 [SMILES: O=C(O)c1cc(Cl)ccc1Nc1c(S(=O)(=O)N2CCSCC2)cnc2c(C(F)(F)F)cc(Cl)cc12] |
| BCH-420A | 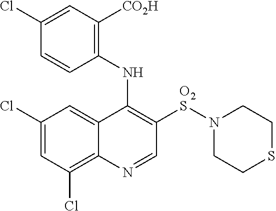 [SMILES: O=C(O)c1cc(Cl)ccc1Nc1c(S(=O)(=O)N2CCSCC2)cnc2c(Cl)cc(Cl)cc12] |
| BCH-421A | 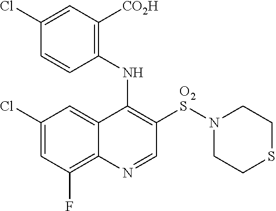 [SMILES: O=C(O)c1cc(Cl)ccc1Nc1c(S(=O)(=O)N2CCSCC2)cnc2c(F)cc(Cl)cc12] |
| BCH-422A | 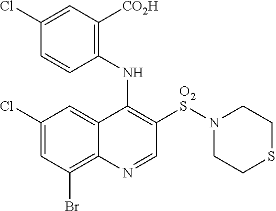 [SMILES: O=C(O)c1cc(Cl)ccc1Nc1c(S(=O)(=O)N2CCSCC2)cnc2c(Br)cc(Cl)cc12] |
| BCH-423A | 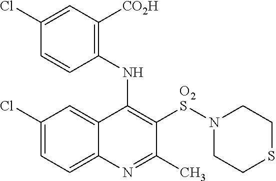 [SMILES: Cc1nc2ccc(Cl)cc2c(Nc2ccc(Cl)cc2C(=O)O)c1S(=O)(=O)N1CCSCC1] |
| BCH-398A | 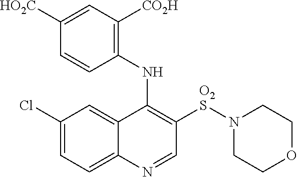 [SMILES: O=C(O)c1ccc(Nc2c(S(=O)(=O)N3CCOCC3)cnc3ccc(Cl)cc23)c(C(=O)O)c1] |
| BCH-425A | 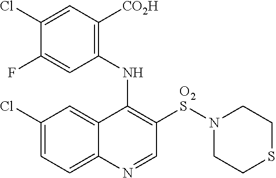 [SMILES: O=C(O)c1cc(Cl)c(F)cc1Nc1c(S(=O)(=O)N2CCSCC2)cnc2ccc(Cl)cc12] |
| BCH-426A | 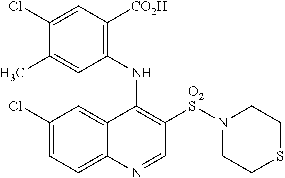 [SMILES: Cc1cc(Nc2c(S(=O)(=O)N3CCSCC3)cnc3ccc(Cl)cc23)c(C(=O)O)cc1Cl] |
| BCH-427A | 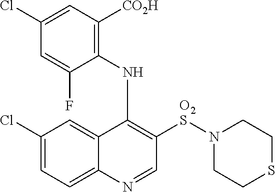 [SMILES: O=C(O)c1cc(Cl)cc(F)c1Nc1c(S(=O)(=O)N2CCSCC2)cnc2ccc(Cl)cc12] |
| BCH-429A | 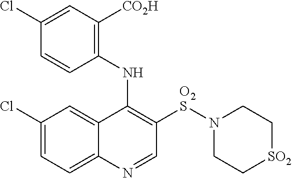 [SMILES: O=C(O)c1cc(Cl)ccc1Nc1c(S(=O)(=O)N2CCS(=O)(=O)CC2)cnc2ccc(Cl)cc12] |

or a pharmaceutically acceptable salt thereof.

In some embodiments, the present disclosure provides a compound of formula

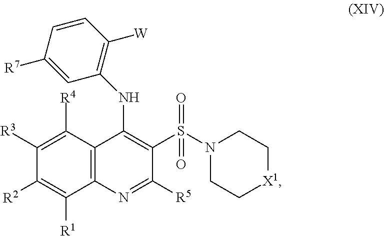

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo:

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (XIV) has formula:

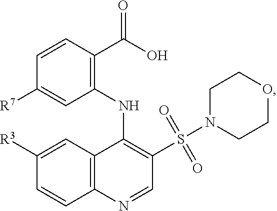

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁷ is selected from halo, B(OH)₂, OH, C₁₋₃ alkyl, C₁₋₃ haloalkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R⁷ is B(OH)₂. In some embodiments. R⁷ is OH. In some embodiments, R⁷ is CN. In some embodiments, R⁷ is C₁₋₃ haloalkyl. In some embodiments, R⁷ is HO—C₁₋₃ haloalkyl. In some embodiments, R⁷ is aminosulfonyl. In some embodiments, R⁷ is C₁₋₃ haloalkylcarbonyl. In some embodiments, R⁷ is C₁₋₃ alkylcarbonyl. In some embodiments, R⁷ is carbamyl.

In some embodiments, R³ is F and R⁵ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (XIV) is selected from any one of the following compounds:

TABLE XIV

| | |
|---|---|
| BCH-272A | 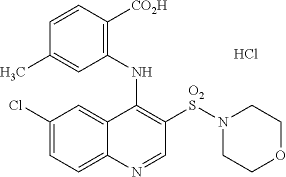 [SMILES: Cc1ccc(C(=O)O)c(Nc2c(S(=O)(=O)N3CCOCC3)cnc3ccc(Cl)cc23)c1 ; Cl] |
| BCH-273A | 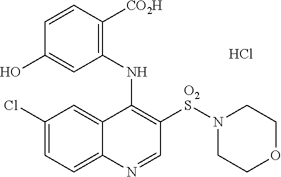 [SMILES: O=C(O)c1ccc(O)cc1Nc1c(S(=O)(=O)N2CCOCC2)cnc2ccc(Cl)cc12 ; Cl] |
| BCH-274A | 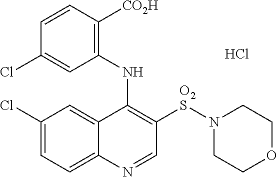 [SMILES: O=C(O)c1ccc(Cl)cc1Nc1c(S(=O)(=O)N2CCOCC2)cnc2ccc(Cl)cc12 ; Cl] |
| BCH-275A | 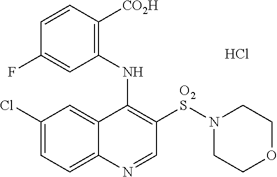 [SMILES: O=C(O)c1ccc(F)cc1Nc1c(S(=O)(=O)N2CCOCC2)cnc2ccc(Cl)cc12 ; Cl] |
| BCH-276A | 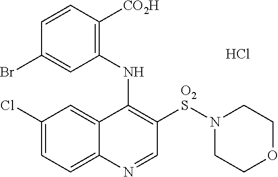 [SMILES: O=C(O)c1ccc(Br)cc1Nc1c(S(=O)(=O)N2CCOCC2)cnc2ccc(Cl)cc12 ; Cl] |
| BCH-277A | 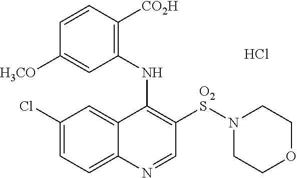 [SMILES: COc1ccc(C(=O)O)c(Nc2c(S(=O)(=O)N3CCOCC3)cnc3ccc(Cl)cc23)c1 ; Cl] |
| BCH-278A | 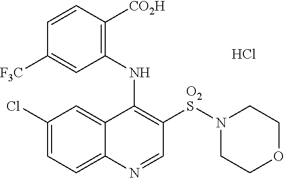 [SMILES: O=C(O)c1ccc(C(F)(F)F)cc1Nc1c(S(=O)(=O)N2CCOCC2)cnc2ccc(Cl)cc12 ; Cl] |
| BCH-289A | 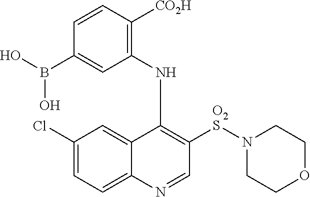 [SMILES: O=C(O)c1ccc(B(O)O)cc1Nc1c(S(=O)(=O)N2CCOCC2)cnc2ccc(Cl)cc12] |

or a pharmaceutically acceptable salt thereof.

In some embodiments, the present disclosure provides a compound of formula (XV):

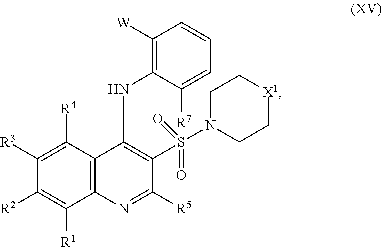

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

In some embodiments, X¹ is O.

In some embodiments, X¹ is S.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (XV) has formula:

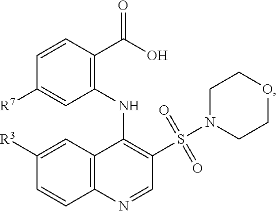

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁷ is selected from halo, B(OH)₂, OH, C₁₋₃ alkyl, C₁₋₃ haloalkyl, and C₁₋₃ alkoxy.

In some embodiments, R⁷ is selected from halo, OH, C₁₋₃ alkyl, C₁₋₃ haloalkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R⁷ is B(OH)₂. In some embodiments, R⁷ is OH. In some embodiments, R⁷ is CN. In some embodiments, R⁷ is C₁₋₃ haloalkyl. In some embodiments, R⁷ is HO—C₁₋₃ haloalkyl. In some embodiments, R⁷ is aminosulfonyl. In some embodiments, R⁷ is C₁₋₃ haloalkylcarbonyl. In some embodiments, R⁷ is C₁₋₃ alkylcarbonyl. In some embodiments, R⁷ is carbamyl.

In some embodiments, R³ is F and R⁷ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (XV) is selected from any one of the following compounds:

TABLE XV

| | |
|---|---|
| BCH-279A | 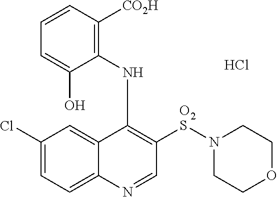 [SMILES: O=C(O)c1cccc(O)c1Nc1c(S(=O)(=O)N2CCOCC2)cnc2ccc(Cl)cc12 ; Cl] |
| BCH-280A | 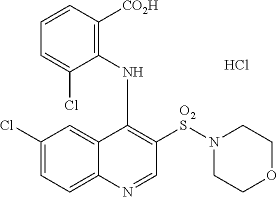 [SMILES: O=C(O)c1cccc(Cl)c1Nc1c(S(=O)(=O)N2CCOCC2)cnc2ccc(Cl)cc12 ; Cl] |
| BCH-281A | 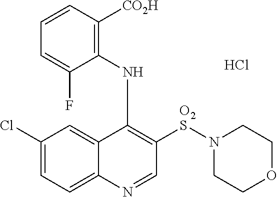 [SMILES: O=C(O)c1cccc(F)c1Nc1c(S(=O)(=O)N2CCOCC2)cnc2ccc(Cl)cc12 ; Cl] |
| BCH-282A | 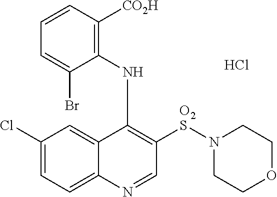 [SMILES: O=C(O)c1cccc(Br)c1Nc1c(S(=O)(=O)N2CCOCC2)cnc2ccc(Cl)cc12 ; Cl] |
| BCH-283A | 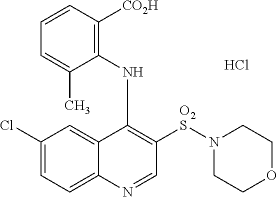 [SMILES: Cc1cccc(C(=O)O)c1Nc1c(S(=O)(=O)N2CCOCC2)cnc2ccc(Cl)cc12 ; Cl] |
| BCH-284A | 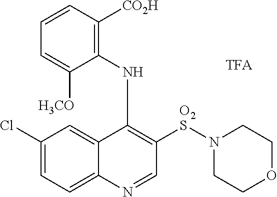 [SMILES: COc1cccc(C(=O)O)c1Nc1c(S(=O)(=O)N2CCOCC2)cnc2ccc(Cl)cc12 ; "TFA"] |
| BCH-285A | 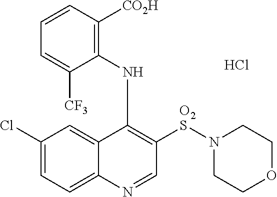 [SMILES: O=C(O)c1cccc(C(F)(F)F)c1Nc1c(S(=O)(=O)N2CCOCC2)cnc2ccc(Cl)cc12 ; Cl] |

or a pharmaceutically acceptable salt thereof.

In some embodiments, the present disclosure provides a compound of formula (XVI):

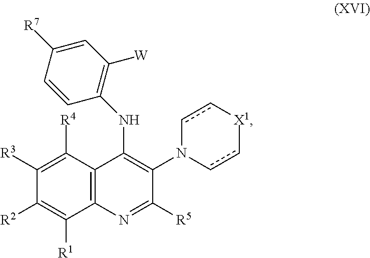

or a pharmaceutically acceptable salt thereof, wherein:
X¹ is selected from O, S, CF₂, C═O, C═N—OH, CHOH, CHCl, CHF, CH(OCF₃), CCl₂, NH, NCH₃, Si(OH)₂, SO₂, and cyclopropylidene;
each [symbol] is independently a single bond or a double bond;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

In some embodiments, X¹ is selected from O, S, CF₂, C═N—OH, CHCl, CCl₂, NH, NCH₃, Si(OH)₂, SO₂, and cyclopropylidene.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

X¹ is selected from O, S, CF₂, C═N—OH, NH, NCH₃, and SO₂.

In some embodiments, X¹ is O. In some embodiments, X¹ is S. In some embodiments, X¹ is CF₂. In some embodiments, X¹ is CHCl. In some embodiments, X¹ is CCl₂. In some embodiments, X¹ is NH. In some embodiments, X¹ is NCH₃. In some embodiments, X¹ is Si(OH)₂. In some embodiments, X¹ is SO₂. In some embodiments, X¹ is cyclopropylidene. In some embodiments, X¹ is C═N—OH. In some embodiments, X¹ is C═O. In some embodiments, X¹ is CHOH. In some embodiments, X¹ is CHF. In some embodiments, X¹ is CH(OCF₃).

In some embodiments, the compound of Formula (XVI) has formula:

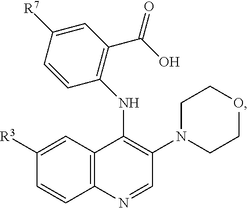

or a pharmaceutically acceptable salt thereof.

In some embodiments, the compound of Formula (XVI) has formula:

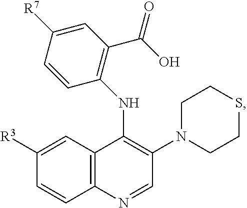

or a pharmaceutically acceptable salt thereof.

In some embodiments, the compound of Formula (XVI) has formula:

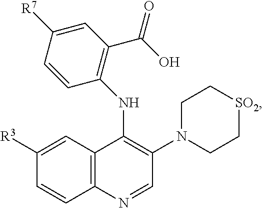

or a pharmaceutically acceptable salt thereof.

In some embodiments, the compound of Formula (XVI) has formula:

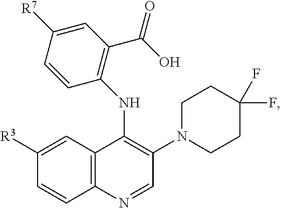

or a pharmaceutically acceptable salt thereof.

In some embodiments, the compound of Formula (XVI) has formula:

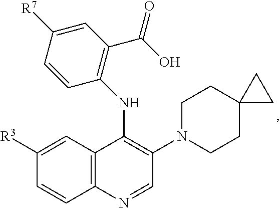

or a pharmaceutically acceptable salt thereof.

In some embodiments, the compound of Formula (XVI) has formula:

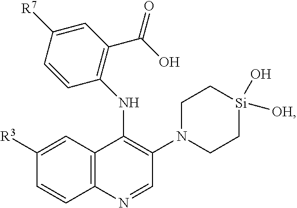

or a pharmaceutically acceptable salt thereof.

In some embodiments, the compound of Formula (XVI) has formula:

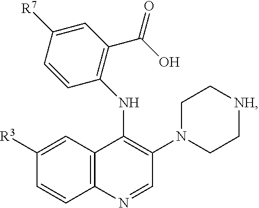

or a pharmaceutically acceptable salt thereof.

In some embodiments, the compound of Formula (XVI) has formula:

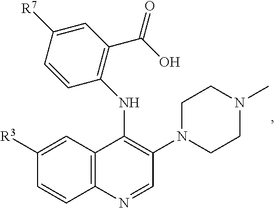

or a pharmaceutically acceptable salt thereof.

In some embodiments, the compound of Formula (XVI) has formula:

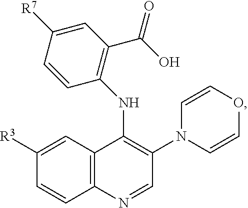

or a pharmaceutically acceptable salt thereof.

In some embodiments, the compound of Formula (XVI) has formula:

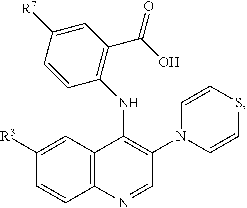

or a pharmaceutically acceptable salt thereof.

In some embodiments, the compound of Formula (XVI) has formula:

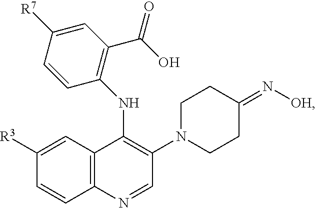

or a pharmaceutically acceptable salt thereof.

In some embodiments, R³ is selected from Cl, Br, and F. In some embodiments, R³ is Cl. In some embodiments, R³ is Br. In some embodiments, R³ is F.

In some embodiments, R⁷ is selected from halo, C₁₋₃ alkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (XVI) is selected from any one of the following compounds:

TABLE XVI

| | |
|---|---|
| 339A | 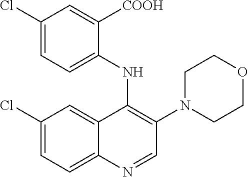 [SMILES: O=C(O)c1cc(Cl)ccc1Nc1c(N2CCOCC2)cnc2ccc(Cl)cc12] |
| 340A | 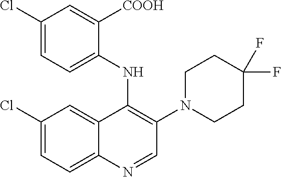 [SMILES: O=C(O)c1cc(Cl)ccc1Nc1c(N2CCC(F)(F)CC2)cnc2ccc(Cl)cc12] |
| 346A | 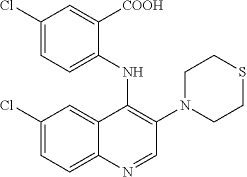 [SMILES: O=C(O)c1cc(Cl)ccc1Nc1c(N2CCSCC2)cnc2ccc(Cl)cc12] |
| 347A | 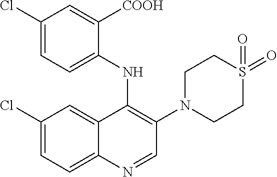 [SMILES: O=C(O)c1cc(Cl)ccc1Nc1c(N2CCS(=O)(=O)CC2)cnc2ccc(Cl)cc12] |
| BCH-361A | 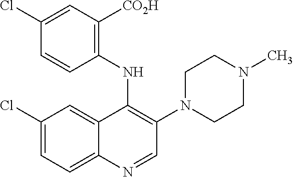 [SMILES: CN1CCN(c2cnc3ccc(Cl)cc3c2Nc2ccc(Cl)cc2C(=O)O)CC1] |
| BCH-362A | 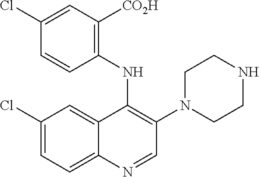 [SMILES: O=C(O)c1cc(Cl)ccc1Nc1c(N2CCNCC2)cnc2ccc(Cl)cc12] |
| | 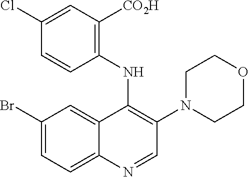 [SMILES: O=C(O)c1cc(Cl)ccc1Nc1c(N2CCOCC2)cnc2ccc(Br)cc12] |
| | 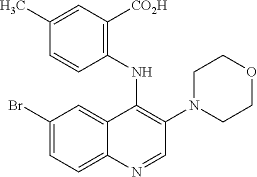 [SMILES: Cc1ccc(Nc2c(N3CCOCC3)cnc3ccc(Br)cc23)c(C(=O)O)c1] |
| | 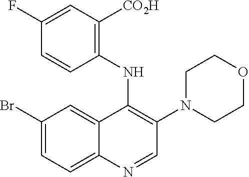 [SMILES: O=C(O)c1cc(F)ccc1Nc1c(N2CCOCC2)cnc2ccc(Br)cc12] |
| | 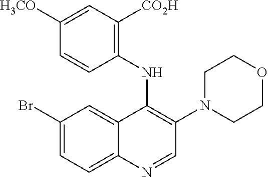 [SMILES: COc1ccc(Nc2c(N3CCOCC3)cnc3ccc(Br)cc23)c(C(=O)O)c1] |
| | 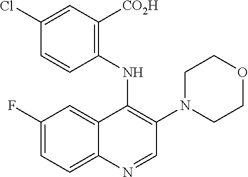 [SMILES: O=C(O)c1cc(Cl)ccc1Nc1c(N2CCOCC2)cnc2ccc(F)cc12] |
| | 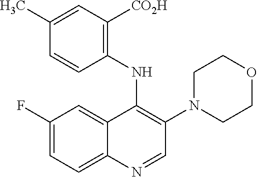 [SMILES: Cc1ccc(Nc2c(N3CCOCC3)cnc3ccc(F)cc23)c(C(=O)O)c1] |
| | 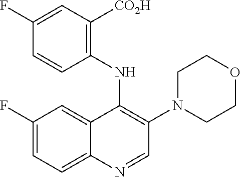 [SMILES: O=C(O)c1cc(F)ccc1Nc1c(N2CCOCC2)cnc2ccc(F)cc12] |
| | 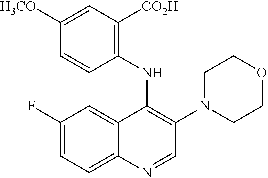 [SMILES: COc1ccc(Nc2c(N3CCOCC3)cnc3ccc(F)cc23)c(C(=O)O)c1] |
| | 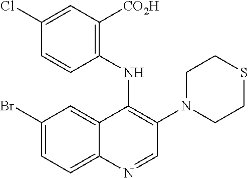 [SMILES: O=C(O)c1cc(Cl)ccc1Nc1c(N2CCSCC2)cnc2ccc(Br)cc12] |
| | 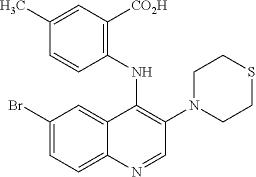 [SMILES: Cc1ccc(Nc2c(N3CCSCC3)cnc3ccc(Br)cc23)c(C(=O)O)c1] |
| | 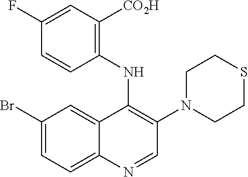 [SMILES: O=C(O)c1cc(F)ccc1Nc1c(N2CCSCC2)cnc2ccc(Br)cc12] |
| | 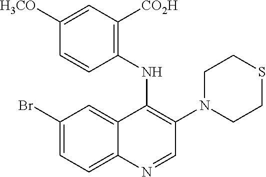 [SMILES: COc1ccc(Nc2c(N3CCSCC3)cnc3ccc(Br)cc23)c(C(=O)O)c1] |
| | 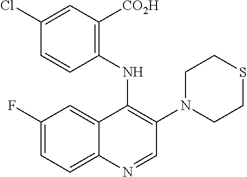 [SMILES: O=C(O)c1cc(Cl)ccc1Nc1c(N2CCSCC2)cnc2ccc(F)cc12] |
| | 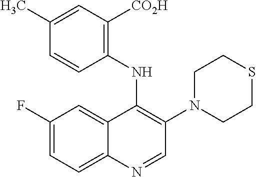 [SMILES: Cc1ccc(Nc2c(N3CCSCC3)cnc3ccc(F)cc23)c(C(=O)O)c1] |
| | 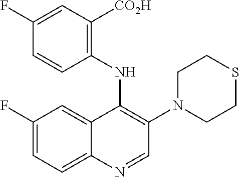 [SMILES: O=C(O)c1cc(F)ccc1Nc1c(N2CCSCC2)cnc2ccc(F)cc12] |
| | 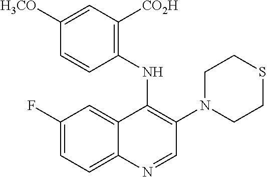 [SMILES: COc1ccc(Nc2c(N3CCSCC3)cnc3ccc(F)cc23)c(C(=O)O)c1] |
| | 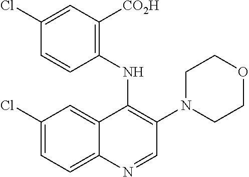 [SMILES: O=C(O)c1cc(Cl)ccc1Nc1c(N2CCOCC2)cnc2ccc(Cl)cc12] |
| | 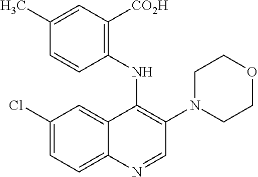 [SMILES: Cc1ccc(Nc2c(N3CCOCC3)cnc3ccc(Cl)cc23)c(C(=O)O)c1] |
| | 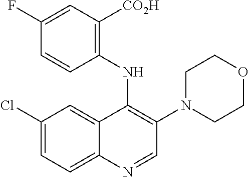 [SMILES: O=C(O)c1cc(F)ccc1Nc1c(N2CCOCC2)cnc2ccc(Cl)cc12] |
| | 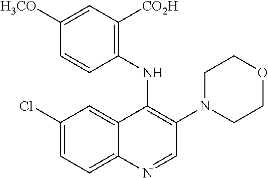 [SMILES: COc1ccc(Nc2c(N3CCOCC3)cnc3ccc(Cl)cc23)c(C(=O)O)c1] |
| | 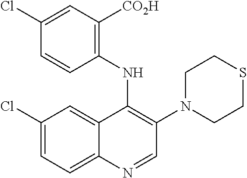 [SMILES: O=C(O)c1cc(Cl)ccc1Nc1c(N2CCSCC2)cnc2ccc(Cl)cc12] |
| | 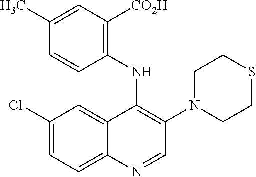 [SMILES: Cc1ccc(Nc2c(N3CCSCC3)cnc3ccc(Cl)cc23)c(C(=O)O)c1] |
| | 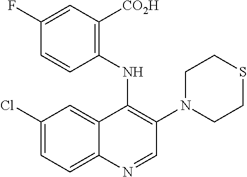 [SMILES: O=C(O)c1cc(F)ccc1Nc1c(N2CCSCC2)cnc2ccc(Cl)cc12] |
| | 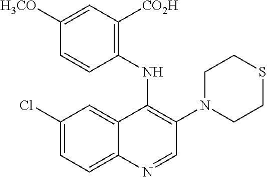 [SMILES: COc1ccc(Nc2c(N3CCSCC3)cnc3ccc(Cl)cc23)c(C(=O)O)c1] |
| BCH-376A | 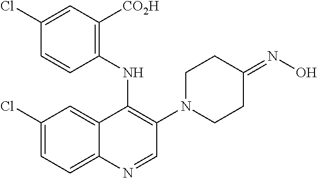 [SMILES: O=C(O)c1cc(Cl)ccc1Nc1c(N2CCC(=NO)CC2)cnc2ccc(Cl)cc12] |
| | 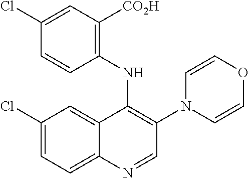 [SMILES: O=C(O)c1cc(Cl)ccc1Nc1c(N2C=COC=C2)cnc2ccc(Cl)cc12] |
| | 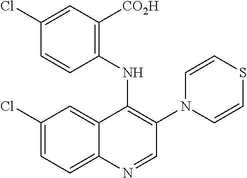 [SMILES: O=C(O)c1cc(Cl)ccc1Nc1c(N2C=CSC=C2)cnc2ccc(Cl)cc12] |
| BCH-393A | 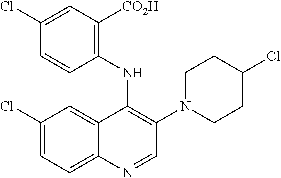 [SMILES: O=C(O)c1cc(Cl)ccc1Nc1c(N2CCC(Cl)CC2)cnc2ccc(Cl)cc12] |
| BCH-430A | 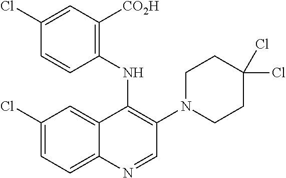 [SMILES: O=C(O)c1cc(Cl)ccc1Nc1c(N2CCC(Cl)(Cl)CC2)cnc2ccc(Cl)cc12] |
| BCH-392A | 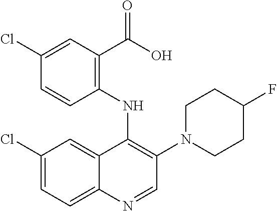 [SMILES: O=C(O)c1cc(Cl)ccc1Nc1c(N2CCC(F)CC2)cnc2ccc(Cl)cc12] |
| BCH-394A | 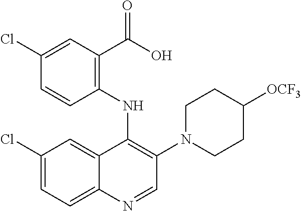 [SMILES: O=C(O)c1cc(Cl)ccc1Nc1c(N2CCC(OC(F)(F)F)CC2)cnc2ccc(Cl)cc12] |
| BCH-395A | 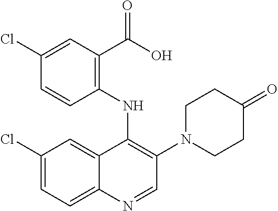 [SMILES: O=C1CCN(c2cnc3ccc(Cl)cc3c2Nc2ccc(Cl)cc2C(=O)O)CC1] |
| BCH-399A | 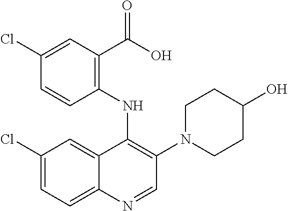 [SMILES: O=C(O)c1cc(Cl)ccc1Nc1c(N2CCC(O)CC2)cnc2ccc(Cl)cc12] |

or a pharmaceutically acceptable salt thereof.

In some embodiments, the present disclosure provides a compound of formula (XVII):

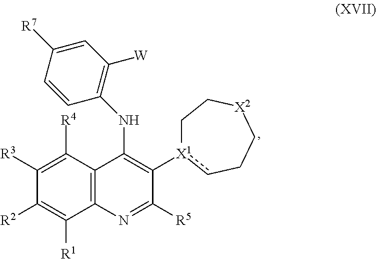

or a pharmaceutically acceptable salt thereof, wherein:
X¹ is N or CH when [symbol] is a sing e bond;
X¹ is C when [symbol] is a double bond;
X² is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo;

In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, X¹ is O. In some embodiments, X¹ is S.

In some embodiments, the compound of Formula (XVII) has formula:

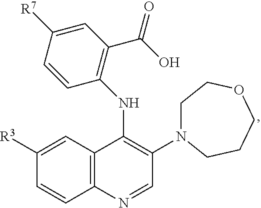

or a pharmaceutically acceptable salt thereof.

In some embodiments, the compound of Formula (XVII) has formula:

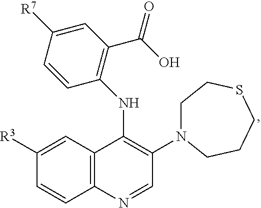

or a pharmaceutically acceptable salt thereof.

In some embodiments, the compound of Formula (XVII) has formula:

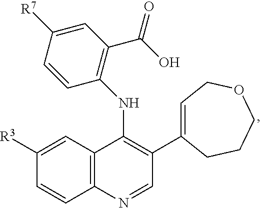

or a pharmaceutically acceptable salt thereof.

In some embodiments, the compound of Formula (XVII) has formula:

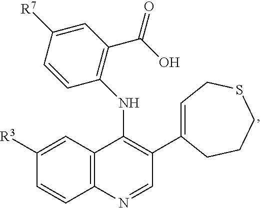

or a pharmaceutically acceptable salt thereof.

In some embodiments, R³ is selected from Cl, Br, and F. In some embodiments, R³ is Cl. In some embodiments, R³ is Br. In some embodiments, R³ is F.

In some embodiments, R⁷ is selected from halo, C₁₋₃ alkyl, and C₁₋₃ alkoxy.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, the compound of Formula (XVII) is selected from any one of the following compounds:

TABLE XVII

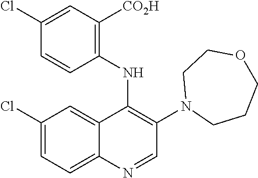 [SMILES: O=C(O)c1cc(Cl)ccc1Nc1c(N2CCCOCC2)cnc2ccc(Cl)cc12]

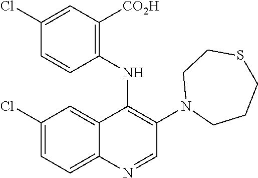 [SMILES: O=C(O)c1cc(Cl)ccc1Nc1c(N2CCCSCC2)cnc2ccc(Cl)cc12]

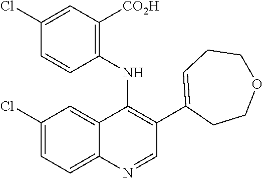 [SMILES: O=C(O)c1cc(Cl)ccc1Nc1c(C2=CCCOCC2)cnc2ccc(Cl)cc12]

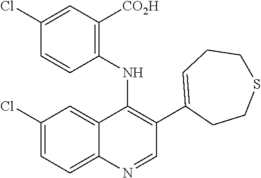 [SMILES: O=C(O)c1cc(Cl)ccc1Nc1c(C2=CCCSCC2)cnc2ccc(Cl)cc12]

or a pharmaceutically acceptable salt thereof.

In some embodiments, the present disclosure provides a compound of formula (XVIII):

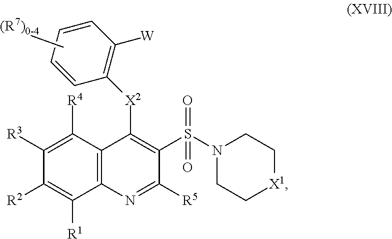

or a pharmaceutically acceptable salt thereof, wherein:
X¹ is selected from O and S;
X² is selected from CH₂, CHCH₃, and C(CH₃)₂;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

In some embodiments, X¹ is O. In some embodiments, X¹ is S. In some embodiments, X² is CH₂. In some embodiments, X² is CHCH₃. In some embodiments, X² is C(CH₃)₂.

In some embodiments, R¹, R², R⁴, and R⁵ are each independently selected from H, C₁₋₃ alkyl, C₁₋₃ alkoxy, C₁₋₄ haloalkyl, C₁₋₄ haloalkoxy, and halo; In some embodiments, R¹, R², R⁴, and R⁵ are each independently selected from H, C₁₋₃ alkyl, C₁₋₃ alkoxy, and halo.

In some embodiments, R¹, R², R⁴, and R⁵ are each independently selected from H and C₁₋₃ alkyl.

In some embodiments, W is C(O)OR⁸. In some embodiments, R⁸ is C₁₋₆ alkyl. In some embodiments, W is C(O)OH.

In some embodiments, W is a carboxylic acid bioisostere (e.g., any one of the carboxylic acid bioisostere groups described herein for Formula (I)).

In some embodiments, the compound of Formula (XVIII) has formula:

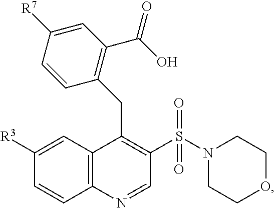

or a pharmaceutically acceptable salt thereof.

In some embodiments, the compound of Formula (XVIII) has formula:

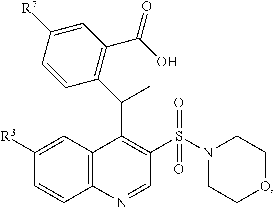

or a pharmaceutically acceptable salt thereof.

In some embodiments, the compound of Formula (XVIII) has formula:

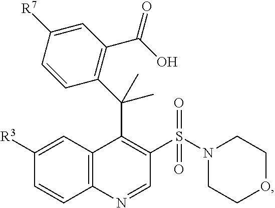

or a pharmaceutically acceptable salt thereof.

In some embodiments, R³ is selected from Cl, Br, and F.

In some embodiments, R³ is Cl. In some embodiments, R³ is Br. In some embodiments, R³ is F.

In some embodiments, R⁷ is halo. In some embodiments, R⁷ is selected from Cl, Br, and F. In some embodiments, R⁷ is Cl. In some embodiments, R⁷ is Br. In some embodiments, R⁷ is F.

In some embodiments, R⁷ is C₁₋₃ alkyl. In some embodiments, R⁷ is methyl.

In some embodiments, R⁷ is C₁₋₃ alkoxy. In some embodiments, R⁷ is methoxy.

In some embodiments, R⁷ is B(OH)₂. In some embodiments, R⁷ is OH. In some embodiments, R⁷ is CN. In some embodiments, R⁷ is C₁₋₃ haloalkyl. In some embodiments, R⁷ is HO—C₁₋₃ haloalkyl. In some embodiments, R⁷ is aminosulfonyl. In some embodiments, R⁷ is C₁₋₃ haloalkylcarbonyl. In some embodiments, R⁷ is C₁₋₃ alkylcarbonyl. In some embodiments, R⁷ is carbamyl.

In some embodiments, R³ is F and R⁷ is Cl. In some embodiments, R³ is F and R⁷ is C₁₋₃ alkyl. In some embodiments, R³ is F and R⁷ is F. In some embodiments, R³ is F and R⁷ is C₁₋₃ alkoxy. In some embodiments, R³ is Cl and R⁷ is Cl. In some embodiments, R³ is Cl and R⁷ is F. In some embodiments, R³ is Cl and R⁷ is C₁₋₃ alkyl. In some embodiments, R³ is Cl and R⁷ is C₁₋₃ alkoxy. In some embodiments, R³ is Br and R⁷ is Cl. In some embodiments, R³ is Br and R⁷ is C₁₋₃ alkyl. In some embodiments, R³ is Br and R⁷ is F. In some embodiments, R³ is Br and R⁷ is C₁₋₃ alkoxy.

In some embodiments, the compound of Formula (XVIII) is selected from any one of the following compounds:

TABLE XVIII

| | |
|---|---|
| 337A | 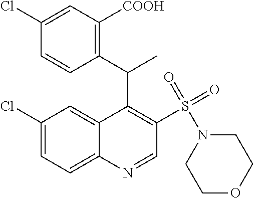 [SMILES: CC(c1ccc(Cl)cc1C(=O)O)c1c(S(=O)(=O)N2CCOCC2)cnc2ccc(Cl)cc12] |
| 336A | 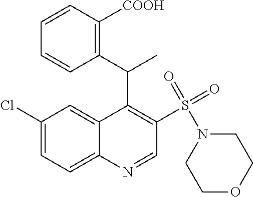 [SMILES: CC(c1ccccc1C(=O)O)c1c(S(=O)(=O)N2CCOCC2)cnc2ccc(Cl)cc12] |
| 333A | 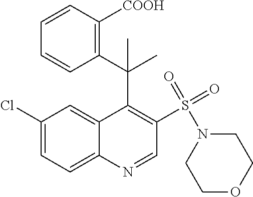 [SMILES: CC(C)(c1ccccc1C(=O)O)c1c(S(=O)(=O)N2CCOCC2)cnc2ccc(Cl)cc12] |
| 334A | 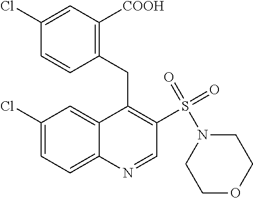 [SMILES: O=C(O)c1cc(Cl)ccc1Cc1c(S(=O)(=O)N2CCOCC2)cnc2ccc(Cl)cc12] |
| 335A | 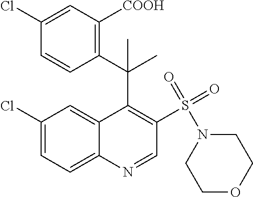 [SMILES: CC(C)(c1ccc(Cl)cc1C(=O)O)c1c(S(=O)(=O)N2CCOCC2)cnc2ccc(Cl)cc12] |

or a pharmaceutically acceptable salt thereof.

In some embodiments, the present disclosure provides a compound selected from any one of the following compounds:

TABLE 1A

| | |
|---|---|
| BCH- 87A-Cl | 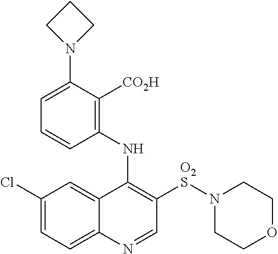 [SMILES: O=C(O)c1c(Nc2c(S(=O)(=O)N3CCOCC3)cnc3ccc(Cl)cc23)cccc1N1CCC1] |
| BCH- 91A-Cl | 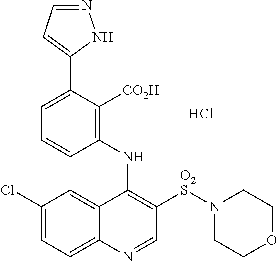 [SMILES: O=C(O)c1c(Nc2c(S(=O)(=O)N3CCOCC3)cnc3ccc(Cl)cc23)cccc1-c1ccn[nH]1 ; Cl] |
| BCH-261A | 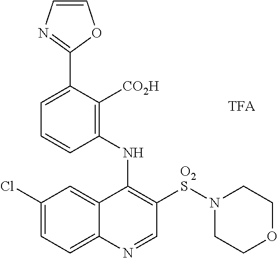 [SMILES: O=C(O)c1c(Nc2c(S(=O)(=O)N3CCOCC3)cnc3ccc(Cl)cc23)cccc1-c1ncco1 ; "TFA"] |
| BCH-124A | 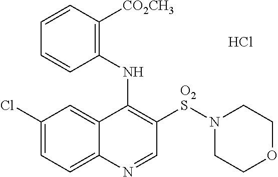 [SMILES: COC(=O)c1ccccc1Nc1c(S(=O)(=O)N2CCOCC2)cnc2ccc(Cl)cc12 ; Cl] |
| BCH-345A | 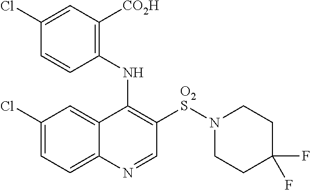 [SMILES: O=C(O)c1cc(Cl)ccc1Nc1c(S(=O)(=O)N2CCC(F)(F)CC2)cnc2ccc(Cl)cc12] |
| BCH-324A | 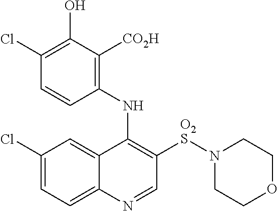 [SMILES: O=C(O)c1c(Nc2c(S(=O)(=O)N3CCOCC3)cnc3ccc(Cl)cc23)ccc(Cl)c1O] |
| BCH-220A | 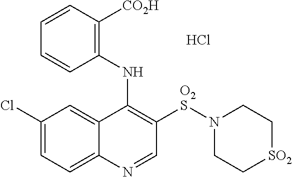 [SMILES: O=C(O)c1ccccc1Nc1c(S(=O)(=O)N2CCS(=O)(=O)CC2)cnc2ccc(Cl)cc12 ; Cl] |
| BCH-125A | 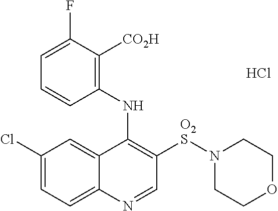 [SMILES: O=C(O)c1c(F)cccc1Nc1c(S(=O)(=O)N2CCOCC2)cnc2ccc(Cl)cc12 ; Cl] |
| BCH- 127A-1 | 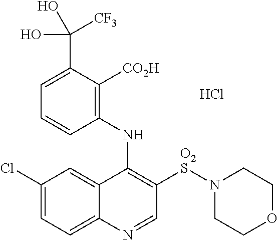 [SMILES: O=C(O)c1c(Nc2c(S(=O)(=O)N3CCOCC3)cnc3ccc(Cl)cc23)cccc1C(O)(O)C(F)(F)F ; Cl] |
| BCH- 128A-BR | 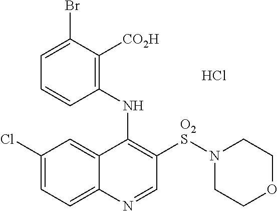 [SMILES: O=C(O)c1c(Br)cccc1Nc1c(S(=O)(=O)N2CCOCC2)cnc2ccc(Cl)cc12 ; Cl] |
| BCH-131A | 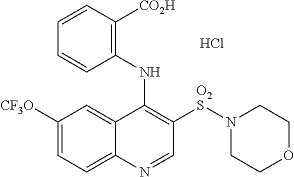 |
| BCH-233A | 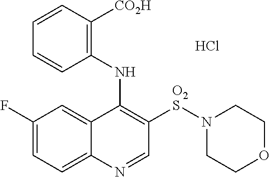 [SMILES: O=C(O)c1ccccc1Nc1c(S(=O)(=O)N2CCOCC2)cnc2ccc(F)cc12 ; Cl] |
| BCH-234A | 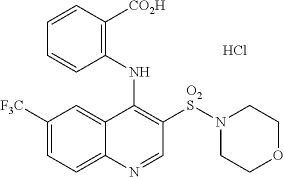 [SMILES: O=C(O)c1ccccc1Nc1c(S(=O)(=O)N2CCOCC2)cnc2ccc(C(F)(F)F)cc12 ; Cl] |
| BCH-235A | 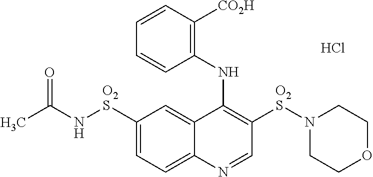 [SMILES: CC(=O)NS(=O)(=O)c1ccc2ncc(S(=O)(=O)N3CCOCC3)c(Nc3ccccc3C(=O)O)c2c1 ; Cl] |
| BCH-236A | 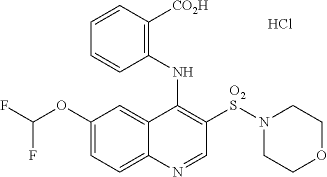 [SMILES: O=C(O)c1ccccc1Nc1c(S(=O)(=O)N2CCOCC2)cnc2ccc(OC(F)F)cc12 ; Cl] |
| BCH-249A | 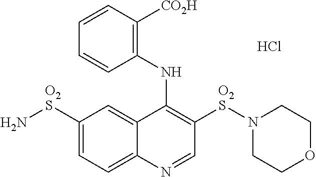 [SMILES: NS(=O)(=O)c1ccc2ncc(S(=O)(=O)N3CCOCC3)c(Nc3ccccc3C(=O)O)c2c1 ; Cl] |
| BCH-250A | 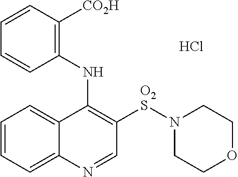 [SMILES: O=C(O)c1ccccc1Nc1c(S(=O)(=O)N2CCOCC2)cnc2ccccc12 ; Cl] |
| BCH-321A | 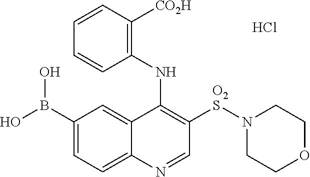 [SMILES: O=C(O)c1ccccc1Nc1c(S(=O)(=O)N2CCOCC2)cnc2ccc(B(O)O)cc12 ; Cl] |
| BCH-322A | 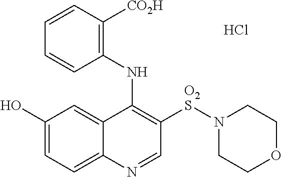 [SMILES: O=C(O)c1ccccc1Nc1c(S(=O)(=O)N2CCOCC2)cnc2ccc(O)cc12 ; Cl] |
| BCH-292A | 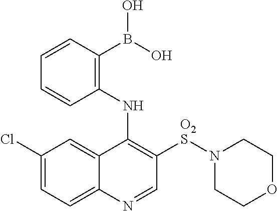 [SMILES: O=S(=O)(c1cnc2ccc(Cl)cc2c1Nc1ccccc1B(O)O)N1CCOCC1] |
| BCH-129A | 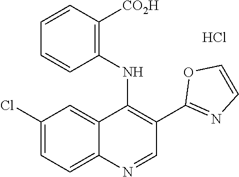 [SMILES: O=C(O)c1ccccc1Nc1c(-c2ncco2)cnc2ccc(Cl)cc12 ; Cl] |
| BCH-130A | 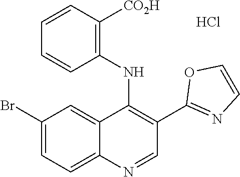 [SMILES: O=C(O)c1ccccc1Nc1c(-c2ncco2)cnc2ccc(Br)cc12 ; Cl] |
| BCH-211A | 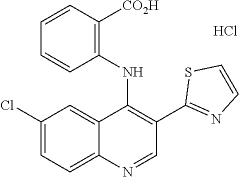 [SMILES: O=C(O)c1ccccc1Nc1c(-c2nccs2)cnc2ccc(Cl)cc12 ; Cl] |
| BCH-212A | 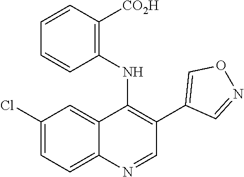 [SMILES: O=C(O)c1ccccc1Nc1c(-c2cnoc2)cnc2ccc(Cl)cc12] |

or a pharmaceutically acceptable salt thereof.

In some embodiments, the present disclosure provides a compound selected from any one of the following compounds:

TABLE 2A

| | |
|---|---|
| BCH-248A | 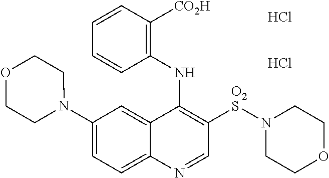 [SMILES: O=C(O)c1ccccc1Nc1c(S(=O)(=O)N2CCOCC2)cnc2ccc(N3CCOCC3)cc12 ; Cl ; Cl] |
| BCH-232A | 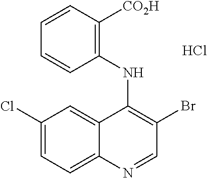 [SMILES: O=C(O)c1ccccc1Nc1c(Br)cnc2ccc(Cl)cc12 ; Cl] |
| BCH-204A-BP | 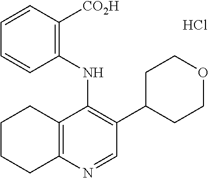 [SMILES: O=C(O)c1ccccc1Nc1c(C2CCOCC2)cnc2c1CCCC2 ; Cl] |
| BCH-135A | 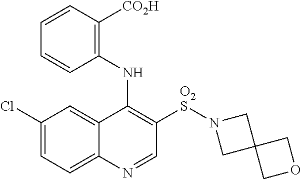 [SMILES: O=C(O)c1ccccc1Nc1c(S(=O)(=O)N2CC3(COC3)C2)cnc2ccc(Cl)cc12] |
| BCH-135A-BP | 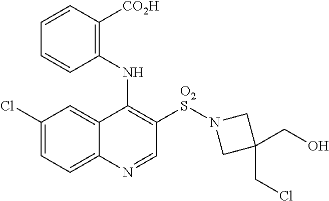 [SMILES: O=C(O)c1ccccc1Nc1c(S(=O)(=O)N2CC(CO)(CCl)C2)cnc2ccc(Cl)cc12] |
| BCH-371A | 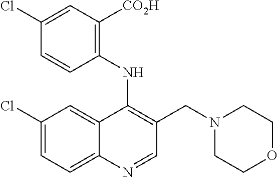 [SMILES: O=C(O)c1cc(Cl)ccc1Nc1c(CN2CCOCC2)cnc2ccc(Cl)cc12] |
| BCH-428A | 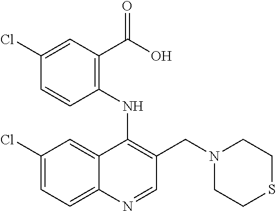 [SMILES: O=C(O)c1cc(Cl)ccc1Nc1c(CN2CCSCC2)cnc2ccc(Cl)cc12] |
| BCH-372A | 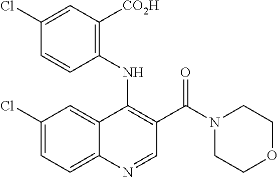 [SMILES: O=C(O)c1cc(Cl)ccc1Nc1c(C(=O)N2CCOCC2)cnc2ccc(Cl)cc12] |
| | 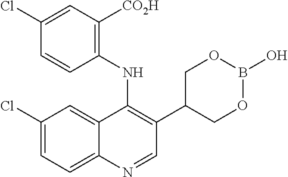 [SMILES: O=C(O)c1cc(Cl)ccc1Nc1c(C2COB(O)OC2)cnc2ccc(Cl)cc12] |
| | 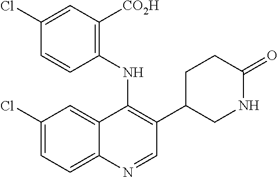 [SMILES: O=C1CCC(c2cnc3ccc(Cl)cc3c2Nc2ccc(Cl)cc2C(=O)O)CN1] |
| 350A | 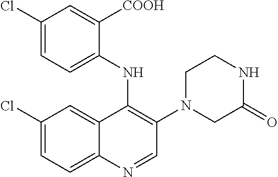 [SMILES: O=C1CN(c2cnc3ccc(Cl)cc3c2Nc2ccc(Cl)cc2C(=O)O)CCN1] |
| | 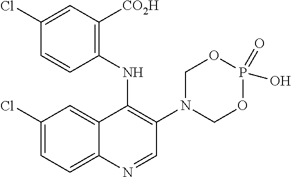 [SMILES: O=C(O)c1cc(Cl)ccc1Nc1c(N2COP(=O)(O)OC2)cnc2ccc(Cl)cc12] |
| | 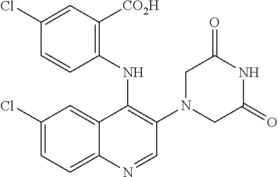 [SMILES: O=C1CN(c2cnc3ccc(Cl)cc3c2Nc2ccc(Cl)cc2C(=O)O)CC(=O)N1] |

or a pharmaceutically acceptable salt thereof

In some embodiments, the present disclosure provides a compound selected from any one of the following compounds:

TABLE 2B

| | |
|---|---|
| BCH-132A | 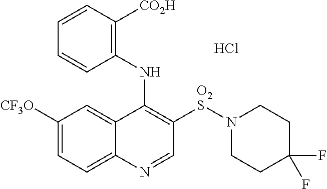 |
| BCH-133A | 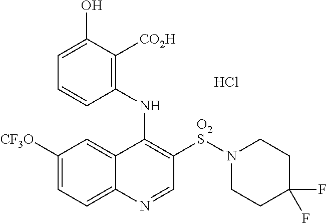 |

or a pharmaceutically acceptable salt thereof

In some embodiments, the present disclosure provides a compound selected from any one of the following compounds:

TABLE 2C

| | |
|---|---|
| BCH-123A | 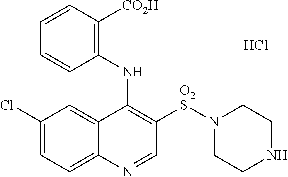 [SMILES: O=C(O)c1ccccc1Nc1c(S(=O)(=O)N2CCNCC2)cnc2ccc(Cl)cc12 ; Cl] |
| BCH-123A-CBZ | 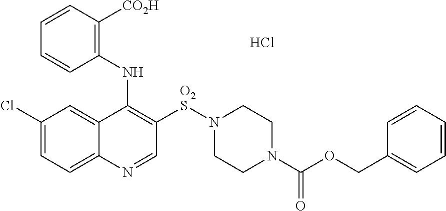 [SMILES: O=C(O)c1ccccc1Nc1c(S(=O)(=O)N2CCN(C(=O)OCc3ccccc3)CC2)cnc2ccc(Cl)cc12 ; Cl] |

or a pharmaceutically acceptable salt thereof

In some embodiments, the present disclosure provides a compound selected from any one of the following compounds:

TABLE 2D

| | |
|---|---|
| BCH-328A | 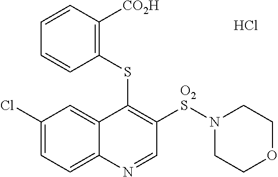 [SMILES: O=C(O)c1ccccc1Sc1c(S(=O)(=O)N2CCOCC2)cnc2ccc(Cl)cc12 ; Cl] |
| BCH-330A | 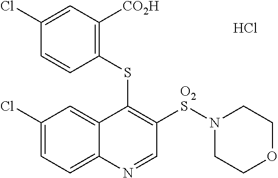 [SMILES: O=C(O)c1cc(Cl)ccc1Sc1c(S(=O)(=O)N2CCOCC2)cnc2ccc(Cl)cc12 ; Cl] |

or a pharmaceutically acceptable salt thereof.

In some embodiments, the present disclosure provides a compound selected from any one of the following compounds:

TABLE 2E

| | |
|---|---|
| 326A | 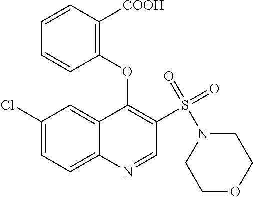 [SMILES: O=C(O)c1ccccc1Oc1c(S(=O)(=O)N2CCOCC2)cnc2ccc(Cl)cc12] |
| 327A | 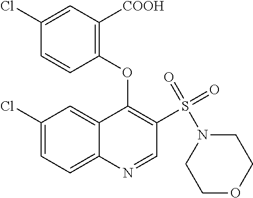 [SMILES: O=C(O)c1cc(Cl)ccc1Oc1c(S(=O)(=O)N2CCOCC2)cnc2ccc(Cl)cc12] |

or a pharmaceutical y acceptable salt thereof.

As used herein, the term “pharmaceutically acceptable salt” refers to a salt that is formed between an acid and a basic group of the compound, such as an amino functional group, or between a base and an acidic group of the compound, such as a carboxyl functional group. In some embodiments, the compound is a pharmaceutically acceptable acid addition salt. In some embodiments, acids commonly employed to form pharmaceutically acceptable salts of the therapeutic compounds described herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, 3-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the therapeutic compounds described herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compound of Formulae (I)-(IV), or a pharmaceutically acceptable salt thereof, is substantially isolated.

### Methods of Making

Compounds of any one of Formulae disclosed herein, including salts thereof, can be prepared using known organic synthesis techniques and can be synthesized according to any of numerous possible synthetic routes. A person skilled in the art knows how to select and implement appropriate synthetic protocols, and appreciates that a broad repertoire of synthetic organic reactions is available to be potentially employed in synthesizing compounds provided herein.

Suitable synthetic methods of starting materials, intermediates and products can be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Methods of Use

### Modulation of Telomerase RNA Component (TERC)

Telomerase has been a therapeutic target of great interest for over two decades, based on its activity in numerous cancers. The telomerase RNA component (TERC) contains a box H/ACA domain at its 3′ end, a motif that is functionally separable from the template domain and dispensable for telomerase activity in vitro. In vivo, the H/ACA motif is bound by a heterotrimer of dyskerin. NOP10, and NHP2 which stabilize TERC, and also by TCAB1, which is responsible for localizing the telomerase complex to Cajal bodies (I-Venteicher, A. S. et al. A human telomerase holoenzyme protein required for Cajal body localization and telomere synthesis. Science 323, 644-8 (2009)). Disruption of any of these interactions can also compromise telomere maintenance and cause telomere disease (Mitchell, J. R., Wood, E. & Collins, K. A telomerase component is defective in the human disease dyskeratosis congenita. Nature 402, 551-5 (1999); Vulliamy, T. et al. Mutations in the telomerase component NHP2 cause the premature ageing syndrome dyskeratosis congenita. Proceedings of the National Academy of Sciences of the United States of America 105, 8073-8 (2008); Walne, A. J. et al. Genetic heterogeneity in autosomal recessive dyskeratosis congenita with one subtype due to mutations in the telomerase-associated protein NOP10. Human molecular genetics 16, 1619-29 (2007)). The H/ACA motif serve as guides for pseudouridylation of other RNAs by dyskerin (Kiss, T., Fayet-Lebaron, E. & Jady, B. E. Box H/ACA small ribonucleoproteins. Molecular cell 37, 597-606 (2010)).

Increasing telomerase activity can be beneficial in several degenerative and age-related disorders. Conversely, inhibiting telomerase activity would be of significant utility for the treatment of cancer and disorders in which hyper-proliferative cells depend on telomerase for self-renewal.

### Modulation of Poly(A) Specific Ribonuclease (PARN)

PARN is known as a 3′-5′ exoribonuclease responsible for degradation of the poly(A) tails of eukaryotic mRNAs, which is a rate-limiting step in mRNA turnover (Korner, C. G. & Wahle, E. Poly(A) tail shortening by a mammalian poly(A)-specific 3′-exoribonuclease. The Journal of biological chemistry 272, 10448-56 (1997)). PARN is stimulated by presence of a m7G-cap, and requires a minimal substrate of adenosine di- or tri-nucleotides—in other words, oligo(A) rather than strictly poly(A). PARN is a widely-expressed cap-dependent, poly(A) deadenylase with a canonical role in regulating global mRNA levels during development, and additional, more specialized functions including end-trimming of the Dicer-independent microRNA (miR)-451 and deadenylation of small nucleolar (sno)RNAs. PARN loss-of-function mutations are implicated in idiopathic pulmonary fibrosis and dyskeratosis congenita. The disclosure provides methods and agents that modulate the level or activity of human PARN. The nucleotide sequence of human PARN is NM_002582 and the amino acid sequence of PARN is O95453 (Table 1). Variants of the nucleotide sequence and the amino acid sequence are also shown in Table 1.

TABLE 1
Accession numbers for genes, RNA and proteins

| | | Nucleotide | Protein ID(s) |
|---|---|---|---|
| | | sequence(s) and | and variants |
| | | variants therein | therein |
| | | (RefSeq unless | (Uniprot unless |
| | | otherwise | otherwise |
| Gene | Ensembl Gene ID | indicated) | indicated) |
| TERC | ENSG00000270141 | NR_001566 | N/A |
| PARN | ENSG00000140694 | NM_002582 | O95453 |
| | | NM_001242992 | |
| TRF4-2 | ENSG00000121274 | NM_001134477 | QBNDF8 |
| a.k.a. | | NM_001040284 | |
| PAPD5 | | NM_001040285 | H3BQM0 |
| | | FR872509.1 | CCB84642.1 |
| | | (GenBank) | (GenBank) |

### PAP Associated Domain Containing 5 (PAPD5)

PAPD5, also known as Topoisomerase-Related Function Protein 4-2 (TRF4-2), also known as TUT3, also known as GLD4, also known as TENT4B, is one of the seven members of the family of noncanonical poly(A) polymerases in human cells. PAPD5 has been shown to act as a polyadenylase on abnormal pre-ribosomal RNAs in vivo in a manner analogous to degradation-mediating polyadenylation by the non-canonical poly(A) polymerase Trf4p in yeast. PAPD5 is also involved in the uridylation-dependent degradation of histone mRNAs.

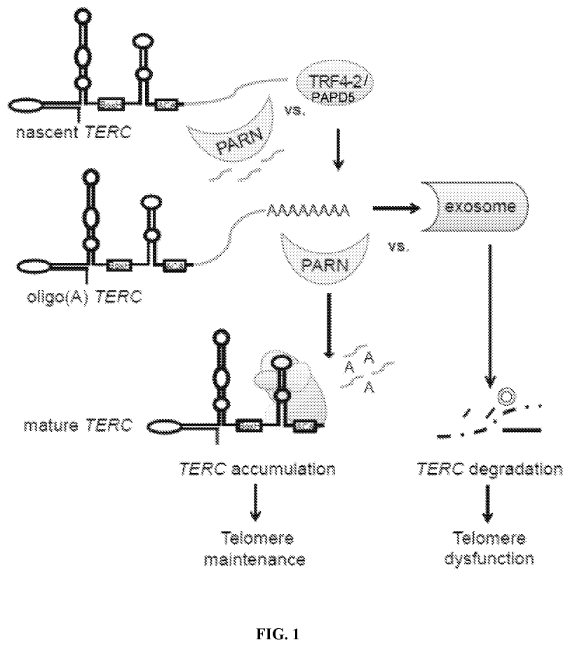

Both PARN and PAPD5 are involved in the 3′-end maturation of the telomerase RNA component (TERC). Patient cells, fibroblast cells as well as converted fibroblasts (I-IPS cells) in which PARN is disrupted show decreased levels of TERC which can be restored by decreasing levels or activities of PAPD5. Deep sequencing of TERC RNA 3′ termini or ends, reveals that PARN and PAPD5 are critically important for processing of post-transcriptionally acquired oligo(A) tails that target nuclear RNAs for degradation. Diminished TERC levels and the increased oligo(A) forms of TERC are normalized by restoring PARN or inhibiting PAPD5. The disclosure reveals PARN and PAPD5 as important players in the regulation and biogenesis of TERC (FIG. 1). FIG. 1 shows 3′ ends of nascent TERC RNA are subject to PAPD5-mediated oligo-adenylation, which targets transcripts for degradation by the exosome. PARN counteracts the degradation pathway by removing oligo(A) tails and/or trimming genomically-encoded bases (green) of nascent TERC to yield a mature 3′ end. Mature TERC is protected from further oligo-adenylation and exonucleolytic processing, possibly by the dyskerin/NOP10/NHP2/GAR1 complex, and assembles into the telomerase holoenzyme to maintain telomeres. PARN deficiency tips the balance in favor of degradation, leading to reduced TERC levels and telomere dysfunction. Thus, the disclosure also provides compounds and methods that modulate the level or activity of human PAPD5. The nucleotide sequence of human PAPD5 used is FR872509.1, and the amino acid sequence is CCB84642.1 (Table 1). Variants of the nucleotide sequence and the amino acid sequence are also shown in Table 1. The amino acid sequence of PAPD5 used is shown below:

PAPD5 (TRF4-2) (CCB84642.1)
(SEQ ID NO: 1)
MYRSGERLLG SHALPAEQRD FLPLETTNNN NNHHQPGAWA RRAGSSASSP PSASSSPHPS
AAVPAADPAD SASGSSNKRK RDNKASTYGL NYSLLQPSGG RAAGGGRADG GGVVYSGTPW
KRRNYNQGVV GLHEEISDFY EYMSPRPEEE KMRMEVVNRI ESVIKELWPS ADVQIFGSFK
TGLYLPTSDI DLVVFGKWEN LPLWTLEEAL RKHKVADEDS VKVLDKATVP IIKLTDSFTE
VKVDISFNVQ NGVRAADLIK DFTKKYPVLP YLVLVLKQFL LQRDLNEVFT
GGIGSYSLFL MAVSFLQLHP REDACIPNTN YGVLLIEFFE LYGRHFNYLK
TGIRIKDGGS YVAKDEVQKN
MLDGYRPSML YIEDPLQPGN DVGRSSYGAM QVKQAFDYAY VVLSHAVSPI AKYYPNNETE
SILGRIIRVT DEVATYRDWI SKQWGLKNRP EPSCNGNGVT LIVDTQQLDK CNNNLSEENE
ALGKCRSKTS ESLSKHSSNS SSGPVSSSSA TQSSSSDVDS DATPCKTPKQ LLCRPSTGNR
VGSQDVSLES SQAVGKMQST QTTNTSNSTN KSQHGSARLF RSSSKGFQGT TQTSHGSLMT
NKQHQGKSNN QYYHGKKRKH KRDAPLSDLC R

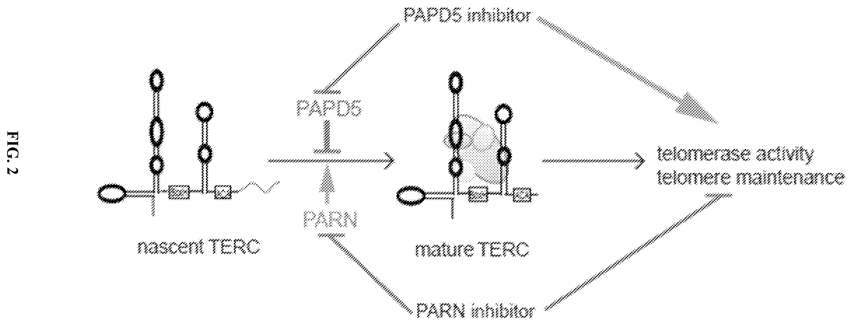

FIG. 2 is a diagram demonstrating the reciprocal regulation of TERC levels by PAPD5 and PARN, and the potential for therapeutic manipulation of telomerase in degenerative or malignant disorders. As shown in FIG. 2, a PAPD5 inhibitor can inhibit PAPD5-mediated oligo-adenylation, which targets nascent TERC RNA for degradation by the exosome, thus increases the level or activity of TERC. In contrast, as PARN counteracts the degradation pathway by removing oligo(A) tails and/or trimming genomically-encoded bases of nascent TERC to yield a mature 3′ end, PARN inhibitor will decrease the level or activity of TERC. In addition, increasing the level or activity of PARN can increase the level or activity of TERC, and increasing the level or activity of PAPD5 can decrease the level or activity of TERC.

In one aspect, the present disclosure provides compounds and associated methods of modulating TERC levels in cells. The cells can be, e.g., primary human cells, stem cells, induced pluripotent cells, fibroblasts, etc. In some embodiments, the cells are within a subject (e.g., a human subject). Therefore, the present disclosure provides methods modulating TERC levels in cells in vivo. In some embodiments, the cells can be isolated from a sample obtained from the subject, e.g., the cells can be derived from any part of the body including, but not limited to, skin, blood, and bone marrow. The cells can also be cultured in vitro using routine methods with commercially available cell reagents (e.g., cell culture media). In some embodiments, the cells are obtained from a subject, having a telomere disease, being at risk of developing a telomere disease, or being suspected of having a telomere disease. In some embodiments, the subject has no overt symptoms.

The level or activity of TERC can be determined by various means, e.g., by determining the size of telomere in the cell, by determining the stability of TERC, by determining the amount of RNA, by measuring the activity of telomerase function, and/or by measuring oligo-adenylated (oligo(A)) forms of TERC. TERC stability can be assessed, e.g., by measuring the TERC decay rates. Oligo-adenylated (oligo(A)) forms of TERC can be measured, e.g., using rapid amplification of cDNA ends (RACE) coupled with targeted deep sequencing (e.g., at the TERC 3′ end) to detect oligo-adenylated (oligo(A)) forms of TERC. The size of a telomere can be measured, e.g., using Flow-fluorescent in-situ hybridization (Flow-FISH) technique.

In some embodiments, the modulation of endogenous TERC is performed. Such methods can include, e.g., altering telomerase activity, e.g., increasing or decreasing telomerase activity. The methods can involve reducing RNA expression in cells, e.g., non-coding RNA in TERC. Telomerase activity can be, e.g., regulated by modulating TERC levels by contacting cells with test compounds known to modulate protein synthesis. The methods may involve targeting post-processing activity of the endogenous TERC locus. These methods involve manipulating TERC including identifying subjects with genetic mutation (e.g., mutation in PARN), isolating cells (e.g., fibroblast), and treating cells with agents that modulate TERC levels. The methods may also involve manipulating TERC including identifying subjects with genetic mutation (e.g., mutation in PARN) and treating the subject with agents that modulate TERC levels. Subject with genetic mutation (e.g., PARN mutation) may be identified by any diagnostic means generally known in the art for that purpose.

The present disclosure shows that TERC levels are modulated at the post-transcriptional level. Thus, in one aspect, methods of modulating the level or activity of TERC involve modulating the level or activity of PARN and PAPD5.

In some embodiments, the methods involve an agent that modulates the level or activity of PARN, thereby altering the level or activity of TERC. In some cases, the agent increases the level or activity of PARN. Alternatively, the agent decreases the level or activity of PARN. In some embodiments, the methods involve an agent that modulates the level or activity of PAPD5, thereby altering the level or activity of TERC. In some embodiments, the agent increases the level or activity of PAPD5. Alternatively, the agent decreases the level or activity of PAPD5 (e.g., PAPD5 inhibitors). In some embodiments, the agent is any one of compounds described herein.

Accordingly, the present application provides compounds that modulate TERC levels and are thus useful in treating a broad array of telomere diseases or disorders associated with telomerase dysfunction, e.g., dyskeratosis congenita, aplastic anemia, pulmonary fibrosis, idiopathic pulmonary fibrosis, hematological disorder, hepatic disease (e.g., chronic liver disease), and cancer, e.g., hematological cancer and hepatocarcinoma, etc.

In some embodiments, in order to successfully treat a telomere disease, a therapeutic agent has to selectively inhibit PAPD5, while not inhibiting PARN or other polynucleotide polymerases. A PAPD5 inhibitor that is not selective and concurrently inhibits other polymerases, may not be useful in treating telomere diseases; that is, the fact that a compound is a PAPD5 inhibitor (e.g., non-selective inhibitor) is not indicative of its usefulness in prevention and treatment of telomere diseases. The selectivity to PAPD5 as opposed to other polymerases is required for potency. In some embodiments, the compounds of the present application are selective and specific inhibitors of PAPD5 and do not inhibit PARN or other polymerases.

In some embodiments, it was surprisingly discovered that in order to successfully treat a telomere disease, a therapeutic agent has to be a selective inhibitor of PAPD5. In other words, a successful therapeutic agent has to inhibit PAPD5 while not substantially inhibiting PARN and/or other polynucleotide polymerases. In some embodiments, a PAPD5 inhibitor that is not selective to PAPD5 and concurrently inhibits other polymerases, may not be useful in treating telomere diseases; that is, the fact that a compound is a PAPD5 inhibitor (e.g., non-selective inhibitor) is not indicative of its usefulness in prevention and treatment of telomere diseases. The selectivity to PAPD5 as opposed to other polymerases is required for potency. In some embodiments, the compounds of the present application are selective and specific inhibitors of PAPD5 and do not substantially inhibit PARN or other polymerases.

### Telomere Diseases

Telomere diseases or disorders associated with telomerase dysfunction are typically associated with changes in the size of telomere. Many proteins and RNA components are involved in the telomere regulatory pathway, including TERC, PARN and PAPD5 (also known as TRF4-2). FIGS. 1 and 2 show how these proteins or RNA components work in the regulatory pathway and how they are related to telomere diseases.

Among these telomere diseases is dyskeratosis congenita (DC), which is a rare, progressive bone marrow failure syndrome characterized by the triad of reticulated skin hyperpigmentation, nail dystrophy, and oral leukoplakia. Early mortality is often associated with bone marrow failure, infections, fatal pulmonary complications, or malignancy. Short-term treatment options for bone marrow failure in patients include anabolic steroids (e.g., oxymetholone), granulocyte macrophage colony-stimulating factor, granulocyte colony-stimulating factor, and erythropoietin. Other treatments include hematopoietic stem cell transplantation (SCT).

Idiopathic pulmonary fibrosis is a chronic and μLtimately fatal disease characterized by a progressive decline in lung function. In some appropriate cases, the following agents are used to treat idiopathic pulmonary fibrosis: nintedanib, a tyrosine kinase inhibitor that targets multiple tyrosine kinases, including vascular endothelial growth factor, fibroblast growth factor, and PDGF receptors; and pirfenidone. Other treatments include lung transplantation. In some cases, lung transplantation for idiopathic pulmonary fibrosis (I-IPF) has been shown to confer a survival benefit over medical therapy.

Generally, a method of treating a telomere disease includes administering a therapeutically effective amount of a compound described herein, to a subject who is in need of, or who has been determined to be in need of, such treatment.

In some embodiments, the disorder associated with telomere or telomerase dysfunction is dyskeratosis congenita, aplastic anemia, myelodysplastic syndrome, pulmonary fibrosis, interstitial lung disease, hematological disorder, liver disease or hepatic fibrosis.

In some embodiments, the disorder associated with telomere or telomerase dysfunction is dyskeratosis congenita, aplastic anemia, pulmonary fibrosis, myelodysplastic syndrome, idiopathic pulmonary fibrosis, hematological disorder, or hepatic fibrosis.

### Cancer

The present disclosure also provides compounds, compositions, and methods for treating pre-leukemic conditions, pre-cancerous conditions, dysplasia and/or cancers. Pre-leukemic conditions include, e.g., Myelodysplastic syndrome, and smoldering leukemia. Dysplasia refers to an abnormality of development or an epithelial anomaly of growth and differentiation, including e.g., hip dysplasia, fibrous dysplasia, and renal dysplasia, Myelodysplastic syndromes, and dysplasia of blood-forming cells.

A precancerous condition or premalignant condition is a state of disordered morphology of cells that is associated with an increased risk of cancer. If left untreated, these conditions may lead to cancer. Such conditions are can be dysplasia or benign neoplasia.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells.

Many cancer cells have abnormal telomeres. Thus, treatments described herein (e.g., PAPD5 inhibitors) can also be used to treat cancers. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

In some embodiments, the methods described herein are used for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. Cancers treatable using the methods described herein are cancers that have increased levels of TERC, an increased expression of genes such as TERC and/or TERT, or increased activity of a telomerase relative to normal tissues or to other cancers of the same tissues.

In some embodiments, the tumor cells isolated from subjects diagnosed with cancer can be used to screen test for compounds that alter TERC levels. In some embodiments, the tumor cells can be used to screen test compounds that alter the expressive or activity of PARN or PAPD5. The cancer cells used in the methods can be, e.g., cancer stem cells. Such methods can be used to screen a library of test compounds, e.g., compounds that alter or change expression of protein or RNA of telomere-associated genes (e.g., TERC, PARN, PAPD5/PAPD5).

In some embodiments, agents that decrease the level or activity of TERC (e.g., PANR inhibitors) are used to treat cancer. In some embodiments, these agents are used in combination with other cancer treatments, e.g., chemotherapies, surgery, or radiotherapy.

### Aging

Telomeres shorten over the human life span. In large population based studies, short or shortening telomeres are associated with numerous diseases. Thus, telomeres have an important role in the aging process, and can contribute to various diseases. The role of telomeres as a contributory and interactive factor in aging, disease risks, and protection is described, e.g., in Blackburn, Elizabeth H., Elissa S. Epel, and Jue Lin. “Human telomere biology: A contributory and interactive factor in aging, disease risks, and protection,” Science 350.6265 (2015): 1193-1198, which is incorporated by reference in its entirety.

Telomere attrition is also a major driver of the senescence associated response. In proliferating human cells, progressive telomere erosion ultimately exposes an uncapped free double-stranded chromosome end, triggering a permanent DNA damage response (DDR). The permanent DNA damage response has a profound impact on cell functions. For example, the damage sensor ataxia telangiectasia mutated (ATM) is recruited to uncapped telomeres, leading to the stabilization of tumor suppressor protein 53 (p53) and upregulation of the p53 transcriptional target p21. In turn, p21 prevents cyclin-dependent kinase 2 (CDK2)-mediated inactivation of RB, subsequently preventing entry into the S phase of the cell cycle. Cellular senescence contributes to various age-related diseases, e.g., glaucoma, cataracts, diabetic pancreas, type 2 diabetes mellitus, atherosclerosis, osteoarthritis, inflammation, atherosclerosis, diabetic fat, cancer, pulmonary fibrosis, and liver fibrosis, etc. The permanent DNA damage response and age-related diseases are described, e.g., in Childs, Bennett G., et al. “Cellular senescence in aging and age-related disease: from mechanisms to therapy.” Nature medicine 21.12 (2015): 1424, which is incorporated herein by reference in its entirety.

As used herein, the term “aging” refers to degeneration of organs and tissues over time, in part due to inadequate replicative capacity in stem cells that regenerate tissues over time. Aging may be due to natural disease processes that occur over time, or those that are driven by cell intrinsic or extrinsic pressures that accelerate cellular replication and repair. Such pressures include natural chemical, mechanical, and radiation exposure; biological agents such as bacteria, viruses, fungus, and toxins; autoimmunity, medications, chemotherapy, therapeutic radiation, cellular therapy. As the telomere is an important factor in aging and disease development, the methods described herein can be used for treating, mitigating, or minimizing the risk of, a disorder associated with aging (and/or one or more symptoms of a disorder associated with aging) in a subject. The methods include the step of identifying a subject as having, or being at risk of a disorder associated with aging; and administering a pharmaceutical composition to the subject. In some embodiments, the pharmaceutical composition includes an agent that alters the level or activity of TERC, e.g., increase the level or activity of TERC.

As used herein, the term “disorders associated with aging” or “age-related diseases” refers to disorders that are associated with the ageing process. Exemplary disorders include, e.g., macular degeneration, diabetes mellitus (e.g., type 2 diabetes), osteoarthritis, rheumatoid arthritis, sarcopenia, cardiovascular diseases such as hypertension, atherosclerosis, coronary artery disease, ischemia/reperfusion injury, cancer, premature death, as well as age-related decline in cognitive function, cardiopulmonary function, muscle strength, vision, and hearing.

The disorder associated with aging can also be a degenerative disorder, e.g., a neurodegenerative disorder. Degenerative disorders that can be treated or diagnosed using the methods described herein include those of various organ systems, such as those affecting brain, heart, lung, liver, muscles, bones, blood, gastrointestinal and genito-urinary tracts. In some embodiments, degenerative disorders are those that have shortened telomeres, decreased levels of TERC, and/or decreased levels of telomerase relative to normal tissues. In some embodiments, the degenerative disorder is a neurodegenerative disorder. Exemplary neurodegenerative disorders include Motor Neuron Disease, Creutzfeldt-Jakob disease, Machado-Joseph disease, Spino-cerebellar ataxia, Multiple sclerosis (MS), Parkinson's disease, Alzheimer's disease, Huntington's disease, hearing and balance impairments, ataxias, epilepsy, mood disorders such as schizophrenia, bipolar disorder, and depression, dementia, Pick's Disease, stroke, CNS hypoxia, cerebral senility, and neural injury such as head trauma. Recent studies have shown the association between shorter telomeres and Alzheimer's disease. The relationship between telomere length shortening and Alzheimer's disease is described., e.g., in Zhan, Yigiang, et al. “Telomere length shortening and Alzheimer disease—a Mendelian Randomization Study,” JAMA neurology 72.10 (2015): 1202-1203, which is incorporated by reference in its entirety. In some embodiments, the neurodegenerative disorder is dementia, e.g., Alzheimer's disease.

It has also been determined that there an inverse association between leucocyte telomere length and risk of coronary heart disease. This relationship is described, e.g., in Haycock, Philip C., et al. “Leucocyte telomere length and risk of cardiovascular disease: systematic review and meta-analysis.” (2014): g4227; and Codd, Veryan, et al. “Identification of seven loci affecting mean telomere length and their association with disease.” Nature genetics 45.4 (2013): 422-427; each of which is incorporated by reference in its entirety. Thus, there is strong evidence for a causal role of telomere-length variation in cardiovascular disease (CVD), or coronary artery disease (CAD). In some embodiments, the disorder is a cardiovascular disease (CVD), and/or coronary artery disease (CAD), and the present disclosure provides methods of treating, mitigating, or minimizing the risk of, these disorders. In some cases, the disorder is an atherosclerotic cardiovascular disease.

Furthermore, a meta-analysis of 5759 cases and 6518 controls indicated that shortened telomere length was significantly associated with type 2 diabetes mellitus risk. The relationship between telomere length and type 2 diabetes mellitus is described, e.g., in Zhao, Jinzhao, et al. “Association between telomere length and type 2 diabetes mellitus: a meta-analysis.” PLoS One 8.11 (2013): e79993, which is incorporated by reference in its entirety. In some embodiments, the disorder is a metabolic disorder, e.g., type 2 diabetes mellitus.

In some embodiments, aged cells can be used to screen test compounds that alter the expressive or activity of PARN or PAPD5. The aged cells used in the methods can be, e.g., those with genetic lesions in telomere biology genes, those isolated from elderly subjects, or those that undergo numerous rounds of replication in the lab. Such methods can be used to screen a library of test compounds, e.g., compounds that alter or change expression of protein or RNA of telomere-associated genes (e.g., TERC, PARN, PAPD5/PAPD5). Exemplary methods of screening and screening techniques are described herein.

In some embodiments, agents that increase the level or activity of TERC (e.g., PAPD5/PAPD5 inhibitors) are used to treat age-related degenerative disorders due to natural causes or environmental causes. In some embodiments, these agents are used in combination with other treatments.

### Viral Infections

The hepatitis B virus (HBV) is an enveloped, partially double-stranded D A virus. The compact 3.2 kb HBV genome consists of four overlapping open reading frames (ORF), which encode for the core, polymerase (Pol), envelope and X-proteins. The Pol ORF is the longest and the envelope ORF is located within it, while the X and core ORFs overlap with the Pol ORF. The lifecycle of HBV has two main events: 1) generation of closed circular DNA (cccDNA) from relaxed circular (RC DNA), and 2) reverse transcription of pregenomic RNA (pgRNA) to produce RC DNA. Prior to the infection of host cells, the HBV genome exists within the virion as RC DNA. It has been determined that HBV virions arc able to gain entry into host cells by non-specifically binding to the negatively charged proteoglycans present on the surface of human hepatocytes (Schulze, A., P. Gripon & S. Urban. Hepatology, 46. (2007). 1759-68) and via the specific binding of HBV surface antigens (HBsAg) to the hepatocyte sodium-taurocholate cotransporting polypeptide (NTCP) receptor (Yan, H. et al. J Virol, 87, (2013), 7977-91). Once the virion has entered the cell, the viral cores and the encapsidated RC DNA are transported by host factors, via a nuclear localization signal, into the nucleus through the Impβ/Impα nuclear transport receptors. Inside the nucleus, host DNA repair enzymes convert the RC DNA into cccDNA. cccDNA acts as the template for all viral mRNAs and as such, is responsible for HBV persistence in infected individuals. The transcripts produced from cccDNA are grouped into two categories; Pregenomic RNA (pgRNA) and subgenomic RNA. Subgenomic transcripts encode for the three envelopes (L, M and S) and X proteins, and pgRNA encodes for Pre-Core, Core, and Pol proteins (Quasdorff, M. & U. Protzer. J Viral Hepat, 1 7, (2010), 527-36). Inhibition of HBV gene expression or HBV RNA synthesis leads to the inhibit ion of HBV viral replication and antigens production (Mao, R. et al. PLoS Pathog, 9, (2013), e1003494; Mao, R. et al. J Virol, 85, (2011), 1048-57). For instance, IFN-a was shown to inhibit HBV replication and viral HBsAg production by decreasing the transcription of pgRNA and subgenomic RNA from the HBV covalently closed circular DNA (cccDNA) minichromosome. (Belloni, L. et al. J Clin Invest, 122, (2012), 529-37; Mao, R. et al. J Virol, 85, (2011), 1048-57). All HBV viral mRNAs are capped and polyadenylated and then exported to the cytoplasm for translation. In the cytoplasm, the assembly of new virons is initiated and nascent pgRNA is packaged with viral Pol so that reverse transcription of pgRNA, via a single stranded DNA intermediate, into RC DNA can commence. The mature nucleocapsids containing RC DNA are enveloped with cellular lipids and viral L, M, and S proteins and then the infectious HBV particles are then released by budding at the intracellular membrane (Locarnini, S. Semin Liver Dis, (2005), 25 Suppl 1, 9-1 9). Interestingly, non-infectious particles are also produced that greatly outnumber the infectious virions. These empty, enveloped particles (L, M and S) are referred to as subviral particles. Importantly, since subviral particles share the same envelope proteins and as infectious particles, it has been surmised that they act as decoys to the host immune system and have been used for HBV vaccines. The S, M, and L envelope proteins are expressed from a single ORF that contains three different start codons. All three proteins share a 226aa sequence, the S-domain, at their C-termini. M and L have additional pre-S domains, Pre-S2 and Pre-S2 and Pre-ST, respectively. However, it is the S-domain that has the HBsAg epitope (Lambert, C. & R. Prangc. Virol J, (2007), 4, 45).

The control of viral infection needs a tight surveillance of the host innate immune system which could respond within minutes to hours after infect ion to impact on the initial growth of the virus and limit the development of a chronic and persistent infection. Despite the available current treatments based on IFN and nucleos(t)ide analogues, the Hepatitis B virus (HBV) infection remains a major health problem worldwide which concerns an estimated 350 million chronic carriers who have a higher risk of liver cirrhosis and hepatocellular carcinoma.

The secretion of antiviral cytokines in response to HBV infection by the hepatocytes and/or the intra-hepatic immune cells plays a central role in the viral clearance of infected liver.

However, chronically infected patients only display a weak immune response due to various escape strategies adopted by the virus to counteract the host cell recognition systems and the subsequent antiviral responses.

Many observations showed that several HBV viral proteins could counteract the initial host cellular response by interfering with the viral recognition signaling system and subsequently the interferon (IFN) antiviral activity. Among these, the excessive secretion of HBV empty subviral particles (SVPs, HBsAg) may participate to the maintenance of the immunological tolerant state observed in chronically infected patients (CHB). The persistent exposure to HBsAg and other viral antigens can lead to HBV-specific T-cell deletion o to progressive functional impairment (Kondo et al. Journal of Immunology (1993), 150, 4659 4671; Kondo et al. Journal of Medical Virology (2004), 74, 425 433; Fisicaro et al. Gastroenterology, (2010), 138, 682-93;). Moreover HBsAg has been reported to suppress the function of immune cells such as monocytes, dendritic cells (DCs) and natural killer (NK) cells by direct interaction (Op den Brouw et al. Immunology, (2009b), 1 26, 280-9; Woltman et al. PLoS One, (2011), 6, e15324; Shi et al. J Viral Hepat. (2012). 19, c26-33; Kondo et al. ISRN Gastroenterology, (2013), Article ID 935295).

HBsAg quantification is a significant bio marker for prognosis and treatment response in chronic hepatitis B. However the achievement of HBsAg loss and seroconversion is rarely observed in chronically infected patients but remains the μLtimate goal of therapy. Current therapy such as Nucleos(t)ide analogues are molecules that inhibit HBV DA synthesis but are not directed at reducing HBsAg level. Nucleos(t)ide analogs, even with prolonged therapy, have demonstrated rates of HBsAg clearance comparable to those observed naturally (between−1%-2%) (Janssen et al. Lancet, (2005), 365, 123-9; Marcellin et al. N. Engl. J Med., (2004), 351, 1206-17; Buster et al. Hepatology, (2007), 46, 388-94). Therefore, targeting HBsAg together with HBV DNA levels in CHB patients may significantly improve CHB patient immune reactivation and remission (Wieland, S. F. & F. V. Chisari. J Virol, (2005), 79, 9369-80; Kumar et al. J Virol, (2011), 85, 987-95; Woltman et al. PLoS One, (2011), 6, e15324; Opden Brouw et al. Immunology, (2009b), 126, 280-9).

The compounds of the present disclosure are inhibitors of virion production and inhibitors of production and secretion of surface proteins HBsAg and HBeAg. The compounds reduce effective HBV RNA production at the transcriptional or post-transcriptional levels, such as the result of accelerated viral RNA degradation in the cell. In the alternative, the compounds of the present disclosure inhibit initiation of viral transcription. In sum, the compounds reduce overall levels of HBV RNA, especially HBsAg mRNA, and viral surface proteins. HBsAg may suppress immune reactions against virus or virus infected cells, and high level of HBsAg is thought to be responsible for T cell exhaustion and depletion. Disappearance of HBsAg followed by the emergence of anti-HBsAg antibodies results in a sustained virological response to HBV, which is regarded as a sign of a functional cure.

In some embodiments, the compounds may modulate any of the molecular mechanisms described, for example, in Zhou et al., Antiviral Research 149 (2018) 191-201, which is incorporated herein by reference in its entirety. In some embodiments, the compounds may modulate any of the physiological or molecular mechanisms described, for example, in Mueller et al., Journal of Hepatology 68 (2018) 412-420, which is incorporated herein by reference in its entirety. For example, the compounds of the present disclosure induce HBV RNA degradation (degradation of HBV pgRNA and HBsAg mRNA occurs in the hepatocyte nucleus and requires de novo synthesis of host proteins).

In some embodiments, the compounds of the present disclosure are useful in inhibiting of HBsAg production or secretion, in inhibiting HBV DNA production, and/or in treating or preventing hepatitis B virus (HBV) infection (acute, fulminant, or chronic) in a subject. In some embodiments, the subject is in need of such treatment or prevention (e.g., prior to the administration of the compound of the present disclosure, the subject is diagnosed as having HBV infection by a treating physician).

The compounds are also useful in treating infections caused by viruses in which inhibition of PAPD5/PAPD7 and/or RNA adenylation and/or guanylation is involved in viral RNA production, protein expression and/or replication. In addition to HepB, examples of these viruses include hepatitis A (HepA) and cytomegalovirus (CMV). See Kulsuptrakul et al., A genome-wide CRISPR screen identifies UFMylation and TRAMP-like complexes as host factors required for hepatitis A virus infection, Cell Reports, 2021, 34, 108859; and Kim et al., Viral hijacking of the TENT4-ZCCHC14 complex protects viral RNAs via mixed tailing, Nature structural & molecular biology, 2020, 27, 581-588.

In some embodiments, the compounds of the present disclosure are useful in treating or preventing hepatitis A virus (HAV) infection (acute, fulminant, or chronic) in a subject. In some embodiments, the subject is in need of such treatment or prevention (e.g., prior to the administration of the compound of the present disclosure, the subject is diagnosed as having HAV infection by a treating physician).

In some embodiments, the compounds of the present disclosure are useful in treating or preventing cytomegalovirus (CMV) infection (acute, fulminant, or chronic) in a subject. In some embodiments, the subject is in need of such treatment or prevention (e.g., prior to the administration of the compound of the present disclosure, the subject is diagnosed as having CMV infection by a treating physician).

### Additional Uses

In some embodiments, the compound of the present disclosure modulates RNAs whose transcription, post-transcriptional processing, stability, steady state levels or function are altered due to acquired or genetic defects in one or more of any cellular pathways. In some embodiments, these include non-coding RNAs (ncRNAs) that are members of the small nucleolar RNA (snoRNA), small Cajal body RNA (scaRNA), small nuclear RNA (snRNA), ribosomal RNA (rRNA), Y RNA, transfer RNA (tRNA), microRNA (miRNA), PIWI-interacting RNA (piRNA) or long non-coding RNA (lncRNA) families. The compounds may also by useful for modulating non-coding RNAs in a cell (e.g. scaRNA13, scaRNA8), and concomitantly for preventing and treating the associated disease and conditions. In some embodiments, these also include those ncRNAs affected by any of the molecular mechanisms described, for example, in Lardelli et al, Nature Genetics, 49(3), 2017, 457-464; and in Son et al., 2018, Cell Reports 23, 888-898, including those affected by disruption of PARN or TOE1 deadenylases. As such, the compounds are useful in treating or preventing genetic and other disorders, including neurodevelopmental disorders such as pontocerebellar hypoplasia. Neurodevelopmental disorders are a group of disorders in which the development of the central nervous system is disturbed. This can include developmental brain dysfunction, which can manifest as neuropsychiatric problems or impaired motor function, learning, language or non-verbal communication. In some embodiments, a neurodevelopmental disorder is selected from attention deficit hyperactivity disorder (ADHD), reading disorder (dyslexia), writing disorder (disgraphia), calculation disorder (dyscalculia), expression disorder (ability for oral expression is substantially below the appropriate level for a child's mental age), comprehension disorder (ability for comprehension is markedly below the appropriate level for a child's mental age), mixed receptive-expressive language disorder, speech disorder (dislalia) (inability to use the sounds of speech that are developmentally appropriate), stuttering (disruption of normal fluency and temporal structure of speech), and autism spectrum disorders (persistent difficulties in social communication). In some embodiments, the present disclosure provides a method of treating an acquired or genetic disease or condition associated with alterations in RNA, the method comprising administering to the subject in need thereof a therapeutically effective amount of any one of the compounds described herein, or a pharmaceutically acceptable salt thereof, or a pharmaceutically acceptable composition comprising same. In some embodiments, the RNA comprises ncRNA (e.g., snRNA, scaRNA, snoRNA, rRNA, and miRNA). In some embodiments, the RNA is disrupted by disruption of PARN or TOE1 deadenylase. In some embodiments, the acquired or genetic disease or condition associated with alterations in RNA comprises a neurodevelopmental disorder such as pontocerebellar hypoplasia.

Because the compounds are PAPD5 inhibitors, and because these affect TERC, telomerase, telomere maintenance and stem cell self-renewal, the compounds are useful in modulating ex vivo expansion of stem cells, and also useful for allograft exhaustion, in hematopoietic or other tissues. For example, PAPD5 inhibitors may be useful for the ex vivo expansion of hematopoietic stem cells as described in Fares, et al, 2015, Science 345, 1590-1512, and Boitano, et al, 2010 329, 1345-1348, both of which are incorporated by reference herein in their entireties.

### CRISPR/Cas9 (CRISPR-Associated 9)

Genome engineering and genetic modulation by the control of individual gene expression can be used in therapeutics as well. CRISPR (clustered regularly interspaced short palindromic repeats) is a family of DNA sequences found within the genomes of prokaryotic organisms such as bacteria and archaea. CRISPR/Cas RNA-guided genome targeting and gene regulation in mammalian cells (e.g., using modified bacterial CRISPR/Cas components) can be used to inhibit the expression and/or activity of genes (e.g., PAPD5).

In some embodiments, a catalytically silent Cas-9 mutant (a null nuclease) can be tethered to specified gene promoter regions and has the effect of reducing expression of those genes. In some embodiments, the Cas-9 mutant is linked to a transcription factor.

In some embodiments, the CRISPR/Cas9 genome targeting can create biallelic null mutations, thus inhibit the expression and the activity of a gene (e.g., PAPD5). Thus, in some embodiments, the PAPD5 inhibitor can be a vector that encode guide RNAs (gRNAs) that target PAPD5 for CRISPR/Cas9, wherein CRISPR/Cas9 creates null mutations in PAPD5, thereby decreasing the level and activity of PAPD5. In some embodiments, the PAPD5 inhibitor includes the CRISPR/Cas9 system and the guide RNAs. In some embodiments, the guide RNA can have the following sequences:

(SEQ ID NO: 2)
CCUCUUGUUGCUGCUGCCCG;
(SEQ ID NO: 3)
CGGAGCGAUACAUGCCGGCC;
or
(SEQ ID NO: 4)
CCUCUUGUUGCUGCUGCCCG.

The CRISPR/Cas9 targeting can be used in the various methods as described herein, for example, modulating telomerase RNA component, screening, diagnosing, treating or preventing a disease or condition selected from: a disorder associated with telomere or telomerase dysfunction, a disorder associated with aging, a pre-leukemic or pre-cancerous condition, a viral infection (e.g., an HBV infection), a neurodevelopmental disorder, and an acquired or genetic disease or condition associated with alterations in RNA, etc.

### Diagnosing a Subject in Need of Treatment

The present specification provides methods of diagnosing a subject in need of treatment (e.g., as having any one of telomere diseases described herein). As an example, if the level or activity of TERC, PARN, and/or PAPD5 in a subject is comparable to the level or activity of TERC, PARN, and/or PAPD5 in a subject having the telomere disease and, optionally, the subject has one or more symptoms associated with telomere disease (e.g., aplastic anemia, pulmonary fibrosis, hepatic cirrhosis), then the subject can be diagnosed as having or being at risk of developing a telomere disease.

In some embodiments, if the level or activity of TERC, PARN, and/or PAPD5 in a subject is comparable to the level or activity of TERC, PARN, and/or PAPD5 in a control subject who does not have a telomere disease, then the subject can be diagnosed as not having telomere disease or not being at risk of developing a telomere disease.

In some embodiments, the subject is determined to have or being at risk of developing a telomere disease if there is a mutation at PARN. The mutation can be a missense mutation, deletion or truncation mutation, omission of single or groups of nucleotides encoding one or several amino acids, non-coding mutation such as promoter, enhancer, or splicing mutation, or other mutations. (See, e.g., Nagpal, et al, Cell Stem Cell, 2020. The mutation can be a deletion containing part of PARN gene or the entire PARN gene. The mutation can also be a mutation at position 7 and/or 87 of PARN, e.g., the amino acid residue at position 7 is not asparagine, and/or the amino acid residue at position 87 of PARN is not serine. For example, the mutation can be a missense variant c.19A>C, resulting in a substitution of a highly conserved amino acid p.Asn7His. In some cases, the mutation is a missense mutation c.260C>T, encoding the substitution of a highly conserved amino acid, p.Ser87Leu. In some embodiments, the subject is determined to have or be at risk of developing a telomere disease if there is a mutation in DKC1. The mutation can be a missense mutation, deletion or truncation mutation, omission of single or groups of nucleotides encoding one or several amino acids, non-coding mutation such as promoter, enhancer, or splicing mutation, or other mutations. (See, e.g., Fok, et al, Blood, 2019; and Nagpal, et al, Cell Stem Cell, 2020). In some embodiments, the subject is determined to have or be at risk of developing a telomere disease if there is a mutation in any factor that regulates TERC, including NOP10, NHP2, NAF1, GAR1, TCAB1/WRAP53, ZCCHC8, and TERC itself. The mutation can be a missense mutation, deletion or truncation mutation of whole or part of the gene, omission of single or groups of amino acids. In some embodiments the subject is determined to have or be at risk of developing a telomere disease if there is a mutation in any factor that regulates telomere biology, such as TERT, TINF2, ACD/TPP1, STN1. CTC1, or POT1. The mutation can be a missense mutation, deletion or truncation mutation, omission of single or groups of nucleotides encoding one or several amino acids, non-coding mutation such as promoter, enhancer, or splicing mutation, or other mutations.

In some embodiments, a subject has no overt signs or symptoms of a telomere disease, but the level or activity of TERC, PARN or PAPD5 may be associated with the presence of a telomeres disease, then the subject has an increased risk of developing telomere disease. In some embodiments, once it has been determined that a person has telomere disease, or has an increased risk of developing telomere disease, then a treatment, e.g., with a small molecule (e.g., a PAPD5 inhibitor) or a nucleic acid encoded by a construct, as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of PAPD5 protein, e.g., a control reference level that represents a normal level of PAPD5 protein, e.g., a level in an unaffected subject or a subject who is not at risk of developing a disease described herein, and/or a disease reference that represents a level of the proteins associated with conditions associated with telomere disease, e.g., a level in a subject having telomere disease (e.g., pulmonary fibrosis, hepatic cirrhosis or aplastic anemia). In another embodiment, the reference comprises a predetermined value for a meaningful level of PARN protein, e.g., a control reference level that represents a normal level of PARN protein, e.g., a level in an unaffected subject or a subject who is not at risk of developing a disease described herein, and/or a disease reference that represents a level of the proteins associated with conditions associated with telomere disease, e.g., a level in a subject having telomere disease (e.g., pulmonary fibrosis, hepatic cirrhosis or aplastic anemia).

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have a disorder described herein. In some embodiments, it may be desirable that the control subject is deficient in PARN gene (e.g., Dyskeratosis Congenita), and in other embodiments, it may be desirable that a control subject has cancer. In some cases, it may be desirable that the control subject has high telomerase activity, and in other cases it may be desirable that a control subject does not have substantial telomerase activity.

In some embodiments, the level of TERC or PARN in a subject being less than or equal to a reference level of TERC or PARN is indicative of a clinical status (e.g., indicative of a disorder as described herein, e.g., telomere disease). In some embodiments, the activity of TERC or PARN in a subject being greater than or equal to the reference activity level of TERC or PARN is indicative of the absence of disease.

The predetermined value can depend upon the particular population of subjects (e.g., human subjects or animal models) selected. For example, an apparently healthy population will have a different ‘normal’ range of levels of TERC than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art. In characterizing likelihood, or risk, numerous predetermined values can be established.

In some embodiments, the methods described in this disclosure involves identifying a subject as having, being at risk of developing, or suspected of having a disorder associated with telomerase dysfunction. The methods include determining the level or activity of TERC, PARN, or PAPD5 in a cell from the subject; comparing the level or activity of TERC, PARN, or PAPD5 to the reference level or reference activity of TERC, PARN, or PAPD5; and identifying the subject as having, being at risk of developing, or suspected of having a disorder associated with telomerase dysfunction if the level or activity of TERC, PARN, or PAPD5 is significantly different from the reference level or activity of TERC, PARN, or PAPD5. In some embodiments, the reference level or activity of TERC, PARN, or PAPD5 are determined by cells obtained from subjects without disorders associated with telomerase dysfunction.

The level or activity of TERC, PARN, or PAPD5 can be determined in various types of cells from a subject. The methods can include obtaining cells from a subject, and transforming these cells to induced pluripotent stem cells (I-IPS) cells, and these iPS cells can be used to determine the level or activity of TERC, PARN, or PAPD5. These cells can be, e.g., primary human cells or tumor cells. Pluripotent stem cells (I-IPS) cells can be generated from somatic cells by methods known in the art (e.g., somatic cells may be genetically reprogrammed to an embryonic stem cell-like state by being forced to express genes and factors important for maintaining the defining properties of embryonic stem cells). In some embodiments, the methods of diagnosing a subject include analyzing blood sample of the subject, or a sample of hair, urine, saliva, or feces of the subject (e.g., a subject may be diagnosed without any cell culture surgically obtained from the subject).

The subject may be one having a mutation at PARN, e.g., a deletion containing part of PARN gene or the entire PARN gene. For example, the mutation may be one wherein the amino acid residue at position 7 of PARN is not asparagine or serine. For example, the subject can have a missense variant c.19A>C, resulting in a substitution of a highly conserved amino acid p.Asn7His. The subject can have a missense mutation c.260C>T, encoding the substitution of a highly conserved amino acid, p.Ser87Leu.

### Induced Pluripotent Stem Cells

Induced pluripotent stem cells (I-IPSC or iPS), are somatic cells (e.g., derived from patient skin or other cell) that have been genetically reprogrammed to an embryonic stem cell-like state by being forced to express genes and factors important for maintaining the defining properties of embryonic stem cells. These cells can be generated by methods known in the art.

It is known that mouse iPSCs demonstrate important characteristics of pluripotent stem cells, including expressing stem cell markers, forming tumors containing cells from all three germ layers, and being able to contribute to many different tissues, when injected into mouse embryos at a very early stage in development.

Human iPSCs also express stem cell markers and are capable of generating cells characteristic of all three germ layers. iPSCs can be generated from human fibroblasts and are already useful tools for drug development and modeling of diseases. Viruses are currently used to introduce the reprogramming factors into adult cells (e.g., lentiviral vectors disclosed herein), and this process can be carefully controlled and tested in cultured, isolated cells first to then treat cells (e.g., by contacting with a test compound) to express altered markers, e.g., iPSCs from tumor cells can be manipulated to differentiate or iPSCs from cardiomyocytes can be manipulated to de-differentiate.

The iPSC manipulation strategy can be applied to any cells obtained from a subject to test whether the compound can alter the level or activity of TERC, PARN, or PAPD5. The cells are contacted with test compounds (e.g., small molecules). In some embodiments, these iPSC cells can be used for screening compounds that modulate TERC. In some embodiments, the iPSC cells can be converted from patient skin fibroblasts.

### Cell Expansion

The present disclosure provides methods of expanding a cell population by culturing one or more cells in the presence of compounds as disclosed herein (e.g., compounds of Formulae (I), (II), (III), or (IV)). In some embodiments, cell expansion can involve contacting the cells with an effective amount of compound of the present disclosure (e.g., PAPD5 inhibitors of Formulae (I), (II), (III), or (IV)). The PAPD5 inhibitors can decrease the level and activity of PAPD5, thereby increasing or maintaining the length of the telomere. Telomerase activity and telomere length maintenance are related to cell expansion capability. As the cell divides, the telomere length gradually shortens, eventually leading to senescence of cells. Based on the telomere theory, aging in cells is irreversible. Programmed cell cycle arrest happens in response to the telomerase activity and the total number of cell divisions cannot exceed a particular limit termed the Hayflick limit. It has been determined that maintaining telomere length during cell replication is important for cell expansion (e.g., stem cell expansion). The present disclosure provides methods of promoting cell expansion, and methods of inhibiting, slowing, or preventing cell aging.

In some embodiments, the cell is a stem cell. Stem cells can include, but are not limited to, for example, pluripotent stem cells, embryonic stem cells, hematopoietic stem cells, adipose derived stem cells, mesenchymal stem cells, umbilical cord blood stem cells, placentally derived stem cells, exfoliated tooth derived stem cells, hair follicle stem cells, or neural stem cells. In some embodiments, the cell is a peripheral blood mononuclear (PBMC) cell.

The cells can be derived from the subject with a disease or condition associated with any disorder described herein, e.g., cancer, a telomere or telomerase dysfunction, a disorder associated with aging, a pre-leukemic or pre-cancerous condition, and a neurodevelopment disorder. The cells can be isolated and derived, for example, from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, or mesentery tissue.

The cells can be isolated from any mammalian organism, e.g., human, mouse, rats, dogs, or cats, by any means know to one of ordinary skill in the art. One skilled in the art can isolate embryonic or adult tissues and obtain various cells (e.g., stem cells).

The expanded cell population can be further enriched by using appropriate cell markers. For example, stem cells can be enriched by using specific stem cell markers, e.g., FLK-1, AC133, CD34, c-kit, CXCR-4, Oct-4, Rex-1, CD9, CD13, CD29, CD34, CD44, CD166, CD90, CD105, SH-3, SH-4, TRA-1-60, TRA-1-81, SSEA-4, and Sox-2. One skilled in the art can enrich a specific cell population by using antibodies known in the art against any of these cell markers. In some embodiments, expanded stem cells can be purified based on desired stem cell markers by fluorescence activated cell sorting (FACS), or magnet activated cell sorting (MACS).

The cells (e.g., stem cells) can be cultured and expanded in suitable growth media.

Commonly used growth media include, but are not limited to, Iscove's modified Dulbecco's Media (IMDM) medium, McCoy's 5A medium, Dulbecco's Modified Eagle medium (DMEM), KnockOut™ Dulbecco's Modified Eagle medium (KO-DMEM), Dulbecco's Modified Eagle Medium/Nutrient Mixture F-12 (DMEM/F12), Roswell Park Memorial Institute (RPMI) medium, minimum essential medium alpha medium (α-MEM), F-12K nutrient mixture medium (Kaighn's modification, F-12K), X-vivo™ 20 medium, Stemline™ medium, StemSpan™ CC100 medium, StemSpan™ H2000 medium, MCDB 131 Medium, Basal Media Eagle (BME), Glasgow Minimum Essential medium (GMEM), Modified Eagle Medium (MEM), Opti-MEM I Reduced Serum medium, Waymouth's MB 752/1 Medium, Williams' Medium E, NCTC-109 Medium, neuroplasma medium, BGJb Medium, Brinster's BMOC-3 Medium, Connaught Medical Research Laboratories (CMRL) Medium, CO₂-Independent Medium, and Leibovitz's L-15 medium.

The compounds of the present disclosure (e.g., compounds of Formulae (I), (II), or (III)) can be used to expand various cell population, e.g., by adding the compound in cell culture media in a tube or plate. The concentration of the compound can be determined by, but limited to, the time of cell expansion. For example, the cells can be in culture with high concentration of the compound for a short period of time, e.g., at least or about 1 day, 2 days, 3 days, 4 days, or 5 days. In some embodiments, the cells can be cultured with a low concentration of the compound for a long period of time, e.g., at least or about 3 days, 4 days, 5 days, 6 days, 1 week, 2 weeks, 3 weeks, or 4 weeks.

In some embodiments, growth factors are also added to the growth medium to expand cells. Examples of suitable growth factors include, but are not limited to, thrombopoietin, stem cell factor, IL-1, IL-3, IL-7, flt-3 ligand, G-CSF, GM-CSF, Epo, FGF-1, FGF-2, FGF-4, FGF-20, IGF, EGF, NGF, LIF, PDGF, bone morphogenic proteins, activin-A, VEGF, forskolin, and glucocorticords. Further, one skilled in the art, using methods known in the art, can add a feeder layer to the culture medium. A feeder layer can include cells such as, placental tissue or cells thereof.

The methods described herein can also be used to produce and expand Chimeric Antigen Receptor (CAR) T-Cells. CAR-T cell therapies involve genetic modification of patient's autologous T-cells to express a CAR specific for a tumor antigen, following by ex vivo cell expansion and re-infusion back to the patient. PBMCs can be collected from a patient and cultured in the presence of the compounds as described herein (e.g., compounds of Formulae (I), (II), (III), or (IV)), with appropriate media (e.g., complete media containing 30 U/mL interleukin-2 and anti-CD3/CD28 beads). The cells can be expanded for about 3 to 14 days (e.g., about 3 to 7 days). Subsets of T cells can be sorted by FACS. Gating strategies for cell sorting can exclude other blood cells, including granulocytes, monocytes, natural killer cells, dendritic cells, and B cells. Primary T cells are then transduced by incubating cells with the CAR-expressing lentiviral vector in the culture media. In some embodiments, the culture media can be supplemented with the compounds as described herein. The transduced cells are then cultured for at least a few days (e.g., 3 days) before being used in CAR-T cell therapies.

In some embodiments, the present disclosure provides a method of expanding a cell, the method comprising culturing the cell in the presence of an effective amount of a compound as described herein (e.g., a compound of Formulae (I), (II), (III), or (IV)), or a pharmaceutically acceptable salt thereof.

In some embodiments, the cell is selected from the group consisting of: stem cell, pluripotent stem cell, hematopoietic stem cell, and embryonic stem cell.

In some embodiments, the cell is a pluripotent stem cell.

In some embodiments, the cell is a hematopoietic stem cell.

In some embodiments, the cell is an embryonic stem cell.

In some embodiments, the cell is collected from a subject with a disease or condition selected from the group consisting of a disorder associated with telomere or telomerase dysfunction, a disorder associated with aging, a pre-leukemic or pre-cancerous condition, and a neurodevelopment disorder.

In some embodiments, the method further comprises culturing the cell with a feeder layer in a medium.

In some embodiments, the cell has at least one stem cell marker selected from the group consisting of FLK-1, AC133, CD34, c-kit, CXCR-4, Oct-4, Rex-1, CD9, CD13, CD29, CD34, CD44, CD166, CD90, CD105, SH-3, SH-4, TRA-1-60, TRA-1-81, SSEA-4, and Sox-2.

In some embodiments, the stem cell marker is CD34.

In some embodiments, the method further comprising enriching stem cells by isolating CD34+ cells.

In some embodiments, the subject is a mammal.

In some embodiments, the subject is a human.

In some embodiments, the method comprises culturing the cell in a medium selected from the group consisting of Iscove's modified Dulbecco's Media (IMDM) medium, Dulbecco's Modified Eagle Medium (DMEM), Roswell Park Memorial Institute (RPMI) medium, minimum essential medium alpha medium (α-MEM), Basal Media Eagle (BME) medium, Glasgow Minimum Essential Medium (GMEM), Modified Eagle Medium (MEM), Opti-MEM I Reduced Serum medium, neuroplasma medium, CO₂-independent medium, and Leibovitz's L-15 medium.

In some embodiments, the cell is a Chimeric Antigen Receptor (CAR) T-Cell.

In some embodiments, the cell is a lymphocyte.

In some embodiments, the cell is a T cell, an engineered T cell, or a natural killer cell (NK).

### Pharmaceutical Compositions and Formulations

The present application also provides pharmaceutical compositions comprising an effective amount of any one of the compounds disclosed herein, or a pharmaceutically acceptable salt thereof, and a pharmaceutically acceptable carrier. The pharmaceutical composition can also comprise at least one of any one of the additional therapeutic agents described herein. In certain embodiments, the application also provides pharmaceutical compositions and dosage forms comprising any one the additional therapeutic agents described herein (e.g., in a kit). The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that can be used in the pharmaceutical compositions of the present application include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms can contain any one of the compounds and therapeutic agents described herein in the range of 0.005% to 100% with the balance made up from the suitable pharmaceutically acceptable excipients. The contemplated compositions can contain 0.001%-100% of any one of the compounds and therapeutic agents provided herein, in one embodiment 0.1-95%, in another embodiment 75-85%, in a further embodiment 20-80%, wherein the balance can be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein can conveniently be presented in a unit dosage form, e.g., tablets, capsules (e.g., hard or soft gelatin capsules), sustained release capsules, and in liposomes, and can be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration can be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which can beneficially increase the rate of compound absorption. In the case of tablets for oral use, carriers that are commonly used include lactose, sucrose, glucose, mannitol, and silicic acid and starches. Other acceptable excipients can include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents can be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which can contain antioxidants, buffers, bacteriostats and solutes which render the formulation isotonic with the blood of the intended recipient; and aqueous and non-aqueous sterile suspensions which can include suspending agents and thickening agents. The formulations can be presented in unit-dose or multi-dose containers, for example, sealed ampules and vials, and can be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions can be prepared from sterile powders, granules and tablets. The injection solutions can be in the form, for example, of a sterile injectable aqueous or oleaginous suspension. This suspension can be formulated according to techniques known in the art using suitable dispersing or wetting agents and suspending agents. The sterile injectable preparation can also be a sterile injectable solution or suspension in a non-toxic parenterally-acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium. For this purpose, any bland fixed oil can be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant.

The pharmaceutical compositions of the present application can be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include cocoa butter, beeswax, and polyethylene glycols.

The pharmaceutical compositions of the present application can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application can be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polydimethylsiloxane, polycaprolactone, polyethylene glycol, polylactic acid, ethylene vinyl acetate, and mixtures thereof. The coatings can optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a therapeutic compound is present in an effective amount (e.g., a therapeutically effective amount).

Effective doses can vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a therapeutic compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a therapeutic compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month). The compounds and compositions described herein can be administered to the subject in any order. A first therapeutic agent, such as a compound of any one of the Formulae disclosed herein, can be administered prior to or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before or after), or concomitantly with the administration of a second therapeutic agent, such as an anti-cancer therapy described herein, to a subject in need of treatment. Thus, the compound of any one of the Formulae disclosed herein, or a composition containing the compound, can be administered separately, sequentially or simultaneously with the second therapeutic agent, such as a chemotherapeutic agent described herein. When the compound of any one of the Formulae disclosed herein, or a pharmaceutically acceptable salt thereof, and a second or third therapeutic agent are administered to the subject simultaneously, the therapeutic agents can be administered in a single dosage form (e.g., tablet, capsule, or a solution for injection or infusion).

### Combination Therapies

In some embodiments, the compounds described here may be administered to a subject in any combination with treatments for telomere diseases that are known in the art. The combination treatment may be administered to the subject either consecutively or concomitantly with the compound of any one of the Formulae disclosed herein. When combination treatment comprises an alternative therapeutic agent, the therapeutic agent may be administered to the subject in any one of the pharmaceutical compositions described herein.

In some embodiments, the compounds of the present disclosure may be used in combination with a therapeutic agent that is useful in treating a telomere disease (e.g., a therapeutic agent that modulates the level or activity of TERC). In some embodiments, the agent useful in treating a telomere disease is a nucleic acid comprising a nucleotide sequence that encodes PARN. The agent can also be an anti-PARN antibody or anti-PARN antibody fragment. In some embodiments, the agent is an antisense molecule or a small interfering nucleic acid which is specific for a nucleic acid encoding PARN. In some embodiments, the agent is a nucleic acid comprising a nucleotide sequence that encodes PAPD5. The agent can also be an anti-PAPD5 antibody or anti-PAPD5 antibody fragment. In some embodiments, the agent is an antisense molecule or a small interfering nucleic acid which is specific for a nucleic acid encoding PAPD5. The antisense molecule described herein can be an oligonucleotide. In some cases, the agent binds to PARN or PAPD5.

In some embodiments, the therapeutic agent that is useful in treating a telomere disease is selected from adenosine analogues, aminoglycosides, and purine nucleotides, etc. In some cases, the aminoglycoside can be a member of the neomycin and kanamycin families. The aminoglycoside can be, for example, kanamycin B sulfate, pramycin sulfate, spectinomycin dihydrochloride pentahydrate, ribostamycin sulfate, sisomicin sulfate, amikacin disulfide, dihydrostreptomycin sesquisulfate, hygromycin B, netilmicin sulfate, paromomycin sulfate, kasugamycin, neomycin, gentamicin, tobramycin sulfate, streptomycin sulfate, or neomycin B, or derivatives thereof.

In some embodiments, the therapeutic agent that is useful in treating a telomere disease a nucleoside analogue, e.g., an adenosine analogue, 8-chloroadenosine (8-Cl-Ado) and 8-aminoadenosine (8-amino-Ado), or the triphosphate derivative thereof, synthetic nucleoside analogue bearing a fluoroglucopyranosyl sugar moiety, benzoyl-modified cytosine or adenine, adenosine- and cytosine-based glucopyranosyl nucleoside analogue, or glucopyranosyl analogue bearing uracil, 5-fluorouracil or thymine, etc.

Adenosine analogues, aminoglycosides, and purine nucleotides are known in the art, and they are described, e.g., in Kim, Kyumin, et al. “Exosome Cofactors Connect Transcription Termination to RNA Processing by Guiding Terminated Transcripts to the Appropriate Exonuclease within the Nuclear Exosome.” Journal of Biological Chemistry (2016): jbc-M116; Chen, Lisa S., et al. “Chain termination and inhibition of mammalian poly (A) polymerase by modified ATP analogues.” Biochemical pharmacology 79.5 (2010): 669-677; Ren, Yan-Guo, et al. “Inhibition of Klenow DNA polymerase and poly (A)-specific ribonuclease by aminoglycosides.” Rna 8.11 (2002): 1393-1400; Thuresson, Ann-Charlotte, Leif A. Kirsebom, and Anders Virtanen. “Inhibition of poly (A) polymerase by aminoglycosides.” Biochimie 89.10 (2007): 1221-1227; AA Balatsos, N., et al. “Modulation of poly (A)-specific ribonuclease (PARN): current knowledge and perspectives.” Current medicinal chemistry 19.28 (2012): 4838-4849; Balatsos, Nikolaos A A, Dimitrios Anastasakis, and Constantinos Stathopoulos. “Inhibition of human poly (A)-specific ribonuclease (PARN) by purine nucleotides: kinetic analysis.” Journal of enzyme inhibition and medicinal chemistry 24.2 (2009): 516-523; Balatsos, Nikolaos A A, et al. “Competitive inhibition of human poly (A)-specific ribonuclease (PARN) by synthetic fluoro-pyranosyl nucleosides.” Biochemistry 48.26 (2009): 6044-6051; and Balatsos, Nikolaos, et al. “Kinetic and in silico analysis of the slow-binding inhibition of human poly (A)-specific ribonuclease (PARN) by novel nucleoside analogues.” Biochimie 94.1 (2012): 214-221; each of which is incorporated herein by reference in its entirety. Numerous therapeutic agents that can modulate the level or activity of PARN and/or PAPD5 are described, e.g., in WO 2017/066796, which is incorporated herein by reference in its entirety.

In some embodiments, the compounds of the present disclosure are used in combination with an anti-cancer therapy. In some embodiments, the anti-cancer therapy is selected from the group consisting of surgery, radiation therapy, chemotherapy, gene therapy, DNA therapy, viral therapy, RNA therapy, adjuvant therapy, and immunotherapy. In some embodiments, the anti-cancer therapy is selected from the group consisting of a platinum agent, mitomycin C, a poly (ADP-ribose) polymerase (PARP) inhibitor, a radioisotope, a vinca alkaloid, an antitumor alkylating agent, a monoclonal antibody and an antimetabolite. In some embodiments, the anti-cancer therapy is an ataxia telangiectasia mutated (ATM) kinase inhibitor. Suitable examples of platinum agents include cisplatin, carboplatin, oxaliplatin, satraplatin, picoplatin, nedaplatin, triplatin, and lipoplatin. Suitable examples of cytotoxic radioisotopes include ⁶⁷Cu, ⁶⁷Ga, ⁹⁰Y, ¹³¹I, ¹⁷⁷Lu, ¹⁸⁶Re, ¹⁸⁸Re, α-Particle emitter, ²¹¹At, ²¹³Bi, ²²⁵Ac, Auger-electron emitter, ¹²⁵I, ²¹²Pb, and ¹¹¹In. Suitable examples of antitumor alkylating agents include nitrogen mustards, cyclophosphamide, mechlorethamine or mustine (HN2), uramustine or uracil mustard, melphalan, chlorambucil, ifosfamide, bendamustine, nitrosoureas, carmustine, lomustine, streptozocin, alkyl sulfonates, busulfan, thiotepa, procarbazine, altretamine, triazenes, dacarbazine, mitozolomide, and temozolomide. Suitable examples of anti-cancer monoclonal antibodies include to necitumumab, dinutuximab, nivolumab, blinatumomab, pembrolizumab, ramucirumab, obinutuzumab, adotrastuzumab emtansine, pertuzumab, brentuximab, ipilimumab, ofatumumab, catumaxomab, bevacizumab, cetuximab, tositumomab-I¹³¹, ibritumomab tiuxetan, alemtuzumab, gemtuzumab ozogamicin, trastuzumab, and rituximab. Suitable examples of vinca alkaloids include vinblastine, vincristine, vindesine, vinorelbine, desoxyvincaminol, vincaminol, vinburnine, vincamajine, vineridine, vinburnine, and vinpocetine. Suitable examples of antimetabolites include fluorouracil, cladribine, capecitabine, mercaptopurine, pemetrexed, fludarabine, gemcitabine, hydroxyurea, methotrexate, nelarbine, clofarabine, cytarabine, decitabine, pralatrexate, floxuridine, and thioguanine.

### Kits

The present disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit can optionally include directions to perform a test to determine that a subject is in need of treatment with a compound of any one of Formulae (I)-(IV) as described herein, and/or any of the reagents and device(s) to perform such tests. The kit can also optionally include an additional therapeutic agent (e.g., a nucleic acid comprising a nucleotide sequence that encodes PARN or PAPD5).

### Definitions

As used herein, the term “about” means “approximately” (e.g., plus or minus approximately 10% of the indicated value).

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tert-butoxycarbonyl), and the like.

As used herein, the term “Cₙ₋ₘ alkylcarbonyl” refers to a group of formula —C(O)— alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tert-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)— alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom as a divalent substituent, forming a carbonyl group when attached to a carbon (e.g., C═O), or attached to a heteroatom forming a sulfoxide or sulfone group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the PAPD5 with a compound of the invention includes the administration of a compound of the present invention to an individual or patient, such as a human, having PAPD5, as well as, for example, introducing a compound of the invention into a sample containing a cellular or purified preparation containing the PAPD5.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Example 1A—Inhibition of Recombinant PAPD5

Recombinant PAPD5 (rPAPD5) was purified for in vitro assays. An in vitro RNA polyadenylation assay using recombinant PAPD5, ATP and an oligonucleotide substrate was performed. For gel-based detection of substrate extension, the polyadenylation reactions were performed in a buffer containing 25 mM Tris-HCl (pH7.4), 50 mM KCl, 5 mM MgCl₂, and 50 mM ATP). 1 pmol of 5′-FAM-labeled RNA oligo (CUGC)5 (Integrated DNA Technologies) and 2.5 pmol of purified rPAPD5 were added per 10 ml of the reaction mix followed by incubation at room temperature for 1 hr. Test compounds were added to a final concentration ranging from 0.1-100 μM from a 10 mM stock in dimethylsufoxide (DMSO). Reactions were incubated at room temperature for 1 hour and stopped using formamide loading buffer (10 mM EDTA and 83.3% formamide) and resolved using denaturing polyacrylamide gels (15% Criterion TBE-Urea Polyacrylamide Gel, 26 well, 15 ml, Bio-Rad, 3450093). Gels were imaged using a FLA9000 imager (GE Healthcare). RNA oligo-extension inhibition for certain tested compounds is shown in the corresponding Figures. Referring to those figures, cmpd. 1 is a compound having the formula:

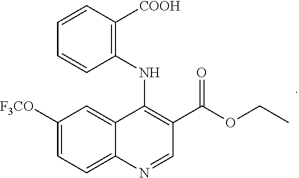 [SMILES: CCOC(=O)c1cnc2ccc(OC(F)(F)F)cc2c1Nc1ccccc1C(=O)O]

### Example 1B

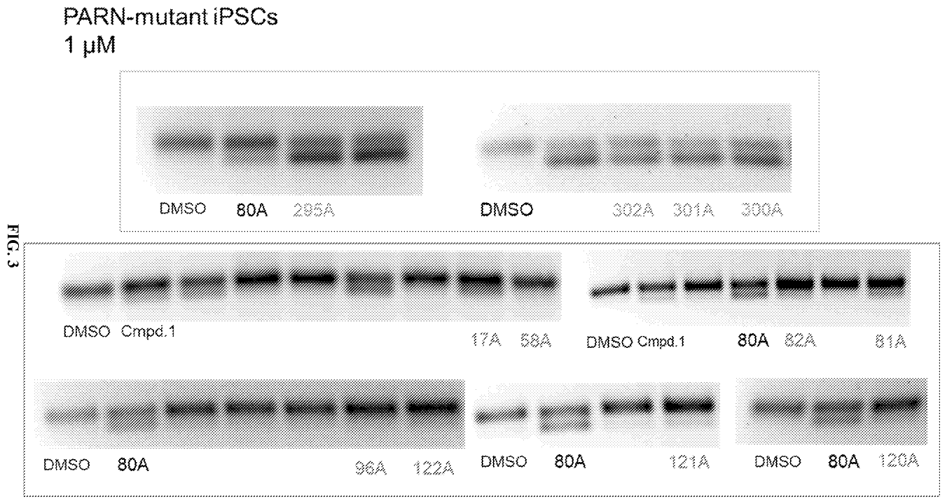

FIG. 3 shows TERC 3′ end processing—Rapid Amplification of cDNA Ends (RACE) for exemplified compounds 295A, 302A, 301A, and 300A. The data is also shown for compounds 17A, 58A, 82A, 81A, 96A, 122A, 121A, and 120A, the structures of which are shown below.

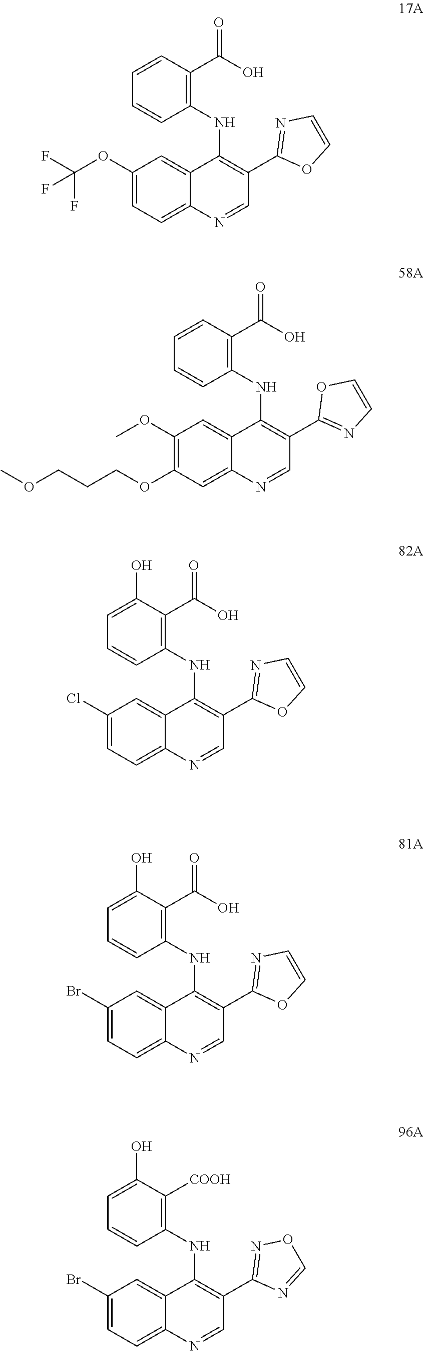 [SMILES: O=C(O)c1ccccc1Nc1c(-c2ncco2)cnc2ccc(OC(F)(F)F)cc12 ; COCCCOc1cc2ncc(-c3ncco3)c(Nc3ccccc3C(=O)O)c2cc1OC ; O=C(O)c1c(O)cccc1Nc1c(-c2ncco2)cnc2ccc(Cl)cc12 ; O=C(O)c1c(O)cccc1Nc1c(-c2ncco2)cnc2ccc(Br)cc12 ; O=C(O)c1c(O)cccc1Nc1c(-c2ncon2)cnc2ccc(Br)cc12]

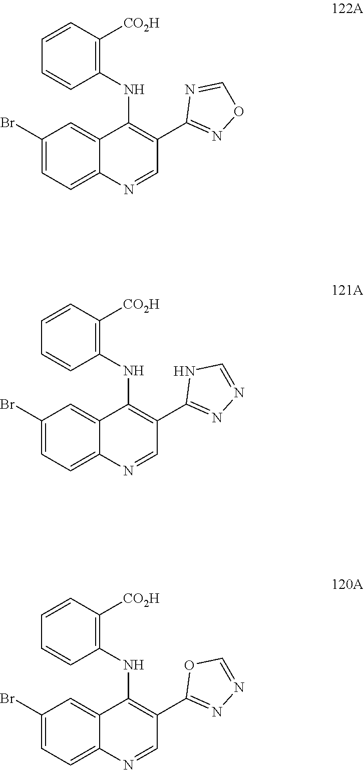 [SMILES: O=C(O)c1ccccc1Nc1c(-c2ncon2)cnc2ccc(Br)cc12 ; O=C(O)c1ccccc1Nc1c(-c2nnc[nH]2)cnc2ccc(Br)cc12 ; O=C(O)c1ccccc1Nc1c(-c2nnco2)cnc2ccc(Br)cc12]

### Example 1C

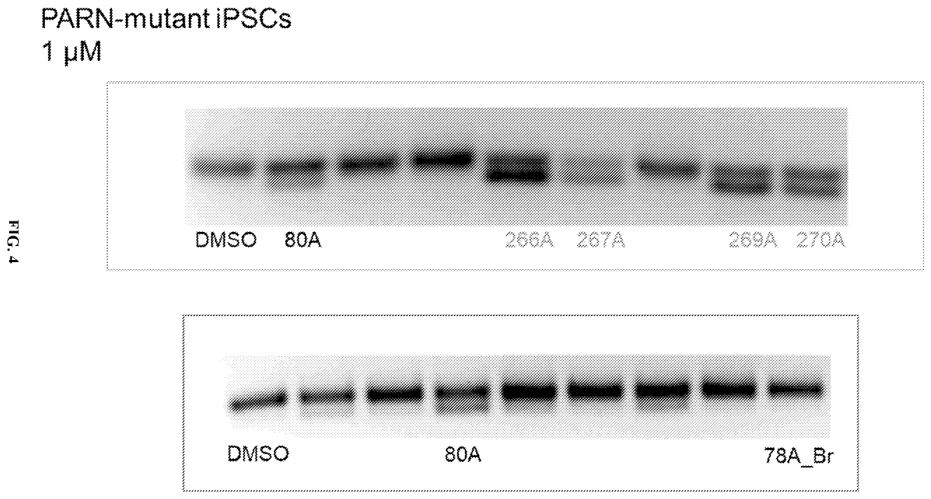

FIG. 4 shows TERC 3′ end processing—Rapid Amplification of cDNA Ends (RACE) for exemplified compounds 266A, 267A, 269A, and 270A. The data is also shown for compound 78A_Br, the structure of which are shown below:

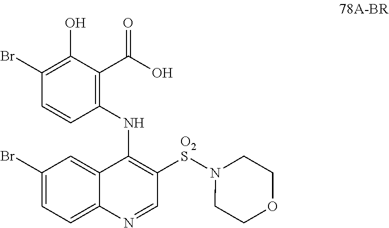 [SMILES: O=C(O)c1c(Nc2c(S(=O)(=O)N3CCOCC3)cnc3ccc(Br)cc23)ccc(Br)c1O]

### Example 1D

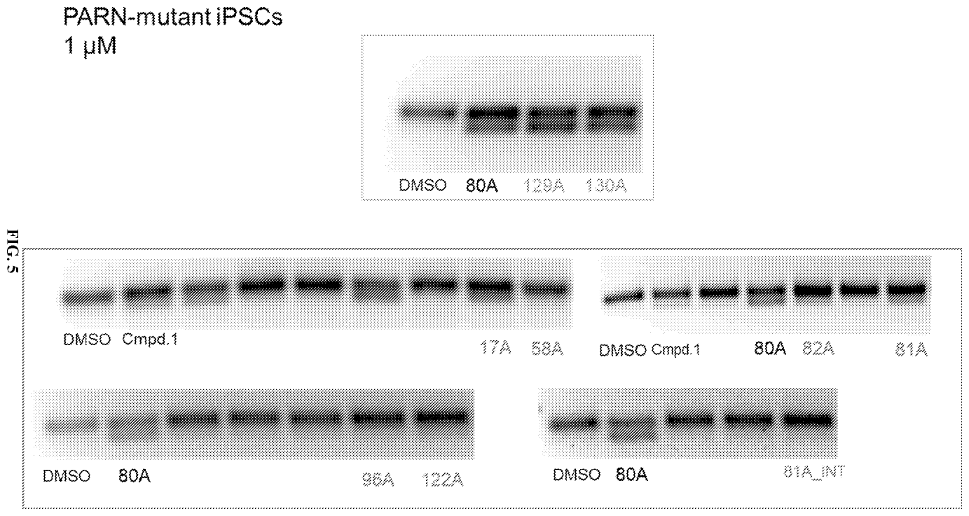

FIG. 5 shows TERC 3′ end processing—Rapid Amplification of cDNA Ends (RACE) for exemplified compounds 129A and 130A. The data is also shown for compounds 17A, 58A, 82A, 81A, 96A, 122A, and 81A-INT. The structures of compounds 17A, 58A, 82A, 81A, 96A, and 122A are shown in Example 1B. The structure of compound 81 A-INT is shown below.

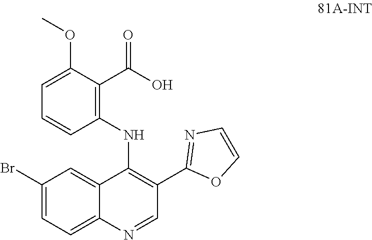 [SMILES: COc1cccc(Nc2c(-c3ncco3)cnc3ccc(Br)cc23)c1C(=O)O]

### Example 1E

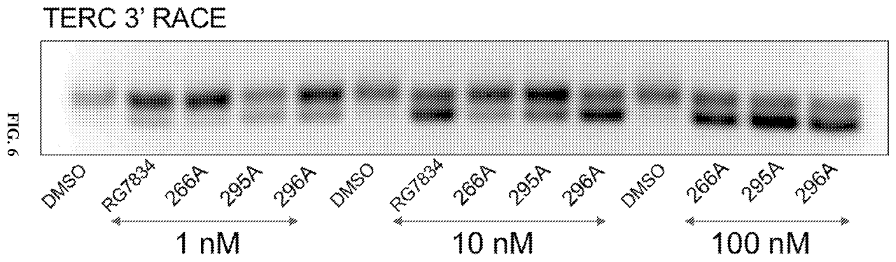

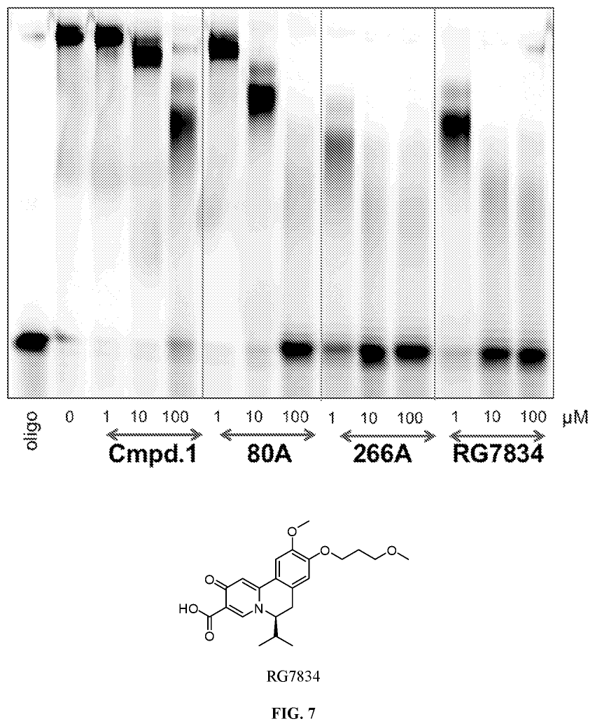

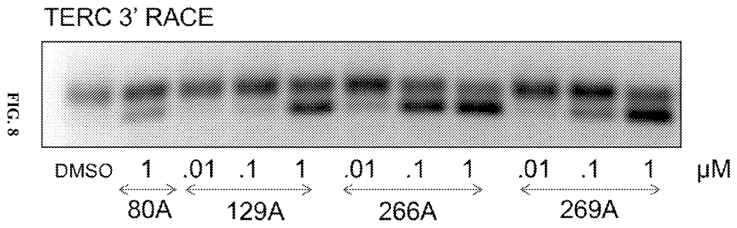

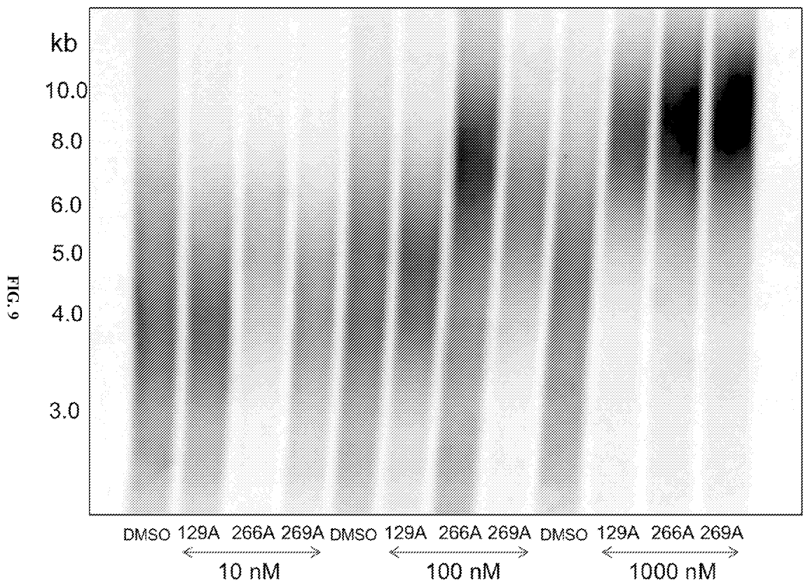

Compound 266A had activity ˜2-logs higher in the in vitro RNA oligoadenylation assay compared to the parental compound cmpd. 1, approximating the activity of RG7834 (FIG. 7). When it was tested in DC patient-based induced pluripotent stem cells (iPSCs), 266A showed the ability to drive maturation of TERC 3′ end processing by Rapid Amplification of cDNA Ends (RACE) assay at 10 nM (FIG. 8), again 1-2 logs more potently than cmpd. 1. Compounds 295A and 296A had activity 2-3 logs higher in DC patient iPSCs, showing TERC maturation at 1 nM, similar to RG7834 (FIG. 6). Consistent with this, telomere elongation in DC patient iPSCs was observed at 10 nM with 266A and at 1 nM with 295A and 296A after 3-4 weeks of cell culture (FIGS. 9 and 10). These data collectively show evidence of target engagement, and the predicted and desirable molecular activity downstream of the intended target (i.e., enhancement of TERC maturation and telomere length), in a relevant pre-clinical cellular model system, namely patient derived stem cells. Referring to Table 1D, “+” refers to activity at 1 μM, “++” refers to activity above 1 nM and below 1 μM, and “+++” refers to activity at <1 nM, “ND” refers to not determined.

TABLE 1D

| Cmpd. No. | A¹ | B² |
|---|---|---|
| 339A | +++ | +++ |
| 340A | +++ | +++ |
| 341A | ++ | ND |
| 342A | +++ | +++ |
| 343A | +++ | +++ |
| 344A | +++ | +++ |
| 345A | ++ | +++ |
| 346A | ++ | ND |
| 349A | ++ | +++ |
| 353A | ++ | +++ |
| 354A | + | ND |
| 355A | + | ND |
| 357A | + | ND |
| 361A | ++ | +++ |
| 362A | + | ND |
| 367A | + | ND |
| 368A | + | ND |
| 371A | ++ | +++ |
| 372A | + | ND |
| 376A | + | ND |
| 391A | ++ | +++ |
| 392A | +++ | +++ |
| 393A | +++ | +++ |
| 394A | +++ | ND |
| 395A | + | +++ |
| 396A | +++ | +++ |
| 399A | +++ | +++ |
| 400A | +++ | ND |
| 401A | + | ND |
| 402A | + | ND |
| 403A | + | ND |
| 404A | +++ | +++ |
| 411A | +++ | +++ |
| 413A | +++ | +++ |
| 414A | +++ | +++ |
| 415A | + | ND |
| 416A | +++ | +++ |
| 417A | +++ | +++ |
| 418A | +++ | +++ |
| 419A | +++ | +++ |
| 420A | +++ | +++ |
| 421A | +++ | +++ |
| 422A | +++ | +++ |
| 423A | +++ | +++ |
| 425A | ++ | ND |
| 426A | + | ND |
| 427A | ++ | ND |
| 428A | +++ | +++ |
| 430A | +++ | ND |

¹iPSC-based RACE activity: “+” refers to activity at 1 μM, “++” refers to activity above 1 nM and below 1 μM, and “+++” refers to activity at ≤1 nM.
²iPSC-based Telomere length: “+” refers to activity at 1 μM, “++” refers to activity above 1 nM and below 1 μM, and “+++” refers to activity at ≤1 nM, “ND” refers to not determined.

### Example 1F

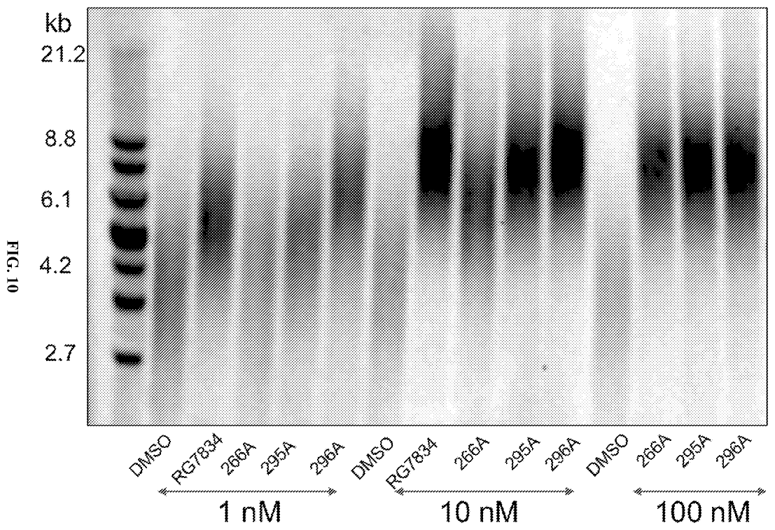

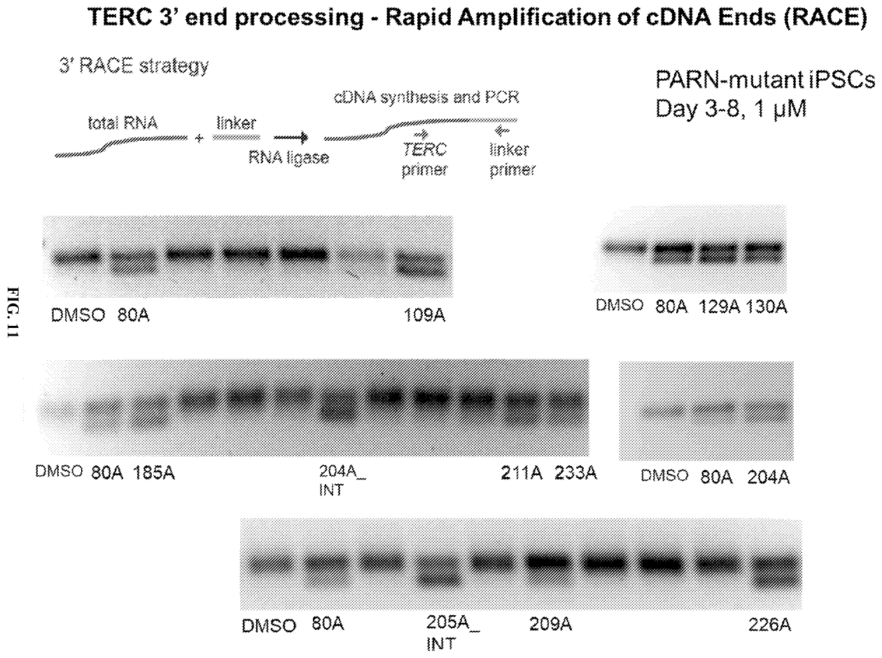

FIG. 11 shows TERC 3′ end processing—Rapid Amplification of cDNA Ends (RACE) for exemplified compounds 109A, 129A, 130A, 204A-INT, 211A, 233A, 204A, 205A-INT, 209A, and 226A. FIG. 12 shows TERC 3′ end processing—RACE for exemplified compounds 266A, 267A, 269A, 270A, 295A, 297A, 299A, 296A, 307A, 303A, 302A, 301A, 200A, 298A, 308A, 306A, 305A, 304A, 341A.

### Example 1G

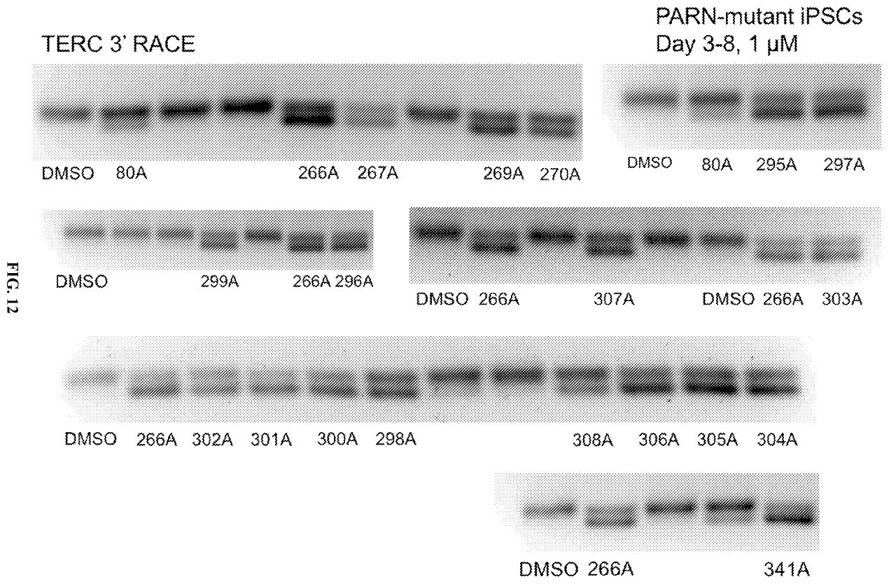

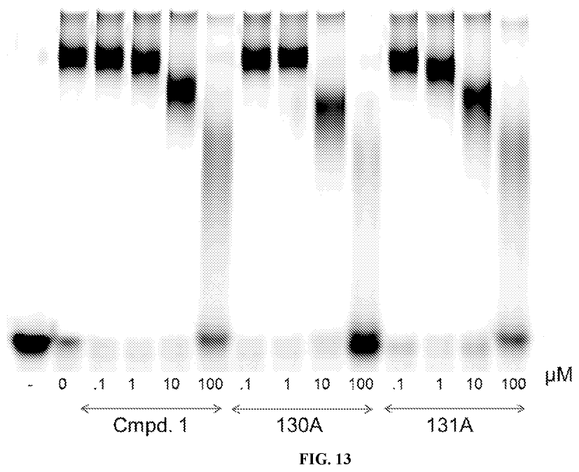

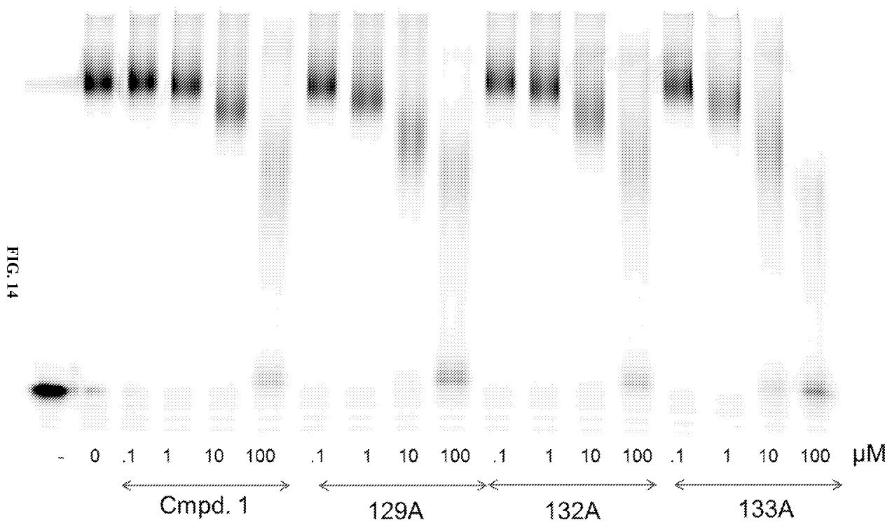

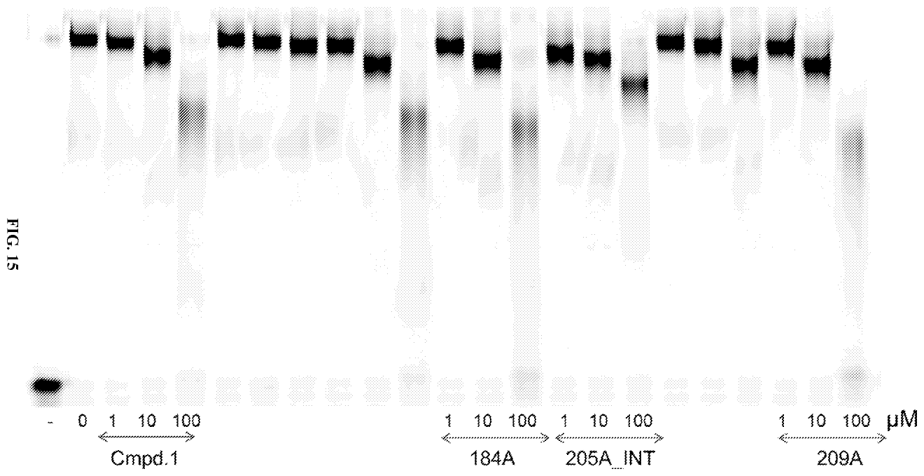

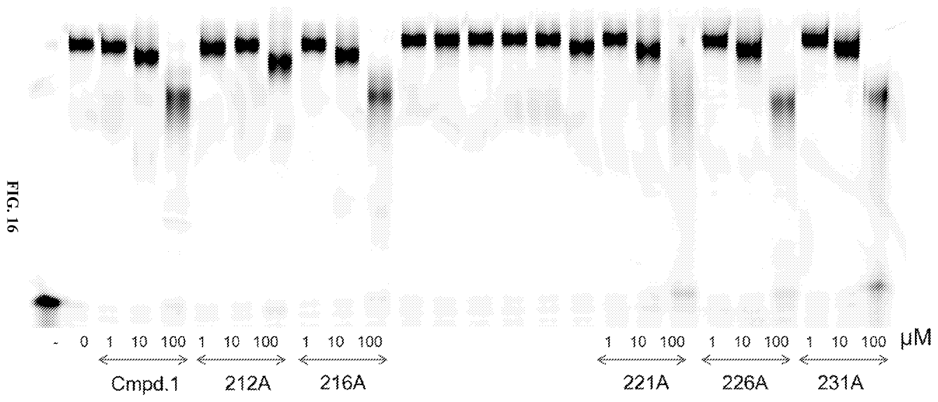

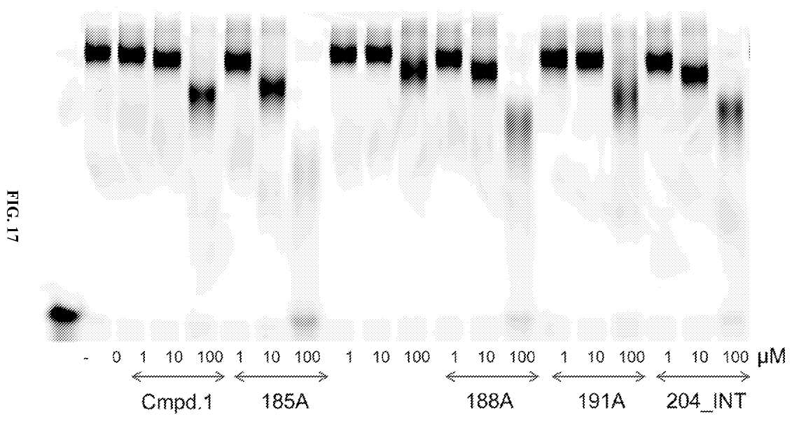

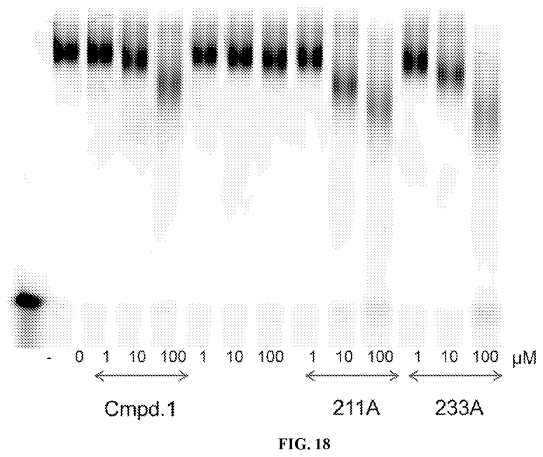

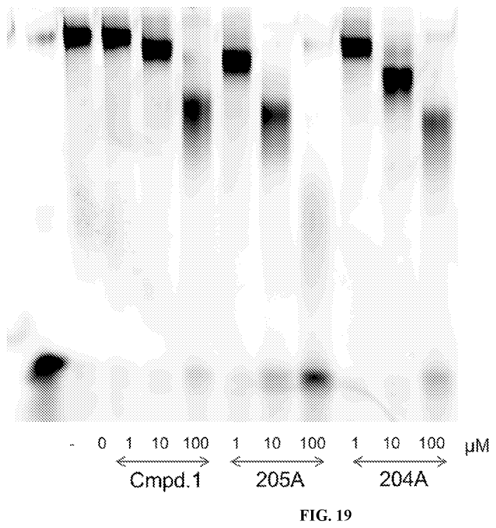

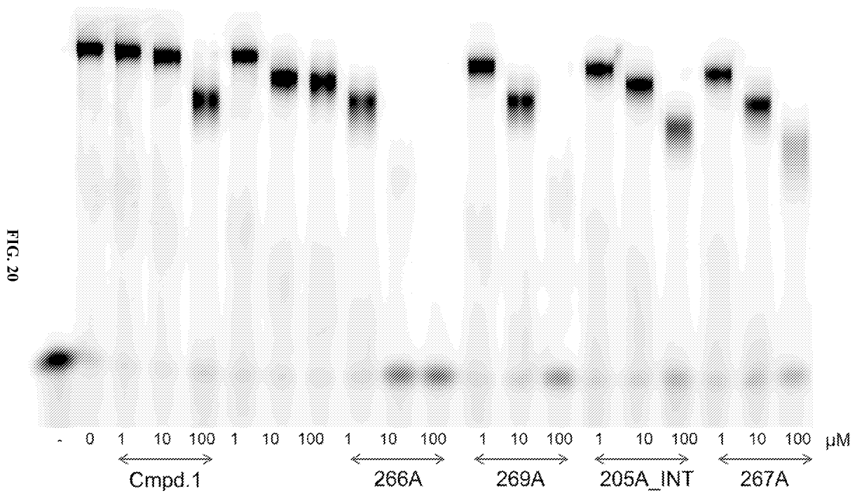

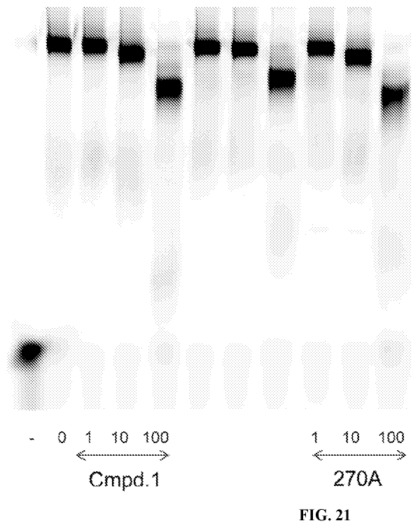

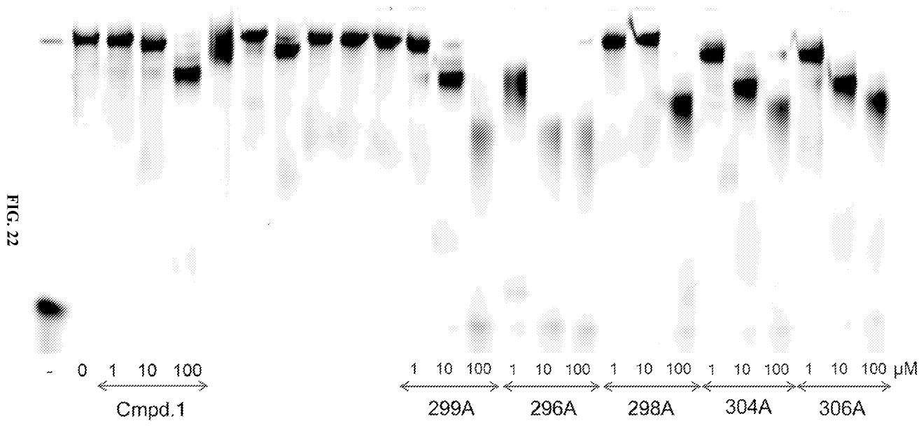

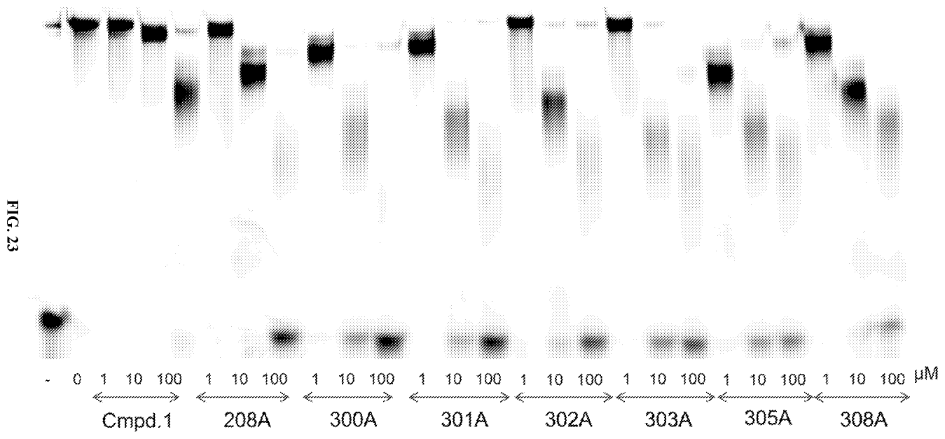

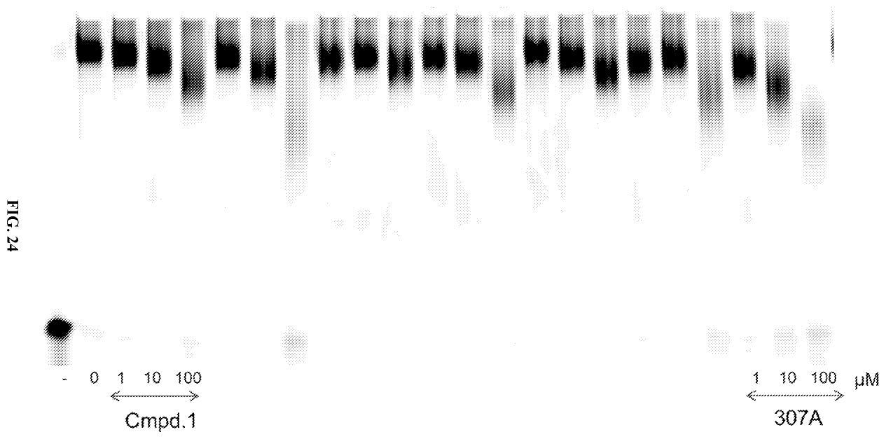

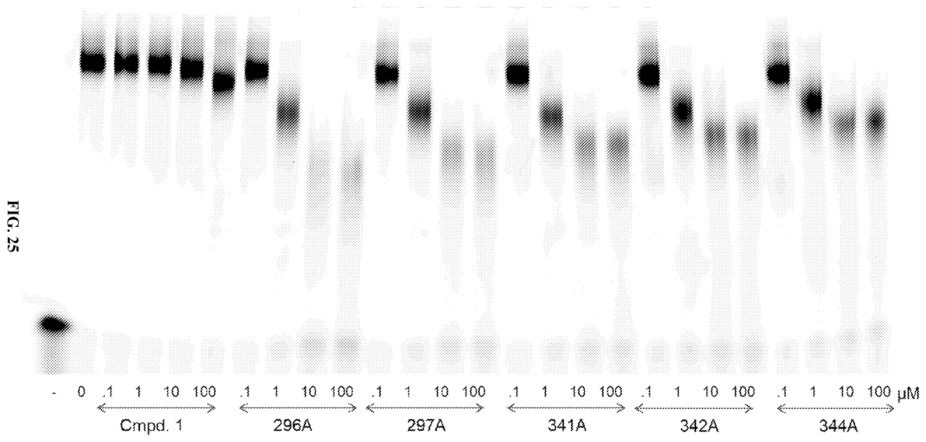

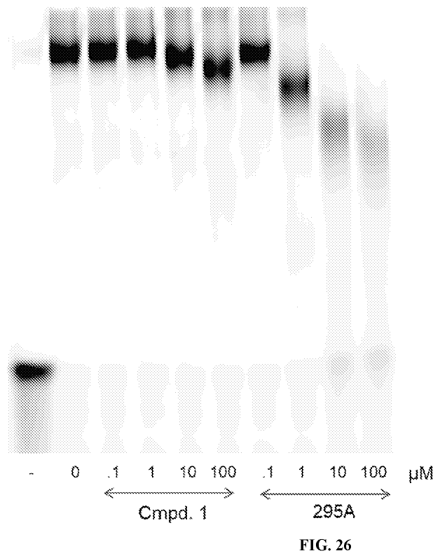

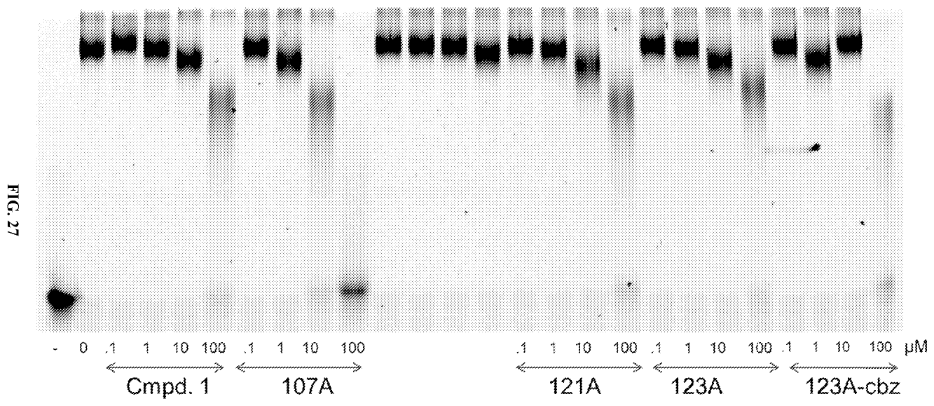

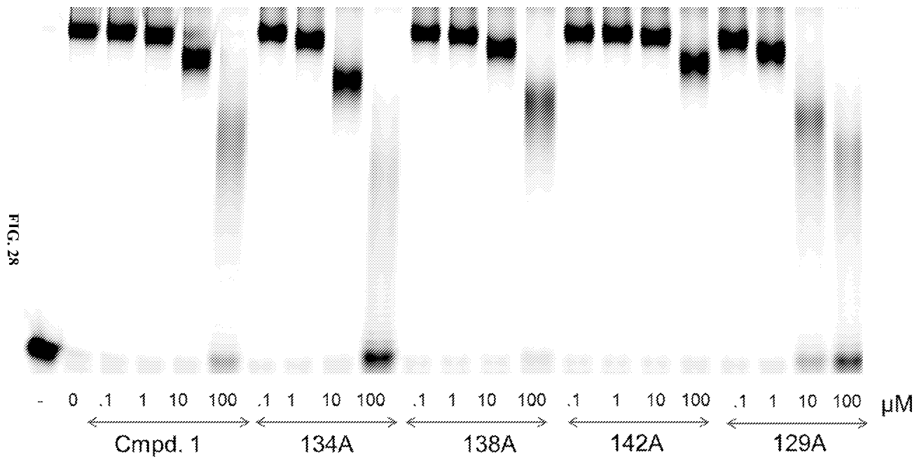

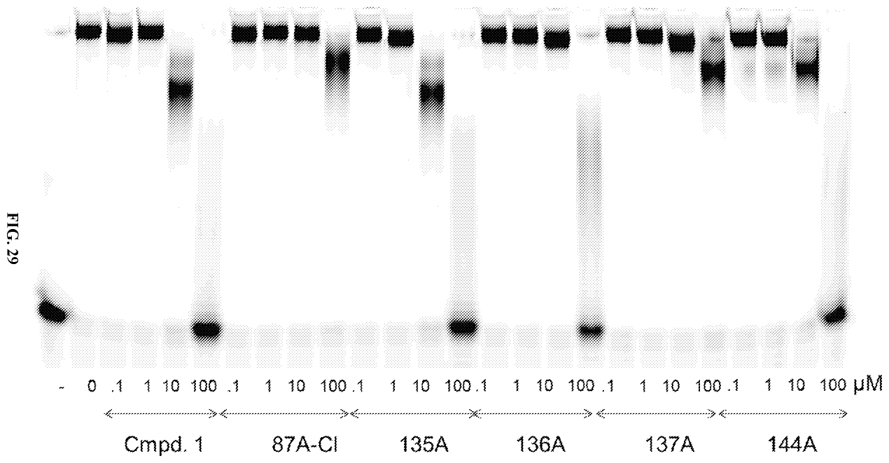

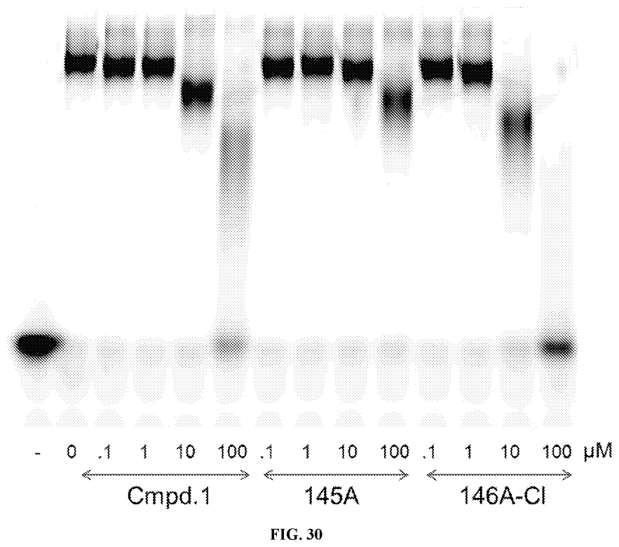

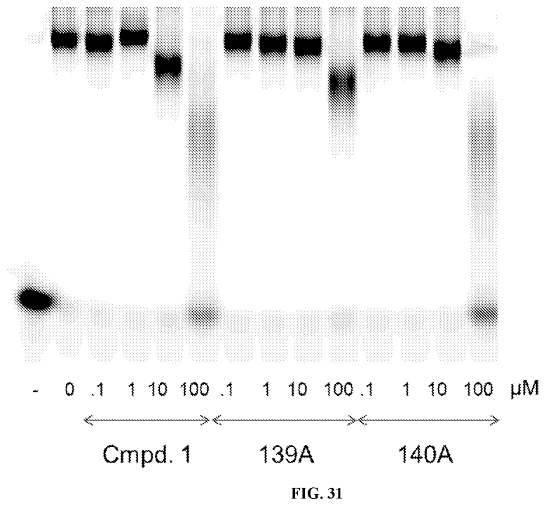

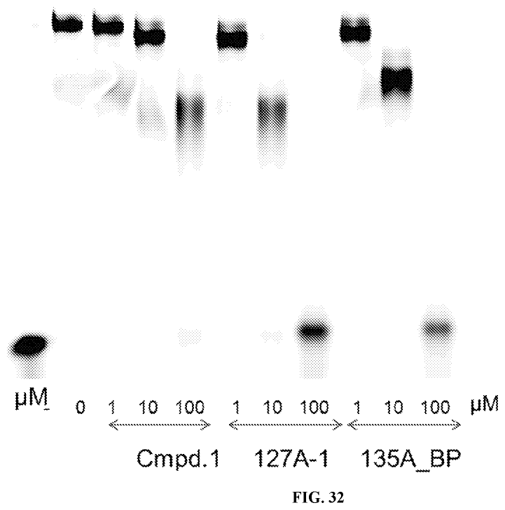

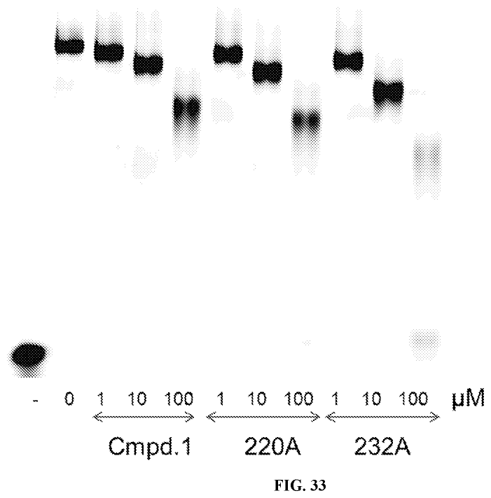

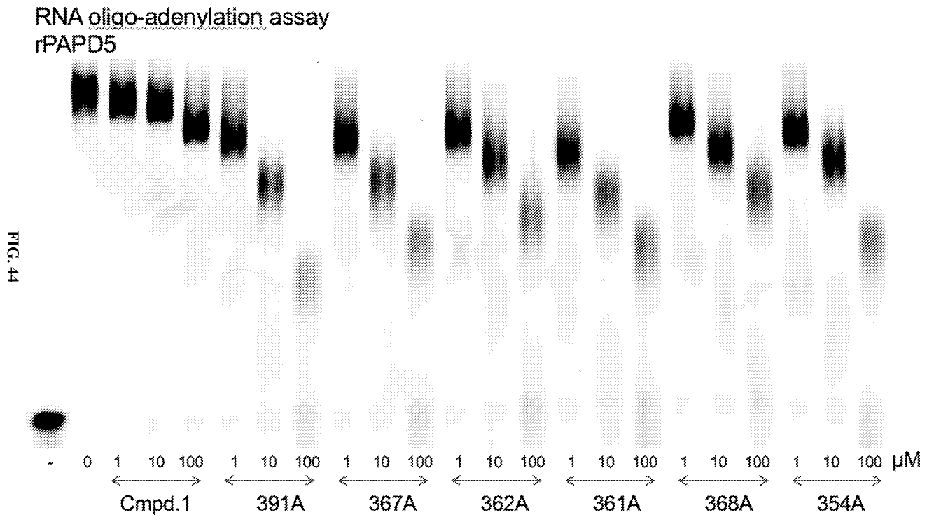

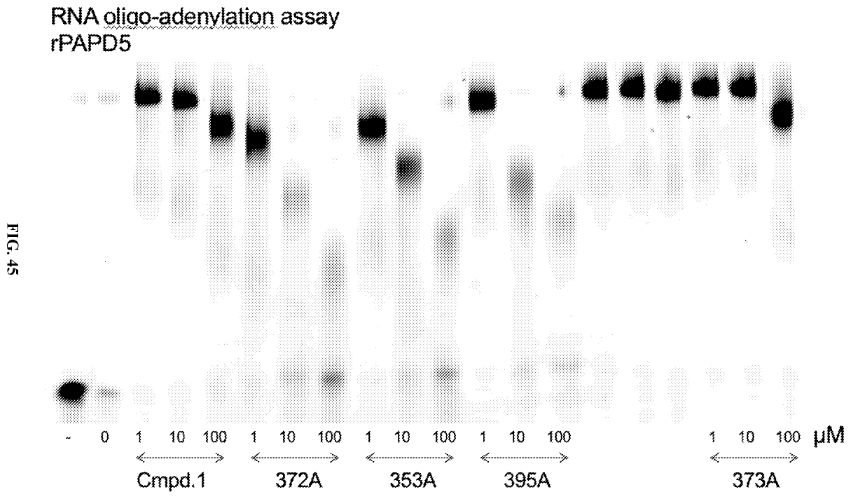

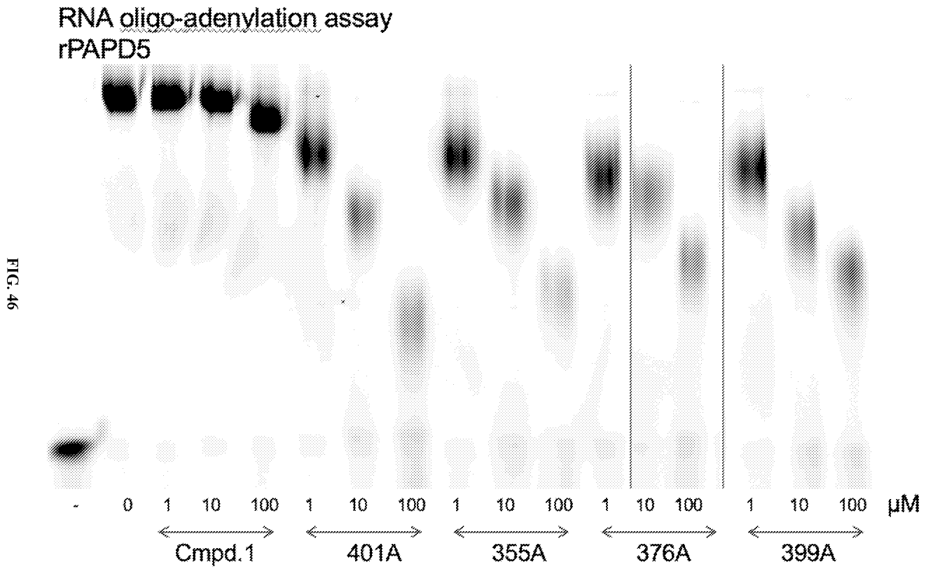

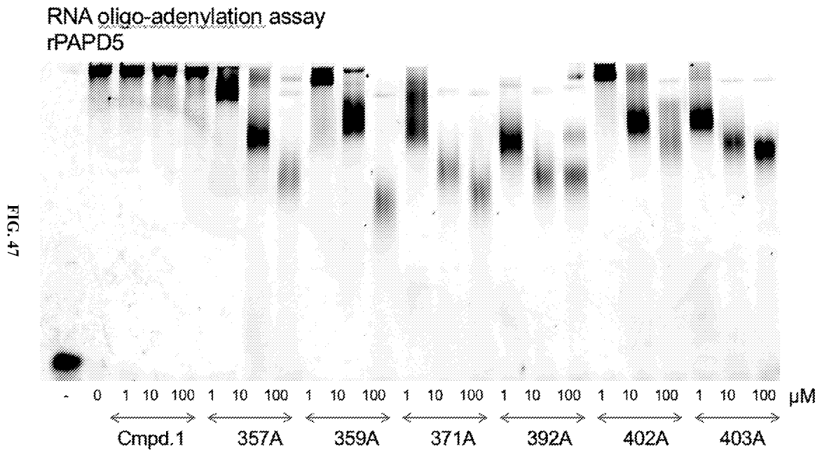

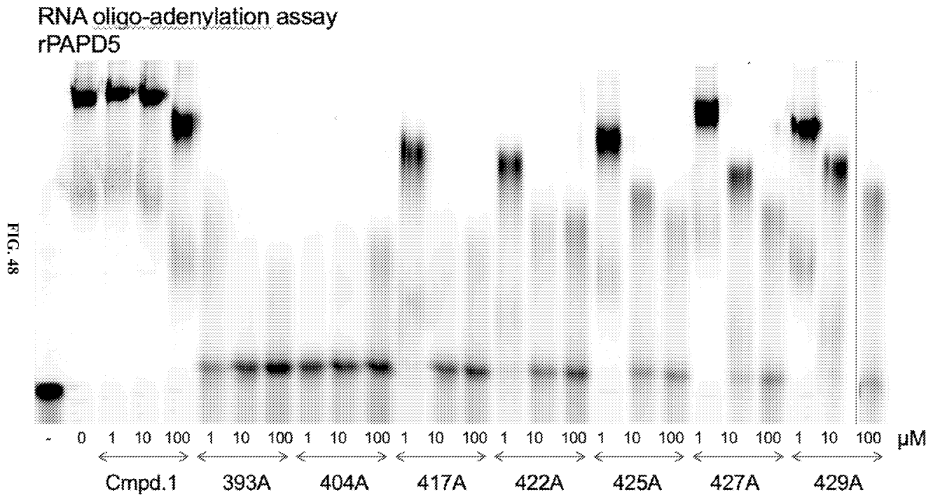

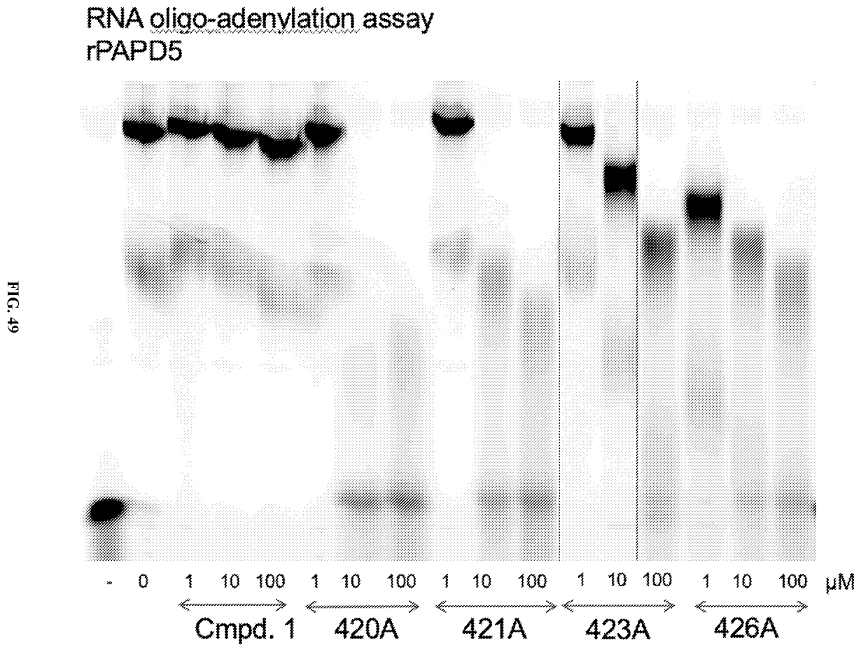

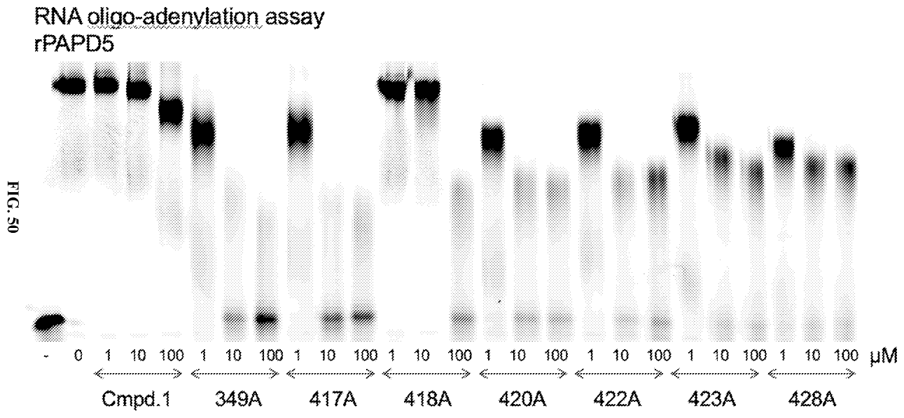

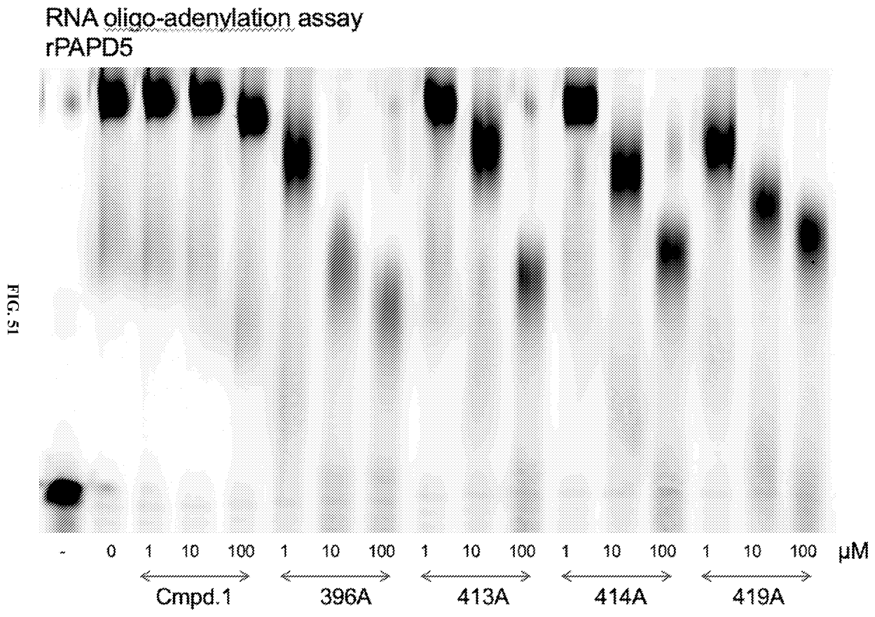

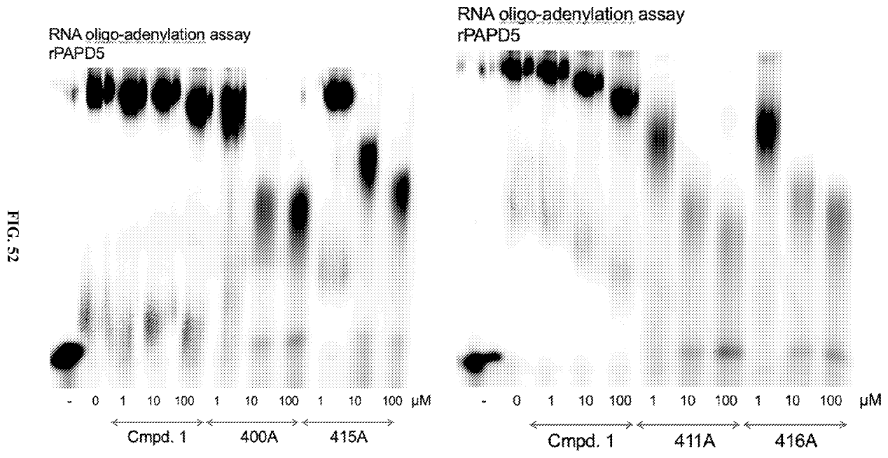

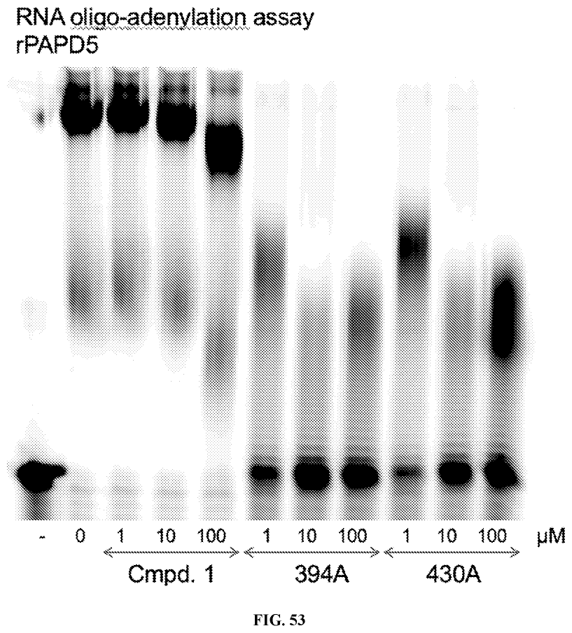

FIGS. 13-34 and 42-53 show results of RNA oligo-adenylation assay (rPAPD5) for exemplified compounds 130A, 131A, 129A, 132A, 133A, 184A, 205A-INT, 209A, 212A, 216A, 221A, 226A, 231A, 185A, 188A, 191A, 204A-INT, 211A, 233A, 205A, 204A, 266A, 269A, 205A-INT, 267A, 270A, 299A, 296A, 298A, 304A, 306A, 208A, 300A, 301A, 302A, 303A, 305A, 308A, 307A, 296A, 297A, 341A, 342A, 344A, 295A, 121A, 123A, 123A-CBZ, 134A, 138A, 142A, 129A, 87A-Cl, 135A, 136A, 137A, 144A, 145A, 146A-Cl, 139A, 140A, 127A, 135A-BP, 220A, 232A, 275A, 276A, 277A, 278A, 279A, 339A, 343A, 345A, 346A, 340A, 349A, 391A, 367A, 362A, 361A, 368A, 354A, 372A, 353A, 395A, 373A, 401A, 355A, 376A, 399A, 357A, 359A, 371A, 392A, 402A, 403A, 393A, 404A, 417A, 422A, 425A, 427A, 429A, 420A, 421A, 423A, 426A, 349A, 417A, 418A, 420A, 422A, 423A, 428A, 396A, 413A, 414A, 419A, 400A, 415A, 411A, 416A, 394A, and 430A.

### Example 1H

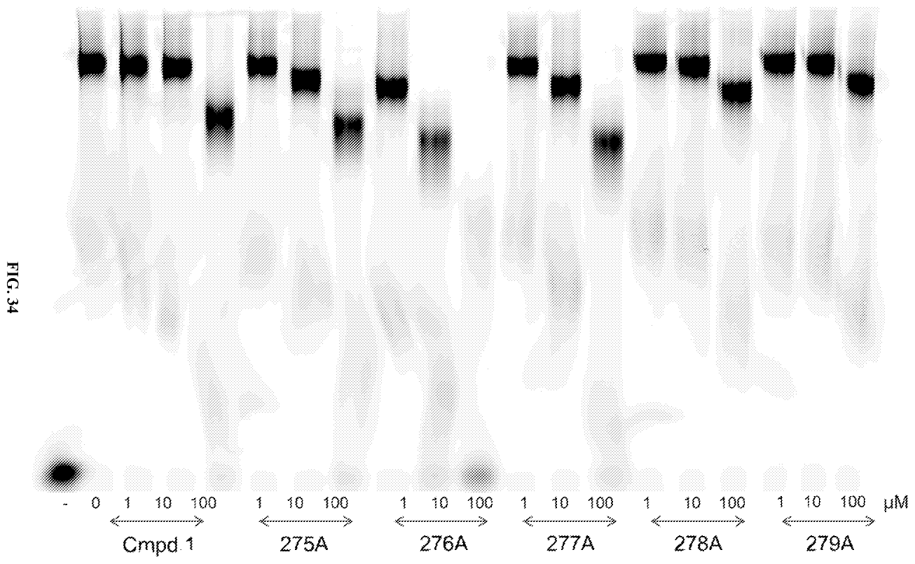

FIG. 35 shows TERC 3′ end processing—Rapid Amplification of cDNA Ends (RACE) for exemplified compounds 296A, 297A, 344A, 353A, 354A, 349A, 391A, 392A, 393A, 404A, 361A, 367A, 371A, 339A, 340A, and 343A tested at 1 nM in PARN-mutant iPSCs on day 4. FIG. 36 shows terminal restriction fragment (TRF) telomere length measurement (southern blot) for exemplified compounds 296A, 297A, 344A, 353A, 354A, 349A, 391A, 392A, 393A, 404A, 361A, 367A, 371A, 339A, 340A, and 343A tested at 1 nM in PARN-mutant iPSCs on day 4. FIG. 37 shows TERC 3′ end processing—Rapid Amplification of cDNA Ends (RACE) for exemplified compounds 296A, 349A, 399A, 411A, 416A, 417A, 418A, 420A, 421A, 422A, 423A, 428A, 396A, 413A, 414A, and 419A tested at 1 nM in PARN-mutant iPSCs on day 4. FIG. 38 shows terminal restriction fragment (TRF) telomere length measurement (southern blot) for exemplified compounds 296A, 349A, 399A, 411A, 416A, 417A, 418A, 420A, 421A, 422A, 423A, 428A, 396A, 413A, 414A, and 419A tested at 1 nM in PARN-mutant iPSCs on day 4.

### Example 2A

Binding and stabilization of rPAPD5 by test compounds was determined using differential scanning fluorimetry (DSF). DSF assays were performed to determine protein melting temperature using an indicator dye SYPRO orange (Thermo Fisher Scientific, S6651) diluted 1:5000 in 20 mL of buffer containing 20 mM rPAPD5, 100 mM non-extendable ATP analog (Jena Biosciences), 25 mM Tris-HCl, 5 mM MgCl₂, 50 mM KCl. Test compounds were added to the dye-buffer mixture at 10-100 μM and heated from 10 to 95° C. at a rate of 1° C./min and fluorescence signals were monitored by A 7500 Fast Real-Time PCR System (Applied Biosystems). DMSO was used as a negative control. Each curve was an average of three measurements and Thermal Shift software (Thermo Fisher Scientific, 4466038) was used for analysis. Results of the DSF binding assay (shown as shift in temperature at 100 μM and/or 10 μM of the test compound) are shown in the Table 2 below. The change in melting temperature (ΔTₘ) is in reference to the DMSO control. Referring to Table 2, “+” refers to ΔTₘ values below 1° C., “++” refers to ΔTₘ values from 1 to 5° C., and “+++” refers to ΔTₘ values above 5° C.

TABLE 2

| Compound No. | ΔTₘ at 100 μM |
|---|---|
| 87A-Cl | ++ |
| 91A-Cl | ++ |
| 123A | +++ |
| 123A-CBZ | +++* |
| 124A | ++ |
| 125A | ++ |
| 127A-1 | +++ |
| 128A-BR | + |
| 129A | +++ |
| 130A | +++ |
| 131A | +++ |
| 132A | +++ |
| 133A | +++ |
| 134A | +++ |
| 135A | +++ |
| 135A-BP | +++ |
| 136A | ++ |
| 137A | ++ |
| 138A | +++ |
| 139A | ++ |
| 140A | ++ |
| 141A | + |
| 142A | ++ |
| 143A | ++ |
| 144A | +++ |
| 145A | +++ |
| 146A-C1 | − |
| 147A | +++ |
| 148A-R | ++ |
| 149A | ++ |
| 150A | +++ |
| 151A | ++ |
| 152A | ++ |
| 153A | ++ |
| 154A | +++ |
| 155A | ++ |
| 156A | +++ |
| 157A | + |
| 158A | +++ |
| 159A | +++ |
| 160A | +++ |
| 161A | + |
| 162A | ++ |
| 163A | ++ |
| 164A | ++ |
| 165A | ++ |
| 166A | ++ |
| 167A | + |
| 168A | ++ |
| 169A | + |
| 170A | ++ |
| 171A | ++ |
| 172A | ++ |
| 173A | ++ |
| 174A | + |
| 175A | +++ |
| 176A | +* |
| 177A | ++ |
| 178A | ++ |
| 179A | ++ |
| 180A | + |
| 181A | ++ |
| 182A | ++ |
| 184A | +++ |
| 185A | +++ |
| 186A | ++ |
| 188A | +++ |
| 191A | +++ |
| 204A-INT | +++ |
| 204A | +++ |
| 204A-BP | +++ |
| 205A | +++ |
| 205A-INT | +++ |
| 206A | +++ |
| 206A-INT | +++ |
| 207A | +++ |
| 207A-INT | +++ |
| 208A | +++ |
| 208A-INT | ++ |
| 209A | +++ |
| 211A | +++ |
| 212A | ++ |
| 216A | +++ |
| 217A | +++ |
| 219A | ++ |
| 220A | +++ |
| 221A | +++ |
| 226A | +++ |
| 231A | +++ |
| 232A | +++ |
| 233A | +++ |
| 234A | +++ |
| 235A | ++ |
| 236A | +++ |
| 237A | + |
| 238A | ++ |
| 239A | ++ |
| 240A | ++ |
| 241A | +++ |
| 247A | ++ |
| 248A | ++ |
| 249A | +++ |
| 250A | +++ |
| 261A | + |
| 262A | + |
| 263A | +++ |
| 265A | +++ |
| 266A | +++ |
| 267A | +++ |
| 268A | + |
| 269A | +++ |
| 270A | +++ |
| 271A | ++ |
| 272A | +++ |
| 273A | ++ |
| 274A | +++ |
| 275A | +++ |
| 276A | +++ |
| 277A | +++ |
| 278A | ++ |
| 279A | ++ |
| 280A | +++ |
| 281A | +++ |
| 282A | +++ |
| 283A | +++ |
| 284A | ++ |
| 285A | + |
| 289A | +++ |
| 290A | + |
| 292A | ++ |
| 293A | ++ |
| 295A | +++ |
| 296A | +++ |
| 297A | +++ |
| 298A | +++ |
| 299A | +++ |
| 300A | +++ |
| 301A | +++ |
| 302A | +++ |
| 303A | +++ |
| 304A | +++ |
| 305A | +++ |
| 306A | +++ |
| 307A | +++ |
| 308A | +++ |
| 318A | +++ |
| 319A | ++ |
| 320A | ++ |
| 321A | + |
| 322A | +++ |
| 323A | +++ |
| 324A | +++ |
| 328A | ++ |
| 330A | +++ |
| 338A | +++ |
| 339A | +++ |
| 340A | +++* |
| 341A | +++* |
| 342A | +++* |
| 343A | +++* |
| 344A | +++* |
| 345A | +++ |
| 346A | +++ |
| 349A | +++ |
| 353A | +++ |
| 354A | +++* |
| 355A | +++ |
| 357A | +++ |
| 359A | +++ |
| 361A | +++ |
| 362A | +++ |
| 367A | +++ |
| 368A | +++ |
| 371A | +++ |
| 372A | +++ |
| 373A | ++ |
| 376A | +++ |
| 391A | +++ |
| 392A | +++* |
| 393A | +++* |
| 394A | +++* |
| 395A | +++ |
| 396A | +++ |
| 398A | + |
| 399A | +++ |
| 400A | +++* |
| 401A | +++ |
| 402A | +++ |
| 403A | +++ |
| 404A | +++* |
| 411A | +++* |
| 413A | +++* |
| 414A | +++* |
| 415A | +++ |
| 416A | +++* |
| 417A | +++* |
| 418A | +++ |
| 419A | + |
| 420A | +++* |
| 421A | +++ |
| 422A | +++* |
| 423A | +++ |
| 425A | +++ |
| 426A | +++* |
| 427A | +++ |
| 428A | +++ |
| 429A | +++ |
| 430A | +++* |

*the compound was tested at 10 μM.

TABLE 2a

| Compound No. | ΔTₘ at 10 μM | ΔTₘ at 1 μM |
|---|---|---|
| 339A | +++ | +++ |
| 340A | +++ | +++ |
| 341A | +++ | +++ |
| 342A | +++ | +++ |
| 343A | +++ | +++ |
| 344A | +++ | +++ |
| 345A | +++ | +++ |
| 346A | +++ | ++ |
| 349A | +++ | +++ |
| 353A | +++ | ++ |
| 354A | +++ | ++ |
| 355A | +++ | +++ |
| 357A | +++ | +++ |
| 359A | +++ | +++ |
| 361A | +++ | +++ |
| 362A | +++ | +++ |
| 367A | +++ | +++ |
| 368A | +++ | +++ |
| 371A | +++ | +++ |
| 372A | +++ | +++ |
| 376A | +++ | +++ |
| 391A | +++ | ++ |
| 392A | +++ | +++ |
| 393A | +++ | +++ |
| 394A | +++ | +++ |
| 395A | +++ | +++ |
| 396A | +++ | + |
| 399A | +++ | +++ |
| 400A | +++ | +++ |
| 401A | +++ | +++ |
| 402A | +++ | +++ |
| 403A | +++ | +++ |
| 404A | +++ | +++ |
| 411A | +++ | +++ |
| 413A | +++ | ++ |
| 414A | +++ | +++ |
| 415A | +++ | +++ |
| 416A | +++ | +++ |
| 417A | +++ | +++ |
| 418A | +++ | +++ |
| 419A | +++ | +++ |
| 420A | +++ | +++ |
| 421A | +++ | +++ |
| 422A | +++ | +++ |
| 423A | +++ | +++ |
| 425A | +++ | +++ |
| 426A | +++ | ++ |
| 427A | +++ | +++ |
| 428A | +++ | +++ |
| 429A | +++ | +++ |
| 430A | +++ | +++ |

Referring to Table 2a, “+” refers to ΔTₘ values below 1° C., “++” refers to ΔTₘ values from 1 to 5° C., and “+++” refers to ΔTₘ values above 5° C.

### Example 2B

HepG2.2.15 cells, a hepatitis B virus-expressing cell line (Sells, M. A. et al., PNAS, 1987), were plated at 50,000 cells/well in DMEM/F12 media (Gibco) with 10% fetal bovine serum (Omega Scientific) in a 24-well or 96-well plate (Corning), in 2-3 replicates for each test compound/concentration plus controls, and incubated in a humidified 5% CO₂ chamber at 37° C. The next day, media was aspirated, cells were washed once with phosphate buffered saline, pH7.4 (Gibco), and 1 mL DMEM/12 media was replaced. Test compounds were added in 3-fold dilutions from up to 100 μM down to 333 pM, with vehicle (dimethylsulfoxide (Sigma)) as a control. After four days of culture in a humidified 5% CO₂ chamber at 37° C., plates were spun down in a centrifuge at 300 g for 10 minutes at room temperature. Supernatant from each well was collected and either frozen at −20° C. or used directly for quantitation of hepatitis B surface antigen (HBSAg) in an enzyme-linked immunosorbent assay (ELISA). The supernatant was tested using the HBSAg ELISA kit (Abnova Cat. No KA0286) per the manufacturer's instructions. Results of replicates were averaged and nonlinear curve fitting was used to determine the half maximal inhibitory concentration (IC₅₀) for each test compound. The assay results are shown in Table 3.

TABLE 3

| Cmpd. No. | IC₅₀ (μM) |
|---|---|
| 127A | 17.9 |
| 129A | 4.6 |
| 130A | 8.89 |
| 185A | 2.19 |
| 204A | 3.39 |
| 204A_INT | 2.69 |
| 205A_INT | 1.3 |
| 226A | 9.46 |
| 233A | 29.1 |
| 266A | 0.102 |
| 267A | 4.84 |
| 269A | 1.68 |
| 270A | 4.98 |
| 295A | 0.318 |
| 296A | 0.005 |
| 297A | 0.042 |
| 298A | 1.75 |
| 301A | 0.942 |
| 303A | 0.229 |
| 305A | 0.066 |
| 306A | 1.05 |
| 307A | 1.32 |
| 339A | 0.075 |
| 340A | 0.050 |
| 341A | 0.084 |
| 342A | 0.821 |
| 343A | 0.086 |
| 344A | 0.600 |
| 345A | 0.523 |
| 346A | 0.088 |
| 349A | 0.160 |
| 353A | 0.438 |
| 354A | 3.337 |
| 361A | 0.311 |
| 367A | 0.902 |
| 371A | 0.145 |
| 376A | 0.101 |
| 391A | 0.735 |
| 392A | 0.095 |
| 393A | 0.282 |
| 404A | 0.035 |
| 417A | 0.071 |
| 418A | 0.112 |
| 420A | 0.100 |
| 428A | 0.022 |

The compounds are also useful in treating infections caused by viruses in which PAPD5/PAPD7 and/or RNA adenylation and/or guanylation is involved in viral RNA production, protein expression and/or replication. In addition to HepB, examples of these viruses include Hepatitis A (HepA) and cytomegalovirus (CMV). See Kulsuptrakul et al., A genome-wide CRISPR screen identifies UFMylation and TRAMP-like complexes as host factors required for hepatitis A virus infection, Cell Reports, 2021, 34, 108859; and Kim et al., Viral hijacking of the TENT4-ZCCHC14 complex protects viral RNAs via mixed tailing, Nature structural & molecular biology, 2020, 27, 581-588.

### Example 3—Synthesis of Compound 129A

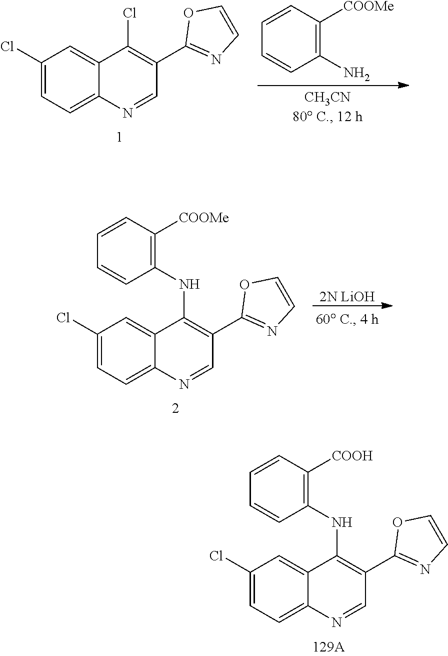

Step 1—Synthesis of methyl 2-[(6-chloro-3-oxazol-2-yl-4-quinolyl)amino]benzoate (2): A solution of 2-(4,6-dichloro-3-quinolyl)oxazole (170 mg, 641.28 umol, 1 eq) and methyl 2-aminobenzoate (96.94 mg, 641.28 umol, 82.85 uL, 1 eq) in ACN (4 mL) was stirred at 80° C. for 12 h. LCMS showed the starting material was consumed completely and desired MS was detected. The reaction mixture was concentrate in vacuum. Compound methyl 2-[(6-chloro-3-oxazol-2-yl-4-quinolyl)amino]benzoate (200 mg, crude) was obtained as a yellow solid. MS (M+H)⁺=380.2.

Step 2—Synthesis of 2-[(6-chloro-3-oxazol-2-yl-4-quinolyl)amino]benzoic acid (129 A): A solution of methyl 2-[(6-chloro-3-oxazol-2-yl-4-quinolyl)amino]benzoate (200 mg, 52 6.60 umol, 1 eq) in THF (4 mL) and LiOH·H₂O (2 M, 789.90 uL, 3 eq) was stirred at 60° C. for 4 h. LCMS showed the starting material was consumed completely and desired MS was detected. The reaction mixture was adjusted pH˜4 by adding 2N HCl. Then the mixture was purified directly. The mixture was purified by prep-HPLC (column: Phenomenex Luna 80*30 mm*3 um; mobile phase: [water(0.04% HCl)-ACN]; B %: 20%-45%,8 min). Compound 2-[(6-chloro-3-oxazol-2-yl-4-quinolyl)amino]benzoic acid (63.4 mg, 153.96 umol, 29.24% yield, 97.68% purity, HCl) was obtained as a yellow solid.

¹H NMR (400 MHz, DMSO-d6) δ=11.93-11.65 (m, 1H), 9.38 (s, 1H), 8.32 (d, J=0.8 Hz, 1H), 8.13 (br d, J=9.0 Hz, 1H), 8.03 (dd, J=1.3, 7.9 Hz, 1H), 7.98-7.91 (m, 1H), 7.70 (d, J=2.3 Hz, 1H), 7.51 (d, J=0.9 Hz, 1H), 7.48-7.40 (m, 1H), 7.35-7.26 (m, 1H), 7.01 (br d, J=8.3 Hz, 1H). MS (M+H)⁺=366.0

### Example 4—Synthesis of Compound 152A

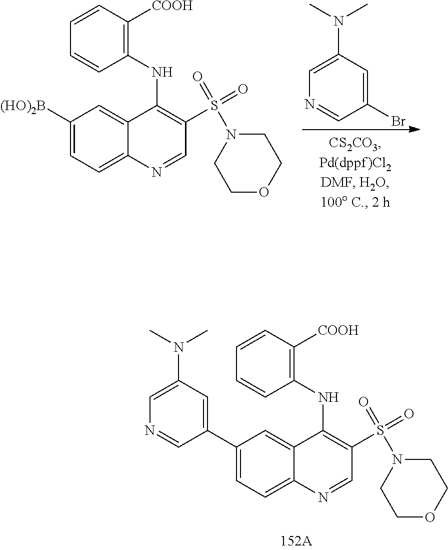

To a solution of 2-[(6-borono-3-morpholinosulfonyl-4-quinolyl)amino]benzoic acid (40 mg, 87.48 umol, 1 eq) in DMF (0.5 mL) and H₂O (0.1 mL) was added Cs₂CO₃ (85.50 mg, 262.43 umol, 3 eq), Pd(dppf)Cl2 (6.40 mg, 8.75 umol, 0.1 eq) and 5-bromo-N,N-dimethyl-pyridin-3-amine (17.59 mg, 87.48 umol, 1 eq), was bubbled with N₂ for 1 minutes, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 5%-25%,8 min) Compound 2-[[6-[5-(dimethylamino)-3-pyridyl]-3-morpholinosulfonyl-4-quinolyl]amino]benzoic acid (4.60 mg, 8.02 umol, 9.17% yield, 99.39% purity, HCl) was obtained as a yellow solid. ¹H NMR (400 MHz, DMSO-d₆) δ ppm 10.64 (br s, 1H) 9.12 (s, 1H), 8.31-8.36 (m, 1H), 8.23-8.28 (m, 1H), 8.16-8.21 (m, 1H), 8.15 (s, 1H), 8.01 (dd, J=7.82, 1.44 Hz, 1H), 7.97 (d, J=1.63 Hz, 1H), 7.34-7.41 (m, 1H), 7.30 (s, 1H), 7.09 (t, J=7.50 Hz, 1H), 6.84 (d, J=8.00 Hz, 1H), 3.49-3.54 (m, 2H), 3.41-3.46 (m, 2H), 3.06-3.16 (m, 4H), 3.01 (s, 6H). MS (M+H)⁺=534.1

### Example 5—Synthesis of Compound 153A

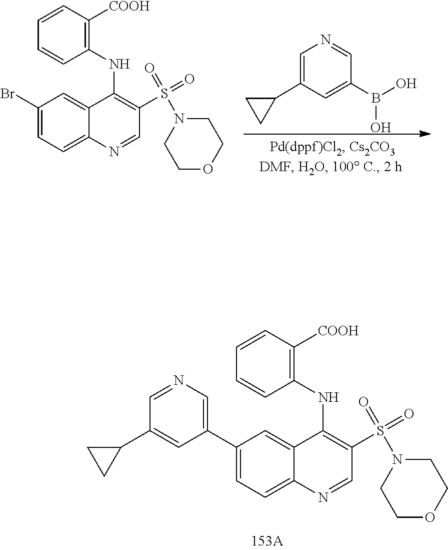 [SMILES: O=C(O)c1ccccc1Nc1c(S(=O)(=O)N2CCOCC2)cnc2ccc(Br)cc12 ; OB(O)c1cncc(C2CC2)c1 ; O=C(O)c1ccccc1Nc1c(S(=O)(=O)N2CCOCC2)cnc2ccc(-c3cncc(C4CC4)c3)cc12]

To a solution of 2-[(6-bromo-3-morpholinosulfonyl-4-quinolyl)amino]benzoic acid (40 mg, 81.24 umol, 1 eq) in H₂O (0.2 mL) and DMF (1 mL) was added Pd(dppf)Cl₂ (5.94 mg, 8.12 umol, 0.1 eq), Cs₂CO₃ (79.41 mg, 243.73 umol, 3 eq) and (5-cyclopropyl-3-pyridyl) boronic acid (13.24 mg, 81.24 umol, 1 eq), was bubbled with N₂ for 1 minute, the mixture was stirred at 100° C. for 2 h. LCMS showed the starting material was consumed completely and the MS of desired product was detected. The reaction mixture was concentrate in vacuum. The residue was purified by prep-HPLC (column: Welch Xtimate C18 100*25 mm*3 um; mobile phase: [water(0.05% HCl)-ACN]; B %: 10%-40%,8 min) Compound 2-[[6-(5-cyclopropyl-3-pyridyl)-3-morpholinosulfonyl-4-quinolyl]amino]benzoic acid (23.70 mg, 39.95 umol, 49.17% yield, 95.59% purity, HCl) was obtained as yellow solid. ¹H NMR (400 MHz, DMSO-d₆) δ ppm=10.76 (br s, 1H), 9.13 (s, 1H), 8.70 (s, 1H), 8.64 (s, 1H), 8.34-8.41 (m, 1H), 8.26-8.32 (m, 1H), 8.03-8.09 (m, 1H), 7.94 (s, 1H), 7.65 (s, 1H), 7.43 (t, J=7.76 Hz, 1H), 7.22 (t, J=7.46 Hz, 1H), 6.97 (br d, J=8.19 Hz, 1H), 3.51-3.60 (m, 2H), 3.41-3.50 (m, 2H), 3.04-3.22 (m, 4H), 2.07-2.17 (m, 1H), 1.15 (br d, J=8.31 Hz, 2H), 0.84 (dd, J=4.71, 1.65 Hz, 2H). MS (M+H)⁺=531.2

### Example 6—Synthesis of Compound 154A

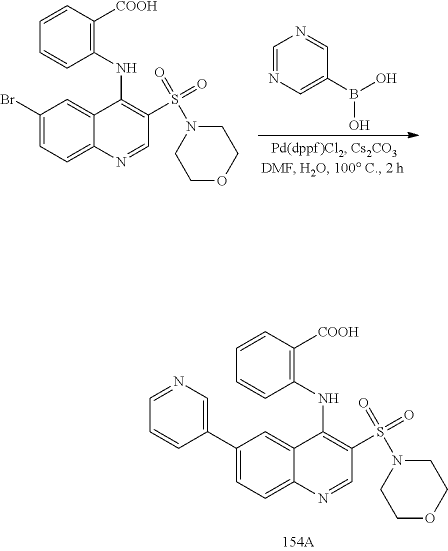 [SMILES: O=C(O)c1ccccc1Nc1c(S(=O)(=O)N2CCOCC2)cnc2ccc(Br)cc12 ; OB(O)c1cncnc1 ; O=C(O)c1ccccc1Nc1c(S(=O)(=O)N2CCOCC2)cnc2ccc(-c3cccnc3)cc12]

To a solution of 2-[(6-bromo-3-morpholinosulfonyl-4-quinolyl)amino]benzoic acid (40 mg, 81.24 umol, 1 eq) in DMF (1 mL) and H₂O (0.2 mL) was added Cs₂CO₃ (79.41 mg, 243.73 umol, 3 eq), Pd(dppf)Cl₂ (5.94 mg, 8.12 umol, 0.1 eq) and pyrimidin-5-ylboronic acid (10.07 mg, 81.24 umol, 1 eq), was bubbled with N₂ for 1 minutes, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min). Compound 2-[(3-morpholinosulfonyl-6-pyrimidin-5-yl-4-quinolyl)amino]benzoic acid (18.20 mg, 31.90 umol, 39.26% yield, 92.53% purity, HCl) was obtained as yellow solid. ¹H NMR (400 MHz, DMSO-d₆) δ ppm=10.83 (br s, 1H), 9.16 (d, J=9.54 Hz, 2H), 8.73 (s, 2H), 8.35-8.41 (m, 1H), 8.29-8.34 (m, 1H), 8.04-8.10 (m, 1H), 7.92 (d, J=1.59 Hz, 1H), 7.41-7.47 (m, 1H), 7.25 (t, J=7.52 Hz, 1H), 7.04 (br d, J=8.31 Hz, 1H), 3.53-3.61 (m, 2H), 3.42-3.51 (m, 2H), 3.08-3.24 (m, 4H). MS (M+H)⁺=492.2

### Example 7—Synthesis of Compound 155A

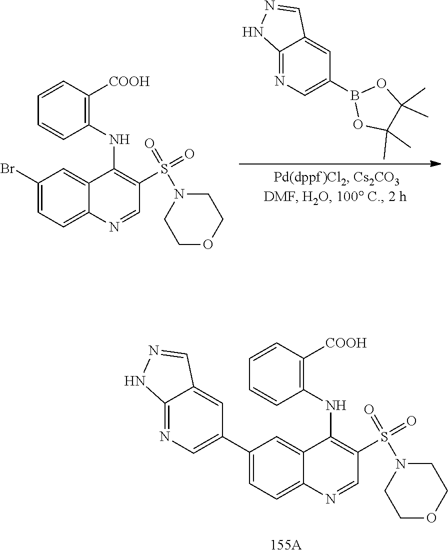 [SMILES: O=C(O)c1ccccc1Nc1c(S(=O)(=O)N2CCOCC2)cnc2ccc(Br)cc12 ; CC1(C)OB(c2cnc3[nH]ncc3c2)OC1(C)C ; O=C(O)c1ccccc1Nc1c(S(=O)(=O)N2CCOCC2)cnc2ccc(-c3cnc4[nH]ncc4c3)cc12]

To a solution of 2-[(6-bromo-3-morpholinosulfonyl-4-quinolyl)amino]benzoic acid (40 mg, 81.24 umol, 1 eq) in H₂O (0.2 mL) and DMF (1 mL) was added Cs₂CO₃ (79.41 mg, 243.73 umol, 3 eq), Pd(dppf)Cl₂ (5.94 mg, 8.12 umol, 0.1 eq) and 5-(4,4,5,5-tetramethyl-1,3,2-dioxaborolan-2-yl)-1H-pyrazolo[3,4-b]pyridine (19.91 mg, 81.24 umol, 1 eq), was bubbled with N₂ for 1 minutes, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 15%-40%,8 min). Compound 2-[[3-morpholinosulfonyl-6-(1H-pyrazolo[3,4-b]pyridin-5-yl)-4-quinolyl]amino]benzoic acid (4.60 mg, 8.11 umol, 9.99% yield, 100% purity, HCl) was obtained as yellow solid. ¹H NMR (400 MHz, DMSO-d₆) δ ppm=13.80 (br s, 1H), 10.63 (br s, 1H), 9.11 (s, 1H), 8.37 (d, J=2.08 Hz, 1H), 8.28-8.33 (m, 1H), 8.21-8.25 (m, 2H), 8.19 (s, 1H), 8.03-8.09 (m, 1H), 7.84 (d, J=1.59 Hz, 1H), 7.44 (t, J=7.09 Hz, 1H), 7.20 (t, J=7.46 Hz, 1H), 6.90 (d, J=8.31 Hz, 1H), 3.51-3.57 (m, 2H), 3.39-3.48 (m, 2H), 3.02-3.20 (m, 4H). MS (M+H)⁺=531.1

### Example 8—Synthesis of Compound 156A

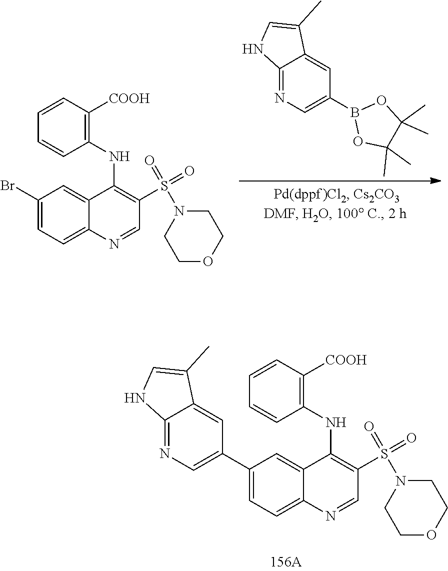 [SMILES: O=C(O)c1ccccc1Nc1c(S(=O)(=O)N2CCOCC2)cnc2ccc(Br)cc12 ; Cc1c[nH]c2ncc(B3OC(C)(C)C(C)(C)O3)cc12 ; Cc1c[nH]c2ncc(-c3ccc4ncc(S(=O)(=O)N5CCOCC5)c(Nc5ccccc5C(=O)O)c4c3)cc12]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and 3-methyl-5-(4,4,5,5-tetramethyl-1,3,2-dioxaborolan-2-yl)-1H-pyrrolo[2,3-b]pyridine (18.35 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min). Compound 2-[[6-(3-methyl-TH-pyrrolo[2,3-b]pyridin-5-yl)-3-morpholinosulfonyl-4-quinolyl]amino]benzoic acid (9.40 mg, 15.88 umol, 22.34% yield, 97.98% purity, HCl) was obtained as a yellow solid. ¹H NMR (400 MHz, DMSO-d₆) δ ppm=11.57 (s, 1H), 10.71 (br s, 1H), 9.12 (s, 1H), 8.37 (dd, J=8.82, 1.94 Hz, 1H), 8.19-8.28 (m, 2H), 8.11 (dd, J=7.88, 1.50 Hz, 1H), 7.80 (d, J=1.88 Hz, 1H), 7.69 (d, J=2.00 Hz, 1H), 7.51-7.59 (m, 1H), 7.27-7.37 (m, 2H), 7.09 (d, J=8.25 Hz, 1H), 3.56-3.62 (m, 2H), 3.46-3.54 (m, 2H), 3.10-3.26 (m, 4H), 2.26 (s, 3H). MS (M+H)⁺=544.3

### Example 9—Synthesis of Compound 157A

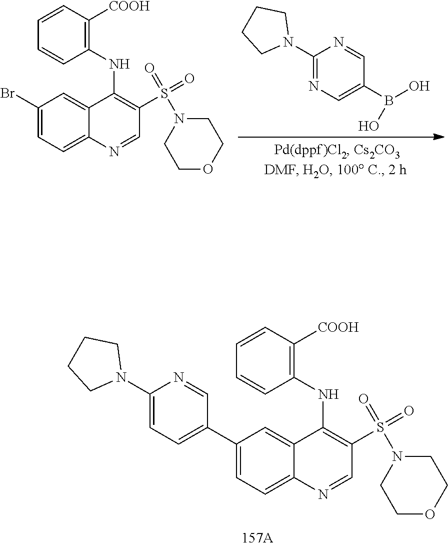 [SMILES: O=C(O)c1ccccc1Nc1c(S(=O)(=O)N2CCOCC2)cnc2ccc(Br)cc12 ; OB(O)c1cnc(N2CCCC2)nc1 ; O=C(O)c1ccccc1Nc1c(S(=O)(=O)N2CCOCC2)cnc2ccc(-c3ccc(N4CCCC4)nc3)cc12]

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and (6-pyrrolidin-1-yl-3-pyridyl)boronic acid (15.60 mg, 81.24 umol, 1 eq) was bubbled with N₂ for 1 minute, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase:[water(0.05% HCl)-ACN]; B %: 10%-40%,8 min). Compound 2-[[3-morpholinosulfonyl-6-(6-pyrrolidin-1-yl-3-pyridyl)-4-quinolyl]amino]benzoic acid (10.20 mg, 16.61 umol, 20.44% yield, 97.05% purity, HCl) was obtained as yellow solid. ¹H NMR (400 MHz, DMSO-d₆) δ ppm=10.67 (br s, 1H), 9.10 (s, 1H), 8.20-8.29 (m, 2H), 8.03 (d, J=6.72 Hz, 1H), 7.94 (s, 1H), 7.87 (br d, J=9.29 Hz, 1H), 7.78 (s, 1H), 7.36 (t, J=7.21 Hz, 1H), 7.08-7.19 (m, 2H), 6.83 (d, J=8.19 Hz, 1H), 3.49-3.64 (m, 6H), 3.37-3.47 (m, 2H), 3.03-3.18 (m, 4H), 2.01 (br s, 4H). MS (M+H)⁺=560.2

### Example 10—Synthesis of Compound 158A

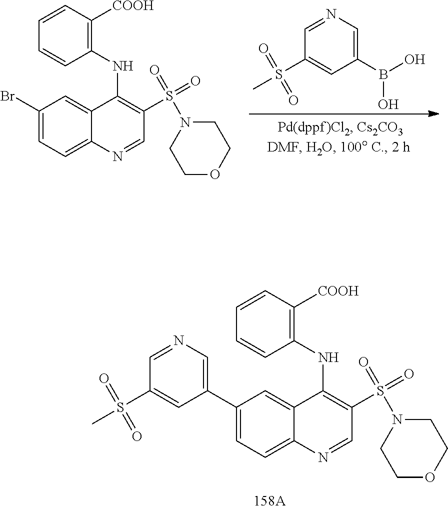 [SMILES: O=C(O)c1ccccc1Nc1c(S(=O)(=O)N2CCOCC2)cnc2ccc(Br)cc12 ; CS(=O)(=O)c1cncc(B(O)O)c1 ; CS(=O)(=O)c1cncc(-c2ccc3ncc(S(=O)(=O)N4CCOCC4)c(Nc4ccccc4C(=O)O)c3c2)c1]

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and (4-benzyloxy-2-methyl-phenyl)boronic acid (19.67 mg, 81.24 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 30%-50%,8 min). Compound 2-[[6-(4-benzyloxy-2-methyl-phenyl)-3-morpholinosulfonyl-4-quinolyl]amino]benzoic acid (9.10 mg, 14.04 umol, 17.28% yield, 99.66% purity, HCl) was obtained as a yellow solid. ¹H NMR (400 MHz, DMSO-d₆+D₂O) δ ppm=9.08 (s, 1H), 9.00 (d, J=2.00 Hz, 1H), 8.74 (d, J=2.13 Hz, 1H), 8.31 (dd, J=8.88, 2.00 Hz, 1H), 8.21 (d, J=8.76 Hz, 1H), 8.15 (t, J=2.13 Hz, 1H), 8.03 (dd, J=7.94, 1.56 Hz, 1H), 7.87 (d, J=1.88 Hz, 1H), 7.37-7.43 (m, 1H), 7.15-7.21 (m, 1H), 6.88 (d, J=7.88 Hz, 1H), 3.46-3.55 (m, 2H), 3.37-3.46 (m, 2H), 3.28 (s, 3H), 3.00-3.16 (m, 4H). MS (M+H)⁺=569.1

### Example 11—Synthesis of Compound 159A

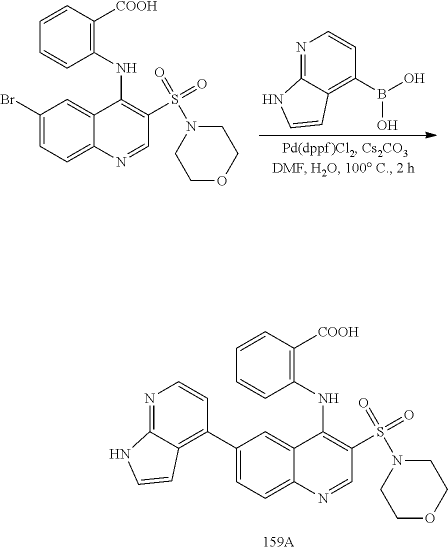 [SMILES: O=C(O)c1ccccc1Nc1c(S(=O)(=O)N2CCOCC2)cnc2ccc(Br)cc12 ; OB(O)c1ccnc2[nH]ccc12 ; O=C(O)c1ccccc1Nc1c(S(=O)(=O)N2CCOCC2)cnc2ccc(-c3ccnc4[nH]ccc34)cc12]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and 1Hpyrrolo[2,3-b]pyridin-4-ylboronic acid (11.51 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product purified by prep-HPLC (column: Welch Xtimate C18 100*25 mm*3 um; mobile phase: [water(0.05% HCl)-ACN]; B %: 10%-30%, 8 min). Compound 2-[[3-morpholinosulfonyl-6-(1H-pyrrolo[2,3-b]pyridin-4-yl)-4-quinolyl]amino]benzoic acid (12.40 mg, 21.76 umol, 30.62% yield, 99.35% purity, HCl) was obtained as a yellow solid. ¹H NMR (400 MHz, DMSO-d₆) δ ppm=12.20 (br s, 1H), 10.51 (br s, 1H), 9.17 (s, 1H), 8.33 (s, 2H), 8.30 (d, J=5.38 Hz, 1H), 8.10 (s, 1H), 8.03 (dd, J=7.94, 1.56 Hz, 1H), 7.41-7.54 (m, 2H), 7.21 (t, J=7.57 Hz, 1H), 7.08 (d, J=5.25 Hz, 1H), 7.00 (br d, J=8.25 Hz, 1H), 6.04 (d, J=1.75 Hz, 1H), 3.49-3.60 (m, 2H), 3.38-3.49 (m, 2H), 3.06-3.19 (m, 4H). MS (M+H)⁺=530.3

### Example 12—Synthesis of Compound 160A

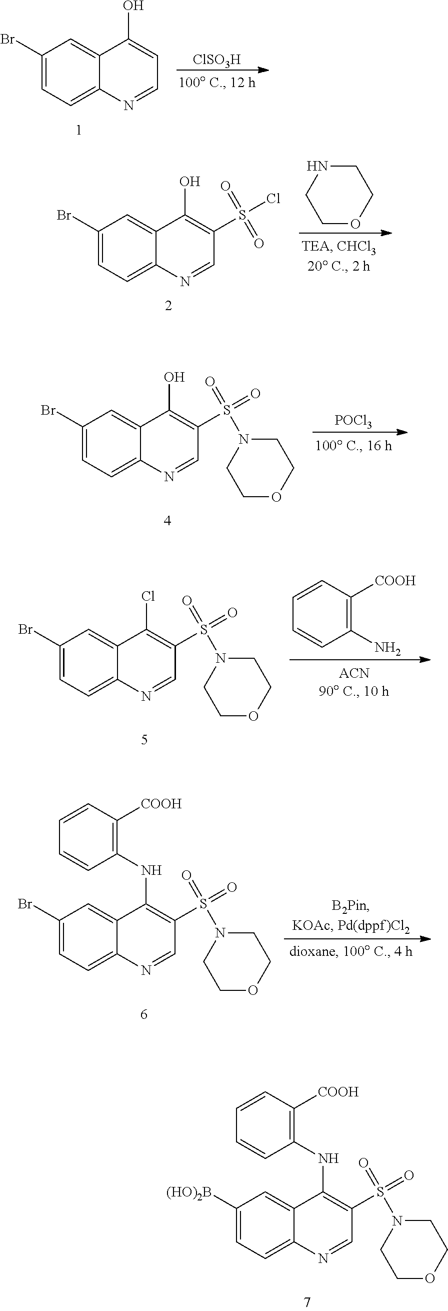

Step 1—Synthesis of 6-bromo-4-hydroxy-quinoline-3-sulfonyl chloride (2): A solution of 6-bromoquinolin-4-ol (6.24 g, 27.84 mmol, 1 eq) in HSO₃Cl (20 mL) was stirred at 100° C. for 12 h. LCMS showed the starting material was consumed completely and desired MS was detected. The mixture was added dropwise into ice water (˜10 mL). Filtered, and filter cake was concentrate in vacuum. Compound 6-bromo-4-hydroxy-quinoline-3-sulfonyl chloride (7 g, 21.70 mmol, 77.95% yield) was obtained as a black solid. MS (M+H)⁺=323.9.

Step 2. Synthesis of 6-bromo-3-morpholinosulfonyl-quinolin-4-ol (4): To a solution of 6-bromo-4-hydroxy-quinoline-3-sulfonyl chloride (7 g, 21.70 mmol, 1 eq) in DCM (70 mL) was added TEA (6.59 g, 65.10 mmol, 9.06 mL, 3 eq) and morpholine (2.08 g, 23.87 mmol, 2.10 mL, 1.1 eq) was stirred at 25° C. for 2 h. LCMS showed the starting material was consumed completely and the MS of desired product was detected. The reaction mixture was concentrate in vacuum. Compound 6-bromo-3-morpholinosulfonyl-quinolin-4-ol (3 g, 8.04 mmol, 37.04% yield) was obtained as white solid. MS (M+H)⁺=373.0.

Step 3. Synthesis of 4-[(6-bromo-4-chloro-3-quinolyl)sulfonyl]morpholine (5): A solution of 6-bromo-3-morpholinosulfonyl-quinolin-4-ol (3 g, 8.04 mmol, 1 eq) in POCl₃ (24.75 g, 161.42 mmol, 15 mL, 20.08 eq) was stirred at 100° C. for 16 h. TLC (Petroleum ether/Ethyl acetate=3:1, R_{f}=0.41) showed starting material was consumed completely and new spot was formed. The reaction mixture was poured into water (20 mL) The aqueous phase was extracted with dichloromethane (50 mL*2). The combined organic phase was dried with anhydrous Na₂SO₄, filtered and concentrated in vacuum. The residue was purified by flash column (ISCO 20 g silica, 30-36% Ethyl acetate in Petroleum ether, gradient over 15 min). Compound 4-[(6-bromo-4-chloro-3-quinolyl)sulfonyl]morpholine (1.6 g, 4.09 mmol, 50.82% yield) was obtained as a yellow solid.

Step 4. Synthesis of 2-[(6-bromo-3-morpholinosulfonyl-4-quinolyl)amino]benzoic acid (6): A solution of 2-aminobenzoic acid (560.21 mg, 4.09 mmol, 1 eq),4-[(6-bromo-4-chloro-3-quinolyl)sulfonyl]morpholine (1.6 g, 4.09 mmol, 1 eq) in ACN (20 mL) was stirred at 80° C. for 2 h. LCMS showed the starting material was consumed completely and desired MS was detected. The reaction mixture was concentrate in vacuum. Compound 2-[(6-bromo-3-morpholinosulfonyl-4-quinolyl)amino]benzoic acid (2 g, 4.06 mmol, 99.44% yield) was obtained as yellow solid. MS (M+H)⁺=494.0.

Step 5. Synthesis of 2-[(6-borono-3-morpholinosulfonyl-4-quinolyl)amino]benzoic acid (7): To a stirred solution of 2-[(6-bromo-3-morpholinosulfonyl-4-quinolyl)amino]benzoic acid (500 mg, 1.02 mmol, 1 eq) in dioxane (10 mL) was added BPD (309.46 mg, 1.22 mmol, 1.2 eq), Pd(dppf)Cl₂·CH₂Cl₂ (82.93 mg, 101.56 umol, 0.1 eq), AcOK (299.00 mg, 3.05 mmol, 3 eq) the mixture was bubbled with N2 for 1 minutes, and stirred at 110° C. for 3 h. LCMS showed the starting material was consumed completely and desired MS was detected. The reaction mixture was poured into water (50 mL). The aqueous phase was extracted with ethyl acetate (100 mL*2). The combined organic phase was dried with anhydrous Na₂SO₄, filtered and concentrated in vacuum. Compound 2-[(6-borono-3-morpholinosulfonyl-4-quinolyl)amino]benzoic acid (900 mg, crude) was obtained as a black oil. MS (M+H)⁺=458.1.

Step 6. Synthesis of 2-[[3-morpholinosulfonyl-6-(1H-pyrrolo[2,3-c]pyridin-4-yl)-4-quinolyl]amino]benzoic acid (160A): To a stirred solution of 2-[(6-borono-3-morpholinosulfonyl-4-quinolyl)amino]benzoic acid (40 mg, 87.48 umol, 1 eq) in DMF (1 mL) and H₂O (0.1 mL) was added 4-bromo-1H-pyrrolo[2,3-c]pyridine (17.24 mg, 87.48 umol, 1 eq), Cs₂CO₃ (85.50 mg, 262.43 umol, 3 eq), Pd(dppf)Cl₂ (6.40 mg, 8.75 umol, 0.1 eq) the mixture was bubbled with N₂ for 1 minute, and stirred at 100° C. for 2 h. LCMS showed the starting material was consumed completely and desired MS was detected. The reaction mixture was filtered, and filtrate was purified directly. The residue was purified by prep-HPLC (column: Welch Xtimate C18 100*25 mm*3 um; mobile phase: [water(0.05% HCl)-ACN]; B %: 5%-35%,8 min). Compound 2-[[3-morpholinosulfonyl-6-(1H-pyrrolo[2,3-c]pyridin-4-yl)-4-quinolyl]amino]benzoic acid (5.40 mg, 9.33 umol, 10.67% yield, 97.81% purity, HCl) was obtained as a yellow solid. ¹H NMR (400 MHz, DMSO-d6) δ=10.47-10.34 (m, 1H), 9.16 (d, J=1.2 Hz, 2H), 8.36-8.27 (m, 3H), 8.23 (t, J=2.9 Hz, 1H), 8.11 (d, J=0.9 Hz, 1H), 8.01 (dd, J=1.4, 7.9 Hz, 1H), 7.46 (t, J=7.8 Hz, 1H), 7.15 (br t, J=7.6 Hz, 1H), 6.94-6.83 (m, 1H), 6.26 (s, 1H), 3.46-3.35 (m, 4H), 3.10 (br d, J=8.8 Hz, 4H). MS (M/2+H)⁺=265.7.

### Example 13—Synthesis of Compound 161A

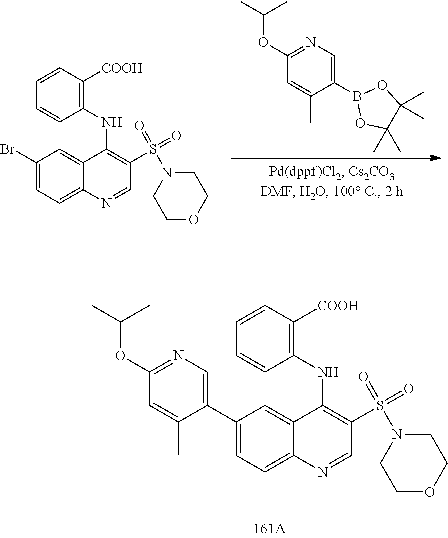 [SMILES: O=C(O)c1ccccc1Nc1c(S(=O)(=O)N2CCOCC2)cnc2ccc(Br)cc12 ; Cc1cc(OC(C)C)ncc1B1OC(C)(C)C(C)(C)O1 ; Cc1cc(OC(C)C)ncc1-c1ccc2ncc(S(=O)(=O)N3CCOCC3)c(Nc3ccccc3C(=O)O)c2c1]

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and 2-isopropoxy-4-methyl-5-(4,4,5,5-tetramethyl-1,3,2-dioxaborolan-2-yl)pyridine (22.52 mg, 81.24 umol, 1 eq), was bubbled with N₂ for 1 minutes, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 20%-50%,8 min). Compound 2-[[6-(6-isopropoxy-4-methyl-3-pyridyl)-3-morpholinosulfonyl-4-quinolyl]amino] benzoic acid (5.70 mg, 9.07 umol, 11.17% yield, 95.37% purity, HCl) was obtained as a yellow solid. ¹H NMR (400 MHz, DMSO-d₆) δ ppm=10.61 (br s. 1H), 9.16 (s, 1H), 8.23 (br d, J=8.75 Hz, 1H), 7.98 (br d, J=4.25 Hz, 2H), 7.66 (s, 1H), 7.54 (s, 1H), 7.45 (br t, J=7.69 Hz, 1H), 7.17 (br t, J=7.57 Hz, 1H), 6.98 (br d, J=8.13 Hz, 1H), 6.67 (s, 1H), 5.14-5.28 (m, 1H), 3.52 (br s, 2H), 3.43 (br s, 2H), 3.12 (br s, 4H), 1.97 (s, 3H), 1.20-1.36 (m, 6H). MS (M+H)⁺=563.2

### Example 14—Synthesis of Compound 162A

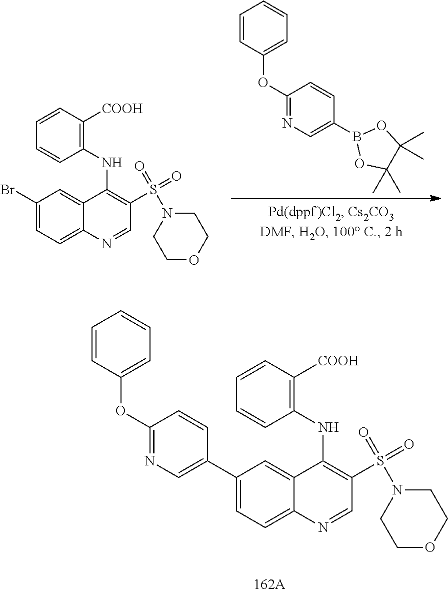 [SMILES: O=C(O)c1ccccc1Nc1c(S(=O)(=O)N2CCOCC2)cnc2ccc(Br)cc12 ; CC1(C)OB(c2ccc(Oc3ccccc3)nc2)OC1(C)C ; O=C(O)c1ccccc1Nc1c(S(=O)(=O)N2CCOCC2)cnc2ccc(-c3ccc(Oc4ccccc4)nc3)cc12]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and 2-phenoxy-5-(4,4,5,5-tetramethyl-1,3,2-dioxaborolan-2-yl) pyridine (21.12 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase:[water(0.05% HCl)-ACN]; B %: 20%-50%,8 min). Compound 2-[[3-morpholinosulfonyl-6-(6-phenoxy-3-pyridyl)-4-quinolyl]amino]benzoic acid (4.80 mg, 7.69 umol, 10.82% yield, 99.18% purity, HCl) was obtained as a yellow solid. ¹H NMR (400 MHz, DMSO-d6) δ ppm=10.61 (br s, 1H), 9.10 (s, 1H), 8.18-8.25 (m, 2H), 8.08 (d, J=2.25 Hz, 1H), 8.03 (dd, J=7.88, 1.50 Hz, 1H), 7.76-7.87 (m, 2H), 7.33-7.47 (m, 3H), 7.19-7.28 (m, 1H), 7.11-7.17 (m, 3H), 7.07 (d, J=8.50 Hz, 1H), 6.83 (d, J=8.25 Hz, 1H), 3.51 (br dd, J=5.82, 3.31 Hz, 2H), 3.37-3.45 (m, 2H), 3.01-3.17 (m, 4H). MS (M+H)⁺=583.2

### Example 15—Synthesis of Compound 163A

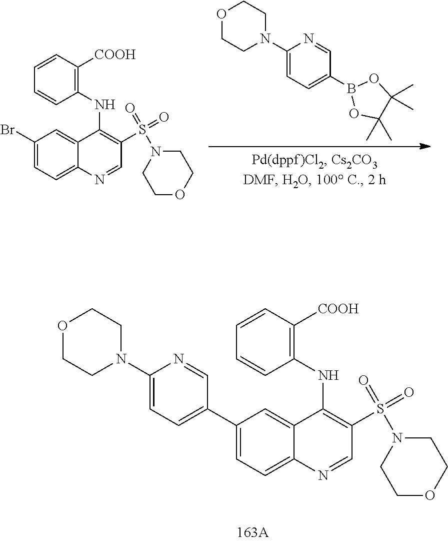 [SMILES: O=C(O)c1ccccc1Nc1c(S(=O)(=O)N2CCOCC2)cnc2ccc(Br)cc12 ; CC1(C)OB(c2ccc(N3CCOCC3)nc2)OC1(C)C ; O=C(O)c1ccccc1Nc1c(S(=O)(=O)N2CCOCC2)cnc2ccc(-c3ccc(N4CCOCC4)nc3)cc12]

To a solution of 2-[(6-bromo-3-morpholinosulfonyl-4-quinolyl)amino]benzoic acid (40 mg. 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and 4-[5-(4,4,5,5-tetramethyl-1,3,2-dioxaborolan-2-yl)-2-pyridyl]morpholine (23.57 mg, 81.24 umol, 1 eq), was bubbled with N₂ for 1 minute, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min). Compound 2-[[6-(6-morpholino-3-pyridyl)-3-morpholinosulfonyl-4-quinolyl]amino]benzoic acid (15.40 mg, 24.60 umol, 30.28% yield, 97.78% purity, HCl) was obtained as yellow solid. ¹H NMR (400 MHz, DMSO-d6) δ ppm 10.80 (br s, 1H), 9.10 (s, 1H), 8.24-8.32 (m, 2H), 8.06 (d, J=7.70 Hz, 1H), 8.01 (d, J=2.20 Hz, 1H), 7.72 (s, 1H), 7.68 (br d, J=9.17 Hz, 1H), 7.44 (t, J=7.64 Hz, 1H), 7.27 (t, J=7.46 Hz, 1H), 7.15 (br d, J=8.80 Hz, 1H), 7.05 (br d, J=8.07 Hz, 1H), 3.70 (br d, J=4.65 Hz, 4H), 3.52-3.70 (m, 6H), 3.42-3.52 (m, 2H), 3.08-3.24 (m, 4H). MS (M+H)⁺=576.1.

### Example 16—Synthesis of Compound 164A

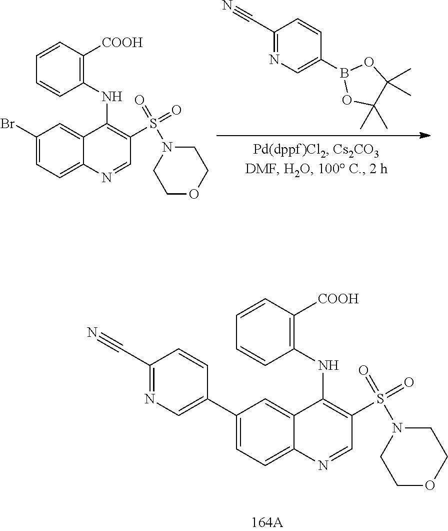 [SMILES: O=C(O)c1ccccc1Nc1c(S(=O)(=O)N2CCOCC2)cnc2ccc(Br)cc12 ; CC1(C)OB(c2ccc(C#N)nc2)OC1(C)C ; N#Cc1ccc(-c2ccc3ncc(S(=O)(=O)N4CCOCC4)c(Nc4ccccc4C(=O)O)c3c2)cn1]

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and 5-(4,4,5,5-tetramethyl-1,3,2-dioxaborolan-2-yl)pyridine-2-carbonitrile (18.69 mg, 81.24 umol, 1 eq), was bubbled with N₂ for 1 minutes, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water (0.05% HCl)-ACN]; B %: 15%-40%,8 min) Compound 2-[[6-(6-cyano-3-pyridyl)-3-morpholinosulfonyl-4-quinolyl]amino]benzoic acid (5.20 mg, 9.00 umol, 11.08% yield, 95.58% purity, HCl) was obtained as yellow solid. (10.20 mg, 16.61 umol, 20.44% yield, 97.05% purity, HCl) was obtained as yellow solid. ¹H NMR (400 MHz, DMSO-d₆) δ ppm=10.70 (br s, 1H), 9.13 (s, 1H), 8.59 (d, J=1.59 Hz, 1H), 8.31-8.36 (m, 1H), 8.23-8.28 (m, 1H), 8.08 (d, J=2.08 Hz, 1H), 8.06 (d, J=1.71 Hz, 1H), 8.04 (d, J=1.34 Hz, 1H), 7.95 (d, J=1.59 Hz, 1H), 7.33-7.44 (m, 1H), 7.18 (t, J=7.58 Hz, 1H), 6.91 (d, J=8.19 Hz, 1H), 3.49-3.62 (m, 2H), 3.39-3.48 (m, 2H), 3.02-3.21 (m, 4H). MS (M+H)⁺=516.0.

### Example 17—Synthesis of Compound 165A

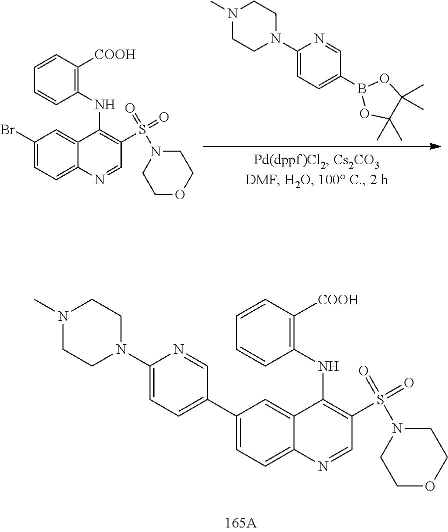

To a solution of 2-[(6-bromo-3-morpholinosulfonyl-4-quinolyl)amino] benzoic acid (40 mg, 81.24 umol, 1 eq) in H₂O (0.1 mL) and DMF (0.5 mL) was added Cs₂CO₃ (79.41 mg, 243.73 u mol, 3 eq), Pd(dppf)Cl₂ (5.94 mg, 8.12 umol, 0.1 eq) and 1-methyl-4-[5-(4,4,5,5-tetramethyl-1,3,2-dioxaborolan-2-yl)-2-pyridyl]piperazine (24.63 mg, 81.24 umol, 1 eq), was bubbled with N₂ for 1 minutes, the mixture was stirred at 100° C. for 2 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min) Compound 2-[[6-[6-(4-methylpiperazin-1-yl)-3-pyridyl]-3-morpholinosulfonyl-4-quinolyl]amino]benzoic acid (28.70 mg, 48.75 umol, 60.01% yield, 100% purity) was obtained as a yellow solid. ¹H NMR (400 MHz, DMSO-d₆) δ ppm=9.05 (s, 1H), 8.17-8.23 (m, 1H), 8.12-8.17 (m, 1H), 8.05 (br d, J=2.50 Hz, 2H), 7.66 (s, 1H), 7.57 (dd, J=8.94, 2.31 Hz, 1H), 7.40 (br t, J=7.00 Hz, 1H), 7.21 (t, J=7.57 Hz, 1H), 6.97 (br d, J=9.01 Hz, 1H), 6.87 (d, J=8.25 Hz, 1H), 4.39 (br d, J=11.76 Hz, 2H), 3.34-3.58 (m, 6H), 2.94-3.23 (m, 8H), 2.80 (s, 3H). MS (M+H)⁺=589.2

### Example 18—Synthesis of Compound 166A

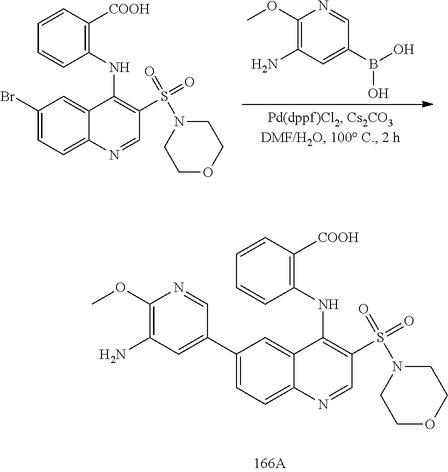 [SMILES: O=C(O)c1ccccc1Nc1c(S(=O)(=O)N2CCOCC2)cnc2ccc(Br)cc12 ; COc1ncc(B(O)O)cc1N ; COc1ncc(-c2ccc3ncc(S(=O)(=O)N4CCOCC4)c(Nc4ccccc4C(=O)O)c3c2)cc1N]

To a stirred solution of 2-[(6-bromo-3-morpholinosulfonyl-4-quinolyl)amino]benzoic acid (40 mg, 81.24 umol, 1 eq) in DMF (0.5 mL) and H₂O (0.1 mL) was added (5-amino-6-methoxy-3-pyridyl)boronic acid (13.65 mg, 81.24 umol, 1 eq), Cs₂CO₃(79.41 mg, 243.73 umol, 3 eq), Pd(dppf)Cl₂ (5.94 mg, 8.12 umol, 0.1 eq) the mixture was bubbled with N₂ for 1 minute, and stirred at 100° C. for 2 h. LCMS showed the starting material was consumed completely and desired MS was detected. The reaction mixture was filtered, the filtrate was purified by prep-HPLC (column: Welch Xtimate C18 100*25 mm*3 um; mobile phase: [water(0.05% HCl)-ACN]; B %: 15%-45%,8 min). Afford 15 mg crude. The crude was purified by prep-HPLC (column: Phenomenex Gemini NX-C18(75*30 mm*3 um); mobile phase: [water(10 mM NH₄HCO₃)-ACN]; B %: 1%-24%,10 min). Compound 2-[[6-(5-amino-6-methoxy-3-pyridyl)-3-morpholinosulfonyl-4-quinolyl]amino]benzoic acid (8.20 mg, 15.04 umol, 18.51% yield, 98.23% purity) was obtained as a yellow solid. ¹H NMR (400 MHz, DMSO-d₆+TFA) δ=9.28 (s, 1H), 8.21 (s, 2H), 8.11 (dd, J=1.4, 7.9 Hz, 1H), 7.70 (s, 1H), 7.53 (d, J=2.1 Hz, 1H), 7.51-7.48 (m, 1H), 7.42-7.37 (m, 1H), 7.34 (d, J=2.3 Hz, 1H), 7.28 (d, J=7.9 Hz, 1H), 3.94 (s, 3H), 3.65-3.57 (m, 2H), 3.56-3.49 (m, 2H), 3.27-3.17 (m, 4H). MS (M+H)⁺=536.2.

### Example 19—Synthesis of Compound 167A

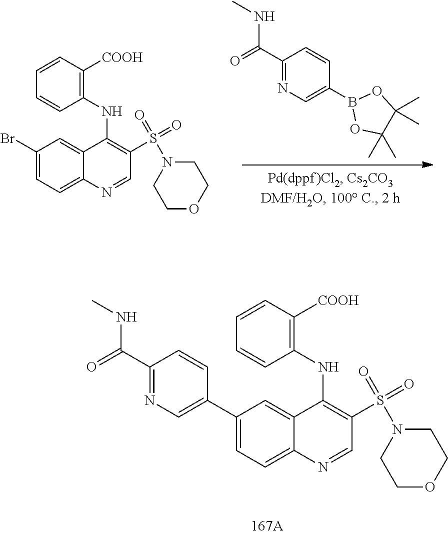

To a stirred solution of 2-[(6-bromo-3-morpholinosulfonyl-4-quinolyl)amino]benzoic acid (40 mg, 81.24 umol, 1 eq) in DMF (0.5 mL) and H₂O (0.1 mL) was added N-methyl-5-(4,4,5,5-tetramethyl-1,3,2-dioxaborolan-2-yl)pyridine-2-carboxamide (21.30 mg, 81.24 umol, 1 eq), Cs₂CO₃ (79.41 mg, 243.73 umol, 3 eq), Pd(dppf)Cl₂ (5.94 mg, 8.12 umol, 0.1 eq), the mixture was bubbled with N₂ for 1 minute, and stirred at 100° C. for 2 h. LCMS showed the starting material was consumed completely and desired MS was detected. The reaction mixture was filtered, The filtrate was purified by prep-HPLC (column: Welch Xtimate C18 100*25 mm*3 um; mobile phase: [water(0.05% HCl)-ACN]; B %: 15%-45%,8 min). Compound 2-[[6-[6-(methylcarbamoyl)-3-pyridyl]-3-morpholinosulfonyl-4-quinolyl]amino]benzoic acid (30.10 mg, 49.89 umol, 61.41% yield, 96.81% purity, HCl) was obtained as a yellow solid. ¹H NMR (400 MHz, DMSO-d₆+D₂O) 5=9.12 (s, 1H), 8.37 (d, J=1.8 Hz, 1H), 8.30 (dd, J=1.9, 8.9 Hz, 1H), 8.21 (d, J=8.8 Hz, 1H), 8.06 (dd, J=1.5, 7.9 Hz, 1H), 8.02-7.98 (m, 1H), 7.95-7.90 (m, 1H), 7.83 (d, J=1.8 Hz, 1H), 7.50-7.40 (m, 1H), 7.31-7.24 (m, 1H), 7.01 (d, J=8.1 Hz, 1H), 3.59-3.50 (m, 2H), 3.49-3.40 (m, 2H), 3.20-3.05 (m, 4H), 2.79 (s, 3H). MS (M+H)⁺=548.3.

### Example 20—Synthesis of Compound 168A

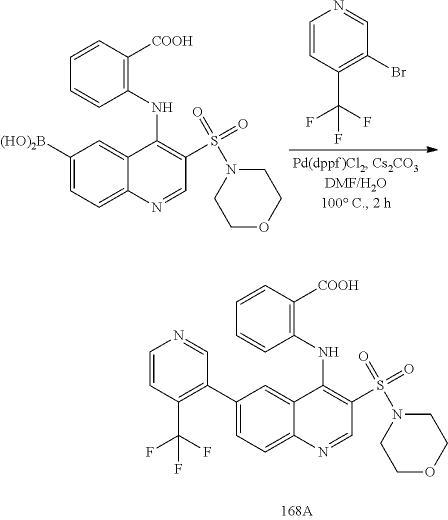 [SMILES: O=C(O)c1ccccc1Nc1c(S(=O)(=O)N2CCOCC2)cnc2ccc(B(O)O)cc12 ; FC(F)(F)c1ccncc1Br ; O=C(O)c1ccccc1Nc1c(S(=O)(=O)N2CCOCC2)cnc2ccc(-c3cnccc3C(F)(F)F)cc12]

To a stirred solution of 2-[(6-borono-3-morpholinosulfonyl-4-quinolyl)amino]benzoic acid (40 mg, 87.48 umol, 1 eq) in DMF (1 mL) and H₂O (0.2 mL) was added 3-bromo-4-(trifluoromethyl)pyridine (19.77 mg, 87.48 umol, 1 eq), Cs₂CO₃ (28.50 mg, 87.48 umol, 1 eq), Pd(dppf)Cl₂ (64.01 mg, 87.48 umol, 1 eq) the mixture was bubbled with N₂ for 1 minute, and stirred at 100° C. for 2 h. LCMS showed the starting material was consumed completely and desired MS was detected. The reaction mixture was filtered, and filtrate was purified directly. The filtrate was purified by prep-HPLC (column: Welch Xtimate C18 100*25 mm*3 um; mobile phase: [water(0.05% HCl)ACN]; B %: 15%-35%,8 min). Compound 2-[[3-morpholinosulfonyl-6-[4-(trifluoromethyl)-3-pyridyl]-4-quinolyl]amino]benzoic acid (5.00 mg, 8.26 umol, 9.44% yield, 98.28% purity, HCl) was obtained as a yellow solid. ¹H NMR (400 MHz, DMSO-d₆) δ=10.66-10.52 (m, 1H), 9.18 (d, J=1.6 Hz, 1H), 8.86 (d, J=5.1 Hz, 1H), 8.44 (d, J=3.4 Hz, 1H), 8.31-8.20 (m, 1H), 7.92 (br d, J=7.7 Hz, 2H), 7.82 (d, J=5.3 Hz, 1H), 7.62 (s, 1H), 7.42-7.31 (m, 1H), 7.07 (br d, J=7.5 Hz, 1H), 6.95-6.79 (m, 1H), 3.56-3.47 (m, 2H), 3.39 (br d, J=5.5 Hz, 2H), 3.20-3.01 (m, 4H). MS (M+H)=559.3.

### Example 21—Synthesis of Compound 169A

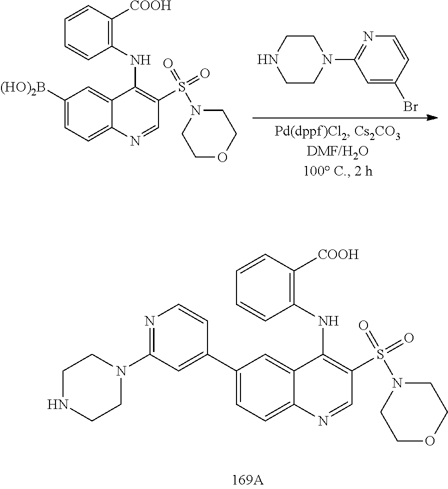 [SMILES: O=C(O)c1ccccc1Nc1c(S(=O)(=O)N2CCOCC2)cnc2ccc(B(O)O)cc12 ; Brc1ccnc(N2CCNCC2)c1 ; O=C(O)c1ccccc1Nc1c(S(=O)(=O)N2CCOCC2)cnc2ccc(-c3ccnc(N4CCNCC4)c3)cc12]

To a stirred solution of 2-[(6-borono-3-morpholinosulfonyl-4-quinolyl)amino]benzoic acid (40 mg, 87.48 umol, 1 eq) in DMF (1 mL) and H₂O (0.2 mL) was added 1-(4-bromo-2-pyridyl)piperazine (21.18 mg, 87.48 umol, 1 eq), Cs₂CO₃ (28.50 mg, 87.48 umol, 1 eq), Pd(dppf)Cl₂ (64.01 mg, 87.48 umol, 1 eq) the mixture was bubbled with N₂ for 1 minute, and stirred at 100° C. for 2 h. LCMS showed the starting material was consumed completely and desired MS was detected. The reaction mixture was filtered, and filtrate was purified directly, The filtrate was purified by prep-HPLC (column: Welch Xtimate C18 100*25 mm*3 um; mobile phase: [water(0.05% HCl)-ACN]; B %: 5%-35%,8 min). Compound 2-[[3-morpholinosulfonyl-6-(2-piperazin-1-yl-4-pyridyl)-4-quinolyl]amino]benzoic acid (5.10 mg, 8.35 umol, 9.54% yield, 100% purity, HCl) was obtained as a yellow solid. 1H NMR (400 MHz, DMSO-d₆+D₂O) δ=9.04 (s, 1H), 8.20 (d, J=1.7 Hz, 1H), 8.18-8.14 (m, 1H), 8.09-8.03 (m, 2H), 7.82 (d, J=1.5 Hz, 1H), 7.44-7.36 (m, 1H), 7.20 (s, 1H), 6.86 (d, J=8.3 Hz, 1H), 6.76 (d, J=5.7 Hz, 1H), 6.67 (s, 1H), 3.70-3.58 (m, 4H), 3.55-3.47 (m, 2H), 3.44-3.37 (m, 2H), 3.20 (br t, J=5.0 Hz, 4H), 3.07 (br dd, J=5.6, 18.5 Hz, 4H). MS (M+H)⁺=575.2.

### Example 22—Synthesis of Compound 170A

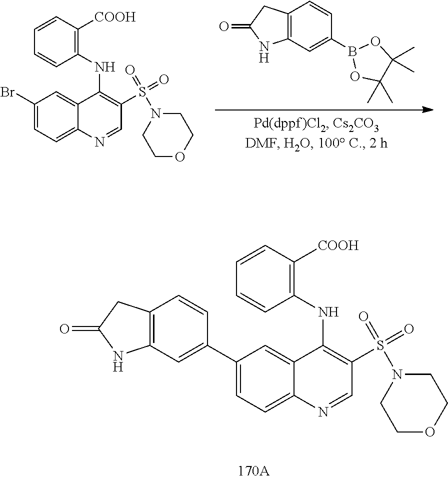 [SMILES: O=C(O)c1ccccc1Nc1c(S(=O)(=O)N2CCOCC2)cnc2ccc(Br)cc12 ; CC1(C)OB(c2ccc3c(c2)NC(=O)C3)OC1(C)C ; O=C1Cc2ccc(-c3ccc4ncc(S(=O)(=O)N5CCOCC5)c(Nc5ccccc5C(=O)O)c4c3)cc2N1]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and 6-(4,4,5,5-tetramethyl-1,3,2-dioxaborolan-2-yl)indolin-2-one (18.42 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min)Afford crude product (18 mg). The crude product was purified by prep-HPLC (column: Welch Xtimate C18 100*25 mm*3 um; mobile phase: [water(0.05% HCl)-ACN]; B %: 10%-30%,8 min) Compound 2-[[3-morpholinosulfonyl-6-(2-oxoindolin-6-yl)-4-quinolyl]amino]benzoic acid (1.60 mg, 2.75 umol, 3.87% yield, 100% purity, HCl) was obtained as yellow solid. 1H NMR (400 MHz, DMSO-d₆) δ ppm=10.54 (s, 2H), 9.11 (s, 1H), 8.14-8.23 (m, 2H), 8.06 (dd, J=7.95, 1.59 Hz, 1H), 7.74 (s, 1H), 7.42 (t, J=6.91 Hz, 1H), 7.23 (d, J=7.82 Hz, 1H), 7.18 (t, J=7.64 Hz, 1H), 6.86 (dd, J=13.88, 7.89 Hz, 2H), 6.78 (s, 1H), 3.55 (br d, J=3.79 Hz, 2H), 3.51 (s, 2H), 3.42-3.47 (m, 2H), 3.04-3.19 (m, 4H). MS (M+H)⁺=545.1

### Example 23—Synthesis of Compound 171A

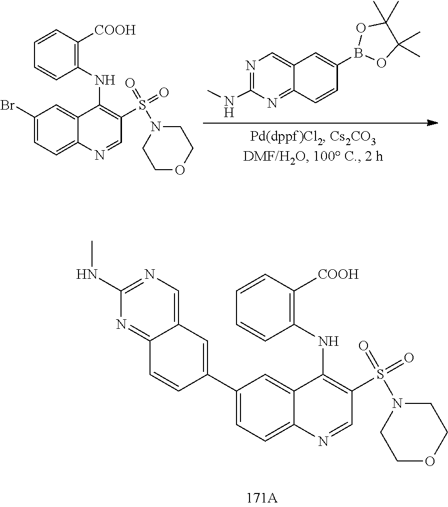

To a stirred solution of 2-[(6-bromo-3-morpholinosulfonyl-4-quinolyl)amino]benzoic acid (40 mg, 81.24 umol, 1 eq) in DMF (0.5 mL) and H₂O (0.1 mL) was added N-methyl-6-(4,4,5,5-tetramethyl-1,3,2-dioxaborolan-2-yl)quinazolin-2-amine (23.17 mg, 81.24 umol, 1 eq), Cs₂CO₃ (79.41 mg, 243.73 umol, 3 eq), Pd(dppf)Cl₂ (5.94 mg, 8.12 umol, 0.1 eq) the mixture was bubbled with N₂ for 1 minute, and stirred at 100° C. for 2 h. LCMS showed the starting material was consumed completely and desired MS was detected. The reaction mixture was filtered, the filtrate was purified by prep-HPLC (column: Welch Xtimate C18 100*25 mm*3 um; mobile phase: [water(0.05% HCl)-ACN]; B %: 15%-45%,8 min). Compound 2-[[6-[2-(methylamino)quinazolin-6-yl]-3-morpholinosulfonyl-4-quinolyl]amino]benzoic acid (1.40 mg, 2.31 umol, 2.84% yield, 100% purity, HCl) was obtained as a yellow Solid. ¹H NMR (400 MHz, DMSO-d₆) δ=10.73-10.57 (m, 1H), 9.22 (br d, J=4.6 Hz, 1H), 9.10 (s, 1H), 8.32-8.27 (m, 1H), 8.26-8.22 (m, 1H), 8.07 (dd, J=1.5, 7.9 Hz, 1H), 7.93 (br s, 1H), 7.86-7.75 (m, 2H), 7.48-7.37 (m, 1H), 7.21 (t, J=7.5 Hz, 1H), 6.89 (d, J=8.3 Hz, 1H), 3.44 (br s, 4H), 3.05 (br s, 7H). MS (M+H)⁺=571.3.

### Example 24—Synthesis of Compound 172A

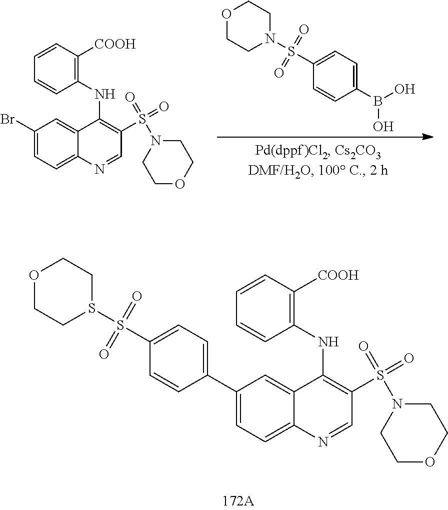

To a stirred solution of 2-[(6-bromo-3-morpholinosulfonyl-4-quinolyl)amino]benzoic acid (40 mg, 81.24 umol, 1 eq) in DMF (0.5 mL) and H₂O (0.1 mL) was added (4-morpholinosulfonylphenyl)boronic acid (22.03 mg, 81.24 umol, 1 eq), Cs₂CO₃ (79.41 mg, 243.73 umol, 3 eq), Pd(dppf)Cl₂ (5.94 mg, 8.12 umol, 0.1 eq) the mixture was bubbled with N₂ for 1 minute, and stirred at 100° C. for 2 h. LCMS showed the starting material was consumed completely and desired MS was detected. The reaction mixture was filtered, the filtrate was purified by prep-HPLC (column: Welch Xtimate C18 100*25 mm*3 um; mobile phase: [water(0.05% HCl)-ACN]; B %: 15%-45%,8 min). Compound 2-[[3-morpholinosulfonyl-6-(4-morpholinosulfonylphenyl)-4-quinolyl]amino]benzoic acid (22.20 mg, 32.88 umol, 40.47% yield, 100% purity, HCl) was obtained as a yellow solid. ¹H NMR (400 MHz, DMSO-d₆+D₂O) δ=9.08 (s, 1H), 8.28-8.23 (m, 1H), 8.22-8.18 (m, 1H), 8.05 (dd, J=1.5, 7.9 Hz, 1H), 7.85 (d, J=1.8 Hz, 1H), 7.73 (d, J=8.4 Hz, 2H), 7.56 (d, J=8.4 Hz, 2H), 7.44-7.36 (m, 1H), 7.19 (t, J=7.4 Hz, 1H), 6.84 (d, J=8.1 Hz, 1H), 3.64-3.58 (m, 4H), 3.55-3.47 (m, 2H), 3.44-3.35 (m, 2H), 3.16-3.01 (m, 4H), 2.90-2.81 (m, 4H). MS (M+H)⁺=639.2.

### Example 25—Synthesis of Compound 173A

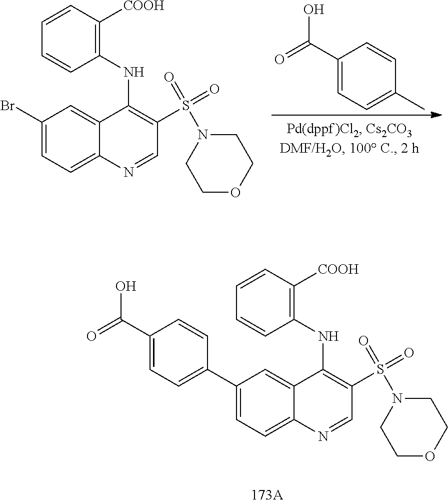

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added CS₂CO₃ (79.41 mg, 243.73 umol, 3 eq), Pd(dppf)Cl₂ (5.94 mg, 8.12 umol, 0.1 eq) and 4-boronobenzoic acid (13.48 mg, 81.24 umol, 1 eq) was bubbled with N₂ for 1 minutes, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 15%-45%,8 min) Compound 2-[[6-(4-carboxyphenyl)-3-morpholinosulfonyl-4-quinolyl]amino]benzoic acid (15.9 mg, 27.17 umol, 33.45% yield, 97.42% purity, HCl) was obtained as a yellow solid. ¹H NMR (400 MHz, DMSO-d₆+D₂O) δ ppm=9.08 (s, 1H), 8.22-8.27 (m, 1H), 8.14-8.20 (m, 1H), 8.06 (dd, J=8.00, 1.63 Hz, 1H), 7.92 (d, J=8.50 Hz, 2H), 7.79 (d, J=1.88 Hz, 1H), 7.38-7.45 (m, 3H), 7.22 (t, J=7.57 Hz, 1H), 6.90 (d, J=8.13 Hz, 1H), 3.49-3.58 (m, 2H), 3.38-3.46 (m, 2H), 3.01-3.17 (m, 4H). MS (M+H)⁺=534.1

### Example 26—Synthesis of Compound 174A

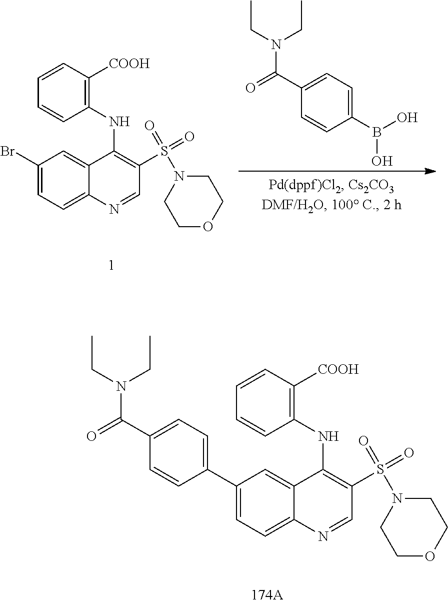 [SMILES: O=C(O)c1ccccc1Nc1c(S(=O)(=O)N2CCOCC2)cnc2ccc(Br)cc12 ; CCN(CC)C(=O)c1ccc(B(O)O)cc1 ; CCN(CC)C(=O)c1ccc(-c2ccc3ncc(S(=O)(=O)N4CCOCC4)c(Nc4ccccc4C(=O)O)c3c2)cc1]

To a stirred solution of 2-[(6-bromo-3-morpholinosulfonyl-4-quinolyl)amino]benzoic acid (40 mg, 81.24 umol, 1 eq) in DMF (0.5 mL) and H₂O (0.1 mL) was added [4-(diethylcarbamoyl)phenyl]boronic acid (17.96 mg, 81.24 umol, 1 eq), Cs₂CO₃ (79.41 mg, 243.73 umol, 3 eq), Pd(dppf)Cl₂ (5.94 mg, 8.12 umol, 0.1 eq) the mixture was bubbled with N₂ for 1 minute, and stirred at 100° C. for 2 h. LCMS showed the starting material was consumed completely and desired MS was detected. The reaction mixture was filtered, the filtrate was purified by prep-HPLC (column: Welch Xtimate C18 100*25 mm*3 um; mobile phase: [water (0.05% HCl)-ACN]; B %: 15%-45%,8 min). Compound 2-[[6-[4-(diethylcarbamoyl)phenyl]-3-morpholinosulfonyl-4-quinolyl]amino]benzoic acid (15.70 mg, 25.11 umol, 30.91% yield, 100% purity, HCl) was obtained as a yellow solid. ¹H NMR (400 MHz, DMSO-d6+TFA) δ=9.20 (s, 1H), 8.31 (dd, J=1.7, 8.9 Hz, 1H), 8.17 (d, J=8.8 Hz, 1H), 8.11 (dd, J=1.3, 7.8 Hz, 1H), 7.71 (d, J=1.6 Hz, 1H), 7.56-7.51 (m, 1H), 7.49-7.42 (m, 1H), 7.37 (d, J=7.8 Hz, 1H), 7.30 (d, J=8.2 Hz, 2H), 7.19 (d, J=8.3 Hz, 2H), 3.65-3.50 (m, 4H), 3.39 (br d, J=4.5 Hz, 2H), 3.30-3.07 (m, 6H), 1.18-0.91 (m, 6H). MS (M+H)⁺=589.3.

### Example 27—Synthesis of Compound 175A

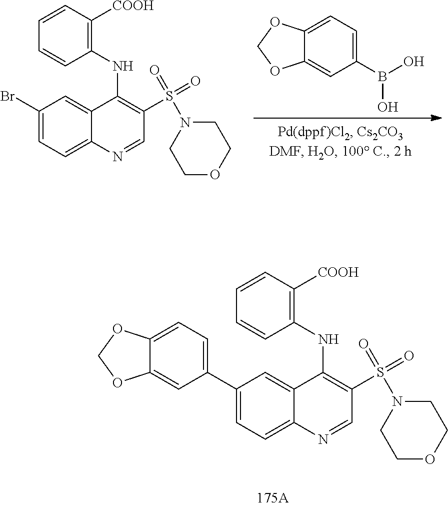 [SMILES: O=C(O)c1ccccc1Nc1c(S(=O)(=O)N2CCOCC2)cnc2ccc(Br)cc12 ; OB(O)c1ccc2c(c1)OCO2 ; O=C(O)c1ccccc1Nc1c(S(=O)(=O)N2CCOCC2)cnc2ccc(-c3ccc4c(c3)OCO4)cc12]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and 1,3-benzodioxol-5-ylboronic acid (11.80 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 15%-40%,8 min). Compound 2-[[6-(1,3-benzodioxol-5-yl)-3-morpholinosulfonyl-4-quinolyl]amino]benzoic acid (9.00 mg, 15.68 umol, 22.06% yield, 99.33% purity, HCl) was obtained as a yellow solid. ¹H NMR (400 MHz, DMSO-d₆) δ ppm=10.62 (br s, 1H), 9.09 (s, 1H), 8.14-8.21 (m, 2H), 8.06 (dd, J=7.88, 1.50 Hz, 1H), 7.68 (d, J=1.38 Hz, 1H), 7.41-7.47 (m, 1H), 7.20 (t, J=7.44 Hz, 1H), 6.90-6.97 (m, 2H), 6.81-6.85 (m, 1H), 6.79 (d, J=1.75 Hz, 1H), 6.05 (d, J=3.00 Hz, 2H), 3.51-3.59 (m, 2H), 3.41-3.49 (m, 2H), 3.05-3.19 (m, 4H). MS (M+H)⁺=534.0.

### Example 28—Synthesis of Compound 176A

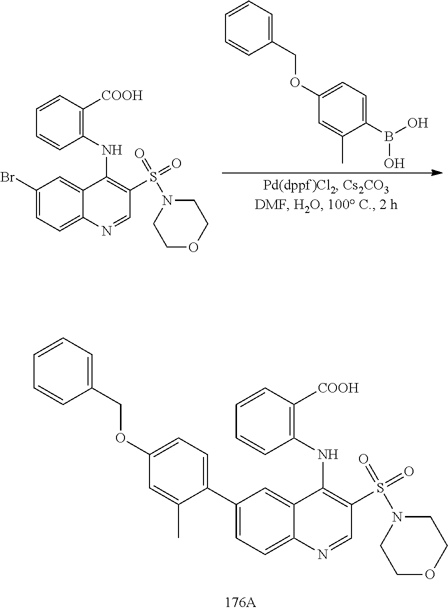 [SMILES: O=C(O)c1ccccc1Nc1c(S(=O)(=O)N2CCOCC2)cnc2ccc(Br)cc12 ; Cc1cc(OCc2ccccc2)ccc1B(O)O ; Cc1cc(OCc2ccccc2)ccc1-c1ccc2ncc(S(=O)(=O)N3CCOCC3)c(Nc3ccccc3C(=O)O)c2c1]

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and (4-benzyloxy-2-methyl-phenyl)boronic acid (19.67 mg, 81.24 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 30%-50%,8 min). Compound 2-[[6-(4-benzyloxy-2-methyl-phenyl)-3-morpholinosulfonyl-4-quinolyl]amino]benzoic acid (9.10 mg, 14.04 umol, 17.28% yield, 99.66% purity, HCl) was obtained as a yellow solid. ¹H NMR (400 MHz, DMSO-d₆+D₂O) δ ppm=9.09 (s, 1H), 8.13 (d, J=8.63 Hz, 1H), 7.96 (dd, J=7.88, 1.50 Hz, 1H), 7.87 (dd, J=8.69, 1.81 Hz, 1H), 7.46 (d, 0.1=1.75 Hz, 1H), 7.36-7.43 (m, 5H), 7.29-7.34 (m, 1H), 7.11 (t, J=7.25 Hz, 1H), 6.87-6.89 (m, 1H), 6.85 (s, 1H), 6.78-6.84 (m, 2H), 5.06 (s, 2H), 3.45-3.53 (m, 2H), 3.32-3.42 (m, 2H), 2.99-3.14 (m, 4H), 1.96 (s, 3H). MS (M+H)⁺=610.2

### Example 29—Synthesis of Compound 177A

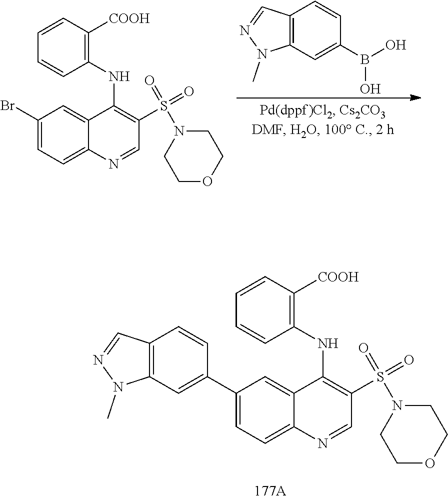 [SMILES: O=C(O)c1ccccc1Nc1c(S(=O)(=O)N2CCOCC2)cnc2ccc(Br)cc12 ; Cn1ncc2ccc(B(O)O)cc21 ; Cn1ncc2ccc(-c3ccc4ncc(S(=O)(=O)N5CCOCC5)c(Nc5ccccc5C(=O)O)c4c3)cc21]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and (1-methylindazol-6-yl)boronic acid (12.51 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The mixture was purified by prep-HPLC (column: Welch Xtimate C18 100*25 mm*3 um; mobile phase: [water(0.05% HCl)-ACN]; B %: 10%-40%,8 min) Compound 2-[[6-(1-methylindazol-6-yl)-3-morpholinosulfonyl-4-quinolyl]amino]benzoic acid (17.10 mg, 29.48 umol, 41.47% yield, 100% purity, HCl) was obtained as a yellow solid. ¹H NMR (400 MHz, DMSO-d₆) δ ppm=10.72 (br s, 1H), 9.12 (s, 1H), 8.36 (dd, J=8.80, 1.59 Hz, 1H), 8.25 (d, J=8.68 Hz, 1H), 8.10 (dd, J=7.89, 1.28 Hz, 1H), 8.05 (s, 1H), 7.85 (d, J=1.71 Hz, 1H), 7.76 (d, J=8.31 Hz, 1H), 7.53 (t, J=7.64 Hz, 1H), 7.41 (s, 1H), 7.29 (t, J=7.64 Hz, 1H), 7.07 (br d, J=8.44 Hz, 2H), 3.54-3.62 (m, 2H), 3.44-3.52 (m, 2H), 3.06-3.25 (m, 4H). MS (M+H)⁺=544.1.

### Example 30—Synthesis of Compound 178A

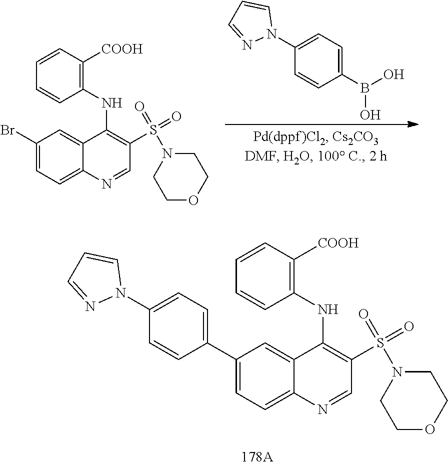 [SMILES: O=C(O)c1ccccc1Nc1c(S(=O)(=O)N2CCOCC2)cnc2ccc(Br)cc12 ; OB(O)c1ccc(-n2cccn2)cc1 ; O=C(O)c1ccccc1Nc1c(S(=O)(=O)N2CCOCC2)cnc2ccc(-c3ccc(-n4cccn4)cc3)cc12]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and (4-pyrazol-1-ylphenyl) boronic acid (13.36 mg, 71.09 umol, 1 eq), was bubbled with N₂ for 1 minute, the mixture was stirred at 100° C. for 2 h. LCMS showed the starting material was consumed completely and the MS of desired product was detected. The crude product was purified by prep-HPLC (column: Welch Xtimate C18 100*25 mm*3 um; mobile phase: [water(0.05% HCl)-ACN]; B %: 15%-40%,8 min). Compound 2-[[3-morpholinosulfonyl-6-(4-pyrazol-1-ylphenyl)-4-quinolyl]amino]benzoic acid (14.90 mg, 25.07 umol, 35.26% yield, 99.60% purity, HCl) was obtained as a yellow solid. ¹H NMR (400 MHz, DMSO-d₆) δ ppm=10.59 (br s, 1H), 9.10 (s, 1H), 8.57 (d, J=2.45 Hz, 1H), 8.25-8.31 (m, 1H), 8.17-8.22 (m, 1H), 8.07 (dd, J=7.89, 1.53 Hz, 1H), 7.89 (d, J=8.68 Hz, 2H), 7.82 (d, J=1.83 Hz, 1H), 7.77 (d, J=1.47 Hz, 1H), 7.38-7.48 (m, 3H), 7.18 (t, J=7.58 Hz, 1H), 6.87 (d, J=8.31 Hz, 1H), 6.55-6.58 (m, 1H), 3.51-3.58 (m, 2H), 3.40-3.47 (m, 2H), 3.01-3.19 (m, 4H). MS (M+H)⁺=556.1.

### Example 31—Synthesis of Compound 179A

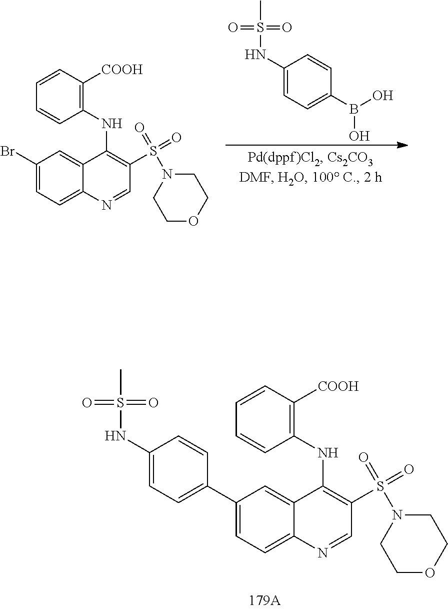 [SMILES: O=C(O)c1ccccc1Nc1c(S(=O)(=O)N2CCOCC2)cnc2ccc(Br)cc12 ; CS(=O)(=O)Nc1ccc(B(O)O)cc1 ; CS(=O)(=O)Nc1ccc(-c2ccc3ncc(S(=O)(=O)N4CCOCC4)c(Nc4ccccc4C(=O)O)c3c2)cc1]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added CS₂CO₃ (69.49 mg, 213.27 umol, 3 eq), Pd(dppf)Cl₂ (5.20 mg, 7.11 umol, 0.1 eq) and [4-(methanesulfonamido)phenyl]boronic acid (15.29 mg, 71.09 umol, 1 eq) was bubbled with N₂ for 1 minutes, the mixture was stirred at 100° C. for 2 h. LCMS showed the starting material was consumed completely and the MS of desired product was detected. The crude product was purified by prep-HPLC (column: Welch Xtimate C18 100*25 mm*3 um; mobile phase: [water(0.05% HCl)-ACN]; B %: 10%-40%,8 min) Compound 2-[[6-[4-(methanesulfonamido)phenyl]-3-morpholinosulfonyl-4-quinolyl]amino]benzoic acid (16.60 mg, 26.15 umol, 36.78% yield, 97.52% purity, HCl) was obtained as a yellow solid. ¹H NMR (400 MHz, DMSO-d₆) δ ppm=10.62 (br s, 1H), 9.95 (s, 1H), 9.10 (s, 1H), 8.16-8.26 (m, 2H), 8.08 (d, J=7.88 Hz, 1H), 7.75 (s, 1H), 7.44 (br t, J=7.75 Hz, 1H), 7.26-7.31 (m, 2H), 7.13-7.25 (m, 3H), 6.90 (br d, J=8.13 Hz, 1H), 3.51-3.59 (m, 2H), 3.40-3.51 (m, 2H), 3.06-3.20 (m, 4H), 3.02 (s, 3H). MS (M+H)⁺=583.0.

### Example 32—Synthesis of Compound 180A

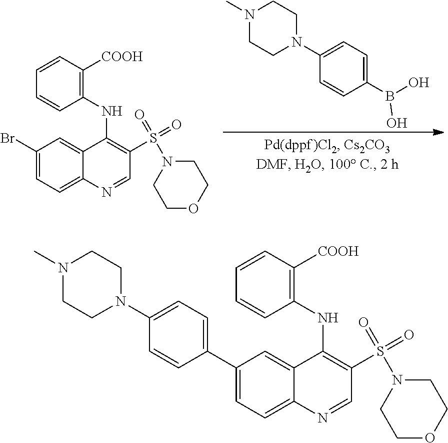 [SMILES: O=C(O)c1ccccc1Nc1c(S(=O)(=O)N2CCOCC2)cnc2ccc(Br)cc12 ; CN1CCN(c2ccc(B(O)O)cc2)CC1 ; CN1CCN(c2ccc(-c3ccc4ncc(S(=O)(=O)N5CCOCC5)c(Nc5ccccc5C(=O)O)c4c3)cc2)CC1]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and [4-(4-methylpiperazin-1-yl)phenyl]boronic acid (15.64 mg, 71.09 umol, 1 eq) was bubbled with N₂ for 1 minute, the mixture was stirred at 100° C. for 2 h. LCMS showed the starting material was consumed completely and the MS of desired product was detected. The crude product was purified by prep-HPLC (column: Welch Xtimate C18 100*25 mm*3 um; mobile phase: [water(0.05% HCl)-ACN]; B %: 5%-35%,8 min). Compound 2-[[6-[4-(4-methylpiperazin-1-yl)phenyl]-3-morpholinosulfonyl-4-quinolyl]amino]benzoic acid (10.90 mg, 16.85 umol, 23.70% yield, 96.47% purity, HCl) was obtained as a yellow solid. ¹H NMR (400 MHz, DMSO-d₆) δ ppm=10.52 (br s, 2H), 9.05 (s, 1H), 8.11-8.26 (m, 2H), 8.05 (br d, J=7.82 Hz, 1H), 7.71 (s, 1H), 7.39 (br t, J=7.70 Hz, 1H), 7.24 (br d, J=7.58 Hz. 2H), 7.14 (br t, J=7.58 Hz, 1H), 7.01 (br d, J=7.58 Hz, 2H), 6.82 (br d, J=7.58 Hz, 1H), 3.91 (br s, 2H), 3.76-3.81 (m, 2H), 3.43-3.52 (m, 4H), 3.09 (br d, J=8.44 Hz, 8H), 2.80 (br d, J=2.93 Hz, 3H). MS (M+H)⁺=588.3

### Example 33—Synthesis of Compound 181A

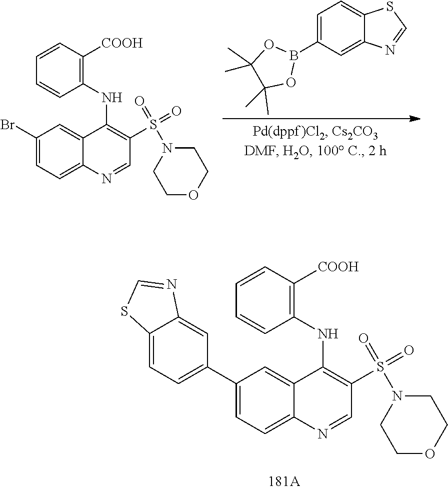 [SMILES: O=C(O)c1ccccc1Nc1c(S(=O)(=O)N2CCOCC2)cnc2ccc(Br)cc12 ; CC1(C)OB(c2ccc3scnc3c2)OC1(C)C ; O=C(O)c1ccccc1Nc1c(S(=O)(=O)N2CCOCC2)cnc2ccc(-c3ccc4scnc4c3)cc12]

To a stirred solution of 2-[(6-bromo-3-morpholinosulfonyl-4-quinolyl)amino]benzoic acid (40 mg, 81.24 umol, 1 eq) in DMF (0.5 mL) and H₂O (0.1 mL) was added 5-(4,4,5,5-tetramethyl-1,3,2-dioxaborolan-2-yl)-1,3-benzothiazole (21.22 mg, 81.24 umol, 1 eq), Cs₂CO₃ (79.41 mg, 243.73 umol, 3 eq), Pd(dppf)Cl₂ (5.94 mg, 8.12 umol, 0.1 eq), the mixture was bubbled with N₂ for 1 minute, and stirred at 100° C. for 2 h. LCMS showed the starting material was consumed completely and desired MS was detected. The reaction mixture was filtered, and filtrate was purified directly. The filtrate was purified by prep-HPLC (column: Welch Xtimate C18 100*25 mm*3 um; mobile phase: [water(0.05% HCl)-ACN]; B %: 15%-45%,8 min). Compound 2-[[6-(1,3-benzothiazol-5-yl)-3-morpholinosulfonyl-4-quinolyl]amino]benzoic acid (19.30 mg, 32.01 umol, 39.40% yield, 96.71% purity, HCl) was obtained as a yellow solid. ¹H NMR (400 MHz, DMSO-d₆) δ=9.38 (s, 1H), 9.08 (s, 1H), 8.32 (dd, J=2.0, 8.8 Hz, 1H), 8.18 (dd, J=8.6, 11.9 Hz, 2H), 8.05 (dd, J=1.5, 7.9 Hz, 1H), 7.93 (d, J=1.4 Hz, 1H), 7.84 (d, J=1.9 Hz, 1H), 7.48-7.39 (m, 2H), 7.22 (t, J=7.6 Hz, 1H), 6.90 (d, J=8.1 Hz, 1H), 3.57-3.49 (m, 2H), 3.45-3.38 (m, 2H), 3.10 (dt, J=3.3, 6.2 Hz, 4H). MS (M+H)⁺=547.0.

### Example 34—Synthesis of Compound 182A

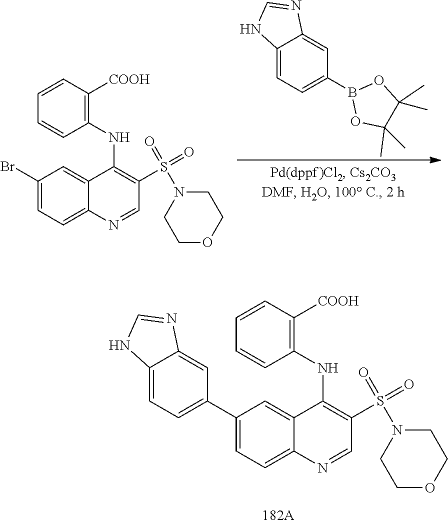 [SMILES: O=C(O)c1ccccc1Nc1c(S(=O)(=O)N2CCOCC2)cnc2ccc(Br)cc12 ; CC1(C)OB(c2ccc3[nH]cnc3c2)OC1(C)C ; O=C(O)c1ccccc1Nc1c(S(=O)(=O)N2CCOCC2)cnc2ccc(-c3ccc4[nH]cnc4c3)cc12]

To a solution of 2-[(6-bromo-3-morpholinosulfonyl-4-quinolyl)amino]benzoic acid (40 mg, 81.24 umol, 1 eq) in H₂O (0.1 mL) and DMF (0.5 mL) was added Cs₂CO₃ (79.41 mg, 243.73 umol, 3 eq), Pd(dppf)Cl₂ (5.94 mg, 8.12 umol, 0.1 eq) and 6-(4,4,5,5-tetramethyl-1,3,2-dioxaborolan-2-yl)-1H-benzimidazole (19.83 mg, 81.24 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min) Compound 2-[[6-(1H-benzimidazol-5-yl)-3-morpholinosulfonyl-4-quinolyl]amino]benzoic acid (3.20 mg, 5.58 umol, 6.87% yield, 98.70% purity, HCl) was obtained as a yellow solid. ¹H NMR (400 MHz, DMSO-d₆) δ ppm=9.43-9.49 (m, 1H), 9.09 (s, 1H), 8.25-8.29 (m, 1H), 8.20-8.24 (m, 1H), 8.04 (dd, J=7.94, 1.56 Hz, 1H), 7.84 (d, J=8.63 Hz, 1H), 7.80 (d, J=1.75 Hz, 1H), 7.76 (s, 1H), 7.47 (dd, J=8.69, 1.44 Hz, 1H), 7.37-7.43 (m, 1H), 7.19 (t, J=7.63 Hz, 1H), 6.85 (br d, J=8.13 Hz, 1H), 3.47-3.56 (m, 2H), 3.35-3.45 (m, 2H), 2.99-3.16 (m, 4H). MS (M+H)⁺=530.0

### Example 35—Synthesis of Compound 183A

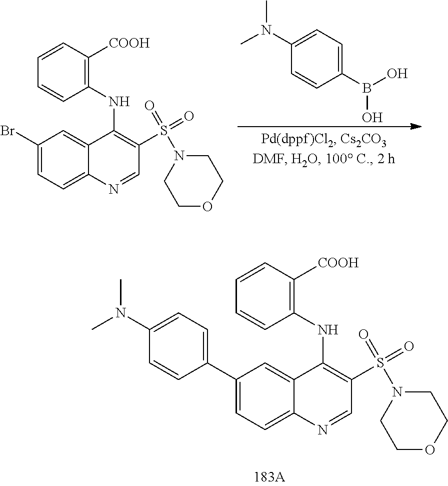 [SMILES: O=C(O)c1ccccc1Nc1c(S(=O)(=O)N2CCOCC2)cnc2ccc(Br)cc12 ; CN(C)c1ccc(B(O)O)cc1 ; CN(C)c1ccc(-c2ccc3ncc(S(=O)(=O)N4CCOCC4)c(Nc4ccccc4C(=O)O)c3c2)cc1]

To a solution of 2-[(6-bromo-3-morpholinosulfonyl-4-quinolyl)amino]benzoic acid (35 mg, 71.09 umol, 1 eq) in H₂O (0.1 mL) and DMF (0.5 mL) was added Cs₂CO₃ (69.49 mg, 213.27 umol, 3 eq), Pd(dppf)Cl₂ (5.20 mg, 7.11 umol, 0.1 eq) and [4-(dimethylamino)phenyl]boronic acid; hydrochloride (14.32 mg, 71.09 umol, 1 eq), was bubbled with N₂ for 1 minute, the mixture was stirred at 100° C. for 2 h. LCMS showed the starting material was consumed completely and desired product was detected. The reaction mixture was concentrate in vacuum. The crude product was purified by prep-HPLC (column: Welch Xtimate C18 100*25 mm*3 um; mobile phase: [water(0.05% HCl)-ACN]; B %: 10%-40%,8 min) The crude product was purified by prep-HPLC (column: Welch Xtimate C18 100*25 mm*3 um; mobile phase: [water(0.05% HCl)-ACN]; B %: 10%-40%,8 min) Compound 2-[[6-[4-(dimethylamino)phenyl]-3-morpholinosulfonyl-4-quinolyl]amino]benzoic acid (8.90 mg, 15.24 umol, 21.44% yield, 97.45% purity, HCl) was obtained as a yellow solid. ¹H NMR (400 MHz, DMSO-d₆) δ ppm=10.74 (br s, 1H), 9.09 (s, 1H), 8.18-8.30 (m, 2H), 8.10 (dd, J=7.88, 1.50 Hz, 1H), 7.66 (d, J=1.50 Hz, 1H), 7.45-7.51 (m, 1H), 7.31 (t, J=7.44 Hz, 1H), 7.18 (br d, J=8.50 Hz, 2H), 7.08 (br d, J=8.25 Hz, 1H), 6.90 (br s, 2H), 3.55-3.63 (m, 2H), 3.45-3.53 (m, 2H), 3.07-3.30 (m, 4H), 2.96 (s, 6H). MS (M+H)⁺=533.2.

### Example 36—Synthesis of Compound 184A

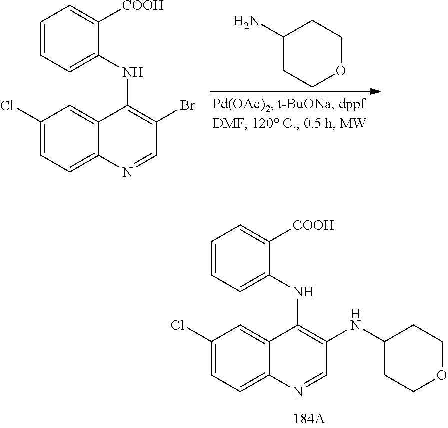 [SMILES: O=C(O)c1ccccc1Nc1c(Br)cnc2ccc(Cl)cc12 ; NC1CCOCC1 ; O=C(O)c1ccccc1Nc1c(NC2CCOCC2)cnc2ccc(Cl)cc12]

2-[(3-bromo-6-chloro-4-quinolyl)amino]benzoic acid (50 mg, 132.41 umol, 1 eq), tetrahydropyran-4-amine (20.09 mg, 198.61 umol, 1.5 eq), Pd(OAc)₂ (2.97 mg, 13.24 umol, 0.1 eq), DPPF (7.34 mg, 13.24 umol, 0.1 eq) and t-BuONa (38.17 mg, 397.23 umol, 3 eq) were taken up into a microwave tube in DMF (2 mL). The sealed tube was heated at 120° C. for 30 min under microwave. LCMS showed the starting material was consumed completely and desired MS was detected. The reaction mixture was filtered, the filtrate was purified by prep-HPLC (column: Welch Xtimate C18 100*25 mm*3 um; mobile phase: [water(0.05% HCl)-ACN]; B %: 15%-35%,8 min). Compound 2-[[6-chloro-3-(tetrahydropyran-4-ylamino)-4-quinolyl]amino]benzoic acid (4.58 mg, 10.33 umol, 7.80% yield, 98.33% purity, HCl) was obtained as a brown oil. ¹H NMR (400 MHz, DMSO-d₆) δ=9.59 (br s, 1H), 8.92 (s, 1H), 8.11 (br d, J=8.9 Hz, 1H), 7.97 (dd, J=1.1, 7.8 Hz, 1H), 7.65-7.57 (m, 2H), 7.38-7.25 (m, TH), 6.92 (t, J=7.6 Hz, TH), 6.36 (br d, J=8.3 Hz, 1H), 3.83 (br d, J=10.5 Hz, 3H), 3.38 (br s, 2H), 1.79 (br d, J=1.9 Hz, 2H), 1.53-1.37 (m, 2H). MS (M+H)⁺=398.1.

### Example 37—Synthesis of Compound 185A

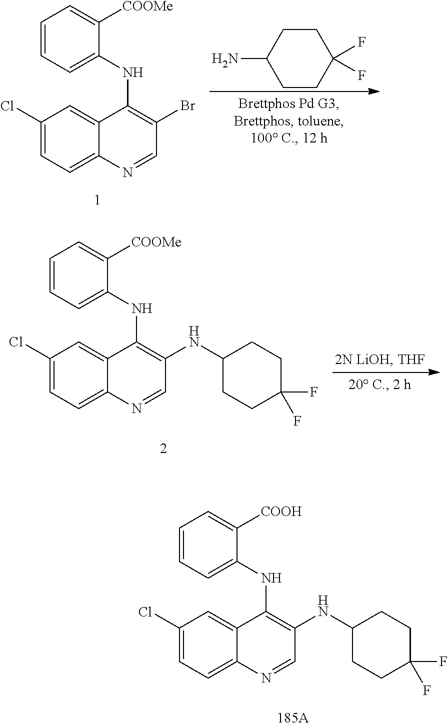 [SMILES: COC(=O)c1ccccc1Nc1c(Br)cnc2ccc(Cl)cc12 ; NC1CCC(F)(F)CC1 ; COC(=O)c1ccccc1Nc1c(NC2CCC(F)(F)CC2)cnc2ccc(Cl)cc12 ; O=C(O)c1ccccc1Nc1c(NC2CCC(F)(F)CC2)cnc2ccc(Cl)cc12]

Step 1. Synthesis of methyl 2-[[6-chloro-3-[(4,4-difluorocyclohexyl)amino]-4-quinolyl]amino]benzoate (2): To a solution of methyl 2-[(3-bromo-6-chloro-4-quinolyl)amino]benzoate (80 mg, 204.27 umol, 1 eq) in toluene (1 mL) was added BRETTPHOS (10.96 mg, 20.43 umol, 0.1 eq), BrettPhos Pd G3 (18.52 mg, 20.43 umol, 0.1 eq), sodium; 2-methylpropan-2-olate (39.26 mg, 408.53 umol, 2 eq) and 4,4-difluorocyclohexanamine (24.85 mg, 183.84 umol, 0.9 eq) was bubbled with N₂ for 1 minute, the mixture was stirred at 100° C. for 12 h. LCMS showed the starting material was consumed completely and the MS of desired product was detected. The reaction mixture was concentrated in vacuum. The crude product was purified by prep-HPLC (column: Welch Xtimate C18 100*25 mm*3 um; mobile phase:[water (0.05% HCl)-ACN]; B %: 40%-70%,8 min) Compound methyl 2-[[6-chloro-3-[(4,4-difluorocyclohexyl)amino]-4-quinolyl]amino]benzoate (17 mg, 33.10 umol, 16.20% yield, 93.91% purity, HCl) was obtained as a yellow solid. MS (M+H)=446.1.

Step 2. Synthesis of 2-[[6-chloro-3-[(4,4-difluorocyclohexyl)amino]-4-quinolyl]amino]benzoic acid (185A): To a solution of methyl 2-[[6-chloro-3-[(4,4-difluorocyclohexyl)amino]-4-quinolyl]amino]benzoate (15 mg, 33.64 umol, 1 eq) in THF (0.3 mL) was added LiOH (2 M, 33.64 uL, 2 eq), the mixture was stirred at 20° C. for 2 h. LCMS showed the starting material was consumed completely and the MS of desired product was detected. The reaction mixture was concentrated in vacuum. The crude product was purified by prep-HPLC (column: Welch Xtimate C18 100*25 mm*3 um; mobile phase: [water (0.05% HCl)-ACN]; B %: 30%-70%, 8 min) Compound 2-[[6-chloro-3-[(4,4-difluorocyclohexyl)amino]-4-quinolyl]amino]benzoic acid (2.87 mg, 6.13 umol, 18.22% yield, 100% purity, HCl) was obtained as a yellow solid. ¹H NMR (400 MHz, DMSO-d₆) δ ppm 9.42 (br s, 1H), 8.88 (s, 1H), 7.94-8.03 (m, 2H), 7.44-7.58 (m, 2H), 7.28 (t, J=7.58 Hz, 1H), 6.75-6.91 (m, 1H), 6.22 (br s, 1H), 5.62 (br s, 1H), 3.88 (br s, 1H), 1.83-2.04 (m, 6H), 1.56 (br d, J=9.05 Hz, 2H). MS (M+H)⁺=432.1

### Example 38—Synthesis of Compound 186A

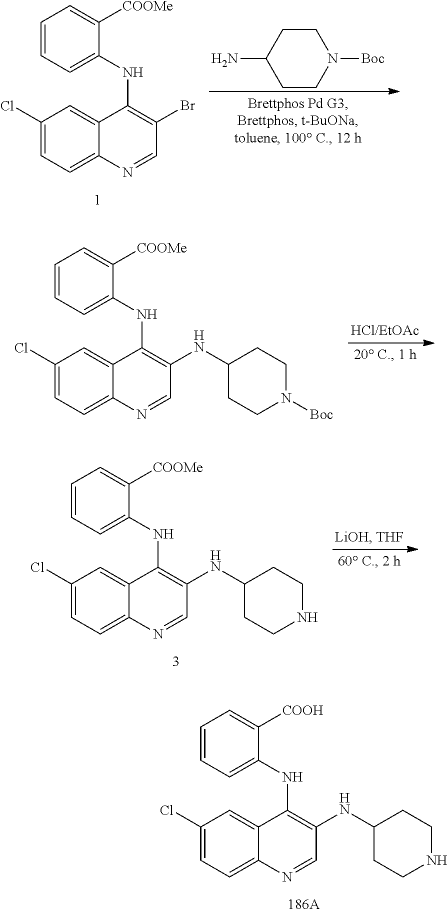 [SMILES: COC(=O)c1ccccc1Nc1c(Br)cnc2ccc(Cl)cc12 ; CC(C)(C)OC(=O)N1CCC(N)CC1 ; COC(=O)c1ccccc1Nc1c(NC2CCN(C(=O)OC(C)(C)C)CC2)cnc2ccc(Cl)cc12 ; COC(=O)c1ccccc1Nc1c(NC2CCNCC2)cnc2ccc(Cl)cc12 ; O=C(O)c1ccccc1Nc1c(NC2CCNCC2)cnc2ccc(Cl)cc12]

Step 1. Synthesis of tert-butyl 4-[[6-chloro-4-(2-methoxycarbonylanilino)-3-quinolyl]amino]piperidine-1-carboxylate (2): To a solution of methyl 2-[(3-bromo-6-chloro-4-quinolyl)amino]benzoate (200 mg, 510.67 umol, 1 eq) in toluene (3 mL) was added tert-butyl 4-aminopiperidine-1-carboxylate (102.27 mg, 510.67 umol, 1 eq), BRETTPHOS (27.41 mg, 51.07 umol, 0.1 eq), BrettPhos Pd G3 (46.29 mg, 51.07 umol, 0.1 eq), sodium; 2-methylpropan-2-olate (98.15 mg, 1.02 mmol, 2 eq), the mixture was bubbled with N₂ for 1 minute, the mixture was stirred at 100° C. for 12 h. LCMS showed starting material was consumed completely and the MS of desired product was detected. The reaction mixture was concentrated in vacuum. The crude product was purified by prep-HPLC (column: Welch Xtimate C18 100*25 mm*3 um; mobile phase: [water (0.04% HCl)-ACN]; B %: 40%-60%,8 min) Compound tert-butyl 4-[[6-chloro-4-(2-methoxycarbonylanilino)-3-quinolyl]amino]piperidine-1-carboxylate (25 mg, 48.92 umol, 9.58% yield) was obtained as a yellow solid. MS (M+H)⁺=511.3

Step 2. Synthesis of methyl 2-[[6-chloro-3-(4-piperidylamino)-4-quinolyl]amino]benzoate (3): A solution of tert-butyl 4-[[6-chloro-4-(2-methoxycarbonylanilino)-3-quinolyl]amino]piperidine-1-carboxylate (15 mg, 29.35 umol, 1 eq) in HCl/EtOAc (1.0 mL) was stirred at 20° C. for 1 h. LCMS showed the starting material was consumed completely and the MS of desired product was detected. The reaction mixture was concentrated in vacuum. Compound methyl 2-[[6-chloro-3-(4-piperidylamino)-4-quinolyl]amino]benzoate (10 mg, 24.34 umol, 82.91% yield) was obtained as a yellow solid. MS (M+H)⁺=411.4

Step 3. Synthesis of 2-[[6-chloro-3-(4-piperidylamino)-4-quinolyl]amino]benzoic acid (186A): To a solution of methyl 2-[[6-chloro-3-(4-piperidylamino)-4-quinolyl]amino]benzoate (10 mg, 24.34 umol, 1 eq) in THF (0.5 mL) was added LiOH (582.83 ug, 24.34 umol, 1 eq), the mixture was stirred at 60° C. for 2 h. LCMS showed starting material was consumed completely and the MS of desired product was detected. The reaction mixture was concentrated in vacuum. The crude product was purified by prep-HPLC (column: Welch Xtimate C18 100*25 mm*3 um; mobile phase: [water (0.04% HCl)-ACN]; B %: 5%-40%,8 min) Compound 2-[[6-chloro-3-(4-piperidylamino)-4-quinolyl]amino]benzoic acid (2.05 mg, 16.15 umol, 66.38% yield, 100% purity, HCl) was obtained as a yellow solid. ¹H NMR (400 MHz, DMSO-d₆+D₂O) δ ppm 8.87 (s, 1H), 7.91-8.02 (m, 2H), 7.49-7.56 (m, 2H), 7.25-7.34 (m, 1H), 6.86 (t, J=7.50 Hz, 1H), 6.23 (d, J=8.50 Hz, 1H), 3.88-3.95 (m, 1H), 3.29 (br d, J=12.38 Hz, 2H), 2.94-3.01 (m, 2H), 2.03 (br d, J=12.76 Hz, 2H), 1.56-1.66 (m, 2H). MS (M+H)⁺=397.2.

### Example 39—Synthesis of Compound 188A

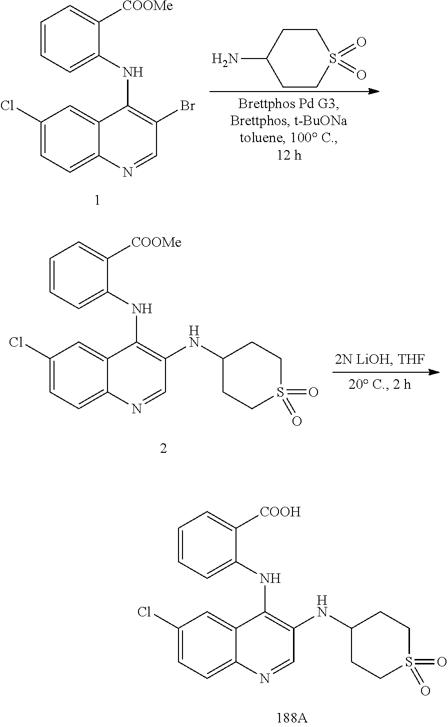 [SMILES: COC(=O)c1ccccc1Nc1c(Br)cnc2ccc(Cl)cc12 ; NC1CCS(=O)(=O)CC1 ; COC(=O)c1ccccc1Nc1c(NC2CCS(=O)(=O)CC2)cnc2ccc(Cl)cc12 ; O=C(O)c1ccccc1Nc1c(NC2CCS(=O)(=O)CC2)cnc2ccc(Cl)cc12]

Step 1. Synthesis of methyl 2-[[6-chloro-3-[(1,1-dioxothian-4-yl)amino]-4-quinolyl]amino]benzoate (2): To a solution of 1,1-dioxothian-4-amine (76.20 mg, 510.67 umol, 1 eq) in toluene (0.5 mL) was added methyl 2-[(3-bromo-6-chloro-4-quinolyl)amino]benzoate (200.00 mg, 510.67 umol, 1 eq), BRETTPHOS (27.41 mg, 51.07 umol, 0.1 eq), BrettPhos Pd G3 (46.29 mg, 51.07 umol, 0.1 eq), sodium; 2-methylpropan-2-olate (98.15 mg, 1.02 mmol, 2 eq) was bubbled with N₂ for 1 minute, the mixture was stirred at 100° C. for 12 h. LCMS showed starting material was consumed completely and the MS of desired product was detected. The reaction mixture was concentrated in vacuum. The crude product was purified by prep-HPLC (column: Welch Xtimate C18 100*25 mm*3 um; mobile phase: [water (0.04% HCl)-ACN]; B %: 10%-40%, 8 min) Compound methyl 2-[[6-chloro-3-[(1,1-dioxothian-4-yl)amino]-4-quinolyl]amino]benzoate (25 mg, 50.36 umol, 9.86% yield, HCl) was obtained as a yellow solid. MS (M+H)⁺=460.2.

Step 2. Synthesis of 2-[[6-chloro-3-[(1,1-dioxothian-4-yl)amino]-4-quinolyl]amino]benzoic acid (188A): To a solution of methyl 2-[[6-chloro-3-[(1,1-dioxothian-4-yl)amino]-4-quinolyl]amino]benzoate (10 mg, 21.74 umol, 1 eq) in THF (0.5 mL) was added LiOH (2 M, 10.87 uL, 1 eq) was stirred at 60° C. for 2 h. LCMS showed the starting material was consumed completely and the MS of desired product was detected. The reaction mixture was concentrated in vacuum. The crude product was purified by prep-HPLC (column: Welch Xtimate C18 100*25 mm*3 um; mobile phase:[water (0.04% HCl)-ACN]; B %: 5%-35%,8 min) Compound 2-[[6-chloro-3-[(1,1-dioxothian-4-yl)amino]-4-quinolyl]amino]benzoic acid (1.43 mg, 2.96 umol, 13.63% yield, 100% purity, HCl) was obtained as a yellow solid. ¹H NMR (400 MHz, DMSO-d₆) δ ppm 8.84 (s, 1H), 7.90-8.06 (m, 2H), 7.53-7.64 (m, 2H), 7.28-7.36 (m, 1H), 6.91 (t, J=7.50 Hz, 1H), 6.84-6.99 (m, 1H), 6.30 (d, J=8.38 Hz, 1H), 3.18-3.32 (m, 2H), 3.08 (br d, J=12.51 Hz, 2H), 2.09-2.19 (m, 2H), 2.02 (br s, 2H). MS (M+H)⁺=446.1

### Example 40—Synthesis of Compound 191A

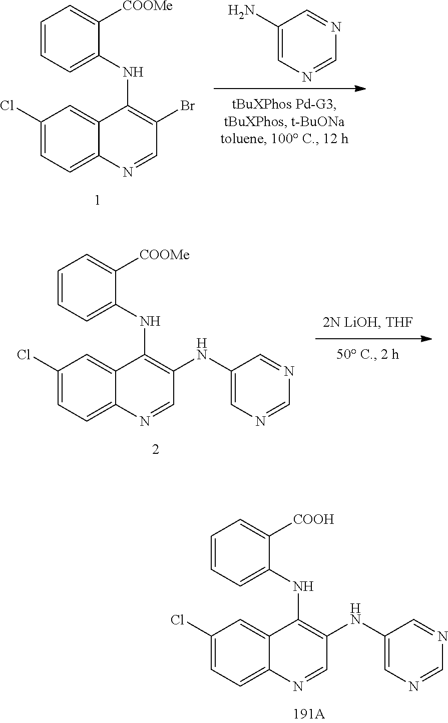 [SMILES: COC(=O)c1ccccc1Nc1c(Br)cnc2ccc(Cl)cc12 ; Nc1cncnc1 ; COC(=O)c1ccccc1Nc1c(Nc2cncnc2)cnc2ccc(Cl)cc12 ; O=C(O)c1ccccc1Nc1c(Nc2cncnc2)cnc2ccc(Cl)cc12]

Step 1. Synthesis of methyl 2-[[6-chloro-3-(pyrimidin-5-ylamino)-4-quinolyl]amino]benzoate (2): To a solution of methyl 2-[(3-bromo-6-chloro-4-quinolyl)amino]benzoate (100 mg, 255.33 umol, 1 eq) 2-methylbutan-2-ol (1.5 ML) was added sodium; 2-methylpropan-2-olate (49.08 mg, 510.67 umol, 2 eq), tBuXPhos Pd G3 (20.28 mg, 25.53 umol, 0.1 eq) and t-Bu Xphos (10.84 mg, 25.53 umol, 0.1 eq) and pyrimidin-5-amine (24.28 mg, 255.33 umol, 1 eq) was bubbled with N₂ for 1 minute, the mixture was stirred at 100° C. for 12 h. LCMS showed starting material was consumed completely and the MS of desired product was detected. The reaction mixture was concentrated in vacuum. The crude product was purified by prep-HPLC (column: Welch Xtimate C18 100*25 mm*3 um; mobile phase: [water (0.04% HCl)-ACN]; B %: 10%-30%, 8 min) Compound methyl 2-[[6-chloro-3-(pyrimidin-5-ylamino)-4-quinolyl]amino]benzoate (15 mg, 33.91 umol, 13.28% yield, HCl) was obtained as a yellow solid. MS (M+H)⁺=406.2

Step 2. Synthesis of 2-[[6-chloro-3-(pyrimidin-5-ylamino)-4-quinolyl]amino]benzoic acid (191A): To a solution of methyl 2-[[6-chloro-3-(pyrimidin-5-ylamino)-4-quinolyl]amino]benzoate (5 mg, 12.32 umol, 1 eq) in THF (0.3 mL) was added LiOH (2 M, 12.32 uL, 2 eq), the mixture was stirred at 50° C. for 2 h. LCMS showed the starting material was consumed completely and the MS of desired product was detected. The reaction mixture was concentrated in vacuum. The crude product was purified by prep-HPLC (column: Welch Xtimate C18 100*25 mm*3 um; mobile phase: [water(0.04% HCl)-ACN]; B %: 10%-40%,8 min). Compound 2-[[6-chloro-3-(pyrimidin-5-ylamino)-4-quinolyl]amino]benzoic acid (0.82 mg, 1.75 umol, 14.19% yield, 91.32% purity, HCl) was obtained as a yellow solid. ¹H NMR (400 MHz, DMSO-d₆) δ ppm 10.27 (s, 1H), 8.82 (s, 1H), 8.61 (s, 1H), 8.45 (s, 1H), 8.22 (s, 1H), 8.08 (d, J=8.92 Hz, 1H), 7.92 (s, 2H), 7.88 (br d, J=9.17 Hz, 1H), 7.72 (d, J=7.58 Hz, 1H), 7.34 (t, J=7.52 Hz, 1H), 6.98 (t, J=7.58 Hz, 1H), 6.66 (d, J=8.31 Hz, 1H). MS (M+H)⁺=392.1.

### Example 41—Synthesis of 204A

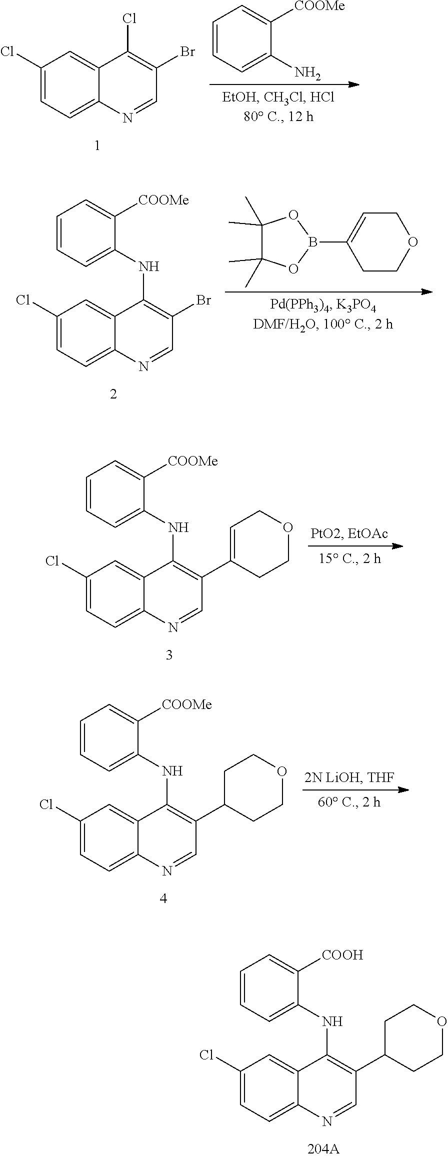

Step 1. Synthesis of methyl 2-[(3-bromo-6-chloro-4-quinolyl)amino]benzoate (2): A solution of 3-bromo-4,6-dichloro-quinoline (350 mg, 1.26 mmol, 1 eq), methyl 2-aminobenzoate (191.04 mg, 1.26 mmol, 163.28 uL, 1 eq), HCl (12 M, 10.53 uL, 0.1 eq) in EtOH (5 mL) and CHCl₃ (1 mL) was stirred at 80° C. for 12 h. LCMS showed the starting material was consumed completely and desired MS was detected. The reaction mixture was concentrate in vacuum. Compound methyl 2-[(3-bromo-6-chloro-4-quinolyl)amino]benzoate (450 mg, 1.15 mmol, 90.92% yield) was obtained as a yellow solid. MS (M+H)⁺=393.2.

Step 2. Synthesis of methyl 2-[[6-chloro-3-(3,6-dihydro-2H-pyran-4-yl)-4-quinolyl]amino]benzoate (3): To a stirred solution of methyl 2-[(3-bromo-6-chloro-4-quinolyl)amino]benzoate (250 mg, 638.33 umol, 1 eq) in DMF (2 mL) and H₂O (0.4 mL) was added 2-(3,6-dihydro-2H-pyran-4-yl)-4,4,5,5-tetramethyl-1,3,2-dioxaborolane (134.10 mg, 638.33 umol, 1 eq), Pd(PPh₃)₄ (73.76 mg, 63.83 umol, 0.1 eq), K₃PO₄ (406.49 mg, 1.91 mmol, 3 eq), the mixture was bubbled with N₂ for 1 minute, and stirred at 100° C. for 2 h. LCMS showed the starting material was consumed completely and desired MS was detected. The reaction mixture was poured into water (50 mL). The aqueous phase was extracted with ethyl acetate (50 mL*2). The combined organic phase was dried with anhydrous Na₂SO₄, filtered and concentrated in vacuum. The residue was purified by flash column (ISCO 10 g silica, 25-30% Ethyl acetate in Petroleum ether, gradient over 15 min). Based on TLC (Petroleum ether Ethyl acetate=1/1, R_{f}=0.37). Compound methyl 2-[[6-chloro-3-(3,6-dihydro-2H-pyran-4-yl)-4-quinolyl]amino]benzoate (160 mg, 405.22 umol. 63.48% yield) was obtained as a yellow solid. MS (M+H)⁺=395.2.

Step 3. Synthesis of methyl 2-[(6-chloro-3-tetrahydropyran-4-yl-4-quinolyl)amino]benzoate (4): A solution of methyl 2-[[6-chloro-3-(3,6-dihydro-2H-pyran-4-yl)-4-quinolyl]amino]benzoate (60 mg, 151.96 umol, 1 eq), PtO2 (30 mg, 132.11 umol, 8.69e-1 eq) in EtOAc (1 mL) was stirred at 20° C. under N₂, the mixture was bubbled with H₂ for 3 times, and stirred at 20° C. for 2 h under H₂ (15 psi). LCMS showed the starting material was consumed completely and desired MS was detected. The reaction mixture was filtered, and filtrate was concentrate in vacuum. Compound methyl 2-[(6-chloro-3-tetrahydropyran-4-yl-4-quinolyl)amino]benzoate (40 mg, 100.79 umol, 66.33% yield) was obtained as a yellow oil. MS (M+H)⁺=395.2.

Step 4. Synthesis of 2-[(6-chloro-3-tetrahydropyran-4-yl-4-quinolyl)amino]benzoic acid (204A): To a solution of methyl 2-[(6-chloro-3-tetrahydropyran-4-yl-4-quinolyl)amino]benzoate (30.00 mg, 75.59 umol, 1 eq) in THF (2 mL) was added LiOH·H₂O (6.34 mg, 151.18 umol, 2 eq), the mixture was stirred at 50° C. for 2 h. LCMS showed starting material was consumed completely and the MS of desired product was detected. The reaction mixture was concentrated in vacuum. The crude product was purified by prep-HPLC (column: Phenomenex Gemini-NX 80*30 mm*3 um; mobile phase: [water(0.04% HCl)-ACN]; B %: 15%-45%,8 min). Compound 2-[(6-chloro-3-tetrahydropyran-4-yl-4-quinolyl)amino]benzoic acid (2.40 mg, 35.17 umol, 46.52% yield, 98.30% purity, HCl) was obtained as a yellow solid. ¹H NMR (400 MHz, DMSO-d₆+D₂O) δ=8.90 (s, 1H), 8.02 (d, J=9.0 Hz, 1H), 7.92 (dd, J=1.2, 7.8 Hz, 1H), 7.71 (dd, J=2.3, 8.9 Hz, 1H), 7.66 (d, J=2.2 Hz, 1H), 7.19-7.11 (m, 1H), 6.77 (t, J=7.5 Hz, 1H), 6.06 (d, J=8.3 Hz, 1H), 3.96-3.85 (m, 2H), 3.43-3.31 (m, 1H), 3.28-3.17 (m, 1H), 3.16-3.04 (m, 1H), 2.04-1.88 (m, 1H), 1.85-1.71 (m, 1H), 1.70-1.63 (m, 1H), 1.59-1.49 (m, 1H). MS (M+H)⁺=383.2.

### Example 42—Synthesis of Compound 204A-BP

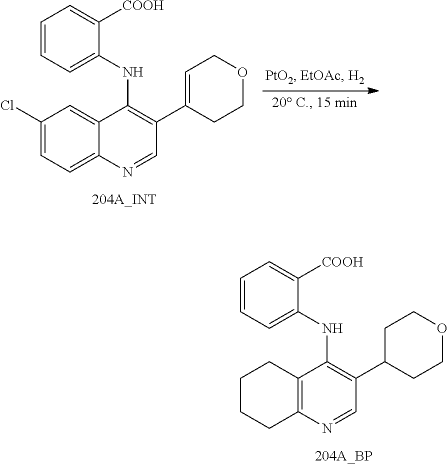 [SMILES: O=C(O)c1ccccc1Nc1c(C2=CCOCC2)cnc2ccc(Cl)cc12 ; O=C(O)c1ccccc1Nc1c(C2CCOCC2)cnc2c1CCCC2]

A solution of 2-[[6-chloro-3-(3,6-dihydro-2H-pyran-4-yl)-4-quinolyl]amino]benzoic acid (80 mg, 210.07 umol, 1 eq) and PtO2 (47.70 mg, 210.07 umol, 1 eq) in EtOAc (1 mL) was stirred at 20° C. under N₂, and purged with H₂ for 3 times, and stirred at 20° C. for 15 min under H₂ (15 psi). LCMS showed the product was detected. The reaction mixture was filtered, and filtrate was concentrate in vacuum. The residue was purified by prep-HPLC (column: Phenomenex luna C18 80*40 mm*3 um; mobile phase: [water(0.04% HCl)-ACN]; B %: 30%-37%,5.5 min). Afford 10 mg crude product. The crude product was purified by prep-HPLC (column: Phenomenex Luna C18 100*30 mm*3 um; mobile phase: [water(0.04% HCl)-ACN]; B %: 10%-30%,8 min). Compound 2-[(3-tetrahydropyran-4-yl-5,6,7,8-tetrahydroquinolin-4-yl)amino]benzoic acid (3.03 mg, 7.57 umol, 3.60% yield, 97.19% purity, HCl) was obtained as a yellow solid. ¹H NMR (400 MHz, DMSO-d6) δ=9.75 (s, 1H), 8.39 (s, 1H), 7.98-7.90 (m, 1H), 7.55-7.43 (m, 1H), 7.11 (t, J=7.6 Hz, 1H), 6.71 (d, J=8.2 Hz, 1H), 3.92 (br d, J=10.9 Hz, 2H), 3.34-3.22 (m, 2H), 3.08-2.90 (m, 3H), 2.36-2.25 (m, 2H), 1.97-1.44 (m, 8H). MS (M+H)⁺=353.2.

### Example 43—Synthesis of Compound 204A-INT

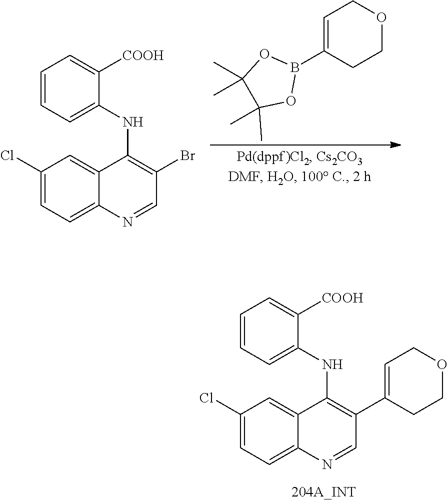 [SMILES: O=C(O)c1ccccc1Nc1c(Br)cnc2ccc(Cl)cc12 ; CC1(C)OB(C2=CCOCC2)OC1(C)C ; O=C(O)c1ccccc1Nc1c(C2=CCOCC2)cnc2ccc(Cl)cc12]

To a solution of 2-[(3-bromo-6-chloro-4-quinolyl)amino]benzoic acid (100 mg, 264.82 umol, 1 eq) in DMF (2.5 mL) and H₂O (0.5 mL) was added Cs₂CO₃ (258.85 mg, 794.45 umol, 3 eq), Pd(dppf)Cl₂ (19.38 mg, 26.48 umol, 0.1 eq) and 2-(3,6-dihydro-2H-pyran-4-yl)-4,4,5,5-tetramethyl-1,3,2-dioxaborolane (50.07 mg, 238.34 umol, 0.9 eq) was bubbled with N₂ for 1 minutes, the mixture was stirred at 100° C. for 2 h. LCMS showed the starting material was consumed completely and the Ms of desired product was detected. The reaction mixture was concentrated in vacuum. The crude product was purified by prep-HPLC (column: Phenomenex luna C18 80*40 mm*3 um; mobile phase: [water(0.04% HCl)-ACN]; B %: 35%-55%,7 min) Afford crude product (28 mg) The crude product was purified by prep-HPLC (column: Phenomenex luna C18 80*40 mm*3 um; mobile phase: [water(0.04% HCl)-ACN]; B %: 15%-45%,7 min) Compound 2-[[6-chloro-3-(3,6-dihydro-2H-pyran-4-yl)-4-quinolyl]amino]benzoic acid (20 mg, 46.93 umol, 17.72% yield, 97.91% purity, HCl) was obtained as a yellow solid. 1H NMR (400 MHz, DMSO-d₆) δ ppm=8.71 (br s, 1H), 8.59-8.65 (m, 1H), 8.06-8.13 (m, 1H), 8.00-8.05 (m, 1H), 7.97 (d, J=8.00 Hz, 1H), 7.51-7.61 (m, 1H), 7.28-7.38 (m, 1H), 7.35 (t, J=7.57 Hz, 1H), 7.12 (br d, J=7.75 Hz, 1H), 5.77 (br s, 1H), 3.82 (br s, 2H), 3.02-3.18 (m, 2H), 2.03 (br s, 2H). MS (M+H)⁺=381.1

### Example 44—Synthesis of Compound 205A

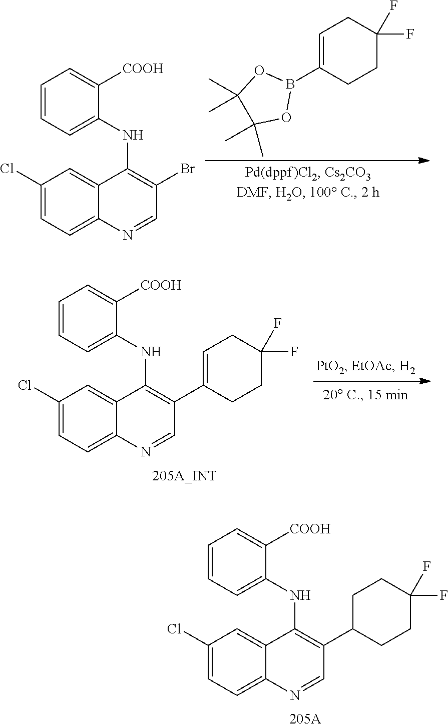 [SMILES: O=C(O)c1ccccc1Nc1c(Br)cnc2ccc(Cl)cc12 ; CC1(C)OB(C2=CCC(F)(F)CC2)OC1(C)C ; O=C(O)c1ccccc1Nc1c(C2=CCC(F)(F)CC2)cnc2ccc(Cl)cc12 ; O=C(O)c1ccccc1Nc1c(C2CCC(F)(F)CC2)cnc2ccc(Cl)cc12]

Step 1: Synthesis of 2-[[6-chloro-3-(4,4-difluorocyclohexen-1-yl)-4-quinolyl]amino]benzoic acid (205A INT): To a solution of 2-[(3-bromo-6-chloro-4-quinolyl)amino]benzoic acid (100 mg, 264.82 umol, 1 eq) in DMF (2.5 mL) and H₂O (0.5 mL) was added Cs₂CO₃ (258.85 mg, 794.45 umol, 3 eq), Pd(dppf)Cl₂ (19.38 mg, 26.48 umol, 0.1 eq) and 2-(4,4-difluorocyclohexen-1-yl)-4,4,5,5-tetramethyl-1,3,2-dioxaborolane (58.17 mg, 238.34 umol, 0.9 eq) was bubbled with N₂ for 1 minute, the mixture was stirred at 100° C. for 2 h. LCMS showed the starting material was consumed completely and the Ms of desired product was detected. The reaction mixture was concentrated in vacuum. The crude product was purified by prep-HPLC (column: Phenomenex luna C18 80*40 mm*3 um; mobile phase: [water(0.04% HCl)-ACN]; B %: 25%-55%,7 min) Compound 2-[[6-chloro-3-(4,4-difluorocyclohexen-1-yl)-4-quinolyl]amino]benzoic acid (29.5 mg, 63.45 umol, 23.96% yield, 97.07% purity, HCl) was obtained as a yellow solid. ¹H NMR (400 MHz, DMSO-d6) δ ppm=10.35 (br s, 1H), 8.60-8.74 (m, 2H), 8.10-8.19 (m, 1H), 7.92-8.05 (m, 2H), 7.55 (t, J=7.63 Hz, 1H), 7.31 (br t, J=7.44 Hz, 1H), 7.10 (br d, J=6.50 Hz, 1H), 5.64 (br s, 1H), 2.20-2.41 (m, 4H), 1.46 (br s, 2H). MS (M+H)⁺=415.1.

Step 2: Synthesis of 2-[[6-chloro-3-(4,4-difluorocyclohexyl)-4-quinolyl]amino]benzoic acid (205A): A solution of 2-[[6-chloro-3-(4,4-difluorocyclohexan-1-yl)-4-quinolyl]amino]benzoic acid (80 mg, 192.85 umol, 1 eq) and PtO2 (20 mg, 88.08 umol, 4.57e-1 eq) in EtOAc (1 mL) was purged with H₂ for 3 times, and stirred at 20° C. for 15 min under H₂ (15 psi). LCMS showed the starting material was consumed completely and desired MS was detected. The reaction mixture was filtered, and filtrate was concentrate in vacuum. The residue was purified by prep-HPLC (column: Welch Xtimate C18 100*25 mm*3 um; mobile phase: [water (0.04% HCl)-ACN]; B %: 10%-40%,8 min). Compound 2-[[6-chloro-3-(4,4-difluorocyclohexyl)-4-quinolyl]amino]benzoic acid (0.23 mg, 5.07e-1 umol, 2.63e-1% yield, 100% purity, HCl) was obtained as a yellow solid. ¹H NMR (400 MHz, DMSO-d₆+D₂O) δ=8.88 (s, 1H), 8.05 (d, J=8.6 Hz, 1H), 7.99-7.92 (m, 1H), 7.82-7.74 (m, 1H), 7.69 (s, 1H), 7.36-7.28 (m. 1H), 7.01-6.92 (m, 1H), 6.37 (br d, J=8.4 Hz, 1H), 3.02-2.93 (m, 1H), 2.16-2.03 (m, 2H), 1.99-1.58 (m, 6H). MS (M+H)⁺=417.1.

### Example 45—Synthesis of Compound 206A-INT

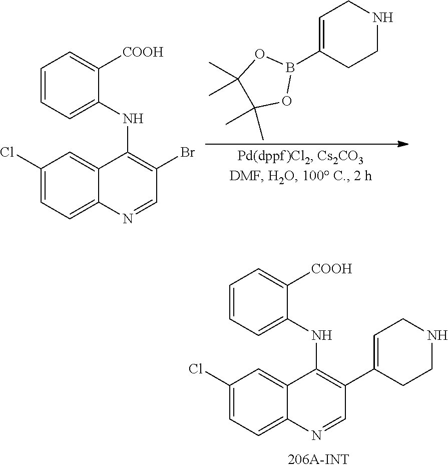 [SMILES: O=C(O)c1ccccc1Nc1c(Br)cnc2ccc(Cl)cc12 ; CC1(C)OB(C2=CCNCC2)OC1(C)C ; O=C(O)c1ccccc1Nc1c(C2=CCNCC2)cnc2ccc(Cl)cc12]

To a solution of 2-[(3-bromo-6-chloro-4-quinolyl)amino]benzoic acid (100 mg, 264.82 umol, 1 eq) in H₂O (0.5 mL) and DMF (2.5 mL) was added Cs₂CO₃ (258.85 mg, 794.45 umol, 3 eq), Pd(dppf)Cl₂ (19.38 mg, 26.48 umol, 0.1 eq) and 4-(4,4,5,5-tetramethyl-1,3,2-dioxaborolan-2-yl)-1,2,3,6-tetrahydropyridine (49.83 mg, 238.34 umol, 0.9 eq), was bubbled with N₂ for 1 minutes, the mixture was stirred at 100° C. for 2 h. LCMS showed the starting material was consumed completely and the Ms of desired product was detected. The reaction mixture was concentrated in vacuum. The crude product was purified by prep-HPLC (column: Phenomenex luna C18 80*40 mm*3 um; mobile phase: [water(0.04% HCl)-ACN]; B %: 12%-42%,7 min) Compound 2-[[6-chloro-3-(1,2,3,6-tetrahydropyridin-4-yl)-4-quinolyl]amino]benzoic acid (25 mg, 58.98 umol, 22.27% yield, 98.22% purity, HCl) was obtained as a yellow solid. ¹H NMR (400 MHz, DMSO-d₆) δ ppm 10.29 (br s, 1H), 9.14 (br s, 2H), 8.56 (s, 1H), 8.49 (br s, 1H), 8.17 (d, J=9.01 Hz, 1H), 7.89-8.05 (m, 2H), 7.53 (br t, J=7.25 Hz, 1H), 7.28 (br s, 1H), 7.03 (br s, 1H), 5.83 (br s, 1H), 3.31-3.35 (m, 4H), 2.33 (br d, J=1.88 Hz, 2H). MS (M+H⁺=380.0

### Example 46—Synthesis of Compound 206A

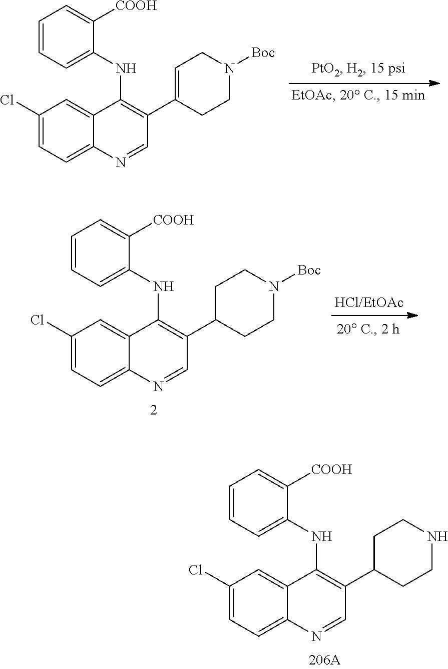 [SMILES: CC(C)(C)OC(=O)N1CC=C(c2cnc3ccc(Cl)cc3c2Nc2ccccc2C(=O)O)CC1 ; CC(C)(C)OC(=O)N1CCC(c2cnc3ccc(Cl)cc3c2Nc2ccccc2C(=O)O)CC1 ; O=C(O)c1ccccc1Nc1c(C2CCNCC2)cnc2ccc(Cl)cc12]

Step 1. Synthesis of 2-[[3-(1-tert-butoxycarbonyl-4-piperidyl)-6-chloro-4-quinolyl]amino]benzoic acid (2): A solution of 2-[[3-(1-tert-butoxycarbonyl-3,6-dihydro-2H-pyridin-4-yl)-6-chloro-4-quinolyl]amino]benzoic acid (80 mg, 166.68 umol, 1 eq), PtO2 (37.85 mg, 166.68 umol, 1 eq) in EtOAc (2 mL), the mixture was bubbled with H₂ for 3 times, and stirred at 15° C. for 15 min under H₂ (15 psi). LCMS showed the starting material was remained and 20% desired MS was detected. The reaction mixture was filtered, and filtrate was purified by prep-HPLC (column: Phenomenex luna C18 80*40 mm*3 um; mobile phase: [water(0.04% HCl)-ACN]; B %: 32%-50%,7 min). Compound 2-[[3-(1-tert-butoxycarbonyl-4-piperidyl)-6-chloro-4-quinolyl]amino]benzoic acid (10 mg, 19.29 umol, 11.57% yield, HCl) was obtained as a yellow solid. MS (M+H)⁺=482.3.

Step 2. Synthesis of 2-[[6-chloro-3-(4-piperidyl)-4-quinolyl]amino]benzoic acid (206A): A solution of 2-[[3-(1-tert-butoxycarbonyl-4-piperidyl)-6-chloro-4-quinolyl]amino]benzoic acid (10 mg, 20.75 umol, 1 eq) in HCl/EtOAc (4 M, 2 mL, 385.58 eq) was stirred at 20° C. for 1 h. LCMS showed the starting material was consumed completely and desired MS was detected. The reaction mixture was concentrate in vacuum. The residue was purified by prep-HPLC (column: Phenomenex luna C18 80*40 mm*3 um; mobile phase: [water(0.04% HCl)-ACN]; B %: 5%-45%,7 min). Compound 2-[[6-chloro-3-(4-piperidyl)-4-quinolyl]amino]benzoic acid (0.83 mg, 1.98 umol, 9.56% yield, 100% purity, HCl) was obtained as a yellow oil. ¹H NMR (400 MHz, DMSO-d₆+D₂O) δ=8.83 (s, 1H), 8.07 (d, J=9.0 Hz, 1H), 7.98 (dd, J=1.6, 7.9 Hz, 1H), 7.83 (dd, J=2.3, 9.0 Hz, 1H), 7.66 (d, J=2.3 Hz, 1H), 7.38 (dt, J=1.6, 7.8 Hz, 1H), 7.05 (t, J=7.6 Hz, 1H), 6.52 (d, J=8.4 Hz, 1H), 3.36 (br d, J=12.6 Hz, 2H), 3.21-3.11 (m, 1H), 2.99-2.78 (m, 2H), 2.17-1.81 (m, 4H). MS (M+H)⁺=382.1.

### Example 47—Synthesis of Compound 207A

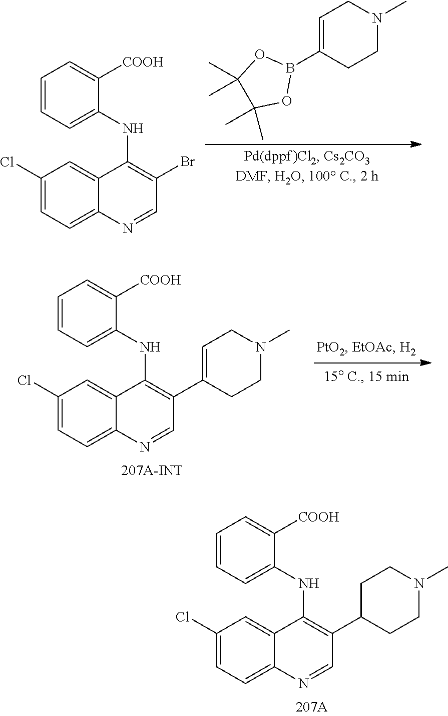

Step 1. Synthesis of 2-[[6-chloro-3-(1-methyl-3,6-dihydro-2H-pyridin-4-yl)-4-quinolyl]amino]benzoic acid (207A INT): To a solution of 2-[(3-bromo-6-chloro-4-quinolyl)amino]benzoic acid (200 mg, 529.63 umol, 1 eq) in DMF (2.5 mL) and H₂O (0.5 mL) was added Cs₂CO₃ (517.70 mg, 1.59 mmol, 3 eq), Pd(dppf)Cl₂ (38.75 mg, 52.96 umol, 0.1 eq) and 1-methyl-4-(4,4,5,5-tetramethyl-1,3,2-dioxaborolan-2-yl)-3,6-dihydro-2H-pyridine (118.17 mg, 529.63 umol, 1 eq) was bubbled with N₂ for 1 minute, the mixture was stirred at 100° C. for 2 h. LCMS showed the starting material was consumed completely and the Ms of desired product was detected. The reaction mixture was concentrated in vacuum. The crude product was purified by prep-HPLC (column: Welch Xtimate C18 100*25 mm*3 um; mobile phase: [water(0.05% HCl)-ACN]; B %: 1%-15%,8 min). Afford crude product (34 mg) The crude product was purified by prep-HPLC (column: Phenomenex luna C18 80*40 mm*3 um; mobile phase: [water(0.04% HCl)-ACN]; B %: 13%-33%,7 min). Compound 2-[[6-chloro-3-(1-methyl-3,6-dihydro-2H-pyridin-4-yl)-4-quinolyl]amino]benzoic acid (25 mg, 57.41 umol, 10.84% yield, 98.82% purity, HCl) was obtained as a yellow solid. ¹H NMR (400 MHz, DMSO-d₆) δ ppm=11.05 (br s, 1H), 10.37 (br s, 1H), 8.59 (s, 1H), 8.52 (br s, 1H), 8.21 (d, J=9.13 Hz, 1H), 8.02 (dd, J=9.01, 1.88 Hz, 1H), 7.96 (dd, J=7.82, 1.31 Hz, 1H), 7.56 (br t, J=7.50 Hz, 1H), 7.33 (br t, J=7.32 Hz, 1H), 7.12 (br s, 1H), 5.80 (br s, 1H), 3.66 (br s, 2H), 3.20-3.28 (m, 1H), 3.02 (br s, 1H), 2.66 (br d, J=3.25 Hz, 3H), 2.28-2.43 (m, 2H). MS (M+H)⁺=394.0.

Step 2. Synthesis of 2-[[6-chloro-3-(1-methyl-4-piperidyl)-4-quinolyl]amino]benzoic acid (207A): A solution 2-[[6-chloro-3-(1-methyl-3,6-dihydro-2H-pyridin-4-yl)-4-quinolyl]amino]benzoic acid (10 mg, 25.39 umol, 1 eq) and PtO2 (2 mg, 8.81 umol, 3.47e-1 eq) in EtOAc (1 mL) was stirred at 15° C. under N₂, and the mixture was purged with H₂ for 3 times, and stirred at 15° C. for 15 min under H₂ (15 psi). LCMS showed the starting material was consumed completely and desired MS was detected. The reaction mixture was filtered, and filtrate was purified by directly. The filtrate was purified by prep-HPLC (column: Phenomenex Gemini NX-C18(75*30 mm*3 um); mobile phase:[water(0.04% HCl)-ACN]; B %: 3%-30%,8 min). Compound 2-[[6-chloro-3-(1-methyl-4-piperidyl)-4-quinolyl]amino]benzoic acid (1.54 mg, 3.56 umol, 14.03% yield, 100% purity, HCl) was obtained as a yellow solid. ¹H NMR (400 MHz, DMSO-d₆) δ=10.38-10.27 (m, 1H), 9.95 (br s, 1H), 8.87 (s, 1H), 8.15 (d, J=8.9 Hz, 1H), 7.99 (d, J=7.9 Hz, 1H), 7.86 (dd, J=2.3, 8.9 Hz, 1H), 7.73 (d, J=2.2 Hz, 1H), 7.37 (br t, J=7.4 Hz, 1H), 7.05-6.98 (m, 1H), 6.58-6.44 (m, 1H), 3.51-3.47 (m, 2H), 3.18-3.13 (m, 1H), 3.02-2.93 (m, 2H), 2.74 (br d, J=4.5 Hz, 3H), 2.14-1.90 (m, 4H). MS (M+H)⁺=396.1.

### Example 48—Synthesis of Compound 208A_INT

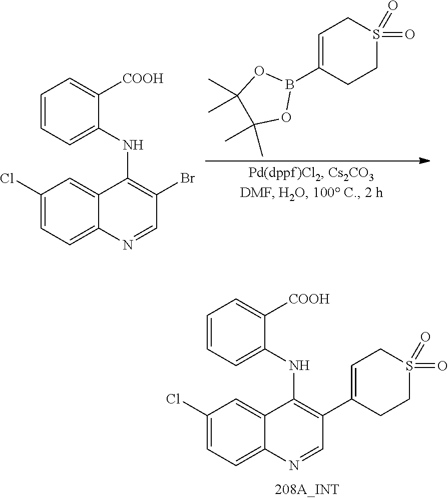 [SMILES: O=C(O)c1ccccc1Nc1c(Br)cnc2ccc(Cl)cc12 ; CC1(C)OB(C2=CCS(=O)(=O)CC2)OC1(C)C ; O=C(O)c1ccccc1Nc1c(C2=CCS(=O)(=O)CC2)cnc2ccc(Cl)cc12]

To a solution of 2-[(3-bromo-6-chloro-4-quinolyl)amino]benzoic acid (100 mg, 264.82 umol, 1 eq) in DMF (0.5 mL) and H₂O (0.1 mL) was added Cs₂CO₃ (258.85 mg, 794.45 umol, 3 eq), Pd(dppf)Cl₂ (19.38 mg, 26.48 umol, 0.1 eq) and 4-(4,4,5,5-tetramethyl-1,3,2-dioxaborolan-2-yl)-3,6-dihydro-2H-thiopyran 1,1-dioxide (68.36 mg, 264.82 umol, 1 eq) was bubbled with N₂ for 1 minute, the mixture was stirred at 100° C. for 2 h. LCMS showed the starting material was consumed completely and the Ms of desired product was detected. The reaction mixture was concentrated in vacuum. The crude product was purified by prep-HLC (column: Phenomenex luna C18 80*40 mm*3 um; mobile phase: [water(0.04% HCl)-ACN]; B %: 12%-42%,7 min)Afford crude product (25 mg) The crude product was purified by prep-HPLC (column: Phenomenex luna C18 80*40 mm*3 um; mobile phase: [water(0.04% HCl)-ACN]; B %: 18%-38%,7 min). Compound 2-[[6-chloro-3-(1,1-dioxo-3,6-dihydro-2H-thiopyran-4-yl)-4-quinolyl]amino]benzoic acid (10 mg, 20.77 umol, 7.84% yield, 96.65% purity, HCl) was obtained as a yellow solid. ¹H NMR (400 MHz, DMSO-d₆) δ ppm=10.45 (br s, 1H), 8.71 (br s, 1H), 8.63 (s, 1H), 8.17 (d, J=9.01 Hz, 1H), 8.02-8.07 (m, 1H), 7.99 (d, J=7.75 Hz, 1H), 7.61 (br t, J=7.63 Hz, 1H), 7.39 (br t, J=7.50 Hz, 1H), 7.19 (br d, J=7.75 Hz, 1H), 5.73 (br s, 1H), 3.56 (br s, 4H), 2.67 (br s, 2H). MS (M+H)⁺=429.0

### Example 49—Synthesis of 208A

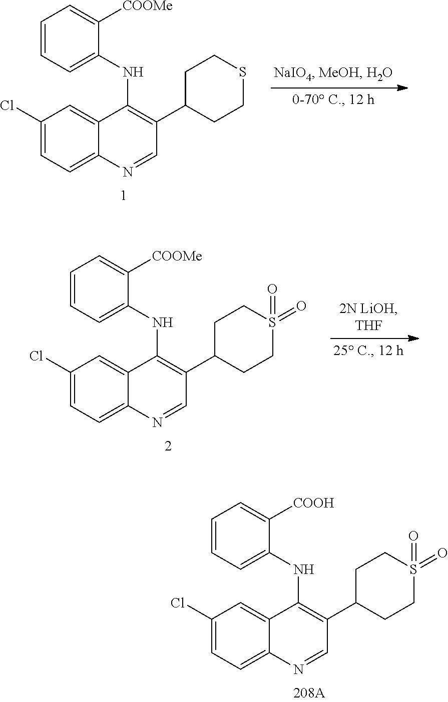 [SMILES: COC(=O)c1ccccc1Nc1c(C2CCSCC2)cnc2ccc(Cl)cc12 ; COC(=O)c1ccccc1Nc1c(C2CCS(=O)(=O)CC2)cnc2ccc(Cl)cc12 ; O=C(O)c1ccccc1Nc1c(C2CCS(=O)(=O)CC2)cnc2ccc(Cl)cc12]

Step 1. Synthesis of methyl 2-[[6-chloro-3-(1,1-dioxothian-4-yl)-4-quinolyl]amino]benzoate (2): To a stirred solution of methyl 2-[(6-chloro-3-tetrahydrothiopyran-4-yl-4-quinolyl)amino]benzoate (15 mg, 36.33 umol, 1 eq) in MeOH (0.2 mL) and H₂O (0.2 mL) was added NaIO₄ (31.08 mg, 145.30 umol, 8.05 uL, 4 eq) at 0° C., then the mixture was stirred at 70° C. for 12 h. LCMS showed the starting material was consumed completely and desired Ms was detected. The reaction mixture was poured into water (5 mL). The aqueous phase was extracted with ethyl acetate (5 mL*2). The combined organic phase was dried with anhydrous Na₂SO₄, filtered and concentrated in vacuum. Compound methyl 2-[[6-chloro-3-(1,1-dioxothian-4-yl)-4-quinolyl]amino]benzoate (15 mg, 33.71 umol, 92.81% yield) was obtained as a yellow solid. MS (M+H)⁺=445.2.

Step 2. Synthesis of 2-[[6-chloro-3-(1,1-dioxothian-4-yl)-4-quinolyl]amino]benzoic acid (208A): To a stirred solution of methyl 2-[[6-chloro-3-(1,1-dioxothian-4-yl)-4-quinolyl]amino]benzoate (15 mg, 33.71 umol, 1 eq) in THF (0.5 mL) and MeOH (0.5 mL) was added LiOH·H₂O (2 M, 33.71 uL, 2 eq) at 25° C., then the mixture was stirred at 60° C. for 1 h. LCMS showed the starting material was consumed completely and desired Ms was detected. The reaction mixture was adjusted pH-4 by adding 2N HCl. Then the mixture was concentrate in vacuum. The residue was purified by prep-HPLC (column: Phenomenex luna C18 80*40 mm*3 um; mobile phase: [water(0.04% HCl)-ACN]; B %: 16%-41%,7 min). Compound 2-[[6-chloro-3-(1,1-dioxothian-4-yl)-4-quinolyl]amino]benzoic acid (8.50 mg, 17.88 umol, 53.04% yield, 98.32% purity, HCl) was obtained as a yellow solid. ¹H NMR (400 MHz, DMSO-d₆+D₂O) δ=8.80 (s, 1H), 8.05-7.95 (m, 2H), 7.85 (dd, J=2.3, 9.0 Hz, 1H), 7.63 (d, J=2.3 Hz, 1H), 7.50-7.43 (m, 1H), 7.26-7.17 (m, 1H), 6.81 (d, J=7.8 Hz, 1H), 3.26-3.06 (m, 5H), 2.54 (s, 1H), 2.16 (br d, J=1.6 Hz, 3H). MS (M+H)⁺=431.0.

### Example 50—Synthesis of Compound 209A

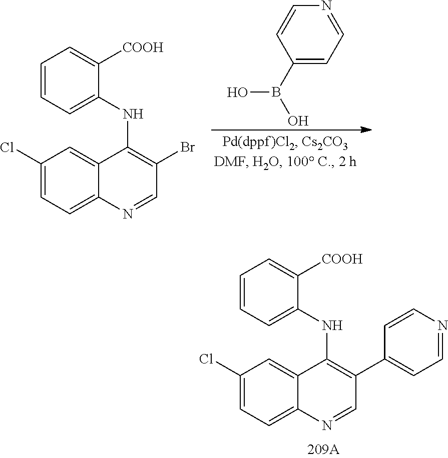 [SMILES: O=C(O)c1ccccc1Nc1c(Br)cnc2ccc(Cl)cc12 ; OB(O)c1ccncc1 ; O=C(O)c1ccccc1Nc1c(-c2ccncc2)cnc2ccc(Cl)cc12]

Synthesis of 2-[[6-chloro-3-(4-pyridyl)-4-quinolyl]amino]benzoic acid (209A): To a solution of 4-pyridylboronic acid (22.19 mg, 180.54 umol, 1 eq) in H₂O (0.2 mL) and DMF (1 mL) was added Cs₂CO₃ (176.47 mg, 541.62 umol, 3 eq), Pd(dppf)Cl₂ (13.21 mg, 18.05 umol, 0.1 eq) and 2-[(3-bromo-6-chloro-4-quinolyl)amino]benzoic acid (68.18 mg, 180.54 umol, 1 eq) was bubbled with N₂ for 1 minutes, the mixture was stirred at 100° C. for 2 h. LCMS showed the starting material was consumed completely and the Ms of desired product was detected. The reaction mixture was concentrated in vacuum. The crude product was purified by prep-HPLC (column: Phenomenex luna C18 80*40 mm*3 um; mobile phase: [water(0.04% HCl)-ACN]; B %: 15%-35%,7 min) Afford crude product (20 mg) The crude product was purified by prep-HPLC (column: Waters Xbridge BEH C18 100*30 mm*10 um; mobile phase: [water(0.04% HCl)-ACN]; B %: 4%-34%,8 min) Compound 2-[[6-chloro-3-(4-pyridyl)-4-quinolyl]amino]benzoic acid (4.32 mg, 11.11 umol, 6.15% yield, 96.64% purity) was obtained as a yellow solid. ¹H NMR (400 MHz, DMSO-d₆) δ ppm=10.10 (br s, 1H), 8.92 (s, 1H), 8.51 (d, J=5.99 Hz, 2H), 8.14 (d, J=8.92 Hz, 1H), 8.07 (d, J=2.32 Hz, 1H), 7.86 (dd, J=9.05, 2.32 Hz, 1H), 7.82 (dd, J=7.89, 1.53 Hz, 1H), 7.45-7.51 (m, 2H), 7.01-7.11 (m, 1H), 6.71 (t, J=7.52 Hz, 1H), 6.30 (d, J=8.31 Hz, 1H). MS (M+H)⁺=376.1

### Example 51—Synthesis of Compound 211A

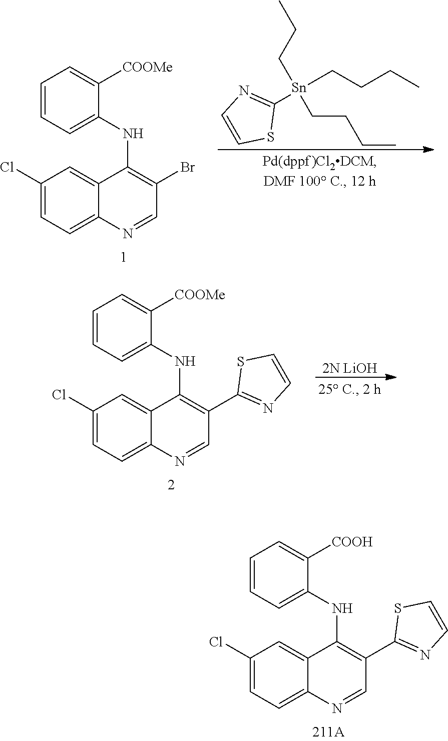 [SMILES: COC(=O)c1ccccc1Nc1c(Br)cnc2ccc(Cl)cc12 ; CCC[CH2][Sn]([CH2]CC)([CH2]CCC)[c]1nccs1 ; COC(=O)c1ccccc1Nc1c(-c2nccs2)cnc2ccc(Cl)cc12 ; "2N LiOH" ; O=C(O)c1ccccc1Nc1c(-c2nccs2)cnc2ccc(Cl)cc12]

Step 1: Synthesis of methyl 2-[(6-chloro-3-thiazol-2-yl-4-quinolyl)amino]benzoate (2): To a stirred solution of methyl 2-[(3-bromo-6-chloro-4-quinolyl)amino]benzoate (100 mg, 255.33 umol, 1 eq) in dioxane (5 mL) was added tributyl(thiazol-2-yl)stannane (95.54 mg, 255.33 umol, 1 eq), [2-(2-aminophenyl)phenyl]-chloropalladium; bis(1-adamantyl)-butyl-phosphane (17.07 mg, 25.53 umol, 0.1 eq) the mixture was bubbled with N₂ for 1 minute, and stirred at 110° C. for 12 h. LCMS showed the starting material was consumed completely and desired MS was detected. The reaction mixture was concentrate in vacuum. The residue was purified by prep-HPLC (column: Phenomenex Gemini-NX 80*40 mm*3 um; mobile phase: [water(10 mM NH₄HCO₃)-ACN]; B %: 40%-70%,8 min). Compound methyl 2-[(6-chloro-3-thiazol-2-yl-4-quinolyl)amino]benzoate (25 mg, 57.83 umol, 22.65% yield, HCl) was obtained as a yellow solid. MS (M+H)⁺=396.1.

### Synthesis of 2-[(6-chloro-3-thiazol-2-yl-4-quinolyl)amino]benzoic acid (211A)

A solution of methyl 2-[(6-chloro-3-thiazol-2-yl-4-quinolyl)amino]benzoate (20 mg, 50.52 umol, 1 eq), LiOH (2 M, 50.52 uL, 2 eq) in THF (0.5 mL) was stirred at 25° C. for 2 h. LCMS showed the starting material was consumed completely and desired MS was detected. The reaction mixture was concentrate in vacuum. The residue was purified by prep-HPLC (column: Welch Xtimate C18 100*25 mm*3 um; mobile phase: [water(0.05% HCl)-ACN]; B %: 15%-45%,8 min). Compound 2-[(6-chloro-3-thiazol-2-yl-4-quinolyl)amino]benzoic acid (9.15 mg, 20.87 umol, 41.31% yield, 95.41% purity, HCl) was obtained as a yellow solid. ¹H NMR (400 MHz, DMSO-d₆) δ=11.79 (br s, 1H), 9.48 (s, 1H), 8.26-8.18 (m, 1H), 8.03-7.97 (m, 2H), 7.97-7.91 (m, 2H), 7.78 (s, 1H), 7.35 (br t, J=7.8 Hz, 1H), 7.17 (br t, J=7.3 Hz, 1H), 6.81 (br d, J=8.1 Hz, 1H). MS (M+H)⁺=382.0.

### Example 52—Synthesis of Compound 212A

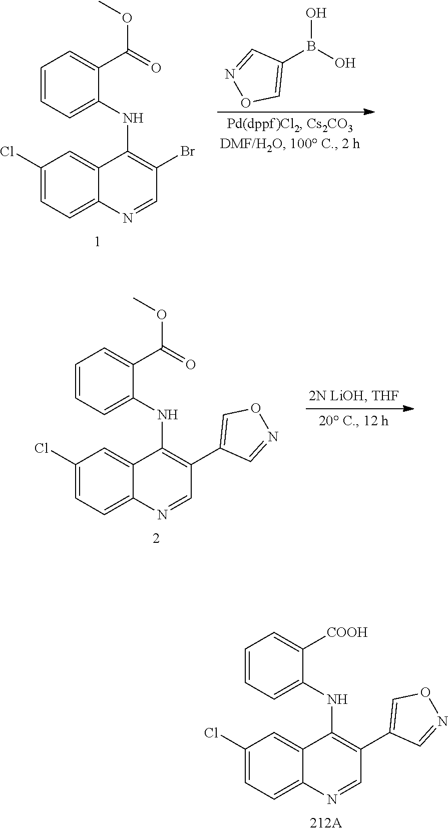 [SMILES: COC(=O)c1ccccc1Nc1c(Br)cnc2ccc(Cl)cc12 ; OB(O)c1cnoc1 ; COC(=O)c1ccccc1Nc1c(-c2cnoc2)cnc2ccc(Cl)cc12 ; O=C(O)c1ccccc1Nc1c(-c2cnoc2)cnc2ccc(Cl)cc12]

Step 1. Synthesis of methyl 2-[(6-chloro-3-isoxazol-4-yl-4-quinolyl)amino]benzoate (2): To a stirred solution of methyl 2-[(3-bromo-6-chloro-4-quinolyl)amino]benzoate (100 mg, 255.33 umol, 1 eq) in DMF (4 mL) and H₂O (1 mL) was added isoxazol-4-ylboronic acid (34.59 mg, 306.40 umol, 1.2 eq), Pd(dppf)Cl₂ (18.68 mg, 25.53 umol, 0.1 eq), Cs₂CO₃ (249.58 mg, 766.00 umol, 3 eq) the mixture was bubbled with N₂ for 1 minute, and stirred at 100° C. for 2 h. LCMS showed the starting material was consumed completely and desired MS was detected. The reaction mixture was filtered, and filtrate was purified directly. The residue was purified by prep-HPLC (column: Phenomenex luna C18 80*40 mm*3 um; mobile phase: [water(0.04% HCl)-ACN]; B %: 20%-40%,7 min). Compound methyl 2-[(6-chloro-3-isoxazol-4-yl-4-quinolyl)amino]benzoate (20 mg, 48.05 umol, 18.82% yield, HCl) was obtained as a yellow solid. MS (M+H)⁺=380.2.

Step 2. Synthesis of 2-[(6-chloro-3-isoxazol-4-yl-4-quinolyl)amino]benzoic acid (212A): A solution of methyl 2-[(6-chloro-3-isoxazol-4-yl-4-quinolyl)amino]benzoate (20 mg, 52.66 umol, 1 eq), LiOH (2 M, 52.66 uL, 2 eq) in THF (0.5 mL) was stirred at 20° C. for 12 h. LCMS showed the starting material was consumed completely and 10% desired MS was detected. The reaction mixture was concentrate in vacuum. The residue was purified by prep-HPLC (column: Phenomenex Gemini-NX C18 75*30 mm*3 um; mobile phase: [water(10 mMNH4HCO3)-ACN]; B %: 10%-40%,8 min). Compound 2-[(6-chloro-3-isoxazol-4-yl-4-quinolyl)amino]benzoic acid (240.00 ug, 6.14e-1 umol, 1.17% yield, 93.60% purity) was obtained as a yellow solid. ¹H NMR (400 MHz, DMSO-d₆+D₂O) δ=9.67 (d, J=2.8 Hz, 1H), 8.13 (d, J=2.8 Hz, 1H), 8.09 (s, 1H), 8.08-8.03 (m, 2H), 7.82-7.76 (m, 2H), 7.60-7.55 (m, 2H), 6.88-6.84 (m, 1H. MS (M+H)⁺=366.0.

### Example 53—Synthesis of Compound 216A

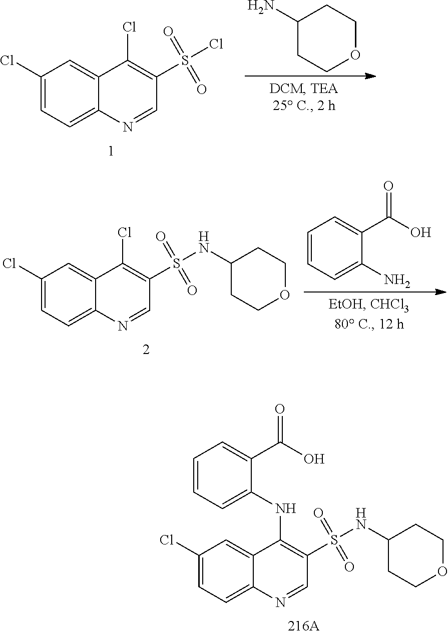

Step 1. Synthesis of 4,6-dichloro-N-tetrahydropyran-4-yl-quinoline-3-sulfonamide (2): to a solution of 4,6-dichloroquinoline-3-sulfonyl chloride (50 mg, 168.60 umol, 1 eq) in DCM (0.5 mL) was added tetrahydropyran-4-amine (17.05 mg, 168.60 umol, 1 eq) and TEA (51.18 mg, 505.80 umol, 70.40 uL, 3 eq), the mixture was stirred at 25° C. for 1 h. LCMS showed the starting material was consumed completely and the MS of desired product was detected. The reaction mixture was concentrate in vacuum. Compound 4,6-dichloro-N-tetrahydropyran-4-yl-quinoline-3-sulfonamide (50 mg, 138.41 umol, 82.09% yield) was obtained as a white solid. MS (M+H)+=361.2

Step 2. Synthesis of 2-[[6-chloro-3-(tetrahydropyran-4-ylsulfamoyl)-4-quinolyl]amino]benzoic acid (216A): To a solution of 4,6-dichloro-N-tetrahydropyran-4-yl-quinoline-3-sulfonamide (25 mg, 69.21 umol, 1 eq) in ACN (0.5 mL) was added 2-aminobenzoic acid (9.49 mg, 69.21 umol, 1 eq), the mixture was stirred at 80° C. for 12 h. LCMS showed the starting material was consumed completely and the MS of desired product was detected. The reaction mixture was concentrate in vacuum. The crude product was purified by prep-HPLC (column: Phenomenex luna C18 80*40 mm*3 um; mobile phase: [water (0.04% HCl)-ACN]; B %: 22%-48%,7 min). Compound 2-[[6-chloro-3-(tetrahydropyran-4-ylsulfamoyl)-4-quinolyl]amino]benzoic acid (25.2 mg, 49.79 umol, 71.95% yield, 98.47% purity, HCl) was obtained as a yellow solid. 1H NMR (400 MHz, DMSO-d6+D2O) δ ppm 9.13 (s, 1H), 8.09 (d, J=9.05 Hz, 1H), 8.03 (dd, J=7.95, 1.47 Hz, 1H), 7.87 (dd, J=9.11, 2.26 Hz, 1H), 7.51 (d, J=2.20 Hz, 1H), 7.31-7.38 (m, 1H), 7.12 (t, J=7.52 Hz, 1H), 6.61 (d, J=8.31 Hz, 1H), 3.61 (br t, J=12.47 Hz, 2H), 3.17-3.29 (m, 1H), 2.87-3.03 (m, 2H), 1.18-1.53 (m, 4H). MS (M+H)+=462.0

### Example 54—Synthesis of Compound 217A

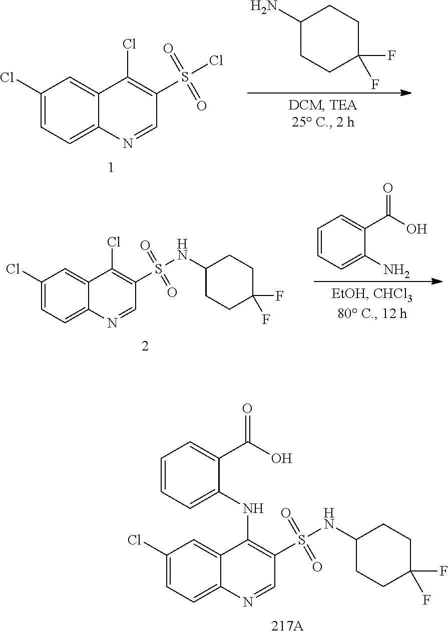 [SMILES: O=S(=O)(Cl)c1cnc2ccc(Cl)cc2c1Cl ; NC1CCC(F)(F)CC1 ; O=S(=O)(NC1CCC(F)(F)CC1)c1cnc2ccc(Cl)cc2c1Cl ; Nc1ccccc1C(=O)O ; O=C(O)c1ccccc1Nc1c(S(=O)(=O)NC2CCC(F)(F)CC2)cnc2ccc(Cl)cc12]

Step 1. Synthesis of 4,6-dichloro-N-(4,4-difluorocyclohexyl)quinoline-3-sulfonamide (2): To a solution of 4,6-dichloroquinoline-3-sulfonyl chloride (50 mg, 168.60 umol, 1 eq) in DCM (0.5 mL) was added 4,4-difluorocyclohexanamine; hydrochloride (28.93 mg, 168.60 umol, 1 eq) and TEA (51.18 mg, 505.80 umol, 70.40 uL, 3 eq), the mixture was stirred at 25° C. for 1 h. LCMS showed the starting material was consumed completely and the MS of desired product was detected. The reaction mixture was concentrate in vacuum. Compound 4,6-dichloro-N-(4,4-difluorocyclohexyl)quinoline-3-sulfonamide (45 mg, 113.85 umol, 67.53% yield) was obtained as a white solid. MS (M+H)⁺=395.2

Step 2. Synthesis of 2-[[6-chloro-3-[(4,4-difluorocyclohexyl)sulfamoyl]-4-quinolyl]amino]benzoic acid (217A): To a solution of 4,6-dichloro-N-(4,4-difluorocyclohexyl)quinoline-3-sulfonamide (30 mg, 75.90 umol, 1 eq) in ACN (0.5 mL) was added 2-aminobenzoic acid (10.41 mg, 75.90 umol, 1 eq), the mixture was stirred at 80° C. for 12 h. LCMS showed the starting material was consumed completely and the MS of desired product was detected. The reaction mixture was concentrate in vacuum. The crude product was purified by prep-HPLC (column: Welch Xtimate C18 100*25 mm*3 um; mobile phase: [water (0.05% HCl)-ACN]; B %: 20%-45%,8 min). Compound 2-[[6-[6-imino-4-(trifluoromethyl)-1H-pyridin-3-yl]-3-morpholinosulfonyl-4-quinolyl]amino]benzoic acid (2.80 mg, 4.59 umol, 5.65% yield, 100% purity, HCl) was obtained as a yellow solid. ¹H NMR (400 MHz, DMSO-d₆+D₂O) δ ppm 9.11 (s, 1H), 8.08 (d, J=9.05 Hz, 1H), 8.02 (dd, J=7.89, 1.53 Hz, 1H), 7.86 (dd, J=8.99, 2.26 Hz, 1H), 7.49 (d, J=2.20 Hz, 1H), 7.29-7.37 (m, 1H), 7.12 (t, J=7.58 Hz, 1H), 6.60 (d, J=8.19 Hz, 1H), 3.10-3.33 (m, 1H), 1.79 (br s, 2H), 1.36-1.69 (m, 5H), 1.15-1.34 (m, 1H). MS (M+H)⁺=496.0.

### Example 55—Synthesis of Compound 219A

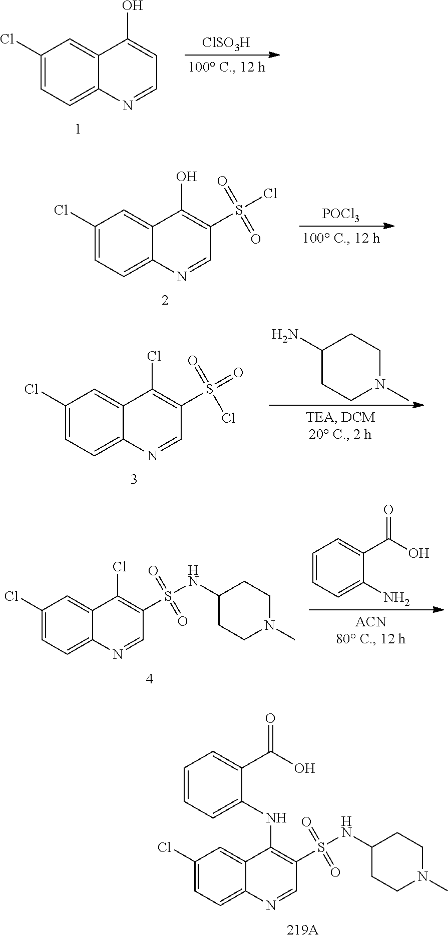

Step 1. Synthesis of 6-chloro-4-hydroxy-quinoline-3-sulfonyl chloride (2): A solution of 6-chloroquinolin-4-ol (5.3 g, 29.51 mmol, 1 eq) in HSO₃Cl (40 mL) was stirred at 100° C. for 12 h. LCMS showed the starting material was consumed completely and the MS of desired product was detected. The mixture was poured onto ice water (100 mL). Filtered, and filter cake was concentrate in vacuum Based on TLC (Petroleum ether:Ethyl acetate=10:1, R_{f}=0.49). Compound 6-chloro-4-hydroxy-quinoline-3-sulfonyl chloride (8.0 g, 28.77 mmol, 97.48% yield) was obtained as a yellow solid. MS (M+H)⁺=278.1.

Step 2. Synthesis of 4,6-dichloroquinoline-3-sulfonyl chloride (3): A solution of 6-chloro-4-hydroxy-quinoline-3-sulfonyl chloride (8 g, 28.77 mmol, 1 eq) in POCl₃ (50 mL) was stirred at 100° C. for 12 h. TLC (Petroleum ether:Ethyl acetate=3:1, R_{f}=0.50) showed starting material was consumed completely and new spot was formed. The reaction mixture was concentrated in vacuum. The residue was dissolved with ethyl acetate (100 mL). The mixture was poured into water (100 mL). The aqueous phase was extracted with ethyl acetate (200 mL*2). The combined organic phase was dried with Na₂SO₄, filtered and concentrated in vacuum. The residue was purified by flash column (ISCO 40 g silica, 10-35% Ethyl acetate in Petroleum ether, gradient over 30 min). Compound 4,6-dichloroquinoline-3-sulfonyl chloride (2.2 g, 7.42 mmol, 25.79% yield) was obtained as a white solid.

Step 3. 1,7-dichloro-N-(1-methyl-4-piperidyl)naphthalene-2-sulfonamide (4): To a solution of 4,6-dichloroquinoline-3-sulfonyl chloride (70 mg, 236.04 umol, 1 eq) in CH₂Cl₂ (2 mL) was added 1-methylpiperidin-4-amine (26.95 mg, 236.04 umol, 1 eq) and TEA (71.66 mg, 708.13 umol, 98.56 uL, 3 eq) was stirred at 25° C. for 2 h. LCMS showed the starting material was consumed completely and the MS of desired product was detected. The reaction mixture was concentrated in vacuum. Compound 1,7-dichloro-N-(1-methyl-4-piperidyl)naphthalene-2-sulfonamide (40 mg, 107.15 umol, 45.40% yield) was obtained as a white solid.

Step 4. Synthesis of 2-[[6-chloro-3-[(1-methyl-4-piperidyl)sulfamoyl]-4-quinolyl]amino]benzoic acid (219A): To a solution of 4,6-dichloro-N-(1-methyl-4-piperidyl)quinoline-3-sulfonamide (40 mg, 106.87 umol, 6.67e-1 eq) in ACN (2 mL) was added 2-aminobenzoic acid (21.98 mg, 160.31 umol, 1 eq) was stirred at 80° C. for 12 h. LCMS showed starting material was consumed completely and the desired product was formed. The reaction mixture was concentrated in vacuum. The crude product was purified by prep-HPLC (column: Welch Xtimate C18 100*25 mm*3 um; mobile phase: [water(0.05% HCl)-ACN]; B %: 10%-30%,8 min) Afford crude product (20 mg) The crude product was purified by prep-HPLC (column: Welch Xtimate C18 100*25 mm*3 um; mobile phase: [water(0.05% HCl)-ACN]; B %: 10%-28%,8 min) Compound 2-[[6-chloro-3-[(1-methyl-4-piperidyl)sulfamoyl]-4-quinolyl]amino]benzoic acid (15.59 mg, 29.78 umol, 18.57% yield, 97.68% purity, HCl) was obtained as a yellow solid. ¹H NMR (400 MHz, DMSO-d6+D2O, T=273+80K) δ ppm=9.12 (s, 1H) 8.10 (d, J=9.05 Hz, 1H) 8.04 (dd, J=7.95, 1.47 Hz, 1H) 7.85 (dd, J=9.05, 2.32 Hz, 1H) 7.50 (d, J=2.08 Hz, 1H) 7.33-7.42 (m, 1H) 7.17 (t, J=7.27 Hz, 1H), 6.69 (br d, J=8.19 Hz, 1H) 3.15 (br s, 4H) 2.80 (br s, 1H) 2.63 (br s, 3H) 1.54-2.03 (m, 4H). MS (M+H)⁺=475.1.

### Example 56—Synthesis of Compound 220A

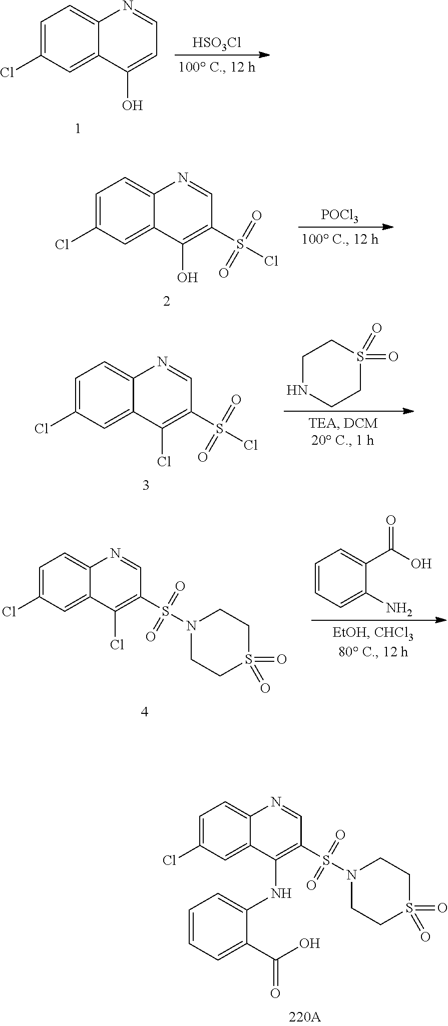

Step 1. Synthesis of 6-chloro-4-hydroxy-quinoline-3-sulfonyl chloride (2): To a solution of 6-chloroquinolin-4-ol (5.3 g, 29.51 mmol, 1 eq) in HSO₃Cl (40 mL) was stirred at 100° C. for 12 h. LCMS showed the starting material was consumed completely and the Ms of desired product was detected. The mixture was poured onto ice water(˜100 mL). Filtered, and filter cake was concentrate in vacuum. Based on TLC (Petroleum ether:Ethyl acetate=10:1, R_{f}=0.49). Compound 6-chloro-4-hydroxy-quinoline-3-sulfonyl chloride (8.0 g, 28.77 mmol, 97.48% yield) was obtained as a yellow solid. MS (M+H)+=278.1

Step 2. Synthesis of 4,6-dichloroquinoline-3-sulfonyl chloride (3): A solution of 6-chloro-4-hydroxy-quinoline-3-sulfonyl chloride (8 g, 28.77 mmol, 1 eq) in POCl₃ (50 mL) was stirred at 100° C. for 12 h. LCMS showed the starting material was consumed completely and desired Ms was detected. The reaction mixture was concentrate in vacuum. The residue was dissolved with ethyl acetate (100 mL). The mixture was poured into water (100 mL). The aqueous phase was extracted with ethyl acetate (200 mL*2). The combined organic phase was dried with anhydrous Na₂SO₄, filtered and concentrated in vacuum. The residue was purified by flash column (ISCO 40 g silica, 10-35% Ethyl acetate in Petroleum ether, gradient over 30 min). Compound 4,6-dichloroquinoline-3-sulfonyl chloride (2.2 g, 7.42 mmol, 25.79% yield) was obtained as a white solid.

Step 3. Synthesis of 4-[(4,6-dichloro-3-quinolyl)sulfonyl]-1,4-thiazinane 1,1-dioxide (220A): To a solution of 4,6-dichloroquinoline-3-sulfonyl chloride (50 mg, 168.60 umol, 1 eq) in CH₂Cl₂ (2 mL) was added 1,4-thiazinane 1,1-dioxide (22.79 mg, 168.60 umol, 1 eq) and TEA (51.18 mg, 505.80 umol, 70.40 uL, 3 eq), the mixture was stirred at 25° C. for 1 h. LCMS showed the starting material was consumed completely and the Ms of desired product was detected. The reaction mixture was concentrate in vacuum. The reaction mixture was concentrate in vacuum. Compound 4-[(4,6-dichloro-3-quinolyl)sulfonyl]-1,4-thiazinane 1,1-dioxide (50 mg, 126.49 umol, 75.02% yield) was obtained as a white solid. MS (M+H)⁺=395.0.

Step 4. Synthesis of 2-[[6-chloro-3-[(1,1-dioxo-1,4-thiazinan-4-yl)sulfonyl]-4-quinolyl]amino]benzoic acid (5): To a solution of 4-[(4,6-dichloro-3-quinolyl)sulfonyl]-1,4-thiazinane 1,1-dioxide (30 mg, 75.90 umol, 1 eq) in EtOH (0.5 mL) and CHCl₃ (0.1 mL) was added 2-aminobenzoic acid (10.41 mg, 75.90 umol, 1 eq), the mixture was stirred at 80° C. for 12 h. LCMS showed the starting material was consumed completely and the Ms of desired product was detected. The mixture was concentrate in vacuum The crude product was purified by prep-HPLC (column: Welch Xtimate C18 100*25 mm*3 um; mobile phase: [water(0.05% HCl)-ACN]; B %: 35%-65%,8 min) Compound 2-[[6-chloro-3-[(1,1-dioxo-1,4-thiazinan-4-yl)sulfonyl]-4-quinolyl]amino]benzoic acid (1.60 mg, 3.17 umol, 4.18% yield, 98.34% purity) was obtained as a yellow solid. ¹H NMR (400 MHz, DMSO-d₆) δ ppm 10.38 (br s, 1H), 9.15 (s, 1H), 8.13 (d, J=9.01 Hz, 1H), 8.01 (dd, J=7.88, 1.50 Hz, 1H), 7.91 (dd, J=9.01, 2.25 Hz, 1H), 7.59 (d, J=2.13 Hz, 1H), 7.30-7.40 (m, 1H), 7.08 (t, J=7.69 Hz, 1H), 6.69 (d, J=8.38 Hz, 1H), 3.63-3.69 (m, 4H), 3.12-3.24 (m, 2H), 2.95-3.11 (m, 2H). MS (M+H)⁺=495.9.

### Example 57—Synthesis of Compound 221A

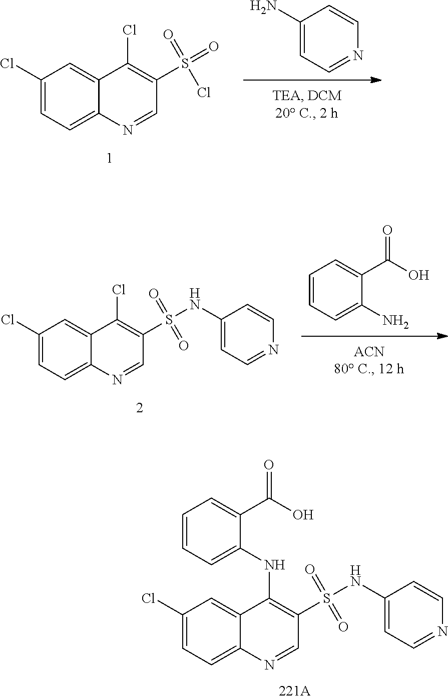

Step 1. Synthesis of 4,6-dichloro-N-(4-pyridyl)quinoline-3-sulfonamide (2): To a stirred solution of 4,6-dichloroquinoline-3-sulfonyl chloride (60 mg, 202.32 umol, 1 eq) in CHCl₃ (1 mL) was added pyridin-4-amine (19.04 mg, 202.32 umol, 34.00 uL, 1 eq) TEA (61.42 mg, 606.97 umol, 84.48 uL, 3 eq) the mixture was stirred at 25° C. for 2 h. LCMS showed the starting material was consumed completely and desired MS was detected. The reaction mixture was concentrate in vacuum. The residue was purified by prep-HPLC (column: Phenomenex luna C18 80*40 mm*3 um; mobile phase: [water(0.04% HCl)-ACN]; B %: 22%-42%,7 min). Compound 4,6-dichloro-N-(4-pyridyl)quinoline-3-sulfonamide (10 mg, crude, HCl) was obtained as a yellow solid. MS (M+H)*=354.1.

Step 2. Synthesis of 2-[[6-chloro-3-(4-pyridylsulfamoyl)-4-quinolyl]amino]benzoic acid (221A): A solution of 4,6-dichloro-N-(4-pyridyl)quinoline-3-sulfonamide (10 mg, 28.23 umol, 1 eq) 2-aminobenzoic acid (3.87 mg, 28.23 umol, 1 eq) in ACN (0.5 mL) was stirred at 80° C. for 2 h. LCMS showed the Ms of desired product was detected. The reaction mixture was concentrate in vacuum. The residue was purified by prep-HPLC (column: Phenomenex luna C18 80*40 mm*3 um; mobile phase: [water(0.04% HCl)-ACN]; B %: 15%-43%,7 min). Compound 2-[[6-chloro-3-(4-pyridylsulfamoyl)-4-quinolyl]amino]benzoic acid (0.34 mg, 6.18e-1 umol, 2.19% yield, 89.30% purity, HCl) was obtained as a yellow solid. ¹H NMR (400 MHz, DMSO-d₆) δ=9.27 (s, 1H), 8.08 (d, J=8.9 Hz, 1H), 8.03-7.95 (m, 3H), 7.82 (dd, J=2.2, 9.0 Hz, 1H), 7.50 (d, J=2.1 Hz, 1H), 7.27-7.20 (m, 1H), 7.05-6.97 (m, 3H), 6.39 (d, J=8.4 Hz, 1H). MS (M+H)⁺=455.0.

### Example 58—Synthesis of Compound 226A

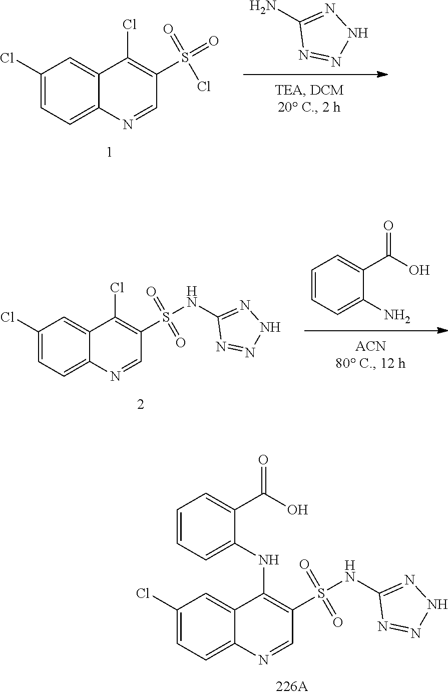 [SMILES: O=S(=O)(Cl)c1cnc2ccc(Cl)cc2c1Cl ; Nc1nn[nH]n1 ; O=S(=O)(Nc1nn[nH]n1)c1cnc2ccc(Cl)cc2c1Cl ; Nc1ccccc1C(=O)O ; O=C(O)c1ccccc1Nc1c(S(=O)(=O)Nc2nn[nH]n2)cnc2ccc(Cl)cc12]

Step 1. Synthesis of 4,6-dichloro-N-(2H-tetrazol-5-yl)quinoline-3-sulfonamide (2): To a solution of 2H-tetrazol-5-amine (43.03 mg, 505.80 umol, 1 eq) in THF (1 mL) was added NaH (30.35 mg, 758.71 umol, 60% purity, 1.5 eq) at 0° C. for 0.5 h under N₂. 4,6-dichloroquinoline-3-sulfonyl chloride (150 mg, 505.80 umol, 1 eq) was added, the mixture was stirred at 20° C. for 2 h. LCMS showed the starting material was consumed completely and desired MS was detected. The reaction mixture was added dropwise to 2N HCl (5 mL). Filtered, and filter cake was concentrate in vacuum. Compound 4,6-dichloro-N-(2H-tetrazol-5-yl)quinoline-3-sulfonamide (120 mg, crude) was obtained as a yellow solid. MS (M+H)⁺=345.0.

Step 2. Synthesis of 2-[[6-chloro-3-(2H-tetrazol-5-ylsulfamoyl)-4-quinolyl]amino]benzoic acid (226A): A solution of 4,6-dichloro-N-(2H-tetrazol-5-yl)quinoline-3-sulfonamide (80 mg, 231.77 umol, 1 eq), 2-aminobenzoic acid (31.78 mg, 231.77 umol, 1 eq) in ACN (1 mL) was stirred at 80° C. for 12 h. LCMS showed the starting material was consumed completely and desired MS was detected. The reaction mixture was concentrate in vacuum. The residue was purified by prep-HPLC (column: Welch Xtimate C18 100*25 mm*3 um; mobile phase: [water(0.05% HCl)-ACN]; B %: 20%-50%,8 min). Afford 30 mg crude product. The crude product was purified by prep-HPLC (column: Phenomenex luna C18 80*40 mm*3 um; mobile phase: [water(0.1% TFA)-ACN]; B %: 35%-45%,7 min). Compound 2-[[6-chloro-3-(2H-tetrazol-5-ylsulfamoyl)-4-quinolyl]amino]benzoic acid (4.07 mg, 7.27 umol, 3.14% yield, 100% purity, TFA) was obtained as a yellow solid. ¹H NMR (400 MHz, DMSO-d₆) δ=10.17 (s, 1H), 9.32 (s, 1H), 8.96 (s, 1H), 8.14 (d, J=9.0 Hz, 1H), 7.99 (dd, J=1.5, 8.0 Hz, 1H), 7.93 (s, 1H), 7.89 (dd, J=2.3, 9.0 Hz, 1H), 7.63 (d, J=2.2 Hz, 1H), 7.35-7.19 (m, 1H), 7.05-6.93 (m, 1H), 6.39 (d, J=8.3 Hz, 1H). MS (M+H)⁺=446.0.

### Example 59—Synthesis of Compound 231A

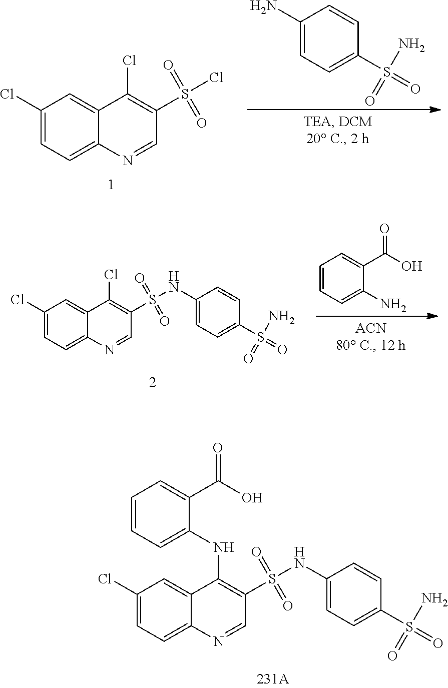 [SMILES: O=S(=O)(Cl)c1cnc2ccc(Cl)cc2c1Cl ; Nc1ccc(S(N)(=O)=O)cc1 ; NS(=O)(=O)c1ccc(NS(=O)(=O)c2cnc3ccc(Cl)cc3c2Cl)cc1 ; Nc1ccccc1C(=O)O ; NS(=O)(=O)c1ccc(NS(=O)(=O)c2cnc3ccc(Cl)cc3c2Nc2ccccc2C(=O)O)cc1]

Step 1. Synthesis of 4,6-dichloro-N-(4-sulfamoylphenyl)quinoline-3-sulfonamide (2): To a solution of 4-aminobenzenesulfonamide (87.10 mg, 505.80 umol, 87.19 uL, 1 eq) in THF (1 mL) was added NaH (30.35 mg, 758.71 umol, 60% purity, 1.5 eq) at 0° C. for 0.5 h under N₂. 4,6-dichloroquinoline-3-sulfonyl chloride (150 mg, 505.80 umol, 1 eq) was added, the mixture was stirred at 20° C. for 2 h. LCMS showed the starting material was consumed completely and desired MS was detected. The reaction mixture was added dropwise into 2N HCl (5 mL). Filtered, and filter cake was concentrate in vacuum. Compound 4,6-dichloro-N-(4-sulfamoylphenyl)quinoline-3-sulfonamide (100 mg, crude) was obtained as a yellow solid. MS (M+H)⁺=432.2.

Step 2. Synthesis of 2-[[6-chloro-3-[(4-sulfamoylphenyl)sulfamoyl]-4-quinolyl]amino]benzoic acid (231A): A solution of 4,6-dichloro-N-(4-sulfamoylphenyl)quinoline-3-sulfonamide (50 mg, 115.66 umol, 1 eq),2-aminobenzoic acid (15.86 mg, 115.66 umol, 1 eq) in ACN (2 mL) was stirred at 80° C. for 12 h. LCMS showed the starting material was consumed completely and desired MS was detected. The reaction mixture was concentrate in vacuum. The residue was purified by prep-HPLC (column: Welch Xtimate C18 150*25 mm*5 um; mobile phase: [water(0.05% HCl)-ACN]; B %: 5%-30%,8 min). Compound 2-[[6-chloro-3-[(4-sulfamoylphenyl)sulfamoyl]-4-quinolyl]amino]benzoic acid (2.07 mg, 3.39 umol, 2.93% yield, 93.23% purity, HCl) was obtained as a yellow solid. ¹H NMR (400 MHz, DMSO-d₆) δ=10.71-10.35 (m, 1H), 9.00 (s, 1H), 8.12-8.00 (m, 2H), 7.93 (dd, J=1.9, 8.9 Hz, 1H), 7.43(br t, J=8.1 Hz, 1H), 7.36-7.23 (m, 4H), 6.75 (d, J=8.0 Hz, 1H), 6.45 (br d, J=8.4 Hz, 2H). MS (M+H)⁺=533.0.

### Example 50A—Synthesis of Compound 232A

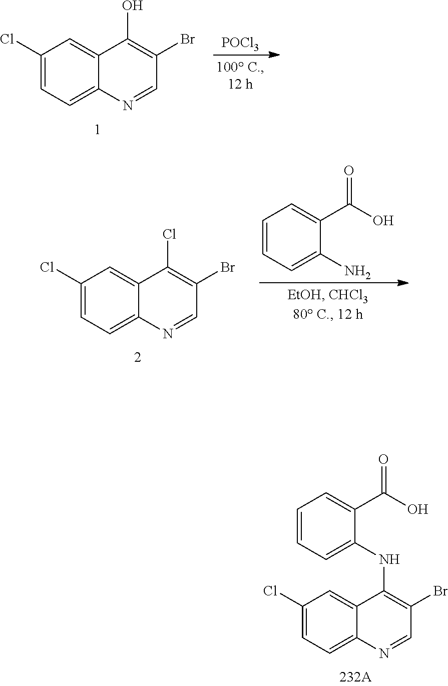

Step 1. Synthesis of 3-bromo-4,6-dichloro-quinoline (2): To a solution of 3-bromo-6-chloro-quinolin-4-ol (4 g, 15.47 mmol, 1 eq) in POCl₃ (50 mL) was stirred at 100° C. for 12 h. TLC (Petroleum ether/Ethyl acetate=5:1, R_{f}=0.50) showed starting material was consumed completely and new spot was formed. The reaction mixture was concentrate in vacuum. The residue was dissolved with ethyl acetate (100 mL). The mixture was poured into water (50 mL). The aqueous phase was extracted with ethyl acetate (100 mL*2). The combined organic phase was dried with anhydrous Na₂SO₄, filtered and concentrated in vacuum. The residue was purified by flash column (ISCO 50 g silica, 5-15% Ethyl acetate in Petroleum ether, gradient over 15 min). Compound 3-bromo-4,6-dichloro-quinoline (2.97 g, 10.72 mmol, 69.32% yield) was obtained as a white solid. MS (M+H)¹=278.0

Step 2. Synthesis of 2-[(3-bromo-6-chloro-4-quinolyl)amino]benzoic acid (232A): To a solution of 3-bromo-4,6-dichloro-quinoline (100 mg, 361.08 umol, 1 eq) in EtOH (5 mL) and CHCl₃ (1 mL) was added 2-aminobenzoic acid (49.52 mg, 361.08 umol, 1 eq), the mixture was stirred at 80° C. for 12 h. LCMS showed the starting material was consumed completely and the MS of desired product was detected. The reaction mixture was concentrate in vacuum. The crude product was purified by prep-HPLC (column: Phenomenex luna C18 80*40 mm*3 um; mobile phase: [water(0.04% HCl)-ACN]; B %: 40%-70%,7 min) Compound 2-[(3-bromo-6-chloro-4-quinolyl)amino]benzoic acid (12.40 mg, 29.95 umol, 8.29% yield, 100% purity, HCl) was obtained as a yellow solid. ¹H NMR (400 MHz, DMSO-d₆) δ ppm 9.98 (s, 1H), 9.09 (s, 1H), 8.06-8.19 (m, 1H), 7.98 (dd, J=8.00, 1.50 Hz, 1H), 7.80-7.91 (m, 2H), 7.25-7.39 (m, 1H), 6.94 (t, J=7.50 Hz, 1H), 6.39 (d, J=8.25 Hz, 1H). MS (M+H)⁺=378.9.

### Example 51A—Synthesis of Compound 233A

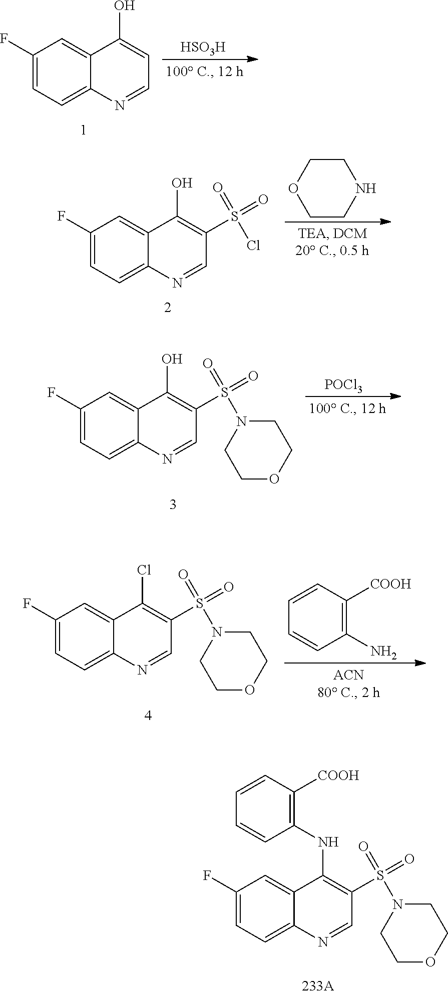

Step 1. Synthesis of 6-fluoro-4-hydroxy-quinoline-3-sulfonyl chloride (2): To a solution of 6-fluoroquinolin-4-ol (1.15 g, 7.05 mmol, 1 eq) in HSO₃Cl (10 mL) was stirred at 100° C. for 12 h. TLC (Petroleum ether/Ethyl acetate=3:1, R_{f}=0.20) showed starting material was consumed completely and new spot was formed. The reaction mixture was concentrated in vacuum. The residue was dissolved with ethyl acetate (100 mL). The mixture was poured into water (100 mL). The aqueous phase was extracted with ethyl acetate (200 mL*2). The combined organic phase was dried with anhydrous Na₂SO₄, filtered and concentrated in vacuum. The residue was purified by flash column (ISCO 40 g silica, 30-50% Ethyl acetate in Petroleum ether, gradient over 15 min). Compound 6-fluoro-4-hydroxy-quinoline-3-sulfonyl chloride (1.9 g, crude) was obtained as a white solid.

Step 2. Synthesis of 6-fluoro-3-morpholinosulfonyl-quinolin-4-ol (3): To a solution of 6-fluoro-4-hydroxy-quinoline-3-sulfonyl chloride (1.9 g, 7.26 mmol, 1 eq) in CHCl₃ (10 mL) was added TEA (2.20 g, 21.78 mmol, 3.03 mL, 3 eq) and morpholine (632.61 mg, 7.26 mmol, 639.00 uL, 1 eq), the mixture was stirred at 20° C. for 0.5 h. LCMS showed the starting material was consumed completely and the MS of desired product was detected. The reaction mixture was concentrated in vacuum. No purification, used for next step. Compound 6-fluoro-3-morpholinosulfonyl-quinolin-4-ol (1.2 g, 3.84 mmol, 52.91% yield) was obtained as a white solid. MS (M+H)⁺=313.2

Step 3. Synthesis of 4-[(4-chloro-6-fluoro-3-quinolyl)sulfonyl]morpholine (4): To a solution of 6-fluoro-3-morpholinosulfonyl-quinolin-4-ol (1 g, 3.20 mmol, 1 eq) in POCl₃ (10 mL) was stirred at 100° C. for 12 h. LCMS showed starting material was consumed completely and the MS of desired product was detected. TLC (PE:EtOAc=3:1, R_{f}=0.25) showed starting material was consumed completely and new spot was formed. The reaction mixture was concentrated in vacuum. The residue was dissolved with ethyl acetate (100 mL). The mixture was poured into water (100 mL). The aqueous phase was extracted with ethyl acetate (200 mL*2). The combined organic phase was dried with anhydrous Na₂SO₄, filtered and concentrated in vacuum. The residue was purified by flash column (ISCO 10 g silica, 70-90% Ethyl acetate in Petroleum ether, gradient over 30 min). Compound 4-[(4-chloro-6-fluoro-3-quinolyl) sulfonyl]morpholine (1.02 g, 3.08 mmol, 96.31% yield) was obtained as a white solid. MS (M+H)⁺=331.1

Step 4. Synthesis of 2-[(6-fluoro-3-morpholinosulfonyl-4-quinolyl)amino]benzoic acid (233A): A solution of 4-[(4-chloro-6-fluoro-3-quinolyl)sulfonyl]morpholine (100 mg, 302.33 umol, 1 eq) in ACN (1.5 mL) was added 2-aminobenzoic acid (41.46 mg, 302.33 umol, 1 eq), the mixture was stirred at 80° C. for 2 h. LCMS showed the starting material was consumed completely and the MS of desired product was detected. The reaction mixture was concentrated in vacuum. The crude product was purified by prep-HPLC (column: Welch Xtimate C18 100*25 mm*3 um; mobile phase: [water (0.04% HCl)-ACN]; B %: 20%-50%,8 min) Compound 2-[(6-fluoro-3-morpholinosulfonyl-4-quinolyl)amino]benzoic acid (43.51 mg, 91.25 umol, 30.18% yield, 98.13% purity, HCl) was obtained as a yellow solid. ¹H NMR (400 MHz, DMSO-d₆) δ ppm 10.45 (br s, 1H), 9.08 (s, 1H), 8.21 (dd, J=9.23, 5.44 Hz, 1H), 8.00 (dd, J=7.82, 1.34 Hz, 1H), 7.79-7.88 (m, 1H), 7.31-7.40 (m, 1H), 7.26 (dd, J=10.15, 2.69 Hz, 1H), 7.07 (t, J=7.52 Hz, 1H), 6.67 (br d, J=8.19 Hz, 1H), 3.41-3.52 (m, 2H), 3 3.26-3.39 (m, 2H), 3.04 (dddd, J=15.21, 12.21, 8.83, 3.00 Hz, 4H). MS (M+H)⁺=432.1.

### Example 52A—Synthesis of 234A

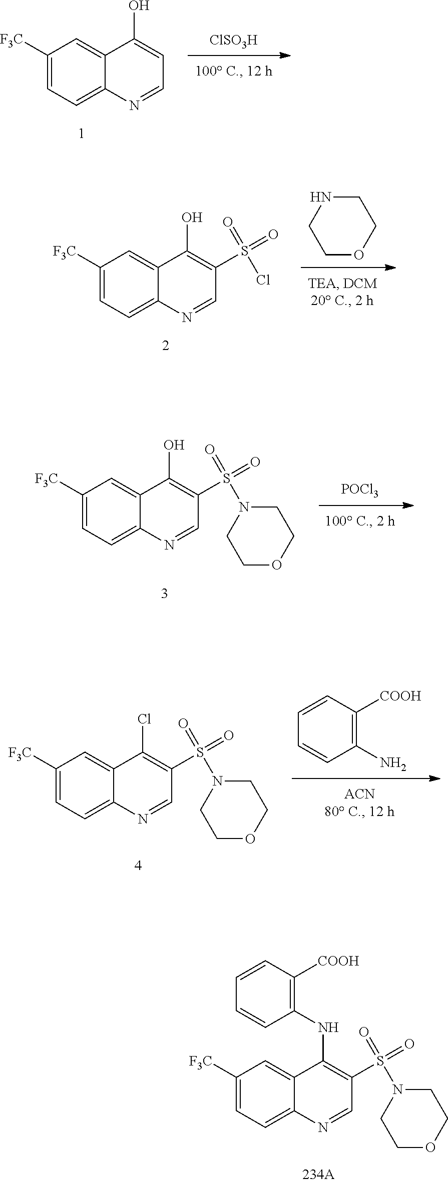

Step 1. Synthesis of 4-hydroxy-6-(trifluoromethyl)quinoline-3-sulfonyl chloride (2): To a solution of 6-(trifluoromethyl)quinolin-4-ol (1 g, 4.69 mmol, 1 eq) in HSO₃Cl (5 mL) was stirred at 100° C. for 12 h. LCMS showed starting material was consumed completely and the MS of desired product was not detected. The reaction mixture was concentrated in vacuum. Compound 4-hydroxy-6-(trifluoromethyl)quinoline-3-sulfonyl chloride (1.7 g, crude) was obtained as a white solid. MS (M+H)⁺=312.2.

Step 2. Synthesis of 3-morpholinosulfonyl-6-(trifluoromethyl)quinolin-4-ol (3): To a solution of 4-hydroxy-6-(trifluoromethyl)quinoline-3-sulfonyl chloride (1.7 g, 5.45 mmol, 1 eq) in CHCl₃ (5 mL) was added TEA (1.66 g, 16.36 mmol, 2.28 mL, 3 eq) and morpholine (475.20 mg, 5.45 mmol, 480.00 uL, 1 eq), the mixture was stirred at 20° C. for 0.5 h. LCMS showed starting material was consumed completely and the MS of desired product was detected. The reaction mixture was concentrated in vacuum. The crude product was purified by prep-HPLC (column: Phenomenex luna C18 250*50 mm*10 um; mobile phase: [water(0.04% HCl)-ACN]; B %: 10%-50%,10 min) Compound 3-morpholinosulfonyl-6-(trifluoromethyl)quinolin-4-ol (31 mg. 85.56 umol, 1.57% yield) was obtained as a white solid. MS (M+H)⁺=363.3

Step 3. Synthesis of 4-[[4-chloro-6-(trifluoromethyl)-3-quinolyl]sulfonyl]morpholine (4): A solution of 3-morpholinosulfonyl-6-(trifluoromethyl)quinolin-4-ol (10 mg, 27.60 umol, 1 eq) in POCl₃ (0.3 mL) was stirred at 100° C. for 12 h under N₂. LCMS showed starting material was consumed completely and the MS of desired product was detected. The reaction mixture was concentrated in vacuum. Compound 4-[[4-chloro-6-(trifluoromethyl)-3-quinolyl]sulfonyl]morpholine (10 mg, 26.26 umol, 95.15% yield) was obtained as a white solid.

Step 4. Synthesis of 2-[[3-morpholinosulfonyl-6-(trifluoromethyl)-4-quinolyl]amino]benzoic acid (234A): To a solution of 4-[[4-chloro-6-(trifluoromethyl)-3-quinolyl]sulfonyl]morpholine (10 mg, 26.26 umol, 1 eq) in ACN (0.5 mL) was added 2-aminobenzoic acid (3.60 mg, 26.26 umol, 1 eq), the reaction mixture was stirred at 80° C. for 12 h. LCMS showed starting material was consumed completely and the MS desired product was detected. The reaction mixture was concentrated in vacuum. The crude product was purified by pre-HPLC (column: Welch Xtimate C18 100*25 mm*3 um; mobile phase: [water(0.04% HCl)-ACN]; B %: 25%-55%,8 min) Compound 2-[[3-morpholinosulfonyl-6-(trifluoromethyl)-4-quinolyl]amino]benzoic acid (2.38 mg, 4.43 umol, 16.88% yield, 96.49% purity, HCl) was obtained as a yellow solid. ¹H NMR (400 MHz, DMSO-d₆) δ ppm 10.52 (br s, 1H), 9.15 (s, 1H), 8.26 (br d, J=8.80 Hz, 1H), 8.10 (br d, J=7.33 Hz, 1H), 7.97-8.04 (m, 1H), 7.90 (s, 1H), 7.33 (t, J=7.09 Hz, 1H), 7.10 (t, J=7.52 Hz, 1H), 6.79 (d, J=8.19 Hz, 1H), 3.49-3.57 (m, 4H), 2.94-3.16 (m, 4H). MS (M+H)⁺=482.0.

### Example 53A—Synthesis of Compound 235A

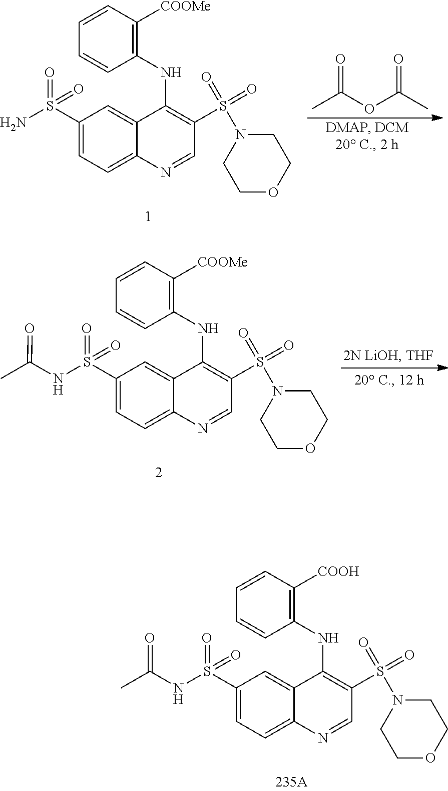

Step 1. Synthesis of methyl 2-[[6-(acetylsulfamoyl)-3-morpholinosulfonyl-4-quinolyl]amino]benzoate (2): To a stirred solution of methyl 2-[(3-morpholinosulfonyl-6-sulfamoyl-4-quinolyl)amino]benzoate (60 mg, 118.45 umol, 1 eq), acetyl acetate (13.30 mg, 130.29 umol, 12.20 uL, 1.1 eq) in THF (2 mL) was added DMAP (28.94 mg, 236.90 umol, 2 eq), the mixture was purged with N₂ for 3 times, and stirred at 20° C. for 2 h. LCMS showed the starting material was consumed completely and the MS of desired product was detected. The reaction mixture was poured into water (10 mL). The aqueous phase was extracted with ethyl acetate (10 mL*2). The combined organic phase was dried with anhydrous Na₂SO₄, filtered and concentrated in vacuum. Compound methyl 2-[[6-(acetylsulfamoyl)-3-morpholinosulfonyl-4-quinolyl]amino]benzoate (40 mg, 72.91 umol, 61.56% yield) was obtained as a yellow oil. MS (M+H)⁺=549.2

Step 2. Synthesis of methyl 2-[[6-(acetylsulfamoyl)-3-morpholinosulfonyl-4-quinolyl]amino]benzoic acid (235A): To a solution of methyl 2-[[6-(acetylsulfamoyl)-3-morpholinosulfonyl-4-quinolyl]amino]benzoate (40 mg, 72.91 umol, 1 eq) in THF (1 mL) was added LiOH·H₂O (6.12 mg, 145.83 umol, 2 eq), the reaction mixture was stirred at 20° C. for 12 h. LCMS showed the starting material was consumed completely and the MS of desired product was detected. The reaction mixture was concentrated in vacuum. The crude product was purified by prep-HPLC (column: Phenomenex luna C18 80*40 mm*3 um; mobile phase: [water (0.04% HCl)-ACN]; B %: 17%-53%,7 min) Compound 2-[[6-(acetylsulfamoyl)-3-morpholinosulfonyl-4-quinolyl]amino]benzoic acid (10.62 mg, 17.70 umol, 24.27% yield, 95.16% purity, HCl) was obtained as a yellow solid. ¹H NMR (400 MHz, DMSO-d₆) δ ppm 12.20 (s, 1H), 10.54 (br s, 1H), 9.17 (s, 1H), 8.18-8.31 (m, 3H), 8.02 (d, J=7.82 Hz, 1H), 7.33 (t, J=7.82 Hz, 1H), 7.27-7.38 (m, 1H), 7.11 (t, J=7.46 Hz, 1H), 3.47-3.55 (m, 2H), 3.37-3.42 (m, 2H), 3.01-3.13 (m, 4H), 1.79-1.84 (s, 3H). MS (M+H)⁺=535.0.

### Example 54A—Synthesis of Compound 236A

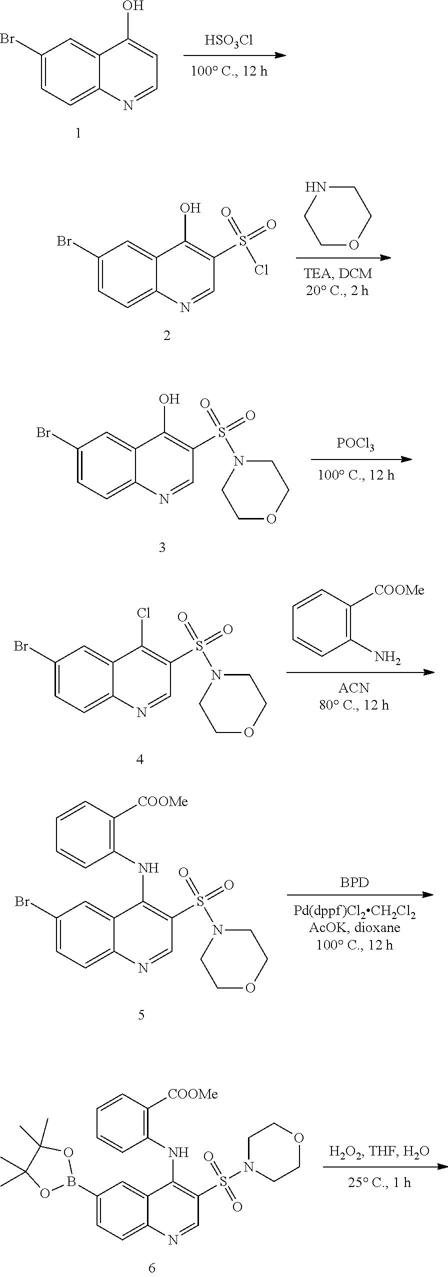

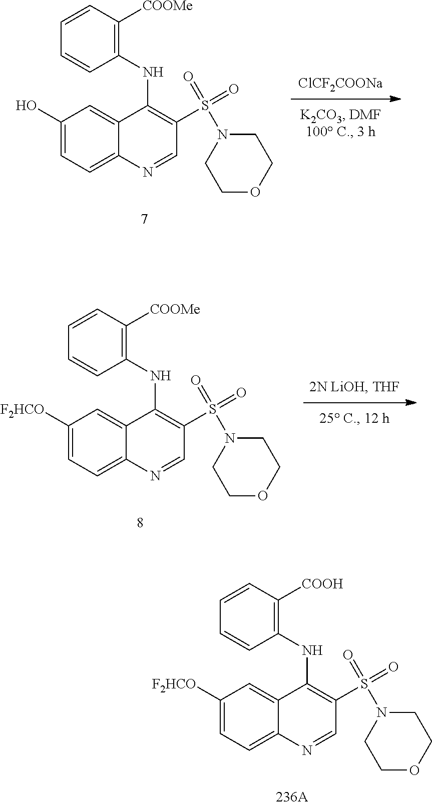

Step 1. Synthesis of 6-bromo-4-hydroxy-quinoline-3-sulfonyl chloride (2): A solution of 6-bromoquinolin-4-ol (2 g, 8.93 mmol, 1 eq) in HSO₃Cl (15 mL) was stirred at 100° C. for 12 h. LCMS showed the starting material was consumed completely and desired Ms w as detected. The reaction mixture was cooled to 25° C. Then poured into ice water. Filtered and filter cake was concentrate in vacuum. Compound 6-bromo-4-hydroxy-quinoline-3-sulfonyl chloride (2.5 g, crude) was obtained as a white solid.

Step 2. Synthesis of 6-bromo-3-morpholinosulfonyl-quinolin-4-ol (3): To a stirred solution of 6-bromo-4-hydroxy-quinoline-3-sulfonyl chloride (2.5 g, 7.75 mmol, 1 eq) in D CM (30 mL) was added TEA (2.35 g, 23.25 mmol, 3.24 mL, 3 eq) and morpholine (1.01 g, 11.63 mmol, 1.02 mL, 1.5 eq) at 25° C., then the mixture was stirred at 25° C. for 2 h. LCMS showed the starting material was consumed completely and desired Ms was detected. The reaction mixture was concentrate in vacuum. Compound 6-bromo-3-morpholinosulfonyl-quinolin-4-ol (4 g, crude) was obtained as a yellow oil. MS (M+H)⁺=375.1.

Step 3. Synthesis of 4-[(6-bromo-4-chloro-3-quinolyl)sulfonyl]morpholine (4): A solution of 6-bromo-3-morpholinosulfonyl-quinolin-4-ol (3 g, 8.04 mmol, 1 eq) in POCl₃ (30 mL) was stirred at 100° C. for 12 h. LCMS showed the starting material was consumed completely and desired MS was detected. The reaction mixture was concentrate in vacuum. Then the mixture was dissolved in ethyl acetate (20 mL), and dropwise into water (50 mL). The aqueous phase was extracted with ethyl acetate (50 mL*2). The combined organic phase was dried with anhydrous Na₂SO₄, filtered and concentrated in vacuum. The crude product was purified by flash column (ISCO 10 g silica, 60-70% ethyl acetate in petroleum ether, gradient over 20 min). Based on TLC (Petroleum ether:Ethyl acetate=1/1, R_{f}=0.32). Compound 4-[(6-bromo-4-chloro-3-quinolyl)sulfonyl]morpholine (1.5 g, crude) was obtained as a white solid. MS (M+H)⁺=393.0.

Step 4. Synthesis of methyl 2-[(6-bromo-3-morpholinosulfonyl-4-quinolyl)amino]benzoate (5): A solution of 4-[(6-bromo-4-chloro-3-quinolyl)sulfonyl]morpholine (1 g, 2.55 mmol, 1 eq) and methyl 2-aminobenzoate (385.95 mg, 2.55 mmol, 329.87 uL, 1 eq) in ACN (15 mL) was stirred at 80° C. for 12 h. LCMS showed the starting material was consumed completely and desired product was detected. The reaction mixture was filtered, and filter caked was concentrate in vacuum. Compound methyl 2-[(6-bromo-3-morpholinosulfonyl-4-quinolyl)amino]benzoate (1.3 g, crude) was obtained as a yellow solid. MS (M+H)⁺=508.0.

Step 5. Synthesis of methyl 2-[[3-morpholinosulfonyl-6-(4,4,5,5-tetramethyl-1,3,2-dioxaborolan-2-yl)-4-quinolyl]amino]benzoate (6): To a stirred solution of methyl 2-[(6-bromo-3-morpholinosulfonyl-4-quinolyl)amino]benzoate (1 g, 1.97 mmol, 1 eq) in DIOXANE (10 mL) was added BPD (601.78 mg, 2.37 mmol, 1.2 eq) Pd(dppf)Cl₂·CH₂Cl₂ (161.27 mg, 197.48 umol, 0.1 eq) and AcOK (581.45 mg, 5.92 mmol, 3 eq) at 25° C., then the mixture was purged with N₂ for 3 times, and stirred at 110° C. for 12 h. LCMS showed the starting material was consumed completely and desired product was detected. The reaction mixture was concentrate in vacuum. The crude product was purified by flash column (ISCO 10 g silica, 80-90% ethyl acetate in petroleum ether, gradient over 20 min). Based on TLC (Petroleum ether:Ethyl acetate=0/1, R_{f}=0.14). Compound methyl 2-[[3-morpholinosulfonyl-6-(4,4,5,5-tetramethyl-1,3,2-dioxaborolan-2-yl)-4-quinolyl]amino]benzoate (400 mg, 722.76 umol, 36.60% yield) was obtained as a yellow solid.

Step 6. Synthesis of methyl 2-[(6-hydroxy-3-morpholinosulfonyl-4-quinolyl)amino]benzoate (7): To a stirred solution of methyl 2-[[3-morpholinosulfonyl-6-(4,4,5,5-tetrame-thyl-1,3,2-dioxaborolan-2-yl)-4-quinolyl]amino]benzoate (300 mg, 542.07 umol, 1 eq) in Water (5 mL) and THF (5 mL) was added H₂O₂ (0.54 g, 4.76 mmol, 457.63 uL, 30% purity, 8.79 eq) at 25° C., then the mixture was stirred at 25° C. for 12 h. LCMS showed the starting material was consumed completely and desired product was detected. The reaction mixture was poured into water (10 mL). The aqueous phase was extracted with ethyl acetate (10 mL*3). The combined organic phase was dried with anhydrous Na₂SO₄, filtered and concentrated in vacuum. The residue was purified by flash column (ISCO 10 g silica, 60-65% Ethyl acetate in Petroleum ether, gradient over 15 min). Based on TLC (Petroleum ether:Ethyl acetate=1/1, R_{f}=0.46). Compound methyl 2-[(6-hydroxy-3-morpholinosulfonyl-4-quinolyl)amino]benzoate (160 mg, 360.79 umol, 66.56% yield) was obtained as a yellow solid. MS (M+H)⁺=444.0

Step 7. Synthesis of methyl 2-[[6-(difluoromethoxy)-3-morpholinosulfonyl-4-quinolyl]amino]benzoate (8): To a stirred solution of methyl 2-[(6-hydroxy-3-morpholinosulf-onyl-4-quinolyl)amino]benzoate (120 mg, 270.59 umol, 1 eq) in DMF (1 mL) was added K₂CO₃ (37.40 mg, 270.59 umol, 1 eq) and sodium; 2-chloro-2,2-difluoro-acetate (41.25 mg, 27.59 umol, 1 eq) at 25° C., the mixture was purged with N₂ for 3 times, and stirred at 100° C. for 2 h. LCMS showed the starting material was consumed completely and desired product was detected. The reaction mixture was filtered, and filtrate was purified directly. The filtrate was purified by prep-HPLC (column: Phenomenex luna C18 80*40 mm*3 um; mobile phase: [water(0.04% HCl)-ACN]; B %: 36%-54%,7 min). Compound methyl 2-[[6-(difluorometho-xy)-3-morpholinosulfonyl-4-quinolyl]amino]benzoate (40 mg, 81.06 umol, 29.96% yield) w as obtained as a yellow solid. MS (M+H)⁺=494.1

Step 8. Synthesis of 2-[[6-(difluoromethoxy)-3-morpholinosulfonyl-4-quinolyl]amino]benzoic acid (236A): To a stirred solution of methyl 2-[[6-(difluoromethoxy)-3-morpholinosulfonyl-4-quinolyl]amino]benzoate (20 mg, 40.53 umol, 1 eq) in THF (0.2 mL) and MeOH (0.2 mL) was added LiOH·H₂O (2 M, 40.53 uL, 2 eq) at 25° C., then the mixture was stirred at 25° C. for 4 h. LCMS showed the starting material was consumed completely and desired product was detected. The reaction mixture was adjusted pH-4 by adding 2N HCl. The residue was purified by prep-HPLC (column: Phenomenex luna C18 80*40 mm*3 um; mobile phase: [water(0.04% HCl)-ACN]; B %: 22%-50%,7 min). Compound 2-[[6-(difluoromethoxy)-3-morpholinosulfonyl-4-quinolyl]amino]benzoic acid (5.4 mg, 10.47 umol, 25.83% yield, 100% purity, HCl) was obtained as a yellow solid. ¹H NMR (400 MHz, DMSO-d₆) δ=10.45 (br s, 1H), 9.07 (s, 1H), 8.18 (d, J=9.3 Hz, 1H), 8.00 (dd, J=1.4, 7.8 Hz, 1H), 7.74 (dd, J=2.1, 9.1 Hz, 1H), 7.37-7.30 (m, 1H), 7.30-6.91 (m, 3H), 6.66 (br t, J=6.6 Hz, 1H), 3.41-3.31 (m, 4H), 3.12-2.98 (m, 4H). MS (M+H)⁺=480.0.

### Example 55A—Synthesis of Compound 237A

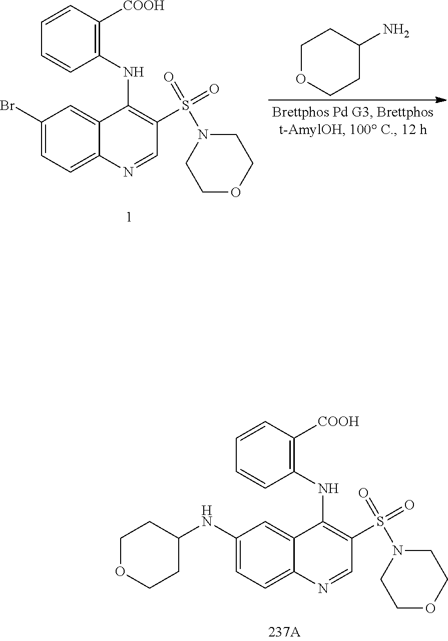 [SMILES: O=C(O)c1ccccc1Nc1c(S(=O)(=O)N2CCOCC2)cnc2ccc(Br)cc12 ; NC1CCOCC1 ; O=C(O)c1ccccc1Nc1c(S(=O)(=O)N2CCOCC2)cnc2ccc(NC3CCOCC3)cc12]

To a stirred solution of 2-[(6-bromo-3-morpholinosulfonyl-4-quinolyl)amino]benzoic acid (40 mg, 81.24 umol, 1 eq) in 2-methylbutan-2-ol (1 mL) was added tetrahydropyran-4-amine (8.22 mg, 81.24 umol, 1 eq), BrettPhos Pd G3 (7.36 mg, 8.12 umol, 0.1 eq), BRETTPHOS (4.36 mg, 8.12 umol, 0.1 eq) and t-BuONa (23.42 mg, 243.72 umol, 3 eq) the mixture was bubbled with N₂ for 1 minute, and stirred at 100° C. for 12 h. LCMS showed the starting material was consumed completely and desired MS was detected. The reaction mixture was filtered, and filtrate was purified by prep-HPLC (column: Phenomenex luna C18 80*40 mm*3 um; mobile phase: [water(0.04% HCl)-ACN]; B %: 16%-34%,7 min). Compound 2-[[3-morpholinosulfonyl-6-(tetrahydropyran-4-ylamino)-4-quinolyl]amino]benzoic acid (12.66 mg, 21.69 umol, 26.69% yield, 94.05% purity, HCl) was obtained as a yellow solid. ¹H NMR (400 MHz, DMSO-d₆) δ=8.80 (s, 1H), 8.03 (dd, J=1.4, 7.9 Hz, 1H), 7.84 (d, J=9.3 Hz, 1H), 7.47-7.34 (m, 2H), 7.20 (t, J=7.6 Hz, 1H), 6.78 (d, J=8.2 Hz, 1H), 6.14 (d, J=2.3 Hz, 1H), 3.84-3.75 (m, 1H), 3.73-3.64 (m, 1H), 3.57-3.48 (m, 2H), 3.46-3.37 (m, 2H), 3.22-3.02 (m, 5H), 2.97-2.87 (m, 1H), 2.78-2.70 (m, 1H), 1.59 (br d, J=13.0 Hz, 1H), 1.42-1.22 (m, 2H), 1.11-0.99 (m, 1H). MS (M+H)¹=513.2.

### Example 56A—Synthesis of Compound 238A

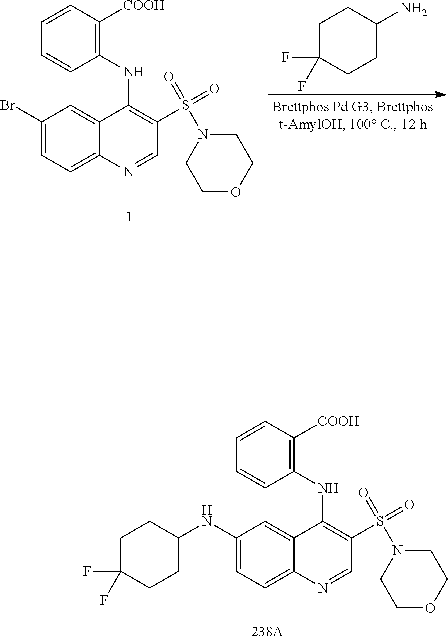 [SMILES: O=C(O)c1ccccc1Nc1c(S(=O)(=O)N2CCOCC2)cnc2ccc(Br)cc12 ; NC1CCC(F)(F)CC1 ; O=C(O)c1ccccc1Nc1c(S(=O)(=O)N2CCOCC2)cnc2ccc(NC3CCC(F)(F)CC3)cc12]

To a stirred solution of 2-[(6-bromo-3-morpholinosulfonyl-4-quinolyl)amino]benzoic acid (20 mg, 40.62 umol, 1 eq) in THF (1 mL) was added 4,4-difluorocyclohexanamine (5.49 mg, 40.62 umol, 1 eq), BRETTPHOS (2.18 mg, 4.06 umol, 0.1 eq), BrettPhos Pd G3 (3.68 mg, 4.06 umol, 0.1 eq) and t-BuONa (11.71 mg, 121.86 umol, 3 eq) the mixture was bubbled with N₂ for 1 minute, and stirred at 80° C. for 12 h. LCMS showed the starting material was consumed completely and desired Ms was detected. The reaction mixture was filtered, and filtrate was purified by prep-HPLC (column: Phenomenex Gemini-NX C18 75*30 mm*3 um; mobile phase:[water(0.04% HCl)-ACN]; B %: 19%-49%,8 min). Afford 10 mg crude product. The crude product was purified by prep-HPLC (column: Waters Xbridge BEH C18 100*25 mm*5 um; mobile phase:[water(10 mM NH₄HCO₃)-ACN]; B %: 15%-55%,10 min). Compound 2-[[6-[(4,4-difluorocyclohexyl)amino]-3-morpholinosulfonyl-4-quinolyl]amino]benzoic acid (2.56 mg, 4.68 umol, 11.53% yield, 100% purity) was obtained as a yellow solid. ¹H NMR (400 MHz, DMSO-d₆) δ=10.39 (br s, 1H), 8.71 (s, 1H), 7.97 (br d, J=7.8 Hz, 1H), 7.81 (d, J=9.0 Hz, 1H), 7.38-7.20 (m, 2H), 6.94 (br t, J=7.5 Hz, 1H), 6.40 (br dd, J=7.6, 17.3 Hz, 2H), 6.23 (s, 1H), 3.49-3.42 (m, 2H), 3.31 (br d, J=7.9 Hz, 2H), 3.06-2.89 (m, 5H), 2.08-1.96 (m. 1H), 1.90-1.72 (m, 3H), 1.59-1.24 (m, 3H), 1.15-0.97 (m, 1H). MS (M+H)⁺=547.2.

### Example 57A—Synthesis of Compound 239A

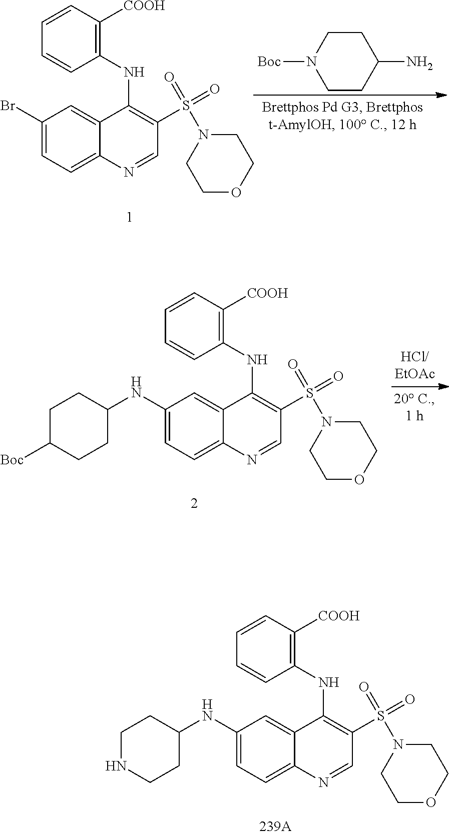 [SMILES: O=C(O)c1ccccc1Nc1c(S(=O)(=O)N2CCOCC2)cnc2ccc(Br)cc12 ; CC(C)(C)OC(=O)N1CCC(N)CC1 ; CC(C)(C)OC(=O)C1CCC(Nc2ccc3ncc(S(=O)(=O)N4CCOCC4)c(Nc4ccccc4C(=O)O)c3c2)CC1 ; O=C(O)c1ccccc1Nc1c(S(=O)(=O)N2CCOCC2)cnc2ccc(NC3CCNCC3)cc12]

Step 1. 2-[[6-[(1-tert-butoxycarbonyl-4-piperidyl)amino]-3-morpholinosulfonyl-4-quinolyl]amino]benzoic acid (2): To a stirred solution of 2-[(6-bromo-3-morpholinosulfonyl-4-quinolyl)amino]benzoic acid (30 mg, 60.93 umol, 1 eq) in 2-methylbutan-2-ol (1 mL) was added tert-butyl 4-aminopiperidine-1-carboxylate (12.20 mg, 60.93 umol, 1 eq), BRETTPHOS (3.27 mg, 6.09 umol, 0.1 eq), BrettPhos Pd G3 (5.52 mg, 6.09 umol, 0.1 eq) and t-BuONa (17.57 mg, 182.80 umol, 3 eq) the mixture was bubbled with N₂ for 1 minute, and stirred at 100° C. for 12 h. LCMS showed the starting material was consumed completely and desired MS was detected. The reaction mixture was filtered, and filtrate was purified by prep-HPLC (column: Welch Xtimate C18 100*25 mm*3 um; mobile phase: [water(0.04% HCl)-ACN]; B %: 10%-30%,8 min). Compound 2-[[6-[(1-tert-butoxycarbonyl-4-piperidyl)amino]-3-morpholinosulfonyl-4-quinolyl]amino]benzoic acid (15 mg, 23.14 umol, 37.98% yield, HCl) was obtained as a yellow solid. MS (M+H)⁺=612.5.

Step 2. Synthesis of 2-[[3-morpholinosulfonyl-6-(4-piperidylamino)-4-quinolyl]amino]benzoic acid (239A): A solution of 2-[[6-[(1-tert-butoxycarbonyl-4-piperidyl)amino]-3-morpholinosulfonyl-4-quinolyl]amino]benzoic acid (15 mg, 24.52 umol, 1 eq) in HCl/EtOAc (4M, 1 mL, 163.12 eq) was stirred at 20° C. for 1 h. LCMS showed the starting material was consumed completely and desired MS was detected. The reaction mixture was concentrate in vacuum. The residue was purified by prep-HPLC (column: Phenomenex luna C18 80*40 mm*3 um; mobile phase: [water(0.04% HCl)-ACN]; B %: 3%-38%,7 min). Compound 2-[[3-morpholinosulfonyl-6-(4-piperidylamino)-4-quinolyl]amino]benzoic acid (2.21 mg, 4.00 umol, 16.31% yield, 99.19% purity, HCl) was obtained as a yellow solid. ¹H NMR (400 MHz, DMSO-d₆+D₂O) δ=8.79 (s, 1H), 7.99 (dd, J=1.4, 7.9 Hz, 1H), 7.87 (d, J=9.2 Hz, 1H), 7.43-7.33 (m, 2H), 7.10 (t, J=7.6 Hz, 1H), 6.63 (d, J=8.1 Hz, 1H), 6.17 (d, J=2.2 Hz, 1H), 3.53-3.43 (m, 2H), 3.39-3.30 (m, 2H), 3.27-3.18 (m, 1H), 3.14-2.94 (m, 6H), 2.92-2.77 (m, 1H), 2.45-2.38 (m, 1H), 1.83 (br dd, J=1.2, 12.3 Hz, 1H), 1.64-1.53 (m, 1H), 1.45 (br d, J=11.5 Hz, 1H), 1.21-1.11 (m, 1H). MS (M+H)⁺=512.2.

### Example 58A—Synthesis of Compound 240A

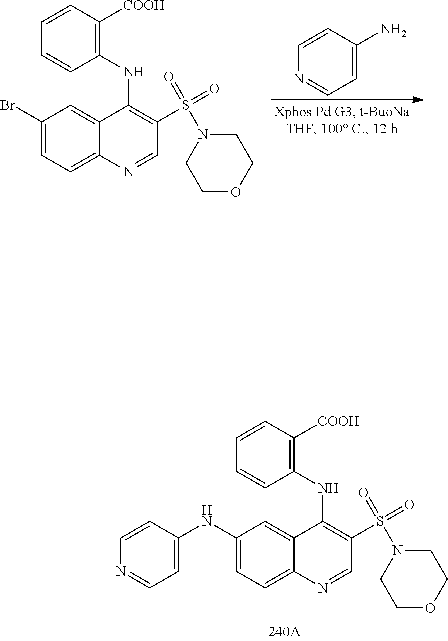 [SMILES: O=C(O)c1ccccc1Nc1c(S(=O)(=O)N2CCOCC2)cnc2ccc(Br)cc12 ; Nc1ccncc1 ; O=C(O)c1ccccc1Nc1c(S(=O)(=O)N2CCOCC2)cnc2ccc(Nc3ccncc3)cc12]

To a solution of methyl 2-[(6-bromo-3-morpholinosulfonyl-4-quinolyl)amino]benzoate (20 mg, 39.50 umol, 1 eq) in DMF (0.5 mL) was added XPhos Pd G3 (3.34 mg, 3.95 umol, 0.1 eq), NaOtBu (7.59 mg, 78.99 umol, 2 eq) and pyridin-4-amine (15.00 mg, 159.38 umol, 26.79 uL, 4.04 eq), the mixture was pueged with N₂, the reaction mixture was stirred at 100° C. for 12 h under N2. LCMS showed starting material was consumed completely and the MS of desired product was detected. The reaction was concentrated in vacuum. The crude product was purified by prep-HPLC (column: Welch Xtimate C18 100*25 mm*3 um; mobile phase: [water(0.04% HCl)-ACN]; B %: 5%-25%,8 min). Compound 2-[[3-morpholinosulfonyl-6-(4-pyridylamino)-4-quinolyl]amino]benzoic acid (2.28 mg, 4.51 umol, 11.42% yield, 100% purity) was obtained as a yellow oil. ¹H NMR (400 MHz, DMSO-d₆) ppm 10.87 (s, 1H), 10.36 (br s, 1H), 9.08 (s, 1H), 8.12-8.30 (m, 3H), 7.99 (dd, J=7.94, 1.31 Hz, 1H), 7.82 (dd, J=9.07, 2.31 Hz, 1H), 7.57 (d, J=2.25 Hz, 1H), 7.41-7.52 (m, 1H), 7.12 (t, J=7.63 Hz, 1H), 6.97 (d, J=7.13 Hz, 2H), 6.77 (d, J=8.25 Hz, 1H), 3.48 (br d, J=2.38 Hz, 2H), 3.27-3.41 (m, 2H), 3.01-3.10 (m, 4H). MS (M+H)⁺=506.1.

### Example 59A—Synthesis of Compound 241A

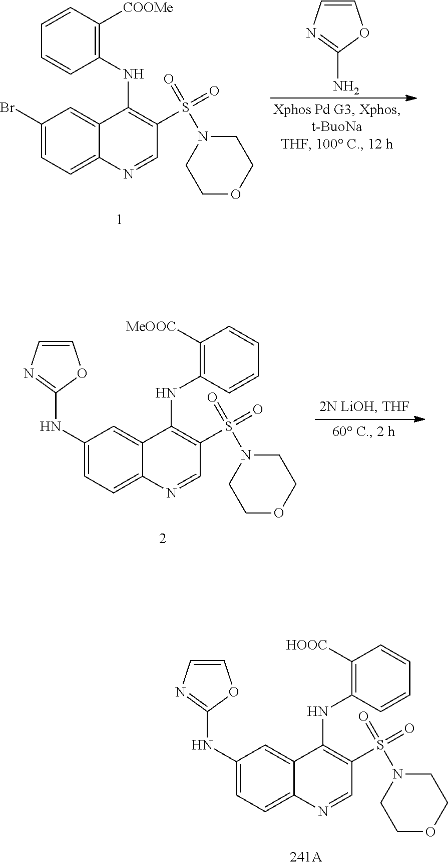 [SMILES: COC(=O)c1ccccc1Nc1c(S(=O)(=O)N2CCOCC2)cnc2ccc(Br)cc12 ; Nc1ncco1 ; COC(=O)c1ccccc1Nc1c(S(=O)(=O)N2CCOCC2)cnc2ccc(Nc3ncco3)cc12 ; O=C(O)c1ccccc1Nc1c(S(=O)(=O)N2CCOCC2)cnc2ccc(Nc3ncco3)cc12]

Step 1. Synthesis of methyl 2-[[3-morpholinosulfonyl-6-(oxazol-2-ylamino)-4-quinolyl]amino]benzoate (2): To a stirred solution of methyl 2-[(6-bromo-3-morpholinosulfonyl-4-quinolyl)amino]benzoate (30 mg, 59.25 umol, 1 eq) in THF (2 mL) was added oxazol-2-amine (4.98 mg, 59.25 umol, 1 eq). XPhos Pd G3 (5.01 mg, 5.92 umol, 0.1 eq), XPhos (2.82 mg, 5.92 umol, 0.1 eq) and t-BuONa (17.08 mg, 177.74 umol, 3 eq) the mixture was bubbled with N₂ for 1 minute, and stirred at 100° C. for 12 h. LCMS showed the starting material ws consumed completely and desired MS was detected. The reaction mixture was filtered, and filtrate was purified by prep-HPLC (column: Phenomenex Gemini NX-C18(75*30 mm*3 um); mobile phase: [water(0.04% HCl)-ACN]; B %: 10%-40%,8 min). Compound methyl 2-[[3-morpholinosulfonyl-6-(oxazol-2-ylamino)-4-quinolyl]amino]benzoate (10 mg, 19.63 umol, 33.13% yield) was obtained as a yellow solid. MS (M+H)⁺=510.4.

Step 2. Synthesis of 2-[[3-morpholinosulfonyl-6-(oxazol-2-ylamino)-4-quinolyl]amino]benzoic acid (241A): A solution of methyl 2-[[3-morpholinosulfonyl-6-(oxazol-2-ylamino)-4-quinolyl]amino]benzoate (10 mg, 19.63 umol, 1 eq) and LIOH (2 M, 19.63 uL, 2 eq) in THF (0.5 mL) was stirred at 60° C. for 2 h. LCMS showed the starting material was consumed completely and desired MS was detected. The reaction mixture was concentrate in vacuum. The residue was purified by prep-HPLC (column: Phenomenex Gemini NX-C18(75*30 mm*3 um); mobile phase: [water(0.04% HCl)-ACN]; B %: 5%-35%,8 min). Compound 2-[[3-morpholinosulfonyl-6-(oxazol-2-ylamino)-4-quinolyl]amino]benzoic acid (2.03 mg, 3.69 umol, 18.79% yield, 96.63% purity, HCl) was obtained as a yellow solid. ¹H NMR (400 MHz, DMSO-d₆) δ=10.72 (s, 1H), 10.55 (s, 1H), 9.01 (s, 1H), 8.22 (d, J=2.0 Hz, 1H), 8.15-8.10 (m, 1H), 8.08-8.00 (m, 2H), 7.56 (s, 1H), 7.34 (t, J=7.3 Hz, 1H), 7.12 (t, J=7.5 Hz, 1H), 6.82 (br d, J=8.4 Hz, 1H), 6.74 (s, 1H), 3.59-3.52 (m, 2H), 3.48-3.39 (m, 2H), 3.18-3.04 (m, 4H). MS (M+H)⁺=496.1.

### Example 60A—Synthesis of Compound 247A

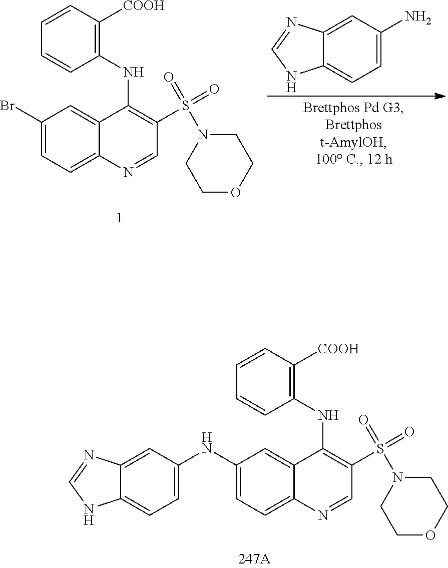 [SMILES: O=C(O)c1ccccc1Nc1c(S(=O)(=O)N2CCOCC2)cnc2ccc(Br)cc12 ; Nc1ccc2[nH]cnc2c1 ; O=C(O)c1ccccc1Nc1c(S(=O)(=O)N2CCOCC2)cnc2ccc(Nc3ccc4[nH]cnc4c3)cc12]

Synthesis of 2-[[6-(1H-benzimidazol-5-ylamino)-3-morpholinosulfonyl-4-quinolyl]amino]benzoic acid (247A): To a stirred solution of 2-[(6-bromo-3-morpholinosulfonyl-4-quinolyl)amino]benzoic acid (40 mg, 81.24 umol, 1 eq) in 2-METHYL-2-BUTANOL (1 mL) was added 1H-benzimidazol-5-amine (10.82 mg, 81.24 umol, 1 eq), BrettPhos Pd G3 (7.36 mg, 8.12 umol, 0.1 eq), t-BuONa (23.42 mg, 243.73 umol, 3 eq) and BRETTPHOS (4.36 mg, 8.12 umol, 0.1 eq) the mixture was bubbled with N₂ for 1 minute, and stirred at 100° C. for 12 h. LCMS showed the starting material was consumed completely and desired MS was detected. The reaction mixture was filtered, and filtrate was purified by prep-HPLC (column: Phenomenex Gemini-NX C18 75*30 mm*3 um; mobile phase: [water(0.04% HCl)-ACN]; B %: 5%-30%,8 min). Compound 2-[[6-(1H-benzimidazol-5-ylamino)-3-morpholinosulfonyl-4-quinolyl]amino]benzoic acid (12.08 mg, 20.72 umol, 25.510% yield, 99.68% purity, HCl) was obtained as a yellow solid. ¹H NMR (400 MHz, DMSO-d₆) δ=10.47 (s, 1H), 9.50 (s, 1H), 9.37 (br s, 1H), 8.91 (s, 1H), 8.15 (d, J=9.3 Hz, 1H), 7.93-7.84 (m, 1H), 7.80-7.67 (m, 1H), 7.49-7.41 (m, 2H), 7.36 (d, J=1.6 Hz, 1H), 7.08 (t, J=7.6 Hz, 1H), 7.02-6.92 (m, 2H), 6.91-6.82 (m, 1H), 3.56-3.47 (m, 2H), 3.43-3.35 (m, 2H), 3.18-2.99 (m, 4H). MS (M+H)⁺=545.2.

### Example 61—Synthesis of Compound 248A

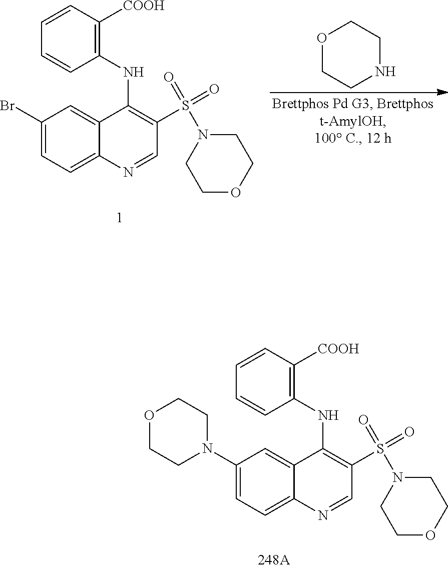 [SMILES: O=C(O)c1ccccc1Nc1c(S(=O)(=O)N2CCOCC2)cnc2ccc(Br)cc12 ; C1COCCN1 ; O=C(O)c1ccccc1Nc1c(S(=O)(=O)N2CCOCC2)cnc2ccc(N3CCOCC3)cc12]

To a stirred solution of 2-[(6-bromo-3-morpholinosulfonyl-4-quinolyl)amino]benzoic acid (40 mg, 81.24 umol, 1 eq) in 2-METHYL-2-BUTANOL (1 mL) was added morpholine (7.08 mg, 81.24 umol, 7.15 uL, 1 eq), BrettPhos Pd G3 (7.36 mg, 8.12 umol, 0.1 eq), BRETTPHOS (4.36 mg, 8.12 umol, 0.1 eq) and t-BuONa (23.42 mg, 243.72 umol, 3 eq) the mixture was bubbled with N₂ for 1 minute, and stirred at 100° C. for 12 h. LCMS showed the starting material was consumed completely and desired MS was detected. The reaction mixture was filtered, and filtrate was purified by prep-HPLC (column: Welch Xtimate C18 100*25 mm*3 um; mobile phase: [water(0.04% HCl)-ACN]; B %: 10%-40%,8 min). Compound 2-[(6-morpholino-3-morpholinosulfonyl-4-quinolyl)amino]benzoic acid (12.86 mg, 24.04 umol, 29.59% yield, 100% purity, HCl) was obtained as a yellow solid. ¹H NMR (400 MHz, DMSO-d₆) δ=10.72-10.60 (m. 1H), 8.95 (s, 1H), 8.12-7.99 (m, 2H), 7.87 (td, J=3.0, 6.1 Hz, 1H), 7.49-7.41 (m, 1H), 7.24-7.12 (m, 1H), 6.96-6.80 (m, 1H), 6.63 (br s, 1H), 3.66-3.57 (m, 4H), 3.54 (br d, J=12.5 Hz, 2H), 3.49-3.39 (m, 2H), 3.11 (br s, 4H), 3.02 (br dd, J=5.0, 12.3 Hz, 2H), 2.80-2.72 (m, 2H). MS (M+H)⁺=499.2.

### Example 62—Synthesis of Compound 249A

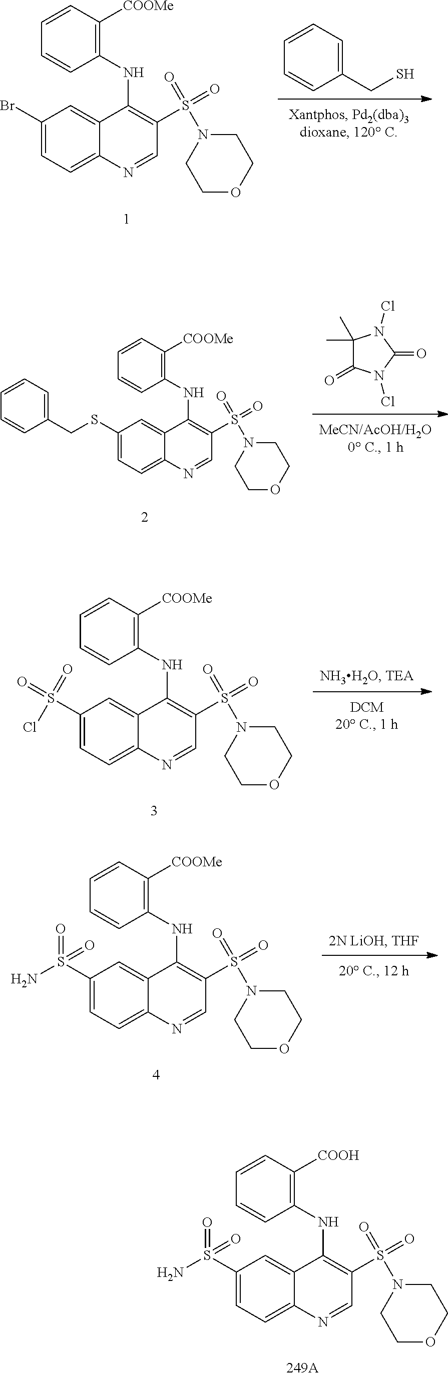

Step 1. Synthesis of methyl 2-[(6-benzylsulfanyl-3-morpholinosulfonyl-4-quinolyl)amino]benzoate (2): To a stirred solution of methyl 2-[(6-bromo-3-morpholinosulfonyl-4-quinolyl)amino]benzoate (200 mg, 394.97 umol, 1 eq) in dioxane (4 mL) was added phenylmethanethiol (53.96 mg, 434.47 umol, 50.91 uL, 1.1 eq), Xantphos (22.85 mg, 39.50 umol, 0.1 eq), Pd2(dba)₃ (36.17 mg, 39.50 umol, 0.1 eq) and DIPEA (102.09 mg, 789.94 umol, 137.59 uL, 2 eq), the mixture was bubbled with N₂ for 1 minute, and stirred at 100° C. for 12 h. LCMS showed the starting material was consumed completely and desired MS was detected. The reaction mixture was poured into water (10 mL). The aqueous phase was extracted with ethyl acetate (20 mL*2). The combined organic phase was dried with anhydrous Na₂SO₄, filtered and concentrated in vacuum. The crude product was purified by flash column (ISCO 10 g silica, 0-10% ethyl acetate in petroleum ether, gradient over 20 min). Based on TLC (Petroleum ether:Ethyl acetate=1/1, R_{f}=0.45). Compound methyl 2-[(6-benzylsulfanyl-3-morpholinosulfonyl-4-quinolyl)amino]benzoate (200 mg, 363.86 umol, 92.12% yield) was obtained as a yellow oil. MS (M+H)⁺=550.3. ¹H NMR (400 MHz, CHLOROFORM-d) δ=10.42 (s, 1H), 9.12 (s, 1H), 8.09 (dd, J=1.6, 8.0 Hz, 1H), 8.00 (d, J=8.9 Hz, 1H), 7.62 (dd, J=2.1, 8.9 Hz, 1H), 7.42 (d, J=2.0 Hz, 1H), 7.32-7.28 (m, 1H), 7.25-7.19 (m, 3H), 7.13-7.09 (m, 2H), 7.06-6.95 (m, 1H), 6.50 (d, J=8.1 Hz, 1H), 4.03 (s, 3H), 3.81 (d, J=1.8 Hz, 2H), 3.66-3.58 (m, 2H), 3.56-3.47 (m, 2H), 3.20-3.04 (m, 4H).

### Step 2. Synthesis of methyl 2-[(6-chlorosulfonyl-3-morpholinosulfonyl-4-quinolyl)amino]benzoate (3)

A solution of methyl 2-[(6-benzylsulfanyl-3-morpholinosulfonyl-4-quinolyl)amino]benzoate (100 mg, 181.93 umol, 1 eq) in MeCN (4 mL), AcOH (1.6 mL) and H₂O (1.6 mL) was stirred at 20° C. for 0.5 h. 1,3-dichloro-5,5-dimethyl-imidazolidine-2,4-dione (71.69 mg, 363.86 umol, 2 eq) was added at 0° C., and the mixture was stirred at 0° C. for 0.5 h. LCMS showed the starting material was consumed completely and desired MS was detected. The reaction mixture was poured into water (10 mL). The aqueous phase was extracted with dichloromethane (20 mL*2). The combined organic phase was dried with anhydrous Na₂SO₄, filtered and concentrated in vacuum. Compound methyl 2-[(6-chlorosulfonyl-3-morpholinosulfonyl-4-quinolyl)amino]benzoate (100 mg, crude) was obtained as yellow oil.

Step 3. Synthesis of methyl 2-[(3-morpholinosulfonyl-6-sulfamoyl-4-quinolyl)amino]benzoate (4): To a stirred solution of methyl 2-[(6-chlorosulfonyl-3-morpholinosulfonyl-4-quinolyl)amino]benzoate (100 mg, 190.12 umol, 1 eq) in DCM (2 mL) was added NH₃·H₂O (99.94 mg, 570.36 umol, 109.83 uL, 20% purity, 3 eq) TEA (57.71 mg, 570.36 umol, 79.39 uL, 3 eq) the mixture was stirred at 20° C. for 0.5 h. LCMS showed the starting material was consumed completely and desired MS was detected. The reaction mixture was poured into water (10 mL). The aqueous phase was extracted with dichloromethane (20 mL*2). The combined organic phase was dried with anhydrous Na₂SO₄, filtered and concentrated in vacuum. Compound methyl 2-[(3-morpholinosulfonyl-6-sulfamoyl-4-quinolyl)amino]benzoate (100 mg, crude) was obtained as yellow oil.

Step 4. Synthesis of 2-[(3-morpholinosulfonyl-6-sulfamoyl-4-quinolyl)amino]benzoic acid (249A): A solution of methyl 2-[(3-morpholinosulfonyl-6-sulfamoyl-4-quinolyl)amino]benzoate (10 mg, 19.74 umol, 1 eq) and LiOH (2 M, 19.74 uL, 2 eq) in THF (1 mL) was stirred at 20° C. for 2 h. LCMS showed the starting material was consumed completely and desired MS was detected. The reaction mixture was adjusted pH˜4 by adding 2N HCl. The mixture was concentrate in vacuum. The residue was purified by prep-HPLC (column: Phenomenex luna C18 80*40 mm*3 um; mobile phase: [water(0.04% HCl)-ACN]; B %: 20%-50%,7 min). Compound 2-[(3-morpholinosulfonyl-6-sulfamoyl-4-quinolyl)amino]benzoic acid (2.37 mg, 4.38 umol, 22.18% yield, 97.71% purity, HCl) was obtained as a yellow solid. ¹H NMR (400 MHz, DMSO-d₆) δ=10.47 (br s, 1H), 9.16 (s, 1H), 8.31-8.23 (m, 1H), 8.21-8.12 (m, 2H), 8.00 (dd, J=1.5, 7.9 Hz, 1H), 7.45 (s, 2H), 7.32 (t, J=7.8 Hz, 1H), 7.07 (t, J=7.6 Hz, 1H), 6.73 (br d, J=8.3 Hz, 1H), 3.52-3.48 (m, 2H), 3.41-3.33 (m, 2H), 3.14-2.98 (m, 4H). MS (M+H)⁺=493.1.

### Example 63—Synthesis of Compound 250A

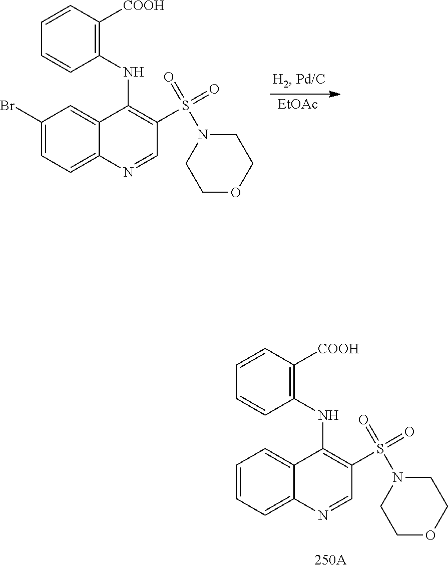 [SMILES: "EtOAc" ; O=C(O)c1ccccc1Nc1c(S(=O)(=O)N2CCOCC2)cnc2ccc(Br)cc12 ; O=C(O)c1ccccc1Nc1c(S(=O)(=O)N2CCOCC2)cnc2ccccc12]

To a solution of 2-[(6-bromo-3-morpholinosulfonyl-4-quinolyl)amino]benzoic acid (100 mg, 203.11 umol, 1 eq) in EtOAc (1 mL) was added Pd/C (24.05 mg, 20.31 umol, 10% purity, 0.1 eq), the mixture was purged with H₂ for 3 times, the reaction mixture was stirred at 100° C. for 12 h under H₂. LCMS showed the starting material was consumed completely and the MS of desired product was detected. The reaction mixture was concentrated in vacuum. The crude product was purified by prep-HPLC (column: Phenomenex Luna C18 150*30 mm*5 um; mobile phase: [water (0.04% HCl)-ACN]; B %: 10%-45%,8 min). Compound 2-[(3-morpholinosulfonyl-4-quinolyl)amino]benzoic acid (36.12 mg, 79.84 umol, 39.31% yield, 99.45% purity, HCl) was obtained as a yellow solid. ¹H NMR (400 MHz, DMSO-d₆) δ ppm 10.53 (br s, 1H), 9.07-9.18 (m, 1H), 8.14 (d, J=8.38 Hz, 1H), 8.01 (dd, J=7.94, 1.56 Hz, 1H), 7.92 (td, J=7.69, 1.25 Hz, 1H), 7.66 (d, J=8.00 Hz, 1H), 7.46-7.55 (m, 1H), 7.29-7.39 (m, 1H), 7.05-7.15 (m, 1H), 6.75 (br d, J=8.25 Hz, 1H), 3.47-3.55 (m, 2H), 3.34-3.43 (m, 2H), 2.99-3.15 (m, 4H). MS (M+H)⁺=414.1.

### Example 64—Synthesis of 261

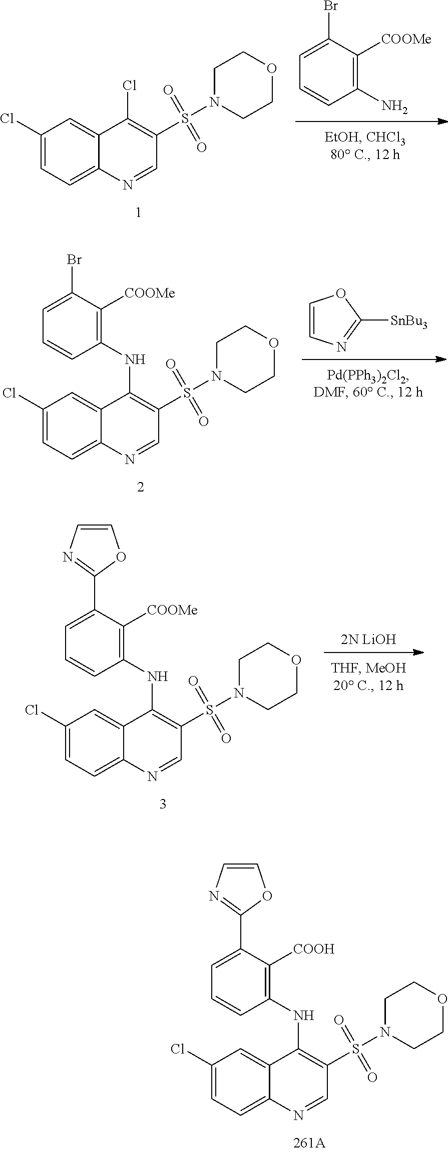

Step 1. Synthesis of methyl 2-bromo-6-[(6-chloro-3-morpholinosulfonyl-4-quinolyl)amino]benzoate (2): A solution of methyl 2-amino-6-bromo-benzoate (66.26 mg, 288.00 umol, 1 eq) and 4-[(4,6-dichloro-3-quinolyl)sulfonyl]morpholine (100 mg, 288.00 umol, 1 eq) in ACN (2 mL) was stirred at 80° C. for 12 h. LCMS showed the starting material was consumed completely and desired MS was detected. The reaction mixture was filtered, and filter cake was concentrate in vacuum. Compound methyl 2-bromo-6-[(6-chloro-3-morpholinosulfonyl-4-quinolyl)amino]benzoate (150 mg, 277.36 umol, 96.30% yield) was obtained as a yellow solid. MS (M+H)⁺=542.1.

Step 2. Synthesis of methyl 2-[(6-chloro-3-morpholinosulfonyl-4-quinolyl)amino]-6-oxazol-2-yl-benzoate (3): To a stirred solution of methyl 2-bromo-6-[(6-chloro-3-morpholinosulfonyl-4-quinolyl)amino]benzoate (150 mg, 277.36 umol, 1 eq) in DMF (1 mL) was added tributyl(oxazol-2-yl)stannane (794.59 mg, 2.22 mmol, 8 eq) and Pd(PPh₃)₂Cl₂ (19.47 mg, 27.74 umol, 0.1 eq) the mixture was bubbled with N₂ for 1 minute, and stirred at 60° C. for 12 h. LCMS showed the desired MS was detected. The reaction mixture was filtered, and filtrate was purified directly. The filtrate was purified by prep-HPLC (column: Waters Xbridge BEH C18 100*30 mm*10 um; mobile phase: [water(10 mM NH₄HCO₃)-ACN]; B %: 30%-50%,8 min). Compound methyl 2-[(6-chloro-3-morpholinosulfonyl-4-quinolyl)amino]-6-oxazol-2-yl-benzoate (20 mg, 37.81 umol, 13.63% yield) was obtained as white solid. MS (M+H)⁺=529.3.

Step 3. Synthesis of 2-[(6-chloro-3-morpholinosulfonyl-4-quinolyl)amino]-6-oxazol-2-yl-benzoic acid (261A): To a stirred solution of methyl 2-[(6-chloro-3-morpholinosulfonyl-4-quinolyl)amino]-6-oxazol-2-yl-benzoate (18 mg, 34.03 umol, 1 eq) in THF (0.5 mL) and MEOH (0.5 mL) was added LiOH·H₂O (2 M, 68.06 uL, 4 eq) at 20° C., and the mixture was stirred at 20° C. for 12 h. LCMS showed the starting material was consumed completely and desired MS was detected. The reaction mixture was concentrate in vacuum. The residue was purified by prep-HPLC (column: Phenomenex Luna 80*30 mm*3 um; mobile phase: [water(0.04% HCl)-ACN]; B %: 20%-50%,8 min). Afford 6 mg crude product. The crude product was purified by prep-HPLC (column: Phenomenex Luna 80*30 mm*3 um; mobile phase: [water(TFA)-ACN]; B %: 30%-65%,8 min). Compound 2-[(6-chloro-3-morpholinosulfonyl-4-quinolyl)amino]-6-oxazol-2-yl-benzoic acid (1.0 mg, 1.59 umol, 4.67% yield, 100% purity, TFA) was obtained as a yellow solid. ¹H NMR (400 MHz, DMSO-d₆) δ=9.06-8.94 (m, 2H), 8.31 (s, 1H), 8.16-8.04 (m, 1H), 7.92-7.82 (m, 1H), 7.68 (br d, J=7.4 Hz, 1H), 7.52-7.38 (m, 3H), 7.03 (br d, J=8.0 Hz, 1H), 3.61-3.55 (m, 4H), 3.15-3.07 (m, 4H). MS (M+H)⁺=515.0.

### Example 65—Synthesis of 262A

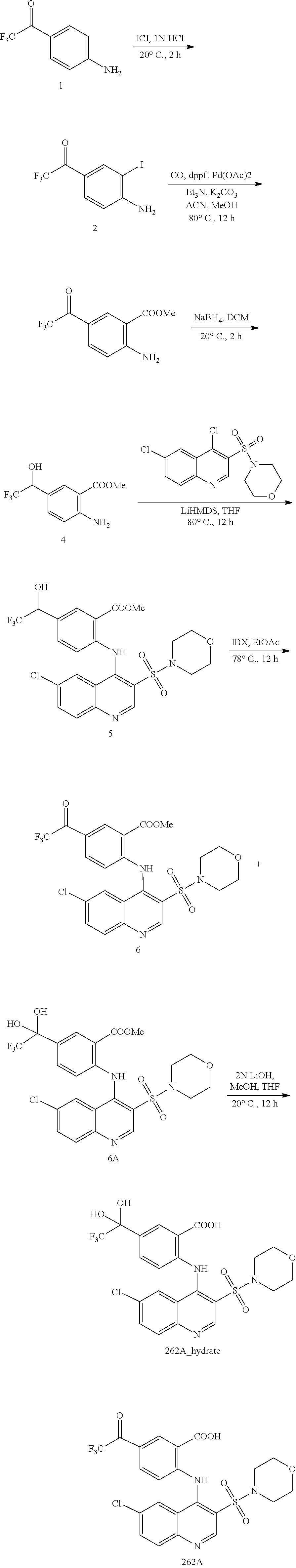

Synthesis 1. Synthesis of 1-(4-amino-3-iodo-phenyl)-2,2,2-trifluoro-ethanone (2): To a stirred solution of 1-(4-aminophenyl)-2,2,2-trifluoro-ethanone (1 g, 5.29 mmol, 1 eq) in HCl (1 M, 53.71 mL, 10.16 eq) was added IODINEMONOCHLORIDE (772.59 mg, 4.76 mmol, 242.95 uL, 0.9 eq) at 20° C., and the mixture was stirred at 20° C. for 2 h. TLC (Petroleum ether:ethyl acetate=5:1,R_{f}=0.46) showed a little starting material was remained and one main spot was formed. The reaction mixture was adjusted pH-8 by adding sat. NaHCO₃. The mixture was extracted with ethyl acetate (100 mL*3). The combined organic layer was dried with Na₂SO₄, filtered and filtrate was concentrated in vacuum to give a crude product. The residue was purified by flash column (ISCO 40 g silica, 5-15% ethyl acetate in petroleum ether, gradient over 20 min). Compound 1-(4-amino-3-iodo-phenyl)-2,2,2-trifluoro-ethanone (1.2 g, 3.81 mmol, 72.04% yield) was obtained as a white solid. ¹H NMR (400 MHz, CHLOROFORM-d) δ ppm 8.30 (d, J=1.00 Hz, 1H), 7.73-7.84 (m, 1H), 6.67 (d, J=8.63 Hz, 1H), 4.87 (br s, 2H).

Step 2. Synthesis of methyl 2-amino-5-(2,2,2-trifluoroacetyl)benzoate (3): To a stirred solution of 1-(4-amino-3-iodo-phenyl)-2,2,2-trifluoro-ethanone (1 g, 3.17 mmol, 1 eq) in ACN (7 mL) and MeOH (15 mL) was added DPPF (175.98 mg, 317.43 umol, 0.1 eq), Pd(OAc)₂ (71.27 mg, 317.43 umol, 0.1 eq), K₂CO₃ (1.32 g, 9.52 mmol, 3 eq) and TEA (321.20 mg, 3.17 mmol, 441.82 uL, 1 eq), the mixture was purged with CO three times, and stirred at 80° C. for 4 h. TLC (Petroleum ether/Ethyl acetate=3:1, R_{f}=0.40) showed starting material was consumed completely and new spot was formed. The reaction mixture was poured into water (10 mL). The aqueous phase was extracted with ethyl acetate (30 mL*3). The combined organic phase was dried with anhydrous Na₂SO₄, filtered and concentrated in vacuum. The residue was purified by flash column (ISCO 10 g silica, 12-16% Ethyl acetate in Petroleum ether, gradient over 15 min). Compound methyl 2-amino-5-(2,2,2-trifluoroacetyl)benzoate (300 mg, 1.21 mmol, 38.24% yield) was obtained as a yellow solid. ¹H NMR (400 MHz, CHLOROFORM-d) δ=8.67 (s, 1H), 7.96 (dd, J=0.7, 8.8 Hz, 1H), 6.72 (d, J=8.9 Hz, 1H), 3.93 (s, 3H).

Step 3. Synthesis of methyl 2-amino-5-(2,2,2-trifluoro-1-hydroxy-ethyl)benzoate (4): To a stirred solution of methyl 2-amino-5-(2,2,2-trifluoroacetyl)benzoate (300 mg, 1.21 mmol, 1 eq) in DCM (5 mL) was added NaBH₄ (91.84 mg, 2.43 mmol, 2 eq) at 20° C., the mixture was stirred at 20° C. for 4 h. TLC (Petroleum ether/Ethyl acetate=1:1, R_{f}=0.61) showed starting material was consumed completely and new spot was formed. The reaction mixture was poured into sat NH₄Cl (10 mL) The aqueous phase was extracted with dichloromethane (20 mL*2). The combined organic phase was dried with anhydrous Na₂SO₄, filtered and concentrated in vacuum. The residue was purified by flash column (ISCO 10 g silica, 50-60% Ethyl acetate in Petroleum ether, gradient over 15 min). Compound methyl 2-amino-5-(2,2,2-trifluoro-1-hydroxy-ethyl)benzoate (210 mg, 842.74 umol, 69.43% yield) was obtained as a white solid. MS (M+H)⁺=250.2.

Step 4. Synthesis of methyl 2-[(6-chloro-3-morpholinosulfonyl-4-quinolyl)amino]-5-(2,2,2-trifluoro-1-hydroxy-ethyl)benzoate (5): To a solution of 4-[(4,6-dichloro-3-quinolyl)sulfonyl]morpholine (278.68 mg, 802.61 umol, 1 eq) in THF (3 mL) was added LiHMDS (1 M, 2.41 mL, 3 eq) dropwise. The mixture was purged with N2, the mixture was stirred at 20° C. for 30 minutes then the mixture was added methyl 2-amino-5-(2,2,2-trifluoro-1-hydroxy-ethyl)benzoate (200.00 mg, 802.61 umol, 1 eq), the solution was purged with N2, the reaction was stirred at 80° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 30%-60%,8 min). Compound methyl 2-[(6-chloro-3-morpholinosulfonyl-4-quinolyl)amino]-5-(2,2,2-trifluoro-1-hydroxy-ethyl)benzoate (25 mg, 44.65 umol, 5.56% yield) was obtained as a yellow solid. MS (M+H)⁺=560.2.

Step 5. Synthesis of methyl 2-[(6-chloro-3-morpholinosulfonyl-4-quinolyl)amino]-5-(2,2,2-trifluoroacetyl)benzoate & methyl 2-[(6-chloro-3-morpholinosulfonyl-4-quinolyl)amino]-5-(2,2,2-trifluoro-1,1-dihydroxy-ethyl)benzoate (6&6A): To a solution of methyl 2-[(6-chloro-3-morpholinosulfonyl-4-quinolyl)amino]-5-(2,2,2-trifluoro-1-hydroxy-ethyl)benzoate (15 mg, 26.79 umol, 1 eq) in EtOAc (0.5 mL) was added IBX (30.00 mg, 107.15 umol, 4 eq), the mixture was purged with N₂, the reaction was stirred at 78° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)- Compound methyl 2-[(6-chloro-3-morpholinosulfonyl-4-quinolyl)amino]-5-(2,2,2-trifluoroacetyl)benzoate (5 mg, 8.41 umol, 31.40% yield, HCl) was obtained as a yellow solid.methyl 2-[(6-chloro-3-morpholinosulfonyl-4-quinolyl)amino]-5-(2,2,2-trifluoro-1,1-dihydroxy-ethyl)benzoate (15 mg, 24.49 umol, 91.43% yield, HCl) was obtained as a yellow solid. MS (M+H)⁺=558.1; 576.1.

Step 6. Synthesis of 2-[(6-chloro-3-morpholinosulfonyl-4-quinolyl)amino]-5-(2,2,2-trifluoro-1,1-dihydroxy-ethyl)benzoic acid & 2-[(6-chloro-3-morpholinosulfonyl-4-quinolyl)amino]-5-(2,2,2-trifluoroacetyl)benzoic acid (262A&262A_hydrate): To a stirred solution of methyl 2-[(6-chloro-3-morpholinosulfonyl-4-quinolyl)amino]-5-(2,2,2-trifluoro-1,1-dihydroxyethyl)benzoate (15 mg, 26.04 umol, 1 eq) in THF (0.4 mL) and MeOH (0.4 mL) was added LiOH·H₂O (2 M, 26.04 uL, 2 eq) at 25° C., then the mixture was stirred at 25° C. for 12 h. LCMS showed the starting material was consumed completely and desired Ms was detected. The reaction mixture was adjusted pH˜4 by adding 2N HCl. The mixture was purified by prep-HPLC (column: Phenomenex luna C18 80*40 mm*3 um; mobile phase: [water(HCl)-ACN]; B %: 40%-60%,7 min). A mixture of 2-[(6-chloro-3-morpholinosulfonyl-4-quinolyl)amino]-5-(2,2,2-trifluoro-1,1-dihydroxy-ethyl)benzoic acid (2.5 mg, 4.18 umol, 16.04% yield, 100% purity, HCl) and 2-[(6-chloro-3-morpholinosulfonyl-4-quinolyl)amino]-5-(2,2,2-trifluoroacetyl)benzoic acid (HCl) (ratio=3/2) was obtained as a yellow solid. ¹H NMR (400 MHz, DMSO-d₆) δ=10.44 (br s, 1H), 9.12 (s, 1H), 8.24 (d, J=2.1 Hz, 1H), 8.16 (d, J=9.0 Hz, 1H), 7.92 (dd, J=2.3, 8.9 Hz, 1H), 7.62 (br d, J=2.3 Hz, 2H), 7.48 (dd, J=2.2, 8.7 Hz, 1H), 6.64 (d, J=8.7 Hz, 1H), 3.33-3.27 (m, 4H), 3.09-2.96 (m, 4H). MS (M+H)⁺=543.9; 561.9.

### Example 66—Synthesis of Compound 263A

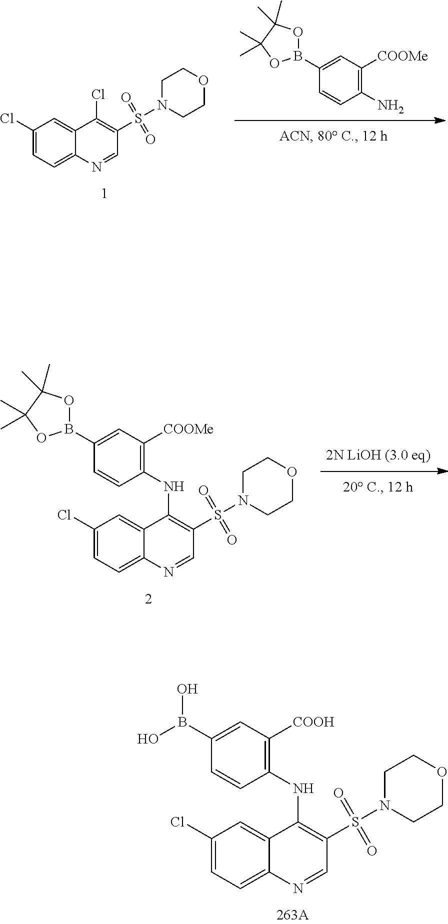 [SMILES: O=S(=O)(c1cnc2ccc(Cl)cc2c1Cl)N1CCOCC1 ; COC(=O)c1cc(B2OC(C)(C)C(C)(C)O2)ccc1N ; COC(=O)c1cc(B2OC(C)(C)C(C)(C)O2)ccc1Nc1c(S(=O)(=O)N2CCOCC2)cnc2ccc(Cl)cc12 ; O=C(O)c1cc(B(O)O)ccc1Nc1c(S(=O)(=O)N2CCOCC2)cnc2ccc(Cl)cc12]

Step 1. Synthesis of methyl 2-[(6-chloro-3-morpholinosulfonyl-4-quinolyl)amino]-5-(4,4,5,5-tetramethyl-1,3,2-dioxaborolan-2-yl)benzoate (2): A solution of 4-[(4,6-dichloro-3-quinolyl)sulfonyl]morpholine (200 mg, 576.01 umol, 1 eq) and methyl 2-amino-5-(4,4,5,5-tetramethyl-1,3,2-dioxaborolan-2-yl)benzoate (159.63 mg, 576.01 umol, 1 eq) in ACN (4 mL) was stirred at 80° C. for 12 h. LCMS showed the starting material was consumed completely and desired MS was detected. The reaction mixture was filtered, and filter cake was concentrated in vacuum. Compound methyl 2-[(6-chloro-3-morpholinosulfonyl-4-quinolyl)amino]-5-(4,4,5,5-tetramethyl-1,3,2-dioxaborolan-2-yl)benzoate (280 mg, 476.29 umol, 82.69% yield) was obtained as a yellow solid. MS (M+H)⁺=588.2.

Step 2. Synthesis of 5-borono-2-[(6-chloro-3-morpholinosulfonyl-4-quinolyl)amino]benzoic acid (263A): To a stirred solution of methyl 2-[(6-chloro-3-morpholinosulfonyl-4-quinolyl)amino]-5-(4,4,5,5-tetramethyl-1,3,2-dioxaborolan-2-yl)benzoate (100 mg, 170.10 umol, 1 eq) in THF (1 mL) was added LiOH (2 M, 255.15 uL, 3 eq) at 20° C., the mixture was stirred at 20° C. for 12 h. LCMS showed the starting material was consumed completely and desired MS was detected. The reaction mixture was adjusted pH˜4 by adding 2N HCl. Then the mixture was concentrated in vacuum. The residue was purified by prep-HPLC (column: Phenomenex Luna 80*30 mm*3 um; mobile phase: [water(0.04% HCl)-ACN]; B %: 15%-35%,8 min). Compound 5-borono-2-[(6-chloro-3-morpholinosulfonyl-4-quinolyl)amino]benzoic acid (9.10 mg, 17.23 umol, 10.13% yield, 100% purity, HCl) was obtained as a yellow solid. ¹H NMR (400 MHz, DMSO-d₆) δ=10.54 (br s, 1H), 9.11 (s, 1H), 8.49 (d, J=1.5 Hz, 1H), 8.15 (d, J=9.0 Hz, 1H), 7.92 (dd, J=2.3, 9.0 Hz, 1H), 7.70 (dd, J=1.5, 8.3 Hz, 1H), 7.62 (d, J=2.4 Hz, 1H), 6.61 (d, J=8.4 Hz, 1H), 3.35-3.29 (m, 4H), 3.10-2.96 (m, 4H). MS (M+H)⁺=492.0.

### Example 67—Synthesis of 265A

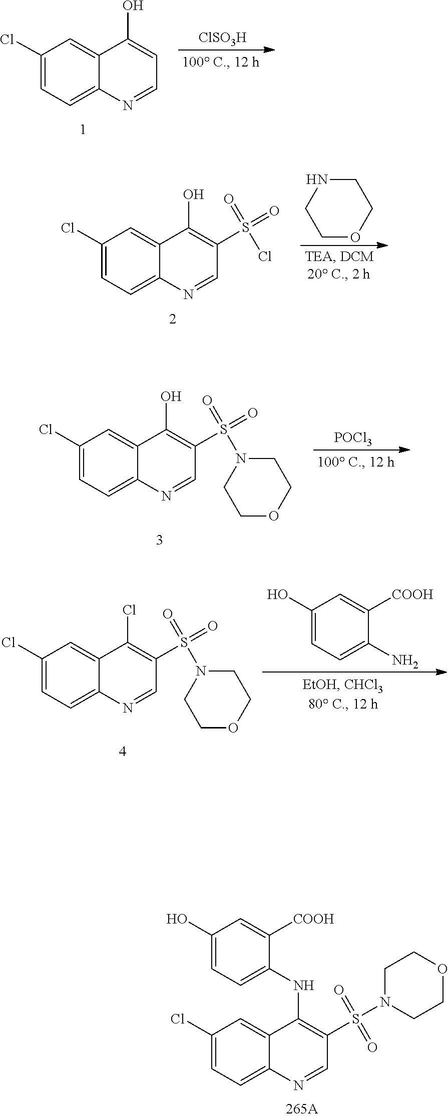

Step 1. Synthesis of 6-chloro-4-hydroxy-quinoline-3-sulfonyl chloride (2): A solution of 6-chloroquinolin-4-ol (3 g, 16.70 mmol, 1 eq) in HSO₃Cl (20 mL), the mixture was purged with N₂, the reaction was stirred at 100° C. for 12 h. TLC (PE:EtOAc=1:1, R_{f}=0.18) showed the starting material was consumed completely and new spot was formed. The mixture was quenched in H₂O, then the reaction was filtered, the filter cake was concentrated in vacuum. Compound 6-chloro-4-hydroxy-quinoline-3-sulfonyl chloride (5 g, crude) was obtained as a white solid.

Step 2. Synthesis of 6-chloro-3-morpholinosulfonyl-quinolin-4-ol (3): To a stirred solution of 6-chloro-4-hydroxy-quinoline-3-sulfonyl chloride (5 g, 17.98 mmol, 1 eq) in DCM (50 mL) was added morpholine (1.72 g, 19.78 mmol, 1.74 mL, 1.1 eq) TEA (3.64 g, 35.96 mmol, 5.00 mL, 2 eq) the mixture was stirred at 20° C. for 2 h. LCMS showed starting material was consumed completely and the MS of desired product was detected. The reaction mixture was concentrated in vacuum. Compound 6-chloro-3-morpholinosulfonyl-quinolin-4-ol (5. g, 15.21 mmol, 84.59% yield) was obtained as off-white solid. MS (M+H)⁺=329.1

Step 3. Synthesis of 4-[(4,6-dichloro-3-quinolyl)sulfonyl]morpholine (4): A solution of 6-chloro-3-morpholinosulfonyl-quinolin-4-ol (5 g, 15.21 mmol, 1 eq) in POCl₃ (30 mL), the mixture was purged with N₂, the reaction was stirred at 100° C. for 12 h. LCMS (showed starting material was consumed completely and the MS of desired product was detected. TLC (Petroleum ether/Ethyl acetate=3:1, R_{f}=0.48) showed starting material was consumed completely and new spot was formed. The reaction mixture was cooled to room temperature and quenched by water (30 mL), extracted with ethyl acetate (30 mL*2). The combined organics were washed with brine (25 mL), dried over Na₂SO₄, filtered and concentrated under reduced pressure to give a residue. The residue was purified by flash column (ISCO 40 g silica, 50-70% ethyl acetate in petroleum ether, gradient over 40 min). Compound 4-[(4,6-dichloro-3-quinolyl) sulfonyl]morpholine (1.7 g, 4.90 mmol, 32.19% yield) was obtained as a white solid. ¹H NMR (400 MHz, DMSO-d6) δ ppm 9.23 (s, 1H), 8.46 (d, J=2.32 Hz, 1H), 8.24 (d, J=8.92 Hz, 1H), 8.09 (dd, J=9.05, 2.32 Hz, 1H), 3.56-3.68 (m, 4H), 3.22-3.32 (m, 4H). MS (M+H)⁺=347.1

Step 4. Synthesis of 2-[(6-chloro-3-morpholinosulfonyl-4-quinolyl)amino]-5-hydroxy-benzoic acid (265A): To a solution of 4-[(4,6-dichloro-3-quinolyl)sulfonyl]morpholine (50 mg, 144.00 umol, 1 eq) in CHCl3 (0.1 mL) and EtOH (0.5 mL) was added 2-amino-5-hydroxy-benzoic acid (22.05 mg. 144.00 umol, 1 eq), the reaction was stirred at 80° C. for 12 h. LCMS showed starting material was consumed completely and the MS of desired product was detected. The reaction was concentrated in vacuum. The crude product was purified by prep-HPLC (column: Phenomenex Luna 80*30 mm*3 um; mobile phase: [water (0.04% HCl)-ACN]; B %: 5%-35%,8 min) Compound 2-[(6-chloro-3-morpholinosulfonyl-4-quinolyl)amino]-5-hydroxy-benzoic acid (41.9 mg, 82.64 umol, 57.38% yield, 98.68% purity, HCl) was obtained as orange solid. ¹H NMR (400 MHz, DMSO-d6) δ ppm 10.28 (br s, 1H) 9.02 (s, 1H) 8.10 (d, J=8.92 Hz, 1H) 7.89-7.96 (m, 1H) 7.44 (dd, J=11.80, 2.51 Hz, 2H) 6.94 (br d, J=8.68 Hz, 1H) 6.85-6.91 (m, 1H) 3.58 (br d, J=3.67 Hz, 2H) 3.50-3.54 (m, 2H) 3.03-3.20 (m, 4H). MS (M+H)⁺=464.0

### Example 68—Synthesis of 266A

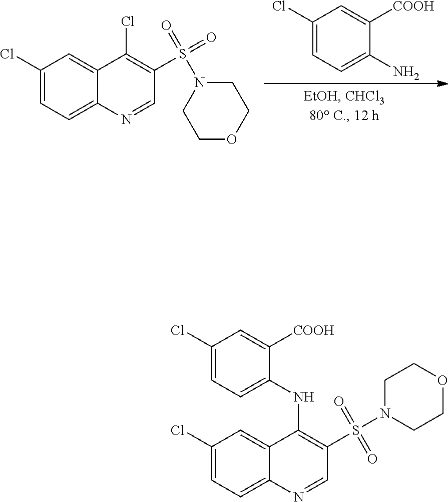 [SMILES: O=S(=O)(c1cnc2ccc(Cl)cc2c1Cl)N1CCOCC1 ; Nc1ccc(Cl)cc1C(=O)O ; O=C(O)c1cc(Cl)ccc1Nc1c(S(=O)(=O)N2CCOCC2)cnc2ccc(Cl)cc12]

To a solution of 4-[(4,6-dichloro-3-quinolyl)sulfonyl]morpholine (50 mg, 144.00 umol, 1 eq) in CHCl₃ (0.1 mL) and EtOH (0.5 mL) was added 2-amino-5-chloro-benzoic acid (24.71 mg, 144.00 umol, 1 eq), the reaction was stirred at 80° C. for 12 h. LCMS showed starting material was consumed completely and the MS of desired product was detected. The reaction was concentrated in vacuum. The crude product was purified by prep-HPLC (column: Phenomenex Luna 80*30 mm*3 um; mobile phase: [water(0.04% HCl)-ACN]; B %: 20%-50%,8 min) Compound 5-chloro-2-[(6-chloro-3-morpholinosulfonyl-4-quinolyl)amino]benzoic acid (33.8 mg, 65.15 umol, 45.24% yield, 100% purity, HCl) was obtained as a yellow solid. ¹H NMR (400 MHz, DMSO-d6) δ ppm 10.41 (br s, 1H), 9.09 (s, 1H), 8.14 (d, J=9.05 Hz, 1H), 7.86-7.99 (m, 2H), 7.64 (d, J=2.20 Hz, 1H), 7.39 (dd, J=8.86, 2.63 Hz, 1H), 6.70 (d, J=8.92 Hz, 1H), 3.51-3.52 (m, 2H), 3.35-3.38 (m, 2H), 2.97-3.08 (m, 4H). MS (M+H)⁺=482.0

### Example 69—Synthesis of 267A

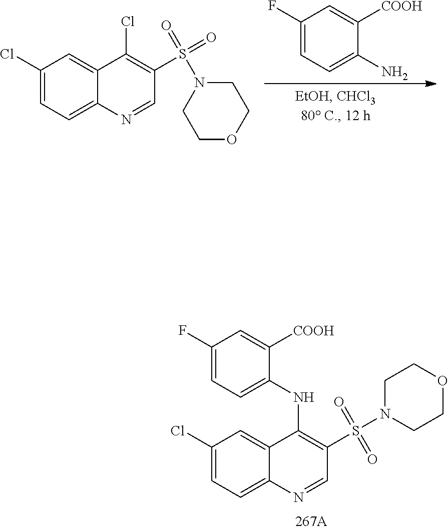 [SMILES: O=S(=O)(c1cnc2ccc(Cl)cc2c1Cl)N1CCOCC1 ; Nc1ccc(F)cc1C(=O)O ; O=C(O)c1cc(F)ccc1Nc1c(S(=O)(=O)N2CCOCC2)cnc2ccc(Cl)cc12]

To a solution of 4-[(4,6-dichloro-3-quinolyl)sulfonyl]morpholine (50 mg, 144.00 umol, 1 eq) in CHCl₃ (0.1 mL) and EtOH (0.5 mL) was added 2-amino-5-fluoro-benzoic acid (22.34 mg, 144.00 umol, 1 eq), the reaction was stirred at 80° C. for 12 h. LCMS showed starting material was consumed completely and the MS of desired product was detected. The reaction was concentrated in vacuum. The crude product was purified by prep-HPLC (column: Phenomenex Luna 80*30 mm*3 um; mobile phase: [water(0.04% HCl)-ACN]; B %: 15%-55%,8 min) Compound 2-[(6-chloro-3-morpholinosulfonyl-4-quinolyl)amino]-5-fluoro-benzoic acid (26.8 mg, 53.35 umol, 37.05% yield, 100% purity, HCl) was obtained as a yellow solid. ¹H NMR (400 MHz, DMSO-d6+D₂O) δ ppm 9.09 (s, 1H), 8.12 (d, J=9.01 Hz, 1H), 7.93 (dd, J=9.07, 1.81 Hz, 1H), 7.75 (dd, J=9.01, 3.13 Hz, 1H), 7.54 (d, J=1.63 Hz, 1H), 7.24-7.35 (m, 1H) 6.92 (br dd, J=8.88, 4.50 Hz, 1H) 3.49-3.57 (m, 2H) 3.38-3.47 (m, 2H) 2.99-3.15 (m, 4H). MS (M+H)⁺=466.0

### Example 70—Synthesis of Compound 268A

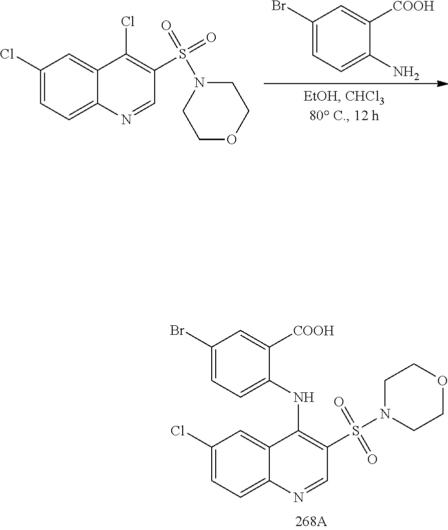 [SMILES: O=S(=O)(c1cnc2ccc(Cl)cc2c1Cl)N1CCOCC1 ; Nc1ccc(Br)cc1C(=O)O ; O=C(O)c1cc(Br)ccc1Nc1c(S(=O)(=O)N2CCOCC2)cnc2ccc(Cl)cc12]

To a solution of 4-[(4,6-dichloro-3-quinolyl)sulfonyl]morpholine (30 mg, 86.40 umol, 1 eq) CHCl₃ (0.1 mL) and EtOH (0.5 mL) was added 2-amino-5-bromo-benzoic acid (18.67 mg, 86.40 umol, 1 eq), the mixture was stirred at 80° C. for 2 h. LC-MS showed starting material was consumed completely and the MS of desired product was detected. The reaction mixture was concentrated in vacuum. The residue was purified by prep-HPLC (column: Phenomenex Luna C18 75*30 mm*3 um; mobile phase: [water(0.04% HCl)-ACN]; B %: 10%-40%,8 min). Compound 5-bromo-2-[(6-chloro-3-morpholinosulfonyl-4-quinolyl)amino]benzoic acid (6.2 mg, 10.64 umol, 12.31% yield, 96.66% purity, HCl) was obtained as a yellow solid. ¹H NMR (400 MHz, DMSO-d6+D₂O) δ ppm 8.87 (s, 1H), 8.00 (d, J=9.13 Hz, 1H), 7.82 (dd, J=8.94, 1.94 Hz, 1H), 7.46 (d, J=7.88 Hz, 1H), 7.39 (d, J=1.88 Hz, 1H), 7.19 (t, J=8.07 Hz, 1H), 6.79 (d, J=8.13 Hz, 1H), 3.39-3.61 (m, 4H), 2.93-3.16 (m, 4H). MS (M+H)⁺=527.9.

### Example 71—Synthesis of Compound 269A

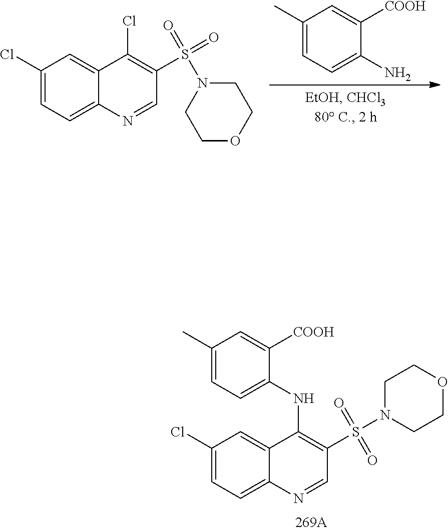 [SMILES: O=S(=O)(c1cnc2ccc(Cl)cc2c1Cl)N1CCOCC1 ; Cc1ccc(N)c(C(=O)O)c1 ; Cc1ccc(Nc2c(S(=O)(=O)N3CCOCC3)cnc3ccc(Cl)cc23)c(C(=O)O)c1]

To a solution of 4-[(4,6-dichloro-3-quinolyl)sulfonyl]morpholine (30 mg, 86.40 umol, 1 eq) in CHCl₃ (0.2 mL) and EtOH (1 mL) was added 2-amino-5-methyl-benzoic acid (13.06 mg, 86.40 umol, 1 eq), the mixture was stirred at 80° C. for 2 h. LC-MS showed starting material was consumed completely and the Ms of desired product was detected. The reaction mixture was concentrated in vacuum. The residue was purified by prep-HPLC (column: Phenomenex Luna C18 75*30 mm*3 um; mobile phase:[water(0.04% HCl)-ACN]; B %:15%-45%,8 min). Compound 2-[(6-chloro-3-morpholinosulfonyl-4-quinolyl)amino]-5-methyl-benzoic acid (6.3 mg, 12.64 umol, 14.63% yield, 100% purity, HCl) was obtained as a yellow solid. ¹H NMR (400 MHz, DMSO-d6) δ ppm 10.33 (br s, 1H), 9.07 (s, 1H), 8.12 (d, J=9.01 Hz, 1H), 7.90 (dd, J=9.01, 2.38 Hz, 1H), 7.82 (d, J=1.75 Hz, 1H), 7.57 (d, J=2.25 Hz, 1H), 7.20 (dd, J=8.38, 1.88 Hz, 1H), 6.69 (d, J=8.38 Hz, 1H), 3.47-3.56 (m, 2H), 3.35-3.44 (m, 2H), 2.97-3.13 (m, 4H), 2.30 (s, 3H). MS (M+H)⁺=462.0.

### Example 72—Synthesis of Compound 270A

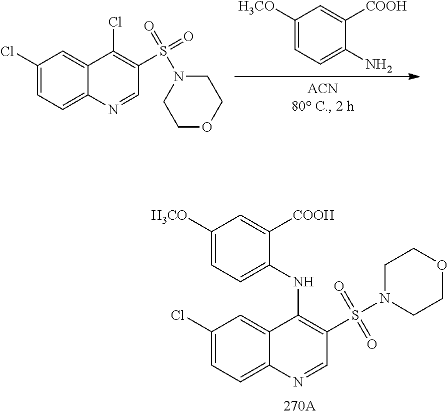 [SMILES: O=S(=O)(c1cnc2ccc(Cl)cc2c1Cl)N1CCOCC1 ; COc1ccc(N)c(C(=O)O)c1 ; COc1ccc(Nc2c(S(=O)(=O)N3CCOCC3)cnc3ccc(Cl)cc23)c(C(=O)O)c1]

A solution of 4-[(4,6-dichloro-3-quinolyl)sulfonyl]morpholine (30 mg, 86.40 umol, 1 eq) and 2-amino-5-methoxy-benzoic acid (14.44 mg, 86.40 umol, 1 eq) in ACN (1 mL) was stirred at 80° C. for 2 h. LC-MS showed starting material was consumed completely and the Ms of desired product was detected. The reaction mixture was concentrated in vacuum. The residue was purified by prep-HPLC (column: Phenomenexluna C1880*40 mm*3 um; mobile phase: [water (0.04% HCl)-ACN]; B %:28%-55%,7 min). Compound 2-[(6-chloro-3-morpholinosulfonyl-4-quinolyl)amino]-5-methoxy-benzoic acid (15.0 mg, 29.16 umol, 33.75% yield, 100% purity, HCl) was obtained as a yellow solid. ¹H NMR (400 MHz, DMSO-d6) δ ppm 10.24 (br s, 1H),9.03 (s, 1H),8.10 (d, J=9.00 Hz, 1H),7.90 (dd, J=8.94, 2.19 Hz, 1H),7.50 (d, J=2.63 Hz, 2H), 7.04 (dd, J=8.94, 3.06 Hz, 1H),6.90 (br d, J=9.01 Hz, 1H), 3.80 (s, 3H), 3.52-3.59 (m, 2H),3.42-3.50 (m, 2H), 3.01-3.15 (m, 4H). MS (M+H)⁺=478.0.

### Example 73—Synthesis of Compound 271A

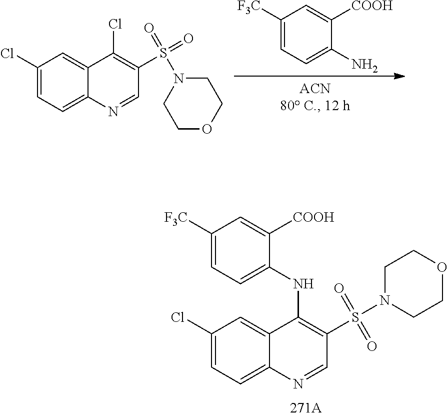 [SMILES: O=S(=O)(c1cnc2ccc(Cl)cc2c1Cl)N1CCOCC1 ; Nc1ccc(C(F)(F)F)cc1C(=O)O ; O=C(O)c1cc(C(F)(F)F)ccc1Nc1c(S(=O)(=O)N2CCOCC2)cnc2ccc(Cl)cc12]

To a solution of 4-[(4,6-dichloro-3-quinolyl)sulfonyl]morpholine (100 mg, 288.00 umol, 1 eq) and 2-amino-5-(trifluoromethyl)benzoic acid (59.08 mg, 288.00 umol, 1 eq) in ACN (3 mL) was stirred at 80° C. for 12 h. LC-MS showed starting material was consumed completely and the Ms of desired product was detected. The reaction mixture was concentrated in vacuum. The residue was purified by prep-HPLC (column: Phenomenex luna C18 80*40 mm*3 um; mobile phase: [water(0.04% HCl)-ACN]; B %:40%-70%,7 min). Compound 2-[(6-chloro-3-morpholinosulfonyl-4-quinolyl)amino]-5-(trifluoromethyl)benzoic acid (7.9 mg, 13.73 umol, 4.77% yield, 95.98% purity, HCl) was obtained as a yellow solid. ¹H NMR (400 MHz, DMSO-d6) δ ppm 10.71 (br s, 1H), 9.16 (s, 1H), 8.23 (s, 1H), 8.19 (d, J=9.05 Hz, 1H), 7.95 (dd, J=9.05, 2.32 Hz, 1H), 7.73 (d, J=2.20 Hz, 1H), 7.63 (dd, J=8.86, 2.14 Hz, 1H), 6.72 (d, J=8.80 Hz, 1H), 3.44-3.47 (m, 2H), 3.36 (br t, J=4.77 Hz, 2H), 3.03 (t, J=4.52 Hz, 4H). MS (M+H)⁺=516.0

### Example 74—Synthesis of Compound 272A

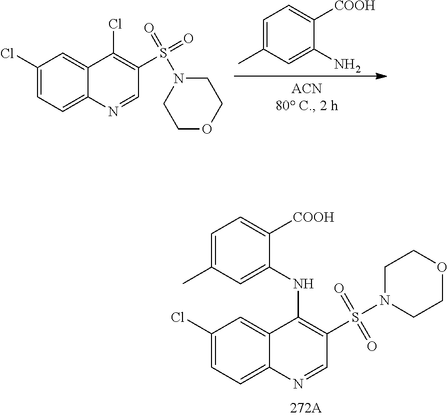

To a solution of 4-[(4,6-dichloro-3-quinolyl)sulfonyl]morpholine (30 mg, 86.40 umol, 1 eq) in ACN (1 mL) was added 2-amino-4-methyl-benzoic acid (13.06 mg, 86.40 umol, 1 eq), the mixture was stirred at 80° C. for 2 h. LC-MS showed starting material was consumed completely and the Ms of desired product was detected. he reaction mixture was concentrated in vacuum. he residue was purified by prep-HPLC (column: Phenomenex luna C18 80*40 mm*3 um; mobile phase: [water(0.04% HCl)-ACN]; B %: 36%-55%,7 min). Compound 2-[(6-chloro-3-morpholinosulfonyl-4-quinolyl)amino]-4-methyl-benzoic acid (12.9 mg, 27.68 umol, 32.03% yield,99.1% purity) was obtained as a yellow solid. ¹H NMR (400 MHz, DMSO-d6) δ ppm 10.44 (br s, 1H), 9.10 (s, 1H), 8.14 (d, J=9.01 Hz, 1H), 7.87-7.95 (m, 2H),7.58 (d, J=2.25 Hz, 1H), 6.93 (d, J=8.13 Hz, 1H), 6.58 (s, 1H), 3.43-3.53 (m, 2H), 3.31-3.41 (m, 2H), 2.99-3.13 (m, 4H), 2.10 (s, 3H). MS (M+H)⁺=462.0

### Example 75—Synthesis of Compound 273A

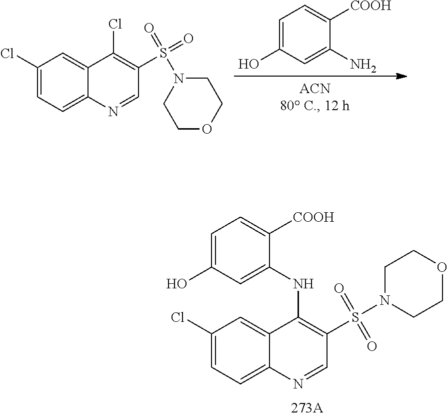 [SMILES: O=S(=O)(c1cnc2ccc(Cl)cc2c1Cl)N1CCOCC1 ; Nc1cc(O)ccc1C(=O)O ; O=C(O)c1ccc(O)cc1Nc1c(S(=O)(=O)N2CCOCC2)cnc2ccc(Cl)cc12]

A solution of 4-[(4,6-dichloro-3-quinolyl)sulfonyl]morpholine (40 mg, 115.20 umol, 1 eq) and 2-amino-4-hydroxy-benzoic acid (17.64 mg, 115.20 umol, 1 eq) in ACN (1 mL) was stirred at 80° C. for 12 h. LCMS showed the starting material was consumed completely and desired MS was detected. The reaction mixture was concentrate in vacuum. The residue was purified by prep-HPLC (column: Phenomenex Luna 80*30 mm*3 um; mobile phase: [water(0.04% HCl)-ACN]; B %: 15%-40%,8 min). Compound 2-[(6-chloro-3-morpholinosulfonyl-4-quinolyl)amino]-4-hydroxy-benzoic acid (16.3 mg, 32.58 umol, 28.28% yield, 100% purity, HCl) was obtained as a yellow solid. ¹H NMR (400 MHz, DMSO-d6) δ=10.50 (br s, 1H), 10.31-10.04 (m, 1H), 9.12 (s, 1H), 8.16 (d, J=9.0 Hz, 1H), 7.95 (dd, J=2.4, 9.0 Hz, 1H), 7.86 (d, J=8.8 Hz, 1H), 7.68 (d, J=2.0 Hz, 1H), 6.48 (dd, J=2.1, 8.7 Hz, 1H), 5.93 (s, 1H), 3.53-3.32 (m, 4H), 3.13-2.97 (m, 4H). MS (M+H)⁺=464.0

### Example 76—Synthesis of Compound 274A

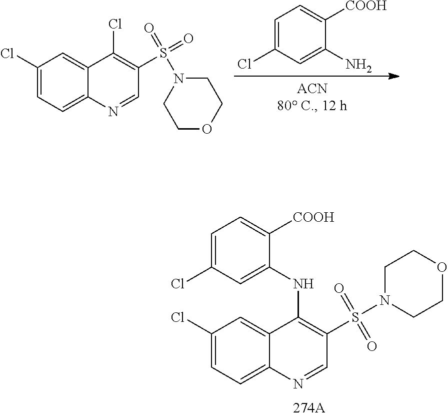 [SMILES: O=S(=O)(c1cnc2ccc(Cl)cc2c1Cl)N1CCOCC1 ; Nc1cc(Cl)ccc1C(=O)O ; O=C(O)c1ccc(Cl)cc1Nc1c(S(=O)(=O)N2CCOCC2)cnc2ccc(Cl)cc12]

To a solution of 4-[(4,6-dichloro-3-quinolyl)sulfonyl]morpholine (30 mg, 86.40 umol, 1 eq) and 2-amino-4-chloro-benzoic acid (14.82 mg, 86.40 umol, 1 eq) in ACN (1 mL) was stirred at 80° C. for 12 h. LC-MS showed starting material was consumed completely and the Ms of desired product was detected. The reaction mixture was concentrated in vacuum. The residue was purified by prep-HPLC (column: Phenomenex Luna 80*30 mm*3 um; mobile phase: [water (0.04% HCl)-ACN]; B %: 40%-70%,8 min). Compound 4-chloro-2-[(6-chloro-3-morpholinosulfonyl-4-quinolyl)amino]benzoic acid (17.5 mg, 33.18 umol, 38.40% yield, 98.36% purity, HCl) was obtained as a yellow solid. ¹H NMR (400 MHz, DMSO-d6) δ ppm 10.37-10.62 (m, 1H), 9.09-9.13 (m, 1H), 8.14-8.19 (m, 1H), 7.97-8.01 (m, 1H), 7.92-7.97 (m, 1H), 7.66 (d, J=2.25 Hz, 1H), 7.08 (dd, J=8.57, 1.69 Hz, 1H), 6.76 (s, 1H), 3.44-3.46 (m, 2H), 3.33-3.37 (m, 2H), 3.02-3.11 (m, 4H). MS (M+H)⁺=481.9

### Example 77—Synthesis of Compound 275A

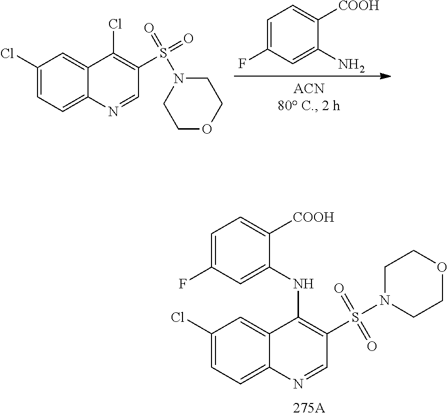

To a solution of 4-[(4,6-dichloro-3-quinolyl)sulfonyl]morpholine (30 mg, 86.40 umol, 1 eq) and 2-amino-4-fluoro-benzoicacid (13.40 mg, 86.40 umol, 1 eq) in ACN (1 mL) was stirred at 80° C. for 2 h. LC-MS showed starting material was consumed completely and the Ms of desired product was detected. The reaction mixture was concentrated in vacuum. The residue was purified by prep-HPLC (column: Phenomenex Luna 80*30 mm*3 um; mobile phase: [water(0.04% HCl)-ACN]; B %: 35%-60%,8 min). Compound 2-[(6-chloro-3-morpholinosulfonyl-4-quinolyl)amino]-4-fluoro-benzoic acid (14 mg, 30.05 umol, 34.78% yield, 100% purity) was obtained as a yellow solid. ¹H NMR (400 MHz, DMSO-d6) δ ppm 10.59 (br s, 1H), 9.12 (s, 1H), 8.17 (d, J=9.01 Hz, 1H), 8.07 (dd, J=8.82, 6.82 Hz, 1H), 7.95 (dd, J=9.01, 2.25 Hz, 1H), 7.67 (d, J=2.25 Hz, 1H), 6.86 (td, J=8.44, 2.38 Hz, 1H), 6.42-6.58 (m, 1H), 3.47 (br s, 2H), 3.34-3.40 (m, 2H), 2.99-3.12 (m, 4H). MS (M+H)⁺=466.0.

### Example 78—Synthesis of Compound 276A

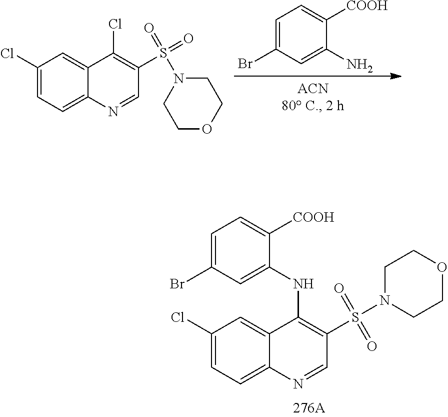 [SMILES: O=S(=O)(c1cnc2ccc(Cl)cc2c1Cl)N1CCOCC1 ; Nc1cc(Br)ccc1C(=O)O ; O=C(O)c1ccc(Br)cc1Nc1c(S(=O)(=O)N2CCOCC2)cnc2ccc(Cl)cc12]

To a solution of 4-[(4,6-dichloro-3-quinolyl)sulfonyl]morpholine (30 mg, 86.40 umol, 1 eq) and 2-amino-4-bromo-benzoic acid (18.67 mg, 86.40 umol, 1 eq) in ACN (1 mL) was stirred at 80° C. for 2 h. LC-MS showed starting material was consumed completely and the Ms of desired product was detected. The reaction mixture was concentrated in vacuum. The residue was purified by prep-HPLC (column: Phenomenex Luna 80*30 mm*3 um; mobile phase: [water(0.04% HCl)-ACN]; B %: 40%-65%,8 min). Compound 4-bromo-2-[(6-chloro-3-morpholinosulfonyl-4-quinolyl)amino]benzoic acid (14.8 mg, 26.28 umol, 30.41% yield, 100% purity, HCl) was obtained as a yellow solid. ¹H NMR (400 MHz, DMSO-d6) δ ppm 10.51 (br s, 1H), 9.11 (s, 1H), 8.17 (d, J=8.92 Hz, 1H), 7.95 (dd, J=9.05, 2.32 Hz, 1H), 7.91 (d, J=8.44 Hz, 1H), 7.66 (d, J=2.20 Hz, 1H), 7.22 (dd, J=8.56, 1.83 Hz, 1H), 6.90 (d, J=1.71 Hz, 1H), 3.49 (br s, 2H), 3.31-3.35 (m, 2H), 3.07 (br s, 4H). MS (M+H)⁺=527.9

### Example 79—Synthesis of Compound 277A

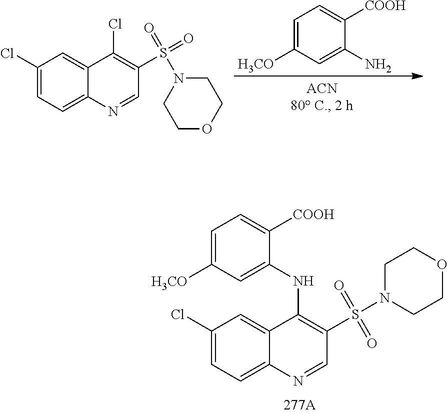 [SMILES: O=S(=O)(c1cnc2ccc(Cl)cc2c1Cl)N1CCOCC1 ; COc1ccc(C(=O)O)c(N)c1 ; COc1ccc(C(=O)O)c(Nc2c(S(=O)(=O)N3CCOCC3)cnc3ccc(Cl)cc23)c1]

To a solution of 4-[(4,6-dichloro-3-quinolyl)sulfonyl]morpholine (40 mg, 115.20 umol, 1 eq) and 2-amino-4-methoxy-benzoic acid (19.26 mg, 115.20 umol, 1 eq) in ACN (1.5 mL) was stirred at 80° C. for 2 h. LC-MS showed starting material was consumed completely and the Ms of desired product was detected. The reaction mixture was concentrated in vacuum. The residue was purified by prep-HPLC (column: Phenomenex Luna 80*30 mm*3 um; mobile phase: [water(0.04% HCl)-ACN]; B %: 20%-50%,8 min). Compound 2-[(6-chloro-3-morpholinosulfonyl-4-quinolyl)amino]-4-methoxy-benzoic acid (20.9 mg, 40.63 umol, 35.27% yield, 100% purity, HCl) was obtained as a yellow solid. ¹H NMR (400 MHz, DMSO-d6) δ ppm 10.55 (s, 1H), 9.10 (s, 1H), 8.14 (d, J=9.00 Hz, 1H), 7.96 (d, J=8.88 Hz, 1H), 7.91(dd, J=9.01, 2.38 Hz, 1H), 7.66 (d, J=2.25 Hz, 1H), 6.64 (dd, J=8.88, 2.38 Hz, 1H), 6.09 (d, J=2.38 Hz, 1H), 3.53 (s, 3H), 3.45-3.50 (m, 2H), 3.31-3.34 (m, 2H), 2.96-3.14 (m, 4H). MS (M+H)⁺=478.0.

### Example 80—Synthesis of Compound 278A

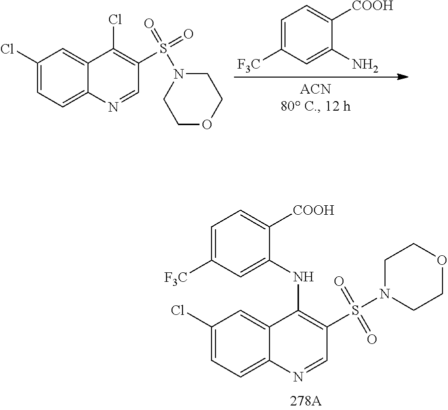 [SMILES: O=S(=O)(c1cnc2ccc(Cl)cc2c1Cl)N1CCOCC1 ; Nc1cc(C(F)(F)F)ccc1C(=O)O ; O=C(O)c1ccc(C(F)(F)F)cc1Nc1c(S(=O)(=O)N2CCOCC2)cnc2ccc(Cl)cc12]

To a solution of 4-[(4,6-dichloro-3-quinolyl)sulfonyl]morpholine (40 mg, 115.20 umol, 1 eq) and 2-amino-4-(trifluoromethyl)benzoic acid (23.63 mg, 115.20 umol, 1 eq) in ACN (1 mL) was stirred at 80° C. for 12 h. LC-MS showed starting material was consumed completely and the Ms of desired product was detected. The reaction mixture was concentrated in vacuum. The residue was purified by prep-HPLC (column: Phenomenex luna C18 80*40 mm*3 um; mobile phase: [water(0.04% HCl)-ACN]; B %: 45%-73%,7 min). Compound 2-[(6-chloro-3-morpholinosulfonyl-4-quinolyl)amino]-4-(trifluoromethyl)benzoic acid (14.3 mg, 25.89 umol, 22.47% yield, 100% purity, HCl) was obtained as a yellow solid. ¹H NMR (400 MHz, DMSO-d6) δ ppm 10.67 (br d, J=1.50 Hz, 1H), 9.12 (s, 1H), 8.18 (dd, J=11.44, 8.69 Hz, 2H), 7.94 (dd, J=8.94, 2.31 Hz, 1H), 7.62 (d, J=2.25 Hz, 1H), 7.37 (dd, J=8.32, 1.06 Hz, 1H), 7.02 (s, 1H), 3.43-3.49 (m, 2H), 3.28-3.36 (m, 2H), 3.01-3.15 (m, 4H). MS (M+H)⁺=516.0

### Example 81—Synthesis of Compound 279A

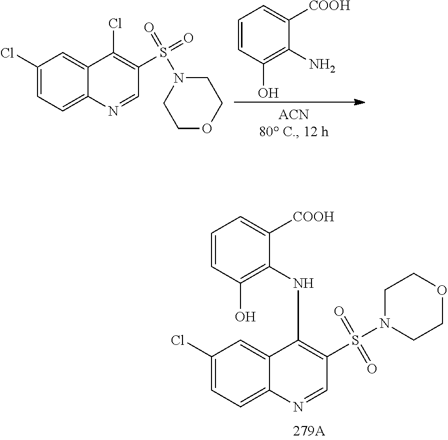

To a solution of 4-[(4,6-dichloro-3-quinolyl)sulfonyl]morpholine (40 mg, 115.20 umol, 1 eq) and 2-amino-3-hydroxy-benzoic acid (17.64 mg, 115.20 umol, 1 eq) in ACN (1.5 mL) w as stirred at 80° C. for 12 h. LC-MS showed starting material was consumed completely and the Ms of desired product was detected. The reaction mixture was concentrated in vacuum. The residue was purified by prep-HPLC (column: Phenomenex luna C18 80*40 mm*3 um; mobile phase: [water(0.04% HCl)-ACN]; B %: 23%-48%,7 min). Compound 2-[(6-chloro-3-morpholinosulfonyl-4-quinolyl)amino]-3-hydroxy-benzoic acid (16.3 mg, 32.58 umol. 28.28% yield,100% purity, HCl) was obtained as a yellow solid. ¹H NMR (400 MHz, DMSO-d6) δ ppm 10.56 (br s, 1H), 10.27 (br s, 1H), 9.00 (s, 1H), 8.09-8.17 (m, 1H), 7.92-7.98 (m, 1H), 7.53 (dd, J=7.75, 1.25 Hz, 1H), 7.46 (d, J=2.13 Hz, 1H), 7.30 (t, J=7.94 Hz, 1H), 7.15 (br d, J=8.13 Hz, 1H), 3.60 (br t, J=5.50 Hz, 4H),3.21-3.29 (m, 2H), 3.12-3.20 (m, 2H). MS (M+H)⁺=464.0

### Example 82—Synthesis of 280A

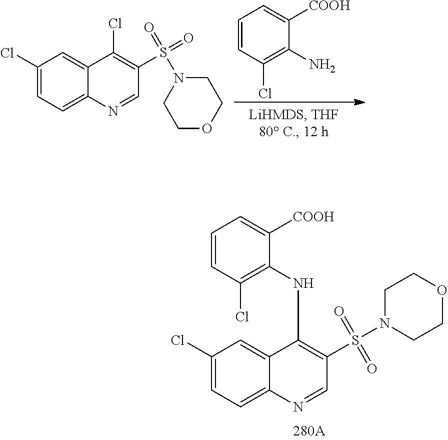

To a solution of 4-[(4,6-dichloro-3-quinolyl)sulfonyl]morpholine (50 mg, 144.00 umol, 1 eq) in THF (0.5 mL) was added 2-amino-3-chloro-benzoic acid (24.71 mg, 144.00 umol, 1 eq) and LiHMDS (1 M, 216.00 uL, 1.5 eq), the mixture was purged with N₂ for 3 times, the reaction was stirred at 80° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water (0.04% HCl)-ACN]; B %: 20%-50%,8 min) Compound 3-chloro-2-[(6-chloro-3-morpholinosulfonyl-4-quinolyl)amino]benzoic acid (7.3 mg, 13.88 umol, 9.64% yield, 98.66% purity, HCl) was obtained as a yellow solid. ¹H NMR (400 MHz, DMSO-d6) δ ppm 8.58 (br s, 1H), 7.89 (br d, J=7.70 Hz, 1H), 7.75 (br s, 1H), 7.66-7.73 (m, 2H), 7.16-7.26 (m, 1H), 7.12 (d, J=2.08 Hz, 1H), 3.58 (br d, J=4.40 Hz, 4H) 3.20 (br s, 4H). MS (M+H)⁺=482.0

### Example 83—Synthesis of 281A

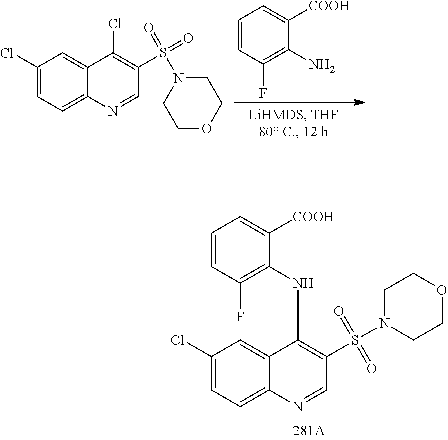

To a solution of 2-amino-3-fluoro-benzoic acid (17.87 mg, 115.20 umol, 1 eq) in THF (1.5 mL) was added dropwise LiHMDS (1 M, 345.61 uL, 3 eq) and 4-[(4,6-dichloro-3-quinolyl)sulfonyl]morpholine (40 mg, 115.20 umol, 1 eq), the mixture was purged with N₂, the reaction was stirred at 80° C. for 12 h. LCMS showed starting material was consumed completely and the MS of desired product was detected. The mixture was dissolved by MeOH (3 ml), then the mixture was concentrated in vacuum. The crude product was purified by prep-HPLC (column: Phenomenex Luna 80*30 mm*3 um; mobile phase: [water(0.04% HCl)-ACN]; B %: 15%-45%,8 min) Compound 2-[(6-chloro-3-morpholinosulfonyl-4-quinolyl)amino]-3-fluoro-benzoic acid (7.4 mg, 14.19 umol, 12.32% yield, 96.32% purity, HCl) was obtained as a yellow solid. ¹H NMR (400 MHz, DMSO-d6) δ ppm 8.90 (br s, 1H), 8.02 (br d, J=9.05 Hz, 1H), 7.79-7.95 (m, 2H), 7.37-7.53 (m, 2H) 7.27 (br s, 1H), 3.58 (br d, J=7.58 Hz, 4H), 3.11 (br s, 4H). MS (M+H)⁺=466.0

### Example 84—Synthesis of 282A

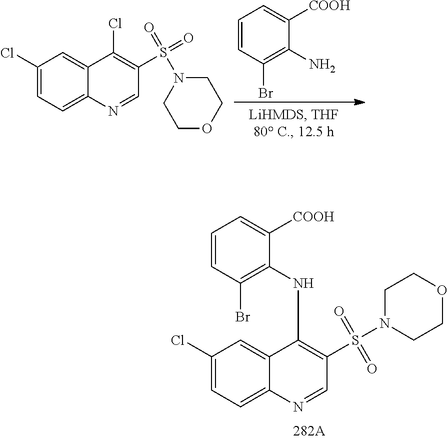

To a solution of 2-amino-3-bromo-benzoic acid (31.11 mg, 144.00 umol, 1 eq) in THF (1 mL) was added dropwise LiHMDS (1 M, 432.01 uL, 3 eq), the mixture was stirred at 20° C. for 30 minutes, then added 4-[(4,6-dichloro-3-quinolyl)sulfonyl]morpholine (50 mg, 144.00 umol, 1 eq), the mixture was purged with N₂, the reaction was stirred at 80° C. for 12 h. LCMS showed starting material was consumed completely and the MS of desired product was detected The reaction was concentrated in vacuum. The crude product was purified by prep-HPLC (column: Phenomenex Luna 80*30 mm*3 um; mobile phase: [water (0.04% HCl)-ACN]; B %: 20%-50%, 8 min). Compound 3-bromo-2-[(6-chloro-3-morpholinosulfonyl-4-quinolyl)amino]benzoic acid (24.6 mg, 42.70 umol, 29.65% yield, 97.77% purity, HCl) was obtained as yellow solid. ¹H NMR (400 MHz, DMSO-d6) δ ppm 8.56 (br s, 1H), 7.89-7.95 (m, 1H), 7.85 (dd, J=7.95, 1.34 Hz, 1H), 7.67-7.77 (m, 2H), 7.05-7.20 (m, 2H), 3.57-3.61 (m, 4H), 3.18-3.27 (m, 4H), MS (M+H)⁺=527.9

### Example 85—Synthesis of 283A

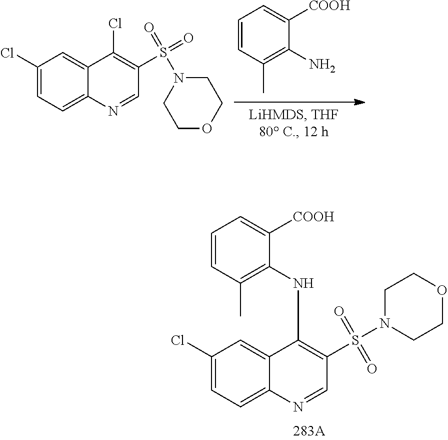

To a solution of 2-amino-3-methyl-benzoic acid (21.77 mg, 144.00 umol, 1 eq) in THF (1.5 mL) was added dropwise LiHMDS (1 M, 216.00 uL, 1.5 eq) and 4-[(4,6-dichloro-3-quinolyl)sulfonyl]morpholine (50 mg, 144.00 umol, 1 eq), the mixture was purged with N₂ for 3 times, the reaction was stirred at 80° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water (HCl)-ACN]; B %: 15%-35%,8 min) Compound 2-[(6-chloro-3-morpholinosulfonyl-4-quinolyl)amino]-3-methyl-benzoic acid (14.5 mg, 28.41 umol, 19.73% yield, 97.64% purity, HCl) was obtained as a yellow solid. ¹H NMR (400 MHz, DMSO-d6) δ ppm 8.83 (br s, 1H), 7.96 (br s, 1H), 7.85 (br d, J=7.46 Hz, 2H), 7.53-7.63 (m, 1H), 7.40 (br s, 1H), 7.01 (d, J=2.20 Hz, 1H), 3.64 (t, J=4.58 Hz, 4H), 3.18-3.31 (m, 4H), 2.03 (d, J=1.59 Hz, 3H). MS (M+H)⁺=462.0

### Example 86—Synthesis of Compound 284A

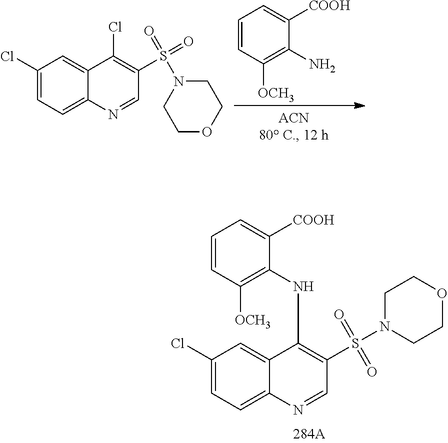

A solution of 4-[(4,6-dichloro-3-quinolyl)sulfonyl]morpholine (40 mg, 115.20 umol, 1 eq) and 2-amino-3-methoxy-benzoic acid (19.26 mg, 115.20 umol, 1 eq) in ACN (1.5 mL) was stirred at 80° C. for 12 h. LC-MS showed starting material was consumed completely and the Ms of desired product was detected. The reaction mixture was filtered, and filter cake was concentrate in vacuum. The residue was purified by prep-HPLC (column: Phenomenex Luna C18 150*30 mm*5 um; mobile phase: [water(TFA)-ACN]; B %: 20%-60%,8 min) Compound 2-[(6-chloro-3-morpholinosulfonyl-4-quinolyl)amino]-3-methoxy-benzoic acid (20.7 mg, 34.86 umol, 30.26% yield,99.68% purity, TFA) was obtained as a yellow solid. ¹H NMR (400 MHz, DMSO-d6) δ ppm 9.99-10.32 (m, 1H), 8.93 (s, 1H), 8.01 (d, J=9.01 Hz, 1H), 7.84 (dd, J=8.94, 2.31 Hz, 1H), 7.64 (dd, J=7.63, 1.63 Hz, 1H), 7.20-7.45 (m, 3H), 3.60-3.55 (m, 4H), 3.25-3.10 (m, 3H), 3.09-3.06 (m, 4H). MS (M+H)⁺=478.0

### Example 87—Synthesis of 285A

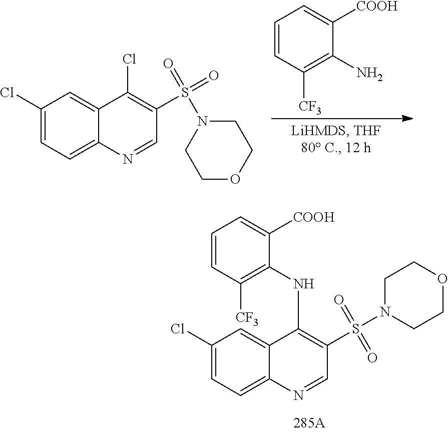

To a solution of 4-[(4,6-dichloro-3-quinolyl)sulfonyl]morpholine (50 mg, 144.00 umol, 1 eq) in THF (0.6 mL) was added 2-amino-3-(trifluoromethyl)benzoic acid (29.54 mg, 144.00 umol, 1 eq) and LiHMDS (1 M, 432.01 uL, 3 eq), the mixture was purged with N₂, the reaction was stirred at 80° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 35%-65%,8 min). Compound 2-[(6-chloro-3-morpholinosulfonyl-4-quinolyl)amino]-3-(trifluoromethyl)benzoic acid (3.2 mg, 5.54 umol, 3.85% yield, 95.69% purity, HCl) was obtained as a yellow solid. ¹H NMR (400 MHz, DMSO-d6) δ ppm 8.28 (br s, 1H), 8.05 (d, J=7.21 Hz, 1H), 7.91 (br d, J=7.34 Hz, 1H), 7.48-7.65 (m, 2H), 7.15-6.97 (m, 1H), 6.97 (d, J=1.83 Hz, 1H), 3.49-3.57 (m, 4H), 3.14-3.22 (m, 4H), MS (M+H)⁺=516.0

### Example 88—Synthesis of Compound 289A

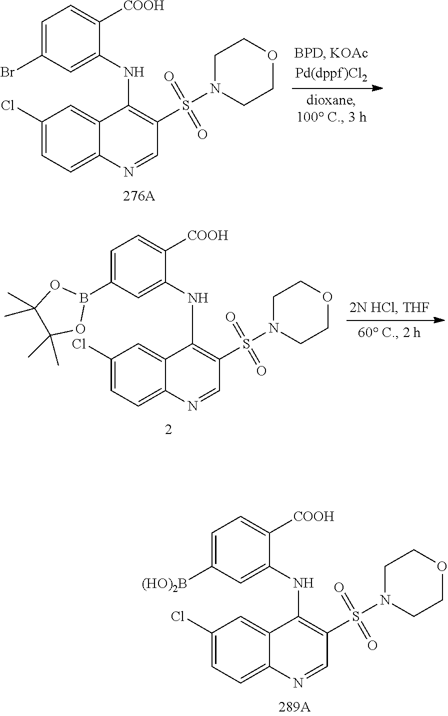 [SMILES: O=C(O)c1ccc(Br)cc1Nc1c(S(=O)(=O)N2CCOCC2)cnc2ccc(Cl)cc12 ; CC1(C)OB(c2ccc(C(=O)O)c(Nc3c(S(=O)(=O)N4CCOCC4)cnc4ccc(Cl)cc34)c2)OC1(C)C ; O=C(O)c1ccc(B(O)O)cc1Nc1c(S(=O)(=O)N2CCOCC2)cnc2ccc(Cl)cc12]

Step 1. Synthesis of 2-[(6-chloro-3-morpholinosulfonyl-4-quinolyl)amino]-4-(4,4,5,5-tetramethyl-1,3,2-dioxaborolan-2-yl)benzoic acid (2): To a stirred solution of 4-bromo-2-[(6-chloro-3-morpholinosulfonyl-4-quinolyl)amino]benzoic acid (150 mg, 284.74 umol, 1 eq) in dioxane (5 mL) was added AcOK (83.84 mg, 854.23 umol, 3 eq), BPD (86.77 mg, 341.69 umol, 1.2 eq) and Pd(dppf)Cl₂·CH₂Cl₂ (23.25 mg, 28.47 umol, 0.1 eq), the mixture was purged with Ar for 3 times, and the reaction was stirred at 100° C. for 3 h. TLC (Petroleum ether/Ethyl acetate=3:1, R_{f}=0.47) showed starting material was consumed completely and the new spot was formed. The reaction mixture was cooled to room temperature and quenched by water (10 mL), extracted with ethyl acetate (10 mL*2). The combined organics were washed with brine (5 mL), dried over Na₂SO₄, filtered and concentrated under reduced pressure to give a residue. The residue was purified by flash column (ISCO 10 g silica, 30-60% ethyl acetate in petroleum ether, gradient over 20 min). Compound 2-[(6-chloro-3-morpholinosulfonyl-4-quinolyl)amino]-4-(4,4,5,5-tetramethyl-1,3,2-dioxaborolan-2-yl)benzoic acid (100 mg, 174.26 umol, 61.20% yield) was obtained as a yellow solid.

Step 2. Synthesis of 4-borono-2-[(6-chloro-3-morpholinosulfonyl-4-quinolyl)amino]benzoic acid (289A): A solution of 2-[(6-chloro-3-morpholinosulfonyl-4-quinolyl)amino]-4-(4,4,5,5-tetramethyl-1,3,2-dioxaborolan-2-yl)benzoic acid (100 mg, 174.26 umol, 1 eq) in HCl (2 M, 87.13 uL, 1 eq) and THF (4 mL) was stirred at 60° C. for 2 h. LCMS showed the starting material was consumed completely and desired MS was detected. The reaction mixture was filtered, and filtrate was purified by prep-HPLC (column: Phenomenex Luna 80*30 mm*3 um; mobile phase: [water(0.04% HCl)-ACN]; B %: 10%-40%,8 min. Compound 4-borono-2-[(6-chloro-3-morpholinosulfonyl-4-quinolyl)amino]benzoic acid (9.40 mg, 17.02 umol, 9.77% yield, 95.64% purity, HCl) was obtained as a yellow solid. ¹H NMR (400 MHz, DMSO-d6) δ=10.31 (br s, 1H), 9.10 (s, 1H), 8.12 (d, J=9.0 Hz, 1H), 7.95 (d, J=7.8 Hz, 1H), 7.89 (dd, J=2.4, 9.0 Hz, 1H), 7.55 (d, J=2.3 Hz, 1H), 7.48 (d, J=8.0 Hz, 1H), 7.11 (s, 1H), 3.49-3.43 (m, 2H), 3.37-3.28 (m, 2H), 3.15-2.96 (m, 4H). MS (M+H)⁺=492.1.

### Example 89—Synthesis of Compound 290A

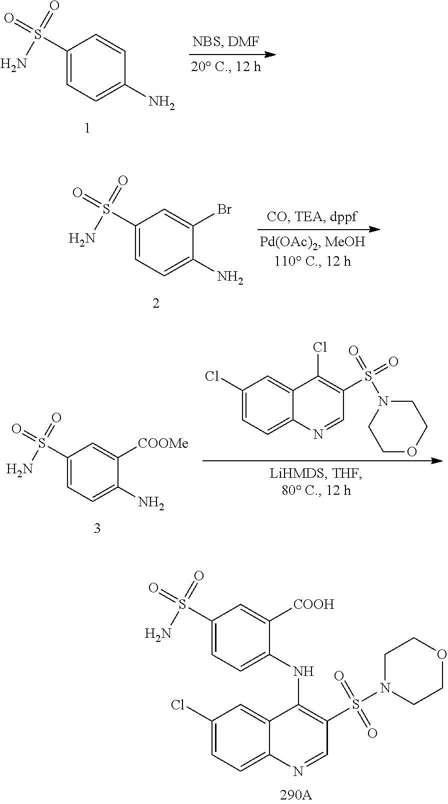 [SMILES: Nc1ccc(S(N)(=O)=O)cc1 ; Nc1ccc(S(N)(=O)=O)cc1Br ; COC(=O)c1cc(S(N)(=O)=O)ccc1N ; O=S(=O)(c1cnc2ccc(Cl)cc2c1Cl)N1CCOCC1 ; NS(=O)(=O)c1ccc(Nc2c(S(=O)(=O)N3CCOCC3)cnc3ccc(Cl)cc23)c(C(=O)O)c1]

Step 1. Synthesis of methyl 4-amino-3-bromo-benzenesulfonamide (2): To a solution of 4-aminobenzenesulfonamide (4 g, 23.23 mmol, 4.00 mL, 1 eq) in DMF (30 mL) was added NBS (3.72 g, 20.91 mmol, 0.9 eq), the mixture was stirred at 20° C. for 12 h. TLC (Petroleum ether/Ethyl acetate=3:1, R_{f}=0.66) showed starting material was consumed completely and the new spot was formed. The reaction mixture was cooled to room temperature and quenched by water (15 mL), extracted with ethyl acetate (20 mL*2). The combined organics were washed with brine (15 mL), dried over Na₂SO₄, filtered and concentrated under reduced pressure to give a residue. The residue was purified by flash column (ISCO 40 g silica, 10-40% ethyl acetate in petroleum ether, gradient over 20 min). Compound 4-amino-3-bromo-benzenesulfonamide (2.8 g, 11.15 mmol, 48.01% yield) was obtained as a yellow solid. ¹H NMR (400 MHz, DMSO-d6) δ ppm 7.75 (d, J=2.20 Hz, 1H) 7.48 (dd, J=8.56, 2.08 Hz, 1H) 7.08 (s, 2H) 6.83 (d, J=8.56 Hz, 1H) 5.88-6.16 (m, 2H).

Step 2. Synthesis of methyl 2-amino-5-sulfamoyl-benzoate (3): To a stirred solution of 4-amino-3-bromo-benzenesulfonamide (0.5 g, 1.99 mmol, 1 eq) in MeOH (10 mL) was added TEA (201.49 mg, 1.99 mmol, 277.15 uL, 1 eq), Pd(OAc)₂ (89.41 mg, 398.25 umol, 0.2 eq) and DPPF (220.78 mg, 398.25 umol, 0.2 eq) the mixture was purged with CO for 3 times, and stirred at 80° C. for 12 h under CO (15 psi). LCMS showed the starting material was consumed completely and desired product was detected. The reaction mixture was poured into water (100 mL). The aqueous phase was extracted with ethyl acetate (100 mL*2). The combined organic phase was dried with anhydrous Na₂SO₄, filtered and concentrated in vacuum. The crude product was purified by flash column (ISCO 40 g silica, 30-40% ethyl acetate in petroleum ether, gradient over 20 min). Based on TLC (Petroleum ether:Ethyl acetate=1/1, R_{f}=0.74). Compound methyl 2-amino-5-sulfamoyl-benzoate (400 mg, 1.74 mmol, 87.25% yield) was obtained as a yellow solid. MS (M+H)⁺=231.2.

Step 3. Synthesis of 2-[(6-chloro-3-morpholinosulfonyl-4-quinolyl)amino]-5-sulfamoyl-benzoic acid (290A): To a stirred solution of methyl 2-amino-5-sulfamoyl-benzoate (250 mg, 1.09 mmol, 1 eq) in THF (10 mL) was added LiHMDS (1 M, 3.26 mL, 3 eq) at 25° C., and stirred at 25° C. for 0.5 h. Then 4-[(4,6-dichloro-3-quinolyl)sulfonyl]morpholine (37 7.01 mg, 1.09 mmol, 1 eq) was added, and the mixture was heated to 80° C. for 12 h. LCMS showed the starting material was consumed completely and 30% desired MS was detected. The reaction mixture was added dropwise sat. NH₄Cl (5 mL). Then the mixture was concentrate in vacuum. The residue was purified by prep-HPLC (column: Phenomenex luna C18 25 0*50 mm*10 um; mobile phase: [water(HCl)-ACN]; B %: 20%-50%,10 min). Afford curde product 10 mg. The crude product was purified by prep-HPLC (column: Phenomenex luna C18 80*40 mm*3 um; mobile phase: [water(HCl)-ACN]; B %: 25%-47%,7 min). Compound 2-[(6-chloro-3-morpholinosulfonyl-4-quinolyl)amino]-5-sulfamoyl-benzoic acid (3.80 mg, 6.74 umol, 6.21e-1% yield, 100% purity, HCl) was obtained as a yellow solid. ¹H NMR (400 MHz, DMSO-d6) δ=10.67 (br s, 1H), 9.17 (s, 1H), 8.44 (d, J=2.3 Hz, 1H), 8.20 (d, J=9.0 Hz, 1H), 7.96 (dd, J=2.2, 9.1 Hz, 1H), 7.72 (d, J=2.1 Hz, 1H), 7.67 (dd, J=2.3, 8.8 Hz, 1H), 7.35 (br s, 2H), 6.70 (d, J=8.8 Hz, 1H), 3.54-3.45 (m, 2H), 3.40-3.32 (m, 2H), 3.03 (br t, J=4.4 Hz, 4H). MS (M+H)⁺=527.1

### Example 90—Synthesis of Compound 292A

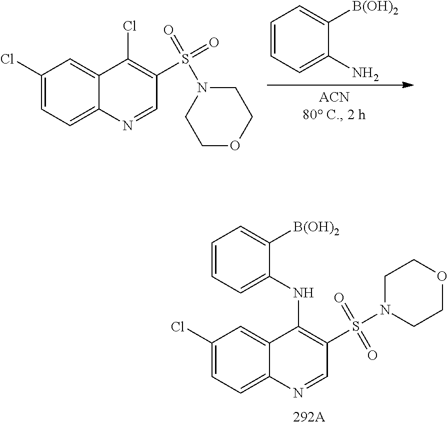 [SMILES: O=S(=O)(c1cnc2ccc(Cl)cc2c1Cl)N1CCOCC1 ; Nc1ccccc1B(O)O ; O=S(=O)(c1cnc2ccc(Cl)cc2c1Nc1ccccc1B(O)O)N1CCOCC1]

A solution of 4-[(4,6-dichloro-3-quinolyl)sulfonyl]morpholine (40 mg, 115.20 umol, 1 eq) and (2-aminophenyl)boronic acid (15.78 mg, 115.20 umol, 1 eq) in ACN (1.5 mL) was stirred at 80° C. for 12 h. LC-MS showed starting material was consumed completely and the Ms of desired product was detected. The reaction mixture was filtered, and filter cake was concentrate in vacuum. The residue was purified by prep-HPLC (column: Waters Xbridge BEH C18 100*30 mm*10 um; mobile phase: [water(NH₄HCO₃)-ACN]; B %: 30%-60%,10 min). Compound [2-[(6-chloro-3-morpholinosulfonyl-4-quinolyl)amino]phenyl]boronic acid (19 mg, 42.44 umol, 36.84% yield) was obtained as a yellow solid. ¹H NMR (400 MHz, DMSO-d6) δ ppm 8.91 (s, 1H), 7.99 (br d, J=8.68 Hz, 1H), 7.77 (br d, J=7.82 Hz, 2H), 7.51 (d, J=1.83 Hz, 1H), 7.14-7.21 (m, 1H), 6.99-7.06 (m, 1H), 6.57 (d, J=8.19 Hz, 1H), 3.40-3.54 (m, 4H), 3.07-3.16 (m, 2H), 2.96-3.07 (m, 2H). MS (M+H)⁺=448.0

### Example 91—Synthesis of 293A

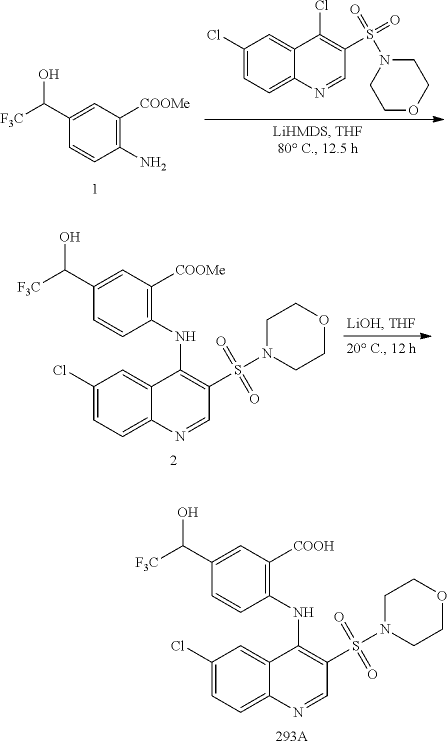 [SMILES: COC(=O)c1cc(C(O)C(F)(F)F)ccc1N ; O=S(=O)(c1cnc2ccc(Cl)cc2c1Cl)N1CCOCC1 ; COC(=O)c1cc(C(O)C(F)(F)F)ccc1Nc1c(S(=O)(=O)N2CCOCC2)cnc2ccc(Cl)cc12 ; O=C(O)c1cc(C(O)C(F)(F)F)ccc1Nc1c(S(=O)(=O)N2CCOCC2)cnc2ccc(Cl)cc12]

Step 1. Synthesis of methyl 2-[(6-chloro-3-morpholinosulfonyl-4-quinolyl)amino]-5-(2,2,2-trifluoro-1-hydroxy-ethyl)benzoate (2): To a solution of 4-[(4,6-dichloro-3-quinolyl) sulfonyl]morpholine (278.68 mg, 802.61 umol, 1 eq) in THF (3 mL) was added LiHMDS (1 M, 2.41 mL, 3 eq). The mixture was purged with N₂ for 3 times, the mixture was stirred at 20° C. for 30 minutes. Then methyl 2-amino-5-(2,2,2-trifluoro-1-hydroxy-ethyl)benzoate (200.00 mg, 802.61 umol, 1 eq) was added, the solution was purged with N₂, the reaction was stirred at 80° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 30%-60%,8 min) Compound methyl 2-[(6-chloro-3-morpholinosulfonyl-4-quinolyl)amino]-5-(2,2,2-trifluoro-1-hydroxy-ethyl)benzoate (25 mg, 44.65 umol, 5.56% yield) was obtained as a yellow solid. MS (M+H)⁺=560.2

Step 2. Synthesis of methyl 2 2-[(6-chloro-3-morpholinosulfonyl-4-quinolyl)amino]-5-(2,2,2-trifluoro-1-hydroxy-ethyl)benzoic acid (293A): To a solution of methyl 2-[(6-chloro-3-morpholinosulfonyl-4-quinolyl)amino]-5-(2,2,2-trifluoro-1-hydroxy-ethyl)benzoate (10 mg, 17.86 umol, 1 eq) in THF (0.6 mL) was added LiOH (2 M, 8.93 uL, 1 eq) the mixture was stirred at 20° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 25%-65%,8 min) Compound 2-[(6-chloro-3-morpholinosulfonyl-4-quinolyl)amino]-5-(2,2,2-trifluoro-1-hydroxy-ethyl)benzoic acid (2.3 mg, 3.95 umol, 22.11% yield, 100% purity, HCl) was obtained as a yellow solid. ¹H NMR (400 MHz, DMSO-d6) δ ppm 10.43 (br s, 1H), 9.11 (s, 1H), 8.06-8.23 (m, 2H), 7.91 (br d, J=8.80 Hz, 1H), 7.60 (br s, 1H), 7.42 (br d, J=8.68 Hz, 1H), 6.68 (br d, J=8.56 Hz, 1H), 5.05-5.32 (m, 1H), 3.35 (br s, 4H), 2.94-3.13 (m, 4H). MS (M+H)⁺=546.0

### Example 92—Synthesis of 295A

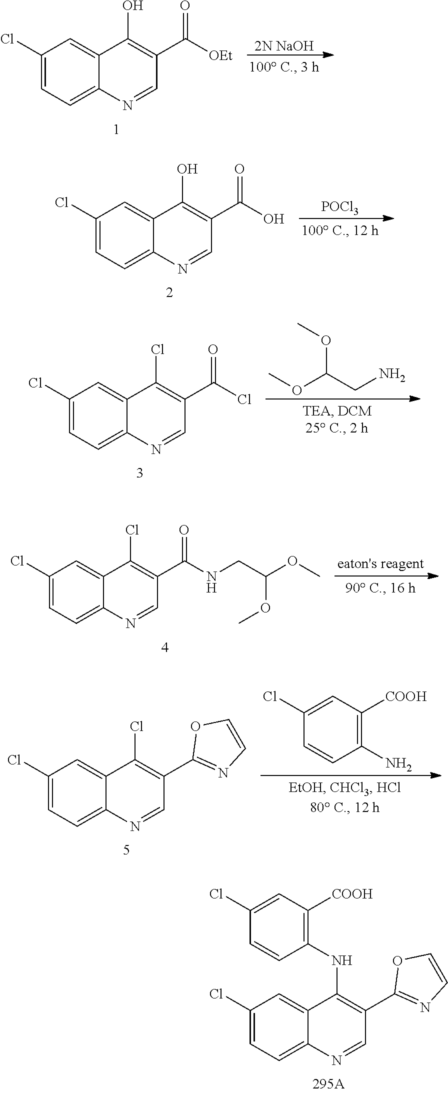

Step 1. Synthesis of 6-chloro-4-hydroxy-quinoline-3-carboxylic acid (2): A suspension of ethyl 6-chloro-4-hydroxy-quinoline-3-carboxylate (1.7 g, 6.76 mmol, 1 eq) in NaOH (17 mL) was stirred for 3 h at 100° C. LCMS showed starting material was consumed completely and the MS of desired product was detected. The mixture was acidified with 2 N HCl to pH=3. The resulting precipitate was collected by filtration. Compound 6-chloro-4-hydroxy-quinoline-3-carboxylic acid (1.2 g, 5.37 mmol, 79.44% yield) was obtained as a white solid. MS (M+H)⁺=224.2.

Step 2. Synthesis of 4,6-dichloroquinoline-3-carbonyl chloride (3): A solution of 6-chloro-4-hydroxy-quinoline-3-carboxylic acid (1.2 g, 5.37 mmol, 1 eq) in POCl₃ (15 mL), the mixture was purged with N₂ for 3 times. The reaction was stirred at 100° C. for 12 h. LCMS showed starting material was consumed completely and desired product was detected. The reaction mixture was concentrated in vacuum. Compound 4,6-dichloroquinoline-3-carbonyl chloride (2 g, crude) was obtained as a white solid.

Step 3. Synthesis of 4,6-dichloro-N-(2,2-dimethoxyethyl)quinoline-3-carboxamide (4): To a stirred solution of 4,6-dichloroquinoline-3-carbonyl chloride (2.5 g, 9.60 mmol, 1 eq) and TEA (2.91 g, 28.79 mmol, 4.01 mL, 3 eq) in DCM (30 mL) was added 2,2-dimethoxyethanamine (1.01 g, 9.60 mmol, 1.05 mL, 1 eq) at 0° C., then the mixture was stirred at 25° C. for 2 h. LCMS showed the starting material was consumed completely and desired MS was detected. The reaction mixture was poured into water (100 mL). The aqueous phase was extracted with ethyl acetate (200 mL*2). The combined organic phase was dried with anhydrous Na₂SO₄, filtered and concentrated in vacuum. The crude product was purified by flash column (ISCO 40 g silica, 50-60% ethyl acetate in petroleum ether, gradient over 20 min). Based on TLC (Petroleum ether:Ethyl acetate=1/1, R_{f}=0.67). Compound 4,6-dichloro-N-(2,2-dimethoxyethyl)quinoline-3-carboxamide (1.2 g, 3.65 mmol, 37.99% yield) was obtained as a white solid. MS (M+H)⁺=329.2. ¹H NMR (400 MHz, CHLOROFORM-d) δ=9.00 (s, 1H), 8.27 (d, J=2.2 Hz, 1H), 8.07 (d, J=8.9 Hz, 1H), 7.76 (dd, J=2.3, 9.0 Hz, 1H), 6.56 (br s, 1H), 4.58 (t, J=5.2 Hz, 1H), 3.70 (t, J=5.5 Hz, 2H), 3.47 (s, 6H).

Step 4. Synthesis of 2-(4,6-dichloro-3-quinolyl)oxazole (5): A solution of 4,6-dichloro-N-(2,2-dimethoxyethyl)quinoline-3-carboxamide (300 mg, 911.36 umol, 1 eq) in EATON'S REAGENT (6 mL) was stirred at 90° C. for 16 h. LCMS showed the starting material was consumed completely and desired product was detected. The reaction mixture was poured into water (4 mL). The aqueous phase was extracted with dichloromethane (4 mL*2). The combined organic phase was dried with anhydrous Na₂SO₄, filtered and concentrated in vacuum. Compound 2-(4,6-dichloro-3-quinolyl)oxazole (200 mg, crude) was obtained as a brown oil. MS (M+H)⁺=265.1.

Step 5. Synthesis of 5-chloro-2-[(6-chloro-3-oxazol-2-yl-4-quinolyl)amino]benzoic acid (295A): To a stirred solution of 2-(4,6-dichloro-3-quinolyl)oxazole (40 mg, 150.89 umol, 1 eq) in EtOH (0.8 mL) and CHCl₃ (0.2 mL) was added 2-amino-5-chloro-benzoic acid (25.89 mg, 150.89 umol, 1 eq) HCl (12 M, 1.26 uL, 0.1 eq), the mixture was stirred at 80° C. for 12 h. LCMS showed the starting material was consumed completely and desired MS was detected. The reaction mixture was concentrate in vacuum. The crude product was purified by prep-HPLC (column: Phenomenex luna C18 80*40 mm*3 um; mobile phase: [water(0.1% TFA)-ACN]; B %: 32%-52%,7 min). Compound 5-chloro-2-[(6-chloro-3-oxazol-2-yl-4-quinolyl)amino]benzoic acid (3.5 mg, 7.81 umol, 5.18% yield, 97.46% purity, HCl) was obtained as a yellow solid. ¹H NMR (400 MHz, DMSO-d6) δ=11.01 (br s, 1H), 9.37 (s, 1H), 8.30 (d, J=0.6 Hz, 1H), 8.10 (d, J=9.0 Hz, 1H), 7.93 (d, J=2.6 Hz, 1H), 7.85 (dd, J=2.3, 9.0 Hz, 1H), 7.75 (d, J=2.1 Hz, 1H), 7.50 (d, J=0.8 Hz, 1H), 7.33 (dd, J=2.6, 8.9 Hz, 1H), 6.64 (br d, J=8.9 Hz, 1H). MS (M+H)⁺=399.9.

### Example 93—Synthesis of Compound 296A

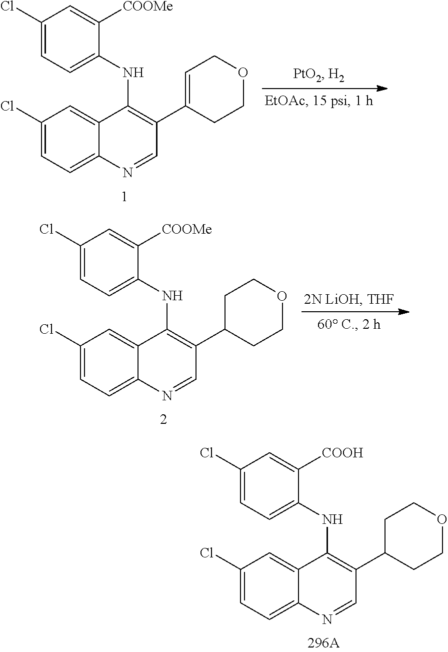 [SMILES: COC(=O)c1cc(Cl)ccc1Nc1c(C2=CCOCC2)cnc2ccc(Cl)cc12 ; COC(=O)c1cc(Cl)ccc1Nc1c(C2CCOCC2)cnc2ccc(Cl)cc12 ; O=C(O)c1cc(Cl)ccc1Nc1c(C2CCOCC2)cnc2ccc(Cl)cc12]

Step 1. Synthesis of methyl 5-chloro-2-[(6-chloro-3-tetrahydropyran-4-yl-4-quinolyl)amino]benzoate (2): To a stirred solution of methyl 5-chloro-2-[[6-chloro-3-(3,6-dihydro-2H-pyran-4-yl)-4-quinolyl]amino]benzoate (100 mg, 232.94 umol, 1 eq) in EtOAc (2 mL) was added PtO2 (52.90 mg, 232.94 umol, 1 eq) at 25° C., then the mixture was purged with H₂ for 3 times, and stirred at 25° C. for 1 h under H₂ (15 psi). LCMS showed 20% the Ms of desired product was detected. The reaction mixture was filtered, and filtrate was concentrate in vacuum. The residue was purified by prep-HPLC (column: Phenomenex Luna 80*30 mm*3 um; mobile phase: [water(0.04% HCl)-ACN]; B %: 30%-60%,8 min). Compound methyl 5-chloro-2-[(6-chloro-3-tetrahydropyran-4-yl-4-quinolyl)amino]benzoate (15 mg, 34.78 umol, 14.93% yield) was obtained as a yellow solid. MS (M+H)⁺=431.2.

Step 2. Synthesis of 5-chloro-2-[(6-chloro-3-tetrahydropyran-4-yl-4-quinolyl)amino]benzoic acid (296A): To a stirred solution of methyl 5-chloro-2-[(6-chloro-3-tetrahydropyran-4-yl-4-quinolyl)amino]benzoate (15 mg, 34.78 umol, 1 eq) in THF (0.3 mL) and MeOH (0.3 mL) was added LiOH·H₂O (2.92 mg, 69.56 umol, 2 eq) at 25° C., then the mixture was stirred at 60° C. for 2 h. LCMS showed the starting material was consumed completely and desired MS was detected. The reaction mixture was adjusted pH˜4 by adding 2N HCl. The mixture was concentrate in vacuum. The residue was purified by prep-HPLC (column: Phenomenex luna C18 80*40 mm*3 um; mobile phase: [water(0.04% HCl)-ACN]; B %: 25%-50%, 7 min). Compound 5-chloro-2-[(6-chloro-3-tetrahydropyran-4-yl-4-quinolyl)amino]benzoic acid (10.3 mg, 22.38 umol, 64.36% yield, 98.61% purity, HCl) was obtained as a yellow solid. ¹H NMR (400 MHz, DMSO-d6+D₂O) δ=8.86 (s, 1H), 8.05 (d, J=9.0 Hz, 1H), 7.91 (d, J=2.6 Hz, 1H), 7.87 (dd, J=2.2, 9.1 Hz, 1H), 7.80 (d, J=2.1 Hz, 1H), 7.45 (dd, J=2.6, 8.9 Hz, 1H), 6.71 (d, J=8.9 Hz, 1H), 3.90 (br s, 2H), 3.37-3.14 (m, 2H). 3.02 (ddd, J=4.1, 1.1.4, 15.1 Hz, 1H), 1.97-1.82 (m, 1H), 1.79-1.56 (m, 3H). MS (M+H)⁺=417.0.

### Example 93A—Synthesis of Compound 296A

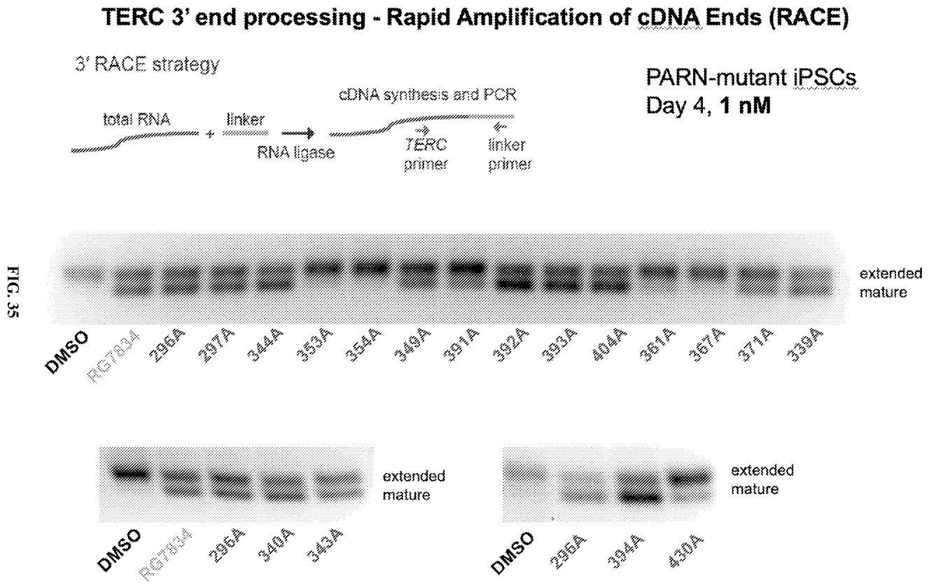

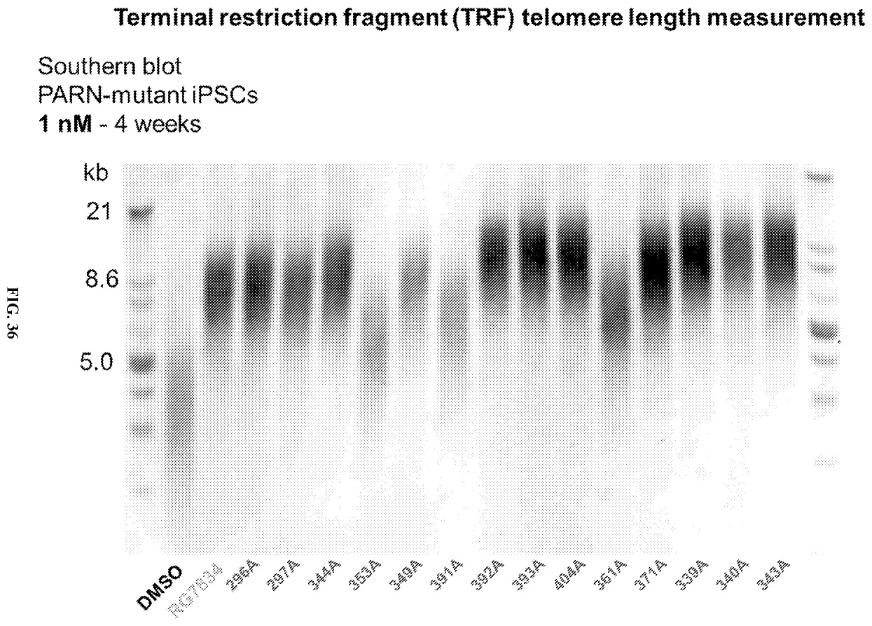

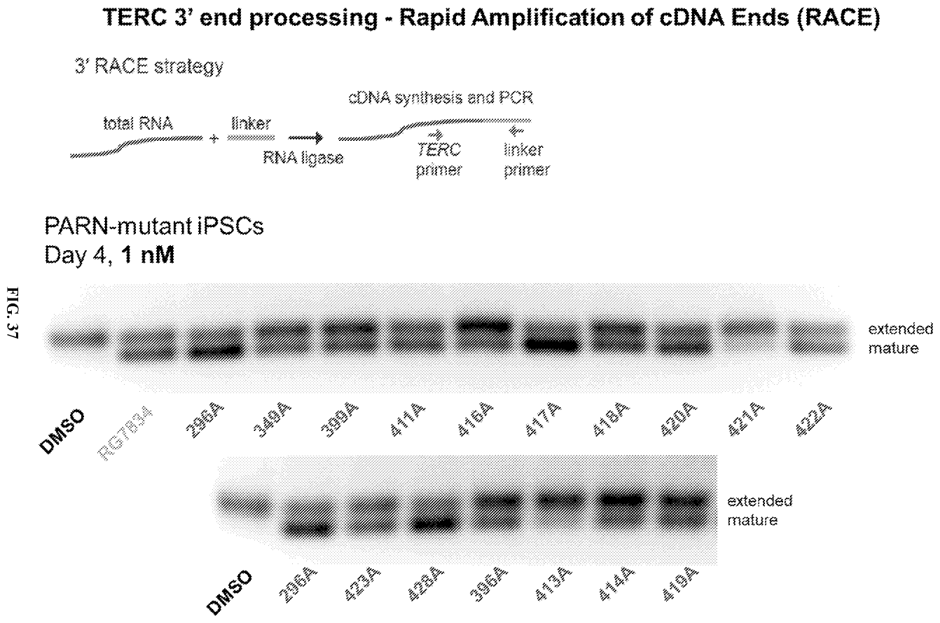

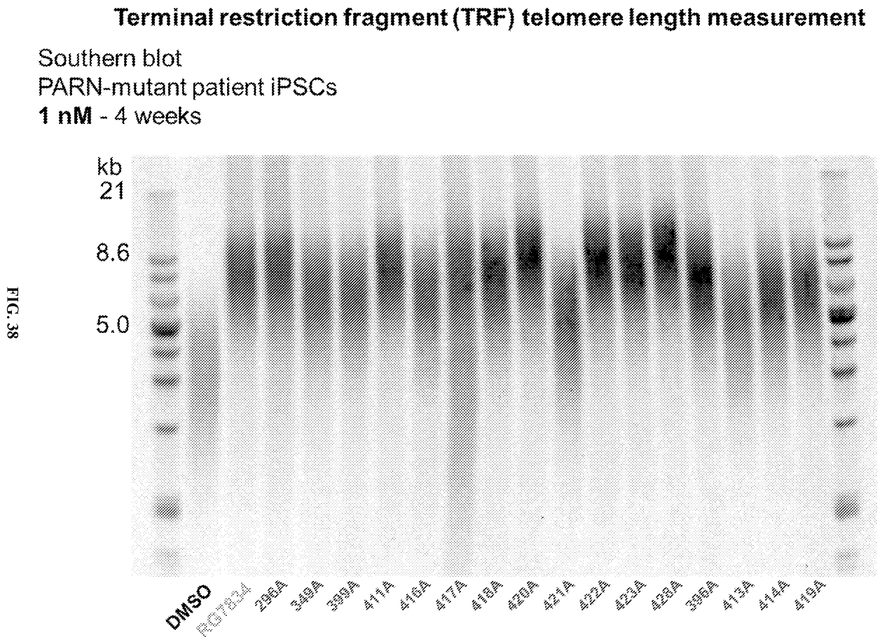

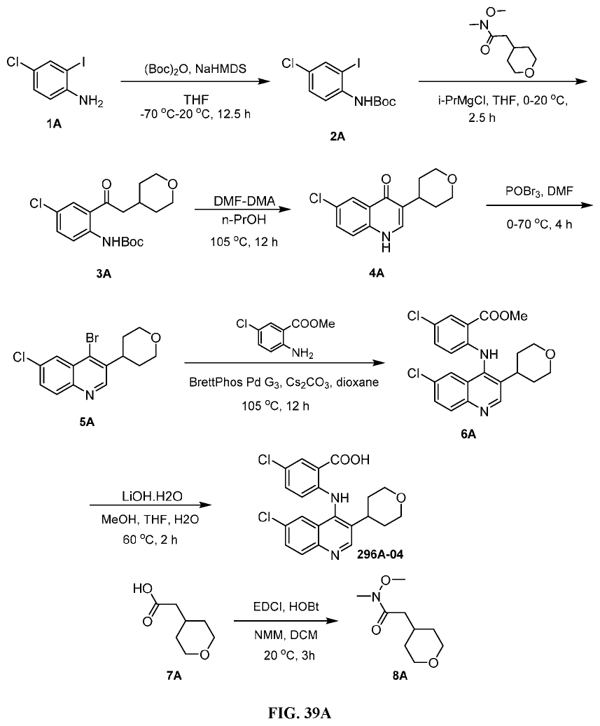

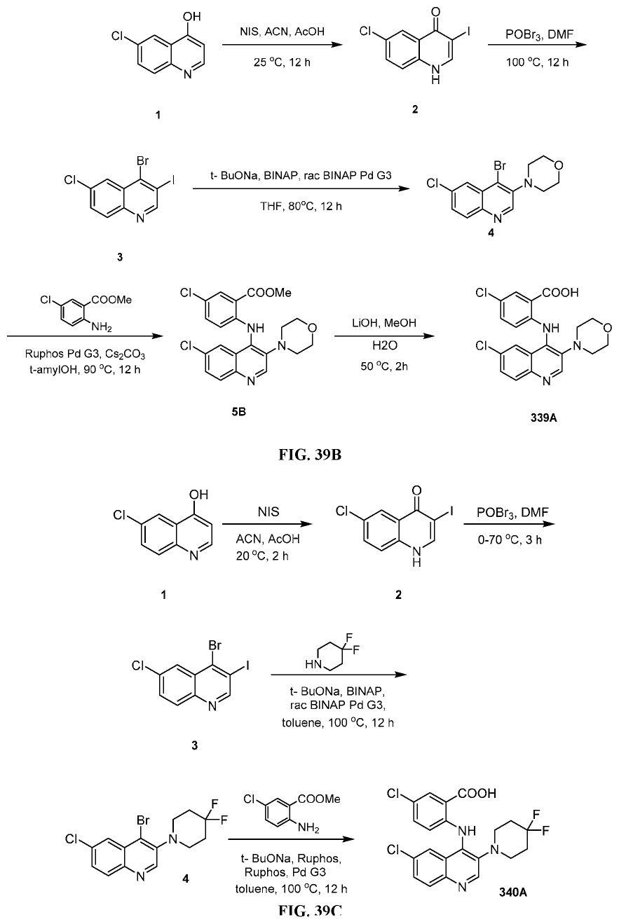

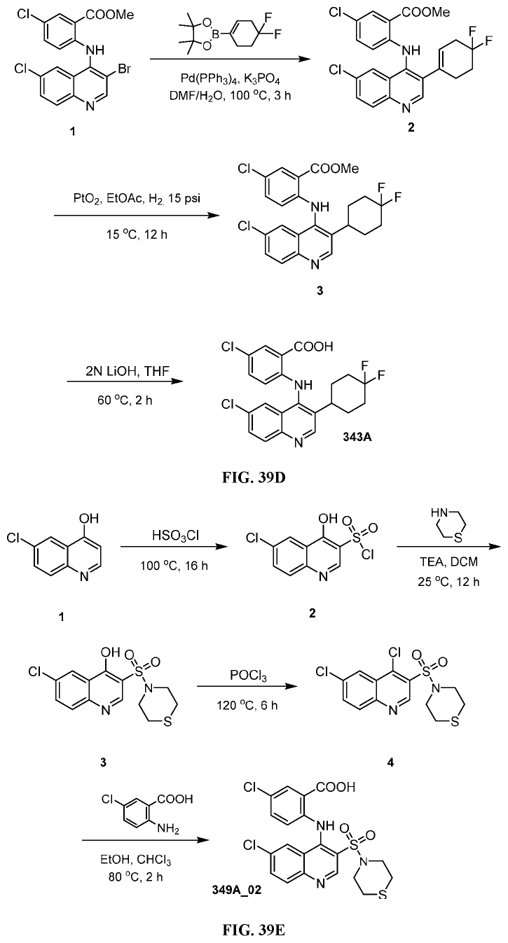

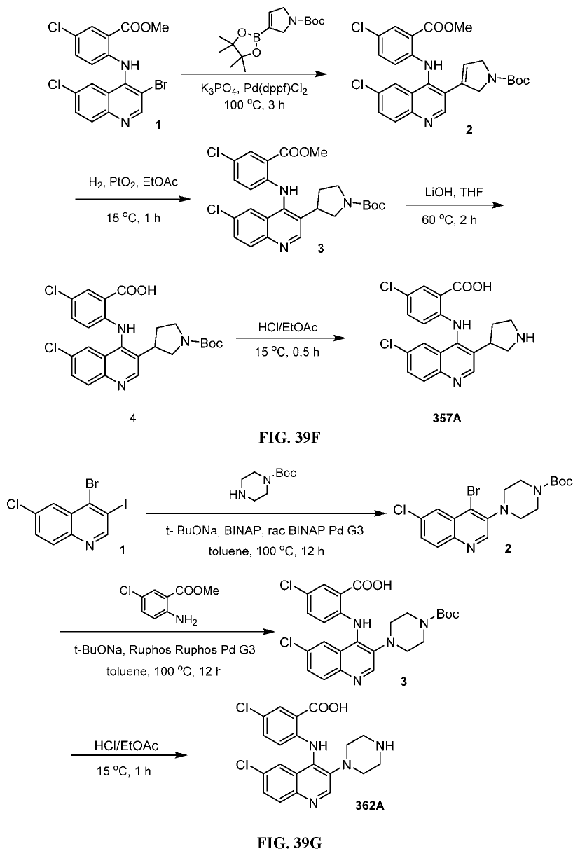

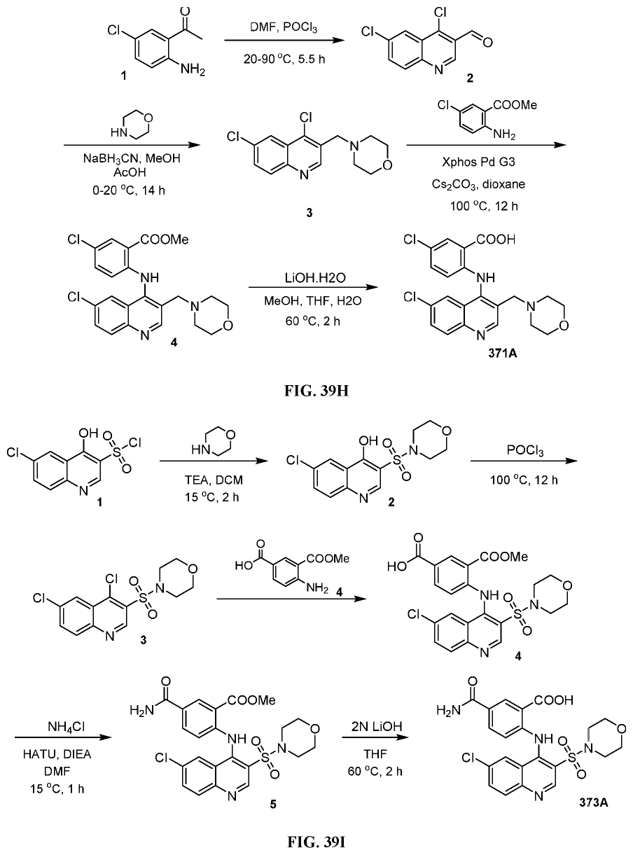

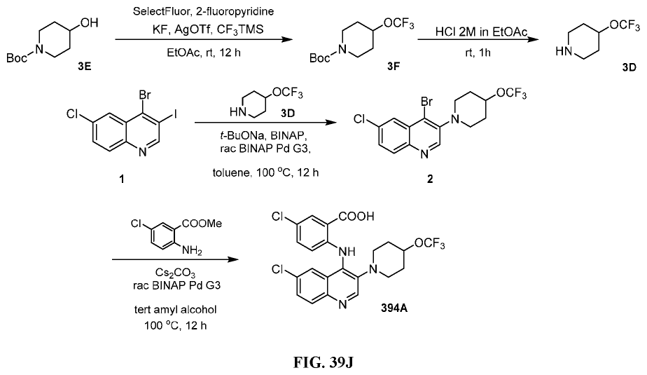

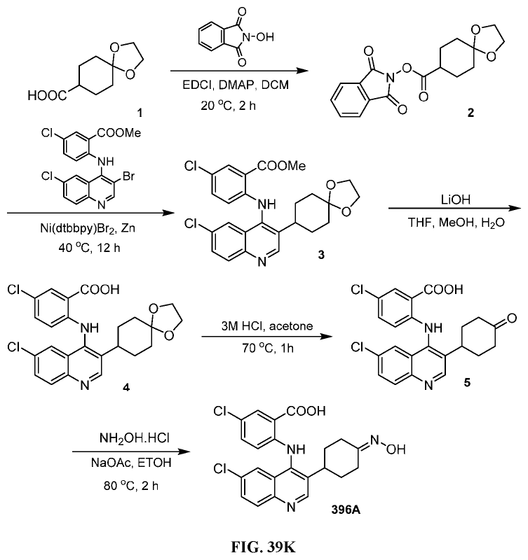

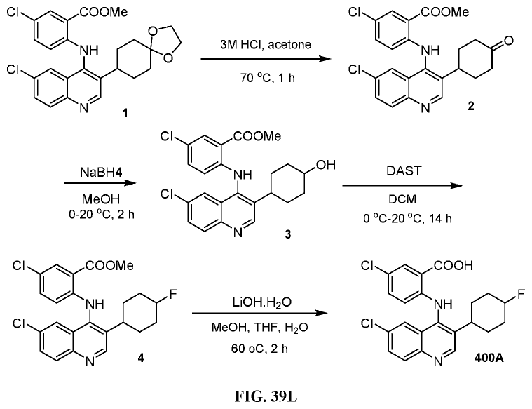

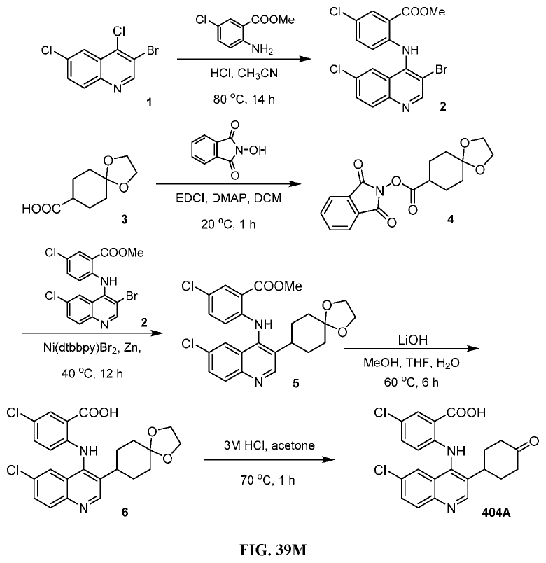

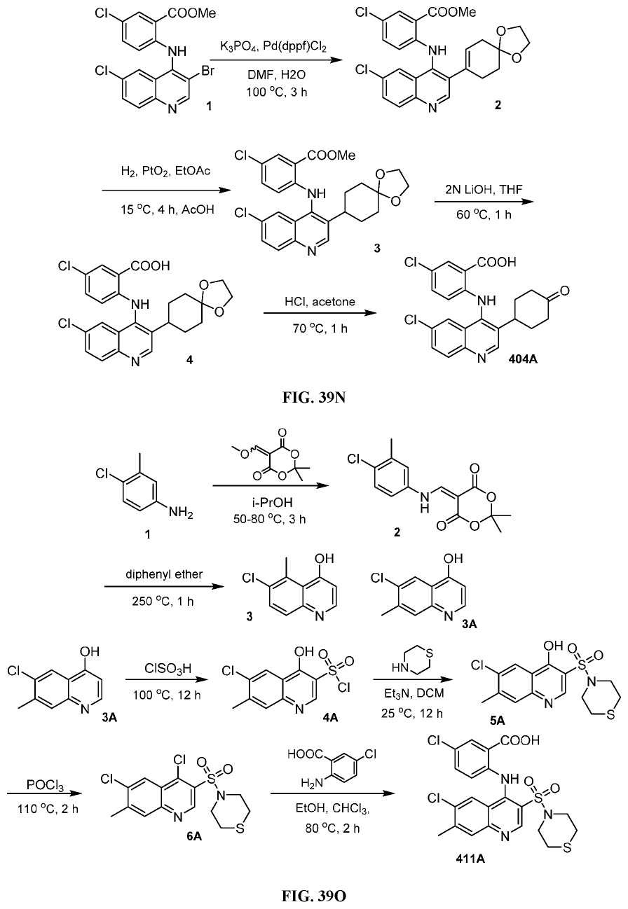

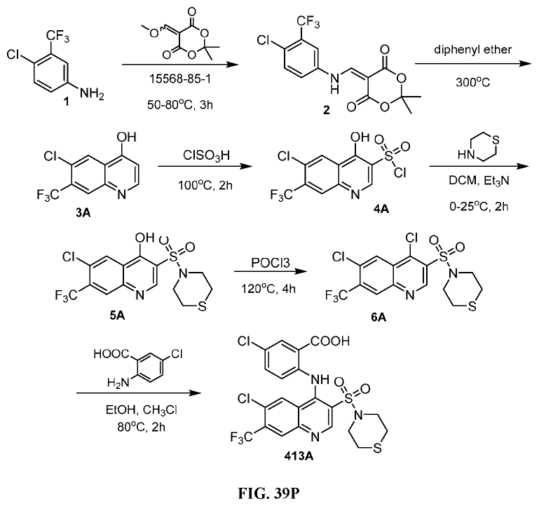

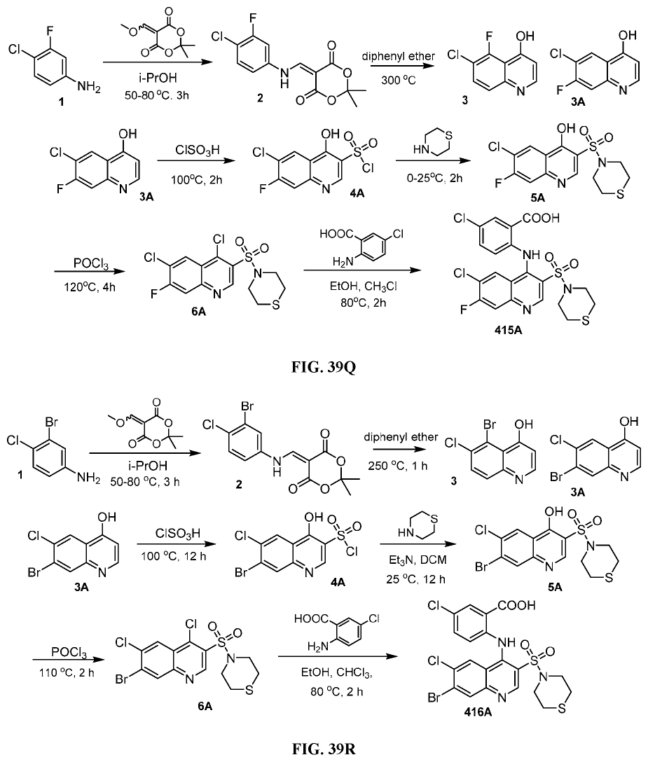

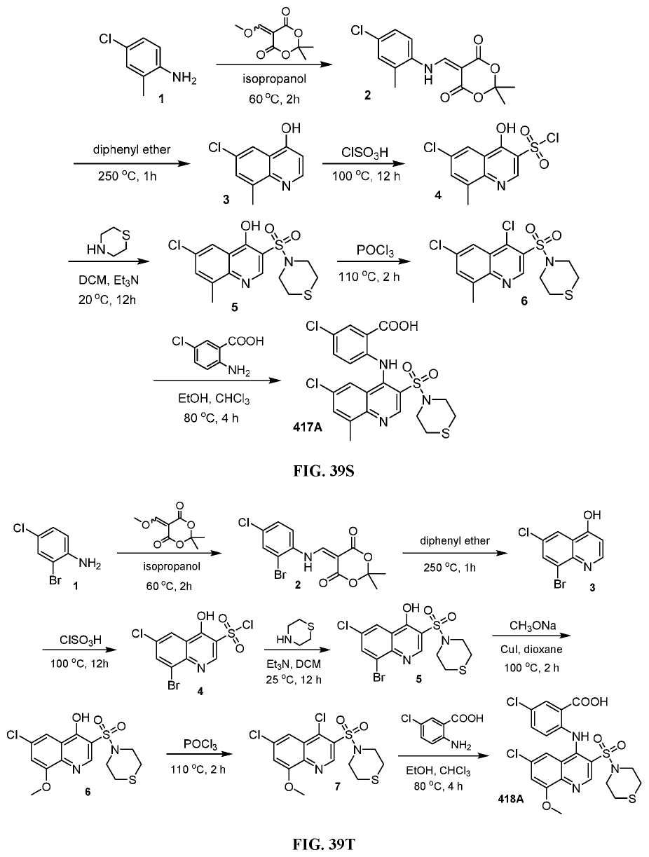

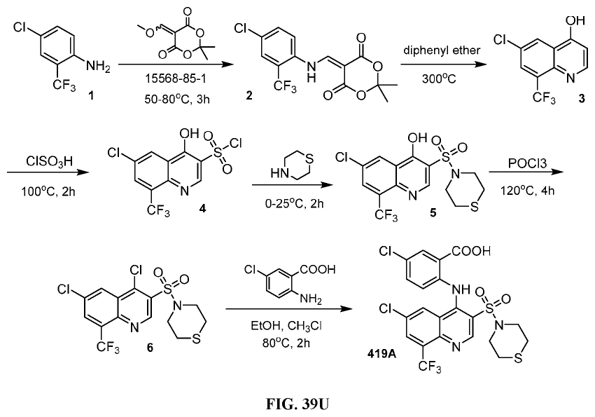

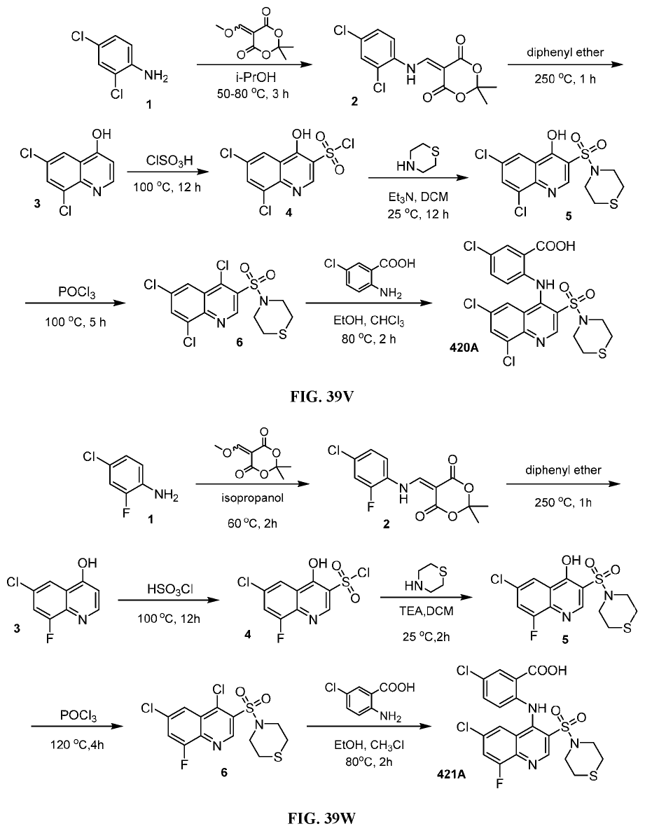

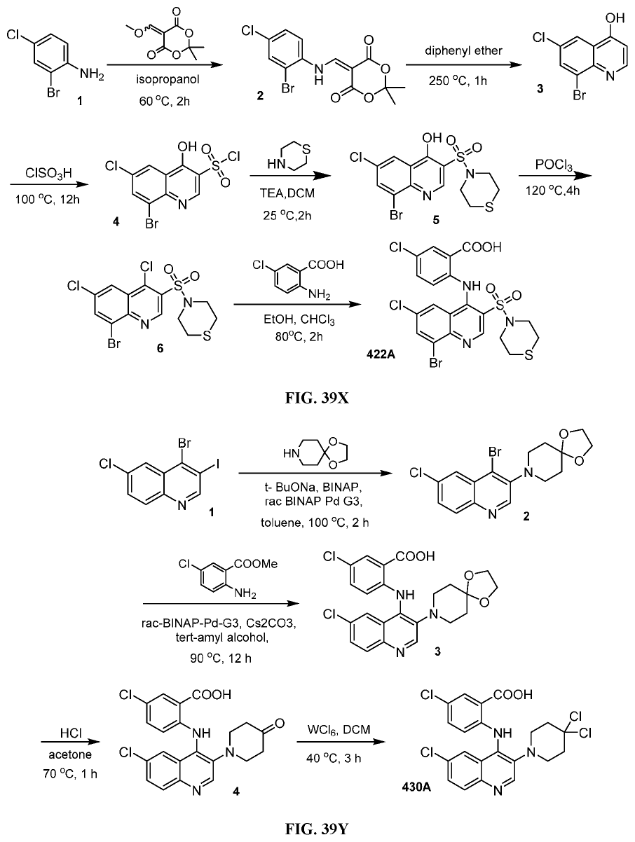

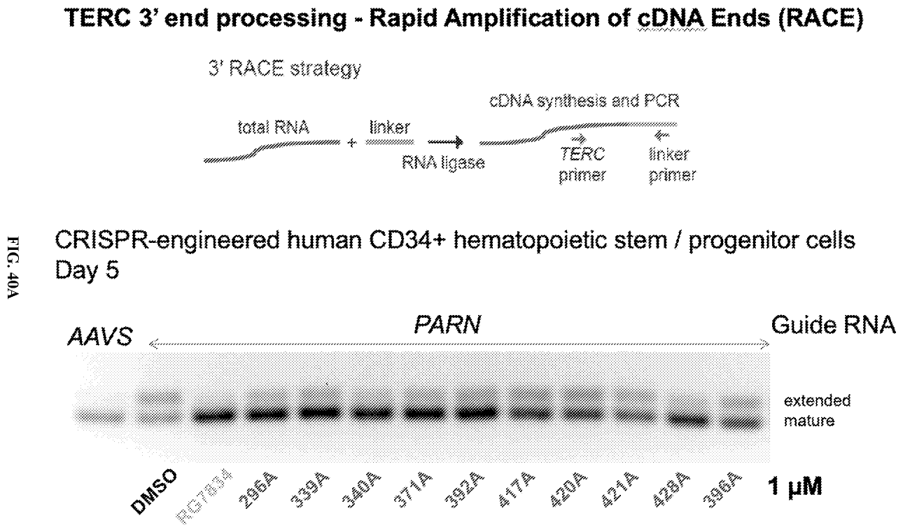

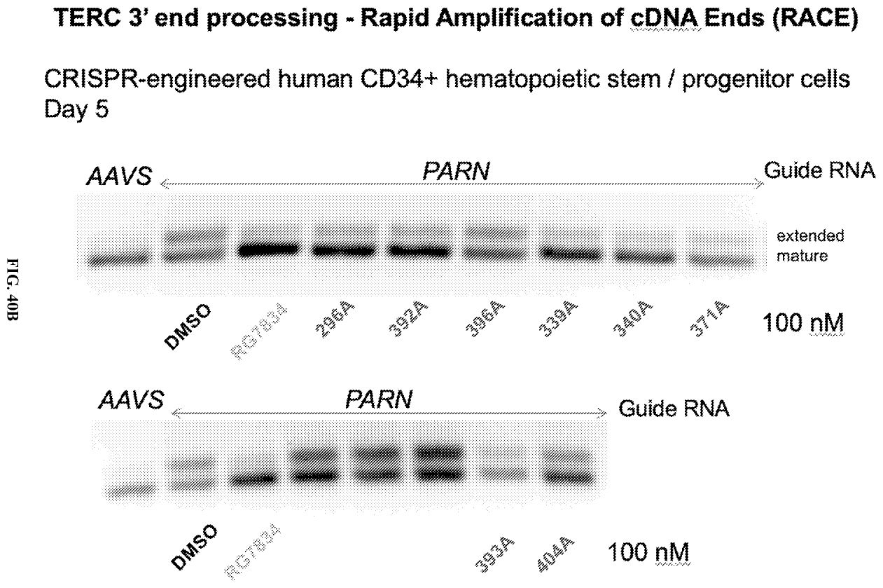

Synthetic scheme is provided in FIG. 39A

### Synthesis of N-methoxy-N-methyl-2-tetrahydropyran-4-yl-acetamide (8A)

1. To a solution of 2-tetrahydropyran-4-ylacetic acid (15 g, 104.05 mmol, 1 eq) and N-methoxymethanamine; hydrochloride (12.18 g, 124.85 mmol, 1.2 eq) in DCM (200 mL) was added HOBt (16.87 g, 124.85 mmol, 1.2 eq), EDCI (23.93 g, 124.85 mmol, 1.2 eq) and NMM (42.10 g, 416.18 mmol, 45.76 mL, 4 eq), the mixture was stirred at 20° C. for 2 h. LCMS showed the reaction was complete. 100 mL of water was added to the mixture, the mixture was extracted with DCM (50 mL*2), and the combined extracts was dried with anhydrous Na₂SO₄ and filtered. The filtrate was concentrated under reduced pressure to give a residue. The residue was purified by flash column (ISCO 20 g silica, 0-20% ethyl acetate in petroleum ether, gradient over 20 min). N-methoxy-N-methyl-2-tetrahydropyran-4-yl-acetamide (10.80 g, crude) was obtained as a brown oil. ¹H NMR (400 MHz, CHLOROFORM-d) δ 3.94-3.91 (m, 2H), 3.67 (s, 3H), 3.50-3.35 (m, 2H), 3.17 (s, 3H), 2.42-2.29 (m, 2H), 2.16-2.05 (m, 1H), 1.70-1.60 (m, 2H), 1.37-1.31 (s, 2H).

### Synthesis of tert-butyl N-(4-chloro-2-iodo-phenyl)carbamate (2A)

To a solution of 4-chloro-2-iodo-aniline (20 g, 78.91 mmol, 1 eq) in THF (300 mL) was added NaHMDS (1 M, 181.48 mL, 2.3 eq) at −70° C. for 0.5 h, then tert-butoxycarbonyl tert-butyl carbonate (17.22 g, 78.91 mmol, 18.13 mL, 1 eq) in THF (50 mL) was added drop wise to it at −70° C. and stirred at 20° C. for 12 h under N₂ atmosphere. TLC (Petroleum ether:Ethyl acetate=10:1) showed R₁(Rf=0.19) was consumed completely and a major spot (Rf=0.59) was detected. The mixture was quenched by H₂O (100 mL) at 0° C., the mixture was extracted with ethyl acetate (200 mL*2), and the combined extracts was dried with anhydrous Na₂SO₄ and filtered, the filtrate was concentrated under reduced pressure to give a residue. The residue was purified by flash column (ISCO 80 g silica, 0-10% ethyl acetate in petroleum ether, gradient over 20 min). Compound tert-butyl N-(4-chloro-2-iodo-phenyl)carbamate (21 g, 59.39 mmol, 75.27% yield) was obtained as a yellow solid.

### Synthesis of tert-butyl N-[4-chloro-2-(2-tetrahydropyran-4-ylacetyl)phenyl]carbamate (3A)

To a solution of tert-butyl N-(4-chloro-2-iodo-phenyl)carbamate (20 g, 56.56 mmol, 1 eq) in THF (200 mL) was added i-PrMgCl (2 M, 84.85 mL, 3 eq) at 0° C. for 0.5 h, then N-methoxy-N-methyl-2-tetrahydropyran-4-yl-acetamide (10.59 g, 56.56 mmol, 1 eq) in THF (110 mL) was added to the above mixture at 0° C., the mixture was stirred at 20° C. for 12 h. TLC (Petroleum ether:Ethyl acetate=1:1) showed the starting material was consumed completely and a major spot was detected. 200 mL of water was added to the mixture, the mixture was extracted with ethyl acetate (500 mL*2). The combined extracts was dried with anhydrous Na₂SO₄ and filtered, the filtrate was concentrated under reduced pressure to give a residue. The residue was purified by flash column (ISCO 80 g silica, 0-10% ethyl acetate in petroleum ether, gradient over 20 min). tert-butyl N-[4-chloro-2-(2-tetrahydropyran-4-ylacetyl)phenyl]carbamate (14.2 g, 36.12 mmol, 63.85% yield) was obtained as a yellow solid. ¹H NMR (400 MHz, CHLOROFORM-d) δ 8.49 (d, J=9.0 Hz, 1H), 7.81 (d, J=2.5 Hz, 1H), 7.47 (dd, J=2.0, 9.0 Hz, TH), 3.97 (br dd, J=3.8, 11.3 Hz, 2H), 3.46 (t, J=11.8 Hz, 2H), 2.91 (d, J=7.0 Hz, 2H), 2.30-2.18 (m, 1H), 1.69 (br dd, J=1.3, 12.8 Hz, 2H), 1.53 (s, 9H), 1.45-1.37 (m, 2H)

### Synthesis of 6-chloro-3-tetrahydropyran-4-yl-1H-quinolin-4-one (4A)

7. To a solution of tert-butyl N-[4-chloro-2-(2-tetrahydropyran-4-ylacetyl)phenyl]carbamate (14.2 g, 40.13 mmol, 1 eq) in n-PrOH (150 mL) was added DMF-DMA (23.91 g, 20.66 mmol, 26.66 mL, 5 eq), the mixture was stirred at 105° C. for 12 h. LCMS showed the reaction was complete. The mixture was concentrated under reduced pressure to give the crude product. The crude product was triturated with THF (200 ml) at 20° C. for 5 min. 6-chloro-3-tetrahydropyran-4-yl-1H-quinolin-4-one (5 g, crude) was obtained as a white solid. MS (M+H)⁺=264.3

### Synthesis of 4-bromo-6-chloro-3-tetrahydropyran-4-yl-quinoline (5A)

To a solution of 6-chloro-3-tetrahydropyran-4-yl-1H-quinolin-4-one (4 g, 15.17 mmol, 1 eq) in DMF (40 mL) was added POBr₃ (5.65 g, 19.72 mmol, 2.00 mL, 1.3 eq) in portions at 0° C. The mixture was stirred at 70° C. for 4 h. LCMS showed the starting material was consumed completely and desired MS was detected. The reaction mixture was quenched with 30 mL water and extracted with Ethyl acetate (40 mL×3). The combined organic layers were washed with brine (30 mL), dried over Na₂SO₄ and concentrated to dryness to give residue. The crude product was purified by flash column (ISCO 20 g silica, 0-40% ethyl acetate in petroleum ether, gradient over 20 min). Compound 4-bromo-6-chloro-3-tetrahydropyran-4-yl-quinoline (2.36 g, 7.23 mmol, 47.64% yield) was obtained as a pale yellow solid. ¹H NMR (400 MHz, CHLOROFORM-d) δ 8.73 (s, 1H), 8.21 (d, J=2.3 Hz, 1H), 8.00 (s, 1H), 7.62 (dd, J=2.1, 8.9 Hz, 1H), 4.14 (dd, J=4.1, 11.4 Hz, 2H), 3.61 (dt, J=1.7, 11.7 Hz, 2H), 3.52 (tt, J=3.7, 12.1 Hz, 1H), 2.02-1.93 (m, 2H), 1.86-1.81 (m, 2H). MS (M+H)⁺=32 8.00.

### Synthesis of methyl 5-chloro-2-[(6-chloro-3-tetrahydropyran-4-yl-4-quinolyl)amino]benzoate (6A)

A mixture of 4-bromo-6-chloro-3-tetrahydropyran-4-yl-quinoline (2.3 g, 7.04 mmol, 1 eq), methyl 2-amino-5-chloro-benzoate (1.31 g, 7.04 mmol, 1 eq), BrettPhos Pd G3 (638.35 mg, 704.19 umol, 0.1 eq), Cs₂CO₃ (4.59 g, 14.08 mmol, 2 eq) in dioxane (40 mL) was degassed and purged with N₂ for 3 times, and then the mixture was stirred at 105° C. for 12 h under N₂ atmosphere. LCMS showed the starting material was consumed completely and desired MS was detected. 10 mL of water was added to the mixture and extracted with Ethyl acetate (50 mL*3). The combined organic layers were washed with brine (10 mL), dried over Na₂SO₄ and concentrated to dryness to give residue. The crude product was purified by flash column (ISCO 20 g silica, 0-67% ethyl acetate in petroleum ether, gradient over 20 min). Compound methyl 5-chloro-2-[(6-chloro-3-tetrahydropyran-4-yl-4-quinolyl)amino]benzoate (880 mg, 2.04 mmol, 28.97% yield) was obtained as a yellow oil. MS (M+H)⁺=431.0

### Synthesis of 5-chloro-2-[(6-chloro-3-tetrahydropyran-4-yl-4-quinolyl)amino]benzoic acid (296A)

To a solution of methyl 5-chloro-2-[(6-chloro-3-tetrahydropyran-4-yl-4-quinolyl)amino]benzoate (880 mg, 2.04 mmol, 1 eq) in THF (7 mL), MeOH (2.1 mL) and H₂O (0.7 mL) was added LiOH·H₂O (171.24 mg, 4.08 mmol, 2 eq). The mixture was stirred at 60° C. for 2 h. LCMS showed the starting material was consumed completely and desired MS was detected. The reaction mixture was concentrated to dryness to give the crude product. 5 mL of water was added to the reaction, then the aqueous was acidified with 2M HCl at 0° C. until pH=5˜6. Then the mixture extracted with Ethyl acetate (15 mL*3). The combined organic layers were washed with brine (2 mL), dried over Na₂SO₄ and concentrated to dryness to give residue. The crude product was purified by flash column (ISCO 20 g silica, 0-100% ethyl acetate in petroleum ether; 0-13% methanol in dichloromethane, gradient over 20 min). Compound 5-chloro-2-[(6-chloro-3-tetrahydropyran-4-yl-4-quinolyl)amino]benzoic acid (359.80 mg, 831.82 μmol, 40.77% yield) was obtained as a yellow solid. ¹H NMR (400 MHz, DMSO-d₆) δ 9.76 (br s, 1H), 9.07 (s, 1H), 8.09 (d, J=8.9 Hz, 1H), 7.90 (d, J=2.6 Hz, 1H), 7.79-7.70 (m, 2H), 7.26 (dd, J=2.7, 8.9 Hz, 1H), 6.11 (d, J=9.0 Hz, 1H), 3.94 (dt, J=3.0, 11.8 Hz, 2H), 3.22-3.02 (m, 3H), 2.07-1.96 (m, 1H), 1.91-1.80 (m, 1H), 1.70 (br d, J=12.5 Hz, 1H), 1.54 (br d, J=12.9 Hz, 1H). MS (M+H)⁺=417.0

### Example 94—Synthesis of 297A

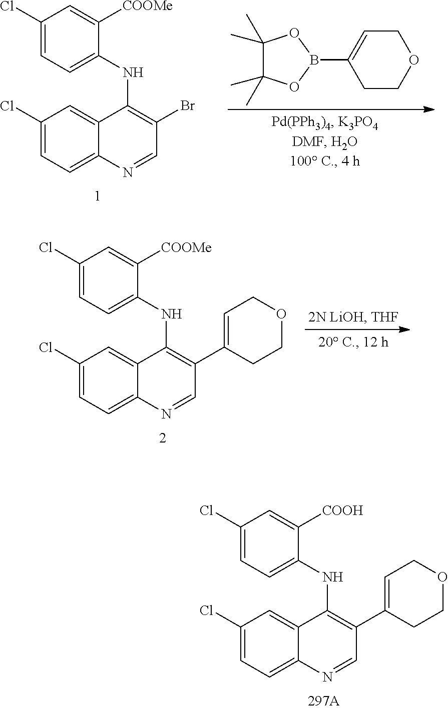 [SMILES: COC(=O)c1cc(Cl)ccc1Nc1c(Br)cnc2ccc(Cl)cc12 ; CC1(C)OB(C2=CCOCC2)OC1(C)C ; COC(=O)c1cc(Cl)ccc1Nc1c(C2=CCOCC2)cnc2ccc(Cl)cc12 ; O=C(O)c1cc(Cl)ccc1Nc1c(C2=CCOCC2)cnc2ccc(Cl)cc12]

Step 1. Synthesis of methyl 5-chloro-2-[[6-chloro-3-(3,6-dihydro-2H-pyran-4-yl)-4-quinolyl]amino]benzoate (2): To a stirred solution of methyl 2-[(3-bromo-6-chloro-4-quinolyl)amino]-5-chloro-benzoate (1.1 g, 2.58 mmol, 1 eq) in DMF (15 mL) and H₂O (3 mL) was added 2-(3,6-dihydro-2H-pyran-4-yl)-4,4,5,5-tetramethyl-1,3,2-dioxaborolane (596.57 mg, 2.84 mmol, 1.1 eq), K₃PO₄ (1.64 g, 7.74 mmol, 3 eq) and Pd(PPh₃)₄ (298.32 mg, 258.16 umol, 0.1 eq), the mixture was bubbled with N₂ for 1 minute, and stirred at 100° C. for 2 h. LCMS showed the starting material was consumed completely and desired MS was detected. The reaction mixture was poured into water (100 mL). The aqueous phase was extracted with ethyl acetate (100 mL*2). The combined organic phase was dried with anhydrous Na₂SO₄, filtered and concentrated in vacuum. The crude product was purified by flash column (ISCO 10 g silica, 0-10% ethyl acetate in petroleum ether, gradient over 20 min). Based on TLC (Petroleum ether:Ethyl acetate=0/1, R_{f}=0.61). methyl 5-chloro-2-[[6-chloro-3-(3,6-dihydro-2H-pyran-4-yl)-4-quinolyl]amino]benzoate (0.7 g, 1.63 mmol, 63.16% yield) was obtained as a yellow solid. MS (M+H)⁺=429.2.

Step 2. Synthesis of 5-chloro-2-[[6-chloro-3-(3,6-dihydro-2H-pyran-4-yl)-4-quinolyl]amino]benzoic acid (297A): A solution of methyl 5-chloro-2-[[6-chloro-3-(3,6-dihydro-2H-pyran-4-yl)-4-quinolyl]amino]benzoate (50 mg, 116.47 umol, 1 eq) and LiOH·H₂O (2 M, 116.47 uL, 2 eq) in THF (0.5 mL) and MeOH (0.5 mL) was stirred at 25° C. for 12 h. LCMS showed the starting material was consumed completely and desired Ms was detected. The reaction mixture was adjusted pH˜4 by adding 2N HCl. The mixture was purified by prep-HPLC (Phenomenex luna C18 80*40 mm*3 um; mobile phase: [water(0.04% HCl)-ACN]; B %: 18%-52%,7 min). Compound 5-chloro-2-[[6-chloro-3-(3,6-dihydro-2H-pyran-4-yl)-4-quinolyl]amino]benzoic acid (9.7 mg, 21.24 umol, 18.24% yield, 98.91% purity, HCl) was obtained as a yellow solid. ¹H NMR (400 MHz, DMSO-d6) δ=10.24 (br d, J=1.6 Hz, 1H), 8.75-8.69 (m, 1H), 8.66-8.53 (m, 1H), 8.19-8.10 (m, 1H), 8.00 (br d, J=8.4 Hz, 1H), 7.90 (d, J=2.6 Hz, 1H), 7.56 (br d, J=8.3 Hz, 1H), 7.02 (br s, 1H), 5.81 (br s, 1H), 3.89 (br s, 2H), 3.24 (br s, 2H), 2.09 (br d, J=3.1 Hz, 2H). MS (M+H)¹=414.9.

### Example 95—Synthesis of Compound 298A

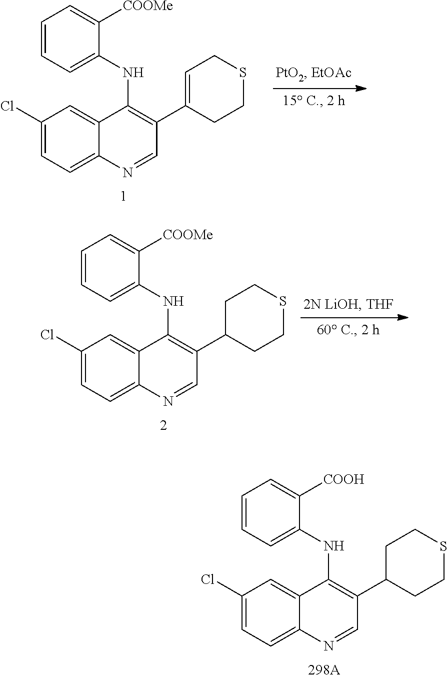 [SMILES: COC(=O)c1ccccc1Nc1c(C2=CCSCC2)cnc2ccc(Cl)cc12 ; COC(=O)c1ccccc1Nc1c(C2CCSCC2)cnc2ccc(Cl)cc12 ; O=C(O)c1ccccc1Nc1c(C2CCSCC2)cnc2ccc(Cl)cc12]

Step 1. Synthesis of methyl 2-[(6-chloro-3-tetrahydrothiopyran-4-yl-4-quinolyl)amino]benzoate (2): To a stirred solution of methyl 2-[[6-chloro-3-(3,6-dihydro-2H-thiopyran-4-yl)-4-quinolyl]amino]benzoate (300 mg, 730.08 umol, 1 eq) in EtOAc (5 mL) was added PtO2 (331.57 mg, 1.46 mmol, 2 eq) at 25° C., then the mixture was purged with H₂ for 3 times, and stirred at 25° C. for 2 h under H₂(15 psi). LCMS showed 45% starting material was remained and 20% desired product was detected. The reaction mixture was filtered, and filtrate was concentrate in vacuum. The residue was purified by prep-HPLC (column: Phenomenex luna C18 80*40 mm*3 um; mobile phase: [water(0.04% HCl)-ACN]; B %: 43%-63%,7 min). Compound methyl 2-[(6-chloro-3-tetrahydrothiopyran-4-yl-4-quinolyl)amino]benzoate (30 mg, 72.65 umol, 9.95% yield) was obtained as a yellow solid. MS (M+H)⁺=413.2.

Step 2. Synthesis of 2-[(6-chloro-3-tetrahydrothiopyran-4-yl-4-quinolyl)amino]benzoic acid (298A): To a stirred solution of methyl 2-[(6-chloro-3-tetrahydrothiopyran-4-yl-4-quinolyl)amino]benzoate (10 mg, 24.22 umol, 1 eq) in THF (0.3 mL) and MeOH (0.3 mL) was added LiOH·H₂O (2.03 mg, 48.43 umol, 2 eq) at 25° C., then the mixture was stirred at 60° C. for 2 h. LCMS showed the starting material was consumed completely and desired product was detected. The reaction mixture was adjusted pH-4 by 2N HCl. Then the mixture was concentrate in vacuum. The residue was purified prep-HPLC (column: Phenomenex Luna 80*30 mm*3 um; mobile phase: [water(HCl)-ACN]; B %: 20%-60%,8 min). Compound 2-[(6-chloro-3-tetrahydrothiopyran-4-yl-4-quinolyl)amino]benzoic acid (6.3 mg, 14.47 umol, 59.75% yield, 100% purity, HCl) was obtained as a yellow solid. ¹H NMR (400 MHz, DMSO-d6) δ=10.14-10.03 (m, 1H), 8.93 (s, 1H), 8.11 (d, J=9.0 Hz, 1H), 7.99 (dd, J=1.6, 7.8 Hz, 1H), 7.94-7.87 (m, 2H), 7.52-7.39 (m, 1H), 7.23-7.10 (m, 1H), 6.83-6.70 (m, 1H), 2.85-2.77 (m, 1H), 2.69-2.59 (m, 4H), 2.05-1.95 (m, 3H), 1.89-1.74 (m, 1H). MS (M+H)⁺=399.0

### Example 96—Synthesis of Compound 299A

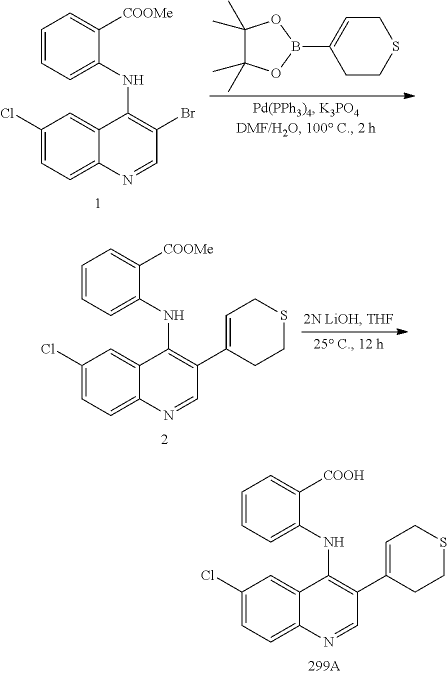 [SMILES: COC(=O)c1ccccc1Nc1c(Br)cnc2ccc(Cl)cc12 ; CC1(C)OB(C2=CCSCC2)OC1(C)C ; COC(=O)c1ccccc1Nc1c(C2=CCSCC2)cnc2ccc(Cl)cc12 ; O=C(O)c1ccccc1Nc1c(C2=CCSCC2)cnc2ccc(Cl)cc12]

Step 1. Synthesis of methyl 2-[[6-chloro-3-(3,6-dihydro-2H-thiopyran-4-yl)-4-quinolyl]amino]benzoate (2): To a stirred solution of methyl 2-[(3-bromo-6-chloro-4-quinolyl)amino]benzoate (1.2 g, 3.06 mmol, 1 eq) in DMF (10 mL) and H₂O (2 mL) was added 2-(3,6-dihydro-2H-thiopyran-4-yl)-4,4,5,5-tetramethyl-1,3,2-dioxaborolane (692.90 mg, 3.06 mmol, 1 eq), K₃PO₄ (1.95 g, 9.19 mmol, 3 eq) and Pd(PPh₃)₄ (354.06 mg, 306.40 umol, 0.1 eq), the mixture was purged with N₂ for 3 times, and stirred at 100° C. for 2 h. LCMS showed the starting material was consumed completely and the Ms of desired product was detected. The reaction mixture was poured into water (50 mL). The aqueous phase was extracted with ethyl acetate (50 mL×2). The combined organic phase was dried with anhydrous Na₂SO₄, filtered and concentrated in vacuum. The crude product was purified by flash column (ISCO 20 g silica, 0-10% ethyl acetate in petroleum ether, gradient over 20 min). Based on TLC (Petroleum ether:Ethyl acetate=3/1, R_{f}=0.60). Compound methyl 2-[[6-chloro-3-(3,6-dihydro-2H-thiopyran-4-yl)-4-quinolyl]amino]benzoate (0.7 g, 1.70 mmol, 55.60% yield) was obtained as a yellow solid. MS (M+H)⁺=411.2.

Step 2. Synthesis of 2-[[6-chloro-3-(3,6-dihydro-2H-thiopyran-4-yl)-4-quinolyl]amino]benzoic acid (299A): To a stirred solution of methyl 2-[[6-chloro-3-(3,6-dihydro-2H-thiopyran-4-yl)-4-quinolyl]amino]benzoate (50 mg, 121.68 umol, 1 eq) in THF (0.5 mL) an d MeOH (0.5 mL) was added LiOH·H₂O (2 M, 121.68 uL, 2 eq) at 25° C., then the mixture was stirred at 25° C. for 12 h. LCMS showed the starting material was consumed completely and desired Ms was detected. The reaction was adjusted pH-4 by adding 2N HCl. The mixture was purified by prep-HPLC (column: Phenomenex Luna 80*30 mm*3 um; mobile phase: [water(HCl)-ACN]; B %: 20%-55%,8 min). Compound 2-[[6-chloro-3-(3,6-dihydro-2H-thiopyran-4-yl)-4-quinolyl]amino]benzoic acid (18.30 mg, 42.23 umol, 34.71% yield, 100% purity, HCl) was obtained as a yellow solid. ¹H NMR (400 MHz, DMSO-d6) δ=10.25 (br s, 1H), 8.63 (s, 1H), 8.58 (br s, 1H), 8.10 (d, J=9.0 Hz, 1H), 8.02-7.93 (m, 2H), 7.51 (br t, J=7.8 Hz, 1H), 7.26 (br t, J=7.3 Hz, 1H), 6.99 (br d, J=4.3 Hz, 1H), 5.89 (br s, 1H), 2.96 (br s,2H), 2.29-2.04 (m, 4H). MS (M+H)⁺=397.0.

### Example 97—Synthesis of Compound 300A

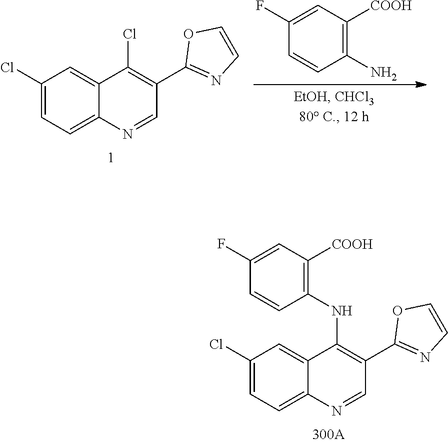

A solution of 2-amino-5-fluoro-benzoic acid (17.56 mg, 113.17 umol, 1 eq) and 2-(4,6-dichloro-3-quinolyl)oxazole (30 mg, 113.17 umol, 1 eq) in EtOH (0.5 mL) and CHCl₃ (0.1 mL) was stirred at 80° C. for 12 h. LCMS showed the starting material was consumed completely and the Ms of desired product was detected. The reaction mixture was concentrate in vacuum. The residue was purified by prep-HPLC (column: Phenomenex luna C18 80*40 mm*3 um; mobile phase: [water(0.04% HCl)-ACN]; B %: 16%-40%,7 min). Compound 2-[(6-chloro-3-oxazol-2-yl-4-quinolyl)amino]-5-fluoro-benzoic acid (11.10 mg, 26.03 umol, 23.01% yield, 98.56% purity, HCl) was obtained as a yellow solid. ¹H NMR (400 MHz, DMSO-d6) δ=11.70 (br s, 1H), 9.30 (s, 1H), 8.28 (s, 1H), 8.17 (d, J=9.0 Hz, 1H), 7.98 (dd, J=1.8, 8.9 Hz, 1H), 7.83 (d, J=1.8 Hz, 1H), 7.76 (dd, J=3.0, 9.0 Hz, 1H), 7.48 (s, 1H), 7.36 (dt, J=2.9, 8.3 Hz, 1H), 7.17 (dd, J=4.8, 8.8 Hz, 1H). MS (M+H)⁺=384.0

### Example 98—Synthesis of Compound 301A

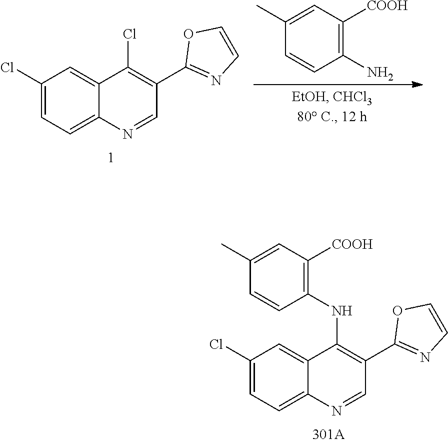

A solution of 2-(4,6-dichloro-3-quinolyl)oxazole (30 mg, 113.17 umol, 1 eq) and 2-amino-5-methyl-benzoic acid (17.11 mg, 113.17 umol, 1 eq) in EtOH (0.5 mL) and CHCl₃ (0.1 mL) was stirred at 80° C. for 12 h. LCMS showed the starting material was consumed completely and the Ms of desired product was detected. The reaction mixture was concentrate in vacuum. The residue was purified by prep-HPLC (column: Phenomenex luna C18 80*40 mm*3 um; mobile phase: [water(0.04% HCl)-ACN]; B %: 16%-40%,7 min) Compound 2-[(6-chloro-3-oxazol-2-yl-4-quinolyl)amino]-5-methyl-benzoic acid (14.10 mg, 33.32 umol, 29.44% yield, 98.36% purity, HCl) was obtained as a yellow solid. ¹H NMR (400 MHz, DMSO-d6) δ=11.85-11.67 (m, 1H), 9.34 (s, 1H), 8.32 (s, 1H), 8.12 (br d, J=9.0 Hz, 1H), 7.93 (dd, J=1.8, 9.1 Hz, 1H), 7.84 (s, 1H), 7.67 (d, J=2.1 Hz, 1H), 7.52 (s, 1H), 7.28 (br d, J=8.1 Hz, 1H), 7.01-6.92 (m, 1H), 2.35 (s, 3H). MS (M+H)⁺=380.0

### Example 99—Synthesis of Compound 302A

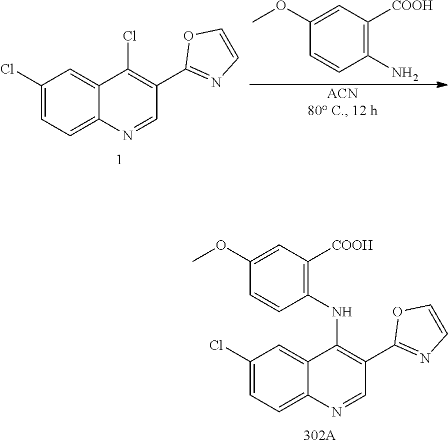

A solution of 2-(4,6-dichloro-3-quinolyl)oxazole (30 mg, 113.17 umol, 1 eq) and 2-amino-5-methoxy-benzoic acid (18.92 mg, 113.17 umol, 1 eq) in ACN (0.5 mL) was stirred at 80° C. for 12 h. LCMS showed the starting material was consumed completely and desired Ms was detected. The reaction mixture was concentrate in vacuum. The residue was purified by prep-HPLC (column: Phenomenex Luna C18 150*30 mm*5 um; mobile phase: [water(0.01% FA)-ACN]; B %: 10%-45%,8 min). Compound 2-[(6-chloro-3-oxazol-2-yl-4-quinolyl)amino]-5-methoxy-benzoic acid (10.80 mg, 23.74 umol, 20.98% yield 97.11% purity, FA) was obtained as a yellow solid. ¹H NMR (400 MHz, DMSO-d6+D₂O) δ=9.21-9.14 (m, 1H), 8.10 (s, 1H), 7.99-7.90 (m, 1H), 7.76-7.65 (m, 1H), 7.47 (d, J=2.3 Hz, 1H), 7.41 (d, J=3.0 Hz, 1H), 7.40 (s, 1H), 6.92 (dd, J=2.9, 8.9 Hz, 1H), 6.70 (d, J=8.9 Hz, 1H), 3.78-3.65 (m, 3H). MS (M+H)⁺=395.9.

### Example 100—Synthesis of Compound 303A

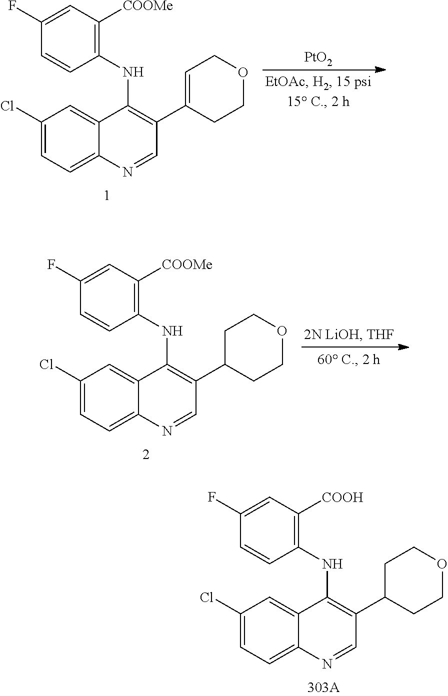

Step 1. Synthesis of methyl methyl 2-[(6-chloro-3-tetrahydropyran-4-yl-4-quinolyl)amino]-5-fluoro-benzoate (2): To a stirred solution of methyl 2-[[6-chloro-3-(3,6-dihydro-2H-pyran-4-yl)-4-quinolyl]amino]-5-fluoro-benzoate (150 mg, 363.34 umol, 1 eq) in EtOAc (3 mL) and AcOH (0.1 mL) was added PtO2 (82.51 mg, 363.34 umol, 1 eq) at 15° C., the n the mixture was purged with H₂ for 3 times, and stirred at 15° C. for 1 h under H₂(15 psi). LCMS showed the starting material was consumed completely and desired product was detected. The reaction mixture was filtered, and filtrate was concentrate in vacuum. The residue was purified by prep-HPLC (column: Phenomenex luna C18 80*40 mm*3 um; mobile phase: [water(0.04% HCl)-ACN]; B %: 30%-50%,7 min). Compound methyl 2-[(6-chloro-3-tetrahydropyran-4-yl-4-quinolyl)amino]-5-fluoro-benzoate (30 mg, 72.31 umol, 19.90% yield) was obtained as a yellow solid. MS (M+H)⁺=415.2.

Step 2. Synthesis of 2-[(6-chloro-3-tetrahydropyran-4-yl-4-quinolyl)amino]-5-fluoro-benzoic acid (303A): To a stirred solution of methyl 2-[(6-chloro-3-tetrahydropyran-4-yl-4-quinolyl)amino]-5-fluoro-benzoate (20 mg, 48.21 umol, 1 eq) in THF (2 mL) was added LiOH·H₂O (2 M, 48.21 uL, 2 eq) at 25° C., then the mixture was stirred at 60° C. for 2 h. LCMS showed the starting material was consumed completely and desired product was detected. The reaction mixture was adjusted pH˜4 by adding 2N HCl. Then the mixture was concentrate in vacuum. The residue was purified by prep-HPLC (column: Phenomenex Luna 80*30 mm*3 um; mobile phase: [water(0.04% HCl)-ACN]; B %: 10%-40%,8 min). Compound 2-[(6-chloro-3-tetrahydropyran-4-yl-4-quinolyl)amino]-5-fluoro-benzoic acid (10.30 mg. 23.55 umol, 48.86% yield, 100% purity, HCl) was obtained as a yellow solid. ¹H NMR (400 MHz, DMSO-d6+D₂O) δ=8.73 (s, 1H), 8.00 (d, J=9.1 Hz, 1H), 7.87 (dd, J=2.2, 9.1 Hz, 1H), 7.79 (d, J=2.1 Hz, 1H), 7.72 (dd, J=3.1, 9.0 Hz, 1H), 7.37 (ddd, J=3.1, 7.9, 8.9 Hz, 1H), 6.96 (dd, J=4.8, 9.0 Hz, 1H), 3.91-3.85 (m, 2H), 3.30-3.12 (m, 2H), 3.03-2.90 (m, 1H), 1.85 (td, J=2.0, 11.1 Hz, 1H), 1.74-1.57 (m, 3H). MS (M+H)⁺=401.0

### Example 101—Synthesis of 304A

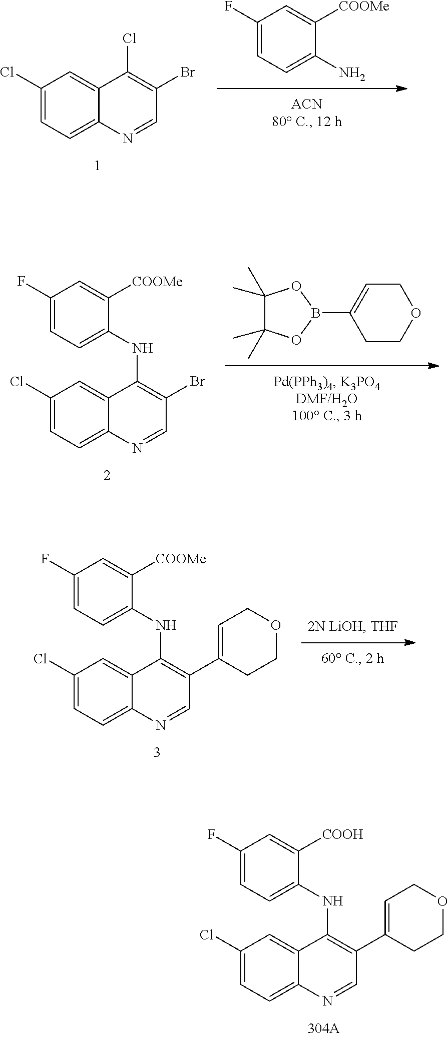 [SMILES: Clc1ccc2ncc(Br)c(Cl)c2c1 ; COC(=O)c1cc(F)ccc1N ; COC(=O)c1cc(F)ccc1Nc1c(Br)cnc2ccc(Cl)cc12 ; CC1(C)OB(C2=CCOCC2)OC1(C)C ; COC(=O)c1cc(F)ccc1Nc1c(C2=CCOCC2)cnc2ccc(Cl)cc12 ; O=C(O)c1cc(F)ccc1Nc1c(C2=CCOCC2)cnc2ccc(Cl)cc12]

Step 1. Synthesis of methyl 2-[(3-bromo-6-chloro-4-quinolyl)amino]-5-fluoro-benzoate (2): A solution of 3-bromo-4,6-dichloro-quinoline (1 g, 3.61 mmol, 1 eq) and methyl 2-amino-5-fluoro-benzoate (610.78 mg, 3.61 mmol, 1 eq) in ACN (20 mL) was stirred at 80° C. for 12 h. LCMS showed the starting material was consumed completely and desired product was detected. The reaction mixture was filtered, and filter cake was concentrate in vacuum. Compound methyl 2-[(3-bromo-6-chloro-4-quinolyl)amino]-5-fluoro-benzoate (1 g, 2.44 mmol, 67.61% yield) was obtained as a yellow solid. MS (M+H)⁺=411.1.

Step 2. Synthesis of methyl 2-[[6-chloro-3-(3,6-dihydro-2H-pyran-4-yl)-4-quinolyl]amino]-5-fluoro-benzoate (3): To a stirred solution of methyl 2-[(3-bromo-6-chloro-4-quinolyl)amino]-5-fluoro-benzoate (1 g, 2.44 mmol, 1 eq) in DMF (15 mL) and H₂O (3 mL) was added 2-(3,6-dihydro-2H-pyran-4-yl)-4,4,5,5-tetramethyl-1,3,2-dioxaborolane (512.84 mg, 2.44 mmol, 1 eq), K₃PO₄ (1.55 g. 7.32 mmol, 3 eq) and Pd(PPh₃)₄ (282.09 mg, 244.12 umol, 0.1 eq) at 25° C., then the mixture was purged with N₂ for 3 times, and stirred at 100° C. for 3 h. TLC (Petroleum ether/Ethyl acetate=1:1, R_{f}=0.40) showed starting material was consumed completely and new spot was formed. The reaction mixture was poured into water (50 mL). The aqueous phase was extracted with ethyl acetate (100 mL*2). The combined organic phase was dried with anhydrous Na₂SO₄, filtered and concentrated in vacuum. The residue was purified by flash column (ISCO 20 g silica, 60-70% Ethyl acetate in Petroleum ether, gradient over 15 min). Compound methyl 2-[[6-chloro-3-(3,6-dihydro-2H-pyran-4-yl)-4-quinolyl]amino]-5-fluoro-benzoate (500 mg, 1.21 mmol, 49.61% yield) was obtained as a yellow solid. MS (M+H)⁺=413.2.

Step 3. Synthesis of 2-[[6-chloro-3-(3,6-dihydro-2H-pyran-4-yl)-4-quinolyl]amino]-5-fluoro-benzoic acid (304A): To a stirred solution of methyl 2-[[6-chloro-3-(3,6-dihydro-2H-pyran-4-yl)-4-quinolyl]amino]-5-fluoro-benzoate (50 mg, 121.11 umol, 1 eq) in THF (0.5 mL) and MeOH (0.5 mL) was added LiOH·H₂O (2 M, 121.11 uL, 2 eq) at 25° C., then the mixture was stirred at 25° C. for 12 h. LCMS showed the starting material was consumed completely and desired product was detected. The reaction mixture was adjusted pH˜4 by adding 2N HCl. Then the mixture was concentrate in vacuum. The residue was purified by prep-HPLC (column: Phenomenex luna C18 80*40 mm*3 um; mobile phase: [water(0.04% HCl)-ACN]; B %: 20%-40%,7 min). Compound 2-[[6-chloro-3-(3,6-dihydro-2H-pyran-4-yl)-4-quinolyl]amino]-5-fluoro-benzoic acid (14.90 mg, 34.23 umol, 28.26% yield, 100% purity, HCl) was obtained as a yellow solid. 1H NMR (400 MHz, DMSO-d6) δ=10.35-10.18 (m, 1H), 8.73 (br d, J=13.5 Hz, 1H), 8.66-8.59 (m, 1H), 8.21-8.09 (m, 1H), 8.03 (br dd, J=1.9, 8.9 Hz, 1H), 7.70 (dd, J=3.0, 9.1 Hz, 1H), 7.54-7.37 (m, 1H), 7.21 (br d, J=4.8 Hz, 1H), 5.75 (br s, 1H), 3.85 (br s, 2H), 3.19 (br s, 2H), 2.05 (br s, 2H). MS (M+H)⁺=399.0.

### Example 102—Synthesis of Compound 305A

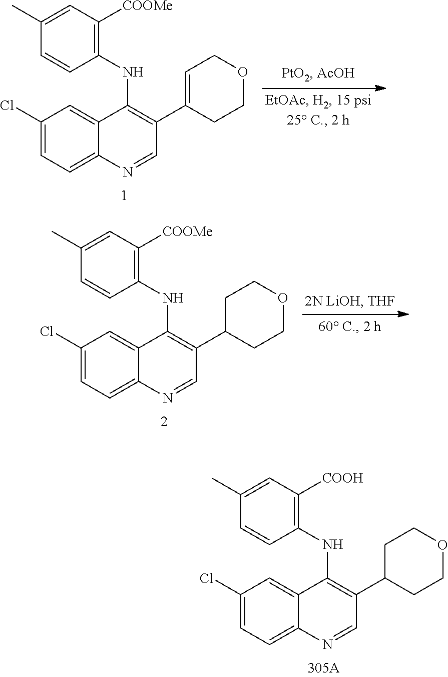 [SMILES: COC(=O)c1cc(C)ccc1Nc1c(C2=CCOCC2)cnc2ccc(Cl)cc12 ; COC(=O)c1cc(C)ccc1Nc1c(C2CCOCC2)cnc2ccc(Cl)cc12 ; Cc1ccc(Nc2c(C3CCOCC3)cnc3ccc(Cl)cc23)c(C(=O)O)c1]

Step 1. Synthesis of methyl 2-[(6-chloro-3-tetrahydropyran-4-yl-4-quinolyl)amino]-5-methyl-benzoate (2): To a stirred solution of methyl 2-[[6-chloro-3-(3,6-dihydro-2H-pyran-4-yl)-4-quinolyl]amino]-5-methyl-benzoate (150 mg, 366.86 umol, 1 eq) in AcOH (0.1 mL) and EtOAc (1 mL) was added PtO₂ (83.31 mg, 366.86 umol, 1 eq) at 25° C., then the mixture was purged with H₂ for 3 times, and stirred at 25° C. for 2 h under H₂(15 psi). LCMS showed the 10% starting material was remained and 40% desired product was detected. The reaction mixture was filtered, and filtrate was concentrate in vacuum. The residue was purified by prep-HPLC (column: Phenomenex luna C18 80*40 mm*3 um; mobile phase: [water(0.04% HCl)-ACN]; B %: 30%-45%,7 min). Compound methyl 2-[(6-chloro-3-tetrahydropyran-4-yl-4-quinolyl)amino]-5-methyl-benzoate (30 mg, 67.06 umol, 18.28% yield, HCl) was obtained as a yellow solid. MS (M+H)⁺=411.3.

Step 2. Synthesis of 2-[(6-chloro-3-tetrahydropyran-4-yl-4-quinolyl)amino]-5-methyl-benzoic acid (305A): To a stirred solution of methyl 2-[(6-chloro-3-tetrahydropyran-4-yl-4-quinolyl)amino]-5-methyl-benzoate (30 mg, 73.01 umol, 1 eq) in THF (3 mL) was added LiOH·H₂O (2 M, 73.01 uL, 2 eq) at 25° C., then the mixture was stirred at 60° C. for 2 h. LCMS showed the starting material was consumed completely and desired Ms was detected. HPLC The reaction mixture was adjusted pH-4 by adding 2N HCl. Then the mixture was concentrate in vacuum. The residue was purified by prep-HPLC (column: Phenomenex Luna 80*30 mm*3 um; mobile phase: [water(0.04% HCl)-ACN]; B %: 20%-45%,8 min). Compound 2-[(6-chloro-3-tetrahydropyran-4-yl-4-quinolyl)amino]-5-methyl-benzoic acid (10.50 mg, 24.23 umol, 33.19% yield, 100% purity, HCl) was obtained as a yellow solid. ¹H NMR (400 MHz, DMSO-d6) δ=10.06 (br s, 1H), 8.84 (s, 1H), 8.14 (d, J=9.5 Hz, 1H), 7.96-7.87 (m, 2H), 7.81 (d, J=1.6 Hz, 1H), 7.33 (br d, J=8.0 Hz, 1H), 6.86 (br d, J=5.8 Hz, 1H), 3.92 (br d, J=10.5 Hz, 2H), 3.27-3.16 (m, 2H), 3.07-2.97 (m, 1H), 2.35 (s, 3H), 1.92 (br d, J=10.1 Hz, 1H), 1.80-1.58 (m, 3H). MS (M+H)⁺=397.0

### Example 103—Synthesis of 306A

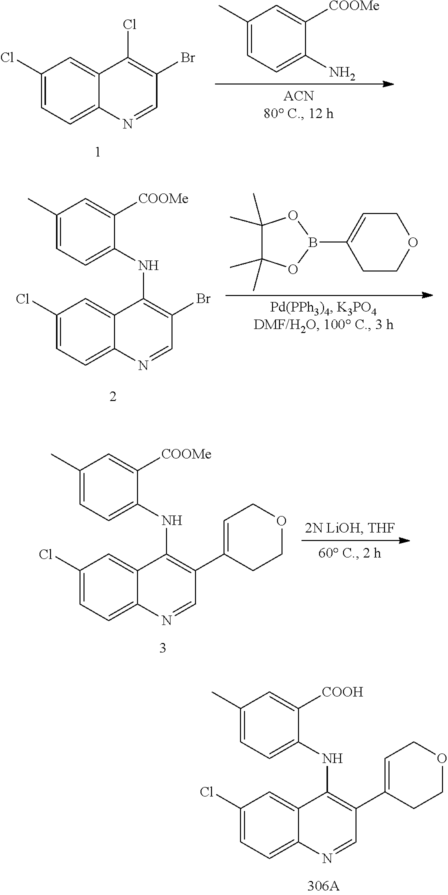

Step 1. Synthesis of methyl 2-[(3-bromo-6-chloro-4-quinolyl)amino]-5-methyl-benzoate (2): A solution of 3-bromo-4,6-dichloro-quinoline (1 g, 3.61 mmol, 1 eq) and methyl 2-amino-5-methyl-benzoate (596.47 mg, 3.61 mmol, 1 eq) in ACN (15 mL) was stirred at 80° C. for 12 h. LCMS showed the starting material was consumed completely and the Ms of desired product was detected. The reaction mixture was filtered, and filter cake was concentrate in vacuum. Compound methyl 2-[(3-bromo-6-chloro-4-quinolyl)amino]-5-methyl-benzoate (1.1 g, 2.71 mmol, 75.09% yield) was obtained as a yellow solid. MS (M+H)⁺=407.1.

Step 2. Synthesis of methyl 2-[[6-chloro-3-(3,6-dihydro-2H-pyran-4-yl)-4-quinolyl]amino]-5-methyl-benzoate (3): To a stirred solution of methyl 2-[(3-bromo-6-chloro-4-quinolyl)amino]-5-methyl-benzoate (1.1 g, 2.71 mmol, 1 eq) in DMF (15 mL) and H₂O (3 mL) was added 2-(3,6-dihydro-2H-pyran-4-yl)-4,4,5,5-tetramethyl-1,3,2-dioxaborolane (569.63 mg, 2.71 mmol, 1 eq), Pd(PPh₃)₄ (313.34 mg, 271.15 umol, 0.1 eq) and K₃PO₄ (1.73 g, 8.13 mmol, 3 eq), then the mixture was purged withN2 for 3 times, and stirred at 100° C. for 2 h. LCMS showed the starting material was consumed completely and desired Ms was detected. The reaction mixture was poured into water (50 mL) The aqueous phase was extracted with ethyl acetate (100 mL*2). The combined organic phase was dried with anhydrous Na₂SO₄, filtered and concentrated in vacuum. The crude product was purified by flash column (ISCO 10 g silica, 0-10% ethyl acetate in petroleum ether, gradient over 20 min). Based on TLC (Petroleum ether:Ethyl acetate=1/1, R_{f}=0.52). Compound methyl 2-[[6-chloro-3-(3,6-dihydro-2H-pyran-4-yl)-4-quinolyl]amino]-5-methyl-benzoate (0.5 g, 1.22 mmol, 45.10% yield) was obtained as a yellow oil. MS (M+H)⁺=409.3.

Step 3. Synthesis of 2-[[6-chloro-3-(3,6-dihydro-2H-pyran-4-yl)-4-quinolyl]amino]-5-methyl-benzoic acid (306A): To a stirred solution of methyl 2-[[6-chloro-3-(3,6-dihydro-2H-pyran-4-yl)-4-quinolyl]amino]-5-methyl-benzoate (50 mg, 122.29 umol, 1 eq) in THF (0.5 mL) and MeOH (0.5 mL) was added LiOH·H₂O (2 M, 122.29 uL, 2 eq) at 25° C., then the mixture was stirred at 25° C. for 12 h. LCMS showed the starting material was consumed completely and desired product was detected. The reaction mixture was adjusted pH-4 by adding 2N HCl. Then the mixture was concentrate in vacuum. The residue was purified by prep-HPLC (column: Phenomenex luna C18 80*40 mm*3 um; mobile phase: [water(0.04% HCl)-ACN]; B %: 25%-40%,6 min). Compound 2-[[6-chloro-3-(3,6-dihydro-2H-pyran-4-yl)-4-quinolyl]amino]-5-methyl-benzoic acid (23.60 mg, 54.72 umol, 44.74% yield, 100% purity, HCl) was obtained as a yellow solid. ¹H NMR (400 MHz, DMSO-d6) δ=10.29 (br s, 1H), 8.69 (br s, 1H), 8.58 (s, 1H), 8.18 (d, J=9.0 Hz, 1H), 8.00 (br d, J=9.0 Hz, 1H), 7.77 (s, 1H), 7.34 (br d, J=8.1 Hz, 1H), 6.99 (br d, J=8.1 Hz, 1H), 5.74 (br s, 1H), 3.83 (br s, 2H), 3.14 (br s, 2H), 2.36 (s, 3H), 2.04 (br s, 2H). MS (M+H)⁺=395.0.

### Example 104—Synthesis of Compound 307A

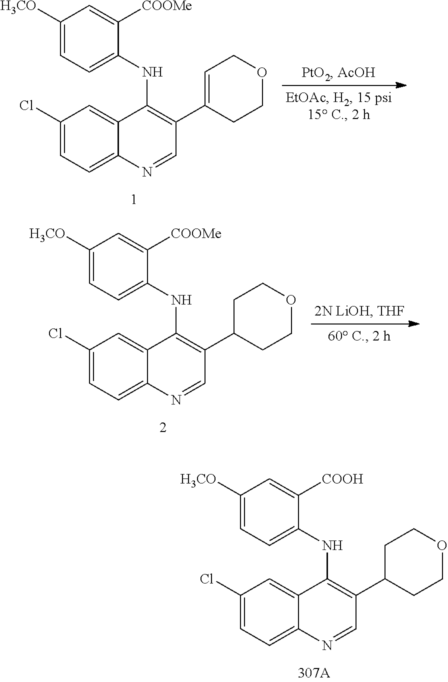 [SMILES: COC(=O)c1cc(OC)ccc1Nc1c(C2=CCOCC2)cnc2ccc(Cl)cc12 ; COC(=O)c1cc(OC)ccc1Nc1c(C2CCOCC2)cnc2ccc(Cl)cc12 ; COc1ccc(Nc2c(C3CCOCC3)cnc3ccc(Cl)cc23)c(C(=O)O)c1]

Step 1. Synthesis of methyl 2-[(6-chloro-3-tetrahydropyran-4-yl-4-quinolyl)amino]-5-methoxy-benzoate (2): To a stirred solution of methyl 2-[[6-chloro-3-(3,6-dihydro-2H-pyran-4-yl)-4-quinolyl]amino]-5-methoxy-benzoate (200 mg, 470.73 umol, 1 eq) in EtOAc (2 mL) and AcOH (0.2 mL) was added PtO2 (106.89 mg, 470.73 umol, 1 eq) at 25° C., then the mixture was purged with H₂ for 3 times, and stirred at 25° C. for 2 h under H₂(15 psi). LCMS showed the starting material was consumed completely and desired Ms was detected. The reaction mixture was filtered, and filtrate was concentrate in vacuum. The residue was purified by prep-HPLC (column: Phenomenex Luna C18 200*40 mm*10 um; mobile phase: [water(0.1% FA)-ACN]; B %: 20%-50%,8 min). Compound methyl 2-[(6-chloro-3-tetrahydropyran-4-yl-4-quinolyl)amino]-5-methoxy-benzoate (25 mg, 58.56 umol, 12.44% yield) was obtained as a yellow solid. MS (M+H)⁺=427.2.

Step 2. Synthesis of 2-[(6-chloro-3-tetrahydropyran-4-yl-4-quinolyl)amino]-5-methoxy-benzoic acid (307A): To a stirred solution of methyl 2-[(6-chloro-3-tetrahydropyran-4-yl-4-quinolyl)amino]-5-methoxy-benzoate (25 mg, 58.56 umol, 1 eq) in THF (0.3 mL) and MeOH (0.3 mL) was added LiOH·H₂O (2 M, 58.56 uL, 2 eq) at 25° C., then the mixture was stirred at 25° C. for 12 h. LCMS showed the starting material was consumed completely a nd desired product was detected. The reaction mixture was adjusted pH˜4 by adding 2N HC 1. The mixture was purified by prep-HPLC (column: Phenomenex Luna C18 100*30 mm*5 um; mobile phase: [water(FA)-ACN]; B %: 20%-60%,10 min). Compound 2-[(6-chloro-3-tetrahydropyran-4-yl-4-quinolyl)amino]-5-methoxy-benzoic acid (4.5 mg, 9.81 umol, 16.74% yield, 100% purity, FA) was obtained as a yellow solid. ¹H NMR (400 MHz, DMSO-d6) δ=9 0.57-9.45 (m, 1H), 9.01 (s, 1H), 8.09-8.04 (m, 1H), 7.75-7.72 (m, 1H), 7.72 (s, 1H), 7.46 (d, J=3.0 Hz, 1H), 6.91 (dd, J=3.1, 9.1 Hz, 1H), 6.12 (d, J=9.0 Hz, 1H), 3.94 (br d, J=10.1 Hz, 2H), 3.71 (s, 3H), 3.15 (dt, J=3.4, 11.9 Hz, 3H), 2.10-1.95 (m, 1H), 1.92-1.78 (m, 1H), 1.75-1.50 (m, 2H). MS (M+H)⁺=413.1

### Example 105—Synthesis of 308A

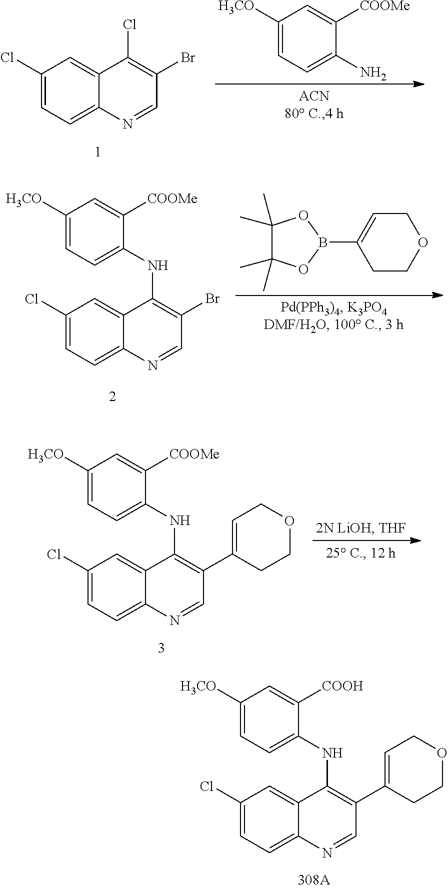

Step 1. Synthesis of methyl 2-[(3-bromo-6-chloro-4-quinolyl)amino]-5-methoxy-benzoate (2): A solution of 3-bromo-4,6-dichloro-quinoline (1 g, 3.61 mmol, 1 eq) and methyl 2-amino-5-methoxy-benzoate (654.24 mg, 3.61 mmol, 1 eq) in ACN (15 mL) was stirred at 80° C. for 4 h. LCMS showed the starting material was consumed completely and desired Ms was detected. The reaction mixture was filtered, and filter cake was concentrate in vacuum. Compound methyl 2-[(3-bromo-6-chloro-4-quinolyl)amino]-5-methoxy-benzoate (1 g, 2.37 mmol, 65.68% yield) was obtained as a yellow solid. MS (M+H)⁺=423.1.

Step 2. Synthesis of methyl 2-[[6-chloro-3-(3,6-dihydro-2H-pyran-4-yl)-4-quinolyl]amino]-5-methoxy-benzoate (3): To a stirred solution of methyl 2-[(3-bromo-6-chloro-4-quinolyl)amino]-5-methoxy-benzoate (1.1 g, 2.61 mmol, 1 eq) in DMF (15 mL) and H₂O (3 mL) was added 2-(3,6-dihydro-2H-pyran-4-yl)-4,4,5,5-tetramethyl-1,3,2-dioxaborolane (548.02 mg, 2.61 mmol, 1 eq), K₃PO₄ (1.66 g, 7.83 mmol, 3 eq) and and Pd(PPh₃)₄ (301.45 mg, 260.87 umol, 0.1 eq), the mixture was purged with N₂ for 3 times, and stirred at 100° C. for 2 h. TLC (Petroleum ether/Ethyl acetate=3:1, R_{f}=0.36) showed starting material was consumed completely and new spot was formed. The reaction mixture was poured into water (50 mL). The aqueous phase was extracted with ethyl acetate (100 mL*2). The combined organic phase was dried with anhydrous Na₂SO₄, filtered and concentrated in vacuum. The residue was purified by flash column (ISCO 20 g silica, 15-20% Ethyl acetate in Petroleum ether, gradient over 15 min). Compound methyl 2-[[6-chloro-3-(3,6-dihydro-2H-pyran-4-yl)-4-quinolyl]amino]-5-methoxy-benzoate (300 mg, 706.09 umol, 27.07% yield) was obtained as a yellow solid. MS (M+H)⁺=425.2.

Step 3. Synthesis of 2-[[6-chloro-3-(3,6-dihydro-2H-pyran-4-yl)-4-quinolyl]amino]-5-methoxy-benzoic acid (308A): To a stirred solution of methyl 2-[[6-chloro-3-(3,6-dihydro-2H-pyran-4-yl)-4-quinolyl]amino]-5-methoxy-benzoate (50 mg, 117.68 umol, 1 eq) in THF (0.3 mL) and MeOH (0.3 mL) was added LiOH·H₂O (2 M, 117.68 uL, 2 eq) at 25° C., then the mixture was stirred at 25° C. for 12 h. LCMS showed the starting material was consumed completely and desired Ms was detected. The reaction mixture was adjusted pH-4 by adding 2N HCl. Then the mixture was concentrate in vacuum. The residue was purified by prep-HPLC (column: Phenomenex luna C18 80*40 mm*3 um; mobile phase: [water(0.04% HCl)-ACN]; B %: 16%-36%,7 min). Compound 2-[[6-chloro-3-(3,6-dihydro-2H-pyran-4-yl)-4-quinolyl]amino]-5-methoxy-benzoic acid (14.80 mg, 33.09 umol, 28.12% yield, 100% purity, HCl) was obtained as a yellow solid. ¹H NMR (400 MHz, DMSO-d6) δ=10.28 (br s, 1H), 8.79 (br s, 1H), 8.53 (br d, J=4.0 Hz, 1H), 8.17-7.96 (m, 2H), 7.44 (d, J=1.5 Hz, 1H), 7.18 (br s, 2H), 5.70 (br s, 1H), 3.85 (s, 5H), 3.14 (br s, 2H), 2.03 (br s, 2H). MS (M+H)⁺=411.0.

### Example 106—Synthesis of Compound 318A

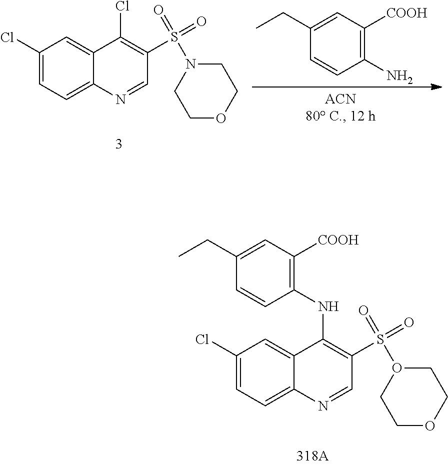

A solution of 2-amino-5-ethyl-benzoic acid (23.79 mg, 144.00 umol, 1 eq) and 4-[(4,6-dichloro-3-quinolyl)sulfonyl]morpholine (50 mg, 144.00 umol, 1 eq) in ACN (1 mL) was stirred at 80° C. for 12 h. LCMS showed the starting material was consumed completely and desired Ms was detected. The reaction mixture was concentrate in vacuum. The residue was purified by prep-HPLC (column: Phenomenex luna C18 80*40 mm*3 um; mobile phase: [water (0.04% HCl)-ACN]; B %: 34%-62%,7 min). Compound 2-[(6-chloro-3-morpholinosulfony 1-4-quinolyl)amino]-5-ethyl-benzoic acid (36.30 mg, 70.84 umol, 49.20% yield, 100% purity, HCl) was obtained as a yellow solid. ¹H NMR (400 MHz, DMSO-d6+D₂O) δ=8.99 (s, 1H), 8.04 (d, J=9.0 Hz, 1H), 7.85 (dd, J=2.4, 9.0 Hz, 1H), 7.81 (d, J=2.1 Hz, 1H), 7.47 (d, J=2.3 Hz, 1H), 7.21 (dd, J=2.2, 8.4 Hz, 1H), 6.63 (d, J=8.5 Hz, 1H), 3.52-3.42 (m, 2H), 3.39-3.30 (m, 2H), 3.10-2.93 (m, 4H), 2.59-2.53 (m, 2H), 1.12 (t, J=7.6 Hz, 3H). MS (M+H)⁺=476.0

### Example 107—Synthesis of Compound 319A

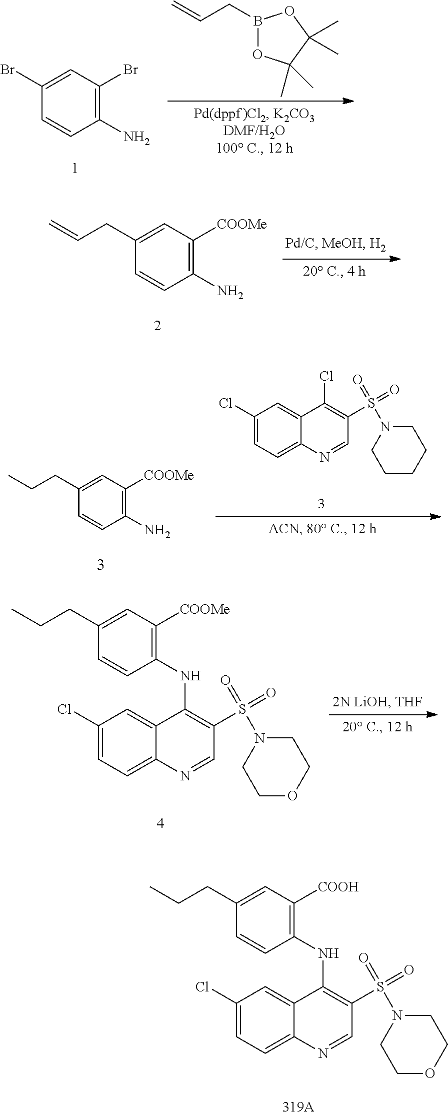

Step 1. Synthesis of methyl 5-allyl-2-amino-benzoate (2): To a stirred solution of methyl 2-amino-5-bromo-benzoate (1 g, 4.35 mmol, 1 eq) in DMF (10 mL) and H₂O (2 mL) was added 2-allyl-4,4,5,5-tetramethyl-1,3,2-dioxaborolane (730.43 mg, 4.35 mmol, 1 eq) Pd(dppf)Cl₂ (318.05 mg, 434.67 umol, 0.1 eq) K₂CO₃ (1.80 g, 13.04 mmol, 3 eq) at 25° C., then the mixture was bubbled with N₂ for 3 times, and stirred at 100° C. for 3 h. LCMS showed the starting material was consumed completely and desired product was detected. The reaction mixture was poured into water (50 mL). The aqueous phase was extracted with ethyl acetate (50 mL*3). The combined organic phase was dried with anhydrous Na₂SO₄, filtered an d concentrated in vacuum. The crude product was purified by flash column (ISCO 10 g silica, 0-10% ethyl acetate in petroleum ether, gradient over 20 min). Based on TLC (Petroleum ether:Ethyl acetate=3/1, R_{f}=0.51). Compound methyl 5-allyl-2-amino-benzoate (400 mg, 1.26 mmol, 28.87% yield, 60% purity) was obtained as a yellow oil. MS (M+H)⁺=192.0.

Step 2. Synthesis of methyl 2-amino-5-propyl-benzoate (3): To a stirred solution of methyl 5-allyl-2-amino-benzoate (100 mg, 522.94 umol, 1 eq) in MeOH (2 mL) was added Pd/C (522.94 ug, 522.94 umol, 10% purity, 1 eq) at 25° C., then the mixture was purged with H₂ for 3 times, and stirred at 25° C. for 12 h under H₂(15 psi). LCMS showed the starting material was consumed completely and desired Ms was detected. The reaction mixture was filtered, and filtrate was concentrate in vacuum Compound methyl 2-amino-5-propyl-benzoate (100 mg, 517.49 umol, 98.96% yield) was obtained as a yellow oil. MS (M+H)⁺=194.2

Step 3. Synthesis of methyl 2-[(6-chloro-3-morpholinosulfonyl-4-quinolyl)amino]-5-propyl-benzoate (4): A solution of methyl 2-amino-5-propyl-benzoate (30 mg, 155.25 umol, 1 eq) and 4-[(4,6-dichloro-3-quinolyl)sulfonyl]morpholine (53.90 mg, 155.25 umol, 1 eq) in ACN (1 mL) was stirred at 80° C. for 12 h. LCMS showed the starting material was consumed completely and desired product was detected. The reaction mixture was concentrate in vacuum. Compound methyl 2-[(6-chloro-3-morpholinosulfonyl-4-quinolyl)amino]-5-propyl-benzoate (80 mg, crude) was obtained as a yellow solid. MS (M+H)⁺=504.0.

Step 4. Synthesis of 2-[(6-chloro-3-morpholinosulfonyl-4-quinolyl)amino]-5-prop yl-benzoic acid (319A): To a stirred solution of methyl 2-[(6-chloro-3-morpholinosulfonyl-4-quinolyl)amino]-5-propyl-benzoate (80 mg, 158.73 umol, 1 eq) in THF (0.3 mL) and MeOH (0.3 mL) was added LiOH·H₂O (2 M, 158.73 uL, 2 eq) at 25° C., then the mixture was stirred at 25° C. for 12 h. LCMS showed the starting material was consumed completely and the Ms of desired product was detected. The reaction mixture was adjusted pH˜4 by adding 2N HCl. The mixture was purified by prep-HPLC (column: Phenomenex Luna 80*30 mm*3 um; mobile phase: [water(0.04% HCl)-ACN]; B %: 35%-70%,8 min). Compound 2-[(6-chloro-3-morpholinosulfonyl-4-quinolyl)amino]-5-propyl-benzoic acid (16.1 mg, 29.74 umol, 18.74% yield, 97.25% purity, HCl) was obtained as a yellow solid.

¹H NMR (400 MHz, DMSO-d6) δ=10.29 (br s, 1H), 9.05 (s, 1H), 8.10 (d, J=9.0 Hz, 1H), 7.88 (dd, J=2.3, 9.0 Hz, 1H), 7.81 (d, J=2.0 Hz, 1H), 7.54 (d, J=2.1 Hz, 1H), 7.20 (dd, J=2.1, 8.4 Hz, 1H), 6.68 (d, J=8.4 Hz, 1H), 3.51 (br d, J=8.6 Hz, 4H), 3.09-3.00 (m, 4H), 2.59-2.54 (m, 2H), 1.63-1.52 (m, 2H), 0.86 (t, J=7.3 Hz, 3H). MS (M+H)⁺=490.0

### Example 108—Synthesis of Compound 320A

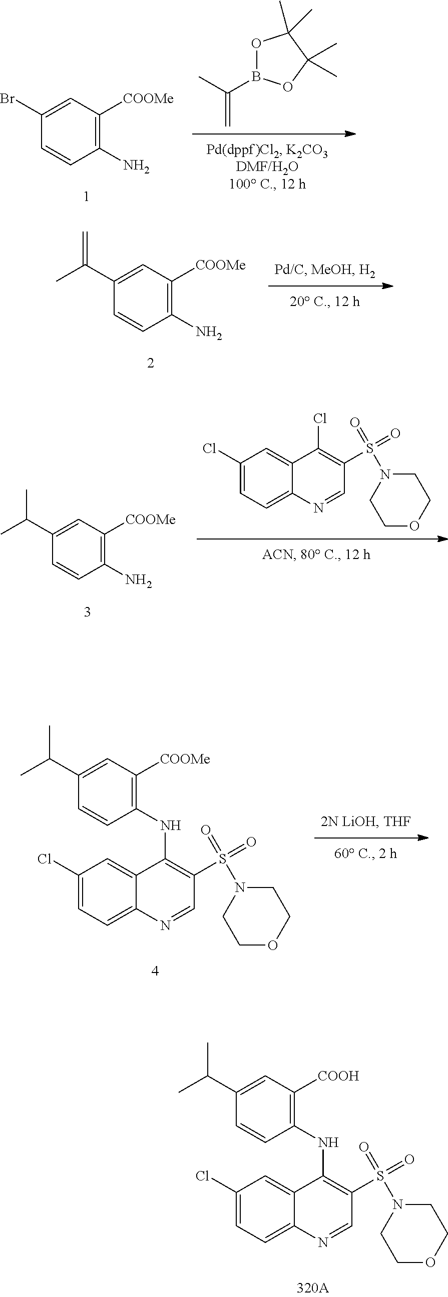

Step 1. Synthesis of methyl 2-amino-5-isopropenyl-benzoate (2): To a stirred solution of methyl 2-amino-5-bromo-benzoate (1 g, 4.35 mmol, 1 eq) in DMF (10 mL) and H₂O (2 mL) was added 2-isopropenyl-4,4,5,5-tetramethyl-1,3,2-dioxaborolane (876.51 mg, 5.22 mmol, 1.2 eq) Pd(dppf)Cl₂ (318.05 mg, 434.67 umol, 0.1 eq) and K₂CO₃ (1.80 g, 13.04 mmol, 3 eq) at 25° C., then the mixture was bubbled with N₂ for 3 times, and stirred at 100° C. for 3 h. LCMS showed the starting material was consumed completely and desired product was detected. The reaction mixture was poured into water (50 mL). The aqueous phase was extracted with ethyl acetate (50 mL*3). The combined organic phase was dried with anhydrous Na₂SO₄, filtered and concentrated in vacuum. The crude product was purified by flash column (ISCO 40 g silica, 15-20% ethyl acetate in petroleum ether, gradient over 20 min). Based on TLC (Petroleum ether:Ethyl acetate=3/1, R_{f}=0.50). Compound methyl 2-amino-5-isopropenyl-benzoate (400 mg, 2.09 mmol, 48.12% yield) was obtained as a yellow solid. MS (M+H)⁺=192.0. ¹H NMR (400 MHz, CHLOROFORM-d) 6=7.97 (d, J=2.3 Hz, 1H), 7.47 (dd, J=2.4, 8.6 Hz, 1H), 6.64 (d, J=8.6 Hz, 1H), 5.75 (br s, 2H), 4.96 (t, J=1.4 Hz, 1H), 3.89 (s, 3H), 2.13 (s, 3H).

Step 2. Synthesis of methyl 2-amino-5-isopropyl-benzoate (3): To a stirred solution of methyl 2-amino-5-isopropenyl-benzoate (100 mg, 522.94 umol, 1 eq) in MeOH (2 mL) was added Pd/C (100 mg, 10% purity) at 25° C., then the mixture was purged with H₂ for 3 times, and stirred at 25° C. for 12 h under H₂(15 psi). LCMS showed the starting material was consumed completely and desired Ms was detected. The reaction mixture was filtered, and filtrate was concentrate in vacuum. Compound methyl 2-amino-5-isopropyl-benzoate (100 mg, 517.49 umol, 98.96% yield) was obtained as a yellow oil. MS (M+H)⁺=194.2.

Step 3. Synthesis of methyl methyl 2-[(6-chloro-3-morpholinosulfonyl-4-quinolyl)amino]-5-isopropyl-benzoate (4): A solution of methyl 2-amino-5-isopropyl-benzoate (30 mg, 155.25 umol, 1 eq) and 4-[(4,6-dichloro-3-quinolyl)sulfonyl]morpholine (53.90 mg, 1 55.25 umol, 1 eq) in ACN (1 mL) was stirred at 80° C. for 12 h. LCMS showed the starting material was consumed completely and desired product was detected. The reaction mixture was concentrate in vacuum. Compound methyl 2-[(6-chloro-3-morpholinosulfonyl-4-quinolyl)amino]-5-isopropyl-benzoate (80 mg, crude) was obtained as a yellow solid. MS (M+H)⁺=504.0.

Step 4. Synthesis of 2-[(6-chloro-3-morpholinosulfonyl-4-quinolyl)amino]-5-isopropyl-benzoic acid (320A): To a stirred solution of methyl 2-[(6-chloro-3-morpholinosulfonyl-4-quinolyl)amino]-5-isopropyl-benzoate (80 mg, 158.73 umol, 1 eq) in THF (0.3 mL) a nd MEOH (0.3 mL) was added LiOH·H₂O (2 M, 158.73 uL, 2 eq) at 25° C., then the mixture was stirred at 25° C. for 12 h. LCMS showed the starting material was consumed completely and desired product was detected. The reaction mixture was concentrate in vacuum. The residue was purified by prep-HPLC (column: Phenomenex Luna 80*30 mm*3 um; mobile phase: [water(0.04% HCl)-ACN]; B %:35%-70%, 8 min). Afford 24 mg crude product. Then the crude product was dissolved with DCM (0.5 mL), and MTBE (0.5 mL) was added, then filtered and filter cake was concentrate in vacuum to give the pure product. Compound 2-[(6-chloro-3-morpholinosulfonyl-4-quinolyl)amino]-5-isopropyl-benzoic acid (6.8 mg, 12.49 umol, 7.87% yield, 96.67% purity, HCl) was obtained as a yellow solid. ¹H NMR (400 MHz, DMSO-d6) δ ppm 10.31 (br s, 1H), 9.05 (s, 1H), 8.10 (d, J=8.88 Hz, 1H), 7.82-7.92 (m, 2H), 7.52 (d, J=2.13 Hz, 1H), 7.27 (dd, J=8.50, 2.25 Hz, 1H), 6.70 (d, J=8.38 Hz, 1H), 3.29-3.37 (m, 4H), 2.99-3.09 (m, 4H), 2.85-2.96 (m, 1H), 1.19 (d, J=7.00 Hz, 6H). MS (M+H)⁺=490.0

### Example 109—Synthesis of Compound 321A

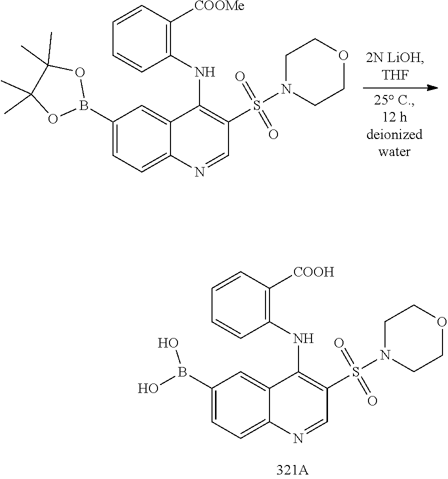 [SMILES: COC(=O)c1ccccc1Nc1c(S(=O)(=O)N2CCOCC2)cnc2ccc(B3OC(C)(C)C(C)(C)O3)cc12 ; O=C(O)c1ccccc1Nc1c(S(=O)(=O)N2CCOCC2)cnc2ccc(B(O)O)cc12]

To a stirred solution of methyl 2-[[3-morpholinosulfonyl-6-(4,4,5,5-tetramethyl-1,3,2-dioxaborolan-2-yl)-4-quinolyl]amino]benzoate (30 mg, 54.21 umol, 1 eq) in THF (2 mL) was added LiOH·H₂O (2 M, 54.21 uL, 2 eq) at 25° C., then the mixture was stirred at 25° C. for 12 h. LCMS showed the starting material was consumed completely and desired Ms was detected. The reaction mixture was adjusted by pH˜4 by adding 2N HCl. Then the mixture was concentrate in vacuum. The residue was purified by prep-HPLC (column: Phenomenex luna C 18 80*40 mm*3 um; mobile phase: [water(0.1% TFA)-ACN]; B %: 19%-30%,7 min). Compound 2-[(6-borono-3-morpholinosulfonyl-4-quinolyl)amino]benzoic acid (2.30 mg, 4.66 umol, 8.59% yield, 100% purity, HCl) was obtained as a yellow solid. ¹H NMR (400 MHz, DMSO-d6+D₂O) δ=9.03 (d, J=2.6 Hz, 1H), 8.21-8.09 (m, 2H), 8.04-7.94 (m, 2H), 7.32-7.22 (m, 1H), 7.05 (br t, J=7.7 Hz, 1H), 6.61-6.47 (m, 1H), 3.50-3.37 (m, 2H), 3.36-3.24 (m, 2H), 3.07-2.92 (m, 4H). MS (M+H)⁺=458.0

### Example 110—Synthesis of Compound 322A

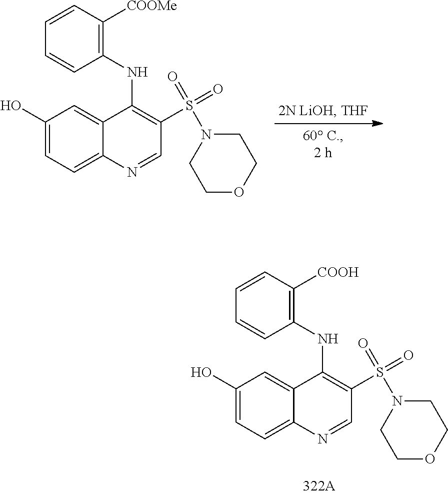 [SMILES: COC(=O)c1ccccc1Nc1c(S(=O)(=O)N2CCOCC2)cnc2ccc(O)cc12 ; O=C(O)c1ccccc1Nc1c(S(=O)(=O)N2CCOCC2)cnc2ccc(O)cc12]

To a stirred solution of methyl 2-[(6-hydroxy-3-morpholinosulfonyl-4-quinolyl)amino]benzoate (30 mg, 67.65 umol, 1 eq) in THF (2 mL) was added LiOH·H₂O (5.68 mg, 135.30 umol, 2 eq) at 25° C., then the mixture was stirred at 60° C. for 2 h. LCMS showed the starting material was consumed completely and desired Ms was detected. The residue was adjusted pH˜4 by adding 2N HCl. Then the mixture was concentrate in vacuum. The residue was purified by prep-HPLC (column: Phenomenex Luna 80*30 mm*3 um; mobile phase: [water(0.04% HCl)-ACN]; B %: 20%-45%,8 min). Compound 2-[(6-hydroxy-3-morpholinosulfonyl-4-quinolyl)amino]benzoic acid (3.50 mg, 7.51 umol, 11.10% yield, 100% purity, HCl) was obtained as a yellow solid. ¹H NMR (400 MHz, DMSO-d6) δ=10.40 (s, 2H), 8.97 (s, 1H), 8.03 (dd, J=8.4, 14.3 Hz, 2H), 7.49 (dd, J=2.5, 9.1 Hz, 1H), 7.38 (t, J=7.8 Hz, 1H), 7.09 (t, J=7.6 Hz, 1H), 6.89 (d, J=2.5 Hz, 1H), 6.65 (br d, J=8.4 Hz, 1H), 3.50-3.46 (m, 2H), 3.38-3.32 (m, 2H), 3.08-3.01 (m, 4H). MS (M+H)⁺=430.0

### Example 111—Synthesis of 323A

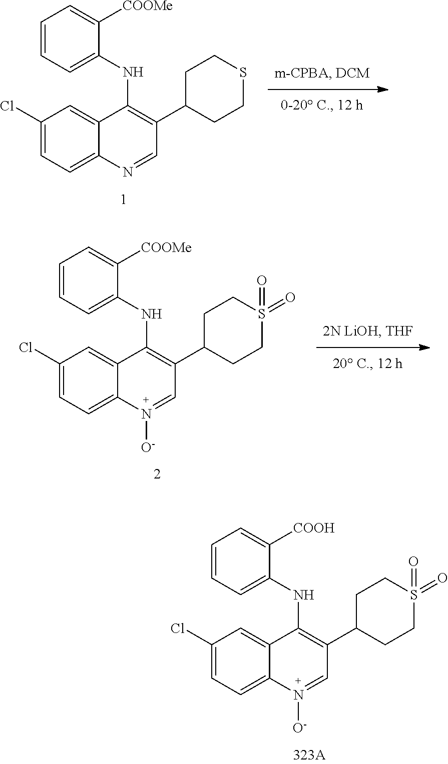

Step 1. Synthesis of methyl 2-[[6-chloro-3-(1,1-dioxothian-4-yl)-1-oxido-quinolin-1-ium-4-yl]amino]benzoate (2): To a stirred solution of methyl 2-[(6-chloro-3-tetrahydrothiopyran-4-vl-4-quinolyl)amino]benzoate (25 mg, 60.54 umol, 1 eq) in DCM (2 mL) was added m-CPBA (36.87 mg, 181.63 umol, 85% purity, 3 eq) at 0° C., then the mixture was stirred at 25° C. for 12 h. LCMS showed the starting material was consumed completely and 20% desired Ms was detected. The reaction mixture was poured into sat. Na₂SO₃. Then the mixture was concentrate in vacuum. The residue was purified by prep-HPLC (column: Phenomenex luna C18 80*40 mm*3 um; mobile phase: [water(0.04% HCl)-ACN]; B %: 29%-49%,7 min). Compound methyl 2-[[6-chloro-3-(1,1-dioxothian-4-yl)-1-oxido-quinolin-1-ium-4-yl]amino]benzoate (10 mg, 21.70 umol, 35.83% yield) was obtained as a yellow solid. MS (M+H)⁺=461.0.

Step 2. Synthesis of 2-[[6-chloro-3-(1,1-dioxothian-4-yl)-1-oxido-quinolin-1-ium-4-yl]amino]benzoic acid (323A): To a stirred solution of methyl 2-[[6-chloro-3-(1,1-dioxothian-4-yl)-1-oxido-quinolin-1-ium-4-yl]amino]benzoate (5 mg, 10.85 umol, 1 eq) in THF (0.1 mL) and MeOH (0.1 mL) was added LiOH·H₂O (2 M, 10.85 uL, 2 eq) at 25° C., then the mixture was stirred at 25° C. for 12 h. LCMS showed the starting material was consumed completely and desired Ms was detected. The reaction mixture was filtered, and filtrate was purified by prep-HPLC (column: Phenomenex Luna 80*30 mm*3 um; mobile phase: [water(0.04% HCl)-ACN]; B %: 35%-60%,8 min). Compound 2-[[6-chloro-3-(1,1-dioxothian-4-yl)-1-oxido-quinolin-1-ium-4-yl]amino]benzoic acid (1.0 mg, 1.88 umol, 17.35% yield, 90.95% purity, HCl) was obtained as a yellow solid. ¹H NMR (400 MHz, DMSO-d6+D₂O) δ ppm 8.63 (s, 1H), 8.54 (d, J=9.26 Hz, 1H), 7.94 (dd, J=7.94, 1.56 Hz, 1H), 7.82 (dd, J=9.19, 2.19 Hz, 1H), 7.66 (d, J=2.13 Hz, 1H), 7.17-7.27 (m, 1H), 6.79 (t, J=7.57 Hz, 1H), 6.18 (d, J=8.00 Hz, 1H), 3.19-3.33 (m, 2H), 3.02-3.18 (m, 3H), 2.34-2.44 (m, 1H), 1.99-2.20 (m, 3H). MS (M+H)⁺=447.0.

### Example 112—Synthesis of 324A

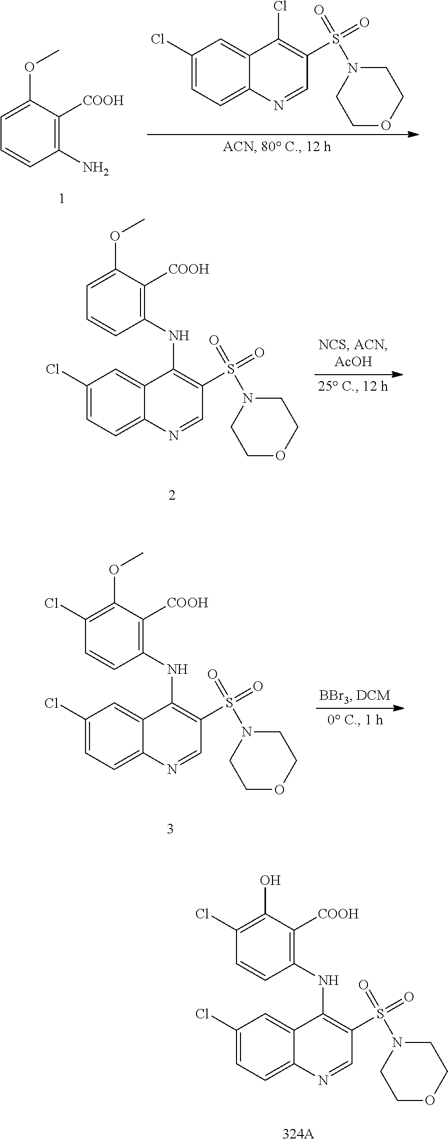 [SMILES: COc1cccc(N)c1C(=O)O ; O=S(=O)(c1cnc2ccc(Cl)cc2c1Cl)N1CCOCC1 ; COc1cccc(Nc2c(S(=O)(=O)N3CCOCC3)cnc3ccc(Cl)cc23)c1C(=O)O ; COc1c(Cl)ccc(Nc2c(S(=O)(=O)N3CCOCC3)cnc3ccc(Cl)cc23)c1C(=O)O ; O=C(O)c1c(Nc2c(S(=O)(=O)N3CCOCC3)cnc3ccc(Cl)cc23)ccc(Cl)c1O]

Step 1. Synthesis of 2-[(6-chloro-3-morpholinosulfonyl-4-quinolyl)amino]-6-methoxy-benzoic acid (2): To a solution of 4-[(4,6-dichloro-3-quinolyl)sulfonyl]morpholine (130 mg, 374.41 umol, 1 eq) in ACN (3 mL) was added 2-amino-6-methoxy-benzoic acid (62.59 mg, 374.41 umol, 1 eq), the reaction was stirred at 80° C. for 12 h. LCMS showed starting material was consumed completely and the MS of desired product was detected. The reaction mixture was concentrated in vacuum. Compound 2-[(6-chloro-3-morpholinosulfonyl-4-quinolyl)amino]-6-methoxy-benzoic acid (175 mg, 366.17 umol, 97.80% yield) was obtained as a yellow solid. MS (M+H)⁺=478.0.

Step 2. Synthesis of 3-chloro-6-[(6-chloro-3-morpholinosulfonyl-4-quinolyl)amino]-2-methoxy-benzoic acid (3): To a solution of 2-[(6-chloro-3-morpholinosulfonyl-4-quinolyl)amino]-6-methoxy-benzoic acid (165 mg, 345.25 umol, 1 eq) in AcOH (1 mL) and ACN (1 mL) was added NCS (69.15 mg, 517.87 umol, 1.5 eq, the reaction was stirred at 25° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water (0.04% HCl)-ACN]; B %: 35%-60%, 8 min) Compound 3-chloro-6-[(6-chloro-3-morpholinosulfonyl-4-quinolyl)amino]-2-methoxy-benzoic acid (30 mg, 54.66 umol, 15.83% yield, HCl) was obtained as a yellow solid. MS (M+H)⁺=512.0

Step 3. Synthesis of 3-chloro-6-[(6-chloro-3-morpholinosulfonyl-4-quinolyl)amino]-2-hydroxy-benzoic acid (324A): To a solution of 3-chloro-6-[(6-chloro-3-morpholinosulfonyl-4-quinolyl)amino]-2-methoxy-benzoic acid (25 mg, 48.79 umol, 1 eq) in DCM (1 mL) was added BBr₃ (61.12 mg, 243.97 umol, 23.51 uL, 5 eq) at 0° C., the reaction was stirred at 0° C. for 1 h under N₂. LCMS showed starting material was consumed completely and the MS of desired product was detected. The reaction mixture was concentrated in vacuum. The crude product was purified by prep-HPLC (column: Phenomenex Luna C18 75*30 mm*3 um; mobile phase: [water(FA)-ACN]; B %: 15%-65%,8 min), afford crude product 25 mg, the crude product was purified by prep-HPLC (column: Phenomenex C18 75*30 mm*3 um; mobile phase: [water(10 mmol NH₄HCO₃)-ACN]; B %: 20%-50%,8 min) Compound 3-chloro-6-[(6-chloro-3-morpholinosulfonyl-4-quinolyl)amino]-2-hydroxy-benzoic acid (7.60 mg, 15.25 umol, 31.26% yield, 100% purity) was obtained as a yellow solid. ¹H NMR (400 MHz, DMSO-d6+D₂O) δ=9.01 (s, 1H), 8.04 (d, J=9.0 Hz, 1H), 7.83 (dd, J=2.4, 9.0 Hz, 1H), 7.63 (d, J=2.4 Hz, 1H), 7.06 (d, J=8.8 Hz, 1H), 5.81 (d, J=8.8 Hz, 1H), 3.44 (br dd, J=3.1, 6.2 Hz, 2H), 3.39-3.34 (m, 2H), 3.11-3.03 (m, 2H), 3.03-2.94 (m, 2H). MS (M+H)⁺=498.1

### Example 113—Synthesis of Compound 328A

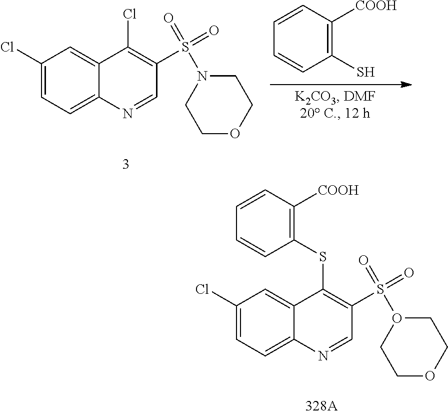

To a stirred solution of 4-[(4,6-dichloro-3-quinolyl)sulfonyl]morpholine (100 mg, 288.00 umol, 1 eq) in DMF (3 mL) was added 2-sulfanylbenzoic acid (48.85 mg, 316.81 umol, 1.1 eq) and K₂CO₃ (79.61 mg, 576.01 umol, 2 eq) at 25° C., then the mixture was stirred at 25° C. for 12 h. LCMS showed the starting material was consumed completely and desired product was detected. The reaction mixture was filtered, and filtrate was purified by prep-HPLC (column: Phenomenex luna C18 80*40 mm*3 um; mobile phase: [water(0.04% HCl)-ACN]; B %: 35%-50%,7 min). Compound 2-[(6-chloro-3-morpholinosulfonyl-4-quinolyl)sulfanyl]benzoic acid (8.3 mg, 16.15 umol, 5.61% yield, 97.54% purity, HCl) was obtained as a yellow solid. 1H NMR (400 MHz, DMSO-d6) δ=9.45 (s, 1H), 8.26 (d, J=9.0 Hz, 1H), 8.14 (d, J=2.3 Hz, 1H), 8.05 (dd, J=1.9, 7.4 Hz, 1H), 7.99 (dd, J=2.3, 9.0 Hz, 1H), 7.32-7.20 (m, 2H), 6.40 (d, J=7.6 Hz, 1H), 3.46 (br d, J=4.6 Hz, 4H), 3.28 (br d, J=4.5 Hz, 4H). MS (M+H)⁺=465.0

### Example 114—Synthesis of 330A

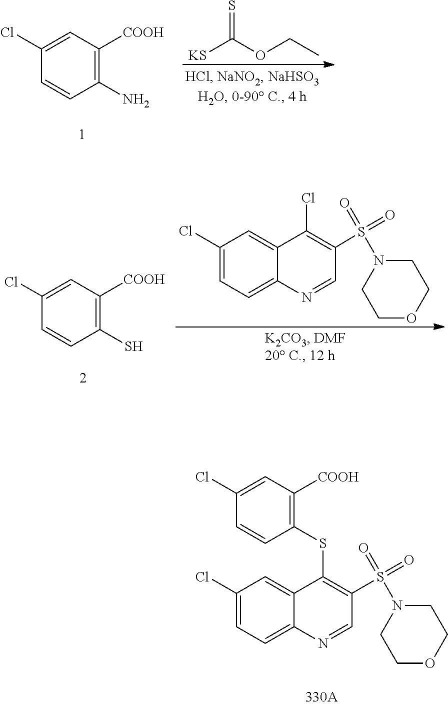

Step 1. Synthesis of 5-chloro-2-sulfanyl-benzoic acid (2): To a stirred solution of 2-amino-5-chloro-benzoic acid (2 g, 11.66 mmol, 1 eq), NaOH (2 M, 5.83 mL, 1 eq) NaNO₂ (804.23 mg, 11.66 mmol, 1 eq) in H₂O (20 mL) was added dropwise HCl (12 M, 2.91 mL, 3 eq) at 0° C. Then the mixture was stirred at 0° C. for 0.5 h. And the mixture was adjusted pH˜7 by adding AcOK (3.78 g, 38.47 mmol, 3.3 eq).ethoxycarbothioylsulfanylpotassium (5.61 g, 34.97 mmol, 3 eq) was added, and the mixture was stirred at 90° C. for 1 h. Then cooled to 0° C., the mixture was adjusted pH˜4 by adding HCl (12 M, 1.94 mL, 2 eq). The reaction mixture was basified with NaOH (466.22 mg, 11.66 mmol, 1 eq) and heated to 85° C. for 2 h. To this mixture was added portion wise NaHSO₃ (1.21 g, 11.66 mmol, 819.57 uL, 1 eq) and the mixture was heated to 85° C. for 30 min. LCMS showed the starting material was consumed completely and desired product was detected. The mixture was filtered, filtrate was cooled to 0° C. and acidified with conc. HCl (20 mL). The filtrate was purified by prep-HPLC (column: Phenomenex luna C18 100*40 mm*5 um; mobile phase: [water(0.1% TFA)-ACN]; B %: 5%-50%,8 min). Compound 5-chloro-2-sulfanyl-benzoic acid (200 mg, 660.82 umol, 5.67% yield, TFA) was obtained as a yellow solid. MS (M−H)⁻=187.0.

Step 2. Synthesis of 5-chloro-2-[(6-chloro-3-morpholinosulfonyl-4-quinolyl)sulfanyl]benzoic acid (330A): To a stirred solution of 5-chloro-2-sulfanyl-benzoic acid (27.16 mg, 144.00 umol, 1 eq) in DMF (0.5 mL) was added 4-[(4,6-dichloro-3-quinolyl)sulfonyl]morpholine (50 mg, 144.00 umol, 1 eq) and K₂CO₃ (39.80 mg, 288.00 umol, 2 eq) at 25° C., then the mixture was stirred at 25° C. for 12 h. LCMS showed the starting material was consumed completely and desired product was detected. The reaction mixture was filtered, and filtrate was purified directly. The filtrate was purified by prep-HPLC (column: Phenomenex Luna 80*30 mm*3 um; mobile phase: [water(0.1% TFA)-ACN]; B %: 45%-70%,8 min). Compound 5-chloro-2-[(6-chloro-3-morpholinosulfonyl-4-quinolyl)sulfanyl]benzoic acid (34.30 mg, 55.25 umol, 38.36% yield, 98.80% purity, TFA) was obtained as a yellow solid. ¹H NMR (400 MHz, DMSO-d6) δ ppm 9.44 (s, 1H), 8.26 (d, J=9.01 Hz, 1H), 8.17 (d, J=2.25 Hz, 1H), 7.89-8.06 (m, 2H), 7.28 (dd, J=8.69, 2.56 Hz, 1H), 6.41 (d, J=8.76 Hz, 1H), 3.47-3.50 (m, 4H), 3.23-3.30 (m, 4H). MS (M+H)⁺=498.9.

### Example 115—Synthesis of Compound 338A

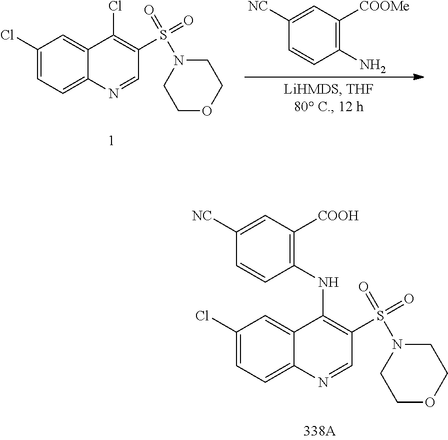 [SMILES: O=S(=O)(c1cnc2ccc(Cl)cc2c1Cl)N1CCOCC1 ; COC(=O)c1cc(C#N)ccc1N ; N#Cc1ccc(Nc2c(S(=O)(=O)N3CCOCC3)cnc3ccc(Cl)cc23)c(C(=O)O)c1]

To a stirred solution of methyl 2-amino-5-cyano-benzoate (50.74 mg, 288.00 umol, 2 eq) in THF (2 mL) was added and LIHMDS (1 M, 288.00 uL, 2 eq) at 25° C., the mixture was stirred at 25° C. for 0.5 h. Then 4-[(4,6-dichloro-3-quinolyl)sulfonyl]morpholine (50 mg, 144.00 umol, 1 eq) was added, and the mixture stirred at 80° C. for 12 h. LCMS showed the starting material was consumed completely and desired product was detected. The reaction mixture was poured into sat. NH₄Cl (5 mL). The aqueous phase was extracted with ethyl acetate (10 mL*2). The combined organic phase was dried with anhydrous Na₂SO₄, filtered and concentrated in vacuum. The residue was purified by prep-HPLC (column: Phenomenex Luna 80*30 nm*3 um; mobile phase: [water(0.04% HCl)-ACN]; B %: 35%-60%,8 min). Compound 2-[(6-chloro-3-morpholinosulfonyl-4-quinolyl)amino]-5-cyano-benzoic acid (21.8 mg, 41.46 umol, 28.79% yield, 96.88% purity, HCl) was obtained as a yellow solid. ¹H NMR (400 MHz, DMSO-d6) δ ppm 10.71-10.91 (m, 1H), 9.18 (s, 1H), 8.34 (d, J=2.00 Hz, 1H), 8.20 (d, J=9.01 Hz, 1H), 7.96 (dd, J=9.01, 2.25 Hz, 1H), 7.74 (d, J=2.25 Hz, 1H), 7.70 (dd, J=8.76, 2.00 Hz, 1H), 6.65 (d, J=8.75 Hz, 1H), 3.44-3.51 (m, 2H), 3.29-3.39 (m, 2H), 3.02 (t, J=4.50 Hz, 4H). MS (M+H)⁺=473.1

### Example 115A—Synthesis of 339A

Synthetic scheme is shown in FIG. 39B.

### 6-chloro-3-iodo-1H-quinolin-4-one (2)

To a solution of 6-chloroquinolin-4-ol (25 g, 139.20 mmol, 1 eq) in the solution of MeCN (250 mL) and AcOH (33 mL) was added NIS (31.32 g, 139.20 mmol, 1 eq), the mixture was stirred at 25° C. for 12 hr. LCMS showed the reaction was complete. The reaction mixture was filtered and the filter cake was concentrated in vacuo. Compound 6-chloro-3-iodo-1H-quinolin-4-one (40 g, crude) was obtained as a white solid. MS (M+H)⁺=305.9.

### 4-bromo-6-chloro-3-iodo-quinoline (3)

To a solution of 6-chloro-3-iodo-1H-quinolin-4-one (20 g, 65.47 mmol, 1 eq) in DMF (200 mL) was added POBr3 (22.52 g, 78.56 mmol, 7.99 mL, 1.2 eq) in batches at 0° C., the mixture was stirred at 70° C. for 3 hr. LCMS showed the reaction was complete. The reaction was cooled to ambient temperature, poured into ice water slowly. Then the mixture was filtered and the filter cake was concentrated in vacuo. Compound methyl 4-bromo-6-chloro-3-iodo-quinoline (23 g, crude) was obtained as a white solid. MS (M+H)⁺=367.8.

### 4-(4-bromo-6-chloro-3-quinolyl)morpholine (4)

To a solution of 4-bromo-6-chloro-3-iodo-quinoline (1 g, 2.71 mmol, 1 eq) in toluene (10 mL) was added morpholine (236.48 mg, 2.71 mmol, 238.87 uL, 1 eq), t-BuONa (782.58 mg, 8.14 mmol, 3 eq), rac-BINAP-Pd-G3 (269.38 mg, 271.45 umol, 0.1 eq), BINAP (169.02 mg, 271.45 umol, 0.1 eq), the mixture was stirred at 100° C. for 12 hr under N₂. 20 mL of Water was added to the reaction, the reaction mixture was extracted with EtOAc (30 mL*2). The combined organic layers were washed with brine (20 mL), dried over Na₂SO₄ and filtered. The filtrate was concentrated to dryness to give a residue. The crude product was purified by flash column (ISCO 40 g silica, 5-30% ethyl acetate in petroleum ether, gradient over 20 min). Compound 4-(4-bromo-6-chloro-3-quinolyl)morpholine (1.5 g, 4.58 mmol, 56.23% yield) was obtained as a gray solid. MS (M+H)¹=326.9.

### Methyl 5-chloro-2-[(6-chloro-3-morpholino-4-quinolyl)amino]benzoate (5A)

To a solution of 4-(4-bromo-6-chloro-3-quinolyl)morpholine (2.5 g, 7.63 mmol, 1 eq) in t-Amyl Alcohol (30 mL) was added methyl 2-amino-5-chloro-benzoate (1.42 g, 7.63 mmol, 1 eq), Cs₂CO₃ (4.97 g, 15.26 mmol, 2 eq), RuPhos Pd G3 (638.24 mg, 763.12 umol, 0.1 eq), the mixture was stirred at 90° C. for 12 hr under N₂. LCMS showed the starting material was consume completely and desired product was detected. 50 mL of Water was added to the reaction, the reaction mixture was extracted with ethyl acetate (30 mL*2). The combined organic layers were washed with brine (30 mL), dried over Na₂SO₄ and filtered. The filtrate was concentrated to dryness to give residue. The crude product was purified by flash column (ISCO 40 g silica, 5-40% ethyl acetate in petroleum ether, gradient over 20 min). The crude product was purified by Prep-HPLC (column: Welch Xtimate C18 250*70 mm #10 um; mobile phase: [water(NH₄HCO₃)-ACN]; B %: 20%-50%,20 min). methyl 5-chloro-2-[(6-chloro-3-morpholino-4-quinolyl)amino]benzoate (340 mg, 786.49 umol, 10.31% yield) was obtained as a yellow solid. MS (M+H)⁺=432.1.

### 5-chloro-2-[(6-chloro-3-morpholino-4-quinolyl)amino]benzoic acid (339A)

To a solution of methyl 5-chloro-2-[(6-chloro-3-morpholino-4-quinolyl)amino]benzoate (300 mg, 693.96 umol, 1 eq) in THF (3 mL) and MeOH (1 mL) was added LiOH·H₂O (2 M, 1.11 mL, 3.20 eq), the mixture was stirred at 50° C. for 1 hr. LCMS showed the reaction was complete. The mixture was acidified by adding hydrochloric acid (1 M, 2 mL) dropwise to pH=5-6. 5 mL of Water was added to the reaction, the reaction mixture was extracted with ethyl acetate (15 mL*2). The combined organic layers were washed with brine (5 mL), dried over Na₂SO₄ and filtered. The filtrate was concentrated to dryness to give residue. The crude product was purified by Prep-HPLC (column: Waters Xbridge BEH C18 100*30 mm*10 um; mobile phase: [water(NH₄HCO₃)-ACN]; B %: 25%-55%,8 min). Compound 5-chloro-2-[(6-chloro-3-morpholino-4-quinolyl)amino]benzoic acid (170 mg, 398.06 umol, 57.36% yield) was obtained as a yellow solid. ¹H NMR (400 MHz, DMSO-d₆) δ 11.43-11.04 (m, 1H), 8.75 (s, 1H), 7.97 (d, J=9.0 Hz, 1H), 7.87 (d, J=2.6 Hz, 1H), 7.85 (d, J=2.3 Hz, 1H), 7.64-7.58 (m, 1H), 7.28-7.22 (m, 1H), 7.21-7.07 (m, 1H), 6.44-6.37 (m, 1H), 3.44-3.38 (m, 4H), 3.03 (br s, 4H). MS (M+H)⁺=418.1

### Example 115B—Synthesis of 339A

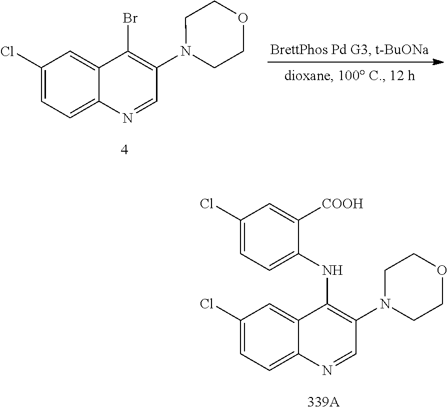 [SMILES: Clc1ccc2ncc(N3CCOCC3)c(Br)c2c1 ; O=C(O)c1cc(Cl)ccc1Nc1c(N2CCOCC2)cnc2ccc(Cl)cc12]

### Synthesis of 5-chloro-2-[(6-chloro-3-morpholino-4-quinolyl)amino]benzoic acid (339A

To a stirred solution of 4-(4-bromo-6-chloro-3-quinolyl)morpholine (50 mg, 152.62 umol, 1 eq) in dioxane (2 mL) was added methyl 2-amino-5-chloro-benzoate (31.16 mg, 167.88 umol, 1.1 eq) BrettPhos Pd G3 (13.84 mg, 15.26 umol, 0.1 eq), BRETTPHOS (8.19 mg, 15.26 umol, 0.1 eq) and t-BuONa (44.00 mg, 457.86 umol, 3 eq), the mixture was purged with N₂ for 3 times, and stirred at 100° C. for 12 h. LCMS showed the starting material was consumed completely and 20% desired product was detected. The reaction mixture was concentrate in vacuum. The residue was purified by prep-HPLC (column: Phenomenex Luna 80*30 mm*3 um; mobile phase: [water(0.1% TFA)-ACN]; B %: 10%-40%,8 min). Afford 10 mg crude product. The crude product was purified by prep-HPLC (column: Waters Xbridge BEH C18 100*30 mm*10 um; mobile phase: [water(NH₄HCO₃)-ACN]; B %: 25%-55%,10 min). Compound 5-chloro-2-[(6-chloro-3-morpholino-4-quinolyl)amino]benzoic acid (3.8 mg, 9.05 umol, 5.93% yield, 99.58% purity) was obtained as a yellow solid. ¹H NMR (400 MHz, DMSO-d6) δ ppm 10.60-10.90 (m, 1H), 8.77 (s, 1H), 7.99 (d, J=8.88 Hz, 1H), 7.77-7.92 (m, 2H), 7.62 (dd, J=8.88, 1.50 Hz, 1H), 7.29 (br d, J=8.75 Hz, 1H), 7.01-7.23 (m, 1H), 6.43 (d, J=8.76 Hz, 1H), 3.41 (br s, 4H), 3.04 (br s, 4H). MS (M+H)⁺=418.0.

### Example 115C—Synthesis of 340A

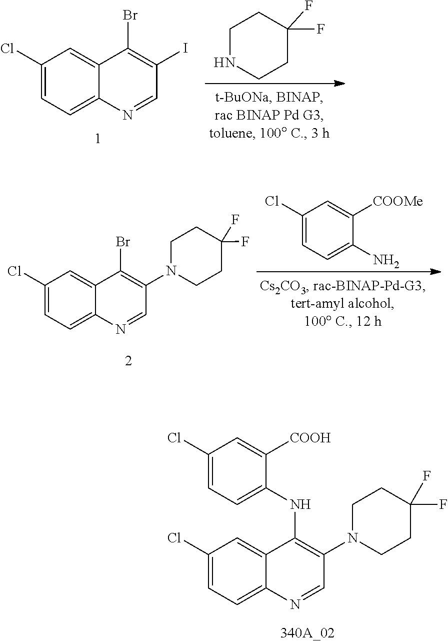

### Synthesis of 4-bromo-6-chloro-3-(4,4-difluoro-1-piperidyl)quinoline (1)

A mixture of 4-bromo-6-chloro-3-iodo-quinoline (10 g, 27.14 mmol, 1 eq), 4,4-difluoropiperidine (3.29 g, 27.14 mmol, 1 eq), t-BuONa (7.83 g, 81.43 mmol, 3 eq), BINAP (1.69 g, 2.71 mmol, 0.1 eq) and rac-BINAP-Pd-G3 (2.69 g, 2.71 mmol, 0.1 eq) in toluene (130 mL) was degassed and purged with N₂ for 3 times, and then the mixture was stirred at 100° C. for 12 h under N₂ atmosphere. LCMS showed the starting material was consumed completely and desired MS was detected. The reaction mixture was filtered. 100 mL of water was added to the filtrate and extracted with Ethyl acetate (100 mL*3). The combined organic layers were washed with brine (80 mL), dried over Na₂SO₄ and concentrated to dryness to give a residue. The crude product was purified by flash column (ISCO 120 g silica, 0-32% ethyl acetate in petroleum ether, gradient over 60 min). Compound 4-bromo-6-chloro-3-(4,4-difluoro-1-piperidyl)quinoline (6.4 g, 17.70 mmol, 65.20% yield) was obtained as a white solid. ¹H NMR (400 MHz, CHLOROFORM-d) δ 8.69 (s, 1H), 8.21 (d, J=2.0 Hz, 1H), 8.01 (d, J=8.4 Hz, 1H), 7.60 (dd, J=2.4, 9.0 Hz, 1H), 3.40-3.36 (m, 4H), 2.26 (tt, J=5.8, 13.7 Hz, 4H). MS (M+H)⁺=361.0

### Synthesis of 5-chloro-2-[[6-chloro-3-(4,4-difluoro-1-piperidyl)-4-quinolyl]amino]benzoic acid (340A)

Two batches: A mixture of 4-bromo-6-chloro-3-(4,4-difluoro-1-piperidyl)quinoline (2 g, 5.53 mmol, 1 eq), methyl 2-amino-5-chloro-benzoate (1.03 g, 5.53 mmol, 1 eq), Cs₂CO₃ (3.60 g, 11.06 mmol, 2 eq), rac-BINAP-Pd-G3 (548.88 mg, 553.08 umol, 0.1 eq) in tert-amyl alcohol (30 mL) was degassed and purged with N₂ for 3 times, and then the mixture was stirred at 100° C. for 12 h under N₂ atmosphere. LCMS showed the starting material was consumed completely and desired MS was detected. The two batched were for work-up. The reaction mixture was filtered. 40 mL of water was added to the filtrate and extracted with Ethyl acetate (40 mL*3). The combined organic layers were washed with brine (30 mL) dried over Na₂SO₄ and concentrated to dryness to give residue. The crude product was purified by flash column (ISCO 80 g silica, 0-64% ethyl acetate in petroleum ether, gradient over 60 min). Compound 5-chloro-2-[[6-chloro-3-(4,4-difluoro-1-piperidyl)-4-quinolyl]amino]benzoic acid (1.3 g, 2.85 mmol, 51.55% yield) was obtained as a yellow solid. ¹H NMR (400 MHz, DMSO-d6) δ 9.89 (br s, 1H), 8.84 (s, 1H), 8.01 (d, J=8.9 Hz, 1H), 7.89 (d, J=2.6 Hz, 1H), 7.80 (d, J=2.3 Hz, 1H), 7.65 (dd, J=2.3, 8.9 Hz, 1H), 7.38 (dd, J=2.7, 8.9 Hz, 1H), 6.51 (d, J=9.0 Hz, 1H), 3.13-3.06 (m, 4H), 1.80-1.70 (m, 4H). MS (M+H)⁺=452.1.

### Example 115D—Synthesis of 340A

Synthetic scheme is shown in FIG. 39C.

### Synthesis of 6-chloro-3-iodo-1H-quinolin-4-one (2)

To a solution of 6-chloroquinolin-4-ol (10 g, 55.68 mmol, 1 eq) in ACN (150 mL) and AcOH (20 mL) was added NIS (12.53 g, 55.68 mmol, 1 eq), the reaction was stirred at 20° C. for 2 h. LCMS showed starting material was consumed completely and the MS of desired product was detected. The reaction mixture was filtered, the filter caked was concentrated in vacuum. Compound 6-chloro-3-iodo-1H-quinolin-4-one (14 g, 45.83 mmol, 82.310% yield) was obtained as a yellow solid. 2. MS (M+H)⁺=305.8.

### Synthesis of 4-bromo-6-chloro-3-iodo-quinoline (3)

To a solution of 6-chloro-3-iodo-1H-quinolin-4-one (10.00 g, 32.73 mmol, 1 eq) in DMF (80 mL) was added POBr₃ (11.26 g, 39.28 mmol, 3.99 mL, 1.2 eq) at 0° C., the reaction was stirred at 70° C. for 3 h. LCMS showed starting material was consumed completely and the MS of desired product was detected. The reaction was cooled to ambient temperature, quenched with water (80 ml) and extracted with ethyl acetate (80 ml). The organic layer was washed with water brine, dried over anhydrous sodium sulfate, filtered and concentrated in vacuum. The residue was purified by flash column (ISCO 40 g silica, 10-30% ethyl acetate in petroleum ether, gradient over 20 min). TLC (Petroleum ether/Ethyl acetate=3:1, R_{f}=0.54) Compound 4-bromo-6-chloro-3-iodo-quinoline (1.8 g, 4.89 mmol, 14.93% yield) was obtained as a white solid. 5. MS (M+H)⁺=369.8.

### Synthesis of 4-bromo-6-chloro-3-(4,4-difluoro-1-piperidyl) quinoline (4)

To a solution of 4-bromo-6-chloro-3-iodo-quinoline (400 mg, 1.09 mmol, 1 eq) in toluene (4 mL) was added NaOBu-t (313.04 mg, 3.26 mmol, 3 eq), BINAP (67.61 mg, 108.58 umol, 0.1 eq), [2-(2-aminophenyl)phenyl]-methylsulfonyloxy-palladium; [1-(2-diphenylphosphanyl-1-naphthyl)-2-naphthyl]-diphenyl-phosphane (107.75 mg, 108.58 umol, 0.1 eq) and 4,4-difluoropiperidine (170.98 mg, 1.41 mmol, 1.3 eq), the reaction was stirred at 100° C. for 12 h under Ar. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water (0.04% HCl)-ACN]; B %: 45%-75%, 8 min) Compound 4-bromo-6-chloro-3-(4,4-difluoro-1-piperidyl) quinoline (80 mg, 221.23 umol, 20.38% yield) was obtained as a white solid. 7. MS (M+H)⁺=363.0.

### Synthesis of 5-chloro-2-[[6-chloro-3-(4,4-difluoro-1-piperidyl)-4-quinolyl]amino]benzoic acid (340A)

To a solution of 4-bromo-6-chloro-3-(4,4-difluoro-1-piperidyl)quinoline (40 mg, 110.62 umol, 1 eq) in toluene (2 mL) was added NaOBu-t (31.89 mg, 331.85 umol, 3 eq), RuPhos (5.16 mg, 11.06 umol, 0.1 eq) and RuPhos Pd G3 (9.25 mg, 11.06 umol, 0.1 eq) and methyl 2-amino-5-chloro-benzoate (24.64 mg, 132.74 umol, 1.2 eq) the reaction was stirred at 100° C. for 12 h under Ar. LCMS showed starting material was consumed completely and the MS of desired product was detected. The reaction mixture was filtered, the filtrate was concentrated in vacuum. The crude product was purified by prep-HPLC (column: Phenomenex Luna 80*30 mm*3 um; mobile phase: [water (0.04% HCl)-ACN]; B %: 30%-50%, 8 min). Compound 5-chloro-2-[[6-chloro-3-(4,4-difluoro-1-piperidyl)-4-quinolyl]amino]benzoic acid (8.3 mg, 17.09 umol. 15.45% yield, 93.15% purity) was obtained as a yellow solid. ¹H NMR (400 MHz, DMSO-d₆) δ=10.21 (br d, J=3.6 Hz, 1H), 8.86 (s, 1H), 8.33-8.14 (m, 1H), 8.08 (br d, J=8.8 Hz, 1H), 7.94-7.82 (m, 2H), 7.56 (br d, J=9.0 Hz, 1H), 7.14-6.89 (m, 1H), 3.04 (br s, 4H), 1.78-1.59 (m, 4H). MS (M+H)⁺=452.1.

### Example 116—Synthesis of 341A

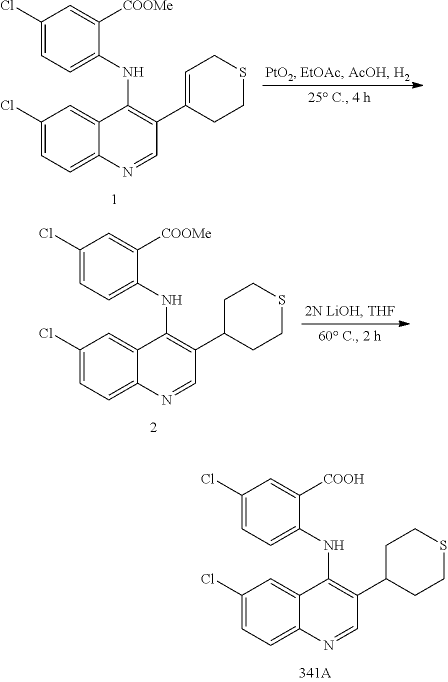 [SMILES: COC(=O)c1cc(Cl)ccc1Nc1c(C2=CCSCC2)cnc2ccc(Cl)cc12 ; COC(=O)c1cc(Cl)ccc1Nc1c(C2CCSCC2)cnc2ccc(Cl)cc12 ; O=C(O)c1cc(Cl)ccc1Nc1c(C2CCSCC2)cnc2ccc(Cl)cc12]

Step 1. Synthesis of methyl 5-chloro-2-[(6-chloro-3-tetrahydrothiopyran-4-yl-4-quinolyl)amino]benzoate (2): A solution of methyl 5-chloro-2-[[6-chloro-3-(3,6-dihydro-2H-thiopyran-4-yl)-4-quinolyl]amino]benzoate (150 mg, 336.81 umol, 1 eq) and PtO₂ (150 mg, 660.57 umol, 1.96 eq), AcOH (2.02 mg, 33.68 umol, 1.93 uL, 0.1 eq) in EtOAc (2 mL) was stirred at 25° C. for 3 h under H2(15 psi). LCMS showed 60% desired product was detected. The reaction mixture was filtered, and filtrate was concentrate in vacuum. Compound methyl 5-chloro-2-[(6-chloro-3-tetrahydrothiopyran-4-yl-4-quinolyl)amino]benzoate (170 mg, 266.00 umol, 78.98% yield, 70% purity) was obtained as a yellow oil. MS (M−H)⁻=447.1.

Step 2. Synthesis of 5-chloro-2-[(6-chloro-3-tetrahydrothiopyran-4-yl-4-quinolyl)amino]benzoic acid (341A): To a stirred solution of methyl 5-chloro-2-[(6-chloro-3-tetrahydrothiopyran-4-yl-4-quinolyl)amino]benzoate (150 mg, 335.29 umol, 1 eq) in THF (0.5 mL) and MeOH (0.5 mL) was added LiOH·H₂O (2 M, 335.29 uL, 2 eq), then the mixture was stirred at 25° C. for 3 h. LCMS showed the starting material was consumed completely and desired product was detected. The reaction mixture was adjusted pH˜5 by adding 2N HCl. The mixture was concentrate in vacuum. The residue was purified by prep-HPLC (column: Phenomenex Luna 80*30 mm*3 um; mobile phase: [water(0.04% HCl)-ACN]; B %: 25%-55%,8 min). Compound 5-chloro-2-[(6-chloro-3-tetrahydrothiopyran-4-yl-4-quinolyl)amino]benzoic acid (15.5 mg, 32.38 umol, 9.66% yield, 98.15% purity, HCl) was obtained as a yellow solid. ¹H NMR (400 MHz, DMSO-d6) δ ppm 9.99 (br s, 1H), 8.99 (s, 1H), 8.15 (d, J=8.63 Hz, 1H), 7.93 (d, J=2.63 Hz, 1H), 7.86-7.93 (m, 2H), 7.45 (dd, J=8.82, 2.31 Hz, 1H), 6.66 (br d, J=7.13 Hz, 1H), 2.86 (br t, J=11.32 Hz, 1H), 2.53-2.72 (m, 4H), 1.93-2.11 (m, 3H), 1.78-1.92 (m, 1H). MS (M+H)⁺=433.0.

### Example 117—Synthesis of 342A

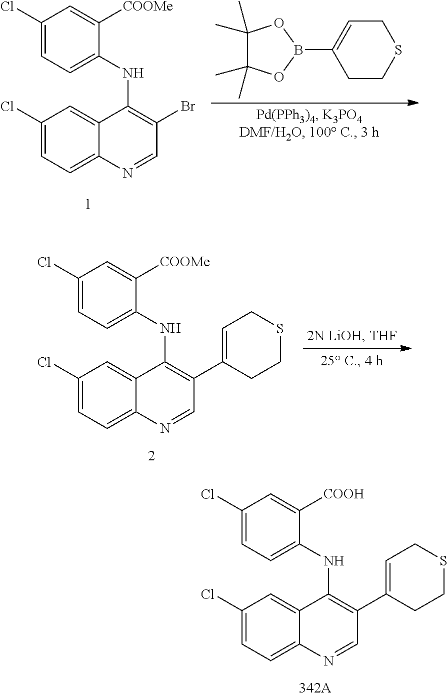

Step 1. Synthesis of methyl 5-chloro-2-[[6-chloro-3-(3,6-dihydro-2H-thiopyran-4-yl)-4-quinolyl]amino]benzoate (2): To a stirred solution of methyl 2-[(3-bromo-6-chloro-4-quinolyl)amino]-5-chloro-benzoate (2 g, 4.69 mmol, 1 eq) in DMF (10 mL) and H₂O (2 mL) was added 2-(3,6-dihydro-2H-thiopyran-4-yl)-4,4,5,5-tetramethyl-1,3,2-dioxaborolane (1.06 g, 4.69 mmol, 1 eq), Pd(PPh₃)₄ (542.40 mg, 469.38 umol, 0.1 eq) and K₃PO₄ (2.99 g, 14.08 mmol, 3 eq) at 25° C., then the mixture was purged with N₂ for 3 times, and stirred at 100° C. for 4 h. LCMS showed the starting material was consumed completely and desired product was detected. The reaction mixture was poured into water (100 mL) The aqueous phase was extracted with ethyl acetate (200 mL*2). The combined organic phase was dried with anhydrous Na₂SO₄, filtered and concentrated in vacuum. The crude product was purified by flash column (ISCO 10 g silica, 30-40% ethyl acetate in petroleum ether, gradient over 20 min). Based on TLC (Petroleum ether:Ethyl acetate=3/1, R_{f}=0.55). Compound methyl 5-chloro-2-[[6-chloro-3-(3,6-dihydro-2H-thiopyran-4-yl)-4-quinolyl]amino]benzoate (400 mg, 898.15 umol, 19.13% yield) was obtained as a yellow solid. MS (M−H)⁻=445.1.

Step 2. Synthesis of 5-chloro-2-[[6-chloro-3-(3,6-dihydro-2H-thiopyran-4-yl)-4-quinolyl]amino]benzoic acid (342A): To a stirred solution of methyl 5-chloro-2-[[6-chloro-3-(3,6-dihydro-2H-thiopyran-4-yl)-4-quinolyl]amino]benzoate (60 mg, 134.72 umol, 1 eq) in THF (0.5 mL) and MeOH (0.5 mL) was added LiOH·H₂O (2 M, 134.72 uL, 2 eq), then the mixture was stirred at 25° C. for 4 h. LCMS showed the starting material was consumed completely and desired product was detected. The reaction mixture was concentrate in vacuum. The residue was purified by prep-HPLC (column: Phenomenex Luna 80*30 mm*3 um; mobile phase: [water(0.4% HCl)-ACN]; B %: 25%-50%,8 min). Compound 5-chloro-2-[[6-chloro-3-(3,6-dihydro-2H-thiopyran-4-yl)-4-quinolyl]amino]benzoic acid (2.2 mg, 4.70 umol, 3.49% yield, 100% purity, HCl) was obtained as a yellow solid. ¹H NMR (400 MHz, DMSO-d6+D₂O) δ ppm 8.57 (s, 1H), 8.39 (d, J=1.88 Hz, 1H), 8.04 (d, J=9.01 Hz, 1H), 7.93 (dd, J=8.94, 2.19 Hz, 1H), 7.89 (d, J=2.63 Hz, 1H), 7.50 (dd, J=8.76, 2.63 Hz, 1H), 6.87 (d, J=8.63 Hz, 1H), 5.91 (br s, 1H), 3.00 (br s, 2H), 2.12-2.30 (m, 4H). MS (M+H)⁺=433.9.

### Example 117A—Synthesis of 343A

Synthetic scheme is shown in FIG. 39D.

### Synthesis of methyl 5-chloro-2-[[6-chloro-3-(4,4-difluorocyclohexen-1-yl)-4-quinolyl]amino]benzoate (2)

To a stirred solution of methyl 2-[(3-bromo-6-chloro-4-quinolyl)amino]-5-chloro-benzoate (1 g, 2.35 mmol, 1 eq) and 2-(4,4-difluorocyclohexen-1-yl)-4,4,5,5-tetramethyl-1,3,2-dioxaborolane (572.85 mg, 2.35 mmol, 1 eq) in DMF (15 mL) and H₂O (3 mL) was added Pd(PPh₃)₄ (271.20 mg, 234.69 umol, 0.1 eq) and K₃PO₄ (1.49 g, 7.04 mmol, 3 eq), the mixture was purged with N₂ for 3 times, and stirred at 100° C. for 3 h. LCMS showed the starting material was consumed completely and desired product was detected. The reaction mixture was poured into water (50 mL). The aqueous phase was extracted with ethyl acetate (50 mL*2). The combined organic phase was dried with anhydrous Na₂SO₄, filtered and concentrated in vacuum. The crude product was purified by flash column (ISCO 10 g silica, 15-20% ethyl acetate in petroleum ether, gradient over 20 min). Based on TLC (Petroleum ether:Ethyl acetate=3/1, R_{f}=0.43). Compound methyl 5-chloro-2-[[6-chloro-3-(4,4-difluorocyclohexen-1-yl)-4-quinolyl]amino]benzoate (500 mg, 1.08 mmol, 45.98% yield) was obtained as a yellow solid. 2. MS (M+H)⁺=463.1.

### Synthesis of methyl 5-chloro-2-[[6-chloro-3-(4,4-difluorocyclohexyl)-4-quinolyl]amino]benzoate (3)

A solution of methyl 5-chloro-2-[[6-chloro-3-(4,4-difluorocyclohexen-1-yl)-4-quinolyl]amino]benzoate (300 mg, 647.52 umol, 1 eq) and PtO2 (300.00 mg, 1.32 mmol, 2.04 eq) in EtOAc (5 mL) and AcOH (0.1 mL) at 25° C., the mixture was purged with H₂ for 3 times, and the mixture was stirred at 25° C. for 12 h under hydrogen balloon (15 psi). LCMS showed the starting material was consumed completely and desired product was detected. The reaction mixture was filtered, and filtrate was concentrated in vacuum. The residue was purified by prep-HPLC (column: Phenomenex Luna 80*30 mm*3 um; mobile phase: [water(TFA)-ACN]; B %: 30%-60%,8 min). Compound methyl 5-chloro-2-[[6-chloro-3-(4,4-difluorocyclohexyl)-4-quinolyl]amino]benzoate (8 mg, 17.19 umol, 2.66% yield) was obtained as a yellow solid. 4. MS (M+H)⁺=465.2.

### Synthesis of 5-chloro-2-[[6-chloro-3-(4,4-difluorocyclohexyl)-4-quinolyl]amino]benzoic acid (343A)

To a stirred solution of methyl 5-chloro-2-[[6-chloro-3-(4,4-difluorocyclohexyl)-4-quinolyl]amino]benzoate (8 mg, 17.19 umol, 1 eq) in THF (1 mL) and MeOH (0.2 mL) was added LiOH·H₂O (2 M, 17.19 uL, 2 eq) at 25° C., the mixture was stirred at 60° C. for 2 h. LCMS showed the starting material was consumed completely and desired product was detected. The reaction mixture was adjusted pH˜4 by adding 2N HCl. The mixture was concentrate in vacuum. The crude product was purified by prep-HPLC (column: Phenomenex Luna C18 150*30 mm*5 um; mobile phase: [water(0.1% TFA)-ACN]; B %: 25%-65%,8 min). Compound 5-chloro-2-[[6-chloro-3-(4,4-difluorocyclohexyl)-4-quinolyl]amino]benzoic acid (5.60 mg, 9.88 umol, 57.44% yield, 99.69% purity, TFA) was obtained as a yellow solid. ¹H NMR (400 MHz, DMSO-d6) δ ppm 9.87 (br s, 1H), 9.01 (s, 1H), 8.10 (d, J=9.01 Hz, 1H), 7.91 (d, J=2.63 Hz, 1H), 7.82 (br d, J=9.01 Hz, 1H), 7.76 (br s, 1H), 7.30-7.39 (m, 1H), 6.40 (br d, J=8.25 Hz, 1H), 3.02 (br t, J=11.69 Hz, 1H), 2.05-2.18 (m, 2H), 1.67-2.02 (m, 6H). MS (M+H)⁺=451.0.

### Example 118—Synthesis of 344A

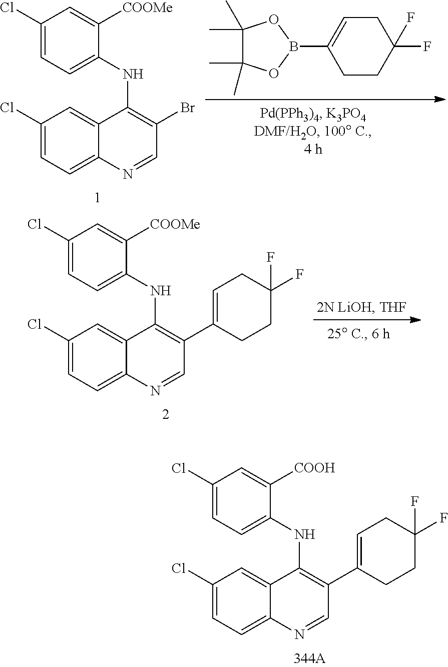 [SMILES: COC(=O)c1cc(Cl)ccc1Nc1c(Br)cnc2ccc(Cl)cc12 ; CC1(C)OB(C2=CCC(F)(F)CC2)OC1(C)C ; COC(=O)c1cc(Cl)ccc1Nc1c(C2=CCC(F)(F)CC2)cnc2ccc(Cl)cc12 ; O=C(O)c1cc(Cl)ccc1Nc1c(C2=CCC(F)(F)CC2)cnc2ccc(Cl)cc12]

Step 1. Synthesis of methyl 5-chloro-2-[[6-chloro-3-(4,4-difluorocyclohexen-1-yl)-4-quinolyl]amino]benzoate (2): To a stirred solution of methyl 2-[(3-bromo-6-chloro-4-quinolyl)amino]-5-chloro-benzoate (1 g, 2.35 mmol, 1 eq) and 2-(4,4-difluorocyclohexen-1-yl)-4,4,5,5-tetramethyl-1,3,2-dioxaborolane (572.85 mg, 2.35 mmol, 1 eq) in DMF (15 mL) and H₂O (3 mL) was added Pd(PPh₃)₄ (271.20 mg, 234.69 umol, 0.1 eq) and K₃PO₄ (1.49 g, 7.04 mmol, 3 eq), the mixture was purged with N₂ for 3 times, and stirred at 100° C. for 4 h. LCMS showed the starting material was consumed completely and desired product was detected. The reaction mixture was poured into water (50 mL). The aqueous phase was extracted with ethyl acetate (50 mL*2). The combined organic phase was dried with anhydrous Na₂SO₄, filtered and concentrated in vacuum. The crude product was purified by flash column (ISCO 10 g silica, 15-20% ethyl acetate in petroleum ether, gradient over 20 min). Based on TLC (Petroleum ether:Ethyl acetate=3/1, R_{f}=0.43). Compound methyl 5-chloro-2-[[6-chloro-3-(4,4-difluorocyclohexen-1-yl)-4-quinolyl]amino]benzoate (500 mg, 1.08 mmol, 45.98% yield) was obtained as a yellow solid. MS (M−H)⁻=463.1.

Step 2. Synthesis of 5-chloro-2-[[6-chloro-3-(4,4-difluorocyclohexen-1-yl)-4-quinolyl]amino]benzoic acid (344A): A solution of methyl 5-chloro-2-[[6-chloro-3-(4,4-difluorocyclohexen-1-yl)-4-quinolyl]amino]benzoate (80 mg, 172.67 umol, 1 eq) and LiOH·H₂O (2 M, 172.67 uL, 2 eq) in THF (1 mL) was stirred at 25° C. for 6 h. LCMS showed the starting material was consumed completely and desired product was detected. The reaction mixture was adjusted pH-5 by adding 2N HCl. The mixture was concentrate in vacuum. The residue was purified by prep-HPLC (column: Phenomenex Luna 80*30 mm*3 um; mobile phase: [water(0.4% HCl)-ACN]; B %: 20%-50%,8 min). Compound 5-chloro-2-[[6-chloro-3-(4,4-difluorocyclohexen-1-yl)-4-quinolyl]amino]benzoic acid (33.8 mg, 69.58 umol, 40.30% yield, 100% purity, HCl) was obtained as a yellow solid. ¹H NMR (400 MHz, DMSO-d6) δ ppm 10.23 (br s, 1H), 8.69 (s, 1H), 8.59 (br s, 1H), 8.15 (d, J=9.01 Hz, 1H), 8.00 (br d, J=8.88 Hz, 1H), 7.90 (d, J=2.50 Hz, 1H), 7.56 (br d, J=8.50 Hz, 1H), 6.94-7.08 (m, 1H), 5.66 (br s, 1H), 2.36-2.45 (m, 2H), 2.16-2.31 (m, 2H), 1.61 (br d, J=2.00 Hz, 2H). MS (M+H)⁺=449.0.

### Example 119—Synthesis of 345A

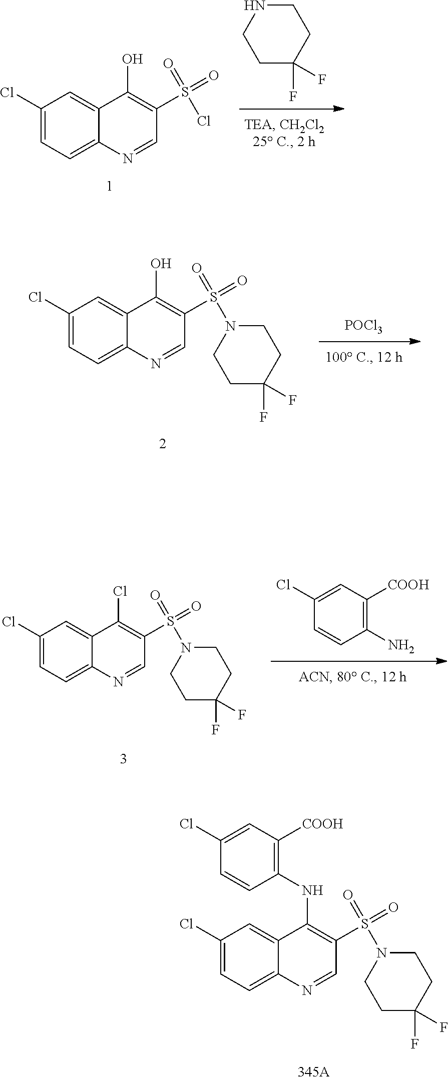

Step 1. Synthesis of 6-chloro-3-[(4,4-difluoro-1-piperidyl)sulfonyl]quinolin-4-ol (2): To a stirred solution of 6-chloro-4-hydroxy-quinoline-3-sulfonyl chloride (1 g, 3.60 mmol, 1 eq) in CH₂Cl₂ (20 mL) was added TEA (1.09 g, 10.79 mmol, 1.50 mL, 3 eq) and 4,4-difluoropiperidine (479.09 mg, 3.96 mmol, 1.1 eq), then the mixture was stirred at 25° C. for 1 h. LCMS showed the starting material was consumed completely and desired product was detected. The reaction mixture was concentrate in vacuum. Compound 6-chloro-3-[(4,4-difluoro-1-piperidyl)sulfonyl]quinolin-4-ol (1.3 g, 3.58 mmol, 99.66% yield) was obtained as a brown oil. MS (M+H)⁺=363.0.

Step 2. Synthesis of 4,6-dichloro-3-[(4,4-difluoro-1-piperidyl)sulfonyl]quinoline (3): A solution of 6-chloro-3-[(4,4-difluoro-1-piperidyl)sulfonyl]quinolin-4-ol (1 g, 2.76 mmol, 1 eq) in POCl₃ (8 mL) was stirred at 100° C. for 12 h under N₂. LCMS showed the starting material was consumed completely and desired product was detected. The reaction mixture was poured into ice water (40 mL) The aqueous phase was extracted with dichloromethane (40 mL*2). The combined organic phase was dried with anhydrous Na₂SO₄, filtered and concentrated in vacuum. The residue was purified by flash column (ISCO 10 g silica, 10-12% Ethyl acetate in Petroleum ether, gradient over 15 min). Based on TLC (Petroleum ether:Ethyl acetate=3/1, R_{f}=0.74). Compound 4,6-dichloro-3-[(4,4-difluoro-1-piperidyl)sulfonyl]quinoline (500 mg, 1.31 mmol, 47.58% yield) was obtained as a white solid. MS (M+H)⁺=381.1.

Step 3. Synthesis of 5-chloro-2-[[6-chloro-3-[(4,4-difluoro-1-piperidyl)sulfonyl]-4-quinolyl]amino]benzoic acid (345A): A solution of 4,6-dichloro-3-[(4,4-difluoro-1-piperidyl)sulfonyl]quinoline (100 mg, 262.31 umol, 1 eq) and 2-amino-5-chlorobenzoic acid (45.01 mg, 262.31 umol, 1 eq) in ACN (1 mL) was stirred at 80° C. for 12 h. LCMS showed the starting material was consumed completely and desired product was detected. The reaction mixture was concentrate in vacuum. The residue was purified by prep-HPLC (column: Phenomenex Luna 80*30 mm*3 um; mobile phase: [water(0.4% HCl)-ACN]; B %: 35%-70%,8 min). Compound 5-chloro-2-[[6-chloro-3-[(4,4-difluoro-1-piperidyl)sulfonyl]-4-quinolyl]amino]benzoic acid (24 mg, 43.01 umol, 16.40% yield, 99.06% purity, HCl) was obtained as a yellow solid. ¹H NMR (400 MHz, DMSO-d6) δ ppm 10.30-10.46 (m, 1H), 9.15 (s, 1H), 8.15 (d, J=9.00 Hz, 1H), 7.87-7.98 (m, 2H), 7.65 (d, J=2.25 Hz, 1H), 7.38 (dd, J=8.88, 2.50 Hz, 1H), 6.68 (d, J=9.01 Hz, 1H), 3.24 (br s, 4H), 1.89-2.04 (m, 2H), 1.72-1.89 (m, 2H). MS (M+H)⁺=516.0.

### Example 120—Synthesis of 346A

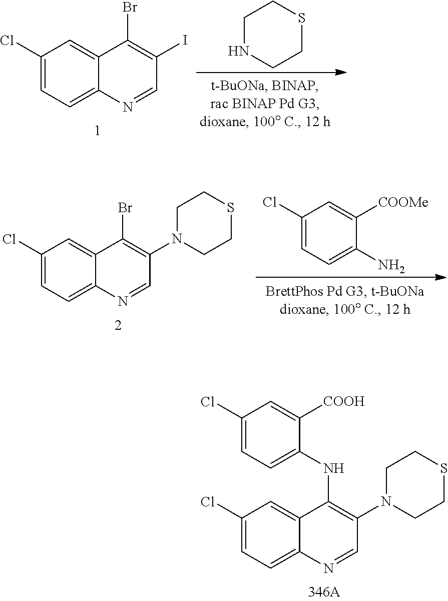

### Synthesis of 4-(4-bromo-6-chloro-3-quinolyl)thiomorpholine (2)

To a solution of 4-bromo-6-chloro-3-iodo-quinoline (300 mg, 814.34 umol, 1 eq) in dioxane (7 mL) was added BINAP (50.71 mg, 81.43 umol, 0.1 eq), [2-(2-aminophenyl)phenyl]-methylsulfonyloxy-palladium; [1-(2-diphenylphosphanyl-1-naphthyl)-2-naphthyl]-diphenyl-phosphane (80.82 mg, 81.43 μmol, 0.1 eq), t-BuONa (156.52 mg, 1.63 mmol, 2 eq) and thiomorpholine (100.83 mg, 977.21 μmol, 92.51 μL, 1.2 eq), the reaction was stirred at 100° C. for 12 h under N₂. LCMS showed starting material was consumed completely and the MS of desired product was detected. The reaction was cooled to ambient temperature, quenched with ice water (20 ml) and extracted with ethyl acetate (20 ml). The organic layer was washed with water, brine, dried over anhydrous sodium sulfate, filtered and concentrated in vacuum. The residue was purified by flash column (ISCO 20 g silica, 70-90% ethyl acetate in petroleum ether, gradient over 20 min). TLC (Petroleum ether/Ethyl acetate=2:1, R_{f}=0.35) Compound 4-(4-bromo-6-chloro-3-quinolyl)thiomorpholine (160 mg, 465.56 umol, 57.17% yield) was obtained as a yellow solid. MS (M+H)⁺=342.9.

### Synthesis of 5-chloro-2-[(6-chloro-3-thiomorpholino-4-quinolyl)amino]benzoic acid (346A)

To a solution of 4-(4-bromo-6-chloro-3-quinolyl)thiomorpholine (50 mg, 145.49 umol, 1 eq) in toluene (2 mL) was added t-BuONa (41.95 mg, 436.47 umol, 3 eq), RuPhos (6.79 mg, 14.55 umol, 0.1 eq), RuPhos Pd G3 (12.17 mg, 14.55 umol, 0.1 eq) and methyl 2-amino-5-chloro-benzoate (27.00 mg, 145.49 umol, 1 eq), the reaction was stirred at 100° C. for 12 h under Ar. LCMS showed starting material was consumed completely and the MS of desired product was detected. The reaction mixture was concentrated in vacuum. The crude product was purified by prep-HPLC (column: Phenomenex C18 75*30 mm*3 um; mobile phase: [water (10 mmol NH₄HCO₃)-ACN]; B %: 30%-50%, 8 min). Compound 5-chloro-2-[(6-chloro-3-thiomorpholino-4-quinolyl)amino]benzoic acid (3.8 mg, 8.06 umol, 5.54% yield, 95.08% purity) was obtained as a yellow solid. ¹H NMR (400 MHz, DMSO-d₆) δ=8.76 (s, 1H), 7.99 (d, J=8.9 Hz, 1H), 7.88 (d, J=2.5 Hz, 1H), 7.77 (d, J=1.9 Hz, 1H), 7.63 (dd, J=2.2, 8.9 Hz, 1H), 7.32 (dd, J=2.2, 8.9 Hz, 1H), 6.42 (d, J=9.0 Hz, 1H), 3.26 (br s, 4H), 2.45-2.42 (m, 4H). MS (M+H)⁺=434.1.

### Example 121—Synthesis of 349A

Synthetic scheme is shown in FIG. 39E.

### Synthesis of 6-chloro-4-hydroxy-quinoline-3-sulfonyl chloride (2)

5,6-chloroquinolin-4-ol (30 g, 167.04 mmol, 1 eq) was added to HSO₃Cl (210 mL) in portions and the mixture was stirred at 100° C. for 16 h. LCMS showed the starting material was consumed completely and desired MS was detected. The reaction mixture was poured into ice water and filtered. The filter cake was concentrated in vacuo. Compound 6-chloro-4-hydroxy-quinoline-3-sulfonyl chloride (40 g, 143.83 mmol, 86.11% yield) was obtained as a brown solid. ¹H NMR (400 MHz, DMSO-d6) δ=8.91 (s, 1H), 8.23 (d, J=2.3 Hz, 1H), 8.04-7.99 (m, 1H), 7.96-7.92 (m, 1H). MS (M+H)⁺=278.0.

### Synthesis of 6-chloro-3-thiomorpholinosulfonyl-quinolin-4-ol (3)

To a solution of 6-chloro-4-hydroxy-quinoline-3-sulfonyl chloride (15 g, 53.94 mmol, 1 eq) in DCM (150 mL) was added Et₃N (16.37 g, 161.81 mmol, 22.52 mL, 3 eq) and thiomorpholine (11.13 g, 107.87 mmol, 10.21 mL, 2 eq). The mixture was stirred at 25° C. for 2 h. LCMS showed the starting material was consumed completely and desired MS was detected. The reaction mixture was filtered and the filter cake was concentrated in vacuo. Compound 6-chloro-3-thiomorpholinosulfonyl-quinolin-4-ol (13.8 g, 40.02 mmol, 74.20% yield) was obtained as a yellow solid. ¹H NMR (400 MHz, DMSO-d6) δ=8.53 (s, 1H), 8.13-8.06 (m, 1H), 7.82-7.78 (m, 1H), 7.77-7.73 (m, 1H), 3.52-3.43 (m, 4H), 2.67-2.56 (m, 4H). MS (M+H)⁺=345.0.

### Synthesis of 4-[(4,6-dichloro-3-quinolyl)sulfonyl]thiomorpholine (4)

6-chloro-3-thiomorpholinosulfonyl-quinolin-4-ol (13.8 g, 40.02 mmol, 1 eq) was added to POCl₃ (240 mL) in portions and the mixture was stirred at 120° C. for 6 h. LCMS showed the starting material was consumed completely and desired MS was detected. The reaction mixture was concentrated to dryness to give the crude product. Then Ethyl acetate (30 mL) was added to it and poured into ice water (30 mL). The reaction mixture was extracted with Ethyl acetate (30 mL*3). The combined organic layers were washed with brine (20 mL), dried over Na₂SO₄ and concentrated to dryness to give residue. The crude product was purified by flash column (ISCO 20 g silica, 0-66% ethyl acetate in petroleum ether, gradient over 20 min). Compound 4-[(4,6-dichloro-3-quinolyl)sulfonyl]thiomorpholine (11.2 g, 30.83 mmol, 77.04% yield) was obtained as a yellow solid. MS (M+H)⁺=363.0.

### Synthesis of 5-chloro-2-[(6-chloro-3-thiomorpholinosulfonyl-4-quinolyl)amino]benzoic acid (349A)

To a solution of 4-[(4,6-dichloro-3-quinolyl)sulfonyl]thiomorpholine (2 g, 5.51 mmol, 1 eq) in EtOH (20 mL) and CHCl₃ (4 mL) was added 2-amino-5-chloro-benzoic acid (1.89 g, 11.01 mmol, 2 eq). The mixture was stirred at 80° C. for 2 h. LCMS showed 44% of starting material remained, 49% of desired product was detected. The reaction mixture was concentrated to dryness to give the crude product. The crude product was triturated with EtOH (30 mL) at 20° C. for 30 min. Then the solid was triturated with MeOH (20 mL) at 20° C. for 30 min. Compound 5-chloro-2-[(6-chloro-3-thiomorpholinosulfonyl-4-quinolyl)amino]benzoic acid (1 g, 1.91 mmol, 34.70% yield, 95.214% purity) was obtained as a yellow solid. ¹H NMR (400 MHz, METHANOL-d4) δ=9.25 (s, 1H), 8.18 (d, J=2.5 Hz, 1H), 8.13-8.08 (m, 1H), 8.06-8.01 (m, 1H), 7.65 (d, J=2.0 Hz, 1H), 7.55 (dd, J=2.6, 8.7 Hz, 1H), 7.23 (d, J=8.6 Hz, 1H), 3.60 (t, J=5.1 Hz, 4H), 2.72-2.60 (m, 4H). MS (M+H)⁺=498.0.

### Example 122—Synthesis of 353A

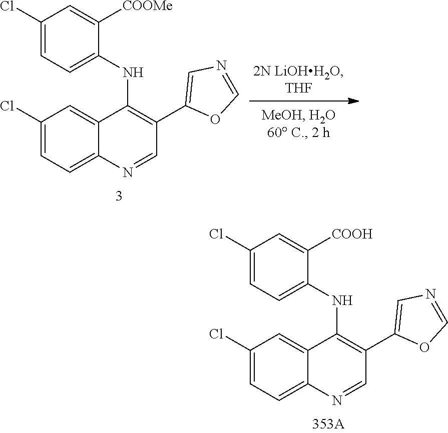 [SMILES: COC(=O)c1cc(Cl)ccc1Nc1c(-c2cnco2)cnc2ccc(Cl)cc12 ; O=C(O)c1cc(Cl)ccc1Nc1c(-c2cnco2)cnc2ccc(Cl)cc12]

### Synthesis of 5-chloro-2-[(6-chloro-3-oxazol-5-yl-4-quinolyl)amino]benzoic acid (353A)

To a stirred solution of methyl 5-chloro-2-[(6-chloro-3-oxazol-5-yl-4-quinolyl)amino]benzoate (20 mg, 48.28 umol, 1 eq) in THF (1 mL), MeOH (0.2 mL) and H₂O (0.2 mL) was added LiOH·H₂O (6.08 mg, 144.84 umol, 3 eq), the reaction was stirred at 60° C. for 2 h. LCMS showed starting material was consumed completely and the MS of desired product was detected. The reaction was adjusted pH˜4 by adding 2N HCl. Then the mixture was purified by prep-HPLC (column: Phenomenex Luna 80*30 mm*3 um; mobile phase: [water (0.04% HCl)-ACN]; B %: 35%-60%, 8 min). Compound 5-chloro-2-[(6-chloro-3-oxazol-5-yl-4-quinolyl)amino]benzoic acid (1.2 mg, 2.62 umol, 5.42% yield, 95.17% purity, HCl) was obtained as a yellow solid. ¹H NMR (400 MHz, DMSO-d₆) δ ppm 10.01-10.15 (m, 1H), 9.25 (s, 1H), 8.46 (s, 1H), 8.12-8.17 (m, 1H), 8.05-8.11 (m, 1H), 7.85-7.92 (m, 2H), 7.46-7.50 (m, 1H), 7.23 (br d, J=9.26 Hz, 1H), 6.29-6.36 (m, 1H). MS (M+H)⁺=399.9.

### Example 123—Synthesis of 354A

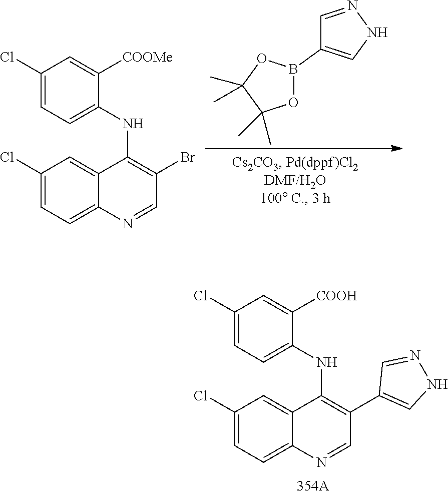 [SMILES: COC(=O)c1cc(Cl)ccc1Nc1c(Br)cnc2ccc(Cl)cc12 ; CC1(C)OB(c2cn[nH]c2)OC1(C)C ; O=C(O)c1cc(Cl)ccc1Nc1c(-c2cn[nH]c2)cnc2ccc(Cl)cc12]

### Synthesis of 5-chloro-2-[[6-chloro-3-(1H-pyrazol-4-yl)-4-quinolyl]amino]benzoic acid (354A)

To a stirred solution of methyl 2-[(3-bromo-6-chloro-4-quinolyl)amino]-5-chloro-benzoate (100 mg, 234.69 umol, 1 eq) in DMF (3 mL) and H₂O (0.5 mL) was added 4-(4,4,5,5-tetramethyl-1,3,2-dioxaborolan-2-yl)-1H-pyrazole (54.65 mg, 281.63 umol, 1.2 eq), Cs₂CO₃ (229.40 mg, 704.07 umol, 3 eq) and Pd(PPh₃)₄ (27.12 mg, 23.47 umol, 0.1 eq), the mixture was bubbled with N₂ for 1 minute, and stirred at 100° C. for 3 h. LCMS showed the starting material was consumed completely and desired product was detected. The reaction mixture was filtered, and filtrate was purified by prep-HPLC (column: Phenomenex Luna 80*30 mm*3 um; mobile phase: [water(0.04% HCl)-ACN]; B %: 30%-60%,8 min). Compound 5-chloro-2-[[6-chloro-3-(1H-pyrazol-4-yl)-4-quinolyl]amino]benzoic acid (13.1 mg, 29.23 umol, 12.46% yield, 97.23% purity, HCl) was obtained as a yellow solid. ¹H NMR (400 MHz, DMSO-d6) δ ppm 10.17 (br d, J=2.00 Hz, 1H), 9.13 (br d, J=2.13 Hz, 1H), 8.31-8.44 (m, 1H), 8.21 (br dd, J=8.82, 2.31 Hz, 1H), 7.95 (br d, J=8.88 Hz, 1H), 7.80 (br s, 3H), 7.25 (dd, J=8.88, 2.50 Hz, 1H), 6.53 (br s, 1H). MS (M+H)⁺=399.0.

### Example 124—Synthesis of 355A

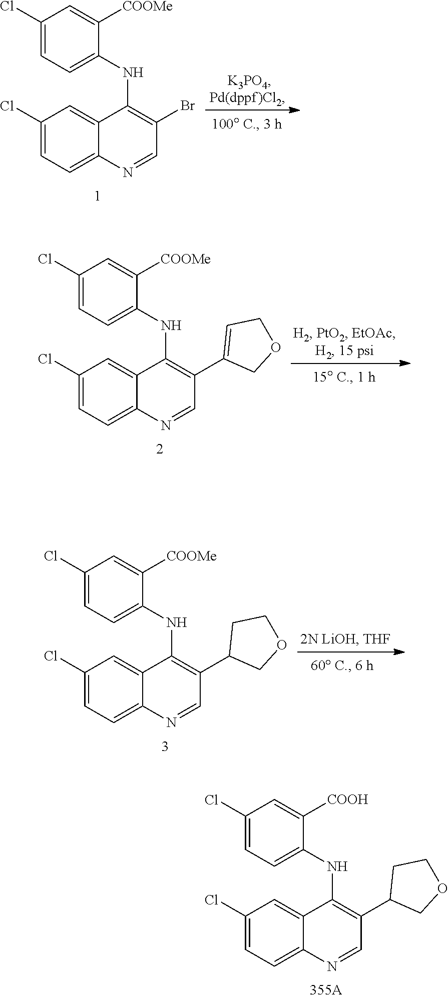 [SMILES: COC(=O)c1cc(Cl)ccc1Nc1c(Br)cnc2ccc(Cl)cc12 ; COC(=O)c1cc(Cl)ccc1Nc1c(C2=CCOC2)cnc2ccc(Cl)cc12 ; COC(=O)c1cc(Cl)ccc1Nc1c(C2CCOC2)cnc2ccc(Cl)cc12 ; O=C(O)c1cc(Cl)ccc1Nc1c(C2CCOC2)cnc2ccc(Cl)cc12]

### Synthesis of methyl 5-chloro-2-[[6-chloro-3-(2,5-dihydrofuran-3-yl)-4-quinolyl]amino]benzoate (2)

To a solution of methyl 2-[(3-bromo-6-chloro-4-quinolyl)amino]-5-chloro-benzoate (326.01 mg, 765.11 umol, 1 eq) in DMF (0.5 mL) and H₂O (0.1 mL) was added Pd(dppf)Cl₂ (55.98 mg, 76.51 umol, 0.1 eq), K₃PO₄ (487.23 mg, 2.30 mmol, 3 eq) and 2-(2,5-dihydrofuran-3-yl)-4,4,5,5-tetramethyl-1,3,2-dioxaborolane (150 mg, 765.11 umol, 1 eq), the mixture was bubbled with N₂, the reaction was stirred at 100° C. for 3 h. under N₂. LCMS showed starting material was consumed completely and the MS of desired product was detected. The reaction was cooled to ambient temperature, quenched with water (20 ml) and extracted with ethyl acetate (20 ml). The organic layer was washed with water, brine, dried over anhydrous sodium sulfate, filtered and concentrated in vacuum. The residue was purified by flash column (ISCO 20 g silica, 10-60% ethyl acetate in petroleum ether, gradient over 20 mm). Based on TLC (Petroleum ether/Ethyl acetate=3:1, R_{f}=0.53) Compound methyl 5-chloro-2-[[6-chloro-3-(2,5-dihydrofuran-3-yl)-4-quinolyl]amino]benzoate (150 mg, 361.21 umol, 47.21% yield) was obtained as a yellow oil. MS (M+H)⁺=415.1.

### Synthesis of methyl 5-chloro-2-[(6-chloro-3-tetrahydrofuran-3-yl-4-quinolyl)amino]benzoate (3)

To a solution of methyl 5-chloro-2-[[6-chloro-3-(2,5-dihydrofuran-3-yl)-4-quinolyl]amino]benzoate (65 mg, 156.53 umol, 1 eq) in THF (0.5 mL) was added PtO₂ (3.55 mg, 15.65 umol, 0.1 eq), the mixture was purged with H₂, the reaction was stirred at 15° C. for 1 h under H₂. LCMS showed starting material was consumed completely and the MS of desired product was detected. The reaction was cooled to ambient temperature, quenched with water (10 ml) and extracted with ethyl acetate (10 ml). The organic layer was washed with water, brine, dried over anhydrous sodium sulfate, filtered and concentrated in vacuum. Compound methyl 5-chloro-2-[(6-chloro-3-tetrahydrofuran-3-yl-4-quinolyl)amino]benzoate (65 mg, 155.77 umol, 99.52% yield) was obtained as a yellow oil. MS (M+H)⁺=417.1.

### Synthesis of 5-chloro-2-[(6-chloro-3-tetrahydrofuran-3-yl-4-quinolyl)amino]benzoic acid (355A)

To a solution of methyl 5-chloro-2-[(6-chloro-3-tetrahydrofuran-3-yl-4-quinolyl)amino]benzoate (65 mg, 155.77 umol, 1 eq) in THF (2 mL), MeOH (0.1 mL) and H₂O (0.1 mL) was added LiOH·H₂O (13.07 mg, 311.54 umol, 2 eq), the reaction was stirred at 60° C. for 4 h. LCMS showed starting material was consumed completely and the MS of desired product was detected. The reaction mixture was concentrated in vacuum. The crude product was purified by prep-HPLC (column: Phenomenex luna C18 80*40 mm*3 um; mobile phase:[water(0.04% HCl)-ACN]; B %: 25%-55%,7 min) Compound 5-chloro-2-[(6-chloro-3-tetrahydrofuran-3-yl-4-quinolyl)amino]benzoic acid (3.70 mg, 8.13 umol, 5.22% yield, 96.63% purity, HCl) was obtained as a yellow solid. ¹H NMR (400 MHz, DMSO-d₆+D2O, T=273+80K) δ=8.90 (s, 1H), 8.20-8.05 (m, 1H), 7.93 (d, J=2.6 Hz, 1H), 7.87-7.75 (m, 2H), 7.38 (dd, J=2.6, 8.9 Hz, 1H), 6.52 (d, J=8.8 Hz, 1H), 4.00 (dt, J=5.1, 8.3 Hz, 1H), 3.95-3.88 (m, 1H), 3.83-3.70 (m, 2H), 3.69-3.58 (m, 1H), 2.38-2.21 (m, 1H), 2.09-1.96 (m, 1H). MS (M+H)⁺=402.9.

### Example 125—Synthesis of 357A

Synthetic scheme is shown in FIG. 39E.

### Synthesis of tert-butyl 3-[6-chloro-4-(4-chloro-2-methoxycarbonyl-anilino)-3-quinolyl]-2,5-dihydropyrrole-1-carboxylate (2)

To a solution of methyl 2-[(3-bromo-6-chloro-4-quinolyl)amino]-5-chloro-benzoate (1 g, 2.35 mmol, 1 eq) in DMF (6 mL) and H₂O (1 mL) was added K₃PO₄ (1.49 g, 7.04 mmol, 3 eq), Pd(dppf)Cl₂ (171.73 mg, 234.69 umol, 0.1 eq) and tert-butyl 3-(4,4,5,5-tetramethyl-1,3,2-dioxaborolan-2-yl)-2,5-dihydropyrrole-1-carboxylate (692.77 mg, 2.35 mmol, 1 eq) the mixture was purged with N₂, the reaction was stirred at 100° C. for 3 h under N₂. LCMS showed starting material was consumed completely and the MS of desired product was detected. The reaction was cooled to ambient temperature, quenched with water (20 ml) and extracted with ethyl acetate (20 ml). The organic layer was washed with water, brine, dried over anhydrous sodium sulfate, filtered and concentrated in vacuum. The residue was purified by flash column (ISCO 20 g silica, 40-70% ethyl acetate in petroleum ether, gradient over 20 min). Based on TLC (Petroleum ether:Ethyl acetate=0/1, R_{f}=0.48). Compound tert-butyl 3-[6-chloro-4-(4-chloro-2-methoxycarbonyl-anilino)-3-quinolyl]-2,5-dihydropyrrole-1-carboxylate (600 mg, 1.17 mmol, 49.70% yield) was obtained as a yellow solid. MS (M+H)⁺=514.2.

### Synthesis of tert-butyl 3-(6-chloro-4-((4-chloro-2-(methoxycarbonyl)phenyl)amino)quinolin-3-yl)pyrrolidine-1-carboxylate (3)

To a solution of tert-butyl 3-[6-chloro-4-(4-chloro-2-methoxycarbonyl-anilino)-3-quinolyl]-2,5-dihydropyrrole-1-carboxylate (200 mg, 388.80 umol, 1 eq) in EtOAc (3 mL) was added PtO2 (8.83 mg, 38.88 umol, 0.1 eq), the mixture was purged with H₂, the reaction was stirred at 15° C. for 3 h. LCMS showed starting material was consumed completely and the MS of desired product was detected. The reaction mixture was concentrated under reduced pressure to give a residue. Compound tert-butyl 3-[6-chloro-4-(4-chloro-2-methoxycarbonyl-anilino)-3-quinolyl]pyrrolidine-1-carboxylate (150 mg, 290.46 umol, 74.71% yield) was obtained as a yellow solid. MS (M+H)⁺=516.2.

### Synthesis of 2-((3-(1-(tert-butoxycarbonyl) pyrrolidin-3-yl)-6-chloroquinolin-4-yl)amino)-5-chlorobenzoic acid (4)

To a solution of tert-butyl 3-[6-chloro-4-(4-chloro-2-methoxycarbonyl-anilino)-3-quinolyl]pyrrolidine-1-carboxylate (150 mg,290.46 umol, 1 eq) in THF (2 mL), MeOH (0.4 mL) and H₂O (0.4 mL) was added LiOH·H₂O (24.38 mg, 580.93 umol, 2 eq), the reaction was stirred at 60° C. for 2 h. LCMS showed starting material was consumed completely and the MS of desired product was detected. The reaction mixture was concentrated under reduced pressure to give a residue. Compound2-[[3-(1-tert-butoxycarbonylpyrrolidin-3-yl)-6-chloro-4-quinolyl]amino]-5-chloro-benzoic acid (140 mg, 278.67 umol, 95.94% yield) was obtained as a yellow solid. MS (M+H)⁺=502.1.

### Synthesis of 5-chloro-2-((6-chloro-3-(pyrrolidin-3-yl)quinolin-4-yl)amino)benzoic acid (357A)

A solution of 2-[[3-(1-tert-butoxycarbonylpyrrolidin-3-yl)-6-chloro-4-quinolyl]amino]-5-chloro-benzoic acid (140 mg, 278.67 umol, 1 eq) in HCl/EtOAc (2 mL), the reaction was stirred at 15° C. for 1 h. LCMS showed starting material was consumed completely and the MS of desired product was detected. The reaction mixture was concentrated under reduced pressure to give a residue. The residue was purified by prep-HPLC (column: Phenomenex Luna 80*30 mm*3 um; mobile phase: [water(0.04% HCl)-ACN]; B %: 30%-45%,8 min). Afford 10.3 mg crude product. The crude product was purified by prep-HPLC (column: Phenomenex C18 75*30 mm*3 um; mobile phase: [water(0.04% HCl)-ACN]; B %: 10%-55%,8 min). Compound 5-chloro-2-[(6-chloro-3-pyrrolidin-3-yl-4-quinolyl)amino]benzoic acid (9.9 mg, 22.56 umol, 8.10% yield, 100% purity, HCl) was obtained as a yellow solid. ¹H NMR (400 MHz, DMSO-d₆) δ=9.06 (s, 1H), 8.12 (d, J=9.0 Hz, 1H), 7.90 (d, J=2.6 Hz, 1H), 7.82 (dd, J=2.1, 9.0 Hz, 1H), 7.78 (br d, J=5.6 Hz, 1H), 7.31 (dd, J=2.6, 8.9 Hz, 1H), 6.25 (t, J=9.1 Hz, 1H), 3.72-3.64 (m, 1H), 3.62-3.37 (m, 2H), 3.36-3.13 (m, 2H), 2.38-2.00 (m, 2H). MS (M+H)⁺=402.0.

### Example 126—Synthesis of 359A

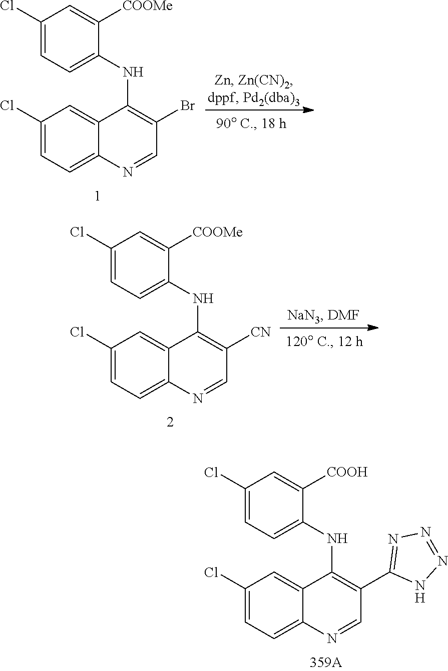 [SMILES: COC(=O)c1cc(Cl)ccc1Nc1c(Br)cnc2ccc(Cl)cc12 ; COC(=O)c1cc(Cl)ccc1Nc1c(C#N)cnc2ccc(Cl)cc12 ; O=C(O)c1cc(Cl)ccc1Nc1c(-c2nnn[nH]2)cnc2ccc(Cl)cc12]

### Synthesis of methyl 5-chloro-2-[(6-chloro-3-cyano-4-quinolyl)amino]benzoate (2)

To a solution of methyl 2-[(3-bromo-6-chloro-4-quinolyl)amino]-5-chloro-benzoate (1 g, 2.35 mmol, 1 eq) in DMF (20 mL) was added Zn(CN)₂ (0.470 g, 4.00 mmol, 254.05 uL, 1.71 eq), Zn (0.060 g, 917.57 umol, 3.91e-1 eq), DPPF (130.11 mg, 234.69 umol, 0.1 eq) and Pd2(dba)₃ (107.46 mg, 117.35 umol, 0.05 eq), the mixture was purged with N₂, the reaction was stirred at 90° C. for 18 h under N₂. LCMS showed starting material was consumed completely and the MS of desired product was detected. The reaction mixture was quenched with water and the aqueous layer was extracted with ethyl acetate (3*50 mL). The combined organic layer was and washed with aqueous ammonia (2*50 mL), water, dried over Na₂SO₄, filtered and concentrated to afford crude product The residue was purified by flash column (ISCO 40 g silica, 50-60% ethyl acetate in petroleum ether, gradient over 20 min). TLC (Petroleum ether/Ethyl acetate=1:1, R_{f}=0.39). Compound methyl 5-chloro-2-[(6-chloro-3-cyano-4-quinolyl)amino]benzoate (300 mg, 806.01 umol, 34.34% yield) was obtained as a yellow solid. MS (M+H)⁺=372.1.

### Synthesis of 5-chloro-2-[[6-chloro-3-(1H-tetrazol-5-yl)-4-quinolyl]amino]benzoic acid (359A)

To a solution of methyl 5-chloro-2-[(6-chloro-3-cyano-4-quinolyl)amino]benzoate (80 mg, 214.94 umol, 1 eq) in DMF (1 mL) was added NaN₃ (24.00 mg, 369.17 umol, 1.72 eq) the mixture was purged with N₂, the reaction was stirred at 120° C. for 12 h. LCMS showed starting material was consumed completely and the MS of desired product was detected. The reaction was cooled to ambient temperature, quenched with water (5 ml). The mixture was adjusted pH=9 by adding 2M KOH (5 ml) solution slowly at 0° C. and extracted with ethyl acetate (10 ml). The organic layer was washed with water, brine, dried over anhydrous sodium sulfate, filtered and concentrated in vacuum. The crude product was purified by prep-HPLC (column: Phenomenex C18 75*30 mm*3 um; mobile phase: [water(10 mmol NH₄HCO₃)-ACN]; B %: 15%-35%,8 min). Compound 5-chloro-2-[[6-chloro-3-(1H-tetrazol-5-yl)-4-quinolyl]amino]benzoic acid (5 mg, 12.46 umol, 5.80% yield, 100% purity) was obtained as a yellow solid. ¹H NMR (400 MHz, DMSO-d₆) δ=9.29 (s, 1H), 8.07 (d, J=8.9 Hz, 1H), 7.83-7.78 (m, 2H), 7.78-7.75 (m, 1H), 7.18 (dd, J=2.6, 8.9 Hz, 1H), 6.40 (d, J=9.0 Hz, 1H). MS (M+H)⁺=400.9/

### Example 127—Synthesis of 361A

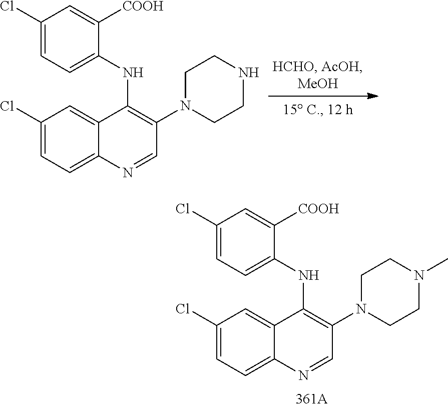 [SMILES: O=C(O)c1cc(Cl)ccc1Nc1c(N2CCNCC2)cnc2ccc(Cl)cc12 ; CN1CCN(c2cnc3ccc(Cl)cc3c2Nc2ccc(Cl)cc2C(=O)O)CC1]

### Synthesis of 5-chloro-2-[[6-chloro-3-(4-methylpiperazin-1-yl)-4-quinolyl]amino]benzoic acid (361A)

To a solution of 5-chloro-2-[(6-chloro-3-piperazin-1-yl-4-quinolyl)amino]benzoic acid (6 mg, 14.38 umol, 1 eq) in MeOH (0.5 mL) was added AcOH (172.69 ug, 2.88 umol, 1.64e-1 uL, 0.2 eq) and HCHO (1.30 mg, 43.14 umol, 1.19 uL, 3.00 eq), NaBH₃CN (1.8 mg, 28.76 umol, 2 eq), the reaction was stirred at 15° C. for 12 h. LCMS showed starting material was consumed completely and the MS of desired product was detected. The reaction mixture was concentrated in vacuum. The crude product was purified by prep-HPLC (column: Waters Xbridge BEH C18 100*30 mm*10 um; mobile phase: [water(10 mmol NH₄HCO₃)-ACN]; B %: 15%-45%,81 min) Compound 5-chloro-2-[[6-chloro-3-(4-methylpiperazin-1-yl)-4-quinolyl]amino]benzoic acid (1.3 mg, 2.98 umol, 20.70% yield, 98.76% purity) was obtained as a yellow solid. ¹H NMR (400 MHz, DMSO-d₆) δ=8.74 (s, 1H), 7.96 (d, J=8.9 Hz, 1H), 7.85 (d, J=2.7 Hz, 1H), 7.79 (d, J=2.1 Hz, 1H), 7.59 (dd, J=2.3, 8.9 Hz, 1H), 7.22 (dd, J=2.6, 8.8 Hz, 1H), 6.35 (d, J=8.8 Hz, 1H), 3.09 (br s, 4H), 2.45-2.32 (m, 4H), 2.28-2.21 (m, 3H). MS (M+H)⁺=431.1.

### Example 128—Synthesis of 362A

Synthetic scheme is shown in FIG. 39G.

### Synthesis of tert-butyl 4-(4-bromo-6-chloro-3-quinolyl)piperazine-1-carboxylate (2)

To a solution of 4-bromo-6-chloro-3-iodo-quinoline (300 mg, 814.34 umol, 1 eq) in toluene (4 mL) was added t-BuONa (234.78 mg, 2.44 mmol, 3 eq), BINAP (50.71 mg, 81.43 umol, 0.1 eq), [2-(2-aminophenyl)phenyl]-methylsulfonyloxypalladium; [1-(2-diphenylphosphanyl-1-naphthyl)-2-naphthyl]-diphenyl-phosphane (80.82 mg, 81.43 umol, 0.1 eq) and tertbutylpiperazine-1-carboxylate; hydrochloride (199.50 mg, 895.78 umol, 1.1 eq), the reaction was stirred at 100° C. for 12 h under Ar. LCMS showed starting material was consumed completely and the MS of desired product was detected. The reaction was cooled to ambient temperature, quenched with water (20 ml) and extracted with ethyl acetate (20 ml). The organic layer was washed with water, brine, dried over anhydrous sodium sulfate, filtered and concentrated in vacuum. The residue was purified by flash column (ISCO 20 g silica, 10-40% ethyl acetate in petroleum ether, gradient over 20 min). TLC (Petroleum ether/Ethyl acetate=2:1, R_{f}=0.40) Compound tert-butyl 4-(4-bromo-6-chloro-3-quinolyl)piperazine-1-carboxylate (160 mg, 374.94 umol, 46.04% yield) was obtained as a white solid. MS (M+H)⁺=428.0.

### Synthesis of 2-[[3-(4-tert-butoxycarbonylpiperazin-1-yl)-6-chloro-4-quinolyl]amino]-5-chloro-benzoic acid (3)

To a solution of tert-butyl 4-(4-bromo-6-chloro-3-quinolyl)piperazine-1-carboxylate (60 mg, 140.60 umol, 1 eq) in toluene (3 mL) was added t-BuONa (40.54 mg, 421.80 umol, 3 eq), RuPhos (6.56 mg, 14.06 umol, 0.1 eq), RuPhos Pd G3 (11.76 mg, 14.06 umol, 0.1 eq) and methyl 2-amino-5-chloro-benzoate (28.71 mg, 154.66 umol, 1.1 eq), the reaction was purged with Ar, the reaction was stirred at 100° C. for 12 h under Ar. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04HCl)-ACN]; B %: 35%-65%,8 min). Compound 2-[[3-(4-tert-butoxycarbonylpiperazin-1-yl)-6-chloro-4-quinolyl]amino]-5-chloro-benzoic acid (20 mg, 38.65 umol,27.49% yield) was obtained as a yellow solid. MS (M+H)⁺=517.1.

### Synthesis of 5-chloro-2-[(6-chloro-3-piperazin-1-yl-4-quinolyl)amino]benzoic acid (362A)

A solution of 2-[[3-(4-tert-butoxycarbonylpiperazin-1-yl)-6-chloro-4-quinolyl]amino]-5-chloro-benzoic acid (20 mg, 38.65 umol, 1 eq) in HCl/EtOAc (4 mL), the reaction was stirred at 15° C. for 0.5 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 15%-40%,8 min). Compound 5-chloro-2-[(6-chloro-3-piperazin-1-yl-4-quinolyl)amino]benzoic acid (10.6 mg, 25.05 umol, 64.81% yield, 98.62% purity) was obtained as a yellow solid. ¹H NMR (400 MHz, DMSO-d₆) δ=10.35-10.06 (m, 1H), 9.21-8.87 (m, 2H), 8.85 (s, 1H), 8.39-8.24 (m, 1H), 8.18 (br d, J=9.0 Hz, 1H), 7.89 (d, J=2.6 Hz, 2H), 7.57 (br d, J=8.2 Hz, 1H), 7.22-6.90 (m, 1H), 3.14 (br s, 4H), 2.80-2.68 (m, 4H). MS (M+H)⁺=417.0.

### Example 129—Synthesis of 367A

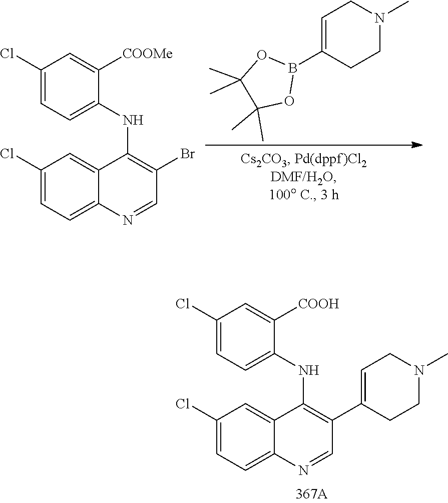

### Synthesis of 5-chloro-2-[[6-chloro-3-(1-methyl-3,6-dihydro-2H-pyridin-4-yl)-4-quinolyl]amino]benzoic acid (367A)

To a stirred solution of methyl 2-[(3-bromo-6-chloro-4-quinolyl)amino]-5-chloro-benzoate (100 mg, 234.69 umol, 1 eq) in DMF (4 mL) and H₂O (1 mL) was added 1-methyl-4-(4,4,5,5-tetramethyl-1,3,2-dioxaborolan-2-yl)-3,6-dihydro-2H-pyridine (52.36 mg, 234.69 umol, 1 eq), Pd(dppf)Cl₂ (17.17 mg, 23.47 umol, 0.1 eq) and Cs₂CO₃ (229.40 mg, 704.07 umol, 3 eq), the mixture was purged with N₂ for 1 minute, and stirred at 100° C. for 3 h. LCMS showed the starting material was consumed completely and desired product was detected. The reaction mixture was filtered, and filtrate was purified by prep-HPLC (column: Phenomenex Luna C18 150*30 nm*5 um; mobile phase: [water(0.1% TFA)-ACN]; B %: 25%-60%,8 min). Compound 5-chloro-2-[[6-chloro-3-(1-methyl-3,6-dihydro-2H-pyridin-4-yl)-4-quinolyl]amino]benzoic acid (7.4 mg, 13.34 umol, 5.68% yield, 97.74% purity, TFA) was obtained as a yellow solid. ¹H NMR (400 MHz, DMSO-d6+D₂O) δ ppm 8.63 (s, 1H), 8.00 (d, J=8.88 Hz, 1H), 7.81 (t, J=2.25 Hz, 2H), 7.74 (dd, J=8.94, 2.31 Hz, 1H), 7.08 (dd, J=8.76, 2.75 Hz, 1H), 6.29 (d, J=8.76 Hz, 1H), 5.86 (br s, 1H), 3.64 (br s, 2H), 2.93-3.10 (m, 2H), 2.67 (s, 3H), 2.42 (br s, 2H). MS (M+H)⁺=428.0.

### Example 130—Synthesis of 368A

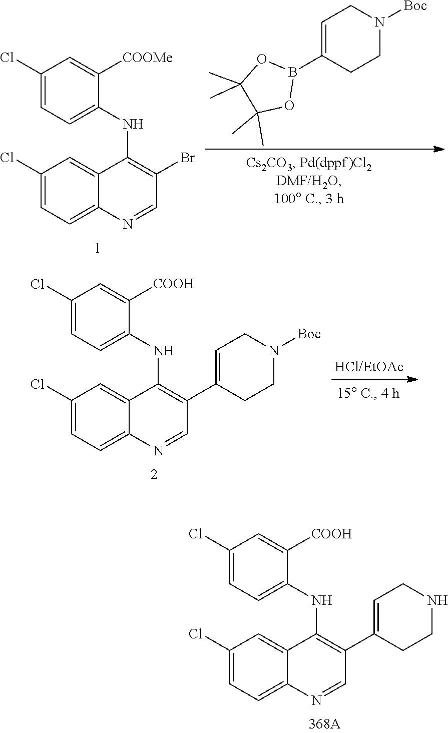 [SMILES: COC(=O)c1cc(Cl)ccc1Nc1c(Br)cnc2ccc(Cl)cc12 ; CC(C)(C)OC(=O)N1CC=C(B2OC(C)(C)C(C)(C)O2)CC1 ; CC(C)(C)OC(=O)N1CC=C(c2cnc3ccc(Cl)cc3c2Nc2ccc(Cl)cc2C(=O)O)CC1 ; O=C(O)c1cc(Cl)ccc1Nc1c(C2=CCNCC2)cnc2ccc(Cl)cc12]

### Synthesis of 2-[[3-(1-tert-butoxycarbonyl-3,6-dihydro-2H-pyridin-4-yl)-6-chloro-4-quinolyl]amino]-5-chloro-benzoic acid (2)

To a stirred solution of methyl 2-[(3-bromo-6-chloro-4-quinolyl)amino]-5-chloro-benzoate (100 mg, 234.69 umol, 1 eq) in DMF (4 mL) and H₂O (1 mL) was added tert-butyl 4-(4,4,5,5-tetramethyl-1,3,2-dioxaborolan-2-yl)-3,6-dihydro-2H-pyridine-1-carboxylate (72.57 mg, 234.69 umol, 1 eq), Cs₂CO₃ (229.40 mg, 704.07 umol, 3 eq) and Pd(dppf)Cl₂ (17.17 mg, 23.47 umol, 0.1 eq), the mixture was bubbled with N₂ for 1 minute, and stirred at 100° C. for 3 h. LCMS showed the starting material was consumed completely and desired product was detected. The reaction mixture was filtered, and filtrate was purified by prep-HPLC (column: Waters Xbridge BEH C18 100*30 mm*10 um; mobile phase: [water(NH₄HCO₃)-ACN]; B %: 30%-60%,8 min). Compound 2-[[3-(1-tert-butoxycarbonyl-3,6-dihydro-2H-pyridin-4-yl)-6-chloro-4-quinolyl]amino]-5-chloro-benzoic acid (30 mg, 58.32 umol, 24.85% yield) was obtained as a yellow solid. MS (M+H)⁺=514.1.

### Synthesis of 5-chloro-2-[[6-chloro-3-(1,2,3,6-tetrahydropyridin-4-yl)-4-quinolyl]amino]benzoic acid (368A)

A solution of 2-[[3-(1-tert-butoxycarbonyl-3,6-dihydro-2H-pyridin-4-yl)-6-chloro-4-quinolyl]amino]-5-chloro-benzoic acid (30 mg, 58.32 umol, 1 eq) in HCl/EtOAc (4 M, 2 mL, 137.17 eq) was stirred at 15° C. for 4 h. LCMS showed the starting material was consumed completely and desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 10%-40%,8 min). Compound 5-chloro-2-[[6-chloro-3-(1,2,3,6-tetrahydropyridin-4-yl)-4-quinolyl]amino]benzoic acid (20.80 mg, 45.95 umol, 78.79% yield, 99.58% purity, HCl) was obtained as a yellow solid. ¹H NMR (400 MHz, DMSO-d6) δ ppm 10.35 (br s, 1H), 9.30 (br s, 2H), 8.57 (br d, J=2.75 Hz, 2H), 8.15-8.31 (m, 1H), 8.03 (br d, J=9.01 Hz, 1H), 7.90 (d, J=2.50 Hz, 1H), 7.60 (br d, J=8.63 Hz, 1H), 7.12 (br s, 1H), 5.84 (br s, 1H), 3.42 (br s, 2H), 2.53-2.65 (m, 2H), 2.27-2.44 (m, 2H). MS (M+H)⁺=414.0

### Example 131—Synthesis of 371A

Synthetic scheme is shown in FIG. 39H.

### Synthesis of 4,6-dichloroquinoline-3-carbaldehyde (1)

POCl₃ (37.97 g, 247.63 mmol, 23.01 mL, 6 eq) was added dropwise to DMF (50 mL) at 0° C. and stirred at 20° C. for 15 min. Then 1-(2-amino-5-chloro-phenyl)ethanone (7 g, 41.27 mmol, 1 eq) in DMF (10 mL) was added dropwise to the above mixture at 0° C. and stirred at 90° C. for 12 h under N₂ atmosphere. LCMS showed the starting material was consumed completely and desired MS was detected. The reaction mixture was concentrated to dryness to give the crude product. Then the residue was quenched by ice water (70 mL) and basified by sat. NaHCO₃ to pH=8-10 at 0° C. The reaction mixture was extracted with Ethyl acetate (70 mL*3). The combined organic layers were washed with brine (50 mL) dried over Na₂SO₄ and concentrated to dryness to give residue. The crude product was purified by flash column (ISCO 20 g silica, 0-6% ethyl acetate in petroleum ether, gradient over 20 min). Compound 4,6-dichloroquinoline-3-carbaldehyde (5 g, crude) was obtained as a pale yellow solid. MS (M+H)⁺=226.0.

### Synthesis of 4-[(4,6-dichloro-3-quinolyl)methyl]morpholine (2)

To a solution of 4,6-dichloroquinoline-3-carbaldehyde (4.5 g, 19.91 mmol, 1 eq) in MeOH (60 mL) was added morpholine (3.47 g, 39.81 mmol, 3.50 mL, 2 eq) and AcOH until PH=4-6 at 0° C. Then the mixture was stirred at 20° C. for 2 h. Then NaBH₃CN (2.50 g, 39.81 mmol, 2 eq) was added to the above mixture at 0° C. in portions and stirred at 20° C. for 12 h. LCMS showed the starting material was consumed completely and desired MS was detected. The reaction mixture was concentrated to dryness to give the crude product. 50 mL of water was added to the reaction. Then the mixture was cooled to 0° C. and sat. NaHCO₃ was added to it until PH=8˜10. The reaction mixture was extracted with DCM (50 mL*3). The combined organic layers were washed with brine (40 mL), dried over Na₂SO₄ and concentrated to dryness to give residue. The crude product was purified by flash column (ISCO 40 g silica, 0-47% ethyl acetate in petroleum ether, gradient over 20 min). Compound 4-[(4,6-dichloro-3-quinolyl)methyl]morpholine (3.1 g, 10.43 mmol, 52.40% yield) was obtained as a white solid. ¹H NMR (400 MHz, CHLOROFORM-d) δ 8.97 (s, 1H), 8.25 (d, J=2.3 Hz, 1H), 8.05 (d, J=9.0 Hz, 1H), 7.69 (dd, J=2.3, 8.9 Hz, 1H), 3.86 (s, 2H), 3.78-3.70 (m, 4H), 2.63-2.51 (m, 4H). MS (M+H)⁺=297.1.

### Synthesis of methyl 5-chloro-2-[[6-chloro-3-(morpholinomethyl)-4-quinolyl]amino]benzoate (3)

A mixture of 4-[(4,6-dichloro-3-quinolyl)methyl]morpholine (3 g, 10.09 mmol, 1 eq), methyl 2-amino-5-chloro-benzoate (1.87 g, 10.09 mmol, 1 eq), XPhos Pd G3 (854.48 mg, 1.01 mmol, 0.1 eq), Cs₂CO₃ (6.58 g, 20.19 mmol, 2 eq) in dioxane (50 mL) was degassed and purged with N₂ for 3 times, and then the mixture was stirred at 110° C. for 16 h under N₂ atmosphere. LCMS showed 15% of starting material remained, 50% of desired product was detected. The reaction mixture was filtered. 40 mL of water was added to the filtrate and extracted with Ethyl acetate (40 mL*3). The combined organic layers were washed with brine (30 mL) dried over Na₂SO₄ and concentrated to dryness to give residue. The crude product was purified by flash column (ISCO 20 g silica, 0-100% ethyl acetate in petroleum ether, gradient over 20 min). Compound methyl 5-chloro-2-[[6-chloro-3-(morpholinomethyl)-4-quinolyl]amino]benzoate (710 mg, 1.59 mmol, 15.76% yield) was obtained as a white solid. ¹H NMR (400 MHz, CHLOROFORM-d) δ 10.29 (s, 1H), 8.79 (s, 1H), 8.08-8.00 (m, 2H), 7.67 (d, J=2.3 Hz, 1H), 7.61 (dd, J=2.3, 8.9 Hz, 1H), 7.14 (dd, J=2.6, 8.9 Hz, 1H), 6.29 (d, J=8.9 Hz, 1H), 3.98 (s, 3H), 3.89-3.68 (m, 6H), 2.46 (br d, J=4.1 Hz, 4H). MS (M+H)⁺=446.10.

### Synthesis of 5-chloro-2-[[6-chloro-3-(morpholinomethyl)-4-quinolyl]amino]benzoic acid (371A)

To a solution of methyl 5-chloro-2-[[6-chloro-3-(morpholinomethyl)-4-quinolyl]amino]benzoate (700 mg, 1.57 mmol, 1 eq) in THF (7 mL), MeOH (2.1 mL) and H₂O (0.7 mL) was added LiOH·H₂O (131.62 mg, 3.14 mmol, 2 eq). The mixture was stirred at 60° C. for 2 h. LCMS showed the starting material was consumed completely and desired MS was detected. The reaction mixture was concentrated to dryness to give the crude product. 6 mL of water was added to the reaction mixture, then 2M HCl was added to the above mixture at 0° C. until PH=5-6. Then the mixture was extracted with Ethyl acetate (15 mL*3). The combined organic layers were washed with brine (4 mL), dried over Na₂SO₄ and concentrated to dryness to give residue. The crude product was purified by flash column (ISCO 20 g silica, 0-100% ethyl acetate in petroleum ether; 0-13% methanol in dichloromethane, gradient over 20 min). Compound 5-chloro-2-[[6-chloro-3-(morpholinomethyl)-4-quinolyl]amino]benzoic acid (203.70 mg, 460.42 μmol, 29.36% yield, 97.712% purity) was obtained as a yellow solid. ¹H NMR (400 MHz, DMSO-d6) δ 8.84 (s, 1H), 8.07 (d, J=9.0 Hz, 1H), 7.89 (d, J=2.6 Hz, 1H), 7.74 (dd, J=2.4, 9.0 Hz, 1H), 7.60 (d, J=2.3 Hz, 1H), 7.28 (dd, J=2.7, 8.9 Hz, 1H), 6.28 (d, J=8.9 Hz, 1H), 3.79-3.52 (m, 6H), 2.43-2.26 (m, 4H). MS (M+H)¹=432.1.

### Example 131A—Synthesis of 371A

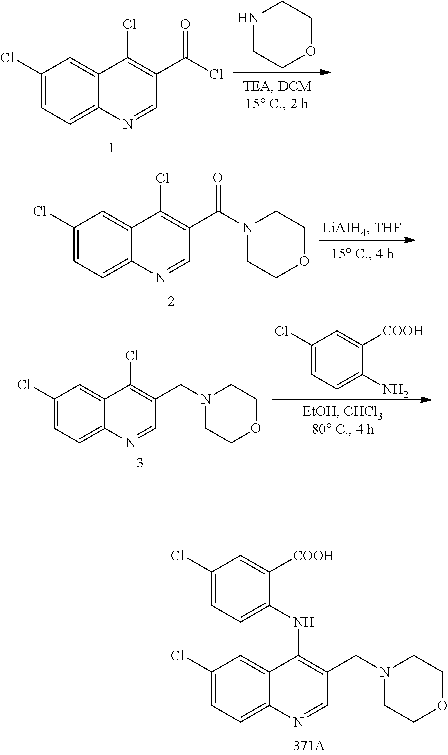

### Synthesis of (4,6-dichloro-3-quinolyl)-morpholino-methanone (2)

To a solution of 4,6-dichloroquinoline-3-carbonyl chloride (2.5 g, 9.60 mmol, 1 eq) in DCM (20 mL) was added TEA (2.91 g, 28.79 mmol, 4.01 mL, 3 eq) and morpholine (836.07 mg, 9.60 mmol, 844.52 uL, 1 eq) at 0° C., the reaction was stirred at 20° C. for 2 h under N₂. LCMS showed starting material was consumed completely and the MS of desired product was detected. The reaction was cooled to ambient temperature, quenched with water (20 ml) and extracted with ethyl acetate (20 m). The organic layer was washed with water, brine, dried over anhydrous sodium sulfate, filtered and concentrated in vacuum. Compound (4,6-dichloro-3-quinolyl)-morpholino-methanone (2.8 g, 9.00 mmol, 93.77% yield) was obtained as a brown solid. MS (M+H)⁺=311.1.

### Synthesis of 4-[(4,6-dichloro-3-quinolyl)methyl]morpholine (3)

To a solution of (4,6-dichloro-3-quinolyl)-morpholino-methanone (500 mg, 1.61 mmol, 1 eq) in THF (8 mL) was added LiAlH4 (60.98 mg, 1.61 mmol, 1 eq), the mixture was purged with N₂, the reaction was stirred at 15° C. for 4 h under N₂. LCMS showed starting material was consumed completely and the MS of desired product was detected. The reaction mixture was concentrated in vacuum. The crude product was purified by prep-HPLC (column: Phenomenex luna C18 250*50 mm*10 um; mobile phase:[water(0.04% HCl)-ACN]; B %: 15%-45%,10 min) Compound 4-[(4,6-dichloro-3-quinolyl)methyl]morpholine (100 mg, 336.50 umol, 20.94% yield) was obtained as a white solid. MS (M+H)⁺=297.0.

### Synthesis of 5-chloro-2-[[6-chloro-3-(morpholinomethyl)-4-quinolyl]amino]benzoic acid (371A)

To a solution of 4-[(4,6-dichloro-3-quinolyl)methyl]morpholine (60 mg, 201.90 umol, 1 eq) in EtOH (1 mL) and CHCl₃ (0.3 mL) was added 2-amino-5-chloro-benzoic acid (34.64 mg, 201.90 umol, 1 eq), the reaction was stirred at 80° C. for 12 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 10%-35%,8 min) Compound 5-chloro-2-[[6-chloro-3-(morpholinomethyl)-4-quinolyl]amino]benzoic acid (12.2 mg, 25.37 umol, 12.57% yield, 97.48% purity, HCl) was obtained as a yellow solid. ¹H NMR (400 MHz, DMSO-d₆) δ=9.28 (s, 1H), 8.20 (d, J=9.0 Hz, 1H), 8.01-7.92 (m, 2H), 7.70 (d, J=2.0 Hz, 1H), 7.54 (dd, J=2.4, 8.8 Hz, 1H), 6.93 (br d, J=6.6 Hz, 1H), 4.64-4.46 (m, 2H), 3.86 (br s, 4H), 3.41-3.06 (m, 4H). MS (M+H)⁺=432.0.

### Example 132—Synthesis of 372A

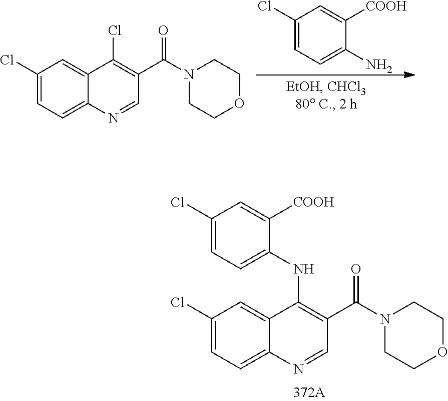 [SMILES: O=C(c1cnc2ccc(Cl)cc2c1Cl)N1CCOCC1 ; Nc1ccc(Cl)cc1C(=O)O ; O=C(O)c1cc(Cl)ccc1Nc1c(C(=O)N2CCOCC2)cnc2ccc(Cl)cc12]

### Synthesis of 5-chloro-2-[[6-chloro-3-(morpholine-4-carbonyl)-4-quinolyl]amino]benzoic acid (372A)

To a solution of 2-amino-5-chloro-benzoic acid (44.11 mg, 257.10 umol, 1 eq) in EtOH (1 mL) and CHCl₃ (0.3 mL) was added (4,6-dichloro-3-quinolyl)-morpholino-methanone (80 mg, 257.10 umol, 1 eq), the reaction was stirred at 80° C. for 2 h. LCMS showed starting material was consumed completely and the MS of desired product was detected. The reaction mixture was concentrated in vacuum The crude product was purified by prep-HPLC (column: Waters Xbridge Prep OBD C18 150*40 mm*10 um; mobile phase:[water(NH₃H₂O+NH₄HCO₃)-ACN]; B %: 10%-30%,8 min) Compound 5-chloro-2-[[6-chloro-3-(morpholine-4-carbonyl)-4-quinolyl]amino]benzoic acid (22 mg, 49.01 umol, 19.06% yield, 99.42% purity) was obtained as a yellow solid. ¹H NMR (400 MHz, DMSO-d₆+D₂O) δ=8.67 (s, 1H), 8.09-8.00 (m, 2H), 7.88-7.80 (m, 2H), 7.28 (dd, J=2.5, 8.8 Hz, 1H), 6.69 (d, J=8.9 Hz, 1H), 3.47-3.30 (m, 4H), 3.29-3.02 (m, 4H). MS (M+H)⁺=446.1.

### Example 133—Synthesis of 373A

Synthetic scheme is shown in FIG. 39I.

### Synthesis of 6-chloro-3-morpholinosulfonyl-quinolin-4-ol (2)

To a solution of 6-chloro-4-hydroxy-quinoline-3-sulfonyl chloride (3 g, 10.79 mmol, 1 eq) in DCM (15 mL) was added TEA (3.27 g, 32.36 mmol, 4.50 mL, 3 eq) and morpholine (939.77 mg, 10.79 mmol, 949.26 uL, 1 eq) at 0° C., the reaction was stirred at 15° C. for 2 h. LCMS showed starting material was consumed completely and the MS of desired product was detected. The reaction mixture was concentrated in vacuum. Compound 6-chloro-3-morpholinosulfonyl-quinolin-4-ol (2.5 g, 7.60 mmol, 70.49% yield) was obtained as a white oil. MS (M+H)⁺=219.1.

### Synthesis of 4-[(4,6-dichloro-3-quinolyl)sulfonyl]morpholine (3)

A solution of 6-chloro-3-morpholinosulfonyl-quinolin-4-ol (2.5 g, 7.60 mmol, 1 eq) in POCl₃ (15 mL), the mixture was purged with N₂, the reaction was stirred at 100° C. for 12 h under N₂. LCMS showed starting material was consumed completely and the MS of desired product was detected. The reaction was cooled to ambient temperature, the mixture was concentrated in vacuum, then the mixture was added extracted with ethyl acetate (20 ml) and H₂O (30 ml). The organic layer was washed with brine, dried over anhydrous sodium sulfate, filtered and concentrated in vacuum. The residue was purified by flash column (ISCO 40 g silica, 10-40% ethyl acetate in petroleum ether, gradient over 20 min). TLC (PE:EtOAc=2:1,R_{f}=0.52) Compound 4-[(4,6-dichloro-3-quinolyl)sulfonyl]morpholine (550 mg, 1.58 mmol, 20.83% yield) was obtained as a white solid. MS (M+H)⁺=347.0.

### Synthesis of 4-[(6-chloro-3-morpholinosulfonyl-4-quinolyl)amino]-3-methoxycarbonyl-benzoic acid (4)

To a solution of 4-amino-3-methoxycarbonyl-benzoic acid (150 mg, 768.55 umol, 1 eq) in THF (4 mL) was added LiHMDS (1 M, 1.15 mL, 1.5 eq) dropwise, the mixture was stirred at 15° C. for 0.5 h under N₂, then the mixture was added 4-[(4,6-dichloro-3-quinolyl)sulfonyl]morpholine (266.85 mg, 768.55 umol, 1 eq), the reaction was stirred at 15° C. for 4 h under N₂. LCMS showed starting material was consumed completely and the MS of desired product was detected. The reaction mixture was concentrated in vacuum. The crude product was purified by prep-HPLC (column: Phenomenex luna C18 80*40 mm*3 um; mobile phase: [water(0.04% HCl)-ACN]; B %: 32%-52%,7 min) Compound 4-[(6-chloro-3-morpholinosulfonyl-4-quinolyl)amino]-3-methoxycarbonyl-benzoic acid (60 mg, 118.59 umol, 15.43% yield) was obtained as a yellow solid. MS (M+H)⁺=506.1.

### Synthesis of methyl 5-carbamoyl-2-[(6-chloro-3-morpholinosulfonyl-4-quinolyl)amino]benzoate (5)

To a solution of 4-[(6-chloro-3-morpholinosulfonyl-4-quinolyl)amino]-3-methoxycarbonyl-benzoic acid (35 mg, 69.18 umol, 1 eq) in DMF (0.5 mL) was added HATU (39.46 mg, 103.77 umol, 1.5 eq), DIPEA (26.82 mg, 207.54 umol, 36.15 uL, 3 eq) and NH₄Cl (11.10 mg, 207.54 umol, 3 eq), the reaction was stirred at 15° C. for 2 h. LCMS showed starting material was consumed completely and the MS of desired product was detected. The reaction mixture was concentrated in vacuum. Compound methyl 5-carbamoyl-2-[(6-chloro-3-morpholinosulfonyl-4-quinolyl)amino]benzoate (30 mg, 59.41 umol, 85.88% yield) was obtained as a yellow solid. MS (M+H)⁺=505.1.

### Synthesis of methyl 5-carbamoyl-2-[(6-chloro-3-morpholinosulfonyl-4-quinolyl)amino]benzoic acid (373A)

To a solution of methyl 5-carbamoyl-2-[(6-chloro-3-morpholinosulfonyl-4-quinolyl)amino]benzoate (30 mg, 59.41 umol, 1 eq) in THF (0.5 mL), MeOH (0.1 mL) and H₂O (0.1 mL) was added LiOH·H₂O (4.99 mg, 118.83 umol, 2 eq), the reaction was stirred at 60° C. for 2 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 30%-65%,8 min), afford crude product 10 mg. The crude product was purified by prep-HPLC (column: PhenomenexLuna C18 150*30 mm*5 um; mobile phase: [water(0.04% TFA)-ACN]; B %: 30%-55%,8 min) Compound 5-carbamoyl-2-[(6-chloro-3-morpholinosulfonyl-4-quinolyl)amino]benzoic acid (3.20 mg, 5.22 umol, 8.79% yield, 98.71% purity, TFA) was obtained as a yellow solid. ¹H NMR (400 MHz, DMSO-d₆+D₂O) δ=9.12 (s, 1H), 8.55 (d, J=2.0 Hz, 1H), 8.17 (d, J=8.9 Hz, 1H), 7.93 (dd, J=2.1, 8.9 Hz, 1H), 7.78 (br d, J=8.8 Hz, 1H), 7.65 (s, 1H), 6.62 (d, J=8.6 Hz, 1H), 3.50-3.39 (m, 2H), 3.37-3.25 (m, 2H), 3.11-2.92 (m, 4H). MS (M+H)⁺=490.9.

### Example 134—Synthesis of 376A

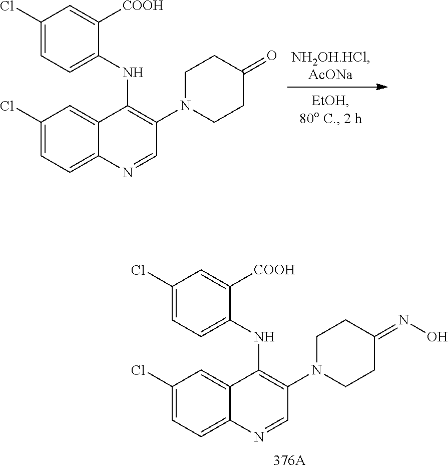 [SMILES: O=C1CCN(c2cnc3ccc(Cl)cc3c2Nc2ccc(Cl)cc2C(=O)O)CC1 ; O=C(O)c1cc(Cl)ccc1Nc1c(N2CCC(=NO)CC2)cnc2ccc(Cl)cc12]

### Synthesis of 5-chloro-2-[[6-chloro-3-(4-hydroxyimino-1-piperidyl)-4-quinolyl]amino]benzoic acid (376A)

To a solution of 5-chloro-2-[[6-chloro-3-(4-oxo-1-piperidyl)-4-quinolyl]amino]benzoic acid (6 mg, 13.94 umol, 1 eq) in EtOH (0.5 mL) was added AcONa (1.72 mg, 20.92 umol, 1.5 eq) and hydroxylamine; hydrochloride (1.45 mg, 20.92 umol, 1.5 eq), the reaction was stirred at 80° C. for 2 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 20%-60%,8 min) Compound 5-chloro-2-[[6-chloro-3-(4-hydroxyimino-1-piperidyl)-4-quinolyl]amino]benzoic acid (2.4 mg, 5.39 umol, 38.65% yield, 100% purity) was obtained as a yellow solid. ¹H NMR (400 MHz, DMSO-d₆) δ=10.54-10.29 (m, 2H), 8.80 (s, 1H), 8.45 (br s, 1H), 8.14 (d, J=9.0 Hz, 1H), 8.01-7.83 (m, 2H), 7.61 (dd, J=2.4, 8.7 Hz, 1H), 7.28-7.05 (m, 1H), 3.03-2.83 (m, 4H), 2.12 (br s, 2H), 1.89 (br t, J=5.1 Hz, 2H). MS (M+H)⁺=445.0.

### Example 135—Synthesis of 391A

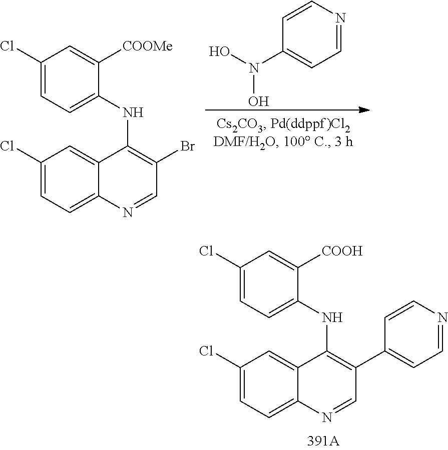 [SMILES: COC(=O)c1cc(Cl)ccc1Nc1c(Br)cnc2ccc(Cl)cc12 ; ON(O)c1ccncc1 ; O=C(O)c1cc(Cl)ccc1Nc1c(-c2ccncc2)cnc2ccc(Cl)cc12]

### Synthesis of 5-chloro-2-[[6-chloro-3-(4-pyridyl)-4-quinolyl]amino]benzoic acid (391A)

To a stirred solution of methyl 2-[(3-bromo-6-chloro-4-quinolyl)amino]-5-chloro-benzoate (100 mg, 234.69 umol, 1 eq) in DMF (4 mL) and H₂O (1 mL) was added 4-pyridylboronic acid (31.73 mg, 258.16 umol, 1.1 eq), Pd(dppf)Cl₂ (17.17 mg, 23.47 umol, 0.1 eq) and Cs₂CO₃ (229.40 mg, 704.07 umol, 3 eq), the mixture was bubbled with N₂ for 1 minute, and stirred at 100° C. for 3 h. LCMS showed the starting material was consumed completely and desired product was detected. The reaction mixture was filtered, and filtrate was purified by prep-HPLC (column: Waters Xbridge Prep OBD C18 150*40 mm*10 um; mobile phase: [water(NH₄HCO₃)-ACN]; B %: 20%-50%,8 min). Compound 5-chloro-2-[[6-chloro-3-(4-pyridyl)-4-quinolyl]amino]benzoic acid (7.4 mg, 15.81 umol, 6.74% yield, 95.42% purity, HCl) was obtained as a yellow solid. ¹H NMR (400 MHz, DMSO-d6) δ ppm 10.46-10.98 (m, 1H), 8.94 (s, 1H), 8.86 (br d, J=14.88 Hz, 1H), 8.68 (br d, J=4.00 Hz, 2H), 8.30 (br t, J=7.82 Hz, 1H), 8.12 (br d, J=8.88 Hz, 1H), 7.77 (br s, 2H), 7.67 (s, 1H), 7.23 (br d, J=8.63 Hz, 1H), 6.91 (br t, J=9.38 Hz, 1H). MS (M+H)⁺=410.0.

### Example 136—Synthesis of 392A

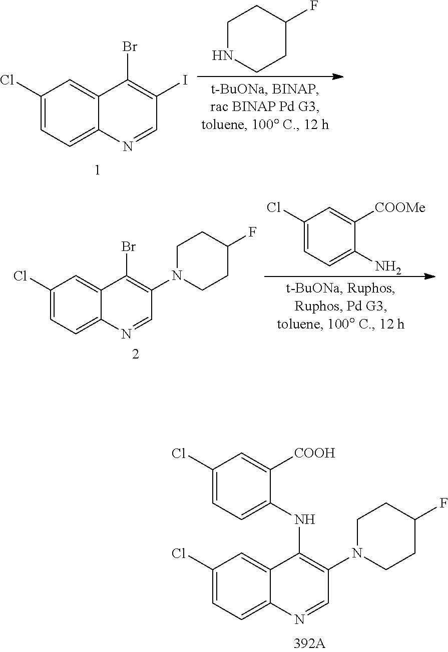

### Synthesis of 4-bromo-6-chloro-3-(4-fluoro-1-piperidyl)quinoline (2)

To a solution of 4-bromo-6-chloro-3-iodo-quinoline (300 mg, 814.34 umol, 1 eq) in toluene (1 mL) was added BINAP (50.71 mg, 81.43 umol, 0.1 eq), t-BuONa (234.78 mg, 2.44 mmol, 3 eq), rac binap pd G3 (80.71 mg, 81.43 umol, 0.1 eq) and 4-fluoropiperidine (83.99 mg, 814.34 umol, 1 eq), the reaction was purged with N₂, the reaction was stirred at 100° C. for 12 h. LCMS showed starting material was consumed completely and the MS of desired product was detected. The reaction was cooled to ambient temperature, quenched with water (10 ml) and extracted with ethyl acetate (10 ml). The organic layer was washed with water, brine, dried over anhydrous sodium sulfate, filtered and concentrated in vacuum. The residue was purified by flash column (ISCO 10 g silica, 10-30% ethyl acetate in petroleum ether, gradient over 20 min). TLC (Petroleum ether/Ethyl acetate=3:1, R_{f}=0.51) Compound 4-bromo-6-chloro-3-(4-fluoro-1-piperidyl)quinoline (180 mg, 523.83 umol, 64.33% yield) was obtained as a white solid. MS (M+H)⁺=345.1.

### Synthesis of 5-chloro-2-[[6-chloro-3-(4-fluoro-1-piperidyl)-4-quinolyl]amino]benzoic acid (392A)

To a solution of 4-bromo-6-chloro-3-(4-fluoro-1-piperidyl)quinoline (100 mg, 291.02 umol, 1 eq) in toluene (2 mL) was added t-BuONa (83.90 mg, 873.05 umol, 3 eq), RuPhos Pd G3 (24.34 mg, 29.10 umol, 0.1 eq), RuPhos (13.58 mg, 29.10 umol, 0.1 eq) and methyl 2-amino-5-chloro-benzoate (54.02 mg, 291.02 umol, 1 eq), the mixture was purged with N₂, the reaction was stirred at 100° C. for 12 h under N₂. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 20%-40%,8 min) Compound 5-chloro-2-[[6-chloro-3-(4-fluoro-1-piperidyl)-4-quinolyl]amino]benzoic acid (24.5 mg, 51.36 umol, 17.65% yield, 98.69% purity, HCl) was obtained as a yellow solid. ¹H NMR (400 MHz, DMSO-d₆+D₂O) δ=8.74 (s, 1H), 8.30 (s, 1H), 8.02 (d, J=9.1 Hz, 1H), 7.91-7.83 (m, 2H), 7.55 (dd, J=2.5, 8.8 Hz, 1H), 7.03 (d, J=8.9 Hz, 1H), 4.75-4.49 (m, 1H), 3.06-2.93 (m, 2H), 2.85-2.72 (m, 2H), 1.60-1.42 (m, 2H), 1.40-1.25 (m, 2H). MS (M+H)⁺=434.0.

### Example 137—Synthesis of 393A

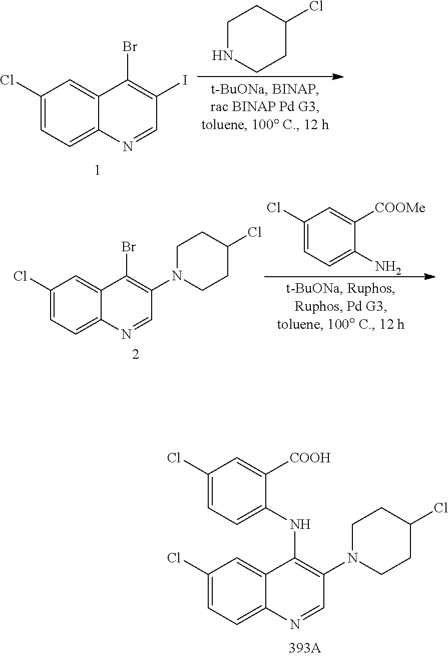

### 1. Synthesis of 4-bromo-6-chloro-3-(4-chloro-1-piperidyl)quinoline (2)

To a solution of 4-bromo-6-chloro-3-iodo-quinoline (300 mg, 814.34 umol, 1 eq) in toluene (1 mL) was added t-BuONa (234.78 mg, 2.44 mmol, 3 eq), BINAP (50.71 mg, 81.43 umol, 0.1 eq), rac BINAP Pd G3 (80.72 mg, 81.43 umol, 0.1 eq) and 4-chloropiperidine; hydrochloride (127.08 mg, 814.34 umol, 1 eq), the mixture was purged with N₂, the reaction was stirred at 100° C. for 12 h under N₂. LCMS showed starting material was consumed completely and the MS of desired product was detected. The reaction was cooled to ambient temperature, quenched with water (10 ml) and extracted with ethyl acetate (10 ml). The organic layer was washed with water, brine, dried over anhydrous sodium sulfate, filtered and concentrated in vacuum. The residue was purified by flash column (ISCO 20 g silica, 40-60% ethyl acetate in petroleum ether, gradient over 20 min). TLC (Petroleum ether/Ethyl acetate=1:1, R_{f}=0.40) Compound 4-bromo-6-chloro-3-(4-chloro-1-piperidyl)quinoline (140 mg, 388.81 umol, 47.74% yield) was obtained as a pale yellow solid. MS (M+H)⁺=361.0.

### Synthesis of 5-chloro-2-((6-chloro-3-(4-chloropiperidin-1-yl)quinolin-4-yl)amino)benzoic acid (393A)

To a solution of methyl 2-amino-5-chloro-benzoate (41.24 mg, 222.18 umol, 1 eq) in toluene (4 mL) was added t-BuONa(64.06 mg, 666.53 umol, 3 eq), RuPhos (10.37 mg, 22.22 umol, 0.1 eq), RuPhos Pd G3 (18.58 mg, 22.22 umol, 0.1 eq) and 4-bromo-6-chloro-3-(4-chloro-1-piperidyl) quinoline (80 mg, 222.18 umol, 1 eq), the mixture was purged with N₂, the reaction was stirred at 100° C. for 12 h under N₂. LCMS showed starting material was consumed completely and the MS of desired product was detected. The reaction mixture was concentrated under reduced pressure to give a residue. The residue was purified by prep-HPLC (column: Phenomenex Luna 80*30 mm*3 um; mobile phase: [water(0.04% HCl)-ACN]; B %: 20%-55%,8 min). Afford 5 mg crude product. The crude product was purified by prep-HPLC (column: Phenomenex C18 75*30 mm*3 um; mobile phase:[water(NH₃H₂O+NH₄HCO₃)-ACN]; B %:10%-50%,8 min). Compound 5-chloro-2-[[6-chloro-3-(4-chloro-1-piperidyl)-4-quinolyl]amino]benzoic acid (2.4 mg, 5.32 umol, 2.40% yield) was obtained as a yellow solid. ¹H NMR (400 MHz, DMSO-d₆) δ=8.73 (s, 1H), 7.97 (d, J=9.0 Hz, 1H), 7.86 (d, J=2.5 Hz, 1H), 7.76 (d, J=2.1 Hz, 1H), 7.62 (dd, J=2.2, 8.9 Hz, 1H), 7.33 (dd, J=2.6, 8.8 Hz, 1H), 6.43 (d, J=8.8 Hz, 1H), 4.25-4.14 (m, 1H), 3.27-3.17 (m, 2H), 3.01-2.87 (m, 2H), 1.91 (br dd, J=2.3, 9.4 Hz, 2H), 1.65-1.33 (m, 2H). MS (M+H)⁺=450.1.

### Example 138—Synthesis of 394A

Synthetic details are provided in FIG. 39J.

### Synthesis of tert-butyl 4-(trifluoromethoxy)piperidine-1-carboxylate (3F)

To a reaction flask that was equipped with a stirring bar, silver; trifluoromethanesulfonate (3.83 g, 14.91 mmol, 3 eq), 1-(chloromethyl)-4-fluoro-1,4-diazoniabicyclo[2.2.2]octane; ditetrafluoroborate (2.64 g, 7.45 mmol, 1.5 eq), fluoropotassium (1.15 g, 19.87 mmol, 465.58 uL, 4 eq), tert-butyl 4-hydroxypiperidine-1-carboxylate (1 g, 4.97 mmol, 1 eq) were added successively in the nitrogen atmosphere. Then ethyl acetate (25 mL), 2-fluoropyridine (1.45 g, 14.91 mmol, 1.28 mL, 3 eq) and trimethyl(trifluoromethyl)silane (2.12 g, 14.91 mmol, 3 eq) were added successively under nitrogen atmosphere. The reaction mixture was stirred at 25° C. for 12 h. After 12 hours, LCMS analysis showed that starting material and target compound failed to be detected. And TLC (Petroleum ether:Ethyl acetate=5:1, stained by iodine) indicated that a major spot (R_{f}=0.7) was detected. The reaction mixture was filtered through a plug of silica (eluted with ethyl acetate) to remove the precipitate, then the filtrate was concentrated to give the crude product. The product was purified by column chromatography on silica gel (ISCO; 40 g SepaFlash Silica Flash Column, Eluent of 3˜5% Ethyl acetate/Petroleum ether gradient at 120 m/min). Product tert-butyl 4-(trifluoromethoxy)piperidine-1-carboxylate (265 mg, 984.18 umol, 19.81% yield) was obtained as colorless oil. ¹H NMR (400 MHz, CDCl₃) δ ppm 4.44-4.40 (m, 1H), 3.72-3.69 (m, 2H), 3.32-3.26 (m, 2H), 1.90-1.74 (m, 4H), 6 1.47 (s, 9H).

### Synthesis of 4-(trifluoromethoxy)piperidine (3D)

To a three-neck flask was placed with tert-butyl 4-(trifluoromethoxy)piperidine-1-carboxylate (170 mg, 631.36 umol, 1 eq) in ethyl acetate (1.5 mL), then hydrogen chloride gas (4 M, 2 mL, 12.67 eq) in ethyl acetate was added. The above solution was stirred at 25° C. for 1.5 hours. TLC showed that the starting material was consumed completely in 1.5 hours. The original solution was concentrated in vacuum to give the white solid directly and the resultant mixture was dissolved in 12 mL ethanol and basified by adding ion exchange resin, then filtered to remove the resin, and the filtrate was concentrated in reduced pressure. The desired product 4-(trifluoromethoxy)piperidine (93.5 mg, 552.78 umol, 87.55% yield) was obtained as pale yellow solid. ¹H NMR (400 MHz, CDCl₃) δ ppm 9.49 (brs, 1H), 4.6 (m, 1H), 3.31-3.29 (m, 4H), 2.38-2.29 (m, 2H), 2.17-2.12 (m, 2H).

### Synthesis of 4-bromo-6-chloro-3-[4-(trifluoromethoxy)-1-piperidyl]quinoline (2)

A mixture of 4-bromo-6-chloro-3-iodo-quinoline (125 mg, 339.31 umol, 1 eq), 4-(trifluoromethoxy)piperidine (63.13 mg, 373.24 umol, 1.1 eq), sodium; 2-methylpropan-2-olate (97.83 mg, 1.02 mmol, 3 eq), rac-BINAP-Pd-G3 (33.67 mg, 33.93 umol, 0.1 eq) and [1-(2-diphenylphosphanyl-1-naphthyl)-2-naphthyl]-diphenyl-phosphane (21.13 mg, 33.93 umol, 0.1 eq) in toluene (2 mL) was degassed and purged with N₂ for three times, and then the mixture was stirred at 100° C. for 12 hours under N₂ atmosphere. LCMS showed that the desired mass was detected, and starting material still remained even with prolonged time. The mixture was concentrated in reduced pressure to give the crude product. The residue was purified by flash silica gel chromatography (ISCO; 20 g SepaFlash Silica Flash Column, Eluent of 5˜20% Ethyl acetate/Petroleum ether gradient at 50 mL/min). Compound 4-bromo-6-chloro-3-[4-(trifluoromethoxy)-1-piperidyl]quinoline (33.0 mg, 80.56 umol, 23.74% yield) was obtained as yellow solid. ¹H NMR (400 MHz, CDCl₃) δ ppm 8.68 (s, 1H). 8.20 (d, J=2.4 Hz, 1H). 7.98 (d, J=8.8 Hz, 1H). 7.57 (dd, J=8.8, 2.4 Hz, 1H), 4.56-4.50 (m, 1H), 3.49-3.43 (m, 2H), 3.21-3.15 (m, 2H), 2.23-2.16 (m, 2H), 2.14-2.05 (m, 2H). MS (M+H)⁺=409.0.

### Synthesis of 5-chloro-2-[[6-chloro-3-[4-(trifluoromethoxy)-1-piperidyl]-4-quinolyl]amino]benzoic acid (394A)

The mixture of 4-bromo-6-chloro-3-[4-(trifluoromethoxy)-1-piperidyl]quinoline (33.0 mg, 80.56 umol, 1 eq), methyl 2-amino-5-chloro-benzoate (17.94 mg, 96.67 umol, 1.2 eq), rac-BINAP-Pd-G3 (7.99 mg, 8.06 umol, 0.1 eq) and Cs₂CO₃ (52.50 mg, 161.12 umol, 2 eq) in tert amyl alcohol (1.5 mL) was heated at 100° C. in the N₂ atmosphere for 12 hours. LCMS showed that starting material has been consumed completely, and the desired mass peak was detected as majority. The mixture was concentrated in the reduced pressure. The crude compound was purified by flash silica gel chromatography (ISCO; 10 g SepaFlash Silica Flash Column, eluent of 5˜50% Ethyl acetate/Petroleum ether, then 2˜7% Methanol/Dichloromethane gradient at 50 mL/min) to give the isolated compound. Finally, the crude product was purified by HPLC (column: Phenomenex Luna C18 75*30 mm*3 um; mobile phase: [water(TFA)-ACN]; B %: 40%-70%, 8 min) to give 5-chloro-2-[[6-chloro-3-[4-(trifluoromethoxy)-1-piperidyl]-4-quinolyl]amino]benzoic acid as yellow solid (13.0 mg, 25.72 μmol, 31.93%, 99% purity). ¹H NMR (400 MHz, CDCl₃) δ=10.59 (s, 1H), 8.82 (s, 1H), 8.32 (d, J=8.0 Hz, 1H), 8.12 (s, 1H), 7.81-7.74 (m, 2H), 7.40 (d, J=8.0 Hz, 1H), 6.75 (d, J=8.8 Hz, 1H), 4.35 (m, 1H), 3.16-3.12 (m, 2H), 2.89-2.86 (m, 2H), 1.79-1.70 (m, 4H). MS (M+H)⁺=500.1.

### Example 139—Synthesis of 395A

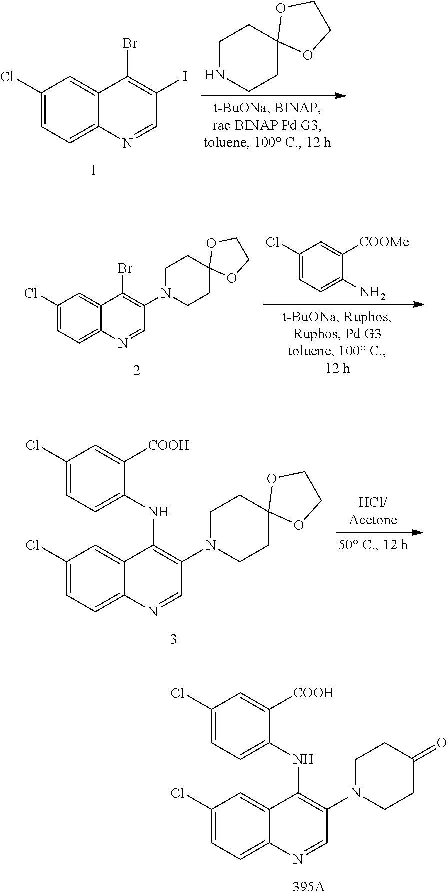

### Synthesis of 8-(4-bromo-6-chloro-3-quinolyl)-1,4-dioxa-8-azaspiro[4.5]decane (2)

To a solution of 4-bromo-6-chloro-3-iodo-quinoline (200 mg, 542.89 umol, 1 eq) in toluene (3 mL) was added t-BuONa (156.52 mg, 1.63 mmol, 3 eq), BINAP (33.80 mg, 54.29 umol, 0.1 eq), [2-(2-aminophenyl)phenyl]-methylsulfonyloxypalladium; [1-(2-diphenylphosphanyl-1-naphthyl)-2-naphthyl]-diphenyl-phosphane (53.88 mg, 54.29 umol, 0.1 eq) and 1,4-dioxa-8-azaspiro[4.5]decane (77.73 mg, 542.89 umol, 69.40 uL, 1 eq), the reaction was stirred at 100° C. for 12 h under Ar. LCMS showed starting material was consumed completely and the MS of desired product was detected. The reaction was cooled to ambient temperature, quenched with water (20 ml) and extracted with ethyl acetate (20 ml). The organic layer was washed with water, brine, dried over anhydrous sodium sulfate, filtered and concentrated in vacuo. The residue was purified by flash column (ISCO 20 g silica, 10-40% ethyl acetate in petroleum ether, gradient over 20 min). TLC (Petroleum ether/Ethyl acetate=2:1, R_{f}=0.50) Compound 8-(4-bromo-6-chloro-3-quinolyl)-1,4-dioxa-8-azaspiro[4.5]decane (180 mg, 469.16 umol, 86.42% yield) was obtained as a white solid. MS (M+H)⁺=385.1.

### Synthesis of 5-chloro-2-[[6-chloro-3-(1,4-dioxa-8-azaspiro[4.5]decan-8-yl)-4-quinolyl]amino]benzoic acid (3)

To a solution of 8-(4-bromo-6-chloro-3-quinolyl)-1,4-dioxa-8-azaspiro[4.5]decane (140 mg, 364.90 umol, 1 eq) in toluene (1 mL) was added NaOBu-t (105.20 mg, 1.09 mmol, 3 eq), RuPhos (17.03 mg, 36.49 umol, 0.1 eq), RuPhos Pd G3 (305.19 mg, 364.90 umol, 1 eq) and methyl 2-amino-5-chloro-benzoate (67.73 mg, 364.90 umol, 1 eq), the reaction was stirred at 100° C. for 12 h under Ar. LCMS showed starting material was consumed completely and the MS of desired product was detected. The reaction mixture was concentrated in vacuum. The crude product was purified by prep-HPLC (column: Waters Xbridge Prep OBD C18 150*40 mm*10 um; mobile phase: [water(10 mmol NH₄HCO₃)-ACN]; B %: 20%-50%,8 min) Compound 5-chloro-2-[[6-chloro-3-(1,4-dioxa-8-azaspiro[4.5]decan-8-yl)-4-quinolyl]amino]benzoic acid (40 mg, 84.33 umol,23.11% yield) was obtained as a yellow solid. MS (M+H)⁺=474.1.

### Synthesis of 5-chloro-2-[[6-chloro-3-(4-oxo-1-piperidyl)-4-quinolyl]amino]benzoic acid (395A)

A solution of 5-chloro-2-[[6-chloro-3-(1,4-dioxa-8-azaspiro[4.5]decan-8-yl)-4-quinolyl]amino]benzoic acid (35 mg, 73.79 umol, 1 eq) in ACETONE (0.2 mL) and HCl (3 M, 3.50 mL, 142.30 eq), the mixture was stirred at 70° C. for 1 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 1%-60%,8 min) Compound 5-chloro-2-[[6-chloro-3-(4-oxo-1-piperidyl)-4-quinolyl]amino]benzoic acid (9.9 mg, 21.21 umol, 28.75% yield,100% purity, HCl) was obtained as a yellow solid. ¹H NMR (400 MHz, DMSO-d₆) δ=10.49-10.20 (m, 1H), 8.96-8.75 (m, 1H), 8.50-8.26 (m, 1H), 8.24-8.09 (m, 1H), 8.01-7.80 (m, 2H), 7.66-7.47 (m, 1H), 7.26-6.94 (m, 1H), 3.22 (br s, 4H), 2.06 (br s, 4H). MS (M+H)⁺*=429.9

### Example 140—Synthesis of 396A

Synthetic scheme is shown in FIG. 39K.

### (1,3-dioxoisoindolin-2-yl) 1,4-dioxaspiro[4.5]decane-8-carboxylate (2)

To a solution of 1,4-dioxaspiro[4.5]decane-8-carboxylic acid (3.7 g, 19.87 mmol, 1 eq) in DCM (40 mL) was added 2-hydroxyisoindoline-1,3-dione (3.24 g, 19.87 mmol, 1 eq), EDCI (4.57 g, 23.84 mmol, 1.2 eq), DMAP (728.27 mg, 5.96 mmol, 0.3 eq), the mixture was stirred at 25° C. for 1 hr. LCMS showed the reaction was complete. 30 mL of Water was added to the reaction, the reaction mixture was extracted with DCM (60 mL). The combined organic layers were washed with brine, dried over anhydrous sodium sulfate and filtered. The filtrate was concentrated to dryness to give residue. The residue was purified by flash silica gel chromatography (ISCO; 40 g SepaFlash Silica Flash Column, Eluent of 5˜30% Ethyl acetate/Petroleum ether gradient @100 mL/min). Compound (1,3-dioxoisoindolin-2-yl) 1,4-dioxaspiro[4.5]decane-8-carboxylate (5.7 g, 17.20 mmol, 86.58% yield) was obtained as a white solid. MS (M+H)⁺=332.1.

### Methyl 5-chloro-2-[[6-chloro-3-(1,4-dioxaspiro[4.5]decan-8-yl)-4-quinolyl]amino]benzoate (3)

To a solution of methyl 2-[(3-bromo-6-chloro-4-quinolyl)amino]-5-chloro-benzoate (500.00 mg, 1.17 mmol, 1 eq), (1,3-dioxoisoindolin-2-yl) 1,4-dioxaspiro[4.5]decane-8-carboxylate (388.79 mg, 1.17 mmol, 1 eq) and Zn (153.46 mg, 2.35 mmol, 2 eq) in DMA (2 mL) was added Ni(dtbbpy)Br₂ (114.25 mg, 234.69 umol, 0.2 eq). The mixture was stirred at 40° C. for 12 hr under N₂. LCMS showed the reaction was complete. The reaction was cooled to ambient temperature, 10 mL of water was added to the reaction and the reaction mixture was extracted with Ethyl acetate (30 mL). The combined organic layers were washed with brine (10 mL) and dried over anhydrous sodium sulfate. The combined organic layer was concentrated to dryness to give residue. The residue was purified by flash silica gel chromatography (ISCO; 12 g SepaFlash Silica Flash Column, Eluent of 15˜35% Ethyl acetate/Petroleum ether gradient a 80 mL/min). Then the crude product was purified by Prep-TLC (Ethyl acetate/Petroleum ether=3/1). Compound methyl 5-chloro-2-[[6-chloro-3-(1,4-dioxaspiro[4.5]decan-8-yl)-4-quinolyl]amino]benzoate (10 mg, 20.52 umol, 1.75% yield) was obtained as a yellow oil. MS (M+H)⁺=487.1.

### 5-chloro-2-[[6-chloro-3-(1,4-dioxaspiro[4.5]decan-8-yl)-4-quinolyl]amino]benzoic acid (4)

To a solution of methyl 5-chloro-2-[[6-chloro-3-(1,4-dioxaspiro[4.5]decan-8-yl)-4-quinolyl]amino]benzoate (10 mg, 20.52 umol, 1 eq) in THF (1 mL), MeOH (0.2 mL) and H₂O (0.2 mL) was added LiOH·H₂O (1.72 mg, 41.04 umol, 2 eq), the mixture was stirred at 60° C. for 1 hr. LCMS showed the reaction was complete. The reaction mixture was concentrated in vacuum. Compound 5-chloro-2-[[6-chloro-3-(1,4-dioxaspiro[4.5]decan-8-yl)-4-quinolyl]amino]benzoic acid (10 mg, crude) was obtained as a yellow solid. MS (M+H)⁺=473.1.

### 5-chloro-2-[[6-chloro-3-(4-oxocyclohexyl)-4-quinolyl]amino]benzoic acid (5)

To a solution of 5-chloro-2-[[6-chloro-3-(1,4-dioxaspiro[4.5]decan-8-yl)-4-quinolyl]amino]benzoic acid (10 mg, 21.13 umol, 1 eq) in acetone (0.7 mL) was added HCl (0.3 M, 0.3 mL, 4.26 eq), the mixture was stirred at 70° C. for 1 hr. LCMS showed the reaction was complete. The reaction mixture was concentrated in vacuum. Compound 5-chloro-2-[[6-chloro-3-(4-oxocyclohexyl)-4-quinolyl]amino]benzoic acid (10 mg, crude, HCl salt) was obtained as a yellow solid. MS (M+H)⁺=429.0.

### 5-chloro-2-[[6-chloro-3-(4-hydroxyiminocyclohexyl)-4-quinolyl]amino]benzoic acid (396A)

To a solution of 5-chloro-2-[[6-chloro-3-(4-oxocyclohexyl)-4-quinolyl]amino]benzoic acid (10 mg, 23.29 umol, 1 eq) in EtOH (1 mL) was added NaOAc (2.87 mg, 34.94 umol, 1.5 eq) and NH₂OH·HCl (2.43 mg, 34.94 umol, 1.5 eq), then the mixture was stirred at 80° C. for 2 hr. LCMS showed the reaction was complete. The reaction mixture was concentrated in vacuum. The crude product was purified by Prep-HPLC (column: Phenomenex C18 80*30 mm*3 um; mobile phase: [water(HCl)-ACN]; B %: 15%-45%,8 min). Compound5-chloro-2-[[6-chloro-3-(4-hydroxyiminocyclohexyl)-4-quinolyl]amino]benzoic acid (2.2 mg, 4.95 umol, 21.26% yield) was obtained as a yellow solid. ¹H NMR (400 MHz, Acetonitrile-d3) δ 9.68-9.57 (m, 1H), 8.97-8.91 (s, 1H), 8.10-8.05 (m, 1H), 7.99 (d, J=2.6 Hz, 1H), 7.84-7.80 (m, 1H), 7.70-7.65 (m, 1H), 7.18 (dd, J=2.6, 9.0 Hz, 1H), 6.22-6.12 (m, 1H), 3.41-3.29 (m, 1H), 3.25-3.14 (m, 1H), 2.09-1.99 (m, 4H), 1.91-1.72 (m, 4H). MS (M+H)⁺=444.1.

### Example 141—Synthesis of 398A

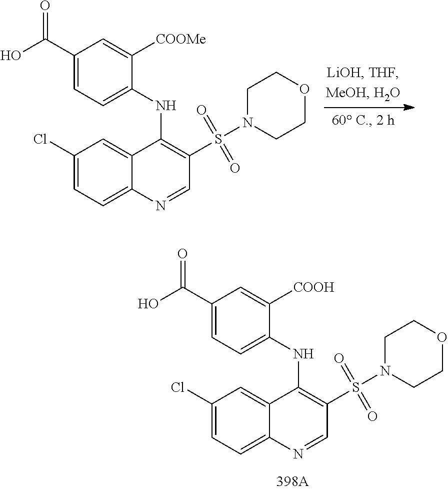 [SMILES: COC(=O)c1cc(C(=O)O)ccc1Nc1c(S(=O)(=O)N2CCOCC2)cnc2ccc(Cl)cc12 ; O=C(O)c1ccc(Nc2c(S(=O)(=O)N3CCOCC3)cnc3ccc(Cl)cc23)c(C(=O)O)c1]

### Synthesis of 4-[(6-chloro-3-morpholinosulfonyl-4-quinolyl)amino]benzene-1,3-dicarboxylic acid (398A)

To a solution of 4-[(6-chloro-3-morpholinosulfonyl-4-quinolyl)amino]-3-methoxycarbonyl-benzoic acid (20 mg, 39.53 umol, 1 eq) in THF (1 mL), MeOH (0.1 mL) and H₂O (0.1 mL) was added LiOH (1.89 mg, 79.06 umol, 2 eq), the reaction was stirred at 60° C. for 2 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water (0.04% TFA)-ACN]; B %: 25%-60%, 8 min), afford crude product 10 mg. The crude product was purified by prep-HPLC (column: PhenomenexC18 75*30 mm*3 um; mobile phase: [water(0.01% NH₃H₂O)-ACN]; B %: 5%-25%,8 min) Compound 4-[(6-chloro-3-morpholinosulfonyl-4-quinolyl)amino]benzene-1,3-dicarboxylic acid (3.3 mg, 6.64 umol, 16.79% yield, 98.93% purity) was obtained as a yellow solid. ¹H NMR (400 MHz, DMSO-d6+D₂O) δ=9.12 (s, 1H), 8.55 (d, J=2.1 Hz, 1H), 8.14 (d, J=9.0 Hz, 1H), 7.91 (dd, J=2.3, 9.1 Hz, 1H), 7.70 (dd, J=2.0, 8.6 Hz, 1H), 7.66 (d, J=2.3 Hz, 1H), 6.49 (d, J=8.6 Hz, 1H), 3.45-3.38 (m, 2H), 3.32-3.25 (m, 2H), 3.08-2.96 (m, 4H). MS (M+H)⁺=492.1.

### Example 142—Synthesis of 399A

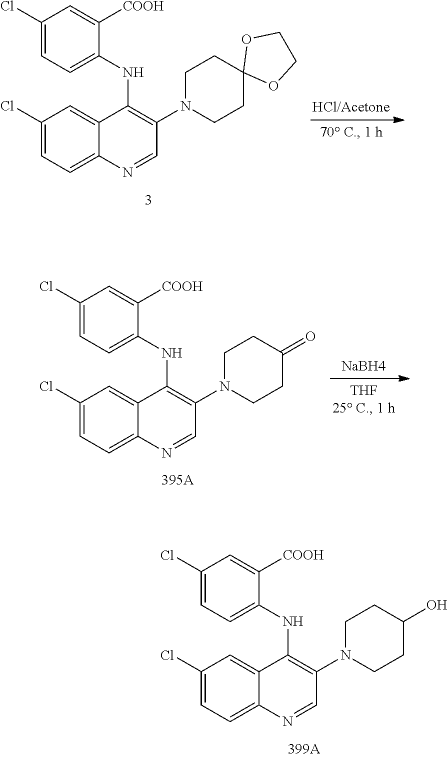

### Synthesis of 5-chloro-2-[[6-chloro-3-(4-oxo-1-piperidyl)-4-quinolyl]amino]benzoic acid (395A)

To a stirred solution of 5-chloro-2-[[6-chloro-3-(1,4-dioxa-8-azaspiro [4.5]decan-8-yl)-4-quinolyl]amino]benzoic acid (50 mg, 105.41 umol, 1 eq) in ACETONE (2 mL) was added, HCL (3 M, 0.6 mL, 17.08 eq), the reaction was stirred at 70° C. for 1 h. LCMS showed starting material was consumed completely and the MS of desired product was detected. The reaction was concentrated dry in vacuum. Compound 5-chloro-2-[[6-chloro-3-(4-oxo-1-piperidyl)-4-quinolyl]amino]benzoic acid (20 mg, 46.48 umol, 44.10% yield) was obtained as a yellow solid.

### Synthesis of 5-chloro-2-[[6-chloro-3-(4-hydroxy-1-piperidyl)-4-quinolyl]amino]benzoic acid (399A)

To a stirred solution of 5-chloro-2-[[6-chloro-3-(4-oxo-1-piperidyl)-4-quinolyl]amino]benzoic acid (20 mg, 46.48 umol, 1 eq) in THF (1 mL) was added, NaBH₄ (3.52 mg, 92.96 umol, 2 eq), the reaction was stirred at 25° C. for 1 h under N₂. LCMS showed starting material was consumed completely and the MS of desired product was detected. The reaction mixture was quenched with 2N HCl (2 ml). The solution was purified by prep-HPLC (column: Phenomenex Luna C18 150*30 mm*5 um; mobile phase: [water (TFA)-ACN]; B %: 20%-50%, 8 min). Compound 5-chloro-2-[[6-chloro-3-(4-hydroxy-1-piperidyl)-4-quinolyl]amino]benzoic acid (4 mg, 6.98 umol, 15.01% yield, 95.31% purity, TFA) was obtained as a yellow solid. ¹H NMR (400 MHz, DMSO-d₆) δ ppm 8.71 (s, 1H), 8.16 (d, J=2.00 Hz, 1H), 8.00 (d, J=9.01 Hz, 1H), 7.87 (d, J=2.50 Hz, 1H), 7.80 (dd, J=9.07, 2.19 Hz, 1H), 7.48 (dd, J=8.82, 2.56 Hz, 1H), 6.87 (d, J=8.76 Hz, 1H), 3.45 (dq, J=8.22, 4.26 Hz, 1H), 2.99-3.05 (m, 2H), 2.70 (br t, J=10.01 Hz, 2H), 1.42-1.52 (m, 2H), 0.94-1.07 (m, 2H). MS (M+H)⁺=432.0.

### Example 143—Synthesis of 400A

Synthetic scheme is provided in FIG. 39L.

### Synthesis of methyl 5-chloro-2-[[6-chloro-3-(4-oxocyclohexyl)-4-quinolyl]amino]benzoate (1)

To a solution of methyl 5-chloro-2-[[6-chloro-3-(1,4-dioxaspiro[4.5]decan-8-yl)-4-quinolyl]amino]benzoate (50 mg, 102.59 umol, 1 eq) in acetone (0.7 mL) was added HCl (3 M, 333.33 μL, 9.75 eq). The mixture was stirred at 70° C. for 1 h. LCMS showed the starting material was consumed completely and desired MS was detected. The reaction mixture was concentrated to dryness to give the crude product. Then the reaction mixture was basified by sat. NaHCO₃ to pH=8-10 at 0° C. and extracted with Ethyl acetate (3 mL*3). The combined organic layers were washed with brine (3 mL), dried over Na₂SO₄ and concentrated to dryness to give residue. Compound methyl 5-chloro-2-[[6-chloro-3-(4-oxocyclohexyl)-4-quinolyl]amino]benzoate (40 mg, crude) was obtained as a yellow oil. MS (M+H)⁺=443.10.

### Synthesis of methyl 5-chloro-2-[[6-chloro-3-(4-hydroxycyclohexyl)-4-quinolyl]amino]benzoate (2)

To a solution of methyl 5-chloro-2-[[6-chloro-3-(4-oxocyclohexyl)-4-quinolyl]amino]benzoate (35 mg, 78.95 umol, 1 eq) in MeOH (1 mL) was added NaBH₄ (8.96 mg, 236.85 umol, 3 eq) in portions at 0° C. The mixture was stirred at 20° C. for 2 h under N₂ atmosphere. LCMS showed the starting material was consumed completely and desired MS was detected. The reaction was quenched by H₂O (2 ml) at 0° C. Then the mixture was extracted with ethyl acetate (3 ml*3), the combined organic layers were dried over Na₂SO₄, filtered and concentrated under reduced pressure to give a residue. The residue was purified by prep-HPLC (Waters Xbridge Prep OBD C18 150*40 mm*10 um column; 45-65% acetonitrile in an a 10 mM ammonium bicarbonate solution in water, 8 min gradient). Compound methyl 5-chloro-2-[[6-chloro-3-(4-hydroxycyclohexyl)-4-quinolyl]amino]benzoate (27 mg, 60.63 umol, 76.79% yield) was obtained as a white solid. MS (M+H)⁺=445.15.

### Synthesis of methyl 5-chloro-2-[[6-chloro-3-(4-fluorocyclohexyl)-4-quinolyl]amino]benzoate (3)

To a solution of methyl 5-chloro-2-[[6-chloro-3-(4-hydroxycyclohexyl)-4-quinolyl]amino]benzoate (25 mg, 56.14 μmol, 1 eq) in DCM (1.5 mL) was added DAST (36.19 mg, 224.55 umol, 29.67 μL, 4 eq) in DCM (0.5 mL) at 0° C. The mixture was stirred at 20° C. for 14 h under N₂ atmosphere. LCMS showed 7% of starting material remained, 32% of desired product was detected. The reaction mixture was basified by sat. NaHCO₃ to pH=8-10 at 0° C. Then 3 mL of water was added to the reaction, the reaction mixture was extracted with dichloromethane (3 mL*3). The combined organic layers were washed with brine (3 mL) and dried over Na₂SO₄. The combined organic layer was concentrated to dryness to give a residue. Compound methyl 5-chloro-2-[[6-chloro-3-(4-fluorocyclohexyl)-4-quinolyl]amino]benzoate (19.5 mg, 43.59 umol, 77.65% yield) was obtained as a yellow oil. MS (M+H)⁺=447.10.

### Synthesis of methyl 5-chloro-2-[[6-chloro-3-(4-fluorocyclohexyl)-4-quinolyl]amino]benzoic acid (400A)

To a solution of methyl 5-chloro-2-[[6-chloro-3-(4-fluorocyclohexyl)-4-quinolyl]amino]benzoate (17 mg, 38.00 umol, 1 eq) in THF (0.9 mL) and MeOH (0.3 mL) was added LiOH·H₂O (2 M, 38.00 μL, 2 eq). The mixture was stirred at 60° C. for 1 h. LCMS showed the starting material was consumed completely and desired MS was detected. The reaction mixture was concentrated to dryness to give the crude product. Then 1 mL H₂O was added to it. Then sat.citric acid was added to the above mixture at 0° C. until PH=3˜4. The reaction mixture was extracted with Ethyl acetate (2 mL*3). The combined organic layers were washed with brine (2 mL) and dried over Na₂SO₄. The combined organic layer was concentrated to dryness to give a residue. The crude product was purified by prep-HPLC (Phenomenex Luna 80*30 mm*3 um column; 25-55% acetonitrile in an a 0.05% hydrochloric acid solution in water, 8 min gradient). Compound 5-chloro-2-[[6-chloro-3-(4-fluorocyclohexyl)-4-quinolyl]amino]benzoic acid (0.4 mg, 9.02e-1 umol, 2.37% yield, 97.763% purity) was obtained as a yellow solid. ¹H NMR (400 MHz, METHANOL-d₄) δ 8.89 (s, 1H), 8.06-8.03 (m, 2H), 7.89-7.83 (m, 2H), 7.34 (dd, J=2.4, 8.9 Hz, 1H), 6.57-6.55 (m, 1H), 4.68-4.62 (m, 1H), 3.04 (br t, J=12.3 Hz, 1H), 2.22-1.93 (m, 4H), 1.87-1.63 (m, 4H). MS (M+H)⁺=433.0.

### Example 144—Synthesis of 401A

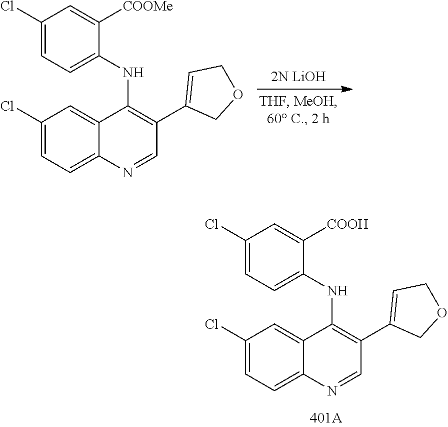

### Synthesis of 5-chloro-2-[[6-chloro-3-(2,5-dihydrofuran-3-yl)-4-quinolyl]amino]benzoic acid (401A)

To a solution of methyl 5-chloro-2-[[6-chloro-3-(2,5-dihydrofuran-3-yl)-4-quinolyl]amino]benzoate (20 mg, 48.16 umol, 1 eq) in THF (0.5 mL), MeOH (0.1 mL) and H₂O (0.1 mL) was added LiOH·H₂O (4.04 mg, 96.32 umol, 2 eq), the reaction was stirred at 60° C. for 2 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 30%-60%,8 min). Compound 5-chloro-2-[[6-chloro-3-(2,5-dihydrofuran-3-yl)-4-quinolyl]amino]benzoic acid (3.0 mg, 6.85 umol, 14.23% yield, 100% purity, HCl) was obtained as a yellow solid. ¹H NMR (400 MHz, DMSO-d₆+D₂O) δ=8.82 (s, 1H), 8.35 (d, J=1.4 Hz, 1H), 8.05 (d, J=9.0 Hz, 1H), 7.92 (dd, J=1.9, 9.0 Hz, 1H), 7.85 (d, J=2.5 Hz, 1H), 7.44 (dd, J=2.5, 8.8 Hz, 1H), 6.77 (d, J=8.8 Hz, 1H), 6.10 (br s, 1H), 4.55 (br s, 2H), 4.35 (br s, 2H). MS (M+H)⁺=400.9.

### Example 145—Synthesis of 402A

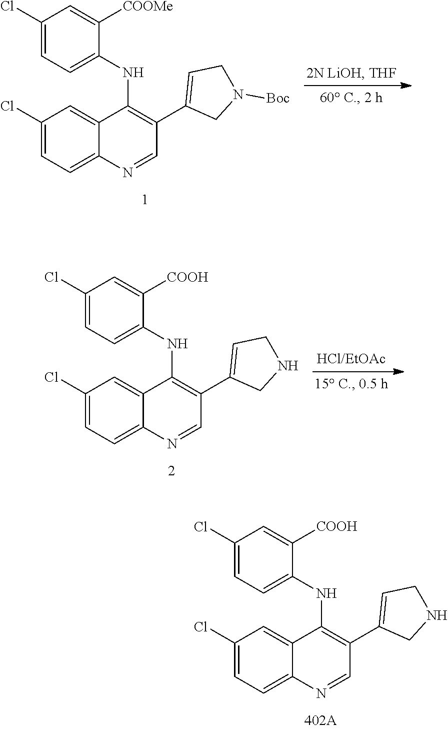

### Synthesis of 2-[[3-(1-tert-butoxycarbonyl-2,5-dihydropyrrol-3-yl)-6-chloro-4-quinolyl]amino]-5-chloro-benzoic acid (2)

To a solution of tert-butyl 3-[6-chloro-4-(4-chloro-2-methoxycarbonyl-anilino)-3-quinolyl]-2,5-dihydropyrrole-1-carboxylate (80 mg, 155.52 umol, 1 eq) in THF (0.5 mL), MeOH (0.1 mL) and H₂O (0.1 mL) was added LiOH·H₂O (13.05 mg, 311.04 umol, 2 eq), the reaction was stirred at 60° C. for 2 h. LCMS showed starting material was consumed completely and the MS of desired product was detected. The reaction mixture was concentrated under reduced pressure to give a residue. Compound 2-[[3-(1-tert-butoxycarbonyl-2,5-dihydropyrrol-3-yl)-6-chloro-4-quinolyl]amino]-5-chloro-benzoic acid (70 mg, 139.90 umol, 89.95% yield) was obtained as a yellow solid.

### Synthesis of 5-chloro-2-((6-chloro-3-(2,5-dihydro-1H-pyrrol-3-yl)quinolin-4-yl)amino)benzoic acid (402A)

A solution of 2-[[3-(1-tert-butoxycarbonyl-2,5-dihydropyrrol-3-yl)-6-chloro-4-quinolyl]amino]-5-chloro-benzoic acid (70 mg, 139.90 umol, 1 eq) in HCl/EtOAc (1 mL), the reaction was stirred at 15° C. for 1 h. LCMS showed starting material was consumed completely and the MS of desired product was detected. The reaction mixture was concentrated under reduced pressure to give a residue. The residue was purified by prep-HPLC (column: Phenomenex Luna 80*30 mm*3 um; mobile phase: [water(0.04% HCl)-ACN]; B %: 15%-35%,8 min) Compound 5-chloro-2-[[6-chloro-3-(2,5-dihydro-1H-pyrrol-3-yl)-4-quinolyl]amino]benzoic acid (6.4 mg, 14.65 umol, 10.48% yield, 100% purity, HCl) was obtained as a yellow solid. ¹H NMR (400 MHz, DMSO-d₆+D₂O) δ=8.98 (s, 1H), 8.19 (d, J=1.6 Hz, 1H), 8.15-8.09 (m, 1H), 7.96-7.88 (m, 2H), 7.41 (dd, J=2.6, 8.8 Hz, 1H), 6.59 (d, J=8.9 Hz, 1H), 6.26 (br s, 1H), 4.22-4.05 (m, 2H), 3.98-3.82 (m, 2H). MS (M+H)⁺=400.0.

### Example 146—Synthesis of 403A

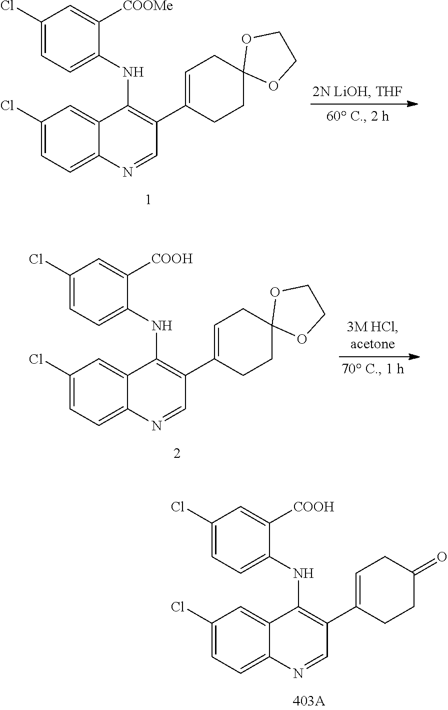

### Synthesis of 5-chloro-2-[[6-chloro-3-(1,4-dioxaspiro[4.5]dec-7-en-8-yl)-4-quinolyl]amino]benzoic acid (2)

To a solution of methyl 5-chloro-2-[[6-chloro-3-(1,4-dioxaspiro[4.5]dec-7-en-8-yl)-4-quinolyl]amino]benzoate (120 mg, 247.24 umol, 1 eq) in THF (1 mL), MeOH (0.2 mL) and H₂O (0.2 mL) was added LiOH·H₂O (20.75 mg, 494.48 umol, 2 eq), the reaction was stirred at 60° C. for 1 h. LCMS showed starting material was consumed completely and the MS of desired product was detected. The reaction mixture was concentrated in vacuum. Compound 5-chloro-2-[[6-chloro-3-(1,4-dioxaspiro[4.5]dec-7-en-8-yl)-4-quinolyl]amino]benzoic acid (80 mg, 169.73 umol, 68.65% yield) was obtained as a yellow solid. MS (M+H)⁺=471.2.

### Synthesis of 5-chloro-2-[[6-chloro-3-(4-oxocyclohexen-1-yl)-4-quinolyl]amino]benzoic acid (403A)

To a solution of 5-chloro-2-[[6-chloro-3-(1,4-dioxaspiro[4.5]dec-7-en-8-yl)-4-quinolyl]amino]benzoic acid (80 mg, 169.73 umol, 1 eq) in ACETONE (2 mL) was added HCl (3 M, 568.14 uL, 10.04 eq), the reaction was stirred at 70° C. for 1 h. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 10%-40%,8 min), afford crude product 20 mg. The crude product was purified by prep-HPLC (column: Waters Xbridge BEH C18 100*30 mm*10 um; mobile phase: [water(10 mmol NH₄HCO₃)-ACN]; B %: 25%-45%,8 min) Compound 5-chloro-2-[[6-chloro-3-(4-oxocyclohexen-1-yl)-4-quinolyl]amino]benzoic acid (0.3 mg, 6.58e-1 umol, 3.88e-1% yield, 93.77% purity) was obtained as a yellow solid. ¹H NMR (400 MHz, DMSO-d₆) δ=8.80 (s, 1H), 8.08 (d, J=9.0 Hz, 1H), 7.98 (d, J=2.1 Hz, 1H), 7.87 (d, J=2.5 Hz, 1H), 7.79 (dd, J=2.2, 8.9 Hz, 2H), 7.28-7.23 (m, 1H), 6.40 (d, J=8.9 Hz, 1H), 6.08-6.00 (m, 1H), 2.94 (br d, J=2.0 Hz, 2H), 2.55 (br s, 2H), 2.21 (br dd, J=2.9, 5.8 Hz, 2H). MS (M+H)⁺=427.1.

### Example 147—Synthesis of 404A

Synthetic scheme is provided in FIG. 39M.

### Synthesis of methyl 2-[(3-bromo-6-chloro-4-quinolyl)amino]-5-chloro-benzoate (2)

To a solution of 3-bromo-4,6-dichloro-quinoline (8.4 g, 30.33 mmol, 1 eq) in acetonitrile (100 mL) was added methyl 2-amino-5-chloro-benzoate (11.26 g, 60.66 mmol, 2 eq) and HCl (12 M, 505.52 uL, 0.2 eq). The mixture was stirred at 80° C. for 14 h. LCMS showed 22% of starting material remained, 65% of desired product was detected. The reaction mixture was filtered and the filter cake was concentrated in vacuo. The crude product was purified by flash column (ISCO 20 g silica, 0-100% ethyl acetate in petroleum ether, 0-17% methanol in dichloromethane, gradient over 20 min). Compound methyl 2-[(3-bromo-6-chloro-4-quinolyl)amino]-5-chloro-benzoate (2 g, 4.69 mmol, 15.48% yield) was obtained as a pale yellow solid. MS (M+H)⁺=424.80.

### Synthesis of methyl (1,3-dioxoisoindolin-2-yl) 1,4-dioxaspiro[4.5]decane-8-carboxylate (4)

To a solution of 1,4-dioxaspiro[4.5]decane-8-carboxylic acid (3.7 g, 19.87 mmol, 1 eq) in DCM (40 mL) was added 2-hydroxyisoindoline-1,3-dione (3.24 g, 19.87 mmol, 1 eq), EDCI (4.57 g, 23.84 mmol, 1.2 eq) and DMAP (728.27 mg, 5.96 mmol, 0.3 eq), the mixture was stirred at 25° C. for 1 h. LCMS showed the reaction was complete. 30 mL of Water was added to the reaction, the reaction mixture was extracted with DCM (30 mL*2). The combined organic layers were washed with brine (30 mL), dried over Na₂SO₄ and filtered. The filtrate was concentrated to dryness to give residue. The residue was purified by flash silica gel chromatography (ISCO; 40 g SepaFlash Silica Flash Column, Eluent of 5˜30% Ethyl acetate/Petroleum ether gradient at 100 m/min). (1,3-dioxoisoindolin-2-yl) 1,4-dioxaspiro[4.5]decane-8-carboxylate (5.7 g, 17.20 mmol, 86.58% yield) was obtained as a white solid. MS (M+H)⁺=332.10.

### Synthesis of methyl 5-chloro-2-[[6-chloro-3-(1,4-dioxaspiro[4.5]decan-8-yl)-4-quinolyl]amino]benzoate (5)

To a solution of (1,3-dioxoisoindolin-2-yl) 1,4-dioxaspiro[4.5]decane-8-carboxylate (388.79 mg, 1.17 mmol, 1 eq), methyl 2-[(3-bromo-6-chloro-4-quinolyl)amino]-5-chloro-benzoate (500 mg, 1.17 mmol, 1 eq), Zn (153.46 mg, 2.35 mmol, 2 eq) in DMA (2 mL) was added Ni(dtbbpy)Br₂ (114.25 mg, 234.69 umol, 0.2 eq). The mixture was stirred at 40° C. for 12 h under N₂ atmosphere. LCMS showed the starting material was consumed completely and desired MS was detected. 3 mL of water was added to the reaction, the reaction mixture was extracted with Ethyl acetate (5 mL*3). The combined organic layers were washed with brine (3 mL) and dried over Na₂SO₄. The combined organic layer was concentrated to dryness to give residue. The crude product was purified by flash column (ISCO 20 g silica, 0-40% ethyl acetate in petroleum ether, gradient over 20 min). Compound methyl 5-chloro-2-[[6-chloro-3-(1,4-dioxaspiro[4.5]decan-8-yl)-4-quinolyl]amino]benzoate (60 mg, 123.11 umol, 10.49% yield) was obtained as a yellow solid. MS (M+H)⁺=487.10.

### Synthesis of methyl 5-chloro-2-[[6-chloro-3-(1,4-dioxaspiro[4.5]decan-8-yl)-4-quinolyl]amino]benzoic acid (6)

To a solution of methyl 5-chloro-2-[[6-chloro-3-(1,4-dioxaspiro[4.5]decan-8-yl)-4-quinolyl]amino]benzoate (110 mg, 225.70 umol, 1 eq) in THF (1.2 mL) and MeOH (0.4 mL) was added LiOH·H₂O (2 M, 225.70 uL, 2 eq). The mixture was stirred at 60° C. for 6 h. LCMS showed the starting material was consumed completely and desired MS was detected. The reaction mixture was acidize with 2M HCl to adjust pH=5˜6. Then the mixture extracted with Ethyl acetate (10 mL*3). The combined organic layers were washed with brine (3 mL), dried over Na₂SO₄ and concentrated to dryness to give a residue. Compound 5-chloro-2-[[6-chloro-3-(1,4-dioxaspiro[4.5]decan-8-yl)-4-quinolyl]amino]benzoic acid (110 mg crude) was obtained as a yellow oil. MS (M+H)=473.10.

### Synthesis of methyl 5-chloro-2-[[6-chloro-3-(4-oxocyclohexyl)-4-quinolyl]amino]benzoic acid (404A)

To a solution of 5-chloro-2-[[6-chloro-3-(1,4-dioxaspiro[4.5]decan-8-yl)-4-quinolyl]amino]benzoic acid (110 mg, 232.39 umol, 1 eq) in acetone (1.5 mL) was added HCl (3 M, 0.4 mL, 5.16 eq). The mixture was stirred at 70° C. for 1 h. LCMS showed the starting material was consumed completely and desired MS was detected. The reaction mixture was concentrated to dryness to give the crude product. The crude product was purified by prep-HPLC (Phenomenex Luna 80*30 mm*3 um column; 30-50% acetonitrile in an a 0.05% hydrochloric acid solution in water, 8 min gradient). Compound 5-chloro-2-[[6-chloro-3-(4-oxocyclohexyl)-4-quinolyl]amino]benzoic acid (24.20 mg, 56.37 umol, 24.26% yield) was obtained as a yellow solid. ¹H NMR (400 MHz, DMSO-d₆) δ 9.95-9.84 (m, 1H), 9.06 (s, 1H), 8.12-8.09 (m. 1H), 7.92 (d, J=2.6 Hz, 1H), 7.86-7.74 (m, 2H), 7.42-7.32 (m, 1H), 6.50-6.31 (m, 1H), 3.19-3.06 (m, 1H), 2.58-2.53 (m, 1H), 2.45-2.36 (m, 1H), 2.31-2.19 (m, 3H), 2.15-1.97 (m, 3H). MS (M+H)+=429.0.

### Example 148—Synthesis of 404A

Synthetic scheme is shown in FIG. 39N.

### Synthesis of methyl 5-chloro-2-[[6-chloro-3-(1,4-dioxaspiro[4.5]dec-7-en-8-yl)-4-quinolyl]amino]benzoate (2)

To a solution of methyl 2-[(3-bromo-6-chloro-4-quinolyl)amino]-5-chloro-benzoate (2 g, 4.69 mmol, 1 eq) DMF (15 mL) and H₂O (3 mL) was added K₃PO₄ (2.99 g, 14.08 mmol, 3 eq), 2-(1,4-dioxaspiro[4.5]dec-7-en-8-yl)-4,4,5,5-tetramethyl-1,3,2-dioxaborolane (1.25 g, 4.69 mmol, 1 eq) and Pd(dppf)Cl₂ (343.45 mg, 469.38 umol, 0.1 eq), the mixture was purged with N₂, the reaction was stirred at 100° C. for 3 h under N₂. LCMS showed starting material was consumed completely and the MS of desired product was detected. The reaction was cooled to ambient temperature, quenched with water (70 ml) and extracted with ethyl acetate (70 ml). The organic layer was washed with water, brine, dried over anhydrous sodium sulfate, filtered and concentrated in vacuum. The residue was purified by flash column (ISCO 40 g silica, 40-60% ethyl acetate in petroleum ether, gradient over 20 min). TLC (Petroleum ether/Ethyl acetate=0:1, R_{f}=0.55) Compound methyl 5-chloro-2-[[6-chloro-3-(1,4-dioxaspiro[4.5]dec-7-en-8-yl)-4-quinolyl]amino]benzoate (800 mg, 1.65 mmol,35.12% yield) was obtained as a white solid. ¹H NMR (400 MHz, DMSO-d₆) δ=9.47-9.26 (m, 1H), 8.73-8.70 (m, 1H), 8.05-8.02 (m, 1H), 7.91-7.88 (m, 1H), 7.77-7.74 (m, 1H), 7.37-7.27 (m, 1H), 6.47-6.36 (m, 1H), 5.61-5.58 (m, 1H), 4.01-3.97 (m, 4H), 3.89-3.86 (m, 3H), 2.38-2.36 (m, 2H), 1.86-1.77 (m, 4H). MS (M+H)⁺=485.1.

### Synthesis of methyl 5-chloro-2-[[6-chloro-3-(1,4-dioxaspiro[4.5]decan-8-yl)-4-quinolyl]amino]benzoate (3)

To a solution of methyl 5-chloro-2-[[6-chloro-3-(1,4-dioxaspiro[4.5]dec-7-en-8-yl)-4-quinolyl]amino]benzoate (200 mg, 412.07 umol, 1 eq) in EtOAc (4 mL) was added PtO₂ (9.36 mg, 41.21 umol, 0.1 eq) and AcOH (2.47 mg, 41.21 umol, 2.36 uL, 0.1 eq), the mixture was stirred at 15° C. for 5 under H₂. LCMS showed starting material was consumed completely and the MS of desired product was detected. The reaction mixture was filtered, filtrate was concentrated in vacuum. The crude product was purified by prep-HPLC (column: Phenomenex Luna 80*30 mm*3 um; mobile phase: [water(0.04% HCl)-ACN]; B %: 30%-60%,8 min) Compound methyl 5-chloro-2-[[6-chloro-3-(1,4-dioxaspiro[4.5]decan-8-yl)-4-quinolyl]amino]benzoate (12 mg, 22.91 umol, 5.56% yield, HCl) was obtained as a yellow solid. MS (M+H)⁺=487.2.

### Synthesis of 5-chloro-2-[[6-chloro-3-(1,4-dioxaspiro[4.5]decan-8-yl)-4-quinolyl]amino]benzoic acid (4)

To a solution of methyl 5-chloro-2-[[6-chloro-3-(1,4-dioxaspiro[4.5]decan-8-yl)-4-quinolyl]amino]benzoate (11 mg, 22.57 umol, 1 eq) in THF (0.3 mL). MeOH (0.1 mL) and H₂O (0.1 mL) was added LiOH·H₂O (1.89 mg, 45.14 umol, 2 eq), the reaction was stirred at 60° C. for 1 h. LCMS showed starting material was consumed completely and the MS of desired product was detected. The reaction mixture was concentrated in vacuum. Compound 5-chloro-2-[[6-chloro-3-(1,4-dioxaspiro[4.5]decan-8-yl)-4-quinolyl]amino]benzoic acid (10 mg, 21.13 umol, 93.60% yield) was obtained as a yellow solid. MS (M+H)⁺=473.2.

### Synthesis of 5-chloro-2-[[6-chloro-3-(4-oxocyclohexyl)-4-quinolyl]amino]benzoic acid (404A)

To a solution of 5-chloro-2-[[6-chloro-3-(1,4-dioxaspiro[4.5]decan-8-yl)-4-quinolyl]amino]benzoic acid (10 mg, 21.13 umol, 1 eq) in ACETONE (0.5 mL) was added HCl (3 M, 0.1 mL, 14.20 eq), the reaction was stirred at 70° C. for 1 h under N₂. LCMS showed starting material was consumed completely and the MS of desired product was detected. The reaction mixture was concentrated in vacuum. The crude product was purified by prep-HPLC (column: Phenomenex Luna 80*30 mm*3 um; mobile phase: [water(0.04% HCl)-ACN]; B %: 20%-50%,8 min) Compound 5-chloro-2-[[6-chloro-3-(4-oxocyclohexyl)-4-quinolyl]amino]benzoic acid (0.6 mg, 1.29 umol, 6.10% yield, 100% purity, HCl) was obtained as a yellow solid. ¹H NMR (400 MHz, DMSO-d₆) δ=10.06 (br s, 1H), 9.02 (s, 1H), 8.17 (d, J=9.0 Hz, 1H), 7.94 (d, J=2.6 Hz, 1H), 7.92-7.87 (m, 1H), 7.86 (d, J=2.0 Hz, 1H), 7.46 (dd, J=2.4, 8.8 Hz, 1H), 6.69 (br d, J=7.0 Hz, 1H), 3.43-3.34 (m, 1H), 2.54 (s, 1H), 2.47-2.35 (m, 1H), 2.31-2.17 (m, 3H), 2.15-1.91 (m, 3H). MS (M+H)⁺=429.0.

### Example 149—Synthesis of 411A

Synthetic scheme is provided in FIG. 39O.

### Synthesis of 5-[(4-chloro-3-methyl-anilino)methylene]-2,2-dimethyl-1,3-dioxane-4,6-dione (2)

To a solution of 5-(methoxymethylene)-2,2-dimethyl-1,3-dioxane-4,6-dione (6.57 g, 35.31 mmol, 1 eq) in i-PrOH (100 mL) was added 4-chloro-3-methyl-aniline (5 g, 35.31 mmol, 1 eq) in portions at 50° C. The mixture was stirred at 80° C. for 2.5 h. LCMS showed the starting material was consumed completely and desired MS was detected. The reaction mixture was filtered and the filter cake was concentrated in vacuo. Compound 5-[(4-chloro-3-methyl-anilino)methylene]-2,2-dimethyl-1,3-dioxane-4,6-dione (9 g, 30.43 mmol, 86.19% yield) was obtained as a pale yellow solid. ¹H NMR (400 MHz, DMSO-d6) δ 11.21 (br s, 1H), 8.56 (br s, 1H), 7.62 (s, 1H), 7.50-7.37 (m, 2H), 2.34 (s, 3H), 1.67 (s, 6H). MS (M+H)⁺=296.1.

### Synthesis of 6-chloro-5-methyl-quinolin-4-ol (3) and 6-chloro-7-methyl-quinolin-4-ol (3A)

A mixture of 5-[(4-chloro-3-methyl-anilino)methylene]-2,2-dimethyl-1,3-dioxane-4,6-dione (660 mg, 2.23 mmol, 1 eq) in diphenyl ether (15 mL) was stirred at 250° C. for 1 h. LCMS showed the starting material was consumed completely and desired MS was detected. The mixture was allowed to 20° C. then poured into hexanes (30 mL). The resulting solid was collected by filtration and washed with hexanes. The residue was purified by prep-HPLC (Waters Xbridge BEH C18 250*50 mm*10 um column; 15-35% acetonitrile in an a 10 mM ammonium bicarbonate solution in water, 10 min gradient). Compound 6-chloro-7-methyl-quinolin-4-ol (225 mg, 1.16 mmol, 52.06% yield) was obtained as a white solid. Compound 6-chloro-5-methyl-quinolin-4-ol (350 mg, 1.81 mmol, 80.99% yield) was obtained as a white solid. ¹H NMR (400 MHz, METHANOL-d4) δ 8.18 (s, 1H), 7.94 (d, J=7.4 Hz, 1H), 7.49 (s, 1H), 6.29 (d, J=7.3 Hz, 1H), 2.51 (s, 3H). MS (M+H)⁺=194.1. ¹H NMR (400 MHz, METHANOL-d₄) δ 7.79 (d, J=7.3 Hz, 1H), 7.62 (d, J=9.0 Hz, 1H), 7.34 (d, J=8.9 Hz, 1H), 6.23 (d, J=7.3 Hz, 1H), 2.99 (s, 3H).

### Synthesis of 6-chloro-4-hydroxy-7-methyl-quinoline-3-sulfonyl chloride (4A)

A mixture of 6-chloro-7-methyl-quinolin-4-ol (400 mg, 2.07 mmol, 1 eq) and HSO₃Cl (4 mL) was stirred at 100° C. for 12 h. LCMS showed the starting material was consumed completely and desired MS was detected. The reaction mixture was poured into ice water and filtered. The filter cake was concentrated in vacuo. Compound 6-chloro-4-hydroxy-7-methyl-quinoline-3-sulfonyl chloride (610 mg, crude) was obtained as a brown solid. MS (M+H)⁺=292.0.

### Synthesis of 6-chloro-7-methyl-3-thiomorpholinosulfonyl-quinolin-4-ol (5A)

To a solution of 6-chloro-4-hydroxy-7-methyl-quinoline-3-sulfonyl chloride (600 mg, 2.05 mmol, 1 eq) in DCM (8 mL) was added Et₃N (623.47 mg, 6.16 mmol, 857.60 uL, 3 eq) and thiomorpholine (423.85 mg, 4.11 mmol, 388.85 uL, 2 eq). The mixture was stirred at 25° C. for 12 h. LCMS showed the starting material was consumed completely and desired MS was detected. The reaction mixture was filtered and the filter cake was concentrated in vacuo. Compound 6-chloro-7-methyl-3-thiomorpholinosulfonyl-quinolin-4-ol (610 mg, crude) was obtained as a white solid. MS (M+H)⁺=359.1.

### Synthesis of 4-[(4,6-dichloro-7-methyl-3-quinolyl)sulfonyl]thiomorpholine (6A)

A mixture of 6-chloro-7-methyl-3-thiomorpholinosulfonyl-quinolin-4-ol (600 mg, 1.67 mmol, 1 eq) in POCl₃ (6 mL) was stirred at 110° C. for 2 h. LCMS showed the starting material was consumed completely and desired MS was detected. The reaction mixture was concentrated to dryness to give the crude product. Then Ethyl acetate (10 mL) was added to it and poured into ice water (10 mL), basified by sat. NaHCO₃ to pH=8-9 at 0° C. The reaction mixture was extracted with Ethyl acetate (10 mL×3). The combined organic layers were washed with brine (10 mL), dried over Na₂SO₄ and concentrated to dryness to give residue. The crude product was purified by flash column (ISCO 20 g silica, 0-56% ethyl acetate in petroleum ether, gradient over 20 min). Compound 4-[(4,6-dichloro-7-methyl-3-quinolyl)sulfonyl]thiomorpholine (100 mg, crude) was obtained as a white solid. MS (M+H)⁺=377.0.

### Synthesis of 5-chloro-2-[(6-chloro-7-methyl-3-thiomorpholinosulfonyl-4-quinolyl)amino]benzoic acid (411A)

To a solution of 4-[(4,6-dichloro-7-methyl-3-quinolyl)sulfonyl]thiomorpholine (100 mg, 265.04 umol, 1 eq) in EtOH (3 mL) and CHCl₃ (0.6 mL) was added 2-amino-5-chloro-benzoic acid (90.95 mg, 530.07 umol, 2 eq). The mixture was stirred at 80° C. for 2 h. LCMS showed the starting material was consumed completely and desired MS was detected. The reaction mixture was concentrated to dryness to give the crude product. The crude product was purified by prep-HPLC (Phenomenex luna C18 80*40 mm*3 um column; 40-75% acetonitrile in an a 0.05% hydrochloric acid solution in water, 7 min gradient). Compound 5-chloro-2-[(6-chloro-7-methyl-3-thiomorpholinosulfonyl-4-quinolyl)amino]benzoic acid (33.18 mg, 64.75 umol, 24.43% yield) was obtained as a yellow solid. ¹H NMR (400 MHz, METHANOL-d₄) δ 9.15 (s, 1H), 8.13 (d, J=2.5 Hz, 1H), 7.99 (s, 1H), 7.66 (s, 1H), 7.44 (dd, J=2.4, 8.8 Hz, 1H), 6.92 (d, J=8.8 Hz, 1H), 3.51 (t, J=5.0 Hz, 4H), 2.66-2.54 (m, 7H). MS (M+H)⁺=512.0.

### Example 150—Synthesis of 413A

Synthetic scheme is provided in FIG. 39P.

### 5-[[4-chloro-3-(trifluoromethyl)anilino]methylene]-2,2-dimethyl-1,3-dioxane-4,6-dione (2)

To a mixture of 5-(methoxymethylene)-2,2-dimethyl-1,3-dioxane-4,6-dione (4.76 g, 25.57 mmol, 1 eq) in i-PrOH (60 mL) added 4-chloro-3-(trifluoromethyl)aniline (5.00 g, 25.57 mmol, 3.60 mL, 1 eq) at 50° C., then the mixture was stirred at 80° C. for 3 h. LC-MS showed the starting material was consumed completely and desired product was detected. The reaction mixture was filtered and the filter cake was concentrated in vacuo. Compound 5-[[4-chloro-3-(trifluoromethyl)anilino]methylene]-2,2-dimethyl-1,3-dioxane-4,6-dione (8.1 g, 23.16 mmol, 90.60% yield) was obtained as a pale yellow solid. MS (M+H)⁺=350.2.

### 6-chloro-7-(trifluoromethyl)quinolin-4-ol (3A)

A mixture of 5-[[4-chloro-3-(trifluoromethyl)anilino]methylene]-2,2-dimethyl-1,3-dioxane-4,6-dione (700 mg, 2.00 mmol, 1 eq) in diphenyl ether (20 mL) was stirred at 250° C. for 1 h. LC-MS showed the starting material was consumed completely and desired product was detected. The mixture was allowed to 20° C. then poured into hexanes (10 mL). The resulting solid was collected by filtration. Compound 6-chloro-7-(trifluoromethyl)quinolin-4-ol (3 g, 10.81 mmol, 60.02% yield) was obtained as a brown solid. MS (M+H)⁺=248.2.

### 6-chloro-4-hydroxy-7-(trifluoromethyl)quinoline-3-sulfonyl chloride (4A)

A mixture of 6-chloro-7-(trifluoromethyl)quinolin-4-ol (1 g, 4.04 mmol, 1 eq) in HSO₃Cl (10 mL) was stirred at 100° C. for 2 h. LC-MS showed the starting material was consumed completely and desired product was detected. The reaction mixture was filtered and the filter cake was concentrated in vacuo. Compound 6-chloro-4-hydroxy-7-(trifluoromethyl)quinoline-3-sulfonyl chloride (1.17 g, 884.46 umol, 21.90% yield) was obtained as a pale yellow solid. MS (M+H)⁺=346.1.

### 6-chloro-3-thiomorpholinosulfonyl-7-(trifluoromethyl)quinolin-4-ol (5A)

155. To a solution of 6-chloro-4-hydroxy-7-(trifluoromethyl)quinoline-3-sulfonyl chloride (1.1 g, 3.18 mmol, 1 eq) in DCM (15 mL) was added TEA (964.80 mg, 9.53 mmol, 1.33 mL, 3 eq) and thiomorpholine (655.89 mg, 6.36 mmol, 601.73 uL, 2 eq) at 0° C. The mixture was stirred at 25° C. for 2 h. LC-MS showed the starting material was consumed completely and desired product was detected. The reaction mixture was filtered and the filter cake was concentrated in vacuo. The crude product was purified by prep-HPLC (Waters Xbridge BEH C18 250*50 mm*10 um column; 30-50% acetonitrile in an a 10 mM ammonium bicarbonate solution in water, 11 min gradient). Compound 6-chloro-3-thiomorpholinosulfonyl-7-(trifluoromethyl)quinolin-4-ol (130 mg, 314.90 umol, 9.91% yield) was obtained as a pale yellow solid. MS (M+H)⁺=413.1.

### 4-[[4,6-dichloro-7-(trifluoromethyl)-3-quinolyl]sulfonyl]thiomorpholine (6A)

A mixture of 6-chloro-3-thiomorpholinosulfonyl-7-(trifluoromethyl)quinolin-4-ol (120 mg, 290.67 umol, 1 eq) in POCl₃ (3 mL) was stirred at 120° C. for 4 hr under N₂ atmosphere. LC-MS showed the starting material was consumed completely and desired product was detected. The reaction mixture was concentrated to dryness to give the crude product. Then Ethyl acetate (5 mL) was added to it and then poured into ice water (5 mL). The reaction mixture was filtered and the filter cake was concentrated in vacuo. Compound 4-[[4,6-dichloro-7-(trifluoromethyl)-3-quinolyl]sulfonyl]thiomorpholine (120 mg, 266.08 umol, 91.54% yield) was obtained as a pale yellow solid. MS (M+H)¹=431.0.

### 5-chloro-2-[[6-chloro-3-thiomorpholinosulfonyl-7-(trifluoromethyl)-4-quinolyl]amino]benzoic acid (413A)

To a solution of 4-[[4,6-dichloro-7-(trifluoromethyl)-3-quinolyl]sulfonyl]thiomorpholine (110 mg, 255.05 umol, 1 eq) in EtOH (2 mL) and CH₃Cl (0.4 mL) was added 2-amino-5-chloro-benzoic acid (43.76 mg, 255.05 umol, 1 eq). The mixture was stirred at 80° C. for 2 h. LC-MS showed the starting material was consumed completely and desired product was detected. 5 mL of water was added to the reaction, the reaction mixture was extracted with Ethyl acetate (5 mL×2). The combined organic layers were washed with brine (5 mL), dried over Na₂SO₄ and concentrated to dryness to give residue. The crude product was purified by prep-HPLC (Phenomenex Luna 80*30 mm*3 um column; 55-90% acetonitrile in an a 0.05% hydrochloricacid solution in water, 8 min gradient). Compound 5-chloro-2-[[6-chloro-3-thiomorpholinosulfonyl-7-(trifluoromethyl)-4-quinolyl]amino]benzoic acid (12.90 mg, 21.76 umol, 8.53% yield) was obtained as a yellow solid. ¹H NMR (400 MHz, METHANOL-d4) δ 9.27 (s, 1H), 8.48 (s, 1H), 8.12-8.10 (d, J=2.4, 1H), 7.86 (s, 1H), 7.43-7.38 (m, 1H), 6.82-6.77 (d, J=8.8, 1H), 3.51-3.45 (m, 4H), 2.62-2.50 (m, 4H). MS (M+H)⁺=566.0.

### Example 151—Synthesis of 414A

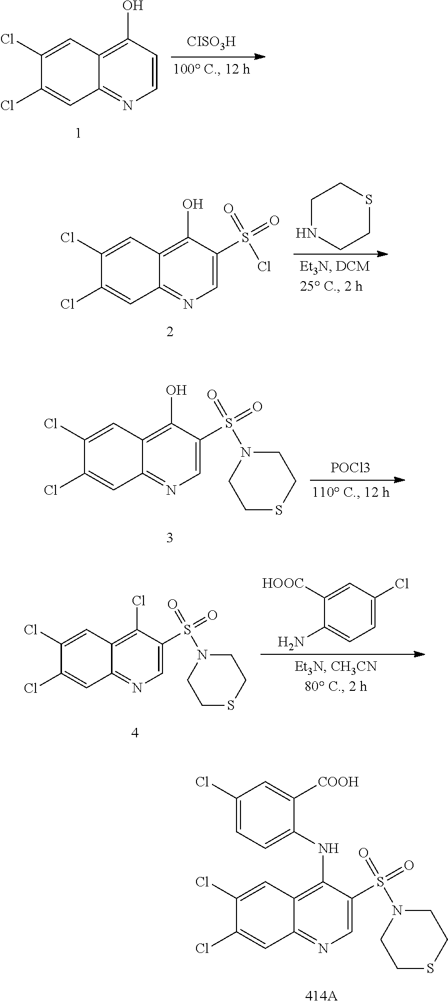

### Synthesis of 6,7-dichloro-4-hydroxy-quinoline-3-sulfonyl chloride (2)

A mixture of 6,7-dichloroquinolin-4-ol (200 mg, 934.37 umol, 1 eq) and HSO₃Cl (2 mL) were stirred at 100° C. for 12 h. The mixture was poured into 5 mL ice water and the solid formed. The mixture was filtered and the filter cake was collected by filtration and concentrated in vauo. 6,7-dichloro-4-hydroxy-quinoline-3-sulfonyl chloride (270 mg, crude) was obtained as a brown solid. MS (M+H)⁺=311.9.

### Synthesis of 6,7-dichloro-3-thiomorpholinosulfonyl-quinolin-4-ol (3)

164. To a solution of 6,7-dichloro-4-hydroxy-quinoline-3-sulfonyl chloride (260 mg, 831.85 umol, 1 eq) in DCM (3 mL) were added Et₃N (252.52 mg, 2.50 mmol, 347.35 uL, 3 eq) and thiomorpholine (171.67 mg, 1.66 mmol, 157.50 uL, 2 eq), the mixture was stirred at 25° C. for 2 h. LCMS showed the starting material was consumed completely and the desired MS was detected. The mixture was filtered and the filter cake was collected and concentrated in vacuo. 6,7-dichloro-3-thiomorpholinosulfonyl-quinolin-4-ol (220 mg, crude) was obtained as a gray solid. MS (M+H)⁺=378.9.

### Synthesis of 4-[(4,6,7-trichloro-3-quinolyl)sulfonyl]thiomorpholine (4)

165. A mixture of 6,7-dichloro-3-thiomorpholinosulfonyl-quinolin-4-ol (100 mg, 263.66 umol, 1 eq) in POCl₃ (1 mL) were stirred at 110° C. for 12 h. LCMS showed desired MS was detected and the reaction was complete. The mixture was concentrated in vacuo. 4-[(4,6,7-trichloro-3-quinolyl)sulfonyl]thiomorpholine (130 mg, crude) was obtained as pale solid. MS (M+H)⁺=397.0.

### Synthesis of 5-chloro-2-[[6-chloro-3-(4,4-dichloro-1-piperidyl)-4-quinolyl]amino]benzoic acid (414A)

To a solution of 4-[(4,6,7-trichloro-3-quinolyl)sulfonyl]thiomorpholine (100 mg, 251.43 umol, 1 eq) in CH₃CN (1 mL) were added 2-amino-5-chloro-benzoic acid (51.77 mg, 301.71 umol, 1.2 eq) and TEA (76.33 mg, 754.29 umol, 104.99 uL, 3 eq), the mixture was stirred at 80° C. for 2 h. LCMS showed the starting material was consumed completely and the desired MS was detected. The mixture was concentrated under reduced pressure to give the crude product. The crude product was purified by prep-HPLC (Phenomenex Luna 80*30 mm*3 um column; 25-75% acetonitrile in an a 0.05% hydrochloric acid solution in water, 8 min gradient). 5-chloro-2-[(6,7-dichloro-3-thiomorpholinosulfonyl-4-quinolyl)amino]benzoic acid (3.8 mg, 6.72 umol, 2.67% yield, 94.18% purity) was obtained as a yellow solid. ¹H NMR (400 MHz, METHANOL-d4) δ=9.21 (s, 1H), 8.25 (s, 1H), 8.14 (d, J=2.5 Hz, 1H), 7.82 (s, 1H), 7.46-7.43 (dd, J=2.8 Hz, 8.8 Hz, 1H), 6.94-6.91 (d, J=8.8 Hz, 1H), 3.54-3.48 (m, 4H), 2.68-2.57 (m, 4H). MS (M+H)⁺=532.0.

### Example 152—Synthesis of 415A

Synthetic scheme is provided in FIG. 39Q.

### 5-[(4-chloro-3-fluoro-anilino)methylene]-2,2-dimethyl-1,3-dioxane-4,6-dione (2)

To a solution of 5-(methoxymethylene)-2,2-dimethyl-1,3-dioxane-4,6-dione (6.39 g, 34.35 mmol, 1 eq) in i-PrOH (60 mL) was added 4-chloro-3-fluoro-aniline (5 g, 34.35 mmol, 1 eq) at 50° C. The mixture was stirred at 80° C. for 3 h. LC-MS showed the starting material was consumed completely and desired product was detected. The reaction mixture was filtered and the filter cake was concentrated in vacuo. Compound 5-[(4-chloro-3-fluoro-anilino)methylene]-2,2-dimethyl-,3-dioxane-4,6-dione (9.03 g, 30.13 mmol, 87.72% yield) was obtained as a pale yellow solid. MS (M+H)⁺=300.1.

### 6-chloro-5-fluoro-quinolin-4-ol and 6-chloro-7-fluoro-quinolin-4-ol (3 and 3A)

A mixture of 5-[(4-chloro-3-fluoro-anilino)methylene]-2,2-dimethyl-1,3-dioxane-4,6-dione (700 mg, 2.34 mmol, 1 eq) in diphenyl ether (20 mL) was stirred at 250° C. for 1 h. LC-MS showed the starting material was consumed completely and desired product was detected. The mixture was allowed to 20° C. then poured into hexanes (10 mL). The resulting solid was collected by filtration. The crude product was purified by prep-HPLC (Waters Xbridge BEH C18 250*50 mm*10 um column; 10-30% acetonitrile in an a 10 mM ammonium bicarbonate solution in water, 10 min gradient). Compound 6-chloro-5-fluoro-quinolin-4-ol (200 mg, 969.99 umol, 4.61% yield) was obtained as a white solid. Compound 6-chloro-7-fluoro-quinolin-4-ol (500 mg, 2.25 mmol, 10.69% yield) was obtained as a white solid. ¹H NMR (400 MHz, DMSO-d6) δ 7.88-7.86 (m, 1H), 7.77-7.75 (, 1H), 7.40-7.38 (m, 1H), 6.01-5.99 (d, J=7.2, 1H). ¹H NMR (400 MHz, DMSO-d6) δ 8.15 (d, J=8.3 Hz, 1H), 8.00-7.91 (d, J=8.3 Hz, 1H), 7.50 (d, J=10.1 Hz, 1H), 6.07 (d, J=7.5 Hz, 1H). MS (M+H)⁺=198.2.

### 6-chloro-7-fluoro-4-hydroxy-quinoline-3-sulfonyl chloride (4A)

A mixture of 6-chloro-7-fluoro-quinolin-4-ol (500 mg, 2.53 mmol, 1 eq) in HSO₃Cl (6 mL) was stirred at 100° C. for 2 h. LC-MS showed the starting material was consumed completely and desired product was detected. The reaction mixture was filtered and the filter cake was concentrated in vacuo. Compound 6-chloro-7-fluoro-4-hydroxy-quinoline-3-sulfonyl chloride (600 mg, 1.66 mmol, 65.70% yield) was obtained as a pale yellow solid. MS (M+H)⁺=296.0.

### 6-chloro-7-fluoro-3-thiomorpholinosulfonyl-quinolin-4-ol (5A)

To a solution of 6-chloro-7-fluoro-4-hydroxy-quinoline-3-sulfonyl chloride (580 mg, 1.96 mmol, 1 eq) in DCM (7 mL) was added TEA (594.63 mg, 5.88 mmol, 817.92 uL, 3 eq) and thiomorpholine (404.24 mg, 3.92 mmol, 370.86 uL, 2 eq) at 0° C. The mixture was stirred at 25° C. for 2 h. LC-MS showed the starting material was consumed completely and desired product was detected. The reaction mixture was filtered and the filter cake was concentrated in vacuo. The crude product was purified by prep-HPLC (Waters Xbridge Prep OBD C18 150*40 mm*10 um column; 25-50% acetonitrile in an a 10 mM ammonium bicarbonate solution in water, 8 min gradient). Compound 6-chloro-7-fluoro-3-thiomorpholinosulfonyl-quinolin-4-ol (170 mg, 248.35 umol, 12.68% yield) was obtained as a white solid. MS (M+H)⁺=363.1.

### 4-[(4,6-dichloro-7-fluoro-3-quinolyl)sulfonyl]thiomorpholine (6A)

A mixture of 6-chloro-7-fluoro-3-thiomorpholinosulfonyl-quinolin-4-ol (150 mg, 413.42 umol, 1 eq) in POCl₃ (3 mL) was stirred at 120° C. for 4 hr under N₂ atmosphere. LC-MS showed the starting material was consumed completely and desired product was detected. The reaction mixture was concentrated to dryness to give the crude product. Then Ethyl acetate (5 mL) was added to it and poured into ice water (5 mL). The reaction mixture filtered and the filter cake was concentrated in vacuo. Compound 4-[(4,6-dichloro-7-fluoro-3-quinolyl)sulfonyl]thiomorpholine (20 mg, 49.44 umol, 11.96% yield) was obtained as a brown solid. MS (M+H)⁺=381.0.

### 5-chloro-2-[(6-chloro-7-fluoro-3-thiomorpholinosulfonyl-4-quinolyl)amino]benzoic acid (415A)

183. To a solution of 4-[(4,6-dichloro-7-fluoro-3-quinolyl)sulfonyl]thiomorpholine (18 mg, 47.21 umol, 1 eq) in EtOH (1 mL) and CH₃Cl (0.2 mL) was added 2-amino-5-chloro-benzoic acid (8.10 mg, 47.21 umol, 1 eq). The mixture was stirred at 80° C. for 2 h. LC-MS showed the starting material was consumed completely and desired product was detected. 5 mL of water was added to the reaction, the reaction mixture was extracted with Ethyl acetate (5 mL×2). The combined organic layers were washed with brine (5 mL), dried over Na₂SO₄ and concentrated to dryness to give a residue. The crude product was purified by prep-HPLC (Waters Xbridge BEH C18 100*30 mm*10 um column; 25-50% acetonitrile in an a 10 mM ammonium bicarbonate solution in water, 8 min gradient). The crude was purified by prep-HPLC (Phenomenex C18 80*40 mm*3 um column; 20-50% acetonitrile in an a 10 mM ammonium bicarbonate solution in water, 8 min gradient). Compound 5-chloro-2-[(6-chloro-7-fluoro-3-thiomorpholinosulfonyl-4-quinolyl)amino]benzoic acid (2.90 mg, 5.34 umol, 11.31% yield) was obtained as a yellow solid. ¹H NMR (400 MHz, METHANOL-d4) δ 9.16 (s, 1H), 8.07 (s, 1H), 7.93-7.83 (m, 2H), 7.34-7.23 (m, 1H), 6.64-6.56 (m, 1H), 3.48-3.41 (m, 4H), 2.60-2.47 (m, 4H). MS (M+H)⁺=516.0.

### Example 153—Synthesis of 416A

Synthetic scheme is provided in FIG. 39R.

### Synthesis of 5-[(3-bromo-4-chloro-anilino)methylene]-2,2-dimethyl-1,3-dioxane-4,6-dione (2)

To a solution of 5-(methoxymethylene)-2,2-dimethyl-1,3-dioxane-4,6-dione (2.70 g, 14.53 mmol, 1 eq) in i-PrOH (40 mL) was added 3-bromo-4-chloro-aniline (3 g, 14.53 mmol, 1 eq) at 50° C. The mixture was stirred at 80° C. for 3 h. LCMS showed the starting material was consumed completely and desired MS was detected. The reaction mixture was filtered and the filter cake was concentrated in vacuo. Compound 5-[(3-bromo-4-chloro-anilino)methylene]-2,2-dimethyl-1,3-dioxane-4,6-dione (4.8 g, 13.31 mmol, 91.61% yield) was obtained as a white solid. MS (M+H)⁺=360.0.

### Synthesis of 5-bromo-6-chloro-quinolin-4-ol (3) and 7-bromo-6-chloro-quinolin-4-ol (3 and 3A)

A mixture of 5-[(3-bromo-4-chloro-anilino)methylene]-2,2-dimethyl-1,3-dioxane-4,6-dione (660 mg, 1.83 mmol, 1 eq) in diphenyl ether (15 mL) was stirred at 250° C. for 1 h. LCMS showed the starting material was consumed completely and desired MS was detected. The crude reaction mixture (2 g scale) was combined to this batch for work-up. The mixture was allowed to 20° C. then poured into hexanes (40 mL). The resulting solid was collected by filtration and washed with hexanes. The residue was purified by prep-HPLC (Welch Xtimate C18 250*70 mm*10 um column; 20-45% acetonitrile in a 10 mM ammonium bicarbonate solution in water, 20 min gradient). Compound 5-bromo-6-chloro-quinolin-4-ol (350 mg, 1.35 mmol) was obtained as a white solid. Compound 7-bromo-6-chloro-quinolin-4-ol (620 mg, 2.40 mmol) was obtained as a white solid. ¹H NMR (400 MHz, DMSO-d6) δ 11.87 (br s, 1H), 7.85 (d, J=7.4 Hz, 1H), 7.78 (d, J=8.9 Hz, 1H), 7.54 (d, J=9.0 Hz, 1H), 6.07 (d, J=7.4 Hz, 1H). ¹H NMR (400 MHz, DMSO-d6) δ 8.13 (s, 1H), 7.98-7.95 (m, 2H), 6.08 (d, J=7.5 Hz, 1H). MS (M+H)⁺=260.0.

### Synthesis of 7-bromo-6-chloro-4-hydroxy-quinoline-3-sulfonyl chloride (4A)

A mixture of 7-bromo-6-chloro-quinolin-4-ol (600 mg, 2.32 mmol, 1 eq) in HSO₃Cl (6 mL) was stirred at 100° C. for 12 h. LCMS showed the starting material was consumed completely and desired MS was detected. The reaction mixture was poured into ice water and filtered. The filter cake was concentrated in vacuo. Compound 7-bromo-6-chloro-4-hydroxy-quinoline-3-sulfonyl chloride (810 mg, crude) was obtained as a pale yellow solid. MS (M+H)⁺=357.9

### Synthesis of 7-bromo-6-chloro-3-thiomorpholinosulfonyl-quinolin-4-ol (5A)

To a solution of 7-bromo-6-chloro-4-hydroxy-quinoline-3-sulfonyl chloride (800 mg, 2.24 mmol, 1 eq) in DCM (12 mL) was added Et₃N (680.25 mg, 6.72 mmol, 935.69 uL, 3 eq) and thiomorpholine (462.45 mg, 4.48 mmol, 424.26 uL, 2 eq). The mixture was stirred at 25° C. for 12 h. LCMS showed the starting material was consumed completely and desired MS was detected. The reaction mixture was concentrated to dryness to give the crude product. Compound 7-bromo-6-chloro-3-thiomorpholinosulfonyl-quinolin-4-ol (210 mg, crude) was obtained as a yellow solid. MS (M+H)⁺=424.9.

### Synthesis of 4-[(7-bromo-4,6-dichloro-3-quinolyl)sulfonyl]thiomorpholine (6A)

A mixture of 7-bromo-6-chloro-3-thiomorpholinosulfonyl-quinolin-4-ol (180 mg, 424.80 umol, 1 eq) in POCl₃ (3 mL) was stirred at 110° C. for 6 h. LCMS showed the starting material was consumed completely and desired MS was detected. The reaction mixture was concentrated to dryness to give the crude product. Then ethyl acetate (10 mL) was added to it and poured into ice water (10 mL). The reaction mixture was extracted with Ethyl acetate (10 mL*3). The combined organic layers were washed with brine (10 mL), dried over Na₂SO₄ and concentrated to dryness to give a residue. The crude product was purified by flash column (ISCO 20 g silica, 0-23% ethyl acetate in petroleum ether, gradient over 20 min). Compound 4-[(7-bromo-4,6-dichloro-3-quinolyl)sulfonyl]thiomorpholine (150 mg, crude) was obtained as a white solid. MS (M+H)⁺=442.9

### Synthesis of 2-[(7-bromo-6-chloro-3-thiomorpholinosulfonyl-4-quinolyl)amino]-5-chloro-benzoic acid (416A)

To a solution of 4-[(7-bromo-4,6-dichloro-3-quinolyl)sulfonyl]thiomorpholine (140 mg, 316.61 umol, 1 eq) in EtOH (3 mL) and CHCl₃ (0.6 mL) was added 2-amino-5-chloro-benzoic acid (108.65 mg, 633.23 umol, 2 eq). The mixture was stirred at 80° C. for 2 h. LCMS showed the starting material was consumed completely and desired MS was detected. The reaction mixture was concentrated to dryness to give the crude product. The crude product was purified by prep-HPLC (Phenomenex Luna 80*30 mm*3 um column; 45-80% acetonitrile in a 0.05% hydrochloric acid solution in water, 8 min gradient). Compound 2-[(7-bromo-6-chloro-3-thiomorpholinosulfonyl-4-quinolyl)amino]-5-chloro-benzoic acid (18.90 mg, 31.88 umol, 10.07% yield, 97.384% purity) was obtained as a yellow solid. ¹H NMR (400 MHz, METHANOL-d4) δ=9.19 (br d, J=1.8 Hz, 1H), 8.43 (d, J=1.8 Hz, 1H), 8.13 (br s, 1H), 7.80 (d, J=1.9 Hz, 1H), 7.45 (br d, J=8.8 Hz, 1H), 6.92 (dd, J=1.6, 8.7 Hz, 1H), 3.51 (br s, 4H), 2.68-2.54 (m, 4H). MS (M+H)⁺=575.9.

### Example 154—Synthesis of 417A

Synthetic scheme is provided in FIG. 39S.

### Synthesis of 5-[(4-chloro-2-methyl-anilino)methylene]-2,2-dimethyl-1,3-dioxane-4,6-dione (2)

To a solution of 5-(methoxymethylene)-2,2-dimethyl-1,3-dioxane-4,6-dione (1.31 g, 7.06 mmol, 1 eq) in i-PrOH (10 mL) was added 4-chloro-2-methyl-aniline (1 g, 7.06 mmol, 1 eq) at 50° C. and stirred at 80° C. for 2.5 h. LCMS showed the starting material was consumed completely and desired MS was detected. The reaction mixture was filtered and the filter cake was concentrated in vacuo. Compound 5-[(4-chloro-2-methyl-anilino)methylene]-2,2-dimethyl-1,3-dioxane-4,6-dione (1.43 g. 4.84 mmol, 68.47% yield) was obtained as a yellow solid. ¹H NMR (400 MHz, DMSO-d₆) δ=11.45 (br d, J=14.1 Hz, 1H), 8.77 (d, J=14.1 Hz, 1H), 7.85 (d, J=8.6 Hz, 1H), 7.60 (d, J=2.0 Hz, 1H), 7.52 (dd, J=2.1, 8.6 Hz, 1H), 2.50 (s, 3H), 1.86 (s, 6H). MS (M+H)⁺=297.1.

### Synthesis of 6-chloro-8-methyl-quinolin-4-ol (3)

A mixture of 5-[(4-chloro-2-methyl-anilino)methylene]-2,2-dimethyl-1,3-dioxane-4,6-dione (430 mg, 1.45 mmol, 1 eq) and diphenyl ether (4 mL) was heated to reflux at 250° C. for 1 h. LCMS showed the starting material was consumed completely and desired MS was detected. The mixture was allowed to 20° C. then poured into hexanes (5 mL). The resulting solid was collected by filtration and washed with hexanes. Compound 6-chloro-8-methyl-quinolin-4-ol (240 mg, 1.24 mmol, 28.41% yield) was obtained as a brown solid. ¹H NMR (400 MHz, DMSO-d6) δ=11.28 (br d, J=3.1 Hz, 1H), 7.95-7.81 (m, 2H), 7.59 (d, J=1.8 Hz, 1H), 6.12 (d, J=7.3 Hz, 1H), 2.54-2.50 (m, 3H). MS (M+H)⁺=194.1.

### Synthesis of 6-chloro-4-hydroxy-8-methyl-quinoline-3-sulfonyl chloride (4)

A mixture of 6-chloro-8-methyl-quinolin-4-ol (200 mg, 103.29 umol, 1 eq) and HSO₃Cl (1 mL) was stirred at 100° C. for 12 h. LCMS showed the starting material was consumed completely and desired MS was detected. The reaction mixture was poured into ice water and filtered. The filter cake was concentrated in vacuo. Compound 6-chloro-4-hydroxy-8-methyl-quinoline-3-sulfonyl chloride (150 mg, 51.35 umol, 24.86% yield) was obtained as a yellow solid. ¹H NMR (400 MHz, DMSO-d₆) δ=8.61 (s, 1H), 8.35 (s, 1H), 8.06 (d, J=2.3 Hz, 1H), 7.80 (d, J=1.5 Hz, 1H), 2.61 (s, 3H). MS (M+H)⁺=292.0.

### Synthesis of 6-chloro-8-methyl-3-thiomorpholinosulfonyl-quinolin-4-ol (5)

To a solution of 6-chloro-4-hydroxy-8-methyl-quinoline-3-sulfonyl chloride (150 mg, 104.23 umol, 1 eq) in DCM (5 mL) was added Et₃N (155.85 mg, 102.69 umol, 14.29 uL, 3 eq) and thiomorpholine (7.06 mg, 68.46 umol, 6.48 uL, 2 eq). The mixture was stirred at 25° C. for 12 h. LCMS showed the starting material was consumed completely and desired MS was detected. The reaction mixture was filtered and the filter cake was concentrated in vacuo. Compound 6-chloro-8-methyl-3-thiomorpholinosulfonyl-quinolin-4-ol (200 mg, 51.35 umol) was obtained as a yellow solid. MS (M+H)⁺=359.1.

### Synthesis of 4-[(4,6-dichloro-8-methyl-3-quinolyl)sulfonyl]thiomorpholine (6)

A mixture of 6-chloro-8-methyl-3-thiomorpholinosulfonyl-quinolin-4-ol (200 mg, 557.32 umol, 1 eq) and POCl₃ (4 ml) was stirred at 110° C. for 3 h. LCMS showed the starting material was consumed completely and desired MS was detected. The reaction mixture was concentrated to dryness to give the crude product. Then Ethyl acetate (5 mL) was added to it and poured into ice water (5 mL) and basified by sat. NaHCO₃ to pH=8-9 at 0° C. The reaction mixture was extracted with Ethyl acetate (5 mL*3). The combined organic layers were washed with brine (5 mL) and dried over Na₂SO₄ and concentrated to dryness to give residue. The crude product was purified by flash column (ISCO 20 g silica, 0-17% ethyl acetate in petroleum ether, gradient over 20 min). Compound 4-[(4,6-dichloro-8-methyl-3-quinolyl)sulfonyl]thiomorpholine (150 mg, 397.55 umol, 71.33% yield) was obtained as a white solid. ¹H NMR (400 MHz, CHLOROFORM-d) 6=9.33 (s, 1H), 8.26 (d, J=2.0 Hz, 1H), 7.71 (d, J=1.1 Hz, 1H), 3.70-3.66 (m, 4H), 2.82 (s, 3H), 2.72 (br d, J=4.4 Hz, 4H).

### Synthesis of 4-[(4,6-dichloro-8-methyl-3-quinolyl)sulfonyl]thiomorpholine (417A)

To a solution of 4-[(4,6-dichloro-8-methyl-3-quinolyl)sulfonyl]thiomorpholine (140 mg, 371.05 umol, 1 eq) in EtOH (2 mL) and CHCl₃ (0.4 mL) was added 2-amino-5-chloro-benzoic acid (127.33 mg, 742.10 umol, 2 eq). The mixture was stirred at 80° C. for 2 h. LCMS showed the starting material was consumed completely and desired MS was detected. The reaction mixture was concentrated to dryness to give the crude product. The crude product was purified by prep-HPLC (Phenomenex luna C18 80*40 mm*3 um column; 40-70% acetonitrile in an a 0.05% hydrochloric acid solution in water, 7 min gradient). Compound 5-chloro-2-[(6-chloro-8-methyl-3-thiomorpholinosulfonyl-4-quinolyl)amino]benzoic acid (54.80 mg, 106.94 umol, 28.82% yield, 100% purity) was obtained as a yellow solid. ¹H NMR (400 MHz, METHANOL-d4) δ=9.12 (s, 1H), 8.10 (d, J=2.6 Hz, 1H), 7.78 (d, J=1.1 Hz, 1H), 7.53 (d, J=2.1 Hz, 1H), 7.37 (dd, J=2.6, 8.8 Hz, 1H), 6.70 (d, J=8.9 Hz, 1H), 3.47 (1, J=5.1 Hz, 4H), 2.79 (s, 3H), 2.65-2.48 (m, 4H). MS (M+H)¹=512.0.

### Example 155—Synthesis of 418A

Synthetic scheme is provided in FIG. 39T.

### Synthesis of 5-[(2-bromo-4-chloro-anilino)methylene]-2,2-dimethyl-1,3-dioxane-4,6-dione (2)

To a solution of 5-(methoxymethylene)-2,2-dimethyl-1,3-dioxane-4,6-dione (2.70 g, 14.53 mmol, 1 eq) in i-PrOH (30 mL) was added 2-bromo-4-chloro-aniline (3 g, 14.53 mmol, 1 eq) at 50° C. and stirred at 80° C. for 2.5 h. LCMS showed the starting material was consumed completely and desired MS was detected. The reaction mixture was filtered and the filter cake was concentrated in vacuo. Compound 5-[(2-bromo-4-chloro-anilino)methylene]-2,2-dimethyl-1,3-dioxane-4,6-dione (4 g, 11.09 mmol, 76.34% yield) was obtained as a yellow solid. ¹H NMR (400 MHz, DMSO-d₆) δ=11.52 (br d, J=12.9 Hz, 1H), 8.74 (br d, J=13.1 Hz, 1H), 7.98-7.87 (m, 2H), 7.57 (dd, J=2.4, 8.8 Hz, 1H), 1.70 (s, 6H). MS (M+H)⁺=361.0.

### Synthesis of 8-bromo-6-chloro-quinolin-4-ol (3)

A mixture of 5-[(2-bromo-4-chloro-anilino)methylene]-2,2-dimethyl-1,3-dioxane-4,6-dione (670 mg, 1.86 mmol, 1 eq) and diphenyl ether (20 mL) was stirred at 250° C. for 1 h. LCMS showed the starting material was consumed completely and desired MS was detected. The mixture was allowed to 20° C. then poured into hexanes (15 mL). The resulting solid was collected by filtration and washed with hexanes. Compound 8-bromo-6-chloro-quinolin-4-ol (1.13 g, 4.37 mmol, 78.42% yield) was obtained as a yellow solid. ¹H NMR (400 MHz, DMSO-d6) δ=11.30 (br d, J=2.9 Hz, 1H), 8.14 (d, J=2.4 Hz, 1H), 8.05 (d, J=2.3 Hz, 1H), 7.88 (t, J=6.8 Hz, 1H), 6.16 (d, J=7.5 Hz, 1H) MS (M+H)⁺=258.0.

### Synthesis of 8-bromo-6-chloro-4-hydroxy-quinoline-3-sulfonyl chloride (4)

A mixture of 8-bromo-6-chloro-quinolin-4-ol (1 g, 3.87 mmol, 1 eq) and HSO₃Cl (10 mL) was stirred at 100° C. for 12 h. LCMS showed the starting material was consumed completely and desired MS was detected. The reaction mixture was poured into ice water and filtered. The filter cake was concentrated in vacuo. Compound 8-bromo-6-chloro-4-hydroxy-quinoline-3-sulfonyl chloride (1.2 g, 3.36 mmol, 86.89% yield) was obtained as a brown solid. ¹H NMR (400 MHz, DMSO-d6) δ=8.42 (s, 1H), 8.16 (d, J=2.3 Hz, 1H), 8.10 (d, J=2.3 Hz, 1H). MS (M+H)⁺=357.9.

### Synthesis of 8-bromo-6-chloro-3-thiomorpholinosulfonyl-quinolin-4-ol (5)

To a solution of 8-bromo-6-chloro-4-hydroxy-quinoline-3-sulfonyl chloride (1.2 g, 3.36 mmol, 1 eq) in DCM (15 mL) was added Et₃N (1.02 g, 10.08 mmol, 1.40 mL, 3 eq) and thiomorpholine (693.67 mg, 6.72 mmol, 636.39 uL, 2 eq). The mixture was stirred at 25° C. for 12 h. LCMS showed the starting material was consumed completely and desired MS was detected. The mixture was filtered and the filtrate was dried over in vacuo to afford the desired product. Compound 8-bromo-6-chloro-3-thiomorpholinosulfonyl-quinolin-4-ol (1.8 g, crude) was obtained as a brown oil. MS (M+H)⁺=357.9.

### Synthesis of 6-chloro-8-methoxy-3-thiomorpholinosulfonyl-quinolin-4-ol (6)

A mixture of 8-bromo-6-chloro-3-thiomorpholinosulfonyl-quinolin-4-ol (1 g, 2.36 mmol, 1 eq), CuI (898.92 mg, 4.72 mmol, 2 eq), NaOMe (5 M, 2.36 mL, 5 eq) in dioxane (40 mL) was degassed and purged with N₂ for 3 times, and then the mixture was stirred at 100° C. for 2 h under N₂ atmosphere. LCMS showed the starting material was consumed completely and desired MS was detected. The reaction mixture was filtered and 15 mL of water was added to the filtrate. The filtrate was extracted with Ethyl acetate (40 mL*3). The combined organic layers were washed with brine (30 mL) and dried over Na₂SO₄. The combined organic layer was concentrated to dryness to give residue. Compound 6-chloro-8-methoxy-3-thiomorpholinosulfonyl-quinolin-4-ol (2 g, crude) was obtained as a brown oil. MS (M+H)⁺=375.1.

### Synthesis of 4-[(4,6-dichloro-8-methoxy-3-quinolyl)sulfonyl]thiomorpholine (7)

A mixture of 6-chloro-8-methoxy-3-thiomorpholinosulfonyl-quinolin-4-ol (1.9 g, 5.07 mmol, 1 eq) and POCl₃ (15 mL) was stirred at 110° C. for 2 h. LCMS showed the starting material was consumed completely and desired MS was detected. The reaction mixture was concentrated to dryness to give the crude product. Then Ethyl acetate (20 mL) was added to it and poured into ice water (20 mL). The reaction mixture was extracted with Ethyl acetate (20 mL*3). The combined organic layers were washed with brine (20 mL) and dried over Na₂SO₄. The combined organic layer was concentrated to dryness to give residue. The crude product was purified by flash column (ISCO 20 g silica, 0-30% ethyl acetate in petroleum ether, gradient over 20 min). Compound 4-[(4,6-dichloro-8-methoxy-3-quinolyl)sulfonyl]thiomorpholine (130 mg, 330.53 umol, 6.52% yield) was obtained as a yellow solid. MS (M+H)⁺=393.0.

### Synthesis of 5-chloro-2-[(6-chloro-8-methoxy-3-thiomorpholinosulfonyl-4-quinolyl)amino]benzoic acid (418A)

To a solution of 4-[(4,6-dichloro-8-methoxy-3-quinolyl)sulfonyl]thiomorpholine (120 mg, 305.10 umol, 1 eq) in EtOH (2 mL) and CHCl₃ (0.4 mL) was added 2-amino-5-chloro-benzoic acid (104.70 mg, 610.21 umol, 2 eq). The mixture was stirred at 80° C. for 2 h. LCMS showed the starting material was consumed completely and desired MS was detected. The reaction mixture was concentrated to dryness to give the crude product. The crude product was purified by prep-HPLC (Phenomenex luna C18 80*40 mm*3 um column; 40-70% acetonitrile in an a 0.05% hydrochloric acid solution in water, 7 min gradient). Compound 5-chloro-2-[(6-chloro-8-methoxy-3-thiomorpholinosulfonyl-4-quinolyl)amino]benzoic acid (50.40 mg, 94.58 umol, 31.00% yield, 99.165% purity) was obtained as a yellow solid. 207. ¹H NMR (400 MHz, METHANOL-d₄) δ=9.02 (s, 1H), 8.13 (d, J=2.5 Hz, 1H), 7.48 (d, J=1.9 Hz, 1H), 7.44 (dd, J=2.6, 8.8 Hz, 1H), 7.15 (d, J=1.9 Hz, 1H), 6.90 (d, J=8.8 Hz, 1H), 4.15 (s, 3H), 3.50 (t, J=5.0 Hz, 4H), 2.60 (tq, J=5.0, 13.7 Hz, 4H). MS (M+H)⁺=528.1.

### Example 156—Synthesis of 419A

Synthetic scheme is provided in FIG. 39U.

### 5-[[4-chloro-2-(trifluoromethyl)anilino]methylene]-2,2-dimethyl-1,3-dioxane-4,6-dione (2)

A mixture of 5-(methoxymethylene)-2,2-dimethyl-1,3-dioxane-4,6-dione (4.76 g, 25.57 mmol, 1 eq) in i-PrOH (60 mL) was added 4-chloro-2-(trifluoromethyl)aniline (5 g, 25.57 mmol, 3.60 mL, 1 eq) at 50° C. The mixture was stirred at 80° C. for 3 h. LC-MS showed the starting material was consumed completely and desired mass was detected. The reaction mixture was filtered and the filter cake was concentrated in vacuo. Compound 5-[[4-chloro-2-(trifluoromethyl)anilino]methylene]-2,2-dimethyl-1,3-dioxane-4,6-dione (6.5 g, 18.46 mmol, 72.19% yield) was obtained as a brown solid. MS (M+H)⁺=350.1.

### 6-chloro-8-(trifluoromethyl)quinolin-4-ol (3)

A mixture of 5-[[4-chloro-2-(trifluoromethyl)anilino]methylene]-2,2-dimethyl-1,3-dioxane-4,6-dione (700 mg, 2.00 mmol, 1 eq) in diphenyl ether (20 mL) was stirred at 250° C. for 1 h. LC-MS showed the starting material was consumed completely and desired mass was detected. The mixture was allowed to 20° C. then poured into hexanes (10 mL). The resulting solid was collected by filtration and washed with hexanes. Compound 6-chloro-8-(trifluoromethyl)quinolin-4-ol (1 g, 4.04 mmol, 67.25% yield) was obtained as a brown solid. MS (M+H)⁺=248.2.

### 6-chloro-4-hydroxy-8-(trifluoromethyl)quinoline-3-sulfonyl chloride (4)

A mixture of 6-chloro-8-(trifluoromethyl)quinolin-4-ol (500 mg, 2.02 mmol, 1 eq) in HSO₃Cl (6 mL) was stirred at 100° C. for 2 h. LC-MS showed the starting material was consumed completely and desired mass was detected. The reaction mixture was filtered and the filter cake was concentrated in vacuo. Compound 6-chloro-4-hydroxy-8-(trifluoromethyl)quinoline-3-sulfonyl chloride (300 mg, 230.90 umol, 11.43% yield) was obtained as a pale yellow solid. MS (M+H)⁺=346.1.

### 6-chloro-3-thiomorpholinosulfonyl-8-(trifluoromethyl)quinolin-4-ol (5)

To a solution of 6-chloro-4-hydroxy-8-(trifluoromethyl)quinoline-3-sulfonyl chloride (280 mg, 808.99 umol, 1 eq) in DCM (4 mL) was added TEA (245.59 mg, 2.43 mmol, 337.81 uL, 3 eq) and thiomorpholine (166.95 mg, 1.62 mmol, 153.17 uL, 2 eq) at 0° C. The mixture was stirred at 25° C. for 2 h. LC-MS showed the starting material was consumed completely and desired mass was detected. The reaction mixture was filtered and the filter cake was concentrated in vacuo. Compound 6-chloro-3-thiomorpholinosulfonyl-8-(trifluoromethyl)quinolin-4-ol (500 mg, crude) was obtained as a brown solid. MS (M+H)⁺=413.1

### 4-[[4,6-dichloro-8-(trifluoromethyl)-3-quinolyl]sulfonyl]thiomorpholine (6)

A mixture of 6-chloro-3-thiomorpholinosulfonyl-8-(trifluoromethyl)quinolin-4-ol (400 mg, 968.91 umol, 1 eq) in POCl₃ (5 mL) was stirred at 120° C. for 4 hr under N₂ atmosphere. LC-MS showed the starting material was consumed completely and desired mass was detected. The reaction mixture was concentrated to dryness to give the crude product. Then Ethyl acetate (5 mL) was added to it and poured into ice water (5 mL). The reaction mixture was filtered and the filter cake was concentrated in vacuo. Compound 4-[[4,6-dichloro-8-(trifluoromethyl)-3-quinolyl]sulfonyl]thiomorpholine (200 mg, 444.91 umol, 45.92% yield) was obtained as a brown solid. MS (M+H)⁺=431.0

### 5-chloro-2-[[6-chloro-3-thiomorpholinosulfonyl-8-(trifluoromethyl)-4-quinolyl]amino]benzoic acid (419A)

To a solution of 4-[[4,6-dichloro-8-(trifluoromethyl)-3-quinolyl]sulfonyl]thiomorpholine (180 mg, 417.36 umol, 1 eq) in EtOH (3 mL) and CH₃Cl (0.6 mL) was added 2-amino-5-chloro-benzoic acid (107.42 mg, 626.04 umol, 1.5 eq). The mixture was stirred at 80° C. for 2 h. LC-MS showed the starting material remained and desired mass was detected. The mixture was concentrated to dryness to give a residue. The crude product was purified by prep-HPLC (Waters Xbridge Prep OBD C18 150*40 mm*10 um column; 25-55% acetonitrile in an a 10 mM ammonium bicarbonate solution in water, 8 min gradient). Compound 5-chloro-2-[[6-chloro-3-thiomorpholinosulfonyl-8-(trifluoromethyl)-4-quinolyl]amino]benzoic acid (21.7 mg, 37.41 umol, 8.96% yield) was obtained as a yellow solid. ¹H NMR (400 MHz, METHANOL-d4) 6=9.26 (s, 1H), 8.17 (s, 1H), 8.04 (s, 1H), 7.96 (d, J=2.1 Hz, 1H), 7.28-7.20 (m, 1H), 6.55 (d, J=8.6 Hz, 1H), 3.48-3.40 (m, 4H), 2.63-2.43 (m, 4H) MS (M+H)⁺=566.1

### Example 157—Synthesis of 420A

Synthetic scheme is provided in FIG. 39V.

### Synthesis of 5-[(2,4-dichloroanilino)methylene]-2,2-dimethyl-1,3-dioxane-4,6-dione (2)

To a solution of 5-(methoxymethylene)-2,2-dimethyl-1,3-dioxane-4,6-dione (2.30 g, 12.34 mmol, 1 eq) in i-PrOH (40 mL) was added 2,4-dichloroaniline (2 g, 12.34 mmol, 1 eq) at 50° C. Then the mixture was stirred at 80° C. for 3 h. LCMS showed the starting material was consumed completely and desired MS was detected. The reaction mixture was filtered and the filter cake was concentrated in vacuo. Compound 5-[(2,4-dichloroanilino)methylene]-2,2-dimethyl-1,3-dioxane-4,6-dione (2.9 g, 9.17 mmol, 74.31% yield) was obtained as a yellow solid. ¹H NMR (400 MHz, DMSO-d₆) δ=11.56 (br d, J=13.9 Hz, 1H), 8.76 (d, J=13.9 Hz, 1H), 7.96 (d, J=8.9 Hz, 1H), 7.82 (d, J=2.3 Hz, 1H), 7.53 (dd, J=2.3, 8.8 Hz, 1H), 1.69 (s, 6H). MS (M+H)⁺=317.1.

### Synthesis of 6,8-dichloroquinolin-4-ol (3)

A mixture of 5-[(2,4-dichloroanilino)methylene]-2,2-dimethyl-1,3-dioxane-4,6-dione (500 mg, 1.58 mmol, 1 eq) and DIPHENYL ETHER (15 mL) was stirred at 250° C. for 1 h. LCMS showed the starting material was consumed completely and desired MS was detected. The mixture was allowed to 20° C. then poured into hexanes (15 mL). The resulting solid was collected by filtration and washed with hexanes. Compound 6,8-dichloroquinolin-4-ol (880 mg, 4.11 mmol, 86.65% yield) was obtained as a brown solid. MS (M+H)⁺=214.1.

### Synthesis of 6,8-dichloro-4-hydroxy-quinoline-3-sulfonyl chloride (4)

A mixture of 6,8-dichloroquinolin-4-ol (850 mg, 3.97 mmol, 1 eq) and HSO₃Cl (1 mL) was stirred at 100° C. for 36 h. LCMS showed 10% of starting material remained, 34% of desired product was detected. The reaction mixture was poured into ice water and filtered. The filter cake was concentrated in vacuo. Compound 6,8-dichloro-4-hydroxy-quinoline-3-sulfonyl chloride (1.25 g, crude) was obtained as a brown solid. ¹H NMR (400 MHz, DMSO-d₆) δ=8.37 (s, 1H), 8.08-8.05 (m, 2H). MS (M+H)¹=311.9.

### Synthesis of 6,8-dichloro-3-thiomorpholinosulfonyl-quinolin-4-ol (5)

To a solution of 6,8-dichloro-4-hydroxy-quinoline-3-sulfonyl chloride (1.2 g, 3.84 mmol, 1 eq) in DCM (12 mL) was added Et₃N (1.17 g, 11.52 mmol, 1.60 mL, 3 eq) and thiomorpholine (792.32 mg, 7.68 mmol, 726.90 uL, 2 eq). The mixture was stirred at 25° C. for 12 h. LCMS showed the starting material was consumed completely and desired MS was detected. The mixture was filtered and the filtrate was dried over in vacuo to afford the desired product. Compound 6,8-dichloro-3-thiomorpholinosulfonyl-quinolin-4-ol (2.02 g, crude) was obtained as a brown solid. MS (M+H)⁺=379.0.

### Synthesis of 4-[(4,6,8-trichloro-3-quinolyl)sulfonyl]thiomorpholine (6)

A mixture of 6,8-dichloro-3-thiomorpholinosulfonyl-quinolin-4-ol (2 g, 5.27 mmol, 1 eq) and POCl₃ (15 mL) was stirred at 110° C. for 2 h. LCMS showed the starting material was consumed completely and desired MS was detected. The reaction mixture was concentrated to dryness to give the crude product. Then Ethyl acetate (30 mL) was added to it and poured into ice water (30 mL) and basified by sat. NaHCO₃ to pH=8-9 at 0° C. The reaction mixture was extracted with Ethyl acetate (30 mL*3). The combined organic layers were washed with brine (3 mL), dried over Na₂SO₄ and concentrated to dryness to give residue. The crude product was purified by flash column (ISCO 20 g silica, 0-10% ethyl acetate in petroleum ether, gradient over 20 min). Compound 4-[(4,6,8-trichloro-3-quinolyl)sulfonyl]thiomorpholine (330 mg, 829.71 umol, 15.73% yield) was obtained as a pale yellow solid. MS (M+H)⁺=397.0.

### Synthesis of 5-chloro-2-[(6,8-dichloro-3-thiomorpholinosulfonyl-4-quinolyl)amino]benzoic acid (420A)

To a solution of 4-[(4,6,8-trichloro-3-quinolyl)sulfonyl]thiomorpholine (200 mg, 502.86 umol, 1 eq) in EtOH (3 mL) and CHCl₃ (0.6 mL) was added 2-amino-5-chloro-benzoic acid (172.56 mg, 1.01 mmol, 2 eq). The mixture was stirred at 80° C. for 3 h. LCMS showed the starting material was consumed completely and desired MS was detected. The reaction mixture was concentrated to dryness to give the crude product. The crude product was purified by prep-HPLC (Phenomenex Luna 80*30 mm*3 um column; 40-70% acetonitrile in an a 0.05% hydrochloric acid solution in water, 8 min gradient). Compound 5-chloro-2-[(6,8-dichloro-3-thiomorpholinosulfonyl-4-quinolyl)amino]benzoic acid (29.20 mg, 53.70 umol, 10.68% yield, 97.984% purity) was obtained as a yellow solid. ¹H NMR (400 MHz, METHANOL-d₄) δ=9.21 (s, 1H), 8.07 (dd, J=2.4, 10.4 Hz, 2H), 7.65 (d, J=2.3 Hz, 1H), 7.34 (dd, J=2.6, 8.9 Hz, 1H), 6.62 (d, J=8.9 Hz, 1H), 3.43 (br t, J=4.1 Hz, 4H), 2.62-2.55 (m, 2H), 2.54-2.46 (m, 2H). MS (M+H)¹=533.9.

### Example 158—Synthesis of 421A

Synthetic scheme is provided in FIG. 39W.

### 5-[(4-chloro-2-fluoro-anilino)methylene]-2,2-dimethyl-1,3-dioxane-4,6-dione (2)

To a solution of 5-(methoxymethylene)-2,2-dimethyl-1,3-dioxane-4,6-dione (1.28 g, 6.87 mmol, 1 eq) in i-PrOH (10 mL) was added 4-chloro-2-fluoro-aniline (1 g, 6.87 mmol, 1 eq) at 50° C. and stirred at 80° C. for 2.5 hr. LCMS showed the starting material was consumed completely and desired MS was detected. The reaction mixture was filtered and the filter cake was concentrated in vacuo. Compound 5-[(4-chloro-2-fluoro-anilino)methylene]-2,2-dimethyl-1,3-dioxane-4,6-dione (1.1 g, 3.67 mmol, 53.43% yield) was obtained as a white solid. MS (M+H)⁺=300.1.

### 6-chloro-8-fluoro-quinolin-4-ol (3)

A mixture of 5-[(4-chloro-2-fluoro-anilino)methylene]-2,2-dimethyl-1,3-dioxane-4,6-dione (360 mg, 1.20 mmol, 1 eq) and diphenyl ether (12 mL) was stirred at 250° C. for 1 h. LC-MS showed the starting material was consumed completely and desired mass was detected. The mixture was allowed to 20° C. then poured into hexanes (10 mL). The resulting solid was collected by filtration and washed with hexanes. Compound 6-chloro-8-fluoro-quinolin-4-ol (370 mg, 1.87 mmol, 51.96% yield) was obtained as a brown solid. MS (M+H)⁺=198.2.

6-chloro-8-fluoro-4-hydroxy-quinoline-3-sulfonyl chloride (4)

A mixture of 6-chloro-8-fluoro-quinolin-4-ol (350 mg, 1.77 mmol, 1 eq) in HSO₃Cl (4 mL) was stirred at 100° C. for 12 h under N₂ atmosphere. LC-MS showed the starting material was consumed completely and desired mass was detected. The reaction mixture was filtered and the filter cake was concentrated in vacuo. Compound 6-chloro-8-fluoro-4-hydroxy-quinoline-3-sulfonyl chloride (340 mg, 1.15 mmol, 64.82% yield) was obtained as a black solid. MS (M+H)⁺=296.0.

### 6-chloro-8-fluoro-3-thiomorpholinosulfonyl-quinolin-4-ol (5)

To a solution of 6-chloro-8-fluoro-4-hydroxy-quinoline-3-sulfonyl chloride (320 mg, 1.08 mmol, 1 eq) in DCM (3 mL) was added TEA (328.07 mg, 3.24 mmol, 451.27 uL, 3 eq) and thiomorpholine (223.03 mg, 2.16 mmol, 204.61 uL, 2 eq) at 0° C. The mixture was stirred at 25° C. for 2 h. LC-MS showed the starting material was consumed completely and desired mass was detected. The reaction mixture was filtered and the filter cake was concentrated in vacuo. Compound 6-chloro-8-fluoro-3-thiomorpholinosulfonyl-quinolin-4-ol (300 mg, 826.84 umol, 76.51% yield) was obtained as a brown solid. MS (M+H)⁺=363.1.

### 4-[(4,6-dichloro-8-fluoro-3-quinolyl)sulfonyl]thiomorpholine (6)

A mixture of 6-chloro-8-fluoro-3-thiomorpholinosulfonyl-quinolin-4-ol (35 mg, 96.46 umol, 1 eq) in POCl₃ (1 mL) was stirred at 120° C. for 4 hr under N₂ atmosphere. LC-MS showed the starting material was consumed completely and desired mass was detected. The reaction mixture was concentrated to dryness to give the crude product. Then Ethyl acetate (5 mL) was added to it and then the mixture was poured into ice water (8 mL). The reaction was filtered and the filter cake was concentrated in vacuo. Compound 4-[(4,6-dichloro-8-fluoro-3-quinolyl)sulfonyl]thiomorpholine (36 mg, 94.42 umol, 97.88% yield) was obtained as a brown solid. MS (M+H)⁺=381.1.

### 5-chloro-2-[(6-chloro-8-fluoro-3-thiomorpholinosulfonyl-4-quinolyl)amino]benzoic acid (421A)

To a solution of 4-[(4,6-dichloro-8-fluoro-3-quinolyl)sulfonyl]thiomorpholine (30 mg, 78.68 umol, 1 eq) in CHCl₃ (0.2 mL) and EtOH (1 mL) was added 2-amino-5-chloro-benzoic acid (20.25 mg, 118.03 umol, 1.5 eq). The mixture was stirred at 80° C. for 2 h. LC-MS showed the starting material was consumed completely and desired mass was detected. 5 mL of water was added to the reaction, the reaction mixture was extracted with Ethyl acetate (5 mL×3). The combined organic layers were washed with brine (5 mL) and dried over Na₂SO₄ and concentrated to dryness to give residue. The crude product was purified by prep-HPLC (Phenomenex Luna 80*30 mm*3 um column; 20-50% acetonitrile in an a 0.05% hydrochloricacid solution in water, 8 min gradient). Compound 5-chloro-2-[(6-chloro-8-fluoro-3-thiomorpholinosulfonyl-4-quinolyl)amino]benzoic acid (3.8 mg, 7.36 umol, 9.35% yield) was obtained as a yellow solid. ¹H NMR (400 MHz, METHANOL-d4) δ=9.17 (s, 1H), 8.11 (s, 1H), 7.77-7.74 (d, J=10 Hz, 1H), 7.50 (s, 1H), 7.40-7.37 (m, 1H), 6.75-6.69 (m, 1H), 3.47 (br t, J=4.5 Hz, 4H), 2.63-2.51 (m, 4H). MS (M+H)⁺=516.0.

### Example 159—Synthesis of 422A

Synthetic scheme is provided in FIG. 39X.

### 5-[(2-bromo-4-chloro-anilino)methylene]-2,2-dimethyl-1,3-dioxane-4,6-dione (2)

To a solution of 5-(methoxymethylene)-2,2-dimethyl-1,3-dioxane-4,6-dione (901.65 mg, 4.84 mmol, 1 eq) in i-PrOH (10 mL) was added 2-bromo-4-chloro-aniline (1 g, 4.84 mmol, 1 eq) at 50° C. and then the mixture was stirred at 80° C. for 2.5 hr. LCMS showed the starting material was consumed completely and desired MS was detected. The reaction mixture was filtered and the filter cake was concentrated in vacuo. Compound 5-[(2-bromo-4-chloro-anilino)methylene]-2,2-dimethyl-1,3-dioxane-4,6-dione (0.9 g, 2.50 mmol, 51.53% yield) was obtained as a yellow solid. MS (M+H)⁺=360.0

### 8-bromo-6-chloro-quinolin-4-ol (3)

A mixture of 5-[(2-bromo-4-chloro-anilino)methylene]-2,2-dimethyl-1,3-dioxane-4,6-dione (280 mg, 776.51 umol, 1 eq) and diphenyl ether (8 mL) was stirred at 250° C. for 1 h. LC-MS showed the starting material was consumed completely and desired mass was detected. The mixture was allowed to 20° C. then poured into hexanes (10 mL). The resulting solid was collected by filtration and washed with hexanes. Compound 8-bromo-6-chloro-quinolin-4-ol (460 mg, 1.78 mmol, 76.39% yield) was obtained as a brown solid. MS (M+H)⁺=258.1.

### 8-bromo-6-chloro-4-hydroxy-quinoline-3-sulfonyl chloride (4)

A mixture of 8-bromo-6-chloro-quinolin-4-ol (440 mg, 1.70 mmol, 1 eq) in HSO₃Cl (4 mL) was stirred at 100° C. for 12 h under N₂ atmosphere. LC-MS showed the starting material remained and desired mass was detected. The reaction mixture was filtered and the filter cake was concentrated in vacuo. Compound 8-bromo-6-chloro-4-hydroxy-quinoline-3-sulfonyl chloride (410 mg, 1.15 mmol, 67.47% yield) was obtained as a brown solid. MS (M+H)⁺=356.0

### 8-bromo-6-chloro-3-thiomorpholinosulfonyl-quinolin-4-ol (5)

To a solution of 8-bromo-6-chloro-4-hydroxy-quinoline-3-sulfonyl chloride (400 mg, 1.12 mmol, 1 eq) in DCM (4 mL) was added TEA (340.12 mg, 3.36 mmol, 467.85 uL, 3 eq) and thiomorpholine (231.22 mg, 2.24 mmol, 212.13 uL, 2 eq) at 0° C. The mixture was stirred at 25° C. for 2 h. LC-MS showed the starting material was consumed completely and desired mass was detected. The reaction mixture was filtered and the filter cake was concentrated in vacuo. Compound 8-bromo-6-chloro-3-thiomorpholinosulfonyl-quinolin-4-ol (310 mg, 731.59 umol, 65.30% yield) was obtained as a brown solid. MS (M+H)⁺=423.0.

### 4-[(8-bromo-4,6-dichloro-3-quinolyl)sulfonyl]thiomorpholine (6)

A mixture of 8-bromo-6-chloro-3-thiomorpholinosulfonyl-quinolin-4-ol (290 mg, 684.39 umol, 1 eq) in POCl₃ (3 mL) was stirred at 120° C. for 4 hr under N₂ atmosphere. LC-MS showed the starting material was consumed completely and desired mass was detected. The reaction mixture was concentrated to dryness to give the crude product. Then Ethyl acetate (5 mL) was added to it and then the mixture was poured into ice water (8 mL). The reaction was filtered and the filter cake was concentrated in vacuo. The crude product was purified by flash column (ISCO 10 g silica, 20-30% ethyl acetate in petroleum ether, gradient over 10 min). Compound 4-[(8-bromo-4,6-dichloro-3-quinolyl)sulfonyl]thiomorpholine (220 mg, 497.54 umol, 72.70% yield) was obtained as a yellow solid. MS (M+H)⁺=441.0.

### 2-[(8-bromo-6-chloro-3-thiomorpholinosulfonyl-4-quinolyl)amino]-5-chloro-benzoic acid (422A)

To a solution of 4-[(8-bromo-4,6-dichloro-3-quinolyl)sulfonyl]thiomorpholine (200 mg, 452.31 umol, 1 eq) in EtOH (4 mL) and CHCl₃ (0.8 mL) was added 2-amino-5-chloro-benzoic acid (155.21 mg, 904.61 umol, 2 eq). The mixture was stirred at 80° C. for 2 h. LC-MS showed the starting material was consumed completely and desired mass was detected. 2 mL of water was added to the reaction, the reaction mixture was extracted with Ethyl acetate (5 mL×3). The combined organic layers were washed with brine (2 mL) and dried over Na₂SO₄ and concentrated to dryness to give a residue. The crude product was purified by flash column (ISCO 10 g silica, 20-30% ethyl acetate in petroleum ether, gradient over 10 min). The crude product was purified by prep-HPLC (Waters Xbridge Prep OBD C18 150*40 mm*10 um column; 25-55% acetonitrile in an a 10 mM ammonium bicarbonate solution in water, 8 min gradient) to give crude product which was twice purified by prep-HPLC (Phenomenex Luna C18 75*30 mm*3 um column; 50-80% acetonitrile in an a 0.225% formic acid solution in water, 8 min gradient). Compound 2-[(8-bromo-6-chloro-3-thiomorpholinosulfonyl-4-quinolyl)amino]-5-chloro-benzoic acid (2.60 mg, 4.50 umol, 9.96e-1% yield) was obtained as a pale yellow solid. ¹H NMR (400 MHz, METHANOL-d4) δ=9.24 (s, 1H), 8.24 (s, 1H), 8.09 (d, J=2.6 Hz, 1H), 7.72 (d, J=2.3 Hz, 1H), 7.33 (dd, J=2.6, 8.9 Hz, 1H), 6.60-6.55 (d, J=8.8 Hz, 1H), 3.45-3.43 (m, 4H), 2.64-2.46 (m, 4H). MS (M+H)⁺=575.8.

### Example 160—Synthesis of 423A

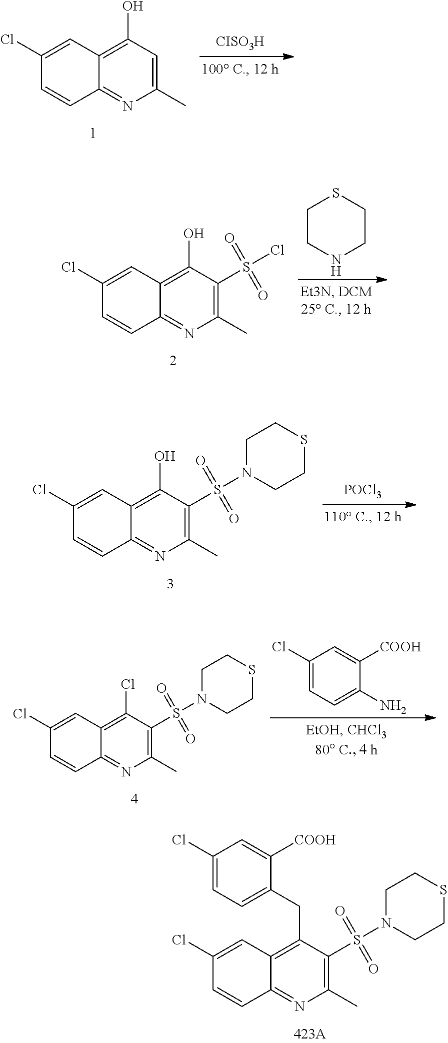

### Synthesis of 6-chloro-4-hydroxy-2-methyl-quinoline-3-sulfonyl chloride (2)

A mixture of 6-chloro-2-methyl-quinolin-4-ol (1 g, 5.16 mmol, 1 eq) and HSO₃Cl (10 mL) was stirred at 100° C. for 12 h. LCMS showed the starting material was consumed completely and desired MS was detected. The reaction mixture was poured into ice water and filtered. The filter cake was concentrated in vacuo. Compound 6-chloro-4-hydroxy-2-methyl-quinoline-3-sulfonyl chloride (940 mg, crude) was obtained as a brown solid. MS (M+H)⁺=292.0.

### Synthesis of 6-chloro-2-methyl-3-thiomorpholinosulfonyl-quinolin-4-ol (3)

To a solution of 6-chloro-4-hydroxy-2-methyl-quinoline-3-sulfonyl chloride (900 mg, 3.08 mmol, 1 eq) in DCM (15 mL) was added Et₃N (935.21 mg, 9.24 mmol, 1.29 mL, 3 eq) and thiomorpholine (635.77 mg, 6.16 mmol, 583.28 uL, 2 eq). The mixture was stirred at 25° C. for 12 h. LCMS showed the starting material was consumed completely and desired MS was detected. The reaction mixture was filtered and the filtrate was concentrated in vacuo. Compound 6-chloro-2-methyl-3-thiomorpholinosulfonyl-quinolin-4-ol (1.45 g, crude) was obtained as a brown oil. MS (M+H)⁺=359.1.

### 3. Synthesis of 4-[(4,6-dichloro-2-methyl-3-quinolyl)sulfonyl]thiomorpholine (4)

268. A mixture of 6-chloro-2-methyl-3-thiomorpholinosulfonyl-quinolin-4-ol (1.4 g, 3.90 mmol, 1 eq) and POCl₃ (10 mL) was stirred at 110° C. for 2 h. LCMS showed the starting material was consumed completely and desired MS was detected. The reaction mixture was concentrated to dryness to give the crude product. Then Ethyl acetate (15 mL) was added to it and poured into ice water (15 mL). The reaction mixture was extracted with Ethyl acetate (15 mL*3). The combined organic layers were washed with brine (10 mL), dried over Na₂SO₄ and concentrated to dryness to give residue. The crude product was purified by flash column (ISCO 20 g silica, 0-23% ethyl acetate in petroleum ether, gradient over 20 min). Compound 4-[(4,6-dichloro-2-methyl-3-quinolyl)sulfonyl]thiomorpholine (130 mg, 344.55 umol, 8.83% yield) was obtained as a pale yellow solid. ¹H NMR (400 MHz, DMSO-d₆) δ=8.38 (d, J=2.3 Hz, 1H), 8.10-8.05 (m, 1H), 8.03-7.98 (m, 1H), 3.58 (td, J=2.5, 4.8 Hz, 4H), 2.97 (s, 3H), 2.70-2.63 (m, 4H). MS (M+H)⁺=377.0.

### Synthesis of 5-chloro-2-[(6-chloro-2-methyl-3-thiomorpholinosulfonyl-4-quinolyl)amino]benzoic acid (423A)

To a solution of 4-[(4,6-dichloro-2-methyl-3-quinolyl)sulfonyl]thiomorpholine (120 mg, 318.04 umol, 1 eq) in EtOH (2 mL) and CHCl₃ (0.4 mL) was added 2-amino-5-chloro-benzoic acid (109.14 mg, 636.08 umol, 2 eq). The mixture was stirred at 80° C. for 4 h. LCMS showed the starting material was consumed completely and desired MS was detected. The reaction mixture was concentrated to dryness to give the crude product. The crude product was purified by prep-HPLC (Phenomenex luna C18 80*40 mm*3 um column; 50-80% acetonitrile in an a 0.05% hydrochloric acid solution in water, 7 min gradient). Compound 5-chloro-2-[(6-chloro-2-methyl-3-thiomorpholinosulfonyl-4-quinolyl)amino]benzoic acid (115.10 mg, 224.62 umol, 70.62% yield, 100% purity) was obtained as a yellow solid. ¹H NMR (400 MHz, METHANOL-d₄) δ=8.16 (d, J=2.5 Hz, 1H), 8.00-7.93 (m, 2H), 7.57 (s, 1H), 7.48 (dd, J=2.6, 8.8 Hz, 1H), 7.01 (d, J=8.8 Hz, 1H), 3.59 (t, J=5.1 Hz, 4H), 3.07 (s, 3H), 2.70-2.57 (m, 4H). MS (M+H)⁺=512.0.

### Example 161—Synthesis of 425A

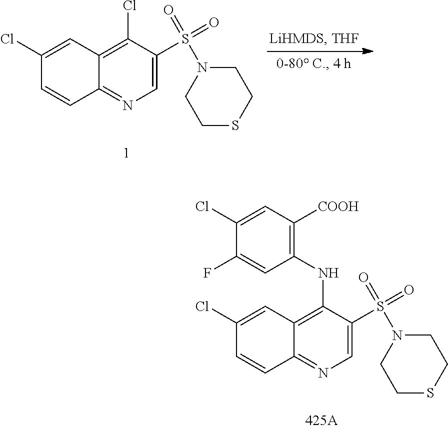 [SMILES: O=S(=O)(c1cnc2ccc(Cl)cc2c1Cl)N1CCSCC1 ; O=C(O)c1cc(Cl)c(F)cc1Nc1c(S(=O)(=O)N2CCSCC2)cnc2ccc(Cl)cc12]

### Synthesis of 5-chloro-2-[(6-chloro-3-thiomorpholinosulfonyl-4-quinolyl)amino]-4-fluoro-benzoic acid (425A)

To a solution of 2-amino-5-chloro-4-fluoro-benzoic acid (73.06 mg, 385.38 umol, 2 eq) in THF (1 mL) was added drop wise LiHMDS (1 M, 578.06 uL, 3 eq) at 0° C. and stirred at 20° C. for 1 h. Then 4-[(4,6-dichloro-3-quinolyl)sulfonyl]thiomorpholine (70 mg, 192.69 umol, 1 eq) in THF (0.5 mL) was added dropwise to the above mixture at 0° C. and stirred at 80° C. for 4 h under N₂ atmosphere. LCMS showed 20% of starting material remained, 15% of desired product was detected. 3 mL of sat·NH₄Cl was added slowly to the reaction in the ice bath, the reaction mixture was extracted with Ethyl acetate (3 mL*3). The combined organic layers were dried over Na₂SO₄ and concentrated to dryness to give residue. The crude product was purified by prep-HPLC (Phenomenex Luna 80*30 mm*3 um column; 50-80% acetonitrile in an a 0.05% hydrochloric acid solution in water, 8 min gradient). Compound 5-chloro-2-[(6-chloro-3-thiomorpholinosulfonyl-4-quinolyl)amino]-4-fluoro-benzoic acid (4.80 mg, 9.00 umol, 4.67% yield, 96.87% purity) was obtained as a yellow solid. ¹H NMR (400 MHz, METHANOL-d₄) δ=9.25 (s, 1H), 8.26 (d, J=8.3 Hz, 1H), 8.13 (d, J=9.0 Hz, 1H). 7.98 (dd, J=2.2, 9.1 Hz, 1H), 7.75 (d, J=2.1 Hz, 1H), 6.76 (d, J=10.8 Hz, 1H), 3.50 (td, J=3.5, 6.6 Hz, 4H), 2.67-2.54 (m, 4H). MS (M+H)⁺=516.0.

### Example 162—Synthesis of 426A

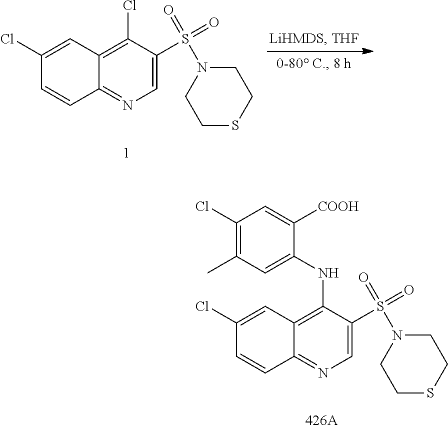 [SMILES: O=S(=O)(c1cnc2ccc(Cl)cc2c1Cl)N1CCSCC1 ; Cc1cc(Nc2c(S(=O)(=O)N3CCSCC3)cnc3ccc(Cl)cc23)c(C(=O)O)cc1Cl]

### Synthesis of 5-chloro-2-[(6-chloro-3-thiomorpholinosulfonyl-4-quinolyl)amino]-4-methyl-benzoic acid (426A)

To a solution of 2-amino-5-chloro-4-methyl-benzoic acid (80.00 mg, 431.02 umol, 1 eq) in THF (1.5 mL) was added dropwised LiHMDS (1 M, 1.29 mL, 3 eq) at 0° C. and stirred at 20° C. for 1 h. Then 4-[(4,6-dichloro-3-quinolyl)sulfonyl]thiomorpholine (313.16 mg, 862.04 umol, 2 eq) in THF (0.5 mL) was added drop wise to the above mixture at 0° C. and stirred at 80° C. for 7 h under N₂ atmosphere. LCMS showed the starting material was consumed completely and desired MS was detected. 3 mL of sat·NH₄Cl was added slowly to the reaction in the ice bath, the reaction mixture was extracted with Ethyl acetate (3 mL*3). The combined organic layers were dried over Na₂SO₄ and concentrated to dryness to give residue. The crude product was purified by prep-HPLC (Phenomenex Luna 80*30 mm*3 um column; 35-65% acetonitrile in an a 0.05% hydrochloric acid solution in water, 8 min gradient). Compound 5-chloro-2-[(6-chloro-3-thiomorpholinosulfonyl-4-quinolyl)amino]-4-methyl-benzoic acid (36.15 mg, 69.94 umol, 16.23% yield, 99.136% purity) was obtained as a yellow solid. ¹H NMR (400 MHz, METHANOL-d₄) δ=9.22 (s, 1H), 8.16 (s, 1H), 8.09-8.05 (m, 1H), 8.03-7.98 (m, 1H), 7.64 (d, J=2.1 Hz, 1H), 7.14 (s, 1H), 3.59 (t, J=5.1 Hz, 4H), 2.71-2.62 (m, 4H), 2.29 (s, 3H). MS (M+H)⁺=512.0.

### Example 163—Synthesis of 427A

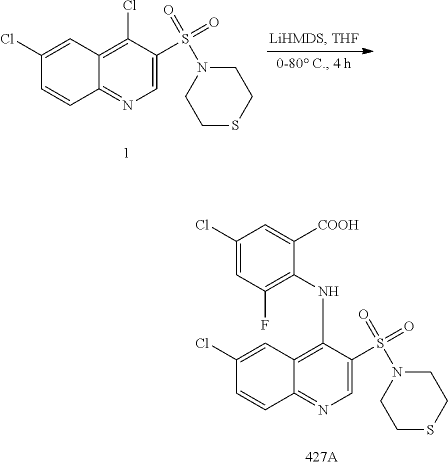 [SMILES: O=S(=O)(c1cnc2ccc(Cl)cc2c1Cl)N1CCSCC1 ; O=C(O)c1cc(Cl)cc(F)c1Nc1c(S(=O)(=O)N2CCSCC2)cnc2ccc(Cl)cc12]

### Synthesis of 5-chloro-2-[(6-chloro-3-thiomorpholinosulfonyl-4-quinolyl)amino]-3-fluoro-benzoic acid (427A)

To a solution of 2-amino-5-chloro-3-fluoro-benzoic acid (73.06 mg, 385.38 umol, 2 eq) in THF (1 mL) was added dropwise LiHMDS (1 M, 578.06 uL, 3 eq) at 0° C. and stirred at 20° C. for 1 h. Then 4-[(4,6-dichloro-3-quinolyl)sulfonyl]thiomorpholine (70 mg, 192.69 umol, 1 eq) in THF (0.5 mL) was added dropwise to the above mixture at 0° C. and stirred at 80° C. for 3 h under N₂ atmosphere. LCMS showed 17% of starting material remained, 22% of desired product was detected. 3 mL of sat·NH₄Cl was added slowly to the reaction in the ice bath, the reaction mixture was extracted with Ethyl acetate (3 mL*3). The combined organic layers were dried over Na₂SO₄ and concentrated to dryness to give residue. The crude product was purified by prep-HPLC (Phenomenex Luna 80*30 mm*3 um column; 50-80% acetonitrile in an a 0.05% hydrochloric acid solution in water, 8 min gradient). Compound 5-chloro-2-[(6-chloro-3-thiomorpholinosulfonyl-4-quinolyl)amino]-3-fluoro-benzoic acid (2.00 mg, 3.87 umol, 2.01% yield) was obtained as a yellow solid. ¹H NMR (400 MHz, METHANOL-d₄) δ=9.15 (s, 1H), 8.09-8.02 (m, 2H), 8.00-7.93 (m, 1H), 7.65-7.57 (m, 2H), 3.61-3.56 (m, 4H), 2.70-2.63 (m, 4H). MS (M+H)⁺=516.0.

### Example 164—Synthesis of 428A

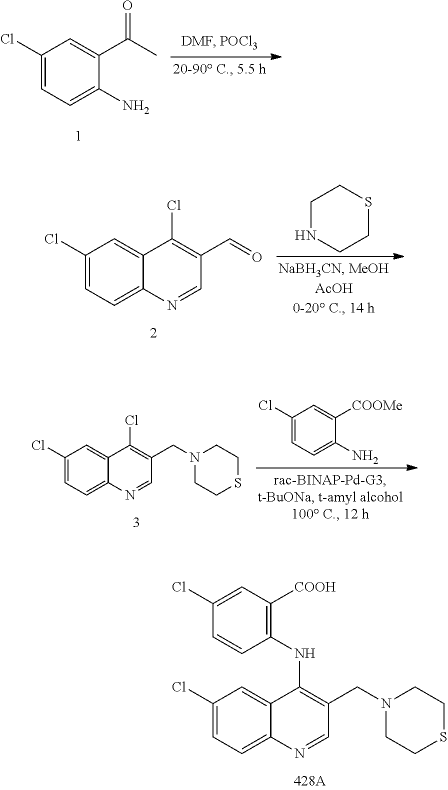 [SMILES: CC(=O)c1cc(Cl)ccc1N ; O=Cc1cnc2ccc(Cl)cc2c1Cl ; C1CSCCN1 ; Clc1ccc2ncc(CN3CCSCC3)c(Cl)c2c1 ; COC(=O)c1cc(Cl)ccc1N ; O=C(O)c1cc(Cl)ccc1Nc1c(CN2CCSCC2)cnc2ccc(Cl)cc12]

### Synthesis of 4,6-dichloroquinoline-3-carbaldehyde (2)

1-(2-amino-5-chloro-phenyl)ethanone (2 g, 11.79 mmol. 1 eq) was added to DMF (10 mL) and POCl₃ (16.50 g, 107.61 mmol, 10 mL, 9.13 eq) in portions. Then the mixture was purged with N₂ and stirred at 90° C. for 5 h under N₂ atmosphere. LCMS showed the starting material was consumed completely and desired MS was detected. The reaction mixture was concentrated to dryness to give the crude product. Then Ethyl acetate (30 mL) was added to it and poured into ice water (30 mL) and basified by sat. NaHCO₃ to pH=8-9 at 0° C. The reaction mixture was extracted with Ethyl acetate (30 mL*3). The combined organic layers were washed with brine (15 mL) and dried over Na₂SO₄. The combined organic layer was concentrated to dryness to give residue. The crude product was purified by flash column (ISCO 20 g silica, 0-23% ethyl acetate in petroleum ether, gradient over 20 min). Compound 4,6-dichloroquinoline-3-carbaldehyde (840 mg, 3.72 mmol, 31.51% yield) was obtained as a white solid. ¹H NMR (400 MHz, CHLOROFORM-d) 6=10.71 (s, 1H), 9.26 (s, 1H), 8.38 (d, J=2.3 Hz, 1H), 8.13 (d, J=9.0 Hz, 1H), 7.85 (dd, J=2.3, 8.9 Hz, 1H). MS (M+H)⁺=226.1.

### Synthesis of 4-[(4,6-dichloro-3-quinolyl)methyl]thiomorpholine (3)

To a solution of thiomorpholine (748.59 mg, 7.25 mmol, 686.78 μL, 2 eq) in MeOH (10 mL) was added 4,6-dichloroquinoline-3-carbaldehyde (820 mg, 3.63 mmol, 1 eq) and AcOH until pH=5. Then the mixture was stirred at 20° C. for 2 h. Then NaBH₃CN (455.90 mg, 7.25 mmol, 2 eq) was added to the above mixture at 0° C. in portions and stirred at 20° C. for 12 h. LCMS showed the starting material was consumed completely and desired MS was detected. Then the mixture was filtered and the filter cake was dried over in vacuo to afford the desired product. Compound 4-[(4,6-dichloro-3-quinolyl)methyl]thiomorpholine (850 mg, 2.71 mmol, 74.81% yield) was obtained as a white solid. ¹H NMR (400 MHz, DMSO-d₆) δ=8.95 (s, 1H), 8.21 (d, J=2.3 Hz, 1H), 8.11 (d, J=9.0 Hz, 1H), 7.88 (dd, J=2.3, 9.0 Hz, 1H), 3.85 (s, 2H), 2.76-2.71 (m, 4H), 2.65-2.61 (m, 4H). MS (M+H)⁺=313.0.

### Synthesis of 5-chloro-2-[[6-chloro-3-(thiomorpholinomethyl)-4-quinolyl]amino]benzoic acid (428A)

A mixture of 4-[(4,6-dichloro-3-quinolyl)methyl]thiomorpholine (200 mg, 638.48 umol, 1 eq), methyl 2-amino-5-chloro-benzoate (118.51 mg, 638.48 umol, 1 eq), t-BuONa (2 M, 638.48 uL, 2 eq), rac-BINAP-Pd-G3 (63.36 mg, 63.85 umol, 0.1 eq) in tert-amyl alcohol (3 mL) was degassed and purged with N₂ for 3 times, and then the mixture was stirred at 100° C. for 12 h under N₂ atmosphere. LCMS showed the starting material was consumed completely and desired MS was detected. The reaction mixture was filtered and the filtrate was concentrated in vacuo. The reaction mixture was purified by prep-HPLC (Waters Xbridge Prep OBD C18 150*40 mm*10 um column; 5-35% acetonitrile in an a 10 mM ammonium hydroxide solution in water, 8 min gradient). Compound 5-chloro-2-[[6-chloro-3-(thiomorpholinomethyl)-4-quinolyl]amino]benzoic acid (11.10 mg, 24.40 umol, 3.82% yield, 98.54% purity) was obtained as a yellow solid. ¹H NMR (400 MHz, DMSO-d₆) δ=8.84 (s, 1H), 8.06 (d, J=9.0 Hz, 1H), 7.90 (d, J=2.6 Hz, 1H), 7.73 (dd, J=2.4, 9.0 Hz, 1H), 7.60 (d, J=2.3 Hz, 1H), 7.27 (dd, J=2.7, 8.9 Hz, 1H), 6.29 (d, J=9.0 Hz, 1H), 3.78-3.65 (m, 1H), 3.49 (br d, J=12.8 Hz, 1H), 2.78-2.54 (m, 8H). MS (M+H)⁺=448.1.

### Example 165—Synthesis of 429A

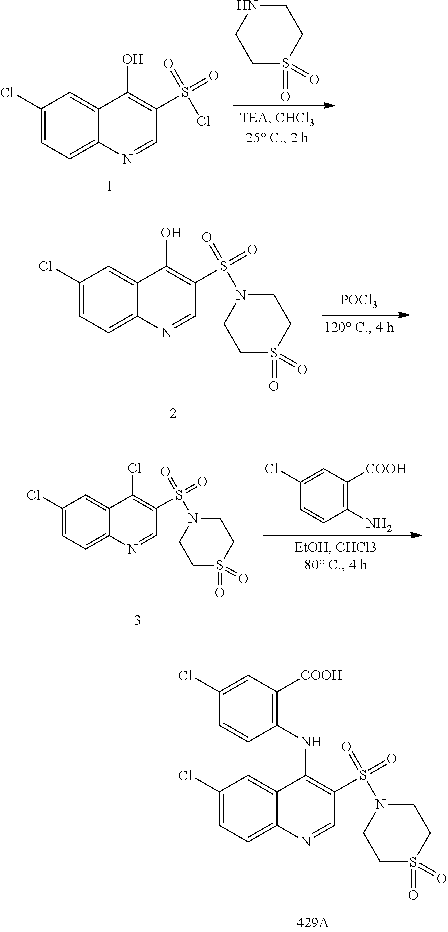

### Synthesis of 6-chloro-3-[(1,1-dioxo-1,4-thiazinan-4-yl)sulfonyl]quinolin-4-ol (2)

To a solution of 6-chloro-4-hydroxy-quinoline-3-sulfonyl chloride (500 mg, 1.80 mmol, 1 eq) in DCM (5 mL) was added 1,4-thiazinane 1,1-dioxide (486.08 mg, 3.60 mmol, 2 eq), Et₃N (545.76 mg, 5.39 mmol, 750.71 μL, 3 eq). The mixture was stirred at 25° C. for 4 h. LCMS showed the starting material was consumed completely and desired MS was detected. The reaction mixture was filtered and the filter cake was concentrated in vacuo. Compound 6-chloro-3-[(1,1-dioxo-1,4-thiazinan-4-yl)sulfonyl]quinolin-4-ol (200 mg, 530.74 umol, 29.52% yield) was obtained as a yellow solid. ¹H NMR (400 MHz, DMSO-d6) δ=8.59 (s, 1H), 8.11 (d, J=2.3 Hz, 1H), 7.87-7.81 (m, 1H), 7.78-7.71 (m, 1H), 3.74 (br s, 4H), 3.20 (br s, 4H). MS (M+H)⁺=377.0.

### Synthesis of 4-[(4,6-dichloro-3-quinolyl)sulfonyl]-1,4-thiazinane 1,1-dioxide (3)

6-chloro-3-[(1,1-dioxo-1,4-thiazinan-4-yl)sulfonyl]quinolin-4-ol (180 mg, 477.66 umol, 1 eq) was added to POCl₃ (9 mL) in portions and the mixture was stirred at 120° C. for 4 h under N₂ atmosphere. LCMS showed the starting material was consumed completely and desired MS was detected. The reaction mixture was concentrated to dryness to give the crude product. Then Ethyl acetate (10 mL) was added to it and poured into ice water (10 mL). The reaction mixture was filtered and the filter cake was concentrated in vacuo. Compound 4-[(4,6-dichloro-3-quinolyl)sulfonyl]-1,4-thiazinane 1,1-dioxide (90 mg, 227.69 umol, 47.67% yield) was obtained as a white solid. ¹H NMR (400 MHz, DMSO-d6) δ=8.95 (s, 1H), 8.21 (br s, 1H), 8.12 (br d, J=8.8 Hz, 1H), 7.88 (br d, J=8.0 Hz, 1H), 3.85 (br s, 2H), 3.32 (br s, 6H). MS (M+H)⁺=395.0.

### Synthesis of 5-chloro-2-[[6-chloro-3-[(1,1-dioxo-1,4-thiazinan-4-yl)sulfonyl]-4-quinolyl]amino]benzoic acid (429A)

To a solution of 4-[(4,6-dichloro-3-quinolyl)sulfonyl]-1,4-thiazinane 1,1-dioxide (80 mg, 202.39 umol, 1 eq) in EtOH (1 mL) and CHCl₃ (0.2 mL) was added 2-amino-5-chloro-benzoic acid (69.45 mg, 404.78 umol, 2 eq). The mixture was stirred at 80° C. for 2 h. LCMS showed the starting material was consumed completely and desired MS was detected. The reaction mixture was concentrated to dryness to give the crude product. The residue was purified by prep-HPLC (Phenomenex C18 80*40 mm*3 um column; 20-50% acetonitrile in an a 10 mM ammonium bicarbonate solution in water, 8 min gradient). Compound 5-chloro-2-[[6-chloro-3-[(1,1-dioxo-1,4-thiazinan-4-yl)sulfonyl]-4-quinolyl]amino]benzoic acid (14.90 mg, 28.09 umol, 13.88% yield, 100% purity) was obtained as a yellow solid. ¹H NMR (400 MHz, METHANOL-d₄) δ=9.19 (s, 1H), 8.09 (d, J=9.0 Hz, 1H), 8.05 (d, J=2.5 Hz, 1H), 7.83 (dd, J=2.3, 9.1 Hz, 1H), 7.72 (d, J=2.1 Hz, 1H), 7.25 (dd, J=2.5, 8.8 Hz, 1H), 6.56 (d, J=8.9 Hz, 1H), 3.82-3.64 (m, 4H), 3.20-3.00 (m, 4H). MS (M+H)⁺=530.0.

### Example 166—Synthesis of 430A

Synthetic scheme is provided in FIG. 39Y.

### Synthesis of 8-(4-bromo-6-chloro-3-quinolyl)-1,4-dioxa-8-azaspiro[4.5]decane (2)

A mixture of 4-bromo-6-chloro-3-iodo-quinoline (3 g, 8.14 mmol, 1 eq), 1,4-dioxa-8-azaspiro[4.5]decane (1.17 g, 8.14 mmol, 1.04 mL, 1 eq), t-BuONa (2.35 g, 24.43 mmol, 3 eq), BINAP (507.07 mg, 814.34 umol, 0.1 eq) and rac-BINAP-Pd-G3 (808.16 mg, 814.34 umol, 0.1 eq) in toluene (40 mL) was degassed and purged with N₂ for 3 times, then the mixture was stirred at 100° C. for 2 h under N₂ atmosphere. LCMS showed the starting material was consumed completely and desired MS was detected. 50 mL of water was added to the mixture and extracted with Ethyl acetate (50 mL*3). The combined organic layers were washed with brine (40 mL), dried over Na₂SO₄ and concentrated to dryness to give residue. The crude product was purified by flash column (ISCO 80 g silica, 0-55% ethyl acetate in petroleum ether, gradient over 15 min). Compound 8-(4-bromo-6-chloro-3-quinolyl)-1,4-dioxa-8-azaspiro[4.5]decane (1.67 g, 4.35 mmol, 53.45% yield) was obtained as a pale yellow solid. ¹H NMR (400 MHz, DMSO-d6) δ 8.82 (s, 1H), 8.08 (d, J=2.4 Hz, 1H), 8.02 (d, J=8.9 Hz, 1H), 7.71 (dd, J=2.3, 8.9 Hz, 1H), 3.95 (s, 4H), 3.31-3.27 (m, 4H), 1.86-1.82 (m, 4H). MS (M+H)⁺=3835.0.

### Synthesis of 5-chloro-2-[[6-chloro-3-(1,4-dioxa-8-azaspiro[4.5]decan-8-yl)-4-quinolyl]amino]benzoic acid (3)

A mixture of 8-(4-bromo-6-chloro-3-quinolyl)-1,4-dioxa-8-azaspiro[4.5]decane (1.6 g, 4.17 mmol, 1 eq), methyl 2-amino-5-chloro-benzoate (774.04 mg, 4.17 mmol, 1 eq), Cs₂CO₃ (2.72 g, 8.34 mmol, 2 eq) and rac-BINAP-Pd-G3 (413.86 mg, 417.03 umol, 0.1 eq) in tert-amyl alcohol (20 mL) was degassed and purged with N₂ for 3 times, and then the mixture was stirred at 90° C. for 12 hr under N₂ atmosphere. LCMS showed the starting material was consumed completely and desired MS was detected. The mixture was concentrated and acidified by HCl (2M) to pH=5-6 at 0° C., then extracted with Ethyl acetate (20 mL*3). The combined organic layers were washed with brine (15 mL) dried over Na₂SO₄ and concentrated to dryness to give residue. The crude product was purified by flash column (ISCO 40 g silica, 0-42% ethyl acetate in petroleum ether, gradient over 45 min). Compound 5-chloro-2-[[6-chloro-3-(1,4-dioxa-8-azaspiro[4.5]decan-8-yl)-4-quinolyl]amino]benzoic acid (940 mg, 1.98 mmol, 47.52% yield) was obtained as a yellow solid. ¹H NMR (400 MHz, DMSO-d6) δ 9.84 (br s, 1H), 8.82 (s, 1H), 7.99 (d, J=9.0 Hz, 1H), 7.88 (d, J=2.6 Hz, 1H), 7.77 (d, J=2.3 Hz, 1H), 7.62 (dd, J=2.1, 8.9 Hz, 1H), 7.35 (dd, J=2.6, 9.0 Hz, 1H), 6.46 (d, J=9.0 Hz, 1H), 3.85 (s, 4H), 3.10 (br s, 4H), 1.50 (br s, 4H). MS (M+H)⁺=474.10.

### Synthesis of 5-chloro-2-[[6-chloro-3-(4-oxo-1-piperidyl)-4-quinolyl]amino]benzoic acid (4)

To a solution of 5-chloro-2-[[6-chloro-3-(1,4-dioxa-8-azaspiro[4.5]decan-8-yl)-4-quinolyl]amino]benzoic acid (400 mg, 843.28 umol, 1 eq) in acetone (1 mL) was added HCl (3 M, 2 mL, 7.12 eq). The mixture was stirred at 70° C. for 1 h. LCMS showed the starting material was consumed completely and desired MS was detected. The reaction mixture was concentrated to dryness to give the crude product. Then the reaction mixture was cooled to 0° C. and sat·NaHCO₃ was added to it until PH=6-8. Then the reaction mixture was extracted with Ethyl acetate (10 mL*3). The combined organic layers were washed with brine (4 mL) and dried over Na₂SO₄. The combined organic layer was concentrated to dryness to give residue. Compound 5-chloro-2-[[6-chloro-3-(4-oxo-1-piperidyl)-4-quinolyl]amino]benzoic acid (310 mg, crude) was obtained as a yellow solid. MS (M+H)⁺=430.0.

### Synthesis of 5-chloro-2-[[6-chloro-3-(4,4-dichloro-1-piperidyl)-4-quinolyl]amino]benzoic acid (430A)

To a solution of 5-chloro-2-[[6-chloro-3-(4-oxo-1-piperidyl)-4-quinolyl]amino]benzoic acid (200 mg, 464.81 mmol, 1 eq) in DCM (2 mL) was added hexachlorotungsten (552.97 mg, 1.39 mmol, 3 eq). The mixture was stirred at 40° C. for 3 h. LCMS showed the starting material was consumed completely and desired MS was detected. The reaction mixture was concentrated to dryness to give the crude product. The crude product was purified by prep-HPLC (Phenomenex Luna 80*30 mm*3 um column; 25-55% acetonitrile in a 0.05% hydrochloric acid solution in water, 8 min gradient). Compound 5-chloro-2-[[6-chloro-3-(4,4-dichloro-1-piperidyl)-4-quinolyl]amino]benzoic acid (7.30 mg, 14.79 mmol, 1.59% yield) was obtained as a yellow solid. ¹H NMR (400 MHz, DMSO-d6) δ=10.19 (br s, 1H), 8.86 (s, 1H), 8.28 (br s, 1H), 8.07 (d, J=9.0 Hz, 1H), 7.91-7.83 (m, 2H), 7.61-7.50 (m, 1H), 7.08-6.92 (m, 1H), 3.06 (br s, 4H), 2.07 (br d, J=3.6 Hz, 4H). MS (M+H)⁺=484.0.

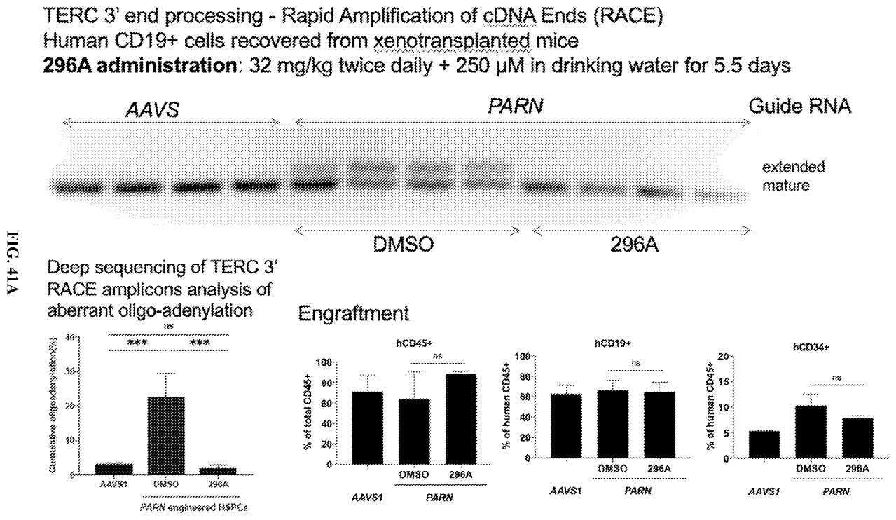

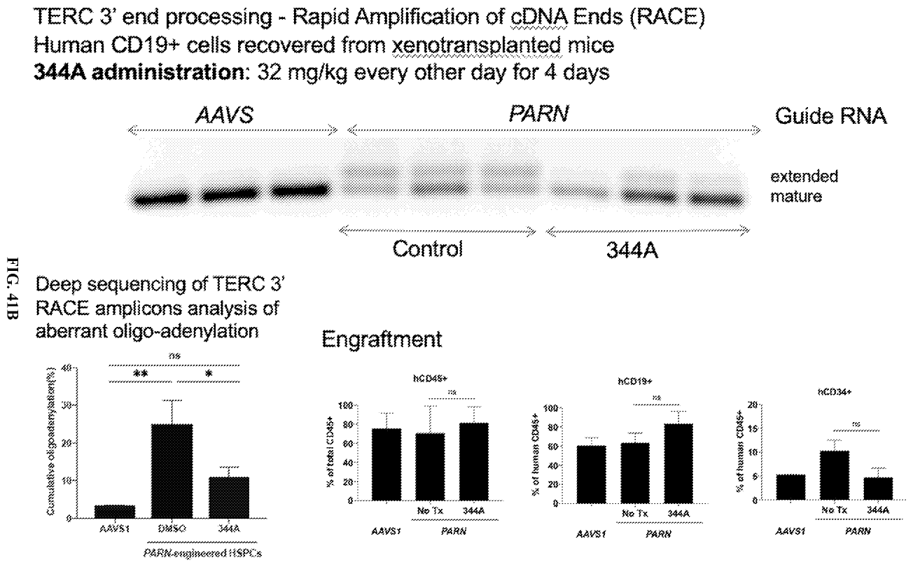

Example 167—Restoration of TERC 3′ end processing in primary CD34+ human hematopoietic stem and progenitor cells (HSPCs). To determine the efficacy of exemplified compounds in a disease-relevant stem cell population, we employed a highly-efficient CRISPR-Cas9 ribonucleoprotein transduction strategy to disrupt the PARN gene in primary human CD34+ hematopoietic stem and progenitor cells (HSPCs), or the control locus AAVS1. FIGS. 40A and 40B show extended TERC forms in CRISPR-Cas9 engineered HSPCs deficient in PARN. FIG. 40A shows maturation of TERC 3′ end processing—Rapid Amplification of cDNA Ends (RACE) for exemplified compounds 296A, 339A, 340A, 371A, 392A, 417A, 420A, 421A, 428A, and 396A tested at 1 μM in CRISPR/Cas9-engineered primary HSPCs on day 5. FIG. 40B shows maturation of TERC 3′ end processing—Rapid Amplification of cDNA Ends (RACE) for exemplified compounds 296A, 392A, 396A, 339A, 340A, 371A, 393A, and 404A tested at 100 nM in CRISPR/Cas9-engineered primary HSPCs on day 5.

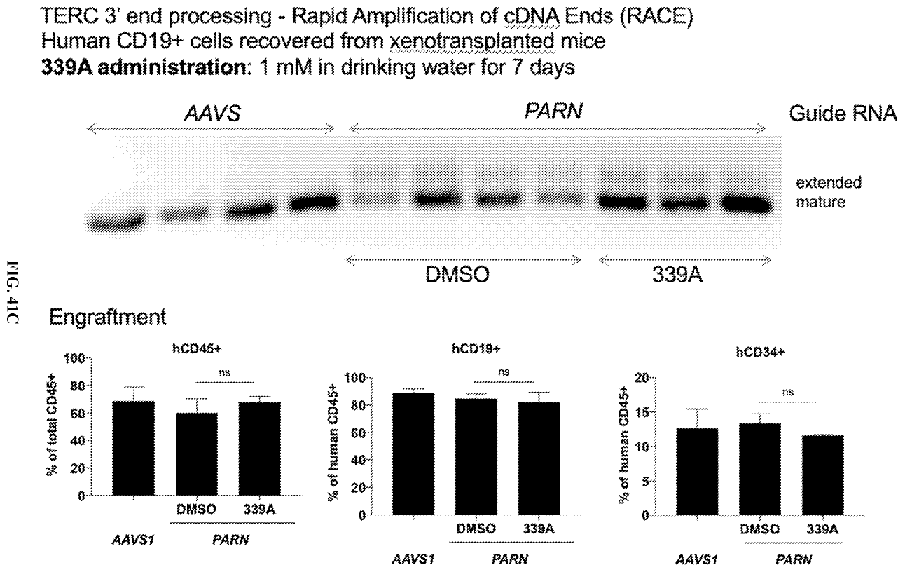

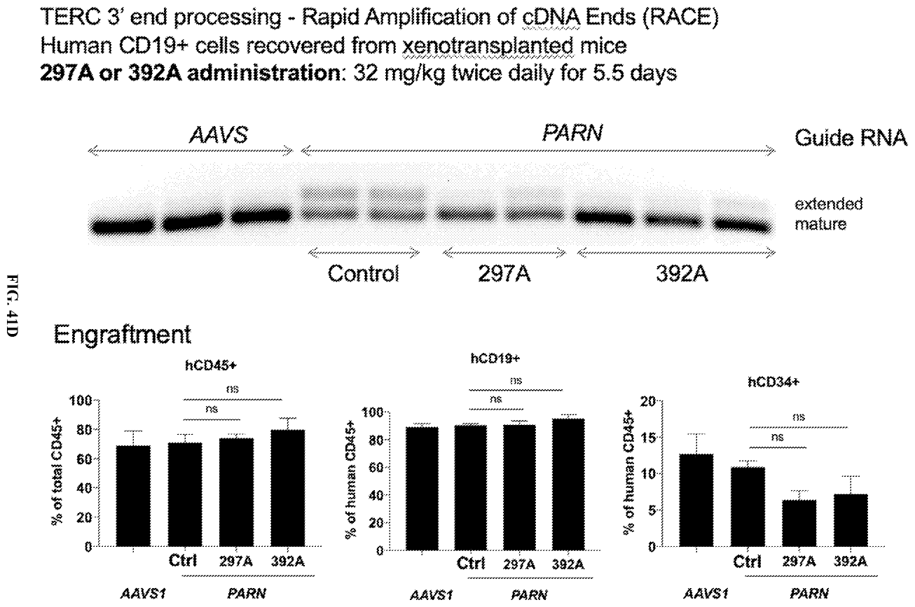

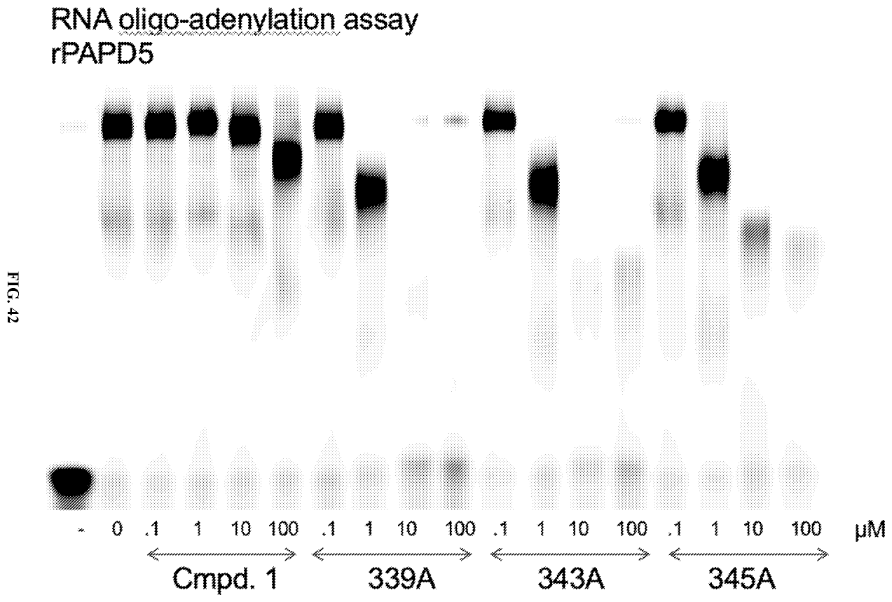

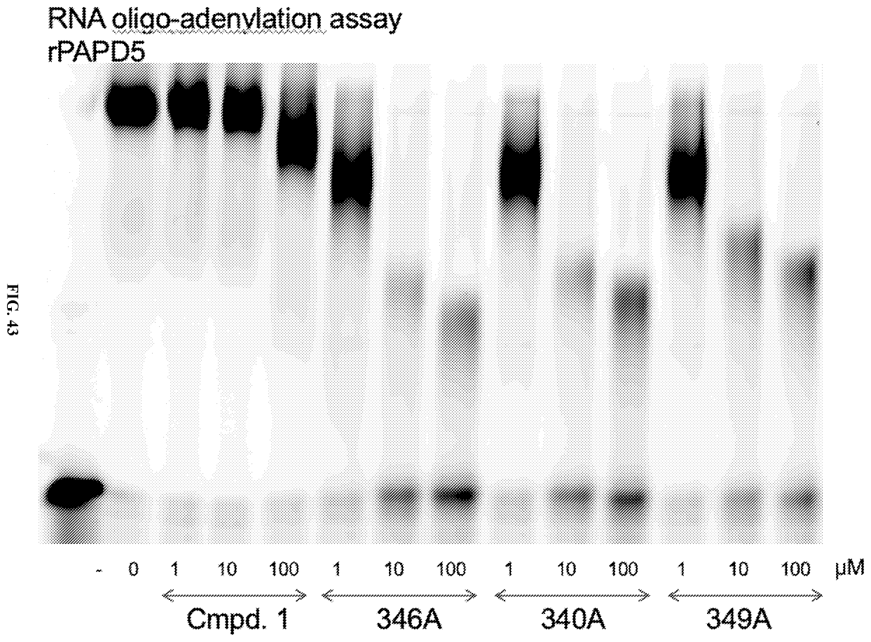

Example 168—Restoration of TERC 3′ end processing in vivo in human blood cells after xenotransplantation into immunodeficient mice. To determine the efficacy of orally administered exemplified compounds in primary human HSPCs and blood cells in restoring TERC processing in vivo, highly-efficient CRISPR-Cas9 ribonucleoprotein transduction was employed to disrupt the PARN gene in primary human CD34+ HSPCs or the control locus AAVS1. These genome-edited human HSPCs were xenotransplanted into immunodeficient NOD,B6.SCID Il2rg−/− KitW4l/W41 (NBSGW) mice, which engraft human HSPCs without exposure to radiation or chemotherapy. Six to ten weeks after xenotransplantation, exemplified compounds were administered for 4 to 7 days alongside controls. Thereafter, human hematopoietic cells were recovered from mouse bone marrow, and engraftment was analyzed by flow cytometry. Engrafted human hematopoietic cells were sorted for lineage markers, and CD19+ cells were used for analysis of restoration of TERC 3′ end processing by RNA-ligation mediated 3′ RACE. FIG. 41A shows maturation of TERC 3′ ends in human CD19+ cells recovered from mice xenotransplanted with HSPCs, for exemplified compound 296A administered at 32 mg/kg/dose twice daily for 11 doses, with simultaneous administration of 296A at 250 μM in drinking water. RACE amplicons were subjected to next-generation sequencing and oligo-adenylation was analyzed using a bioinformatics pipeline, showing that aberrant TERC oligo-adenylation in xenotransplanted PARN-deficient human blood cells was significantly reversed in vivo by exemplified compound 296A oral administration. Analysis of engraftment shows no change in engraftment of CD45+ human blood cells, CD19+ human blood cells, CD34+ human blood cells after exemplified compound 296A treatment. FIG. 41B shows maturation of TERC 3′ ends in human CD19+ cells recovered from mice xenotransplanted with HSPCs, for exemplified compound 344 administered at 32 mg/kg/dose every other day for 4 days. RACE amplicons were subjected to next-generation sequencing and oligo-adenylation was analyzed using bioinformatics pipelines, showing aberrant TERC oligo-adenylation in xenotransplanted PARN-deficient human blood cells was significantly reversed in vivo by exemplified compound 344A oral administration. Analysis of engraftment shows no change in engraftment of CD45+ human blood cells, CD19+ human blood cells, CD34+ human blood cells after exemplified compound 344A treatment. FIG. 41C shows maturation of TERC 3′ ends in human CD19+ cells recovered from mice xenotransplanted with HSPCs, for exemplified compound 339A administered at 1 mM in drinking water for 7 days. Analysis of engraftment shows no change in engraftment of CD45+ human blood cells, CD19+ human blood cells, CD34+ human blood cells after exemplified compound 339A treatment. FIG. 41D shows maturation of TERC 3′ ends in human CD19+ cells recovered from mice xenotransplanted with HSPCs, for exemplified compounds 297A or 392A administered at 32 mg/kg/dose twice daily for 11 doses. Analysis of engraftment shows no change in engraftment of CD45+ human blood cells, CD19+ human blood cells, CD34+ human blood cells after exemplified compound 297A or 392A treatment.

### REFERENCES

1. Neha Nagpal et al., Small-Molecule PAPD5 inhibitors restore telomerase activity in patient stem cells, Cell Stem Cell, 26 (2020), 1-14.
2. Wilson Chun Fok et al., Posttranslational modulation of TERC by PAPD5 inhibition rescues hematopoietic development in dyskeratosis congenita, Blood, 144, 12 (2019), 1308-1312.

### Numbered Paragraphs

In some embodiments, the invention disclosed herein can be described with reference to the following numbered paragraphs.

Paragraph 1. A compound of Formula (I):

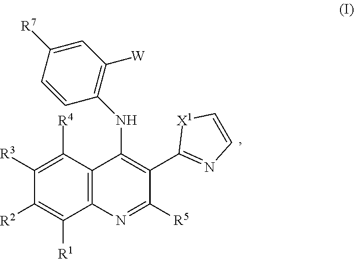

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, C₁₋₃ alkyl, and C₁₋₃ alkoxy.

Paragraph 2. The compound of paragraph 1, wherein the compound of Formula (I) is selected from any one of the compounds listed in Table I, or a pharmaceutically acceptable salt thereof.

Paragraph 3. A compound of Formula (II):

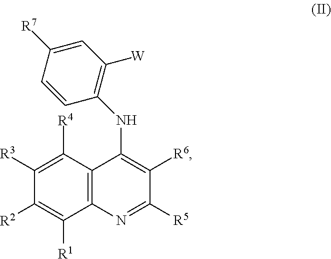

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo;
R⁶ is a 5-membered heteroaryl selected from the group consisting of:

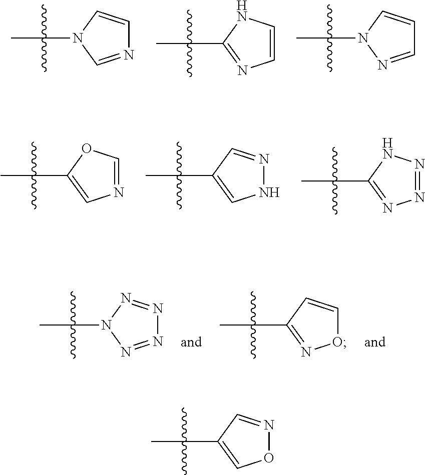

R⁷ is selected from halo, C₁₋₃ alkyl, and C₁₋₃ alkoxy.

Paragraph 4. The compound of paragraph 3, wherein the compound of Formula (II) is selected from any one of the compounds listed in Table II, or a pharmaceutically acceptable salt thereof.

Paragraph 5. A compound of Formula (III)

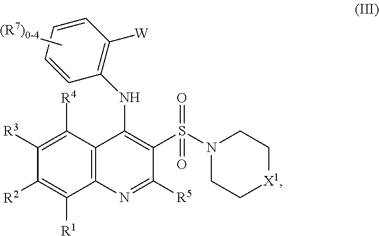

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
R³ is a 5-membered heteroaryl, optionally substituted with 1, 2, or 3 substituents independently selected from C₁₋₆ alkyl, C₁₋₄ haloalkyl, C₁₋₆ alkylcarbonyl, CN, halo, C₁₋₆ alkoxy, C₁₋₆ alkoxy-C₁₋₆ alkyl, NO₂, C₁₋₆ haloalkoxy, cyano-C₁₋₃ alkylene, C₃₋₁₀ cycloalkyl, 4-6 membered heterocycloalkyl, amino, C₁₋₆ alkylamino, di(C₁₋₆ alkyl)amino, carboxy, and C₁₋₆ alkoxycarbonyl;
W is selected from C(O)OR⁸ and a carboxylic acid bioisostere;
R⁸ is selected from H and C₁₋₆ alkyl; and
each R⁷ is selected from halo, C₁₋₃ alkyl, C₁₋₃ haloalkyl, C₁₋₃ haloalkoxy, and C₁₋₃ alkoxy.

Paragraph 6. The compound of paragraph 5, wherein the compound of Formula (III) is selected from any one of the compounds listed in Table III, or a pharmaceutically acceptable salt thereof.

Paragraph 7. A compound of Formula (IV)

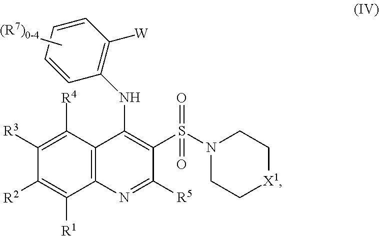

or a pharmaceutically acceptable salt thereof, wherein.
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
R³ is selected from pyridinyl and pyrimidinyl, each of which is optionally substituted with 1, 2, or 3 substituents independently selected from C₁₋₆ alkyl. C₁₋₄ haloalkyl, C₁₋₆ alkylcarbonyl, CN, OH, halo, C₁₋₆ alkoxy, C₁₋₆ alkoxy-C₁₋₆ alkyl, C₆₋₁₀ aryl, C₆₋₁₀ aryloxy, NO₂, C₁₋₆ haloalkoxy, cyano-C₁₋₃ alkylene, C₃₋₁₀ cycloalkyl, 4-6 membered heterocycloalkyl, amino, C₁₋₆ alkylamino, di(C₁₋₆ alkyl)amino, carboxy, C₁₋₆ alkylsulfonyl, C₁₋₆ alkoxy carbonyl, carbamyl, C₁₋₆ alkylcarbamyl, and di(C₁₋₆ alkyl)carbamyl;
W is selected from C(O)OR⁸ and a carboxylic acid bioisostere;
R⁸ is selected from H and C₁₋₆ alkyl; and
each R⁷ is independently selected from halo, C₁₋₃ alkyl, C₁₋₃ haloalkyl, C₁₋₃ haloalkoxy, and C₁₋₃ alkoxy.

Paragraph 8. The compound of paragraph 7, wherein the compound of Formula (IV) is selected from any one of the compounds listed in Table IV, or a pharmaceutically acceptable salt thereof.

Paragraph 9. A compound of Formula (V):

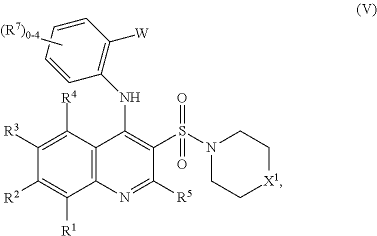

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ a 9 to 10-membered heteroaryl selected from the group consisting of:

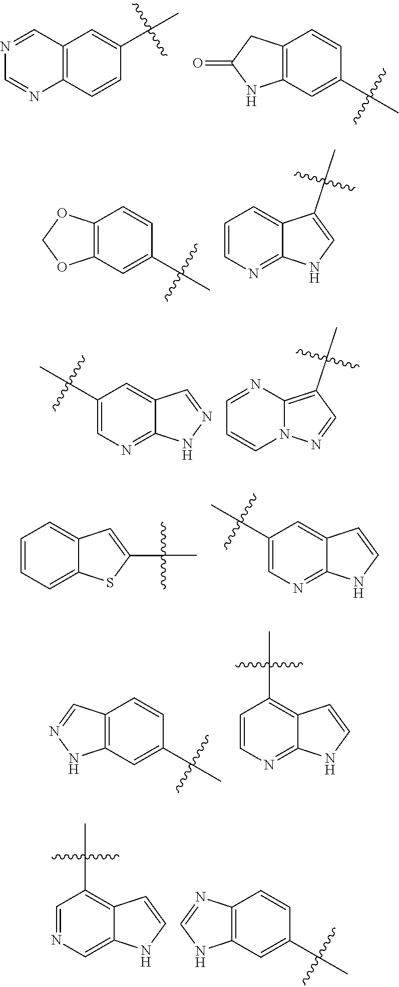

each of which is optionally substituted with 1, 2, or 3 substituents independently selected from C₁₋₆ alkyl, C₁₋₄ haloalkyl, C₁₋₆ alkylcarbonyl, CN, OH, halo, C₁₋₆ alkoxy, C₁₋₆ alkoxy-C₁₋₆ alkyl, C₆₋₁₀ aryl, C₆₋₁₀ aryloxy, NO₂, C₁₋₆ haloalkoxy, cyano-C₁₋₃ alkylene, C₃₋₁₀ cycloalkyl, 4-6 membered heterocycloalkyl, amino, C₁₋₆ alkylamino, di(C₁₋₆ alkyl)amino, carboxy, C₁₋₆ alkylsulfonyl, C₆₋₁₀ arylsulfonyl, C₁₋₆ alkoxycarbonyl, carbamyl, C₁₋₆ alkylcarbamyl, and di(C₁₋₆ alkyl)carbamyl; and
each R⁷ is selected from halo, C₁₋₃ alkyl, C₁₋₃ haloalkyl, C₁₋₃ haloalkoxy, and C₁₋₃ alkoxy.

Paragraph 10. The compound of paragraph 9, wherein the compound of Formula (V) is selected from any one of the compounds listed in Table V, or a pharmaceutically acceptable salt thereof.

Paragraph 11. A compound of Formula (VI):

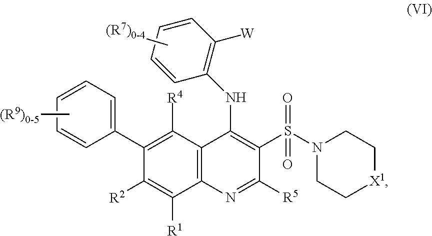

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
each R⁹ is independently selected from C₁₋₆ alkyl, C₁₋₄ haloalkyl, C₁₋₆ alkylcarbonyl, CN, OH, halo, C₁₋₆ alkoxy, C₁₋₆ alkoxy-C₁₋₆ alkyl, C₆₋₁₀ aryl, C₆₋₁₀ aryloxy, NO₂, C₁₋₆ haloalkoxy, cyano-C₁₋₃ alkylene, C₃₋₁₀ cycloalkyl, 4-6 membered heterocycloalkyl, 5-6 membered heteroaryl, amino, C₁₋₆ alkylamino, di(C₁₋₆ alkyl)amino, carboxy, C₁₋₆ alkylsulfonyl, C₆₋₁₀ arylsulfonyl, 5-6 membered heterocycloalkylsulfonyl, C₁₋₆ alkoxycarbonyl, carbamyl, C₁₋₆ alkylcarbamyl, di(C₁₋₆ alkyl)carbamyl, C₁₋₆ alkylsulfonylamino, wherein said 6 membered heterocycloalkyl and 5-6 membered heteroaryl are each optionally substituted with 1 or 2 substituents independently selected from C₁₋₆ alkyl, C₁₋₄ haloalkyl, C₁₋₆ alkoxy, and C₁₋₄ haloalkoxy; and
each R⁷ is selected from halo, C₁₋₃ alkyl, C₁₋₃ haloalkyl, C₁₋₃ haloalkoxy, and C₁₋₃ alkoxy.

Paragraph 12. The compound of paragraph 11, wherein the compound of Formula (VI) is selected from any one of the compounds listed in Table VI, or a pharmaceutically acceptable salt thereof.

Paragraph 13. A compound of Formula (VII):

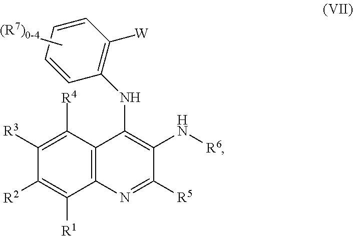

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is selected from 5-6 membered heterocycloalkyl, C₄₋₆ cycloalkyl, and a 5-6 membered heteroaryl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl;
R³ is halo; and
each R⁷ is independently selected from halo, C₁₋₃ alkyl, C₁₋₃ haloalkyl, C₁₋₃ haloalkoxy, and C₁₋₃ alkoxy.

Paragraph 14. The compound of paragraph 13, wherein the compound of Formula (VII) is selected from any one of the compounds listed in Table VII, or a pharmaceutically acceptable salt thereof.

Paragraph 15. A compound of Formula (VIII):

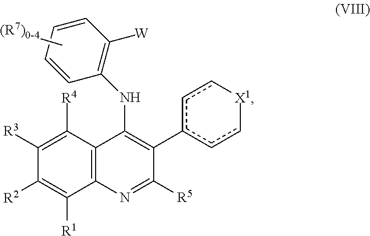

or a pharmaceutically acceptable salt thereof, wherein:
X¹ is selected from O, S, CF₂, C═O, CHCl, CHF, CCl₂, C═N—OH, NH, NCH₃, Si(OH)₂, SO₂, and cyclopropylidene;
each [symbol] is independently a single bond or a double bond, provided that no more than two of [symbol] are double bonds;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

Paragraph 16. The compound of paragraph 15, wherein X¹ is selected from O, S, CF₂, CHCl, CCl₂, NH, NCH₃, Si(OH)₂, SO₂, and cyclopropylidene.

Paragraph 17. The compound of paragraph 15, wherein the compound of Formula (VIII) is selected from any one of the compounds listed in Table VIII, or a pharmaceutically acceptable salt thereof.

Paragraph 18. A compound of Formula (IX):

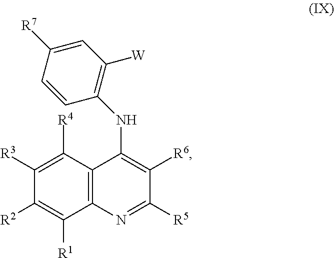

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is a 5-membered heterocycloalkyl;
R³ is halo; and
R⁷ is selected from halo, C₁₋₃ alkyl, and C₁₋₃ alkoxy.

Paragraph 19. The compound of paragraph 18, wherein the compound of Formula (IX) is selected from any one of the compounds listed in Table IX, or a pharmaceutically acceptable salt thereof.

Paragraph 20. A compound of Formula (X):

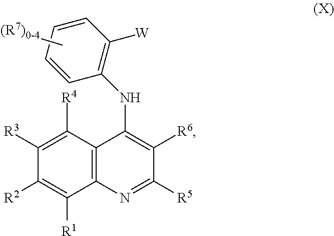

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R⁶ is a 6-membered heteroaryl;
R³ is halo; and
R⁷ is selected from halo, C₁₋₃ alkyl, and C₁₋₃ alkoxy.

Paragraph 21. The compound of paragraph 20, wherein the compound of Formula (X) is selected from any one of the compounds listed in Table X, or a pharmaceutically acceptable salt thereof.

Paragraph 22. A compound of Formula (XI):

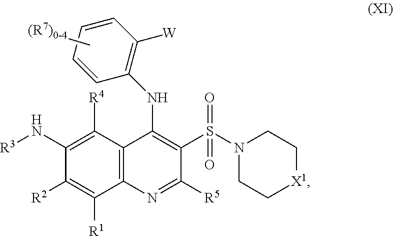

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is selected from 5-6 membered heterocycloalkyl, C₄₋₆ cycloalkyl, and 5-9 membered heteroaryl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl; and
R⁷ is selected from halo, C₁₋₃ alkyl, and C₁₋₃ alkoxy.

Paragraph 23. The compound of paragraph 22, wherein the compound of Formula (XI) is selected from any one of the compounds listed in Table XI, or a pharmaceutically acceptable salt thereof.

Paragraph 24. A compound of Formula (XII):

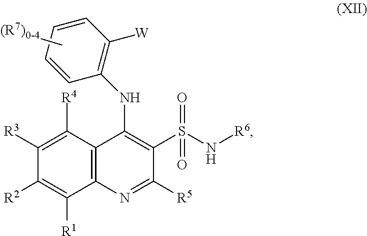

or a pharmaceutically acceptable salt thereof, wherein:
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R^{R} is selected from H and C₁₋₆ alkyl;
R⁶ is selected from 5-6 membered heterocycloalkyl, C₄₋₆ cycloalkyl, C₆₋₁₀ aryl, and a 5-6 membered heteroaryl, each of which is optionally substituted with 1 or 2 substituents independently selected from NO₂, CN, halo, C₁₋₃ alkyl, C₁₋₄ haloalkyl, C₁₋₃ alkoxy, C₁₋₃ haloalkoxy, amino, C₁₋₃ alkylamino, di(C₁₋₃ alkyl)amino, carboxy, C₁₋₆ alkylcarbonyl, and C₁₋₆ alkoxycarbonyl;
R³ is halo; and
each R⁷ is independently selected from halo, C₁₋₃ alkyl, C₁₋₃ haloalkyl, C₁₋₃ haloalkoxy, and C₁₋₃ alkoxy.

Paragraph 25. The compound of paragraph 24, wherein the compound of Formula (XII) is selected from any one of the compounds listed in Table XII, or a pharmaceutically acceptable salt thereof.

Paragraph 26. A compound of Formula (XIII):

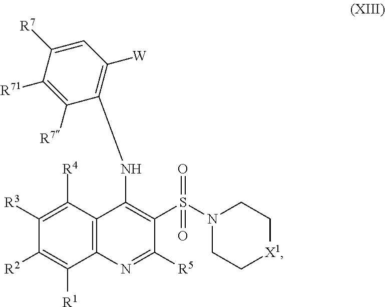

or a pharmaceutically acceptable salt thereof, wherein:
X¹ is selected from O, S, and SO₂;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo;
R⁷ is selected from C═O(OH), halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy; and
R^{7′} and R^{7″} are each independently selected from H, halo, CN, C₁₋₃ alkyl, and C₁₋₃ haloalkyl.

Paragraph 27. The compound of paragraph 26, having formula:

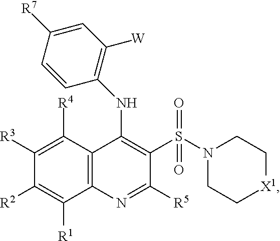

or a pharmaceutically acceptable salt thereof, wherein:
X¹ is selected from O and S; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

Paragraph 28. The compound of paragraph 26, wherein the compound of Formula (XIII) is selected from any one of the compounds listed in Table XIII, or a pharmaceutically acceptable salt thereof.

Paragraph 29. A compound of Formula (XIV):

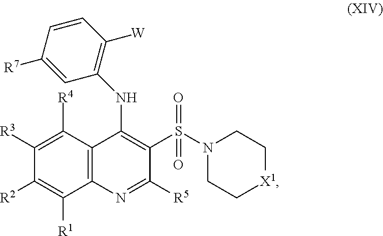

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

Paragraph 30. The compound of paragraph 29, wherein the compound of Formula (XIV) is selected from any one of the compounds listed in Table XIV, or a pharmaceutically acceptable salt thereof.

Paragraph 31. A compound of Formula (XV):

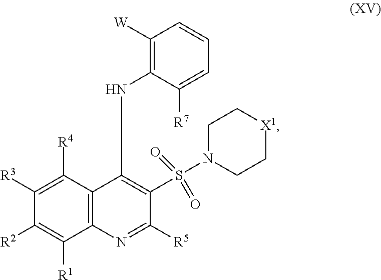

or a pharmaceutically acceptable salt thereof, wherein:
X¹ is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

Paragraph 32. The compound of paragraph 31, wherein the compound of Formula (XV) is selected from any one of the compounds listed in Table XV, or a pharmaceutically acceptable salt thereof.

Paragraph 33. A compound of Formula (XVI):

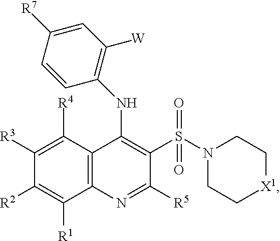

or a pharmaceutically acceptable salt thereof, wherein:
X¹ is selected from O, S, CF₂, C═0, C═N—OH, CHOH, CHCl, CHF, CH(OCF₃), CCl₂, NH, NCH₃, Si(OH)₂, SO₂, and cyclopropylidene;
each, [symbol] is independently a single bond or a double bond;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

Paragraph 34. The compound of paragraph 33, wherein X¹ is selected from O, S, CF₂, C═N—OH, CHCl, CC12, NH, NCH₃, Si(OH)₂, SO₂, and cyclopropylidene.

Paragraph 35. The compound of paragraph 33, wherein the compound of Formula (XVI) is selected from any one of the compounds listed in Table XVI, or a pharmaceutically acceptable salt thereof.

Paragraph 36. A compound of Formula (XVII):

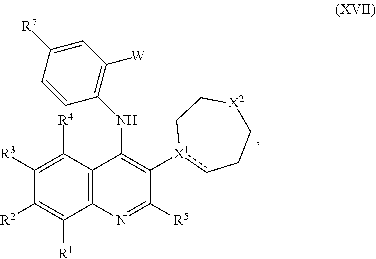

or a pharmaceutically acceptable salt thereof, wherein:
X¹ is N or CH when [symbol] is a single bond;
X¹ is C when [symbol] is a double bond;
X² is selected from O and S;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

Paragraph 37. The compound of paragraph 36, wherein the compound of Formula (XVII) is selected from any one of the compounds listed in Table XVII, or a pharmaceutically acceptable salt thereof.

Paragraph 38. A compound of Formula (XVIII):

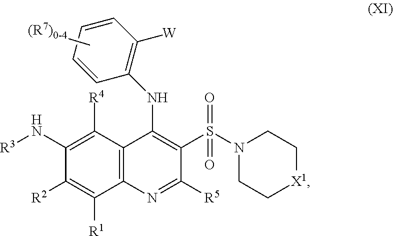

or a pharmaceutically acceptable salt thereof, wherein:
X¹ is selected from O and S;
X² is selected from CH₂, CHCH₃, and C(CH₃)₂;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
R⁷ is selected from halo, B(OH)₂, OH, CN, C₁₋₃ alkyl, C₁₋₃ haloalkyl, HO—C₁₋₃ haloalkyl, aminosulfonyl, C₁₋₃ haloalkylcarbonyl, C₁₋₃ alkylcarbonyl, carbamyl, and C₁₋₃ alkoxy.

Paragraph 39. The compound of paragraph 38, wherein the compound of Formula (XVIII) is selected from any one of the compounds listed in Table I, or a pharmaceutically acceptable salt thereof.

Paragraph 40. A compound selected from any one of the compounds listed in Table 1A and Tables 2A-2E, or a pharmaceutically acceptable salt thereof.

Paragraph 41. A pharmaceutical composition comprising a compound of any one of paragraphs paragraph 1-40, or a pharmaceutically acceptable salt thereof, and a pharmaceutically acceptable carrier.

Paragraph 42. A method of treating or preventing a disease or condition selected from: a disorder associated with telomere or telomerase dysfunction, a disorder associated with aging, a pre-leukemic or pre-cancerous condition, an HBV infection, an HAV infection, a CMV infection, a neurodevelopmental disorder, and an acquired or genetic disease or condition associated with alterations in RNA, the method comprising administering to a subject in need thereof a therapeutically effective amount of a compound of any one of paragraph 1-40, or a pharmaceutically acceptable salt thereof.

Paragraph 43. The method of paragraph 42, wherein the disorder associated with telomere or telomerase dysfunction is dyskeratosis congenita, aplastic anemia, myelodysplastic syndrome, pulmonary fibrosis, interstitial lung disease, hematological disorder, liver disease or hepatic fibrosis.

Paragraph 44. The method of paragraph 42, wherein the disorder associated with aging is macular degeneration, diabetes mellitus, osteoarthritis, rheumatoid arthritis, sarcopenia, cardiovascular disease, hypertension, atherosclerosis, coronary artery disease, ischemia/reperfusion injury, cancer, premature death, or age-related decline in cognitive function, cardiopulmonary function, muscle strength, vision, or hearing.

Paragraph 45. The method of paragraph 42, wherein the neurodevelopmental disorder is pontocerebellar hypoplasia.

Paragraph 46. A method of expanding a cell, the method comprising culturing the cell in the presence of an effective amount of a compound of any one of paragraphs 1-40, or a pharmaceutically acceptable salt thereof.

Paragraph 47. The method of paragraph 46, wherein the cell is selected from the group consisting of stem cell, pluripotent stem cell, hematopoietic stem cell, and embryonic stem cell.

Paragraph 48. The method of paragraph 46, wherein the cell is collected from a subject with a disease or condition selected from the group consisting of a disorder associated with telomere or telomerase dysfunction, a disorder associated with aging, a pre-leukemic or pre-cancerous condition, and a neurodevelopment disorder.

Paragraph 49. The method of paragraph 46, wherein the cell is a Chimeric Antigen Receptor (CAR) T-Cell.

Paragraph 50. The method of paragraph 46, wherein the cell is a T cell, an engineered T cell, or a natural killer cell (NK).

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (VIII): 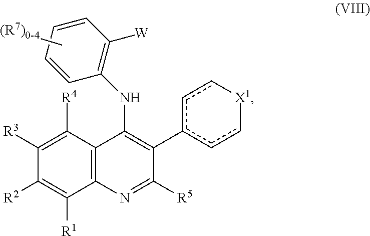
or a pharmaceutically acceptable salt thereof, wherein:
X¹ is selected from O, S, CF₂, C═O, CHCl, CHF, CCl₂, C═N—OH, NH, NCH₃, Si(OH)₂, SO₂, and cyclopropylidene;
each [symbol] is independently a single bond or a double bond, provided that no more than two of [symbol] are double bonds;
R¹, R², R⁴, and R⁵ are each independently selected from H, C₁₋₃ alkyl, C₁₋₃ alkoxy, C₁₋₄ haloalkyl, C₁₋₄ haloalkoxy, halo, CN, and NO₂;
W is selected from C(O)OR⁸ and a carboxylic acid bioisostere;
R⁸ is selected from H and C₁₋₆ alkyl;
R³ is halo; and
each R⁷ is independently selected from halo, C₁₋₃ alkyl, C₁₋₃ haloalkyl, C₁₋₃ haloalkoxy, and C₁₋₃ alkoxy.

2. The compound of claim 1, wherein X¹ is selected from O, S, CF₂, CHCl, CCl₂, NH, NCH₃, Si(OH)₂, SO₂, and cyclopropylidene.

3. The compound of claim 1, wherein R¹, R², R⁴, and R⁵ are each independently selected from H, C₁₋₃ alkyl, C₁₋₃ alkoxy, C₁₋₄ haloalkyl, C₁₋₄ haloalkoxy, and halo.

4. The compound of claim 1, wherein R¹, R², R⁴, and R⁵ are each independently selected from H and C₁₋₃ alkyl.

5. The compound of claim 1, wherein C(O)OR⁸.

6. The compound of claim 1, wherein W is a carboxylic acid bioisostere.

7. The compound of claim 6, wherein W is selected from any one of the following moieties:

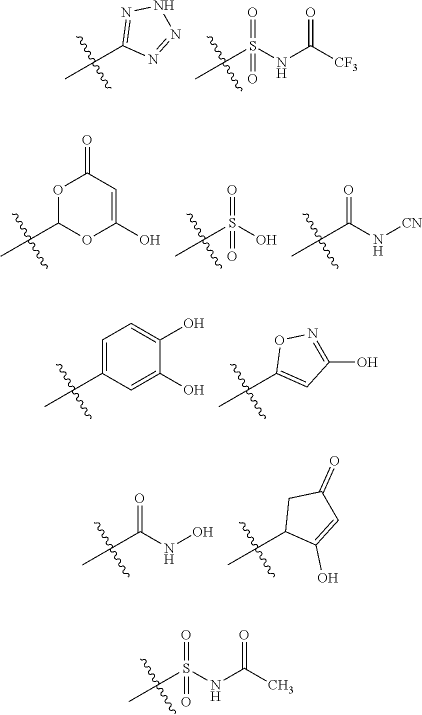

8. The compound of claim 1, wherein the compound of Formula (VIII) has formula: 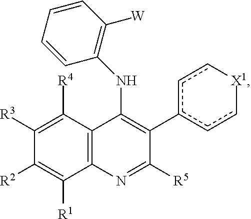
or a pharmaceutically acceptable salt thereof.

9. The compound of claim 1, wherein the compound of Formula (VIII) has formula: 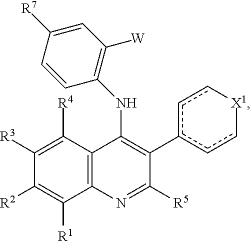
or a pharmaceutically acceptable salt thereof.

10. The compound of claim 1, having formula: 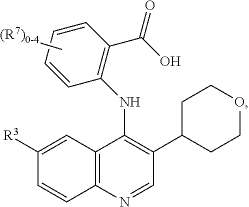
or a pharmaceutically acceptable salt thereof, wherein:
R³ is selected from Cl, Br, and F; and
R⁷ is selected from halo, C₁₋₃ alkyl, and C₁₋₃ alkoxy.

11. The compound of claim 1, having any one of the following formulae: 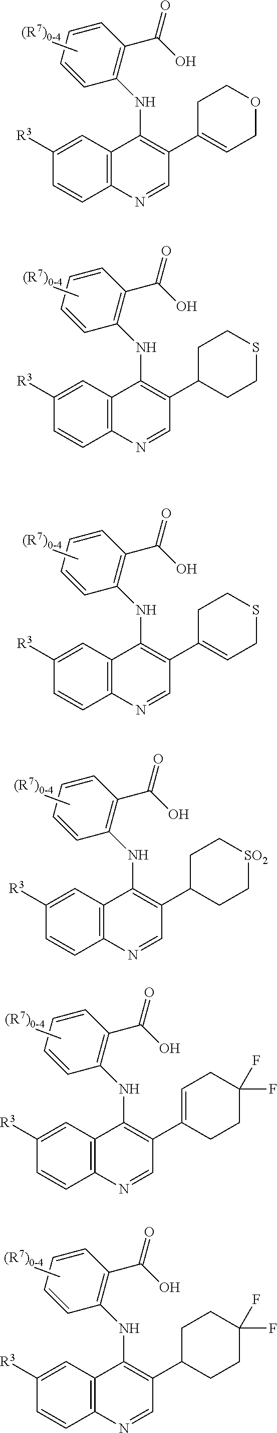 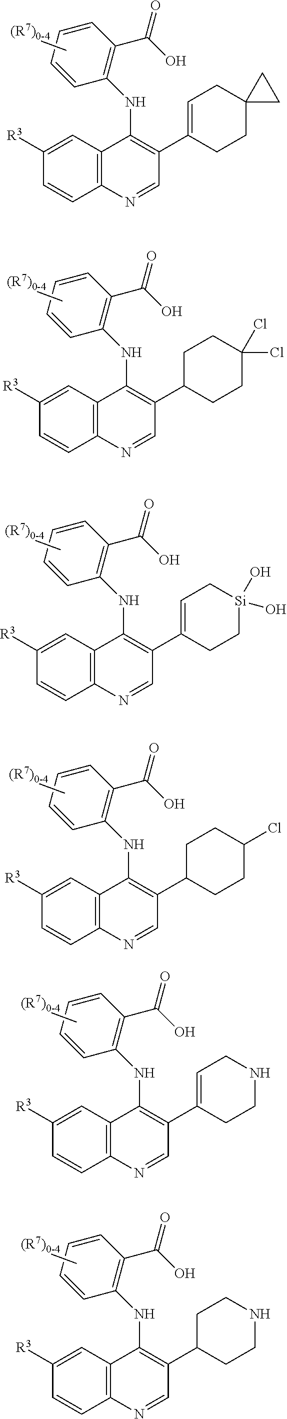 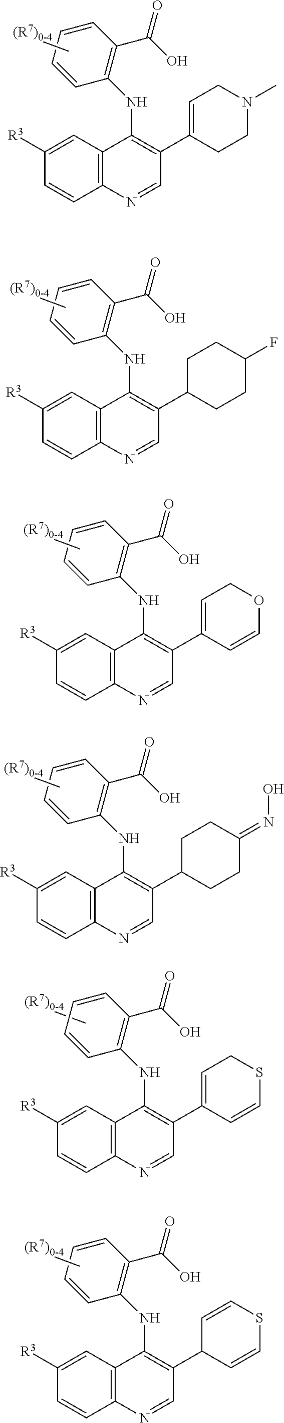
or a pharmaceutically acceptable salt thereof, wherein:
R³ is selected from Cl, Br, and F; and
R⁷ is selected from halo, C₁₋₃ alkyl, and C₁₋₃ alkoxy.

12. The compound of claim 1, wherein the compound is selected from any one of the following compounds: 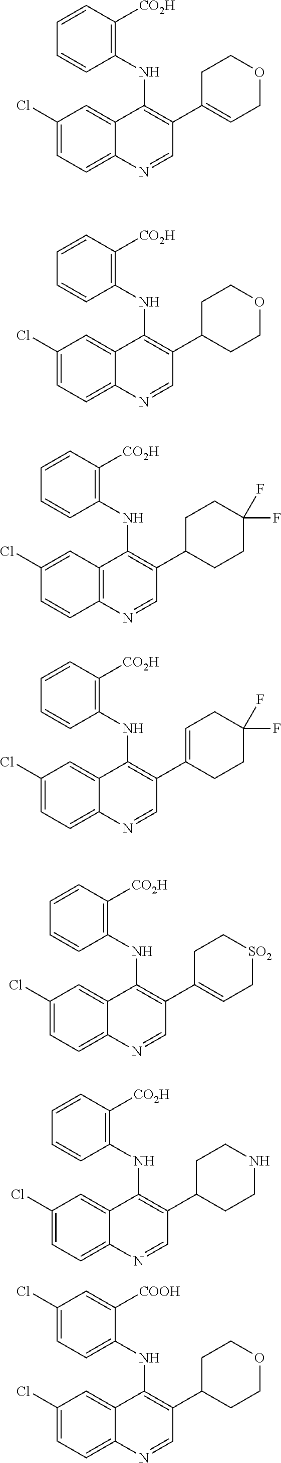 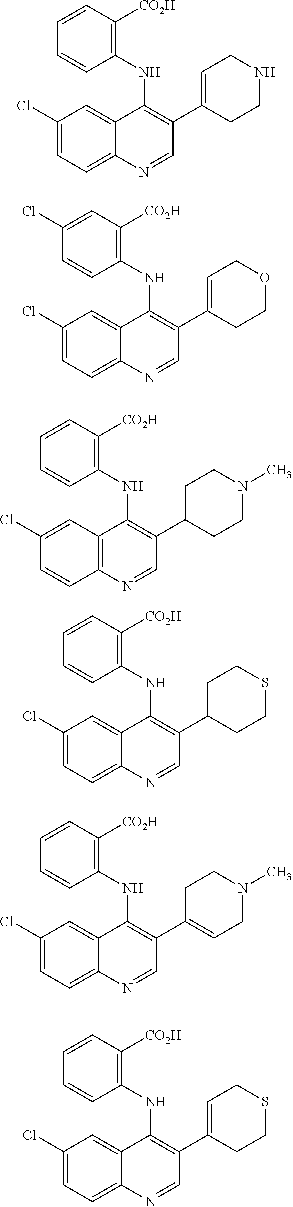 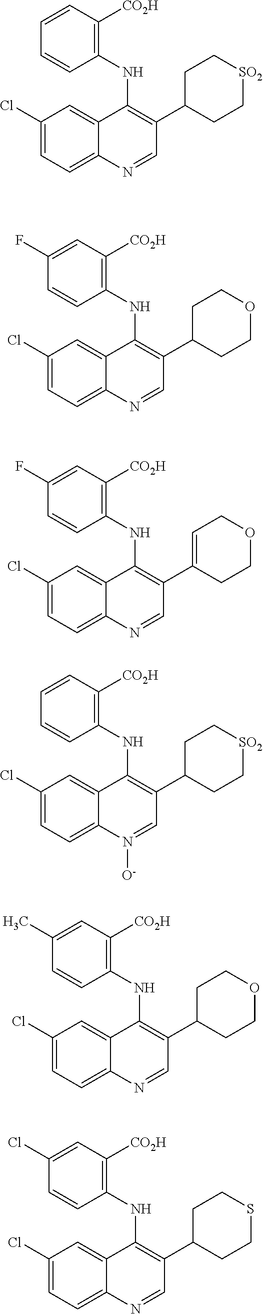 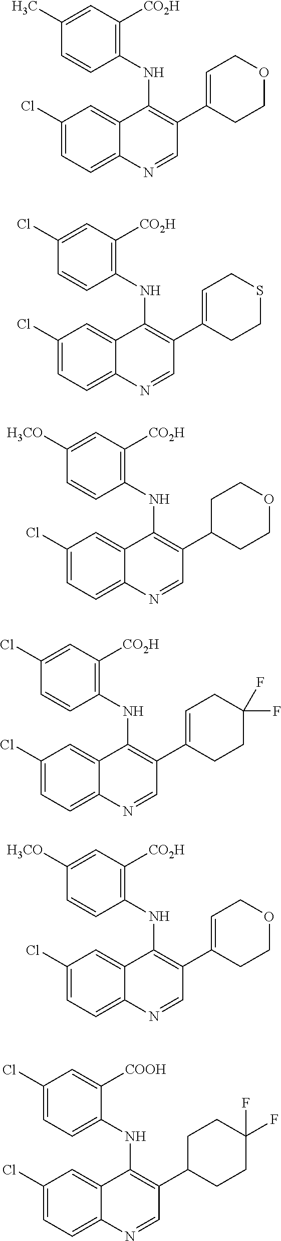 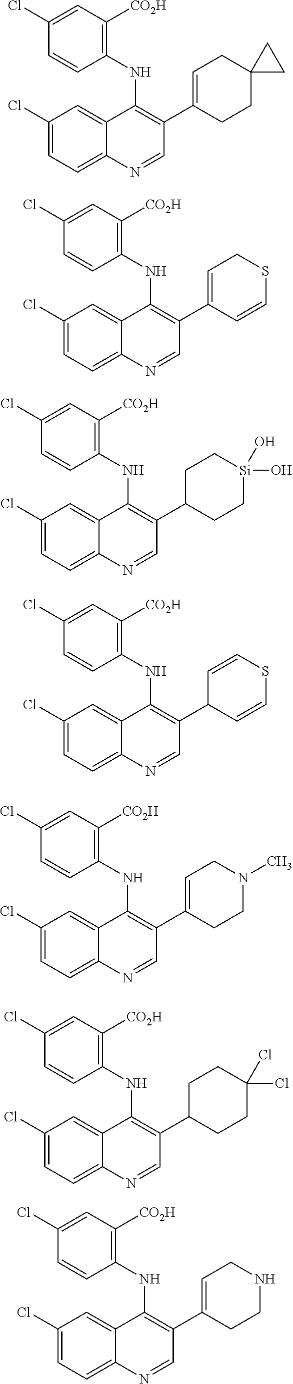 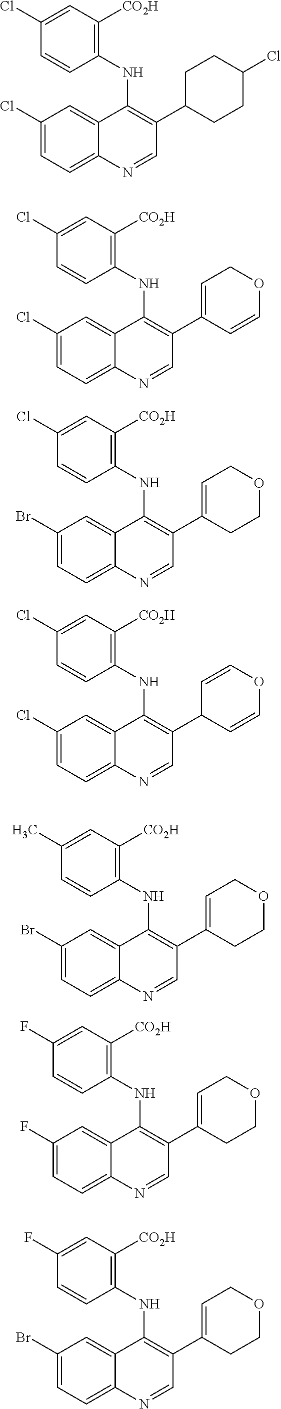 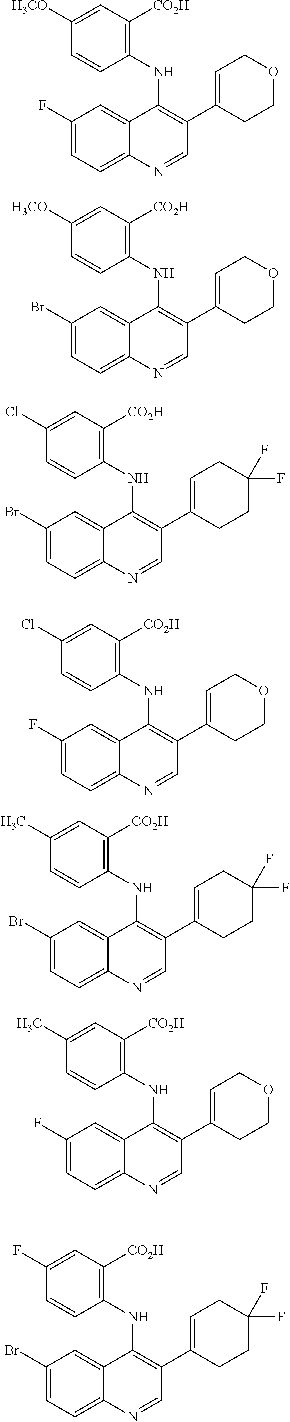 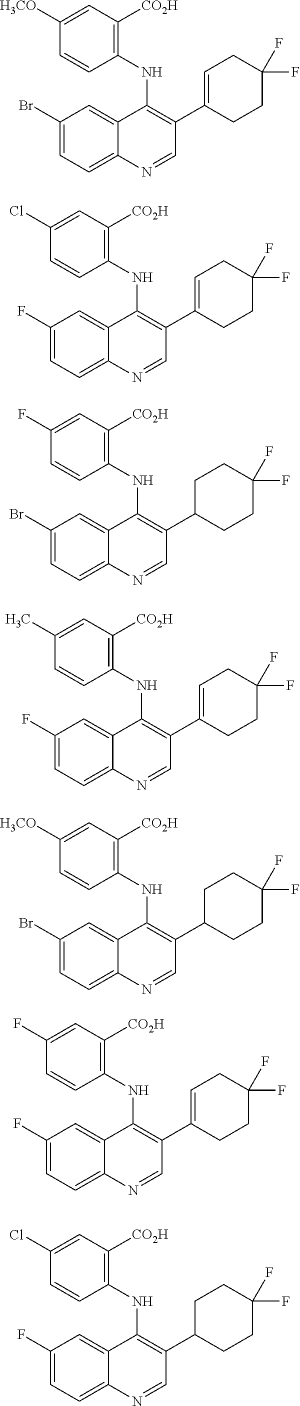 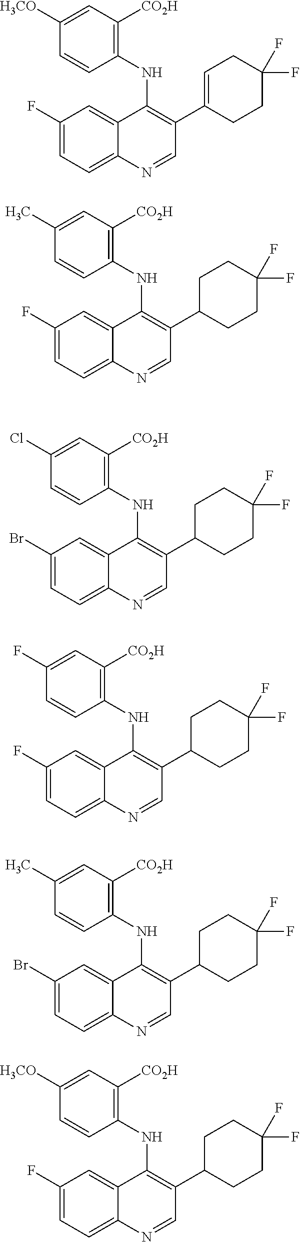 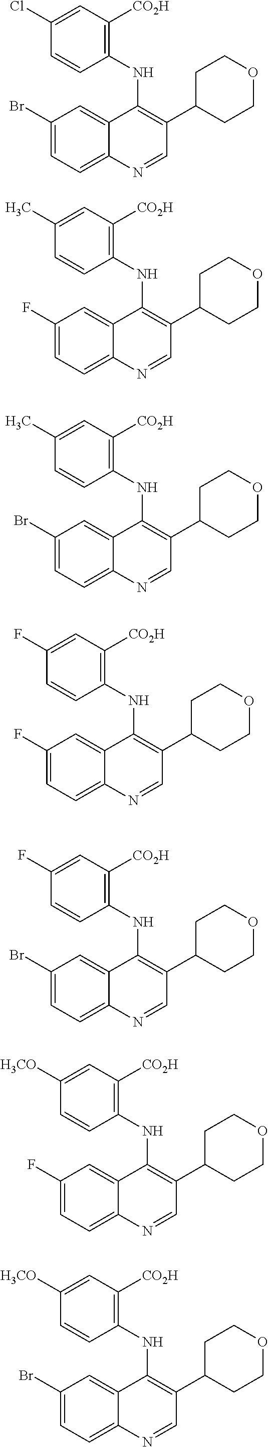 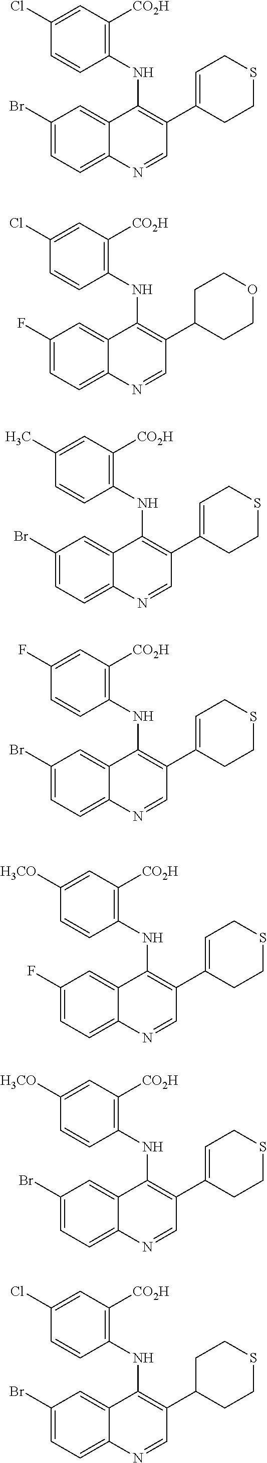 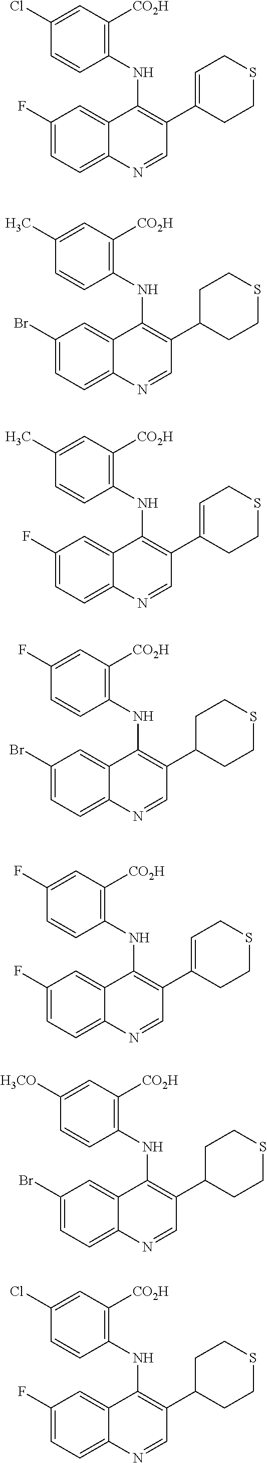 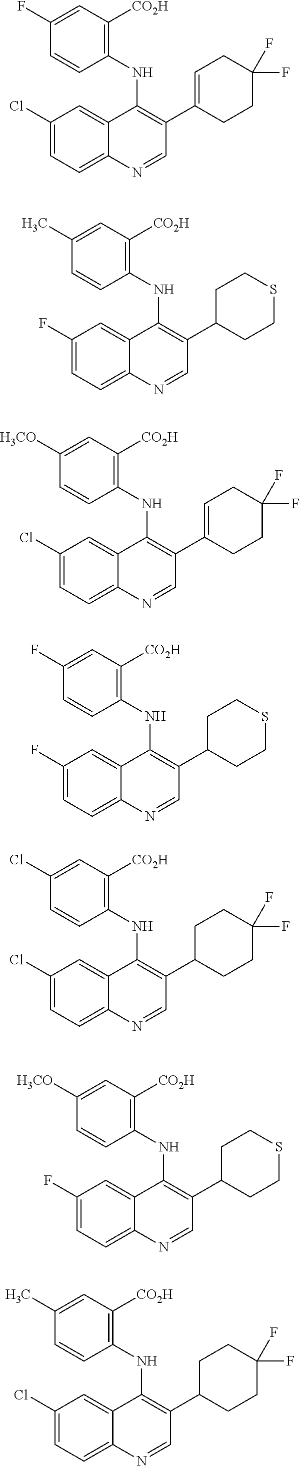 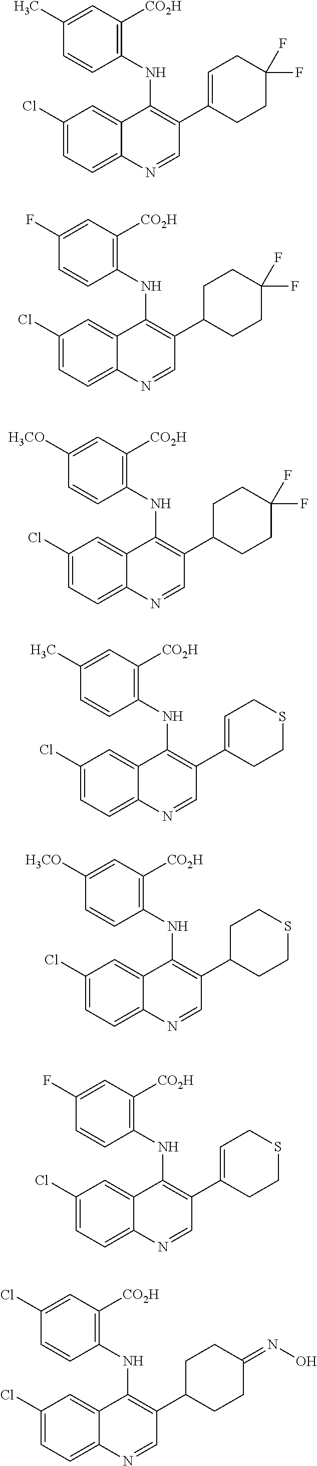 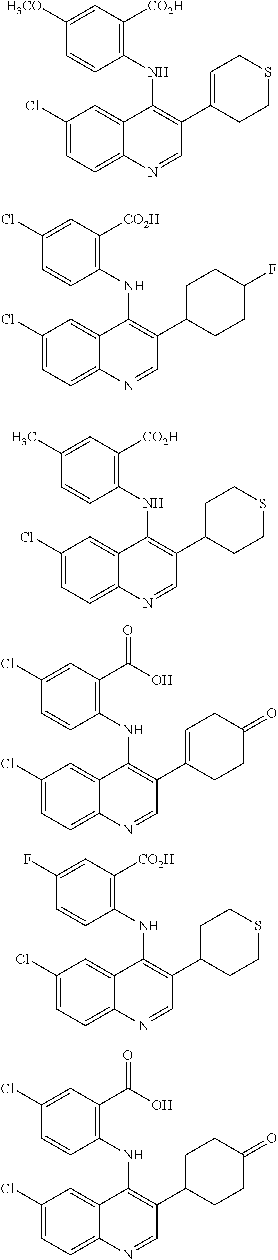
or a pharmaceutically acceptable salt thereof.

13. A pharmaceutical composition comprising a compound of claim 1, or a pharmaceutically acceptable salt thereof, and a pharmaceutically acceptable carrier.

14. A method of treating or preventing a disease or condition selected from: a disorder associated with telomere or telomerase dysfunction, a disorder associated with aging, a pre-leukemic or pre-cancerous condition, an HBV infection, an HAV infection, a CMV infection, a neurodevelopmental disorder, and an acquired or genetic disease or condition associated with alterations in RNA, the method comprising administering to a subject in need thereof a therapeutically effective amount of a compound of claim 1, or a pharmaceutically acceptable salt thereof.

15. The method of claim 14, wherein the disorder associated with telomere or telomerase dysfunction is dyskeratosis congenita, aplastic anemia, myelodysplastic syndrome, pulmonary fibrosis, interstitial lung disease, hematological disorder, liver disease or hepatic fibrosis.

16. The method of claim 14, wherein the disorder associated with aging is macular degeneration, diabetes mellitus, osteoarthritis, rheumatoid arthritis, sarcopenia, cardiovascular disease, hypertension, atherosclerosis, coronary artery disease, ischemia/reperfusion injury, cancer, premature death, or age-related decline in cognitive function, cardiopulmonary function, muscle strength, vision, or hearing.

17. The method of claim 14, wherein the neurodevelopmental disorder is pontocerebellar hypoplasia.

18. A method of expanding a cell, the method comprising culturing the cell in the presence of an effective amount of a compound of claim 1, or a pharmaceutically acceptable salt thereof.

19. The method of claim 18, wherein the cell is selected from the group consisting of: stem cell, pluripotent stem cell, hematopoietic stem cell, and embryonic stem cell.

20. The method of claim 18, wherein the cell is collected from a subject with a disease or condition selected from the group consisting of a disorder associated with telomere or telomerase dysfunction, a disorder associated with aging, a pre-leukemic or pre-cancerous condition, and a neurodevelopment disorder.

21-22. (canceled)
